SEQUENCE LISTING (1480 sequences)

SEQ ID NO: 1 - DNA, 24 bp, Artificial Sequence
  Description of Artificial Sequence Synthetic primer
aaactagataacagtagacatcac

SEQ ID NO: 2 - DNA, 29 bp, Artificial Sequence
  Description of Artificial Sequence Synthetic primer
aaccttaattggaaagaaacccaagaagt

SEQ ID NO: 3 - DNA, 17 bp, Artificial Sequence
  Description of Artificial Sequence Synthetic primer
aacgcacaatcagaagc

SEQ ID NO: 4 - DNA, 25 bp, Artificial Sequence
  Description of Artificial Sequence Synthetic primer
aactaccgtccgcagttctacttcc

SEQ ID NO: 5 - DNA, 25 bp, Artificial Sequence
  Description of Artificial Sequence Synthetic primer
aactaccgtcctcagttctacttcc

SEQ ID NO: 6 - DNA, 18 bp, Artificial Sequence
  Description of Artificial Sequence Synthetic primer
aagacgacctgcacgggc

SEQ ID NO: 7 - DNA, 18 bp, Artificial Sequence
  Description of Artificial Sequence Synthetic primer
aagcggtggagcatgtgg

SEQ ID NO: 8 - DNA, 26 bp, Artificial Sequence
  Description of Artificial Sequence Synthetic primer
aaggaaggcgtgatcaccgttgaaga

SEQ ID NO: 9 - DNA, 23 bp, Artificial Sequence
  Description of Artificial Sequence Synthetic primer
aaggtactccggggataacaggc

SEQ ID NO: 10 - DNA, 22 bp, Artificial Sequence
  Description of Artificial Sequence Synthetic primer
aagtcggaatcgctagtaatcg

SEQ ID NO: 11 - DNA, 19 bp, Artificial Sequence
  Description of Artificial Sequence Synthetic primer
aatctgctatttggtcagg

SEQ ID NO: 12 - DNA, 22 bp, Artificial Sequence
  Description of Artificial Sequence Synthetic primer
acaacgaagtacaatacaagac

SEQ ID NO: 13 - DNA, 21 bp, Artificial Sequence
  Description of Artificial Sequence Synthetic primer
acaatacaagacaaaagaagg

SEQ ID NO: 14 - DNA, 19 bp, Artificial Sequence
  Description of Artificial Sequence Synthetic primer
accacgccgtaaacgatga

SEQ ID NO: 15 - DNA, 24 bp, Artificial Sequence
  Description of Artificial Sequence Synthetic primer
accatgacagaaggcattttgaca

SEQ ID NO: 16 - DNA, 22 bp, Artificial Sequence
  Description of Artificial Sequence Synthetic primer
acccagtgctgctgaaccgtgc

SEQ ID NO: 17 - DNA, 19 bp, Artificial Sequence
  Description of Artificial Sequence Synthetic primer
accgagcaaggagaccagc

SEQ ID NO: 18 - DNA, 19 bp, Artificial Sequence
  Description of Artificial Sequence Synthetic primer
acctgcccagtgctggaag

SEQ ID NO: 19 - DNA, 17 bp, Artificial Sequence
  Description of Artificial Sequence Synthetic primer
acgcgaagaaccttacc

SEQ ID NO: 20 - DNA, 20 bp, Artificial Sequence
  Description of Artificial Sequence Synthetic primer
actcgtttttaatcagcccg

SEQ ID NO: 21 - DNA, 20 bp, Artificial Sequence
  Description of Artificial Sequence Synthetic primer
agaacaccgatggcgaaggc

SEQ ID NO: 22 - DNA, 23 bp, Artificial Sequence
  Description of Artificial Sequence Synthetic primer
agaatcaagttcccaggggttac

SEQ ID NO: 23 - DNA, 20 bp, Artificial Sequence
  Description of Artificial Sequence Synthetic primer
agagtttgatcatggctcag

SEQ ID NO: 24 - DNA, 28 bp, Artificial Sequence
  Description of Artificial Sequence Synthetic primer
agcaggtggtgaaatcggccacatgatt

SEQ ID NO: 25 - DNA, 18 bp, Artificial Sequence
  Description of Artificial Sequence Synthetic primer
agcgtaaaggtgaacctt

SEQ ID NO: 26 - DNA, 26 bp, Artificial Sequence
  Description of Artificial Sequence Synthetic primer
agcttttgcatattatatcgagccac

SEQ ID NO: 27 - DNA, 28 bp, Artificial Sequence
  Description of Artificial Sequence Synthetic primer
aggacagagtgagtactttgaccgaggt

SEQ ID NO: 28 - DNA, 22 bp, Artificial Sequence
  Description of Artificial Sequence Synthetic primer
agtctcaagagtgaacacgtaa

SEQ ID NO: 29 - DNA, 32 bp, Artificial Sequence
  Description of Artificial Sequence Synthetic primer
agttataaacacggctttcctatggcttatcc

SEQ ID NO: 30 - DNA, 21 bp, Artificial Sequence
  Description of Artificial Sequence Synthetic primer
atactcctgactgaccgatag

SEQ ID NO: 31 - DNA, 20 bp, Artificial Sequence
  Description of Artificial Sequence Synthetic primer
atatcgacggcggtgtttgg

SEQ ID NO: 32 - DNA, 25 bp, Artificial Sequence
  Description of Artificial Sequence Synthetic primer
atcaatttggtggccaagaacctgg

SEQ ID NO: 33 - DNA, 29 bp, Artificial Sequence
  Description of Artificial Sequence Synthetic primer
atgattacaattcaagaaggtcgtcacgc

SEQ ID NO: 34 - DNA, 23 bp, Artificial Sequence
  Description of Artificial Sequence Synthetic primer
atggacaaggttggcaaggaagg

SEQ ID NO: 35 - DNA, 20 bp, Artificial Sequence
  Description of Artificial Sequence Synthetic primer
atggccatggcagaagctca

SEQ ID NO: 36 - DNA, 25 bp, Artificial Sequence
  Description of Artificial Sequence Synthetic primer
atgtcgattgcaatccgtacttgtg

SEQ ID NO: 37 - DNA, 19 bp, Artificial Sequence
  Description of Artificial Sequence Synthetic primer
atgttgggttaagtcccgc

SEQ ID NO: 38 - DNA, 25 bp, Artificial Sequence
  Description of Artificial Sequence Synthetic primer
atgttgggttaagtcccgcaacgag

SEQ ID NO: 39 - DNA, 28 bp, Artificial Sequence
  Description of Artificial Sequence Synthetic primer
caaaacttattaggtaagcgtgttgact

SEQ ID NO: 40 - DNA, 27 bp, Artificial Sequence
  Description of Artificial Sequence Synthetic primer
caaaggtaagcaaggacgtttccgtca

SEQ ID NO: 41 - DNA, 27 bp, Artificial Sequence
  Description of Artificial Sequence Synthetic primer
caaaggtaagcaaggtcgtttccgtca

SEQ ID NO: 42 - DNA, 17 bp, Artificial Sequence
  Description of Artificial Sequence Synthetic primer
caacgagcgcaaccctt

SEQ ID NO: 43 - DNA, 17 bp, Artificial Sequence
  Description of Artificial Sequence Synthetic primer
caacggatgctggcaag

SEQ ID NO: 44 - DNA, 31 bp, Artificial Sequence
  Description of Artificial Sequence Synthetic primer
caagaagaaaaagagcttctaaaaagaatac

SEQ ID NO: 45 - DNA, 23 bp, Artificial Sequence
  Description of Artificial Sequence Synthetic primer
caagcaaacgcacaatcagaagc

SEQ ID NO: 46 - DNA, 19 bp, Artificial Sequence
  Description of Artificial Sequence Synthetic primer
caagtcatcatggccctta

SEQ ID NO: 47 - DNA, 26 bp, Artificial Sequence
  Description of Artificial Sequence Synthetic primer
caataccgcaacagcggtggcttggg

SEQ ID NO: 48 - DNA, 19 bp, Artificial Sequence
  Description of Artificial Sequence Synthetic primer
cactggaactgagacacgg

SEQ ID NO: 49 - DNA, 20 bp, Artificial Sequence
  Description of Artificial Sequence Synthetic primer
cagaatcaagttcccagggg

SEQ ID NO: 50 - DNA, 24 bp, Artificial Sequence
  Description of Artificial Sequence Synthetic primer
cagagaccgttttatcctatcagc

SEQ ID NO: 51 - DNA, 20 bp, Artificial Sequence
  Description of Artificial Sequence Synthetic primer
cagcgtttcggcgaaatgga

SEQ ID NO: 52 - DNA, 29 bp, Artificial Sequence
  Description of Artificial Sequence Synthetic primer
caggagtcgttcaactcgatctacatgat

SEQ ID NO: 53 - DNA, 24 bp, Artificial Sequence
  Description of Artificial Sequence Synthetic primer
caggtttagtaccagaacatgcag

SEQ ID NO: 54 - DNA, 23 bp, Artificial Sequence
  Description of Artificial Sequence Synthetic primer
catccacacggtggtggtgaagg

SEQ ID NO: 55 - DNA, 23 bp, Artificial Sequence
  Description of Artificial Sequence Synthetic primer
ccacacgccgttcttcaacaact

SEQ ID NO: 56 - DNA, 28 bp, Artificial Sequence
  Description of Artificial Sequence Synthetic primer
ccacagttctacttccgtactactgacg

SEQ ID NO: 57 - DNA, 19 bp, Artificial Sequence
  Description of Artificial Sequence Synthetic primer
ccagcagccgcggtaatac

SEQ ID NO: 58 - DNA, 19 bp, Artificial Sequence
  Description of Artificial Sequence Synthetic primer
ccgtaacttcgggagaagg

SEQ ID NO: 59 - DNA, 20 bp, Artificial Sequence
  Description of Artificial Sequence Synthetic primer
ccgtggtattggagttattg

SEQ ID NO: 60 - DNA, 29 bp, Artificial Sequence
  Description of Artificial Sequence Synthetic primer
cctatattaatcgtttacagaaactggct

SEQ ID NO: 61 - DNA, 19 bp, Artificial Sequence
  Description of Artificial Sequence Synthetic primer
cctgataagggtgaggtcg

SEQ ID NO: 62 - DNA, 30 bp, Artificial Sequence
  Description of Artificial Sequence Synthetic primer
ccttacttcgaactatgaatcttttggaag

SEQ ID NO: 63 - DNA, 14 bp, Artificial Sequence
  Description of Artificial Sequence Synthetic primer
cgaagaaccttacc

SEQ ID NO: 64 - DNA, 27 bp, Artificial Sequence
  Description of Artificial Sequence Synthetic primer
cgaagtacaatacaagacaaaagaagg

SEQ ID NO: 65 - DNA, 18 bp, Artificial Sequence
  Description of Artificial Sequence Synthetic primer
cgacgcgctgcgcttcac

SEQ ID NO: 66 - DNA, 22 bp, Artificial Sequence
  Description of Artificial Sequence Synthetic primer
cgagagggaaacaacccagacc

SEQ ID NO: 67 - DNA, 30 bp, Artificial Sequence
  Description of Artificial Sequence Synthetic primer
cgagtatagctaaaaaaatagtttatgaca

SEQ ID NO: 68 - DNA, 23 bp, Artificial Sequence
  Description of Artificial Sequence Synthetic primer
cgcaaaaaaatccagctattagc

SEQ ID NO: 69 - DNA, 20 bp, Artificial Sequence
  Description of Artificial Sequence Synthetic primer
cgccgacttcgacggtgacc

SEQ ID NO: 70 - DNA, 20 bp, Artificial Sequence
  Description of Artificial Sequence Synthetic primer
cggaattactgggcgtaaag

SEQ ID NO: 71 - DNA, 21 bp, Artificial Sequence
  Description of Artificial Sequence Synthetic primer
cggattggagtctgcaactcg

SEQ ID NO: 72 - DNA, 22 bp, Artificial Sequence
  Description of Artificial Sequence Synthetic primer
cggcgtacttcaacgacagcca

SEQ ID NO: 73 - DNA, 24 bp, Artificial Sequence
  Description of Artificial Sequence Synthetic primer
cgtaactataacggtcctaaggta

SEQ ID NO: 74 - DNA, 26 bp, Artificial Sequence
  Description of Artificial Sequence Synthetic primer
cgtcagggtaaattccgtgaagttaa

SEQ ID NO: 75 - DNA, 26 bp, Artificial Sequence
  Description of Artificial Sequence Synthetic primer
cgtcgggtgattaaccgtaacaaccg

SEQ ID NO: 76 - DNA, 26 bp, Artificial Sequence
  Description of Artificial Sequence Synthetic primer
cgtcgtgtaattaaccgtaacaaccg

SEQ ID NO: 77 - DNA, 20 bp, Artificial Sequence
  Description of Artificial Sequence Synthetic primer
cgtggcggcgtggttatcga

SEQ ID NO: 78 - DNA, 25 bp, Artificial Sequence
  Description of Artificial Sequence Synthetic primer
cgtgttgactattcggggcgttcag

SEQ ID NO: 79 - DNA, 18 bp, Artificial Sequence
  Description of Artificial Sequence Synthetic primer
ctagtacgagaggaccgg

SEQ ID NO: 80 - DNA, 18 bp, Artificial Sequence
  Description of Artificial Sequence Synthetic primer
ctgacacctgcccggtgc

SEQ ID NO: 81 - DNA, 24 bp, Artificial Sequence
  Description of Artificial Sequence Synthetic primer
ctggcaggtatgcgtggtctgatg

SEQ ID NO: 82 - DNA, 23 bp, Artificial Sequence
  Description of Artificial Sequence Synthetic primer
ctggctaaaactttggcaacggt

SEQ ID NO: 83 - DNA, 24 bp, Artificial Sequence
  Description of Artificial Sequence Synthetic primer
ctgtccctagtacgagaggaccgg

SEQ ID NO: 84 - DNA, 22 bp, Artificial Sequence
  Description of Artificial Sequence Synthetic primer
ctgttcttagtacgagaggacc

SEQ ID NO: 85 - DNA, 24 bp, Artificial Sequence
  Description of Artificial Sequence Synthetic primer
cttctgcaacaagctgtggaacgc

SEQ ID NO: 86 - DNA, 24 bp, Artificial Sequence
  Description of Artificial Sequence Synthetic primer
cttgctggtatgcgtggtctgatg

SEQ ID NO: 87 - DNA, 29 bp, Artificial Sequence
  Description of Artificial Sequence Synthetic primer
cttggaggtaagtctcattttggtgggca

SEQ ID NO: 88 - DNA, 19 bp, Artificial Sequence
  Description of Artificial Sequence Synthetic primer
cttgtacacaccgcccgtc

SEQ ID NO: 89 - DNA, 30 bp, Artificial Sequence
  Description of Artificial Sequence Synthetic primer
cttgtacttgtggctcacacggctgtttgg

SEQ ID NO: 90 - DNA, 21 bp, Artificial Sequence
  Description of Artificial Sequence Synthetic primer
cttttgcatattatatcgagc

SEQ ID NO: 91 - DNA, 20 bp, Artificial Sequence
  Description of Artificial Sequence Synthetic primer
gaatagcaattaatccaaat

SEQ ID NO: 92 - DNA, 18 bp, Artificial Sequence
  Description of Artificial Sequence Synthetic primer
gaaagagttcggattggg

SEQ ID NO: 93 - DNA, 21 bp, Artificial Sequence
  Description of Artificial Sequence Synthetic primer
gaaggatatacggttgatgtc

SEQ ID NO: 94 - DNA, 20 bp, Artificial Sequence
  Description of Artificial Sequence Synthetic primer
gaatagcaattaatccaaat

SEQ ID NO: 95 - DNA, 20 bp, Artificial Sequence
  Description of Artificial Sequence Synthetic primer
gacacggtccagactcctac

SEQ ID NO: 96 - DNA, 18 bp, Artificial Sequence
  Description of Artificial Sequence Synthetic primer
gacagttcggtccctatc

SEQ ID NO: 97 - DNA, 18 bp, Artificial Sequence
  Description of Artificial Sequence Synthetic primer
gaccacctcggcaaccgt

SEQ ID NO: 98 - DNA, 30 bp, Artificial Sequence
  Description of Artificial Sequence Synthetic primer
gacctacagtaagaggttctgtaatgaacc

SEQ ID NO: 99 - DNA, 16 bp, Artificial Sequence
  Description of Artificial Sequence Synthetic primer
gacgcctgcccggtgc

SEQ ID NO: 100 - DNA, 20 bp, Artificial Sequence
  Description of Artificial Sequence Synthetic primer
gacttaccaacccgatgcaa

SEQ ID NO: 101 - DNA, 20 bp, Artificial Sequence
  Description of Artificial Sequence Synthetic primer
gagagcaagcggacctcata

SEQ ID NO: 102 - DNA, 27 bp, Artificial Sequence
  Description of Artificial Sequence Synthetic primer
gagagtttgatcctggctcagaacgaa

SEQ ID NO: 103 - DNA, 19 bp, Artificial Sequence
  Description of Artificial Sequence Synthetic primer
gaggaaagtccatgctcac

SEQ ID NO: 104 - DNA, 19 bp, Artificial Sequence
  Description of Artificial Sequence Synthetic primer
gaggaaagtccatgctcgc

SEQ ID NO: 105 - DNA, 17 bp, Artificial Sequence
  Description of Artificial Sequence Synthetic primer
gaggaaagtccgggctc

SEQ ID NO: 106 - DNA, 22 bp, Artificial Sequence
  Description of Artificial Sequence Synthetic primer
gataccctggtagtccacaccg

SEQ ID NO: 107 - DNA, 20 bp, Artificial Sequence
  Description of Artificial Sequence Synthetic primer
gatctggaggaataccggtg

SEQ ID NO: 108 - DNA, 29 bp, Artificial Sequence
  Description of Artificial Sequence Synthetic primer
gatgactttttagctaatggtcaggcagc

SEQ ID NO: 109 - DNA, 30 bp, Artificial Sequence
  Description of Artificial Sequence Synthetic primer
gattattgttatcctgttatgccatttgag

SEQ ID NO: 110 - DNA, 18 bp, Artificial Sequence
  Description of Artificial Sequence Synthetic primer
gcacaacctgcggctgcg

SEQ ID NO: 111 - DNA, 27 bp, Artificial Sequence
  Description of Artificial Sequence Synthetic primer
gcactatgcacacgtagattgtcctgg

SEQ ID NO: 112 - DNA, 21 bp, Artificial Sequence
  Description of Artificial Sequence Synthetic primer
gccttgtacacacctcccgtc

SEQ ID NO: 113 - DNA, 20 bp, Artificial Sequence
  Description of Artificial Sequence Synthetic primer
gcgaagaaccttaccaggtc

SEQ ID NO: 114 - DNA, 20 bp, Artificial Sequence
  Description of Artificial Sequence Synthetic primer
gctacacacgtgctacaatg

SEQ ID NO: 115 - DNA, 30 bp, Artificial Sequence
  Description of Artificial Sequence Synthetic primer
gctggtgaaaataacccagatgtcgtcttc

SEQ ID NO: 116 - DNA, 25 bp, Artificial Sequence
  Description of Artificial Sequence Synthetic primer
gcttcaggaatcaatgatggagcag

SEQ ID NO: 117 - DNA, 19 bp, Artificial Sequence
  Description of Artificial Sequence Synthetic primer
ggacggagaaggctatgtt

SEQ ID NO: 118 - DNA, 22 bp, Artificial Sequence
  Description of Artificial Sequence Synthetic primer
ggattagagaccctggtagtcc

SEQ ID NO: 119 - DNA, 26 bp, Artificial Sequence
  Description of Artificial Sequence Synthetic primer
ggattagataccctggtagtccacgc

SEQ ID NO: 120 - DNA, 25 bp, Artificial Sequence
  Description of Artificial Sequence Synthetic primer
ggctcagccatttagttaccgctat

SEQ ID NO: 121 - DNA, 21 bp, Artificial Sequence
  Description of Artificial Sequence Synthetic primer
gggaactgaaacatctaagta

SEQ ID NO: 122 - DNA, 22 bp, Artificial Sequence
  Description of Artificial Sequence Synthetic primer
gggagcaaacaggattagatac

SEQ ID NO: 123 - DNA, 29 bp, Artificial Sequence
  Description of Artificial Sequence Synthetic primer
gggcaacagcagcggattgcgattgcgcg

SEQ ID NO: 124 - DNA, 23 bp, Artificial Sequence
  Description of Artificial Sequence Synthetic primer
gggcagcgtttcggcgaaatgga

SEQ ID NO: 125 - DNA, 26 bp, Artificial Sequence
  Description of Artificial Sequence Synthetic primer
ggggagtgaaagagatcctgaaaccg

SEQ ID NO: 126 - DNA, 30 bp, Artificial Sequence
  Description of Artificial Sequence Synthetic primer
ggggattcagccatcaaagcagctattgac

SEQ ID NO: 127 - DNA, 27 bp, Artificial Sequence
  Description of Artificial Sequence Synthetic primer
ggggattgatatcaccgataagaagaa

SEQ ID NO: 128 - DNA, 24 bp, Artificial Sequence
  Description of Artificial Sequence Synthetic primer
ggtgaaagaagttgcctctaaagc

SEQ ID NO: 129 - DNA, 15 bp, Artificial Sequence
  Description of Artificial Sequence Synthetic primer
ggtggatgccttggc

SEQ ID NO: 130 - DNA, 20 bp, Artificial Sequence
  Description of Artificial Sequence Synthetic primer
ggtgttaaatagcctggcag

SEQ ID NO: 131 - DNA, 20 bp, Artificial Sequence
  Description of Artificial Sequence Synthetic primer
ggtttagtaccagaacatgc

SEQ ID NO: 132 - DNA, 23 bp, Artificial Sequence
  Description of Artificial Sequence Synthetic primer
gtcaaagtggcacgtttactggc

SEQ ID NO: 133 - DNA, 22 bp, Artificial Sequence
  Description of Artificial Sequence Synthetic primer
gtcgtgaaaacgagctggaaga

SEQ ID NO: 134 - DNA, 30 bp, Artificial Sequence
  Description of Artificial Sequence Synthetic primer
gtgagatgttgggttaagtcccgtaacgag

SEQ ID NO: 135 - DNA, 23 bp, Artificial Sequence
  Description of Artificial Sequence Synthetic primer
gtgcatgcggatacagagcagag

SEQ ID NO: 136 - DNA, 26 bp, Artificial Sequence
  Description of Artificial Sequence Synthetic primer
gtggcatgcctaatacatgcaagtcg

SEQ ID NO: 137 - DNA, 18 bp, Artificial Sequence
  Description of Artificial Sequence Synthetic primer
gtgtagcggtgaaatgcg

SEQ ID NO: 138 - DNA, 21 bp, Artificial Sequence
  Description of Artificial Sequence Synthetic primer
gttatcctgttatgccatttg

SEQ ID NO: 139 - DNA, 28 bp, Artificial Sequence
  Description of Artificial Sequence Synthetic primer
gttatttagcactcgtttttaatcagcc

SEQ ID NO: 140 - DNA, 22 bp, Artificial Sequence
  Description of Artificial Sequence Synthetic primer
gttgtgaggttaagcgactaag

SEQ ID NO: 141 - DNA, 22 bp, Artificial Sequence
  Description of Artificial Sequence Synthetic primer
gttgtgaggttaagcgactaag

SEQ ID NO: 142 - DNA, 24 bp, Artificial Sequence
  Description of Artificial Sequence Synthetic primer
tctagtaataataggaccctcagc

SEQ ID NO: 143 - DNA, 15 bp, Artificial Sequence
  Description of Artificial Sequence Synthetic primer
tatggctctactcaa

SEQ ID NO: 144 - DNA, 29 bp, Artificial Sequence
  Description of Artificial Sequence Synthetic primer
taaaacaaactacggtaacattgatcgca

SEQ ID NO: 145 - DNA, 33 bp, Artificial Sequence
  Description of Artificial Sequence Synthetic primer
taaaacttttgccgtaatgatgggtgaagatat

SEQ ID NO: 146 - DNA, 31 bp, Artificial Sequence
  Description of Artificial Sequence Synthetic primer
taaacacggctttcctatggcttatccaaat

SEQ ID NO: 147 - DNA, 28 bp, Artificial Sequence
  Description of Artificial Sequence Synthetic primer
taaaccccatcgggagcaagaccgaata

SEQ ID NO: 148 - DNA, 30 bp, Artificial Sequence
  Description of Artificial Sequence Synthetic primer
taaagcccgtgaaatgactcgtcgtaaagg

SEQ ID NO: 149 - DNA, 28 bp, Artificial Sequence
  Description of Artificial Sequence Synthetic primer
taaagttggttttattggttggcgcgga

SEQ ID NO: 150 - DNA, 25 bp, Artificial Sequence
  Description of Artificial Sequence Synthetic primer
taaatctgcccgtgtcgttggtgac

SEQ ID NO: 151 - DNA, 29 bp, Artificial Sequence
  Description of Artificial Sequence Synthetic primer
taacaactcgccttatgaaacgggatata

SEQ ID NO: 152 - DNA, 20 bp, Artificial Sequence
  Description of Artificial Sequence Synthetic primer
taacacatgcaagtcgaacg

SEQ ID NO: 153 - DNA, 29 bp, Artificial Sequence
  Description of Artificial Sequence Synthetic primer
taaccattcaagaactagatcttcaggca

SEQ ID NO: 154 - DNA, 30 bp, Artificial Sequence
  Description of Artificial Sequence Synthetic primer
taaccttaattggaaagaaacccaagaagt

SEQ ID NO: 155 - DNA, 25 bp, Artificial Sequence
  Description of Artificial Sequence Synthetic primer
taacggttatcatggcccagatggg

SEQ ID NO: 156 - DNA, 27 bp, Artificial Sequence
  Description of Artificial Sequence Synthetic primer
taactctgatgtttttgatgggaaggt

SEQ ID NO: 157 - DNA, 27 bp, Artificial Sequence
  Description of Artificial Sequence Synthetic primer
taactgcatggaacccttctttactag

SEQ ID NO: 158 - DNA, 27 bp, Artificial Sequence
  Description of Artificial Sequence Synthetic primer
taagaagccggaaaccatcaactaccg

SEQ ID NO: 159 - DNA, 22 bp, Artificial Sequence
  Description of Artificial Sequence Synthetic primer
taagagcgcaccggtaagttgg

SEQ ID NO: 160 - DNA, 28 bp, Artificial Sequence
  Description of Artificial Sequence Synthetic primer
taagcatgctgtggcttatcgtgaaatg

SEQ ID NO: 161 - DNA, 23 bp, Artificial Sequence
  Description of Artificial Sequence Synthetic primer
taagctgccagcggaatgctttc

SEQ ID NO: 162 - DNA, 29 bp, Artificial Sequence
  Description of Artificial Sequence Synthetic primer
taaggatagtgcaacagagatataccgcc

SEQ ID NO: 163 - DNA, 30 bp, Artificial Sequence
  Description of Artificial Sequence Synthetic primer
taaggtatgacaccggataaatcatataaa

SEQ ID NO: 164 - DNA, 33 bp, Artificial Sequence
  Description of Artificial Sequence Synthetic primer
taaggtttattgtctttgtggagatggggattt

SEQ ID NO: 165 - DNA, 31 bp, Artificial Sequence
  Description of Artificial Sequence Synthetic primer
taatcaagcattggaagatgaaatgcatacc

SEQ ID NO: 166 - DNA, 29 bp, Artificial Sequence
  Description of Artificial Sequence Synthetic primer
taatcggtaaatatcacccgcatggtgac

SEQ ID NO: 167 - DNA, 29 bp, Artificial Sequence
  Description of Artificial Sequence Synthetic primer
taatcggtaagtatcaccctcatggtgat

SEQ ID NO: 168 - DNA, 24 bp, Artificial Sequence
  Description of Artificial Sequence Synthetic primer
taatcgtggaatacgggtttgcta

SEQ ID NO: 169 - DNA, 30 bp, Artificial Sequence
  Description of Artificial Sequence Synthetic primer
taatgaaccctaatgaccatccacacggtg

SEQ ID NO: 170 - DNA, 27 bp, Artificial Sequence
  Description of Artificial Sequence Synthetic primer
taatgatgaattaggtgcgggttcttt

SEQ ID NO: 171 - DNA, 29 bp, Artificial Sequence
  Description of Artificial Sequence Synthetic primer
taatgggtaaatatcaccctcatggtgac

SEQ ID NO: 172 - DNA, 31 bp, Artificial Sequence
  Description of Artificial Sequence Synthetic primer
taattgggctctttctcgcttaaacacctta

SEQ ID NO: 173 - DNA, 25 bp, Artificial Sequence
  Description of Artificial Sequence Synthetic primer
tacaaagcaagacactggctcacta

SEQ ID NO: 174 - DNA, 30 bp, Artificial Sequence
  Description of Artificial Sequence Synthetic primer
tacaaaggtcaaccaatgacattcagacta

SEQ ID NO: 175 - DNA, 34 bp, Artificial Sequence
  Description of Artificial Sequence Synthetic primer
tacaacatattattaaagagacgggtttgaatcc

SEQ ID NO: 176 - DNA, 20 bp, Artificial Sequence
  Description of Artificial Sequence Synthetic primer
tacaagcactcccagctgca

SEQ ID NO: 177 - DNA, 29 bp, Artificial Sequence
  Description of Artificial Sequence Synthetic primer
tacaatgcttgtttatgctggtaaagcag

SEQ ID NO: 178 - DNA, 25 bp, Artificial Sequence
  Description of Artificial Sequence Synthetic primer
tacacaacaatggcggtaaagatgg

SEQ ID NO: 179 - DNA, 15 bp, Artificial Sequence
  Description of Artificial Sequence Synthetic primer
tacagagtttgcgac

SEQ ID NO: 180 - DNA, 21 bp, Artificial Sequence
  Description of Artificial Sequence Synthetic primer
tacaggccgtgttgaacgtgg

SEQ ID NO: 181 - DNA, 21 bp, Artificial Sequence
  Description of Artificial Sequence Synthetic primer
tacatgctagccgcgtcttac

SEQ ID NO: 182 - DNA, 27 bp, Artificial Sequence
  Description of Artificial Sequence Synthetic primer
taccactattaatgtcgctggtgcttc

SEQ ID NO: 183 - DNA, 25 bp, Artificial Sequence
  Description of Artificial Sequence Synthetic primer
taccatgacagaaggcattttgaca

SEQ ID NO: 184 - DNA, 19 bp, Artificial Sequence
  Description of Artificial Sequence Synthetic primer
taccccaaaccgacacagg

SEQ ID NO: 185 - DNA, 23 bp, Artificial Sequence
  Description of Artificial Sequence Synthetic primer
taccccagggaaagtgccacaga

SEQ ID NO: 186 - DNA, 25 bp, Artificial Sequence
  Description of Artificial Sequence Synthetic primer
taccggcgcaaaaagtcgagattgg

SEQ ID NO: 187 - DNA, 21 bp, Artificial Sequence
  Description of Artificial Sequence Synthetic primer
tacctatatgcgccagaccgc

SEQ ID NO: 188 - DNA, 26 bp, Artificial Sequence
  Description of Artificial Sequence Synthetic primer
tacgatttcacttccgcagccagatt

SEQ ID NO: 189 - DNA, 27 bp, Artificial Sequence
  Description of Artificial Sequence Synthetic primer
tacgcgtcttgaagcgtttcgttatga

SEQ ID NO: 190 - DNA, 26 bp, Artificial Sequence
  Description of Artificial Sequence Synthetic primer
tacgctgacggaatcaaccaaagcgg

SEQ ID NO: 191 - DNA, 21 bp, Artificial Sequence
  Description of Artificial Sequence Synthetic primer
tacggtgaatacgttcccggg

SEQ ID NO: 192 - DNA, 15 bp, Artificial Sequence
  Description of Artificial Sequence Synthetic primer
tacagagtttgcgac

SEQ ID NO: 193 - DNA, 23 bp, Artificial Sequence
  Description of Artificial Sequence Synthetic primer
tactacttcaagccgaacttccg

SEQ ID NO: 194 - DNA, 28 bp, Artificial Sequence
  Description of Artificial Sequence Synthetic primer
tactagcggtaagcttaaacaagattgc

SEQ ID NO: 195 - DNA, 22 bp, Artificial Sequence
  Description of Artificial Sequence Synthetic primer
tactctcggtggagaagctcgc

SEQ ID NO: 196 - DNA, 22 bp, Artificial Sequence
  Description of Artificial Sequence Synthetic primer
tactggaacaaagtctgcgacc

SEQ ID NO: 197 - DNA, 27 bp, Artificial Sequence
  Description of Artificial Sequence Synthetic primer
tacttactacttcaagccgaacttccg

SEQ ID NO: 198 - DNA, 28 bp, Artificial Sequence
  Description of Artificial Sequence Synthetic primer
tacttacttgagaatccacaagctgcaa

SEQ ID NO: 199 - DNA, 28 bp, Artificial Sequence
  Description of Artificial Sequence Synthetic primer
tacttggtaaataccacccacatggtga

SEQ ID NO: 200 - DNA, 32 bp, Artificial Sequence
  Description of Artificial Sequence Synthetic primer
tacttttttaaaactagggatgcgtttgaagc

SEQ ID NO: 201 - DNA, 27 bp, Artificial Sequence
  Description of Artificial Sequence Synthetic primer
tagaaatcaaggtgatagtggcaatga

SEQ ID NO: 202 - DNA, 21 bp, Artificial Sequence
  Description of Artificial Sequence Synthetic primer
tagaacaccgatggcgaaggc

SEQ ID NO: 203 - DNA, 22 bp, Artificial Sequence
  Description of Artificial Sequence Synthetic primer
tagaacgtcgcgagacagttcg

SEQ ID NO: 204 - DNA, 21 bp, Artificial Sequence
  Description of Artificial Sequence Synthetic primer
tagactgcccaggacacgctg

SEQ ID NO: 205 - DNA, 29 bp, Artificial Sequence
  Description of Artificial Sequence Synthetic primer
tagataattgggctctttctcgcttaaac

SEQ ID NO: 206 - DNA, 22 bp, Artificial Sequence
  Description of Artificial Sequence Synthetic primer
tagataccctggtagtccacgc

SEQ ID NO: 207 - DNA, 28 bp, Artificial Sequence
  Description of Artificial Sequence Synthetic primer
tagatgaaaaaggcgaagtggctaatgg

SEQ ID NO: 208 - DNA, 28 bp, Artificial Sequence
  Description of Artificial Sequence Synthetic primer
tagatgaaaagggcgaagtggctaatgg

SEQ ID NO: 209 - DNA, 30 bp, Artificial Sequence
  Description of Artificial Sequence Synthetic primer
tagcaacaaatatatctgaagcagcgtact

SEQ ID NO: 210 - DNA, 29 bp, Artificial Sequence
  Description of Artificial Sequence Synthetic primer
tagcaggtggtgaaatcggccacatgatt

SEQ ID NO: 211 - DNA, 26 bp, Artificial Sequence
  Description of Artificial Sequence Synthetic primer
tagcatcagaactgttgttccgctag

SEQ ID NO: 212 - DNA, 24 bp, Artificial Sequence
  Description of Artificial Sequence Synthetic primer
tagcccagcacaatttgtgattca

SEQ ID NO: 213 - DNA, 34 bp, Artificial Sequence
  Description of Artificial Sequence Synthetic primer
tagcctttaacgaaaatgtaaaaatgcgttttga

SEQ ID NO: 214 - DNA, 27 bp, Artificial Sequence
  Description of Artificial Sequence Synthetic primer
tagcgaatgtggctttacttcacaatt

SEQ ID NO: 215 - DNA, 19 bp, Artificial Sequence
  Description of Artificial Sequence Synthetic primer
tagcgtaaaggtgaacctt

SEQ ID NO: 216 - DNA, 20 bp, Artificial Sequence
  Description of Artificial Sequence Synthetic primer
tagctaatggtcaggcagcc

SEQ ID NO: 217 - DNA, 30 bp, Artificial Sequence
  Description of Artificial Sequence Synthetic primer
tagctatcttatcgttgagaagggatttgc

SEQ ID NO: 218 - DNA, 22 bp, Artificial Sequence
  Description of Artificial Sequence Synthetic primer
tagctggcgcgaaattaggtgt

SEQ ID NO: 219 - DNA, 26 bp, Artificial Sequence
  Description of Artificial Sequence Synthetic primer
tagctggcggtatggagaatatgtct

SEQ ID NO: 220 - DNA, 27 bp, Artificial Sequence
  Description of Artificial Sequence Synthetic primer
tagcttttgcatattatatcgagccac

SEQ ID NO: 221 - DNA, 29 bp, Artificial Sequence
  Description of Artificial Sequence Synthetic primer
taggaattacggctgataaagcgtataaa

SEQ ID NO: 222 - DNA, 35 bp, Artificial Sequence
  Description of Artificial Sequence Synthetic primer
taggcgaagatatacaaagagtattagaagctaga

SEQ ID NO: 223 - DNA, 25 bp, Artificial Sequence
  Description of Artificial Sequence Synthetic primer
taggcgtgaaagcaagctaccgttt

SEQ ID NO: 224 - DNA, 25 bp, Artificial Sequence
  Description of Artificial Sequence Synthetic primer
taggtgctggttacgcagatcaaga

SEQ ID NO: 225 - DNA, 30 bp, Artificial Sequence
  Description of Artificial Sequence Synthetic primer
taggtttacgtcagtatggcgtgattatgg

SEQ ID NO: 226 - DNA, 23 bp, Artificial Sequence
  Description of Artificial Sequence Synthetic primer
tagtaccgaagctggtcatacga

SEQ ID NO: 227 - DNA, 17 bp, Artificial Sequence
  Description of Artificial Sequence Synthetic primer
tagtacgagaggaccgg

SEQ ID NO: 228 - DNA, 18 bp, Artificial Sequence
  Description of Artificial Sequence Synthetic primer
tagtcccgcaacgagcgc

SEQ ID NO: 229 - DNA, 27 bp, Artificial Sequence
  Description of Artificial Sequence Synthetic primer
tagtgatagaactgtaggcacaatcgt

SEQ ID NO: 230 - DNA, 22 bp, Artificial Sequence
  Description of Artificial Sequence Synthetic primer
tagttgctcaaacagctgggct

SEQ ID NO: 231 - DNA, 29 bp, Artificial Sequence
  Description of Artificial Sequence Synthetic primer
tataagtgggtaaaccgtgaatatcgtgt

SEQ ID NO: 232 - DNA, 24 bp, Artificial Sequence
  Description of Artificial Sequence Synthetic primer
tatacttcaacgcctgctgctttc

SEQ ID NO: 233 - DNA, 22 bp, Artificial Sequence
  Description of Artificial Sequence Synthetic primer
tatcgctcaggcgaactccaac

SEQ ID NO: 234 - DNA, 23 bp, Artificial Sequence
  Description of Artificial Sequence Synthetic primer
tatgaccaaactcatcagacgag

SEQ ID NO: 235 - DNA, 30 bp, Artificial Sequence
  Description of Artificial Sequence Synthetic primer
tatgattacaattcaagaaggtcgtcacgc

SEQ ID NO: 236 - DNA, 27 bp, Artificial Sequence
  Description of Artificial Sequence Synthetic primer
tatgcagtggaacgatggtttccaaga

SEQ ID NO: 237 - DNA, 24 bp, Artificial Sequence
  Description of Artificial Sequence Synthetic primer
tatgctgaccgaccagtggtacgt

SEQ ID NO: 238 - DNA, 21 bp, Artificial Sequence
  Description of Artificial Sequence Synthetic primer
tatggccatggcagaagctca

SEQ ID NO: 239 - DNA, 15 bp, Artificial Sequence
  Description of Artificial Sequence Synthetic primer
tatggctctactcaa

SEQ ID NO: 240 - DNA, 31 bp, Artificial Sequence
  Description of Artificial Sequence Synthetic primer
tatgtccaagaagcatagcaaaaaaagcaat

SEQ ID NO: 241 - DNA, 27 bp, Artificial Sequence
  Description of Artificial Sequence Synthetic primer
tattcaaggtggtcctttgatgcatgt

SEQ ID NO: 242 - DNA, 22 bp, Artificial Sequence
  Description of Artificial Sequence Synthetic primer
tattggacaacggtcgtcgcgg

SEQ ID NO: 243 - DNA, 30 bp, Artificial Sequence
  Description of Artificial Sequence Synthetic primer
tattgtttcaaatgtacaaggtgaagtgcg

SEQ ID NO: 244 - DNA, 30 bp, Artificial Sequence
  Description of Artificial Sequence Synthetic primer
tatttcacatgtaattttgatattcgcact

SEQ ID NO: 245 - DNA, 32 bp, Artificial Sequence
  Description of Artificial Sequence Synthetic primer
tcaaaaagccctaggtaaagagattccatatc

SEQ ID NO: 246 - DNA, 29 bp, Artificial Sequence
  Description of Artificial Sequence Synthetic primer
tcaaactgggcaatcggaactggtaaatc

SEQ ID NO: 247 - DNA, 26 bp, Artificial Sequence
  Description of Artificial Sequence Synthetic primer
tcaaatgtacaaggtgaagtgcgtga

SEQ ID NO: 248 - DNA, 29 bp, Artificial Sequence
  Description of Artificial Sequence Synthetic primer
tcaacaacctcttggaggtaaagctcagt

SEQ ID NO: 249 - DNA, 19 bp, Artificial Sequence
  Description of Artificial Sequence Synthetic primer
tcaacctcggcccgaacca

SEQ ID NO: 250 - DNA, 25 bp, Artificial Sequence
  Description of Artificial Sequence Synthetic primer
tcaacctgactgcgtgaatggttgt

SEQ ID NO: 251 - DNA, 27 bp, Artificial Sequence
  Description of Artificial Sequence Synthetic primer
tcaacgaaggtaaaaaccatctcaacg

SEQ ID NO: 252 - DNA, 27 bp, Artificial Sequence
  Description of Artificial Sequence Synthetic primer
tcaacggtaacttctatgttacttctg

SEQ ID NO: 253 - DNA, 26 bp, Artificial Sequence
  Description of Artificial Sequence Synthetic primer
tcaactcgaattttcaacaggtacca

SEQ ID NO: 254 - DNA, 17 bp, Artificial Sequence
  Description of Artificial Sequence Synthetic primer
tcaagaagaaaaagagc

SEQ ID NO: 255 - DNA, 24 bp, Artificial Sequence
  Description of Artificial Sequence Synthetic primer
tcaagcaaacgcacaatcagaagc

SEQ ID NO: 256 - DNA, 27 bp, Artificial Sequence
  Description of Artificial Sequence Synthetic primer
tcaagcagaagctttggaagaagaagg

SEQ ID NO: 257 - DNA, 27 bp, Artificial Sequence
  Description of Artificial Sequence Synthetic primer
tcaagccgtacgtattattaggtgctg

SEQ ID NO: 258 - DNA, 27 bp, Artificial Sequence
  Description of Artificial Sequence Synthetic primer
tcaataccgcaacagcggtggcttggg

SEQ ID NO: 259 - DNA, 28 bp, Artificial Sequence
  Description of Artificial Sequence Synthetic primer
tcaatgaacgaccaacaagtgattgatg

SEQ ID NO: 260 - DNA, 28 bp, Artificial Sequence
  Description of Artificial Sequence Synthetic primer
tcaatgaacgatcaacaagtgattgatg

SEQ ID NO: 261 - DNA, 24 bp, Artificial Sequence
  Description of Artificial Sequence Synthetic primer
tcacatatcgtgagcaatgaactg

SEQ ID NO: 262 - DNA, 30 bp, Artificial Sequence
  Description of Artificial Sequence Synthetic primer
tcaccaggttcaactcaaaaaatattaaca

SEQ ID NO: 263 - DNA, 25 bp, Artificial Sequence
  Description of Artificial Sequence Synthetic primer
tcaccagtttgccacgtatcttcaa

SEQ ID NO: 264 - DNA, 28 bp, Artificial Sequence
  Description of Artificial Sequence Synthetic primer
tcaccctcatggtgactcatctatttat

SEQ ID NO: 265 - DNA, 28 bp, Artificial Sequence
  Description of Artificial Sequence Synthetic primer
tcaccctcatggtgattcagctgtttat

SEQ ID NO: 266 - DNA, 25 bp, Artificial Sequence
  Description of Artificial Sequence Synthetic primer
tcaccgatatcatggcttaccacgg

SEQ ID NO: 267 - DNA, 23 bp, Artificial Sequence
  Description of Artificial Sequence Synthetic primer
tcaccgtgccgttcaaggaagag

SEQ ID NO: 268 - DNA, 29 bp, Artificial Sequence
  Description of Artificial Sequence Synthetic primer
tcacctccaagtttagatcacttgagaga

SEQ ID NO: 269 - DNA, 26 bp, Artificial Sequence
  Description of Artificial Sequence Synthetic primer
tcacgataagaaaaccggtcaagagg

SEQ ID NO: 270 - DNA, 27 bp, Artificial Sequence
  Description of Artificial Sequence Synthetic primer
tcactcttacatataaggaaggcgctc

SEQ ID NO: 271 - DNA, 25 bp, Artificial Sequence
  Description of Artificial Sequence Synthetic primer
tcagaccatgctcgcagagaaactt

SEQ ID NO: 272 - DNA, 25 bp, Artificial Sequence
  Description of Artificial Sequence Synthetic primer
tcagagaccgttttatcctatcagc

SEQ ID NO: 273 - DNA, 26 bp, Artificial Sequence
  Description of Artificial Sequence Synthetic primer
tcagcaaatgcatcacaaacagataa

SEQ ID NO: 274 - DNA, 25 bp, Artificial Sequence
  Description of Artificial Sequence Synthetic primer
tcagcatatgcacatggaacacctc

SEQ ID NO: 275 - DNA, 26 bp, Artificial Sequence
  Description of Artificial Sequence Synthetic primer
tcagcatatgcacatggaacacctca

SEQ ID NO: 276 - DNA, 22 bp, Artificial Sequence
  Description of Artificial Sequence Synthetic primer
tcagccatcaaagcagctattg

SEQ ID NO: 277 - DNA, 21 bp, Artificial Sequence
  Description of Artificial Sequence Synthetic primer
tcagcgcgtacagtgggtgat

SEQ ID NO: 278 - DNA, 25 bp, Artificial Sequence
  Description of Artificial Sequence Synthetic primer
tcagcgtatgcacatggaactcctc

SEQ ID NO: 279 - DNA, 23 bp, Artificial Sequence
  Description of Artificial Sequence Synthetic primer
tcagctacatcgactatgcgatg

SEQ ID NO: 280 - DNA, 30 bp, Artificial Sequence
  Description of Artificial Sequence Synthetic primer
tcagctagaccttttaggtaaagctaagct

SEQ ID NO: 281 - DNA, 29 bp, Artificial Sequence
  Description of Artificial Sequence Synthetic primer
tcagctatttttccaggtatccaaggtgg

SEQ ID NO: 282 - DNA, 23 bp, Artificial Sequence
  Description of Artificial Sequence Synthetic primer
tcagctgtcgcagttcatggacc

SEQ ID NO: 283 - DNA, 25 bp, Artificial Sequence
  Description of Artificial Sequence Synthetic primer
tcaggaaaagggcattttacccttg

SEQ ID NO: 284 - DNA, 30 bp, Artificial Sequence
  Description of Artificial Sequence Synthetic primer
tcaggagtcgttcaactcgatctacatgat

SEQ ID NO: 285 - DNA, 31 bp, Artificial Sequence
  Description of Artificial Sequence Synthetic primer
tcaggagtcgttcaactcgatctacatgatg

SEQ ID NO: 286 - DNA, 30 bp, Artificial Sequence
  Description of Artificial Sequence Synthetic primer
tcaggatggaaataaccaccaattcactac

SEQ ID NO: 287 - DNA, 23 bp, Artificial Sequence
  Description of Artificial Sequence Synthetic primer
tcaggcattgcggttgggatggc

SEQ ID NO: 288 - DNA, 24 bp, Artificial Sequence
  Description of Artificial Sequence Synthetic primer
tcaggtactgctatccaccctcaa

SEQ ID NO: 289 - DNA, 22 bp, Artificial Sequence
  Description of Artificial Sequence Synthetic primer
tcaggtggcttacacggcgtag

SEQ ID NO: 290 - DNA, 26 bp, Artificial Sequence
  Description of Artificial Sequence Synthetic primer
tcagtatgtatccaccgtagccagtc

SEQ ID NO: 291 - DNA, 23 bp, Artificial Sequence
  Description of Artificial Sequence Synthetic primer
tcagttccgttatcgccattgca

SEQ ID NO: 292 - DNA, 24 bp, Artificial Sequence
  Description of Artificial Sequence Synthetic primer
tcagttccgttatcgccattgcat

SEQ ID NO: 293 - DNA, 25 bp, Artificial Sequence
  Description of Artificial Sequence Synthetic primer
tcagttccgttatcgccattgcatt

SEQ ID NO: 294 - DNA, 24 bp, Artificial Sequence
  Description of Artificial Sequence Synthetic primer
tcagttcggcggtcagcgcttcgg

SEQ ID NO: 295 - DNA, 34 bp, Artificial Sequence
  Description of Artificial Sequence Synthetic primer
tcagttttaatgtctcgtatgatcgaatcaaaag

SEQ ID NO: 296 - DNA, 24 bp, Artificial Sequence
  Description of Artificial Sequence Synthetic primer
tcatccacacggtggtggtgaagg

SEQ ID NO: 297 - DNA, 28 bp, Artificial Sequence
  Description of Artificial Sequence Synthetic primer
tcatcctaagccaagtgtagactctgta

SEQ ID NO: 298 - DNA, 31 bp, Artificial Sequence
  Description of Artificial Sequence Synthetic primer
tcatgataatatctttgaaatcggctcagga

SEQ ID NO: 299 - DNA, 33 bp, Artificial Sequence
  Description of Artificial Sequence Synthetic primer
tcatgttgagcttaaacctatagaagtaaaagc

SEQ ID NO: 300 - DNA, 27 bp, Artificial Sequence
  Description of Artificial Sequence Synthetic primer
tcattatcatgcgccaatgagtgcaga

SEQ ID NO: 301 - DNA, 28 bp, Artificial Sequence
  Description of Artificial Sequence Synthetic primer
tcattcaagaactagatcttcaggcaag

SEQ ID NO: 302 - DNA, 26 bp, Artificial Sequence
  Description of Artificial Sequence Synthetic primer
tccaaaaaaatcagcgcgtacagtgg

SEQ ID NO: 303 - DNA, 29 bp, Artificial Sequence
  Description of Artificial Sequence Synthetic primer
tccaaaccaggtgtatcaagaacatcagg

SEQ ID NO: 304 - DNA, 29 bp, Artificial Sequence
  Description of Artificial Sequence Synthetic primer
tccaaataagtggcgttacaaatactgaa

SEQ ID NO: 305 - DNA, 32 bp, Artificial Sequence
  Description of Artificial Sequence Synthetic primer
tccaacgaagtacaatacaagacaaaagaagg

SEQ ID NO: 306 - DNA, 32 bp, Artificial Sequence
  Description of Artificial Sequence Synthetic primer
tccaaggtacactaaacttacttgagctaatg

SEQ ID NO: 307 - DNA, 24 bp, Artificial Sequence
  Description of Artificial Sequence Synthetic primer
tccaatgccacaaactcgtgaaca

SEQ ID NO: 308 - DNA, 24 bp, Artificial Sequence
  Description of Artificial Sequence Synthetic primer
tccacacgccgttcttcaacaact

SEQ ID NO: 309 - DNA, 21 bp, Artificial Sequence
  Description of Artificial Sequence Synthetic primer
tccacacggtggtggtgaagg

SEQ ID NO: 310 - DNA, 26 bp, Artificial Sequence
  Description of Artificial Sequence Synthetic primer
tccaccaagagcaagatcaaataggc

SEQ ID NO: 311 - DNA, 20 bp, Artificial Sequence
  Description of Artificial Sequence Synthetic primer
tccacggtcatggagcgcta

SEQ ID NO: 312 - DNA, 30 bp, Artificial Sequence
  Description of Artificial Sequence Synthetic primer
tccacttatcgcaaatggaaaattaagcaa

SEQ ID NO: 313 - DNA, 33 bp, Artificial Sequence
  Description of Artificial Sequence Synthetic primer
tccagatggacaaattttcttagaaactgattt

SEQ ID NO: 314 - DNA, 26 bp, Artificial Sequence
  Description of Artificial Sequence Synthetic primer
tccagcacgaattgctgctatgaaag

SEQ ID NO: 315 - DNA, 33 bp, Artificial Sequence
  Description of Artificial Sequence Synthetic primer
tccaggacaaatgtatgaaaaatgtccaagaag

SEQ ID NO: 316 - DNA, 25 bp, Artificial Sequence
  Description of Artificial Sequence Synthetic primer
tccattgttcgtatggctcaagact

SEQ ID NO: 317 - DNA, 31 bp, Artificial Sequence
  Description of Artificial Sequence Synthetic primer
tcccaattaattctgccatttttccaggtat

SEQ ID NO: 318 - DNA, 34 bp, Artificial Sequence
  Description of Artificial Sequence Synthetic primer
tcccacgaaacagatgaagaaattaacaaaaaag

SEQ ID NO: 319 - DNA, 30 bp, Artificial Sequence
  Description of Artificial Sequence Synthetic primer
tcccagctagaccttttaggtaaagctaag

SEQ ID NO: 320 - DNA, 26 bp, Artificial Sequence
  Description of Artificial Sequence Synthetic primer
tcccaggtgacgatgtacctgtaatc

SEQ ID NO: 321 - DNA, 26 bp, Artificial Sequence
  Description of Artificial Sequence Synthetic primer
tccccaggacaccctgaaatttcaac

SEQ ID NO: 322 - DNA, 34 bp, Artificial Sequence
  Description of Artificial Sequence Synthetic primer
tcccccacgctttaattgtttatgatgatttgag

SEQ ID NO: 323 - DNA, 31 bp, Artificial Sequence
  Description of Artificial Sequence Synthetic primer
tcccggacttaatatcaatgaaaattgtgga

SEQ ID NO: 324 - DNA, 28 bp, Artificial Sequence
  Description of Artificial Sequence Synthetic primer
tcccggagcttttatgactaaagcagat

SEQ ID NO: 325 - DNA, 19 bp, Artificial Sequence
  Description of Artificial Sequence Synthetic primer
tccgcggagttgactgggt

SEQ ID NO: 326 - DNA, 20 bp, Artificial Sequence
  Description of Artificial Sequence Synthetic primer
tccgctgaatctgtcgccgc

SEQ ID NO: 327 - DNA, 23 bp, Artificial Sequence
  Description of Artificial Sequence Synthetic primer
tccggctcacgttattatggtac

SEQ ID NO: 328 - DNA, 27 bp, Artificial Sequence
  Description of Artificial Sequence Synthetic primer
tccgtacgtattattaggtgctggtca

SEQ ID NO: 329 - DNA, 30 bp, Artificial Sequence
  Description of Artificial Sequence Synthetic primer
tccgttatcgccattgcattatttggaact

SEQ ID NO: 330 - DNA, 33 bp, Artificial Sequence
  Description of Artificial Sequence Synthetic primer
tccgttcttacaaatagcaatagaacttgaagc

SEQ ID NO: 331 - DNA, 36 bp, Artificial Sequence
  Description of Artificial Sequence Synthetic primer
tccgttgattattgttatcctgttatgccatttgag

SEQ ID NO: 332 - DNA, 34 bp, Artificial Sequence
  Description of Artificial Sequence Synthetic primer
tcctaatggacttaatatcaatgaaaattgtgga

SEQ ID NO: 333 - DNA, 22 bp, Artificial Sequence
  Description of Artificial Sequence Synthetic primer
tcctagaggaatggctgccacg

SEQ ID NO: 334 - DNA, 30 bp, Artificial Sequence
  Description of Artificial Sequence Synthetic primer
tcctatattaatcgtttacagaaactggct

SEQ ID NO: 335 - DNA, 31 bp, Artificial Sequence
  Description of Artificial Sequence Synthetic primer
tcctcaatgaacgancaacaagtgattgatg

SEQ ID NO: 336 - DNA, 31 bp, Artificial Sequence
  Description of Artificial Sequence Synthetic primer
tcctcaatgaatgatcaacaagtgattgatg

SEQ ID NO: 337 - DNA, 31 bp, Artificial Sequence
  Description of Artificial Sequence Synthetic primer
tcctcgatgaacgancaacaagtnattgatg

SEQ ID NO: 338 - DNA, 31 bp, Artificial Sequence
  Description of Artificial Sequence Synthetic primer
tcctcgatgaatgancaacaagtnattgatg

SEQ ID NO: 339 - DNA, 31 bp, Artificial Sequence
  Description of Artificial Sequence Synthetic primer
tcctcnatgaacgancaacaagtgattgatg

SEQ ID NO: 340 - DNA, 31 bp, Artificial Sequence
  Description of Artificial Sequence Synthetic primer
tcctcnatgaangancaacaagtnattgatg

SEQ ID NO: 341 - DNA, 20 bp, Artificial Sequence
  Description of Artificial Sequence Synthetic primer
tcctgaaaaatggagcacgg

SEQ ID NO: 342 - DNA, 23 bp, Artificial Sequence
  Description of Artificial Sequence Synthetic primer
tcctgaagcaagtgcatttacga

SEQ ID NO: 343 - DNA, 28 bp, Artificial Sequence
  Description of Artificial Sequence Synthetic primer
tcctgaccgacccattattccctttatc

SEQ ID NO: 344 - DNA, 33 bp, Artificial Sequence
  Description of Artificial Sequence Synthetic primer
tcctgatgctcaaagtgcttttttagatccttt

SEQ ID NO: 345 - DNA, 34 bp, Artificial Sequence
  Description of Artificial Sequence Synthetic primer
tcctgttatccctgaagtagttaatcaagtttgt

SEQ ID NO: 346 - DNA, 35 bp, Artificial Sequence
  Description of Artificial Sequence Synthetic primer
tcctgttatccctgaagtagttaatcaagtttgtt

SEQ ID NO: 347 - DNA, 36 bp, Artificial Sequence
  Description of Artificial Sequence Synthetic primer
tcctgttattcctgaagtagttaatcaagtttgtta

SEQ ID NO: 348 - DNA, 31 bp, Artificial Sequence
  Description of Artificial Sequence Synthetic primer
tccttacttcgaactatgaatcttttggaag

SEQ ID NO: 349 - DNA, 29 bp, Artificial Sequence
  Description of Artificial Sequence Synthetic primer
tccttatagggatggctatcagtaatgtt

SEQ ID NO: 350 - DNA, 22 bp, Artificial Sequence
  Description of Artificial Sequence Synthetic primer
tccttgaccgcctttccgatac

SEQ ID NO: 351 - DNA, 33 bp, Artificial Sequence
  Description of Artificial Sequence Synthetic primer
tccttgctttagttttaagtgcatgtaattcaa

SEQ ID NO: 352 - DNA, 32 bp, Artificial Sequence
  Description of Artificial Sequence Synthetic primer
tcctttgatatattatgcgatggaaggttggt

SEQ ID NO: 353 - DNA, 30 bp, Artificial Sequence
  Description of Artificial Sequence Synthetic primer
tcctttgatgcatgtaattgctgcaaaagc

SEQ ID NO: 354 - DNA, 31 bp, Artificial Sequence
  Description of Artificial Sequence Synthetic primer
tcgaaagcttttgcatattatatcgagccac

SEQ ID NO: 355 - DNA, 28 bp, Artificial Sequence
  Description of Artificial Sequence Synthetic primer
tcgaagtacaatacaagacaaaagaagg

SEQ ID NO: 356 - DNA, 28 bp, Artificial Sequence
  Description of Artificial Sequence Synthetic primer
tcgacaacaccattatctatggtgtgaa

SEQ ID NO: 357 - DNA, 23 bp, Artificial Sequence
  Description of Artificial Sequence Synthetic primer
tcgacctttggcaggaactagac

SEQ ID NO: 358 - DNA, 17 bp, Artificial Sequence
  Description of Artificial Sequence Synthetic primer
tcgagcaggcgctgccg

SEQ ID NO: 359 - DNA, 31 bp, Artificial Sequence
  Description of Artificial Sequence Synthetic primer
tcgagtatagctaaaaaaatagtttatgaca

SEQ ID NO: 360 - DNA, 26 bp, Artificial Sequence
  Description of Artificial Sequence Synthetic primer
tcgatctggtttcatgctgtttcagt

SEQ ID NO: 361 - DNA, 28 bp, Artificial Sequence
  Description of Artificial Sequence Synthetic primer
tcgatgaacgaccaacaagtgattgatg

SEQ ID NO: 362 - DNA, 24 bp, Artificial Sequence
  Description of Artificial Sequence Synthetic primer
tcgattaggcagcaacgaaagccg

SEQ ID NO: 363 - DNA, 24 bp, Artificial Sequence
  Description of Artificial Sequence Synthetic primer
tcgcaaaaaaatccagctattagc

SEQ ID NO: 364 - DNA, 26 bp, Artificial Sequence
  Description of Artificial Sequence Synthetic primer
tcgccaatcaaaactaagggaatggc

SEQ ID NO: 365 - DNA, 19 bp, Artificial Sequence
  Description of Artificial Sequence Synthetic primer
tcgccatcgtcaccaaccg

SEQ ID NO: 366 - DNA, 16 bp, Artificial Sequence
  Description of Artificial Sequence Synthetic primer
tcgcccgcgaggacgt

SEQ ID NO: 367 - DNA, 21 bp, Artificial Sequence
  Description of Artificial Sequence Synthetic primer
tcgccgacttcgacggtgacc

SEQ ID NO: 368 - DNA, 22 bp, Artificial Sequence
  Description of Artificial Sequence Synthetic primer
tcgccggcaatgccattggata

SEQ ID NO: 369 - DNA, 23 bp, Artificial Sequence
  Description of Artificial Sequence Synthetic primer
tcgccgtggaaaaatcctacgct

SEQ ID NO: 370 - DNA, 31 bp, Artificial Sequence
  Description of Artificial Sequence Synthetic primer
tcgcgttgcaacaaaactttctaaagtatgt

SEQ ID NO: 371 - DNA, 24 bp, Artificial Sequence
  Description of Artificial Sequence Synthetic primer
tcgctacaggccctttaggacaag

SEQ ID NO: 372 - DNA, 27 bp, Artificial Sequence
  Description of Artificial Sequence Synthetic primer
tcgctatcttatcgttgagaagggatt

SEQ ID NO: 373 - DNA, 24 bp, Artificial Sequence
  Description of Artificial Sequence Synthetic primer
tcggaatctgatgttgcagttgtt

SEQ ID NO: 374 - DNA, 22 bp, Artificial Sequence
  Description of Artificial Sequence Synthetic primer
tcggccgcaccttcatcgaagt

SEQ ID NO: 375 - DNA, 28 bp, Artificial Sequence
  Description of Artificial Sequence Synthetic primer
tcggcgaaatccgtattcctgaaaatga

SEQ ID NO: 376 - DNA, 18 bp, Artificial Sequence
  Description of Artificial Sequence Synthetic primer
tcggcgcctgcctgatga

SEQ ID NO: 377 - DNA, 23 bp, Artificial Sequence
  Description of Artificial Sequence Synthetic primer
tcgggtgatgatgcgcgtgaagg

SEQ ID NO: 378 - DNA, 31 bp, Artificial Sequence
  Description of Artificial Sequence Synthetic primer
tcggtttagtaaaagaacgtattgctcaacc

SEQ ID NO: 379 - DNA, 28 bp, Artificial Sequence
  Description of Artificial Sequence Synthetic primer
tcgtacgtattattaggtgctggtcact

SEQ ID NO: 380 - DNA, 21 bp, Artificial Sequence
  Description of Artificial Sequence Synthetic primer
tcgtatggctcaatggtggag

SEQ ID NO: 381 - DNA, 30 bp, Artificial Sequence
  Description of Artificial Sequence Synthetic primer
tcgtctttttgattctttccctgataatgc

SEQ ID NO: 382 - DNA, 32 bp, Artificial Sequence
  Description of Artificial Sequence Synthetic primer
tcgtctttttgattctttccctgataatgctc

SEQ ID NO: 383 - DNA, 25 bp, Artificial Sequence
  Description of Artificial Sequence Synthetic primer
tcgtgattatggatggcaacgtgaa

SEQ ID NO: 384 - DNA, 24 bp, Artificial Sequence
  Description of Artificial Sequence Synthetic primer
tcgtgcccgcaatttgcataaagc

SEQ ID NO: 385 - DNA, 21 bp, Artificial Sequence
  Description of Artificial Sequence Synthetic primer
tcgtggcggcgtggttatcga

SEQ ID NO: 386 - DNA, 27 bp, Artificial Sequence
  Description of Artificial Sequence Synthetic primer
tcgtgttgaacgtggtcaaatcaaagt

SEQ ID NO: 387 - DNA, 23 bp, Artificial Sequence
  Description of Artificial Sequence Synthetic primer
tcgttcctggaacacgatgacgc

SEQ ID NO: 388 - DNA, 27 bp, Artificial Sequence
  Description of Artificial Sequence Synthetic primer
tcgtttggtggtggtagatgaaaaagg

SEQ ID NO: 389 - DNA, 11 bp, Artificial Sequence
  Description of Artificial Sequence Synthetic primer
tccaccctcaa

SEQ ID NO: 390 - DNA, 25 bp, Artificial Sequence
  Description of Artificial Sequence Synthetic primer
tctaaaacaccaggtcacccagaag

SEQ ID NO: 391 - DNA, 24 bp, Artificial Sequence
  Description of Artificial Sequence Synthetic primer
tctaaatggtcgtgcagttgcgtg

SEQ ID NO: 392 - DNA, 30 bp, Artificial Sequence
  Description of Artificial Sequence Synthetic primer
tctactgattttggtaatcttgcagcacag

SEQ ID NO: 393 - DNA, 24 bp, Artificial Sequence
  Description of Artificial Sequence Synthetic primer
tctagtaataataggaccctcagc

SEQ ID NO: 394 - DNA, 31 bp, Artificial Sequence
  Description of Artificial Sequence Synthetic primer
tctcaaggtgatattggtgtaggtaacttaa

SEQ ID NO: 395 - DNA, 24 bp, Artificial Sequence
  Description of Artificial Sequence Synthetic primer
tctcattacgttgcatcggaaaca

SEQ ID NO: 396 - DNA, 30 bp, Artificial Sequence
  Description of Artificial Sequence Synthetic primer
tctcgatgaacgaccaacaagtgattgatg

SEQ ID NO: 397 - DNA, 28 bp, Artificial Sequence
  Description of Artificial Sequence Synthetic primer
tctcgtggtgcacaagtaacggatatta

SEQ ID NO: 398 - DNA, 32 bp, Artificial Sequence
  Description of Artificial Sequence Synthetic primer
tctgaaatgaatagtgatagaactgtaggcac

SEQ ID NO: 399 - DNA, 32 bp, Artificial Sequence
  Description of Artificial Sequence Synthetic primer
tctgaacatgataatatctttgaaatcggctc

SEQ ID NO: 400 - DNA, 30 bp, Artificial Sequence
  Description of Artificial Sequence Synthetic primer
tctgaatgtctatatggaggtacaacacta

SEQ ID NO: 401 - DNA, 19 bp, Artificial Sequence
  Description of Artificial Sequence Synthetic primer
tctgacacctgcccggtgc

SEQ ID NO: 402 - DNA, 20 bp, Artificial Sequence
  Description of Artificial Sequence Synthetic primer
tctgcccgtgtcgttggtga

SEQ ID NO: 403 - DNA, 24 bp, Artificial Sequence
  Description of Artificial Sequence Synthetic primer
tctggaggcacaccaaataaaaca

SEQ ID NO: 404 - DNA, 21 bp, Artificial Sequence
  Description of Artificial Sequence Synthetic primer
tctggataacggtcgtcgcgg

SEQ ID NO: 405 - DNA, 25 bp, Artificial Sequence
  Description of Artificial Sequence Synthetic primer
tctggcaggtatgcgtggtctgatg

SEQ ID NO: 406 - DNA, 24 bp, Artificial Sequence
  Description of Artificial Sequence Synthetic primer
tctggctaaaactttggcaacggt

SEQ ID NO: 407 - DNA, 29 bp, Artificial Sequence
  Description of Artificial Sequence Synthetic primer
tctggtccaacaaaaggaacgattacagg

SEQ ID NO: 408 - DNA, 25 bp, Artificial Sequence
  Description of Artificial Sequence Synthetic primer
tctgtccctagtacgagaggaccgg

SEQ ID NO: 409 - DNA, 23 bp, Artificial Sequence
  Description of Artificial Sequence Synthetic primer
tctgttcttagtacgagaggacc

SEQ ID NO: 410 - DNA, 26 bp, Artificial Sequence
  Description of Artificial Sequence Synthetic primer
tcttatgccaagaggacagagtgagt

SEQ ID NO: 411 - DNA, 29 bp, Artificial Sequence
  Description of Artificial Sequence Synthetic primer
tcttatgccaagaggacagagtgagtact

SEQ ID NO: 412 - DNA, 31 bp, Artificial Sequence
  Description of Artificial Sequence Synthetic primer
tcttattccaacttcaaaccgaactatgacg

SEQ ID NO: 413 - DNA, 29 bp, Artificial Sequence
  Description of Artificial Sequence Synthetic primer
tcttctcatcctatggctattatgcttgc

SEQ ID NO: 414 - DNA, 33 bp, Artificial Sequence
  Description of Artificial Sequence Synthetic primer
tcttgatacttgtaatgtgggcgataaatatgt

SEQ ID NO: 415 - DNA, 32 bp, Artificial Sequence
  Description of Artificial Sequence Synthetic primer
tcttgcagcagtttatttgatgaacctaaagt

SEQ ID NO: 416 - DNA, 28 bp, Artificial Sequence
  Description of Artificial Sequence Synthetic primer
tcttgctctttcgtgagttcagtaaatg

SEQ ID NO: 417 - DNA, 31 bp, Artificial Sequence
  Description of Artificial Sequence Synthetic primer
tcttgtacttgtggctcacacggctgtttgg

SEQ ID NO: 418 - DNA, 27 bp, Artificial Sequence
  Description of Artificial Sequence Synthetic primer
tcttgtttatgctggtaaagcagatgg

SEQ ID NO: 419 - DNA, 28 bp, Artificial Sequence
  Description of Artificial Sequence Synthetic primer
tctttatggtggagatgactgaaaccga

SEQ ID NO: 420 - DNA, 28 bp, Artificial Sequence
  Description of Artificial Sequence Synthetic primer
tctttcttgaatgctggtgtacgtatcg

SEQ ID NO: 421 - DNA, 25 bp, Artificial Sequence
  Description of Artificial Sequence Synthetic primer
tctttgaaatcggctcaggaaaagg

SEQ ID NO: 422 - DNA, 26 bp, Artificial Sequence
  Description of Artificial Sequence Synthetic primer
tctttgccattgaagatgacttaagc

SEQ ID NO: 423 - DNA, 29 bp, Artificial Sequence
  Description of Artificial Sequence Synthetic primer
tcttttacaaaaggggaaaaagttgactt

SEQ ID NO: 424 - DNA, 34 bp, Artificial Sequence
  Description of Artificial Sequence Synthetic primer
tgaaaaatgtccaagaagcatagcaaaaaaagca

SEQ ID NO: 425 - DNA, 30 bp, Artificial Sequence
  Description of Artificial Sequence Synthetic primer
tgaaaagggtgaagtagcaaatggagatag

SEQ ID NO: 426 - DNA, 32 bp, Artificial Sequence
  Description of Artificial Sequence Synthetic primer
tgaaaagtatggatttgaacaactcgtgaata

SEQ ID NO: 427 - DNA, 27 bp, Artificial Sequence
  Description of Artificial Sequence Synthetic primer
tgaaatctcattacgttgcatcggaaa

SEQ ID NO: 428 - DNA, 30 bp, Artificial Sequence
  Description of Artificial Sequence Synthetic primer
tgaaattgctacaggccctttaggacaagg

SEQ ID NO: 429 - DNA, 25 bp, Artificial Sequence
  Description of Artificial Sequence Synthetic primer
tgaacgctggtggcatgcttaacac

SEQ ID NO: 430 - DNA, 30 bp, Artificial Sequence
  Description of Artificial Sequence Synthetic primer
tgaacgtggtcaaatcaaagttggtgaaga

SEQ ID NO: 431 - DNA, 31 bp, Artificial Sequence
  Description of Artificial Sequence Synthetic primer
tgaacgtggtcaaatcaaagttggtgaagaa

SEQ ID NO: 432 - DNA, 27 bp, Artificial Sequence
  Description of Artificial Sequence Synthetic primer
tgaagcttgttctttagcaggacttca

SEQ ID NO: 433 - DNA, 28 bp, Artificial Sequence
  Description of Artificial Sequence Synthetic primer
tgaaggtggacgtcacactccattcttc

SEQ ID NO: 434 - DNA, 26 bp, Artificial Sequence
  Description of Artificial Sequence Synthetic primer
tgaagtagaaatgactgaacgtccga

SEQ ID NO: 435 - DNA, 28 bp, Artificial Sequence
  Description of Artificial Sequence Synthetic primer
tgaagtagaaggtgcaaagcaagttaga

SEQ ID NO: 436 - DNA, 34 bp, Artificial Sequence
  Description of Artificial Sequence Synthetic primer
tgaagtgcgtgatgatatcgatgcacttgatgta

SEQ ID NO: 437 - DNA, 31 bp, Artificial Sequence
  Description of Artificial Sequence Synthetic primer
tgaatagtgatagaactgtaggcacaatcgt

SEQ ID NO: 438 - DNA, 31 bp, Artificial Sequence
  Description of Artificial Sequence Synthetic primer
tgaatgcttatttacctgcactcccacaact

SEQ ID NO: 439 - DNA, 31 bp, Artificial Sequence
  Description of Artificial Sequence Synthetic primer
tgaattagttcaatcatttgttgaacgacgt

SEQ ID NO: 440 - DNA, 26 bp, Artificial Sequence
  Description of Artificial Sequence Synthetic primer
tgaattgctgctatgaaaggtggctt

SEQ ID NO: 441 - DNA, 27 bp, Artificial Sequence
  Description of Artificial Sequence Synthetic primer
tgacagcgaagaaggttagacttgtcc

SEQ ID NO: 442 - DNA, 26 bp, Artificial Sequence
  Description of Artificial Sequence Synthetic primer
tgacatccggctcacgttattatggt

SEQ ID NO: 443 - DNA, 27 bp, Artificial Sequence
  Description of Artificial Sequence Synthetic primer
tgacatccggctcacgttattatggta

SEQ ID NO: 444 - DNA, 28 bp, Artificial Sequence
  Description of Artificial Sequence Synthetic primer
tgacatccggctcacgttattatggtac

SEQ ID NO: 445 - DNA, 29 bp, Artificial Sequence
  Description of Artificial Sequence Synthetic primer
tgacatgataataaccgattgaccgaaga

SEQ ID NO: 446 - DNA, 22 bp, Artificial Sequence
  Description of Artificial Sequence Synthetic primer
tgacatgcttgtccgttcaggc

SEQ ID NO: 447 - DNA, 31 bp, Artificial Sequence
  Description of Artificial Sequence Synthetic primer
tgacatggactccccctatataactcttgag

SEQ ID NO: 448 - DNA, 23 bp, Artificial Sequence
  Description of Artificial Sequence Synthetic primer
tgaccaggtgatggccatgttcg

SEQ ID NO: 449 - DNA, 31 bp, Artificial Sequence
  Description of Artificial Sequence Synthetic primer
tgacctacagtaagaggttctgtaatgaacc

SEQ ID NO: 450 - DNA, 23 bp, Artificial Sequence
  Description of Artificial Sequence Synthetic primer
tgacgatcttcgcggtgactagt

SEQ ID NO: 451 - DNA, 26 bp, Artificial Sequence
  Description of Artificial Sequence Synthetic primer
tgacggcctatacggtgttggtttct

SEQ ID NO: 452 - DNA, 24 bp, Artificial Sequence
  Description of Artificial Sequence Synthetic primer
tgacgtcatcggtaagtaccaccc

SEQ ID NO: 453 - DNA, 27 bp, Artificial Sequence
  Description of Artificial Sequence Synthetic primer
tgagatggatttaaacctgttcaccgc

SEQ ID NO: 454 - DNA, 30 bp, Artificial Sequence
  Description of Artificial Sequence Synthetic primer
tgagattgctgaacatttaatgctgattga

SEQ ID NO: 455 - DNA, 32 bp, Artificial Sequence
  Description of Artificial Sequence Synthetic primer
tgagcaatggggctttgaaagaatttttaaat

SEQ ID NO: 456 - DNA, 26 bp, Artificial Sequence
  Description of Artificial Sequence Synthetic primer
tgagctgcatcaactgtattggatag

SEQ ID NO: 457 - DNA, 28 bp, Artificial Sequence
  Description of Artificial Sequence Synthetic primer
tgagcttttagttgactttttcaacagc

SEQ ID NO: 458 - DNA, 20 bp, Artificial Sequence
  Description of Artificial Sequence Synthetic primer
tgaggaccgtgtcgcgctca

SEQ ID NO: 459 - DNA, 35 bp, Artificial Sequence
  Description of Artificial Sequence Synthetic primer
tgagggttttatgcttaaagttggttttattggtt

SEQ ID NO: 460 - DNA, 30 bp, Artificial Sequence
  Description of Artificial Sequence Synthetic primer
tgaggtggtggataactcaattgatgaagc

SEQ ID NO: 461 - DNA, 29 bp, Artificial Sequence
  Description of Artificial Sequence Synthetic primer
tgagtaacatccatatttctgccatacgt

SEQ ID NO: 462 - DNA, 22 bp, Artificial Sequence
  Description of Artificial Sequence Synthetic primer
tgagtaagttccacccgcacgg

SEQ ID NO: 463 - DNA, 30 bp, Artificial Sequence
  Description of Artificial Sequence Synthetic primer
tgagtcacttgaagttgatacaaatcctct

SEQ ID NO: 464 - DNA, 28 bp, Artificial Sequence
  Description of Artificial Sequence Synthetic primer
tgagtgatgaaggccttagggttgtaaa

SEQ ID NO: 465 - DNA, 27 bp, Artificial Sequence
  Description of Artificial Sequence Synthetic primer
tgagtgccaacatatcagtgctgaaga

SEQ ID NO: 466 - DNA, 30 bp, Artificial Sequence
  Description of Artificial Sequence Synthetic primer
tgagtttaacagttcaccatatgaaacagg

SEQ ID NO: 467 - DNA, 25 bp, Artificial Sequence
  Description of Artificial Sequence Synthetic primer
tgatacttcaacgcctgctgctttc

SEQ ID NO: 468 - DNA, 25 bp, Artificial Sequence
  Description of Artificial Sequence Synthetic primer
tgatcactggtgctgctcagatgga

SEQ ID NO: 469 - DNA, 30 bp, Artificial Sequence
  Description of Artificial Sequence Synthetic primer
tgatcatccgtggtataacgatttattagt

SEQ ID NO: 470 - DNA, 29 bp, Artificial Sequence
  Description of Artificial Sequence Synthetic primer
tgatcgttgagaagggatttgcgaaaaga

SEQ ID NO: 471 - DNA, 29 bp, Artificial Sequence
  Description of Artificial Sequence Synthetic primer
tgatctcagaatctaataattgggacgaa

SEQ ID NO: 472 - DNA, 30 bp, Artificial Sequence
  Description of Artificial Sequence Synthetic primer
tgatcttaaaaatttccgccaacttcattc

SEQ ID NO: 473 - DNA, 30 bp, Artificial Sequence
  Description of Artificial Sequence Synthetic primer
tgatgactttttagctaatggtcaggcagc

SEQ ID NO: 474 - DNA, 29 bp, Artificial Sequence
  Description of Artificial Sequence Synthetic primer
tgatggcaagtggatagggtataatacag

SEQ ID NO: 475 - DNA, 24 bp, Artificial Sequence
  Description of Artificial Sequence Synthetic primer
tgattaccatgagtggcaagcaag

SEQ ID NO: 476 - DNA, 31 bp, Artificial Sequence
  Description of Artificial Sequence Synthetic primer
tgattattgttatcctgttatgccatttgag

SEQ ID NO: 477 - DNA, 19 bp, Artificial Sequence
  Description of Artificial Sequence Synthetic primer
tgattccggtgcccgtggt

SEQ ID NO: 478 - DNA, 19 bp, Artificial Sequence
  Description of Artificial Sequence Synthetic primer
tgattctggtgcccgtggt

SEQ ID NO: 479 - DNA, 34 bp, Artificial Sequence
  Description of Artificial Sequence Synthetic primer
tgattttgctaaatttagagaaattgcggatgaa

SEQ ID NO: 480 - DNA, 24 bp, Artificial Sequence
  Description of Artificial Sequence Synthetic primer
tgcaaaatctgcaacgagctttgg

SEQ ID NO: 481 - DNA, 23 bp, Artificial Sequence
  Description of Artificial Sequence Synthetic primer
tgcaaaggaggtactcagaccat

SEQ ID NO: 482 - DNA, 25 bp, Artificial Sequence
  Description of Artificial Sequence Synthetic primer
tgcaagcaaacgcacaatcagaagc

SEQ ID NO: 483 - DNA, 20 bp, Artificial Sequence
  Description of Artificial Sequence Synthetic primer
tgcaagcgcgaccacatacg

SEQ ID NO: 484 - DNA, 23 bp, Artificial Sequence
  Description of Artificial Sequence Synthetic primer
tgcaagcttctggtgctagcatt

SEQ ID NO: 485 - DNA, 24 bp, Artificial Sequence
  Description of Artificial Sequence Synthetic primer
tgcaagtggtacttcaacatgggg

SEQ ID NO: 486 - DNA, 30 bp, Artificial Sequence
  Description of Artificial Sequence Synthetic primer
tgcaagttaagaaagctgttgcaggtttat

SEQ ID NO: 487 - DNA, 33 bp, Artificial Sequence
  Description of Artificial Sequence Synthetic primer
tgcaattgctttagttttaagtgcatgtaattc

SEQ ID NO: 488 - DNA, 31 bp, Artificial Sequence
  Description of Artificial Sequence Synthetic primer
tgcacaatcagaagctaagaaagcgcaagct

SEQ ID NO: 489 - DNA, 24 bp, Artificial Sequence
  Description of Artificial Sequence Synthetic primer
tgcacacgccgttcttcaacaact

SEQ ID NO: 490 - DNA, 22 bp, Artificial Sequence
  Description of Artificial Sequence Synthetic primer
tgcacatcgtgtccaacgtcac

SEQ ID NO: 491 - DNA, 32 bp, Artificial Sequence
  Description of Artificial Sequence Synthetic primer
tgcaccggctattaagaattactttgccaact

SEQ ID NO: 492 - DNA, 23 bp, Artificial Sequence
  Description of Artificial Sequence Synthetic primer
tgcacgatgcggaatggttcaca

SEQ ID NO: 493 - DNA, 28 bp, Artificial Sequence
  Description of Artificial Sequence Synthetic primer
tgcacgccgactatgttaagaacatgat

SEQ ID NO: 494 - DNA, 30 bp, Artificial Sequence
  Description of Artificial Sequence Synthetic primer
tgcacttatcgcaaatggaaaattaagcaa

SEQ ID NO: 495 - DNA, 21 bp, Artificial Sequence
  Description of Artificial Sequence Synthetic primer
tgcagggaacagctttaggca

SEQ ID NO: 496 - DNA, 22 bp, Artificial Sequence
  Description of Artificial Sequence Synthetic primer
tgcatacaaacagtcggagcct

SEQ ID NO: 497 - DNA, 23 bp, Artificial Sequence
  Description of Artificial Sequence Synthetic primer
tgcataccggtaagttggcaaca

SEQ ID NO: 498 - DNA, 27 bp, Artificial Sequence
  Description of Artificial Sequence Synthetic primer
tgcatattatatcgagccacagcatcg

SEQ ID NO: 499 - DNA, 33 bp, Artificial Sequence
  Description of Artificial Sequence Synthetic primer
tgcattatttggaactattgcaactgctaatgc

SEQ ID NO: 500 - DNA, 28 bp, Artificial Sequence
  Description of Artificial Sequence Synthetic primer
tgccaagaggacagagtgagtactttga

SEQ ID NO: 501 - DNA, 31 bp, Artificial Sequence
  Description of Artificial Sequence Synthetic primer
tgccggacaattacgattcatcgagtattaa

SEQ ID NO: 502 - DNA, 33 bp, Artificial Sequence
  Description of Artificial Sequence Synthetic primer
tgccgtaatgataggtgaagatatacaaagagt

SEQ ID NO: 503 - DNA, 23 bp, Artificial Sequence
  Description of Artificial Sequence Synthetic primer
tgccgtgttgaacgtggtcaaat

SEQ ID NO: 504 - DNA, 33 bp, Artificial Sequence
  Description of Artificial Sequence Synthetic primer
tgcctagaagatcttaaaaatttccgccaactt

SEQ ID NO: 505 - DNA, 33 bp, Artificial Sequence
  Description of Artificial Sequence Synthetic primer
tgcctatctttttgctgatatagcacatattgc

SEQ ID NO: 506 - DNA, 27 bp, Artificial Sequence
  Description of Artificial Sequence Synthetic primer
tgcctcgaagctgaatataaccaagtt

SEQ ID NO: 507 - DNA, 22 bp, Artificial Sequence
  Description of Artificial Sequence Synthetic primer
tgcctgtagggaatcctgctga

SEQ ID NO: 508 - DNA, 25 bp, Artificial Sequence
  Description of Artificial Sequence Synthetic primer
tgcctgttcttagtacgagaggacc

SEQ ID NO: 509 - DNA, 23 bp, Artificial Sequence
  Description of Artificial Sequence Synthetic primer
tgcgcagctcttggtatcgagtt

SEQ ID NO: 510 - DNA, 20 bp, Artificial Sequence
  Description of Artificial Sequence Synthetic primer
tgcgcggaagatgtaacggg

SEQ ID NO: 511 - DNA, 30 bp, Artificial Sequence
  Description of Artificial Sequence Synthetic primer
tgcggatcgtttggtggttgtagatgaaaa

SEQ ID NO: 512 - DNA, 20 bp, Artificial Sequence
  Description of Artificial Sequence Synthetic primer
tgcgggtagggagcttgagc

SEQ ID NO: 513 - DNA, 31 bp, Artificial Sequence
  Description of Artificial Sequence Synthetic primer
tgcgtacaatacgctttatgaaattttaaca

SEQ ID NO: 514 - DNA, 27 bp, Artificial Sequence
  Description of Artificial Sequence Synthetic primer
tgcgtataaaaaacacagatggcagca

SEQ ID NO: 515 - DNA, 21 bp, Artificial Sequence
  Description of Artificial Sequence Synthetic primer
tgcgtttaccgcaatgcgtgc

SEQ ID NO: 516 - DNA, 29 bp, Artificial Sequence
  Description of Artificial Sequence Synthetic primer
tgctacggtaggatctccttatcctattg

SEQ ID NO: 517 - DNA, 30 bp, Artificial Sequence
  Description of Artificial Sequence Synthetic primer
tgctagtcaatctatcattccggttgatac

SEQ ID NO: 518 - DNA, 27 bp, Artificial Sequence
  Description of Artificial Sequence Synthetic primer
tgctagttatggtacagagtttgcgac

SEQ ID NO: 519 - DNA, 26 bp, Artificial Sequence
  Description of Artificial Sequence Synthetic primer
tgctatggtgttaccttccctatgca

SEQ ID NO: 520 - DNA, 27 bp, Artificial Sequence
  Description of Artificial Sequence Synthetic primer
tgctcaacccgatcctaaattagacga

SEQ ID NO: 521 - DNA, 27 bp, Artificial Sequence
  Description of Artificial Sequence Synthetic primer
tgctcaatctaaacctaaagtcgaaga

SEQ ID NO: 522 - DNA, 33 bp, Artificial Sequence
  Description of Artificial Sequence Synthetic primer
tgctcgagtgattgactttgctaaatttagaga

SEQ ID NO: 523 - DNA, 24 bp, Artificial Sequence
  Description of Artificial Sequence Synthetic primer
tgctcgtaagggtctggcggatac

SEQ ID NO: 524 - DNA, 29 bp, Artificial Sequence
  Description of Artificial Sequence Synthetic primer
tgctcgtggtgcacaagtaacggatatta

SEQ ID NO: 525 - DNA, 26 bp, Artificial Sequence
  Description of Artificial Sequence Synthetic primer
tgctgaggcctggaccgattatttac

SEQ ID NO: 526 - DNA, 27 bp, Artificial Sequence
  Description of Artificial Sequence Synthetic primer
tgctggtaacagagccttataggcgca

SEQ ID NO: 527 - DNA, 19 bp, Artificial Sequence
  Description of Artificial Sequence Synthetic primer
tgctggtacgggtcgagga

SEQ ID NO: 528 - DNA, 31 bp, Artificial Sequence
  Description of Artificial Sequence Synthetic primer
tgctggtgaaaataacccagatgtcgtcttc

SEQ ID NO: 529 - DNA, 32 bp, Artificial Sequence
  Description of Artificial Sequence Synthetic primer
tgctgtagcttatcgcgaaatgtctttgattt

SEQ ID NO: 530 - DNA, 28 bp, Artificial Sequence
  Description of Artificial Sequence Synthetic primer
tgcttatttacctgcactcccacaactg

SEQ ID NO: 531 - DNA, 26 bp, Artificial Sequence
  Description of Artificial Sequence Synthetic primer
tgcttcaggaatcaatgatggagcag

SEQ ID NO: 532 - DNA, 27 bp, Artificial Sequence
  Description of Artificial Sequence Synthetic primer
tgcttcggatccagcagcacttcaata

SEQ ID NO: 533 - DNA, 19 bp, Artificial Sequence
  Description of Artificial Sequence Synthetic primer
tgcttctggtgctagcatt

SEQ ID NO: 534 - DNA, 32 bp, Artificial Sequence
  Description of Artificial Sequence Synthetic primer
tgctttcctatggcttatccaaatttagatcg

SEQ ID NO: 535 - DNA, 29 bp, Artificial Sequence
  Description of Artificial Sequence Synthetic primer
tgcttttgatggtgatgcagatcgtttgg

SEQ ID NO: 536 - DNA, 24 bp, Artificial Sequence
  Description of Artificial Sequence Synthetic primer
tggaaagccatgcgtctgacatct

SEQ ID NO: 537 - DNA, 23 bp, Artificial Sequence
  Description of Artificial Sequence Synthetic primer
tggaaaggtgttgcagctactca

SEQ ID NO: 538 - DNA, 31 bp, Artificial Sequence
  Description of Artificial Sequence Synthetic primer
tggaaatggcagctagaatagtagctaaaat

SEQ ID NO: 539 - DNA, 30 bp, Artificial Sequence
  Description of Artificial Sequence Synthetic primer
tggaacaaaatagtctctcggattttgact

SEQ ID NO: 540 - DNA, 33 bp, Artificial Sequence
  Description of Artificial Sequence Synthetic primer
tggaacaggaattaattctcatcctgattatcc

SEQ ID NO: 541 - DNA, 30 bp, Artificial Sequence
  Description of Artificial Sequence Synthetic primer
tggaacgttatcaggtgccccaaaaattcg

SEQ ID NO: 542 - DNA, 23 bp, Artificial Sequence
  Description of Artificial Sequence Synthetic primer
tggaactattgcaactgctaatg

SEQ ID NO: 543 - DNA, 30 bp, Artificial Sequence
  Description of Artificial Sequence Synthetic primer
tggaacttgaagctctcgctcttaaagatg

SEQ ID NO: 544 - DNA, 19 bp, Artificial Sequence
  Description of Artificial Sequence Synthetic primer
tggaagatctgggtcaggc

SEQ ID NO: 545 - DNA, 22 bp, Artificial Sequence
  Description of Artificial Sequence Synthetic primer
tggaagtcatcaagcgcctggc

SEQ ID NO: 546 - DNA, 30 bp, Artificial Sequence
  Description of Artificial Sequence Synthetic primer
tggaataacaaaacatgaaggaaaccactt

SEQ ID NO: 547 - DNA, 32 bp, Artificial Sequence
  Description of Artificial Sequence Synthetic primer
tggaatgatgataaagatttcgcagatagcta

SEQ ID NO: 548 - DNA, 29 bp, Artificial Sequence
  Description of Artificial Sequence Synthetic primer
tggacaatagacaatcacttggatttaca

SEQ ID NO: 549 - DNA, 28 bp, Artificial Sequence
  Description of Artificial Sequence Synthetic primer
tggacacatatcgtgagcaatgaactga

SEQ ID NO: 550 - DNA, 23 bp, Artificial Sequence
  Description of Artificial Sequence Synthetic primer
tggacggcatcacgattctctac

SEQ ID NO: 551 - DNA, 19 bp, Artificial Sequence
  Description of Artificial Sequence Synthetic primer
tggactcctcggtggtcgc

SEQ ID NO: 552 - DNA, 19 bp, Artificial Sequence
  Description of Artificial Sequence Synthetic primer
tggagcacggcttctgatc

SEQ ID NO: 553 - DNA, 30 bp, Artificial Sequence
  Description of Artificial Sequence Synthetic primer
tggagcttgaagctatcgctcttaaagatg

SEQ ID NO: 554 - DNA, 22 bp, Artificial Sequence
  Description of Artificial Sequence Synthetic primer
tggaggtgtcactccacacgaa

SEQ ID NO: 555 - DNA, 25 bp, Artificial Sequence
  Description of Artificial Sequence Synthetic primer
tggaggttgttgtatgtatggtggt

SEQ ID NO: 556 - DNA, 26 bp, Artificial Sequence
  Description of Artificial Sequence Synthetic primer
tggatattcaccgaacactagggttg

SEQ ID NO: 557 - DNA, 27 bp, Artificial Sequence
  Description of Artificial Sequence Synthetic primer
tggatggcatggtgaaatggatatgtc

SEQ ID NO: 558 - DNA, 25 bp, Artificial Sequence
  Description of Artificial Sequence Synthetic primer
tggatggggattagcggttacaatg

SEQ ID NO: 559 - DNA, 26 bp, Artificial Sequence
  Description of Artificial Sequence Synthetic primer
tggatgttaagggtgattttcccgaa

SEQ ID NO: 560 - DNA, 23 bp, Artificial Sequence
  Description of Artificial Sequence Synthetic primer
tggattagagaccctggtagtcc

SEQ ID NO: 561 - DNA, 19 bp, Artificial Sequence
  Description of Artificial Sequence Synthetic primer
tggcacggccatctccgtg

SEQ ID NO: 562 - DNA, 34 bp, Artificial Sequence
  Description of Artificial Sequence Synthetic primer
tggcactcttgcctttaatattagtaaactatca

SEQ ID NO: 563 - DNA, 31 bp, Artificial Sequence
  Description of Artificial Sequence Synthetic primer
tggcagctagaatagtagctaaaatccctac

SEQ ID NO: 564 - DNA, 28 bp, Artificial Sequence
  Description of Artificial Sequence Synthetic primer
tggcagttttacaaggtgctgtttcatc

SEQ ID NO: 565 - DNA, 31 bp, Artificial Sequence
  Description of Artificial Sequence Synthetic primer
tggcatttcttatgaagcttgttctttagca

SEQ ID NO: 566 - DNA, 24 bp, Artificial Sequence
  Description of Artificial Sequence Synthetic primer
tggccagcgcttcggtgaaatgga

SEQ ID NO: 567 - DNA, 21 bp, Artificial Sequence
  Description of Artificial Sequence Synthetic primer
tggcccgaaagaagctgagcg

SEQ ID NO: 568 - DNA, 32 bp, Artificial Sequence
  Description of Artificial Sequence Synthetic primer
tggcctaatgggcttaatatcaatgaaaattg

SEQ ID NO: 569 - DNA, 22 bp, Artificial Sequence
  Description of Artificial Sequence Synthetic primer
tggcgaacctggtgaacgaagc

SEQ ID NO: 570 - DNA, 26 bp, Artificial Sequence
  Description of Artificial Sequence Synthetic primer
tggcgagtggatagggtataatacag

SEQ ID NO: 571 - DNA, 27 bp, Artificial Sequence
  Description of Artificial Sequence Synthetic primer
tggcgtagtagagctatttacagacac

SEQ ID NO: 572 - DNA, 26 bp, Artificial Sequence
  Description of Artificial Sequence Synthetic primer
tggcaagtggatagggtataatacag

SEQ ID NO: 573 - DNA, 25 bp, Artificial Sequence
  Description of Artificial Sequence Synthetic primer
tggctccttggtatgactctgcttc

SEQ ID NO: 574 - DNA, 25 bp, Artificial Sequence
  Description of Artificial Sequence Synthetic primer
tggctgacatcctacatgactgtga

SEQ ID NO: 575 - DNA, 31 bp, Artificial Sequence
  Description of Artificial Sequence Synthetic primer
tggcttatccaaatttagatcgtggttttac

SEQ ID NO: 576 - DNA, 30 bp, Artificial Sequence
  Description of Artificial Sequence Synthetic primer
tgggacttgaagctatcgctcttaaagatg

SEQ ID NO: 577 - DNA, 30 bp, Artificial Sequence
  Description of Artificial Sequence Synthetic primer
tgggatgaaaaagcgttcttttatccatga

SEQ ID NO: 578 - DNA, 34 bp, Artificial Sequence
  Description of Artificial Sequence Synthetic primer
tgggattattgttatcctgttatgccatttgaga

SEQ ID NO: 579 - DNA, 32 bp, Artificial Sequence
  Description of Artificial Sequence Synthetic primer
tgggattttaaaaaacattggtaacatcgcag

SEQ ID NO: 580 - DNA, 30 bp, Artificial Sequence
  Description of Artificial Sequence Synthetic primer
tgggcaacagcagcggattgcgattgcgcg

SEQ ID NO: 581 - DNA, 24 bp, Artificial Sequence
  Description of Artificial Sequence Synthetic primer
tgggcagcgtttcggcgaaatgga

SEQ ID NO: 582 - DNA, 33 bp, Artificial Sequence
  Description of Artificial Sequence Synthetic primer
tgggcctaatgggcttaatatcaatgaaaattg

SEQ ID NO: 583 - DNA, 28 bp, Artificial Sequence
  Description of Artificial Sequence Synthetic primer
tgggcgatgctgcgaaatggttaaaaga

SEQ ID NO: 584 - DNA, 27 bp, Artificial Sequence
  Description of Artificial Sequence Synthetic primer
tgggcgtgagcaatgaactgattatac

SEQ ID NO: 585 - DNA, 18 bp, Artificial Sequence
  Description of Artificial Sequence Synthetic primer
tgggcgtggaacgtccac

SEQ ID NO: 586 - DNA, 24 bp, Artificial Sequence
  Description of Artificial Sequence Synthetic primer
tgggctctttctcgcttaaacacc

SEQ ID NO: 587 - DNA, 25 bp, Artificial Sequence
  Description of Artificial Sequence Synthetic primer
tgggctctttctcgcttaaacacct

SEQ ID NO: 588 - DNA, 31 bp, Artificial Sequence
  Description of Artificial Sequence Synthetic primer
tggggattcagccatcaaagcagctattgac

SEQ ID NO: 589 - DNA, 28 bp, Artificial Sequence
  Description of Artificial Sequence Synthetic primer
tggggattgatatcaccgataagaagaa

SEQ ID NO: 590 - DNA, 33 bp, Artificial Sequence
  Description of Artificial Sequence Synthetic primer
tggggctttaaatattccaattgaagattttca

SEQ ID NO: 591 - DNA, 33 bp, Artificial Sequence
  Description of Artificial Sequence Synthetic primer
tggggctttgctttatagttttttacatttaag

SEQ ID NO: 592 - DNA, 28 bp, Artificial Sequence
  Description of Artificial Sequence Synthetic primer
tgggngatgctgcnaaatggttaaaaga

SEQ ID NO: 593 - DNA, 35 bp, Artificial Sequence
  Description of Artificial Sequence Synthetic primer
tgggtcgtggttttacagaaaatttcttatatatg

SEQ ID NO: 594 - DNA, 28 bp, Artificial Sequence
  Description of Artificial Sequence Synthetic primer
tgggtgacattcatcaatttcatcgttc

SEQ ID NO: 595 - DNA, 32 bp, Artificial Sequence
  Description of Artificial Sequence Synthetic primer
tgggtttacacatatcgtgagcaatgaactga

SEQ ID NO: 596 - DNA, 25 bp, Artificial Sequence
  Description of Artificial Sequence Synthetic primer
tggtaaataccacccacatggtgac

SEQ ID NO: 597 - DNA, 24 bp, Artificial Sequence
  Description of Artificial Sequence Synthetic primer
tggtaacagagccttataggcgca

SEQ ID NO: 598 - DNA, 28 bp, Artificial Sequence
  Description of Artificial Sequence Synthetic primer
tggtaacagagccttataggcgcatatg

SEQ ID NO: 599 - DNA, 30 bp, Artificial Sequence
  Description of Artificial Sequence Synthetic primer
tggtaagagcgcaccggtaagttggtaaca

SEQ ID NO: 600 - DNA, 18 bp, Artificial Sequence
  Description of Artificial Sequence Synthetic primer
tggtacagagtttgcgac

SEQ ID NO: 601 - DNA, 26 bp, Artificial Sequence
  Description of Artificial Sequence Synthetic primer
tggtacatgtgccttcattgatgctg

SEQ ID NO: 602 - DNA, 18 bp, Artificial Sequence
  Description of Artificial Sequence Synthetic primer
tggtacagagtttgcgac

SEQ ID NO: 603 - DNA, 24 bp, Artificial Sequence
  Description of Artificial Sequence Synthetic primer
tggtactcacttagcgggtttccg

SEQ ID NO: 604 - DNA, 24 bp, Artificial Sequence
  Description of Artificial Sequence Synthetic primer
tggtatgatatgatgcctgcacca

SEQ ID NO: 605 - DNA, 21 bp, Artificial Sequence
  Description of Artificial Sequence Synthetic primer
tggtatgcgtggtctgatggc

SEQ ID NO: 606 - DNA, 31 bp, Artificial Sequence
  Description of Artificial Sequence Synthetic primer
tggtattctattttgctgataatgacctcgc

SEQ ID NO: 607 - DNA, 25 bp, Artificial Sequence
  Description of Artificial Sequence Synthetic primer
tggtcaaatcaaagttggtgaagaa

SEQ ID NO: 608 - DNA, 29 bp, Artificial Sequence
  Description of Artificial Sequence Synthetic primer
tggtcttatgccaagaggacagagtgagt

SEQ ID NO: 609 - DNA, 24 bp, Artificial Sequence
  Description of Artificial Sequence Synthetic primer
tggtgactcggcatgttatgaagc

SEQ ID NO: 610 - DNA, 30 bp, Artificial Sequence
  Description of Artificial Sequence Synthetic primer
tggtgacttcataatggatgaagttgaagt

SEQ ID NO: 611 - DNA, 27 bp, Artificial Sequence
  Description of Artificial Sequence Synthetic primer
tggtgcgagtgcttatgctcgtattat

SEQ ID NO: 612 - DNA, 13 bp, Artificial Sequence
  Description of Artificial Sequence Synthetic primer
tggtgctagcatt

SEQ ID NO: 613 - DNA, 24 bp, Artificial Sequence
  Description of Artificial Sequence Synthetic primer
tggtgctttctggcgcttaaacga

SEQ ID NO: 614 - DNA, 27 bp, Artificial Sequence
  Description of Artificial Sequence Synthetic primer
tggtggacatttaacacatggtgcaaa

SEQ ID NO: 615 - DNA, 26 bp, Artificial Sequence
  Description of Artificial Sequence Synthetic primer
tggtggtgaaatagataggactgctt

SEQ ID NO: 616 - DNA, 13 bp, Artificial Sequence
  Description of Artificial Sequence Synthetic primer
tggtgctagcatt

SEQ ID NO: 617 - DNA, 26 bp, Artificial Sequence
  Description of Artificial Sequence Synthetic primer
tggttatcgctcaggcgaactccaac

SEQ ID NO: 618 - DNA, 33 bp, Artificial Sequence
  Description of Artificial Sequence Synthetic primer
tggttatgtaccaaatactttgtctgaagatgg

SEQ ID NO: 619 - DNA, 31 bp, Artificial Sequence
  Description of Artificial Sequence Synthetic primer
tggtttagataattccttaggatctatgcgt

SEQ ID NO: 620 - DNA, 31 bp, Artificial Sequence
  Description of Artificial Sequence Synthetic primer
tgnccnacngtnngnggttctgtaatgaacc

SEQ ID NO: 621 - DNA, 24 bp, Artificial Sequence
  Description of Artificial Sequence Synthetic primer
tgtaactatcacccgcacggtgat

SEQ ID NO: 622 - DNA, 30 bp, Artificial Sequence
  Description of Artificial Sequence Synthetic primer
tgtaagctctacaacccacaaaaccttacg

SEQ ID NO: 623 - DNA, 30 bp, Artificial Sequence
  Description of Artificial Sequence Synthetic primer
tgtaatgaaccctaatgaccatccacacgg

SEQ ID NO: 624 - DNA, 31 bp, Artificial Sequence
  Description of Artificial Sequence Synthetic primer
tgtacccgctgaattaacgaatttatacgac

SEQ ID NO: 625 - DNA, 24 bp, Artificial Sequence
  Description of Artificial Sequence Synthetic primer
tgtactcggtaagtatcacccgca

SEQ ID NO: 626 - DNA, 21 bp, Artificial Sequence
  Description of Artificial Sequence Synthetic primer
tgtactgctatccaccctcaa

SEQ ID NO: 627 - DNA, 24 bp, Artificial Sequence
  Description of Artificial Sequence Synthetic primer
tgtagccgctaagcactaccatcc

SEQ ID NO: 628 - DNA, 30 bp, Artificial Sequence
  Description of Artificial Sequence Synthetic primer
tgtagcttatcgcgaaatgtctttgatttt

SEQ ID NO: 629 - DNA, 33 bp, Artificial Sequence
  Description of Artificial Sequence Synthetic primer
tgtatggtggtgtaacgttacatgataataatc

SEQ ID NO: 630 - DNA, 27 bp, Artificial Sequence
  Description of Artificial Sequence Synthetic primer
tgtattaggggcatacagtcctcatcc

SEQ ID NO: 631 - DNA, 24 bp, Artificial Sequence
  Description of Artificial Sequence Synthetic primer
tgtcaaagtggcacgtttactggc

SEQ ID NO: 632 - DNA, 24 bp, Artificial Sequence
  Description of Artificial Sequence Synthetic primer
tgtcatgggtaaatatcaccctca

SEQ ID NO: 633 - DNA, 28 bp, Artificial Sequence
  Description of Artificial Sequence Synthetic primer
tgtccaagaagcatagcaaaaaaagcaa

SEQ ID NO: 634 - DNA, 23 bp, Artificial Sequence
  Description of Artificial Sequence Synthetic primer
tgtcgatgcaacgcgaagaacct

SEQ ID NO: 635 - DNA, 25 bp, Artificial Sequence
  Description of Artificial Sequence Synthetic primer
tgtcggtacacgatattcttcacga

SEQ ID NO: 636 - DNA, 27 bp, Artificial Sequence
  Description of Artificial Sequence Synthetic primer
tgtgaataaatcacgattgattgagca

SEQ ID NO: 637 - DNA, 26 bp, Artificial Sequence
  Description of Artificial Sequence Synthetic primer
tgtggagtaacactgcatgaaaacaa

SEQ ID NO: 638 - DNA, 27 bp, Artificial Sequence
  Description of Artificial Sequence Synthetic primer
tgtggtcaaatcaaagttggtgaagaa

SEQ ID NO: 639 - DNA, 25 bp, Artificial Sequence
  Description of Artificial Sequence Synthetic primer
tgttcaagagctagatcttcaggca

SEQ ID NO: 640 - DNA, 26 bp, Artificial Sequence
  Description of Artificial Sequence Synthetic primer
tgttcaagagctagatcttcaggcaa

SEQ ID NO: 641 - DNA, 28 bp, Artificial Sequence
  Description of Artificial Sequence Synthetic primer
tgttcgctgtttcacaaacaacattcca

SEQ ID NO: 642 - DNA, 32 bp, Artificial Sequence
  Description of Artificial Sequence Synthetic primer
tgttctttagcaggacttcacaaacttgataa

SEQ ID NO: 643 - DNA, 29 bp, Artificial Sequence
  Description of Artificial Sequence Synthetic primer
tgttgaacgtggtcaaatcaaagttggtg

SEQ ID NO: 644 - DNA, 31 bp, Artificial Sequence
  Description of Artificial Sequence Synthetic primer
tgttgggagtattccttaccatttaagcaca

SEQ ID NO: 645 - DNA, 23 bp, Artificial Sequence
  Description of Artificial Sequence Synthetic primer
tgttggtgctttctggcgcttaa

SEQ ID NO: 646 - DNA, 38 bp, Artificial Sequence
  Description of Artificial Sequence Synthetic primer
tccttgntgnccnacngtnngnggttctgtaatgaacc

SEQ ID NO: 647 - DNA, 29 bp, Artificial Sequence
  Description of Artificial Sequence Synthetic primer
ttaaagttggttttattggttggcgcgga

SEQ ID NO: 648 - DNA, 30 bp, Artificial Sequence
  Description of Artificial Sequence Synthetic primer
ttaacatgaaggaaaccactttgataatgg

SEQ ID NO: 649 - DNA, 26 bp, Artificial Sequence
  Description of Artificial Sequence Synthetic primer
ttaacggttatcatggcccagatggg

SEQ ID NO: 650 - DNA, 22 bp, Artificial Sequence
  Description of Artificial Sequence Synthetic primer
ttaagtcccgcaacgagcgcaa

SEQ ID NO: 651 - DNA, 22 bp, Artificial Sequence
  Description of Artificial Sequence Synthetic primer
ttaagtcccgcaacgatcgcaa

SEQ ID NO: 652 - DNA, 28 bp, Artificial Sequence
  Description of Artificial Sequence Synthetic primer
ttaatttgccaaaaatgcaaccaggtag

SEQ ID NO: 653 - DNA, 27 bp, Artificial Sequence
  Description of Artificial Sequence Synthetic primer
ttacacatatcgtgagcaatgaactga

SEQ ID NO: 654 - DNA, 31 bp, Artificial Sequence
  Description of Artificial Sequence Synthetic primer
ttacaggaagtttaggtggtaatctaaaagg

SEQ ID NO: 655 - DNA, 30 bp, Artificial Sequence
  Description of Artificial Sequence Synthetic primer
ttactccattattgcttggttacactttcc

SEQ ID NO: 656 - DNA, 35 bp, Artificial Sequence
  Description of Artificial Sequence Synthetic primer
ttataacttactgcaatctattcagttgcttggtg

SEQ ID NO: 657 - DNA, 26 bp, Artificial Sequence
  Description of Artificial Sequence Synthetic primer
ttataccggaaacttcccgaaaggag

SEQ ID NO: 658 - DNA, 32 bp, Artificial Sequence
  Description of Artificial Sequence Synthetic primer
ttatcagctagaccttttaggtaaagctaagc

SEQ ID NO: 659 - DNA, 23 bp, Artificial Sequence
  Description of Artificial Sequence Synthetic primer
ttatcgctcaggcgaactccaac

SEQ ID NO: 660 - DNA, 28 bp, Artificial Sequence
  Description of Artificial Sequence Synthetic primer
ttatcgtttgtggagctagtgcttatgc

SEQ ID NO: 661 - DNA, 30 bp, Artificial Sequence
  Description of Artificial Sequence Synthetic primer
ttatgaagcgtgttctttagcaggacttca

SEQ ID NO: 662 - DNA, 25 bp, Artificial Sequence
  Description of Artificial Sequence Synthetic primer
ttatggatggcaacgtgaaacgcgt

SEQ ID NO: 663 - DNA, 21 bp, Artificial Sequence
  Description of Artificial Sequence Synthetic primer
ttattgttatcctgttatgcc

SEQ ID NO: 664 - DNA, 25 bp, Artificial Sequence
  Description of Artificial Sequence Synthetic primer
ttatttacctgcactcccacaactg

SEQ ID NO: 665 - DNA, 33 bp, Artificial Sequence
  Description of Artificial Sequence Synthetic primer
ttcaaaaactccaggccatcctgaaatttcaac

SEQ ID NO: 666 - DNA, 28 bp, Artificial Sequence
  Description of Artificial Sequence Synthetic primer
ttcaacaggtaccaatgatttgatctca

SEQ ID NO: 667 - DNA, 28 bp, Artificial Sequence
  Description of Artificial Sequence Synthetic primer
ttcccaccgatatcatggcttaccacgg

SEQ ID NO: 668 - DNA, 25 bp, Artificial Sequence
  Description of Artificial Sequence Synthetic primer
ttccgtaagtcggctaaaacagtcg

SEQ ID NO: 669 - DNA, 24 bp, Artificial Sequence
  Description of Artificial Sequence Synthetic primer
ttcctccttttgaaagcgacggtt

SEQ ID NO: 670 - DNA, 17 bp, Artificial Sequence
  Description of Artificial Sequence Synthetic primer
ttcctcggccgcctggc

SEQ ID NO: 671 - DNA, 29 bp, Artificial Sequence
  Description of Artificial Sequence Synthetic primer
ttcctgaccgacccattattccctttatc

SEQ ID NO: 672 - DNA, 22 bp, Artificial Sequence
  Description of Artificial Sequence Synthetic primer
ttcgatgcaacgcgaagaacct

SEQ ID NO: 673 - DNA, 27 bp, Artificial Sequence
  Description of Artificial Sequence Synthetic primer
ttcgccaatcaaaactaagggaatggc

SEQ ID NO: 674 - DNA, 20 bp, Artificial Sequence
  Description of Artificial Sequence Synthetic primer
ttcggcggtcagcgcttcgg

SEQ ID NO: 675 - DNA, 26 bp, Artificial Sequence
  Description of Artificial Sequence Synthetic primer
ttctaaaacaccaggtcacccagaag

SEQ ID NO: 676 - DNA, 27 bp, Artificial Sequence
  Description of Artificial Sequence Synthetic primer
ttctatctcgttggtttattcggagtt

SEQ ID NO: 677 - DNA, 30 bp, Artificial Sequence
  Description of Artificial Sequence Synthetic primer
ttctgaatgtctatatggaggtacaacact

SEQ ID NO: 678 - DNA, 21 bp, Artificial Sequence
  Description of Artificial Sequence Synthetic primer
ttgactgcccaggtcacgctg

SEQ ID NO: 679 - DNA, 22 bp, Artificial Sequence
  Description of Artificial Sequence Synthetic primer
ttgactgcggcacaacacggat

SEQ ID NO: 680 - DNA, 33 bp, Artificial Sequence
  Description of Artificial Sequence Synthetic primer
ttgagaagacatccggctcacgttattatggta

SEQ ID NO: 681 - DNA, 31 bp, Artificial Sequence
  Description of Artificial Sequence Synthetic primer
ttgagggtatgcaccgtctttttgattcttt

SEQ ID NO: 682 - DNA, 24 bp, Artificial Sequence
  Description of Artificial Sequence Synthetic primer
ttgcaactgctgatttagctcaga

SEQ ID NO: 683 - DNA, 22 bp, Artificial Sequence
  Description of Artificial Sequence Synthetic primer
ttgcacaagcaaggcgctattt

SEQ ID NO: 684 - DNA, 28 bp, Artificial Sequence
  Description of Artificial Sequence Synthetic primer
ttgccaatgatattcgttggttagcaag

SEQ ID NO: 685 - DNA, 31 bp, Artificial Sequence
  Description of Artificial Sequence Synthetic primer
ttgcccgcggtgcggaagtaaccgatattac

SEQ ID NO: 686 - DNA, 23 bp, Artificial Sequence
  Description of Artificial Sequence Synthetic primer
ttgcgaatagaacgatggctcgt

SEQ ID NO: 687 - DNA, 30 bp, Artificial Sequence
  Description of Artificial Sequence Synthetic primer
ttgctcgtggtgcacaagtaacggatatta

SEQ ID NO: 688 - DNA, 31 bp, Artificial Sequence
  Description of Artificial Sequence Synthetic primer
ttgctcgtggtgcacaagtaacggatattac

SEQ ID NO: 689 - DNA, 31 bp, Artificial Sequence
  Description of Artificial Sequence Synthetic primer
ttgctcgtggtgcanaagtaacggatatnac

SEQ ID NO: 690 - DNA, 29 bp, Artificial Sequence
  Description of Artificial Sequence Synthetic primer
ttgcttaaagttggttttattggttggcg

SEQ ID NO: 691 - DNA, 32 bp, Artificial Sequence
  Description of Artificial Sequence Synthetic primer
ttggtcctttttatacgaaagaagaagttgaa

SEQ ID NO: 692 - DNA, 25 bp, Artificial Sequence
  Description of Artificial Sequence Synthetic primer
ttgtaaatgccggtgcttcagatcc

SEQ ID NO: 693 - DNA, 22 bp, Artificial Sequence
  Description of Artificial Sequence Synthetic primer
ttgtacacaccgcccgtcatac

SEQ ID NO: 694 - DNA, 31 bp, Artificial Sequence
  Description of Artificial Sequence Synthetic primer
ttgtagcacagcaaggcaaatttcctgaaac

SEQ ID NO: 695 - DNA, 30 bp, Artificial Sequence
  Description of Artificial Sequence Synthetic primer
ttgtatgtatggtggtgtaacgttacatga

SEQ ID NO: 696 - DNA, 27 bp, Artificial Sequence
  Description of Artificial Sequence Synthetic primer
ttgtatgtatggtggtgtaactgagca

SEQ ID NO: 697 - DNA, 23 bp, Artificial Sequence
  Description of Artificial Sequence Synthetic primer
tttaagtcccgcaacgagcgcaa

SEQ ID NO: 698 - DNA, 28 bp, Artificial Sequence
  Description of Artificial Sequence Synthetic primer
tttacacatatcgtgagcaatgaactga

SEQ ID NO: 699 - DNA, 30 bp, Artificial Sequence
  Description of Artificial Sequence Synthetic primer
tttacactacttttattcattgccctaacg

SEQ ID NO: 700 - DNA, 19 bp, Artificial Sequence
  Description of Artificial Sequence Synthetic primer
tttacagctttatgcaccg

SEQ ID NO: 701 - DNA, 29 bp, Artificial Sequence
  Description of Artificial Sequence Synthetic primer
tttcacacagcgtgtttatagttctacca

SEQ ID NO: 702 - DNA, 30 bp, Artificial Sequence
  Description of Artificial Sequence Synthetic primer
tttcacatgtaattttgatattcgcactga

SEQ ID NO: 703 - DNA, 28 bp, Artificial Sequence
  Description of Artificial Sequence Synthetic primer
tttcatcttatcgaggacccgaaatcga

SEQ ID NO: 704 - DNA, 25 bp, Artificial Sequence
  Description of Artificial Sequence Synthetic primer
tttcctccttttgaaagcgacggtt

SEQ ID NO: 705 - DNA, 22 bp, Artificial Sequence
  Description of Artificial Sequence Synthetic primer
tttcgaagggcctttcgacctg

SEQ ID NO: 706 - DNA, 23 bp, Artificial Sequence
  Description of Artificial Sequence Synthetic primer
tttcgatgcaacgcgaagaacct

SEQ ID NO: 707 - DNA, 27 bp, Artificial Sequence
  Description of Artificial Sequence Synthetic primer
tttgattttacgccgtcctccaggtcg

SEQ ID NO: 708 - DNA, 32 bp, Artificial Sequence
  Description of Artificial Sequence Synthetic primer
tttgcggatgaagtaggtgcctatctttttgc

SEQ ID NO: 709 - DNA, 32 bp, Artificial Sequence
  Description of Artificial Sequence Synthetic primer
ttttatgcttaaagttggttttattggttggc

SEQ ID NO: 710 - DNA, 24 bp, Artificial Sequence
  Description of Artificial Sequence Synthetic primer
ttttgaaggtgatccgtgccaacg

SEQ ID NO: 711 - DNA, 29 bp, Artificial Sequence
  Description of Artificial Sequence Synthetic primer
aaactatttttttagctatactcgaacac

SEQ ID NO: 712 - DNA, 19 bp, Artificial Sequence
  Description of Artificial Sequence Synthetic primer
aacatagccttctccgtcc

SEQ ID NO: 713 - DNA, 21 bp, Artificial Sequence
  Description of Artificial Sequence Synthetic primer
aacttcgccttcggtcatgtt

SEQ ID NO: 714 - DNA, 17 bp, Artificial Sequence
  Description of Artificial Sequence Synthetic primer
aaggaggtgatccagcc

SEQ ID NO: 715 - DNA, 29 bp, Artificial Sequence
  Description of Artificial Sequence Synthetic primer
aatcgacgaccatcttggaaagatttctc

SEQ ID NO: 716 - DNA, 21 bp, Artificial Sequence
  Description of Artificial Sequence Synthetic primer
acaaaaggcacgccatcaccc

SEQ ID NO: 717 - DNA, 21 bp, Artificial Sequence
  Description of Artificial Sequence Synthetic primer
acaaaaggtacgccgtcaccc

SEQ ID NO: 718 - DNA, 18 bp, Artificial Sequence
  Description of Artificial Sequence Synthetic primer
acaacacgagcngacgac

SEQ ID NO: 719 - DNA, 18 bp, Artificial Sequence
  Description of Artificial Sequence Synthetic primer
acaacacgagctgacgac

SEQ ID NO: 720 - DNA, 18 bp, Artificial Sequence
  Description of Artificial Sequence Synthetic primer
acaacacgagctgacnac

SEQ ID NO: 721 - DNA, 18 bp, Artificial Sequence
  Description of Artificial Sequence Synthetic primer
acaacacgagctgncgac

SEQ ID NO: 722 - DNA, 18 bp, Artificial Sequence
  Description of Artificial Sequence Synthetic primer
acaacacgagntgacgac

SEQ ID NO: 723 - DNA, 18 bp, Artificial Sequence
  Description of Artificial Sequence Synthetic primer
acaacacganctnacgac

SEQ ID NO: 724 - DNA, 18 bp, Artificial Sequence
  Description of Artificial Sequence Synthetic primer
acaacacnanctnacgac

SEQ ID NO: 725 - DNA, 18 bp, Artificial Sequence
  Description of Artificial Sequence Synthetic primer
acaacacnanctnacnac

SEQ ID NO: 726 - DNA, 20 bp, Artificial Sequence
  Description of Artificial Sequence Synthetic primer
acaaccatgcaccacctgtc

SEQ ID NO: 727 - DNA, 12 bp, Artificial Sequence
  Description of Artificial Sequence Synthetic primer
acacgagctgac

SEQ ID NO: 728 - DNA, 28 bp, Artificial Sequence
  Description of Artificial Sequence Synthetic primer
accacttttaataaggtttgtagctaac

SEQ ID NO: 729 - DNA, 27 bp, Artificial Sequence
  Description of Artificial Sequence Synthetic primer
acctgcaatatctaatgcactcttacg

SEQ ID NO: 730 - DNA, 24 bp, Artificial Sequence
  Description of Artificial Sequence Synthetic primer
acctgcatccctaaacgtacttgc

SEQ ID NO: 731 - DNA, 22 bp, Artificial Sequence
  Description of Artificial Sequence Synthetic primer
accttgttacgacttcacccca

SEQ ID NO: 732 - DNA, 20 bp, Artificial Sequence
  Description of Artificial Sequence Synthetic primer
acgaactggatgtcgccgtt

SEQ ID NO: 733 - DNA, 18 bp, Artificial Sequence
  Description of Artificial Sequence Synthetic primer
acgacacgagctgacgac

SEQ ID NO: 734 - DNA, 18 bp, Artificial Sequence
  Description of Artificial Sequence Synthetic primer
acgacacgagctgacgac

SEQ ID NO: 735 - DNA, 20 bp, Artificial Sequence
  Description of Artificial Sequence Synthetic primer
acgagctgacgacagccatg

SEQ ID NO: 736 - DNA, 19 bp, Artificial Sequence
  Description of Artificial Sequence Synthetic primer
acgccatcaggccacgcat

SEQ ID NO: 737 - DNA, 21 bp, Artificial Sequence
  Description of Artificial Sequence Synthetic primer
acgcgggcatgcagagatgcc

SEQ ID NO: 738 - DNA, 18 bp, Artificial Sequence
  Description of Artificial Sequence Synthetic primer
acggcacgaggtagtcgc

SEQ ID NO: 739 - DNA, 20 bp, Artificial Sequence
  Description of Artificial Sequence Synthetic primer
acggttaccttgttacgact

SEQ ID NO: 740 - DNA, 20 bp, Artificial Sequence
  Description of Artificial Sequence Synthetic primer
acgtccttcatcgcctctga

SEQ ID NO: 741 - DNA, 27 bp, Artificial Sequence
  Description of Artificial Sequence Synthetic primer
acgtttttcgttttgaacgataatgct

SEQ ID NO: 742 - DNA, 17 bp, Artificial Sequence
  Description of Artificial Sequence Synthetic primer
actgctgcctcccgtag

SEQ ID NO: 743 - DNA, 20 bp, Artificial Sequence
  Description of Artificial Sequence Synthetic primer
acttagatgctttcagcggt

SEQ ID NO: 744 - DNA, 20 bp, Artificial Sequence
  Description of Artificial Sequence Synthetic primer
agacctcctgcgtgcaaagc

SEQ ID NO: 745 - DNA, 31 bp, Artificial Sequence
  Description of Artificial Sequence Synthetic primer
agataaagaatcacgaatatcaatttgtagc

SEQ ID NO: 746 - DNA, 22 bp, Artificial Sequence
  Description of Artificial Sequence Synthetic primer
agccgacatcgaggtgccaaac

SEQ ID NO: 747 - DNA, 28 bp, Artificial Sequence
  Description of Artificial Sequence Synthetic primer
agctgctagatgagcttctgccatggcc

SEQ ID NO: 748 - DNA, 23 bp, Artificial Sequence
  Description of Artificial Sequence Synthetic primer
aggatagatttatttcttgttcg

SEQ ID NO: 749 - DNA, 20 bp, Artificial Sequence
  Description of Artificial Sequence Synthetic primer
agtccatcccggtcctctcg

SEQ ID NO: 750 - DNA, 22 bp, Artificial Sequence
  Description of Artificial Sequence Synthetic primer
ataagccatgttctgttccatc

SEQ ID NO: 751 - DNA, 18 bp, Artificial Sequence
  Description of Artificial Sequence Synthetic primer
ataagccgggttctgtcg

SEQ ID NO: 752 - DNA, 26 bp, Artificial Sequence
  Description of Artificial Sequence Synthetic primer
atatgattatcattgaactgcggccg

SEQ ID NO: 753 - DNA, 19 bp, Artificial Sequence
  Description of Artificial Sequence Synthetic primer
atcccctgcttctgctgcc

SEQ ID NO: 754 - DNA, 30 bp, Artificial Sequence
  Description of Artificial Sequence Synthetic primer
attcaagagccatttcttttggtaaaccac

SEQ ID NO: 755 - DNA, 22 bp, Artificial Sequence
  Description of Artificial Sequence Synthetic primer
attgcccagaaatcaaatcatc

SEQ ID NO: 756 - DNA, 33 bp, Artificial Sequence
  Description of Artificial Sequence Synthetic primer
attgcttcttacttgcttagcataaattttcca

SEQ ID NO: 757 - DNA, 21 bp, Artificial Sequence
  Description of Artificial Sequence Synthetic primer
attgtagcacgtgtgtagccc

SEQ ID NO: 758 - DNA, 24 bp, Artificial Sequence
  Description of Artificial Sequence Synthetic primer
caagcggtttgcctcaaatagtca

SEQ ID NO: 759 - DNA, 22 bp, Artificial Sequence
  Description of Artificial Sequence Synthetic primer
caatctgctgacggatctgagc

SEQ ID NO: 760 - DNA, 15 bp, Artificial Sequence
  Description of Artificial Sequence Synthetic primer
caccgggcaggcgtc

SEQ ID NO: 761 - DNA, 20 bp, Artificial Sequence
  Description of Artificial Sequence Synthetic primer
cacggctaccttgttacgac

SEQ ID NO: 762 - DNA, 20 bp, Artificial Sequence
  Description of Artificial Sequence Synthetic primer
cagataaagaatcgctccag

SEQ ID NO: 763 - DNA, 25 bp, Artificial Sequence
  Description of Artificial Sequence Synthetic primer
catgacagccaagacctcacccacc

SEQ ID NO: 764 - DNA, 18 bp, Artificial Sequence
  Description of Artificial Sequence Synthetic primer
catgatggtcacaaccgg

SEQ ID NO: 765 - DNA, 20 bp, Artificial Sequence
  Description of Artificial Sequence Synthetic primer
ccaaacaccgccgtcgatat

SEQ ID NO: 766 - DNA, 26 bp, Artificial Sequence
  Description of Artificial Sequence Synthetic primer
ccaaccttttccacaacagaatcagc

SEQ ID NO: 767 - DNA, 27 bp, Artificial Sequence
  Description of Artificial Sequence Synthetic primer
ccaagtgctggtttaccccatggagta

SEQ ID NO: 768 - DNA, 23 bp, Artificial Sequence
  Description of Artificial Sequence Synthetic primer
ccacttttaataaggtttgtagc

SEQ ID NO: 769 - DNA, 27 bp, Artificial Sequence
  Description of Artificial Sequence Synthetic primer
ccagcagttactgtcccctcatctttg

SEQ ID NO: 770 - DNA, 28 bp, Artificial Sequence
  Description of Artificial Sequence Synthetic primer
ccataaggtcaccgtcaccattcaaagc

SEQ ID NO: 771 - DNA, 18 bp, Artificial Sequence
  Description of Artificial Sequence Synthetic primer
ccatgcagcacctgtctc

SEQ ID NO: 772 - DNA, 29 bp, Artificial Sequence
  Description of Artificial Sequence Synthetic primer
cccattttttcacgcatgctgaaaatatc

SEQ ID NO: 773 - DNA, 21 bp, Artificial Sequence
  Description of Artificial Sequence Synthetic primer
cccccgtcaattcctttgagt

SEQ ID NO: 774 - DNA, 24 bp, Artificial Sequence
  Description of Artificial Sequence Synthetic primer
ccctgtagtagaagaggtaaccac

SEQ ID NO: 775 - DNA, 21 bp, Artificial Sequence
  Description of Artificial Sequence Synthetic primer
ccgacaaggaatttcgctacc

SEQ ID NO: 776 - DNA, 23 bp, Artificial Sequence
  Description of Artificial Sequence Synthetic primer
ccgcggtcgaattgcatgccttc

SEQ ID NO: 777 - DNA, 17 bp, Artificial Sequence
  Description of Artificial Sequence Synthetic primer
ccggtcctctcgtacta

SEQ ID NO: 778 - DNA, 18 bp, Artificial Sequence
  Description of Artificial Sequence Synthetic primer
ccgtgctccatttttcag

SEQ ID NO: 779 - DNA, 25 bp, Artificial Sequence
  Description of Artificial Sequence Synthetic primer
cctacccaacgttcaccaagggcag

SEQ ID NO: 780 - DNA, 17 bp, Artificial Sequence
  Description of Artificial Sequence Synthetic primer
cctcctgcgtgcaaagc

SEQ ID NO: 781 - DNA, 20 bp, Artificial Sequence
  Description of Artificial Sequence Synthetic primer
cctgtagtagaagaggtaac

SEQ ID NO: 782 - DNA, 18 bp, Artificial Sequence
  Description of Artificial Sequence Synthetic primer
ccttctcccgaagttacg

SEQ ID NO: 783 - DNA, 20 bp, Artificial Sequence
  Description of Artificial Sequence Synthetic primer
ccttgttacgacttcacccc

SEQ ID NO: 784 - DNA, 24 bp, Artificial Sequence
  Description of Artificial Sequence Synthetic primer
cgaacggccagagtagtcaacacg

SEQ ID NO: 785 - DNA, 24 bp, Artificial Sequence
  Description of Artificial Sequence Synthetic primer
cgaacggcctgagtagtcaacacg

SEQ ID NO: 786 - DNA, 24 bp, Artificial Sequence
  Description of Artificial Sequence Synthetic primer
cgacttgacggttaacatttcctg

SEQ ID NO: 787 - DNA, 21 bp, Artificial Sequence
  Description of Artificial Sequence Synthetic primer
cgagttgcagactgcgatccg

SEQ ID NO: 788 - DNA, 21 bp, Artificial Sequence
  Description of Artificial Sequence Synthetic primer
cgagttgcagactgcgatccg

SEQ ID NO: 789 - DNA, 25 bp, Artificial Sequence
  Description of Artificial Sequence Synthetic primer
cgcaccatgcgtagagatgaagtac

SEQ ID NO: 790 - DNA, 25 bp, Artificial Sequence
  Description of Artificial Sequence Synthetic primer
cgcaccgtgggttgagatgaagtac

SEQ ID NO: 791 - DNA, 18 bp, Artificial Sequence
  Description of Artificial Sequence Synthetic primer
cgcatttcaccgctacac

SEQ ID NO: 792 - DNA, 20 bp, Artificial Sequence
  Description of Artificial Sequence Synthetic primer
cgcggtcggctcgttgatga

SEQ ID NO: 793 - DNA, 21 bp, Artificial Sequence
  Description of Artificial Sequence Synthetic primer
cggctgctggcacgaagttag

SEQ ID NO: 794 - DNA, 15 bp, Artificial Sequence
  Description of Artificial Sequence Synthetic primer
cggcttcaagacccc

SEQ ID NO: 795 - DNA, 24 bp, Artificial Sequence
  Description of Artificial Sequence Synthetic primer
cggtacgaactggatgtcgccgtt

SEQ ID NO: 796 - DNA, 15 bp, Artificial Sequence
  Description of Artificial Sequence Synthetic primer
cgtactccccaggcg

SEQ ID NO: 797 - DNA, 30 bp, Artificial Sequence
  Description of Artificial Sequence Synthetic primer
cgtataagctgcaccataagcttgtaatgc

SEQ ID NO: 798 - DNA, 21 bp, Artificial Sequence
  Description of Artificial Sequence Synthetic primer
cgtggactaccagggtatcta

SEQ ID NO: 799 - DNA, 21 bp, Artificial Sequence
  Description of Artificial Sequence Synthetic primer
ctatcggtcagtcaggagtat

SEQ ID NO: 800 - DNA, 22 bp, Artificial Sequence
  Description of Artificial Sequence Synthetic primer
cttctacatttttagccatcac

SEQ ID NO: 801 - DNA, 20 bp, Artificial Sequence
  Description of Artificial Sequence Synthetic primer
ctttacgcccagtaattccg

SEQ ID NO: 802 - DNA, 25 bp, Artificial Sequence
  Description of Artificial Sequence Synthetic primer
ctttcgctttctcgaactcaaccat

SEQ ID NO: 803 - DNA, 17 bp, Artificial Sequence
  Description of Artificial Sequence Synthetic primer
gaatatcaatttgtagc

SEQ ID NO: 804 - DNA, 27 bp, Artificial Sequence
  Description of Artificial Sequence Synthetic primer
gaccccaacctggccttttgtcgttga

SEQ ID NO: 805 - DNA, 19 bp, Artificial Sequence
  Description of Artificial Sequence Synthetic primer
gaccgttatagttacggcc

SEQ ID NO: 806 - DNA, 19 bp, Artificial Sequence
  Description of Artificial Sequence Synthetic primer
gacgggcggtgtgtacaag

SEQ ID NO: 807 - DNA, 19 bp, Artificial Sequence
  Description of Artificial Sequence Synthetic primer
gacgggcggtgtgtacaag

SEQ ID NO: 808 - DNA, 19 bp, Artificial Sequence
  Description of Artificial Sequence Synthetic primer
gacgggcggtgtgtacaag

SEQ ID NO: 809 - DNA, 21 bp, Artificial Sequence
  Description of Artificial Sequence Synthetic primer
gacgtcatccccaccttcctc

SEQ ID NO: 810 - DNA, 22 bp, Artificial Sequence
  Description of Artificial Sequence Synthetic primer
gacgtcatccccaccttcctcc

SEQ ID NO: 811 - DNA, 19 bp, Artificial Sequence
  Description of Artificial Sequence Synthetic primer
gagcatcagcgtgcgtgct

SEQ ID NO: 812 - DNA, 25 bp, Artificial Sequence
  Description of Artificial Sequence Synthetic primer
gagctgcgccaacgaataaatcgtc

SEQ ID NO: 813 - DNA, 29 bp, Artificial Sequence
  Description of Artificial Sequence Synthetic primer
gattggcgataaagtgatattttctaaaa

SEQ ID NO: 814 - DNA, 26 bp, Artificial Sequence
  Description of Artificial Sequence Synthetic primer
gcccaccagaaagactagcaggataa

SEQ ID NO: 815 - DNA, 21 bp, Artificial Sequence
  Description of Artificial Sequence Synthetic primer
gccgtccatctgagcagcacc

SEQ ID NO: 816 - DNA, 21 bp, Artificial Sequence
  Description of Artificial Sequence Synthetic primer
gccgtccatttgagcagcacc

SEQ ID NO: 817 - DNA, 19 bp, Artificial Sequence
  Description of Artificial Sequence Synthetic primer
gccttgcgaccgtactccc

SEQ ID NO: 818 - DNA, 18 bp, Artificial Sequence
  Description of Artificial Sequence Synthetic primer
gcgaccgtactccccagg

SEQ ID NO: 819 - DNA, 19 bp, Artificial Sequence
  Description of Artificial Sequence Synthetic primer
gcgctccacgtcttcacgc

SEQ ID NO: 820 - DNA, 19 bp, Artificial Sequence
  Description of Artificial Sequence Synthetic primer
gcgtgacaggcaggtattc

SEQ ID NO: 821 - DNA, 28 bp, Artificial Sequence
  Description of Artificial Sequence Synthetic primer
gcgtgacgaccttcttgaattgtaatca

SEQ ID NO: 822 - DNA, 24 bp, Artificial Sequence
  Description of Artificial Sequence Synthetic primer
gcgttccacagcttgttgcagaag

SEQ ID NO: 823 - DNA, 18 bp, Artificial Sequence
  Description of Artificial Sequence Synthetic primer
gctgctggcacggagtta

SEQ ID NO: 824 - DNA, 26 bp, Artificial Sequence
  Description of Artificial Sequence Synthetic primer
gctgctttgatggctgaatccccttc

SEQ ID NO: 825 - DNA, 21 bp, Artificial Sequence
  Description of Artificial Sequence Synthetic primer
gctggattcgcctttgctacg

SEQ ID NO: 826 - DNA, 20 bp, Artificial Sequence
  Description of Artificial Sequence Synthetic primer
gcttacacacccggcctatc

SEQ ID NO: 827 - DNA, 23 bp, Artificial Sequence
  Description of Artificial Sequence Synthetic primer
ggaatttaccagcgatagacacc

SEQ ID NO: 828 - DNA, 30 bp, Artificial Sequence
  Description of Artificial Sequence Synthetic primer
ggataattggtcgtaacaagggatagtgag

SEQ ID NO: 829 - DNA, 25 bp, Artificial Sequence
  Description of Artificial Sequence Synthetic primer
ggcatcaccatttccttgtccttcg

SEQ ID NO: 830 - DNA, 18 bp, Artificial Sequence
  Description of Artificial Sequence Synthetic primer
ggccgtactccccaggcg

SEQ ID NO: 831 - DNA, 20 bp, Artificial Sequence
  Description of Artificial Sequence Synthetic primer
ggcgcttgtacttaccgcac

SEQ ID NO: 832 - DNA, 25 bp, Artificial Sequence
  Description of Artificial Sequence Synthetic primer
gggtctacacctgcacttgcataac

SEQ ID NO: 833 - DNA, 16 bp, Artificial Sequence
  Description of Artificial Sequence Synthetic primer
gggtttccccattcgg

SEQ ID NO: 834 - DNA, 20 bp, Artificial Sequence
  Description of Artificial Sequence Synthetic primer
ggtaacccttgtctttgaat

SEQ ID NO: 835 - DNA, 19 bp, Artificial Sequence
  Description of Artificial Sequence Synthetic primer
ggtaaggttcttcgcgttg

SEQ ID NO: 836 - DNA, 28 bp, Artificial Sequence
  Description of Artificial Sequence Synthetic primer
ggtataacgcatcgcagcaaaagattta

SEQ ID NO: 837 - DNA, 28 bp, Artificial Sequence
  Description of Artificial Sequence Synthetic primer
gtaacccttgtctttgaattgtatttgc

SEQ ID NO: 838 - DNA, 22 bp, Artificial Sequence
  Description of Artificial Sequence Synthetic primer
gtaagccatgttttgttccatc

SEQ ID NO: 839 - DNA, 22 bp, Artificial Sequence
  Description of Artificial Sequence Synthetic primer
gtatctaatcctgtttgctccc

SEQ ID NO: 840 - DNA, 27 bp, Artificial Sequence
  Description of Artificial Sequence Synthetic primer
gtccgacttgacggtcaacatttcctg

SEQ ID NO: 841 - DNA, 18 bp, Artificial Sequence
  Description of Artificial Sequence Synthetic primer
gtgcgccctttctaactt

SEQ ID NO: 842 - DNA, 22 bp, Artificial Sequence
  Description of Artificial Sequence Synthetic primer
gtgctggtttaccccatggagt

SEQ ID NO: 843 - DNA, 21 bp, Artificial Sequence
  Description of Artificial Sequence Synthetic primer
gttcaaatgcctggataccca

SEQ ID NO: 844 - DNA, 24 bp, Artificial Sequence
  Description of Artificial Sequence Synthetic primer
gttgtcaccaggcattaccatttc

SEQ ID NO: 845 - DNA, 24 bp, Artificial Sequence
  Description of Artificial Sequence Synthetic primer
gttgtcgccaggcataaccatttc

SEQ ID NO: 846 - DNA, 24 bp, Artificial Sequence
  Description of Artificial Sequence Synthetic primer
gtttcatgcttagatgctttcagc

SEQ ID NO: 847 - DNA, 25 bp, Artificial Sequence
  Description of Artificial Sequence Synthetic primer
gtttttcgttgcgtacgatgatgtc

SEQ ID NO: 848 - DNA, 27 bp, Artificial Sequence
  Description of Artificial Sequence Synthetic primer
taaacgtccgataccaatggttcgctc

SEQ ID NO: 849 - DNA, 30 bp, Artificial Sequence
  Description of Artificial Sequence Synthetic primer
taaactatttttttagctatactcgaacac

SEQ ID NO: 850 - DNA, 28 bp, Artificial Sequence
  Description of Artificial Sequence Synthetic primer
taaagacaccgctgggtttaaatgtgca

SEQ ID NO: 851 - DNA, 27 bp, Artificial Sequence
  Description of Artificial Sequence Synthetic primer
taaagagacgtttggtagttcatttgc

SEQ ID NO: 852 - DNA, 33 bp, Artificial Sequence
  Description of Artificial Sequence Synthetic primer
taaaggatagcggtaactaaatggctgagccat

SEQ ID NO: 853 - DNA, 26 bp, Artificial Sequence
  Description of Artificial Sequence Synthetic primer
taaatgcacttgcttcagggccatat

SEQ ID NO: 854 - DNA, 31 bp, Artificial Sequence
  Description of Artificial Sequence Synthetic primer
taaattccgcaaagactttggcattaggtgt

SEQ ID NO: 855 - DNA, 29 bp, Artificial Sequence
  Description of Artificial Sequence Synthetic primer
taacaaatcccgtctgagttcctcttgca

SEQ ID NO: 856 - DNA, 28 bp, Artificial Sequence
  Description of Artificial Sequence Synthetic primer
taacaacgttaccttcgcgatccactaa

SEQ ID NO: 857 - DNA, 23 bp, Artificial Sequence
  Description of Artificial Sequence Synthetic primer
taaccatttcgcgtaagattcaa

SEQ ID NO: 858 - DNA, 29 bp, Artificial Sequence
  Description of Artificial Sequence Synthetic primer
taaccaccccaagatttatctttttgcca

SEQ ID NO: 859 - DNA, 23 bp, Artificial Sequence
  Description of Artificial Sequence Synthetic primer
taaccatttcgcgtaagattcaa

SEQ ID NO: 860 - DNA, 39 bp, Artificial Sequence
  Description of Artificial Sequence Synthetic primer
taacccttgtctttgaattgtatttgcaattaatcctgg

SEQ ID NO: 861 - DNA, 29 bp, Artificial Sequence
  Description of Artificial Sequence Synthetic primer
taaccgtttccaaaggtactgtattttgt

SEQ ID NO: 862 - DNA, 34 bp, Artificial Sequence
  Description of Artificial Sequence Synthetic primer
taaccgtttccaaaggtactgtattttgtttacc

SEQ ID NO: 863 - DNA, 26 bp, Artificial Sequence
  Description of Artificial Sequence Synthetic primer
taactcctcttccttcaacaggtgga

SEQ ID NO: 864 - DNA, 29 bp, Artificial Sequence
  Description of Artificial Sequence Synthetic primer
taactgacccaaagctgaaagctttactg

SEQ ID NO: 865 - DNA, 33 bp, Artificial Sequence
  Description of Artificial Sequence Synthetic primer
taagacaaggttttgtggattttttagcttgtt

SEQ ID NO: 866 - DNA, 26 bp, Artificial Sequence
  Description of Artificial Sequence Synthetic primer
taagagtgatgcgggctggttcaaca

SEQ ID NO: 867 - DNA, 25 bp, Artificial Sequence
  Description of Artificial Sequence Synthetic primer
taagcaatacctttacttgcaccac

SEQ ID NO: 868 - DNA, 27 bp, Artificial Sequence
  Description of Artificial Sequence Synthetic primer
taagcaatacctttacttgcaccacct

SEQ ID NO: 869 - DNA, 28 bp, Artificial Sequence
  Description of Artificial Sequence Synthetic primer
taagcaatacctttacttgcaccacctg

SEQ ID NO: 870 - DNA, 31 bp, Artificial Sequence
  Description of Artificial Sequence Synthetic primer
taagcaccatataagtctacttttttccctt

SEQ ID NO: 871 - DNA, 27 bp, Artificial Sequence
  Description of Artificial Sequence Synthetic primer
taagccagcaagagctgtatagttcca

SEQ ID NO: 872 - DNA, 26 bp, Artificial Sequence
  Description of Artificial Sequence Synthetic primer
taagctcccgtatcttgagtcgcctc

SEQ ID NO: 873 - DNA, 23 bp, Artificial Sequence
  Description of Artificial Sequence Synthetic primer
taagttaccttgcccgtcaacca

SEQ ID NO: 874 - DNA, 27 bp, Artificial Sequence
  Description of Artificial Sequence Synthetic primer
taagttccttcgctagtatgttggctt

SEQ ID NO: 875 - DNA, 30 bp, Artificial Sequence
  Description of Artificial Sequence Synthetic primer
taatcgacgaccatcttggaaagatttctc

SEQ ID NO: 876 - DNA, 23 bp, Artificial Sequence
  Description of Artificial Sequence Synthetic primer
taatctggctgcggaagtgaaat

SEQ ID NO: 877 - DNA, 25 bp, Artificial Sequence
  Description of Artificial Sequence Synthetic primer
taatctggctgcggaagtgaaatcg

SEQ ID NO: 878 - DNA, 31 bp, Artificial Sequence
  Description of Artificial Sequence Synthetic primer
taatgccgggtagtgcaatccattcttctag

SEQ ID NO: 879 - DNA, 24 bp, Artificial Sequence
  Description of Artificial Sequence Synthetic primer
taatgcgatactggcctgcaagtc

SEQ ID NO: 880 - DNA, 33 bp, Artificial Sequence
  Description of Artificial Sequence Synthetic primer
tacaaccttcggataatcaggatgagaattaat

SEQ ID NO: 881 - DNA, 27 bp, Artificial Sequence
  Description of Artificial Sequence Synthetic primer
tacaacgtgataaacacgaccagaagc

SEQ ID NO: 882 - DNA, 35 bp, Artificial Sequence
  Description of Artificial Sequence Synthetic primer
tacaactggttcaaaaacattaagctgtaattgtc

SEQ ID NO: 883 - DNA, 32 bp, Artificial Sequence
  Description of Artificial Sequence Synthetic primer
tacagctttaaagccagcaaaatgaattacag

SEQ ID NO: 884 - DNA, 22 bp, Artificial Sequence
  Description of Artificial Sequence Synthetic primer
tacaggagcagcaggcttcaag

SEQ ID NO: 885 - DNA, 26 bp, Artificial Sequence
  Description of Artificial Sequence Synthetic primer
tacatcgtttcgcccaagatcaatca

SEQ ID NO: 886 - DNA, 33 bp, Artificial Sequence
  Description of Artificial Sequence Synthetic primer
tacatctccttcgatagaaatttcattgctatc

SEQ ID NO: 887 - DNA, 25 bp, Artificial Sequence
  Description of Artificial Sequence Synthetic primer
taccaaagcgtgcacgatagttgag

SEQ ID NO: 888 - DNA, 27 bp, Artificial Sequence
  Description of Artificial Sequence Synthetic primer
taccatctacccaaacattagcaccaa

SEQ ID NO: 889 - DNA, 22 bp, Artificial Sequence
  Description of Artificial Sequence Synthetic primer
taccccagttcccctgaccttc

SEQ ID NO: 890 - DNA, 27 bp, Artificial Sequence
  Description of Artificial Sequence Synthetic primer
taccggaagcaccagcgacattaatag

SEQ ID NO: 891 - DNA, 27 bp, Artificial Sequence
  Description of Artificial Sequence Synthetic primer
tacctgcattaatcgcttgttcatcaa

SEQ ID NO: 892 - DNA, 22 bp, Artificial Sequence
  Description of Artificial Sequence Synthetic primer
taccttaccgccaaagctgtct

SEQ ID NO: 893 - DNA, 24 bp, Artificial Sequence
  Description of Artificial Sequence Synthetic primer
taccttaggaccgttatagttacg

SEQ ID NO: 894 - DNA, 24 bp, Artificial Sequence
  Description of Artificial Sequence Synthetic primer
taccttttccacaacagaatcagc

SEQ ID NO: 895 - DNA, 21 bp, Artificial Sequence
  Description of Artificial Sequence Synthetic primer
tacgagctgacgacagccatg

SEQ ID NO: 896 - DNA, 23 bp, Artificial Sequence
  Description of Artificial Sequence Synthetic primer
tacgagctgacgacagccatgca

SEQ ID NO: 897 - DNA, 22 bp, Artificial Sequence
  Description of Artificial Sequence Synthetic primer
tacgcattactcacccgtccgc

SEQ ID NO: 898 - DNA, 20 bp, Artificial Sequence
  Description of Artificial Sequence Synthetic primer
tacgccatcaggccacgcat

SEQ ID NO: 899 - DNA, 27 bp, Artificial Sequence
  Description of Artificial Sequence Synthetic primer
tacgctaagccacgtccatatttatca

SEQ ID NO: 900 - DNA, 35 bp, Artificial Sequence
  Description of Artificial Sequence Synthetic primer
tacgtatgtaaattccgcaaagactttggcattag

SEQ ID NO: 901 - DNA, 30 bp, Artificial Sequence
  Description of Artificial Sequence Synthetic primer
tacgtcgcctttaacttggttatattcagc

SEQ ID NO: 902 - DNA, 31 bp, Artificial Sequence
  Description of Artificial Sequence Synthetic primer
tacgttctacgatttcttcatcaggtacatc

SEQ ID NO: 903 - DNA, 24 bp, Artificial Sequence
  Description of Artificial Sequence Synthetic primer
tacgtttgtatcttctgcagaacc

SEQ ID NO: 904 - DNA, 29 bp, Artificial Sequence
  Description of Artificial Sequence Synthetic primer
tacacctggtttcgttttgatgatttgta

SEQ ID NO: 905 - DNA, 24 bp, Artificial Sequence
  Description of Artificial Sequence Synthetic primer
tactagacgacgggtcaggtaacc

SEQ ID NO: 906 - DNA, 32 bp, Artificial Sequence
  Description of Artificial Sequence Synthetic primer
tacttcagcttcgtccaataaaaaatcacaat

SEQ ID NO: 907 - DNA, 31 bp, Artificial Sequence
  Description of Artificial Sequence Synthetic primer
tactttaaggggctatctttaccatgaacct

SEQ ID NO: 908 - DNA, 27 bp, Artificial Sequence
  Description of Artificial Sequence Synthetic primer
tagagagtagccatcttcaccgttgtc

SEQ ID NO: 909 - DNA, 23 bp, Artificial Sequence
  Description of Artificial Sequence Synthetic primer
tagcaccaatcaccctttcctgt

SEQ ID NO: 910 - DNA, 28 bp, Artificial Sequence
  Description of Artificial Sequence Synthetic primer
tagcagcaaaagttatcacacctgcagt

SEQ ID NO: 911 - DNA, 25 bp, Artificial Sequence
  Description of Artificial Sequence Synthetic primer
tagcagctagctcgtaaccagtgta

SEQ ID NO: 912 - DNA, 31 bp, Artificial Sequence
  Description of Artificial Sequence Synthetic primer
tagccatacgtaccattgcttcataaataga

SEQ ID NO: 913 - DNA, 22 bp, Artificial Sequence
  Description of Artificial Sequence Synthetic primer
tagcccagctgtttgagcaact

SEQ ID NO: 914 - DNA, 20 bp, Artificial Sequence
  Description of Artificial Sequence Synthetic primer
tagccgcggtcgaattgcat

SEQ ID NO: 915 - DNA, 25 bp, Artificial Sequence
  Description of Artificial Sequence Synthetic primer
tagccttggcaacatcagcaaaact

SEQ ID NO: 916 - DNA, 24 bp, Artificial Sequence
  Description of Artificial Sequence Synthetic primer
tagccttttctccggcgtagatct

SEQ ID NO: 917 - DNA, 34 bp, Artificial Sequence
  Description of Artificial Sequence Synthetic primer
tagcgatttctactcctagagttgaaatttcagg

SEQ ID NO: 918 - DNA, 29 bp, Artificial Sequence
  Description of Artificial Sequence Synthetic primer
tagctgctagatgagcttctgccatggcc

SEQ ID NO: 919 - DNA, 23 bp, Artificial Sequence
  Description of Artificial Sequence Synthetic primer
taggatgaaagcattccgctggc

SEQ ID NO: 920 - DNA, 28 bp, Artificial Sequence
  Description of Artificial Sequence Synthetic primer
taggatgagcattatcagggaaagaatc

SEQ ID NO: 921 - DNA, 23 bp, Artificial Sequence
  Description of Artificial Sequence Synthetic primer
taggatttttccacggcggcatc

SEQ ID NO: 922 - DNA, 31 bp, Artificial Sequence
  Description of Artificial Sequence Synthetic primer
taggcataaccatttcagtaccttctggtaa

SEQ ID NO: 923 - DNA, 28 bp, Artificial Sequence
  Description of Artificial Sequence Synthetic primer
tagtatcaccacgtacacccggatcagt

SEQ ID NO: 924 - DNA, 28 bp, Artificial Sequence
  Description of Artificial Sequence Synthetic primer
tagtatcaccacgtacnccnggatcagt

SEQ ID NO: 925 - DNA, 32 bp, Artificial Sequence
  Description of Artificial Sequence Synthetic primer
tagtcctttctgaattttaccatcaaaggtac

SEQ ID NO: 926 - DNA, 32 bp, Artificial Sequence
  Description of Artificial Sequence Synthetic primer
tagtcttttggaacaccgtctttaattaaagt

SEQ ID NO: 927 - DNA, 30 bp, Artificial Sequence
  Description of Artificial Sequence Synthetic primer
tagtgttgtacctccatatagacattcaga

SEQ ID NO: 928 - DNA, 29 bp, Artificial Sequence
  Description of Artificial Sequence Synthetic primer
tagttgaagttgcactatatactgttgga

SEQ ID NO: 929 - DNA, 23 bp, Artificial Sequence
  Description of Artificial Sequence Synthetic primer
tataacgcacatcgtcagggtga

SEQ ID NO: 930 - DNA, 26 bp, Artificial Sequence
  Description of Artificial Sequence Synthetic primer
tatagcaccatccatctgagcggcac

SEQ ID NO: 931 - DNA, 29 bp, Artificial Sequence
  Description of Artificial Sequence Synthetic primer
tatatgaacaataccagttccttctgagt

SEQ ID NO: 932 - DNA, 27 bp, Artificial Sequence
  Description of Artificial Sequence Synthetic primer
tatatgattatcattgaactgcggccg

SEQ ID NO: 933 - DNA, 28 bp, Artificial Sequence
  Description of Artificial Sequence Synthetic primer
tatccattgaaccaaagttaccttggcc

SEQ ID NO: 934 - DNA, 20 bp, Artificial Sequence
  Description of Artificial Sequence Synthetic primer
tatcccctgcttctgctgcc

SEQ ID NO: 935 - DNA, 28 bp, Artificial Sequence
  Description of Artificial Sequence Synthetic primer
tatcgacagatccaaagttaccatgccc

SEQ ID NO: 936 - DNA, 27 bp, Artificial Sequence
  Description of Artificial Sequence Synthetic primer
tatggtctatttcaatggcagttacga

SEQ ID NO: 937 - DNA, 24 bp, Artificial Sequence
  Description of Artificial Sequence Synthetic primer
tatgtgctcacgagtttgcggcat

SEQ ID NO: 938 - DNA, 28 bp, Artificial Sequence
  Description of Artificial Sequence Synthetic primer
tatgtgtagttgagcttactacatgagc

SEQ ID NO: 939 - DNA, 25 bp, Artificial Sequence
  Description of Artificial Sequence Synthetic primer
tattcttcgttactcatgccataca

SEQ ID NO: 940 - DNA, 23 bp, Artificial Sequence
  Description of Artificial Sequence Synthetic primer
tattgcccagaaatcaaatcatc

SEQ ID NO: 941 - DNA, 30 bp, Artificial Sequence
  Description of Artificial Sequence Synthetic primer
tattgcggatcaccatgatgatattcttgc

SEQ ID NO: 942 - DNA, 31 bp, Artificial Sequence
  Description of Artificial Sequence Synthetic primer
tattgctttttttgctatgcttcttggacat

SEQ ID NO: 943 - DNA, 24 bp, Artificial Sequence
  Description of Artificial Sequence Synthetic primer
tattggaaataccggcagcatctc

SEQ ID NO: 944 - DNA, 30 bp, Artificial Sequence
  Description of Artificial Sequence Synthetic primer
tatttgggtttcattccactcagattctgg

SEQ ID NO: 945 - DNA, 32 bp, Artificial Sequence
  Description of Artificial Sequence Synthetic primer
tcaaaaacaaagaattcattttctggtccaaa

SEQ ID NO: 946 - DNA, 34 bp, Artificial Sequence
  Description of Artificial Sequence Synthetic primer
tcaaaacgcatttttacatcttcgttaaaggcta

SEQ ID NO: 947 - DNA, 29 bp, Artificial Sequence
  Description of Artificial Sequence Synthetic primer
tcaaaacttgctctagaccatttaactcc

SEQ ID NO: 948 - DNA, 30 bp, Artificial Sequence
  Description of Artificial Sequence Synthetic primer
tcaaaatcttttgattcgatcatacgagac

SEQ ID NO: 949 - DNA, 27 bp, Artificial Sequence
  Description of Artificial Sequence Synthetic primer
tcaaacgatccgcatcaccatcaaaag

SEQ ID NO: 950 - DNA, 30 bp, Artificial Sequence
  Description of Artificial Sequence Synthetic primer
tcaaagaaccagcacctaattcatcattta

SEQ ID NO: 951 - DNA, 30 bp, Artificial Sequence
  Description of Artificial Sequence Synthetic primer
tcaaagaacccgcacctaattcatcattta

SEQ ID NO: 952 - DNA, 26 bp, Artificial Sequence
  Description of Artificial Sequence Synthetic primer
tcaacaacacctccttattcccactc

SEQ ID NO: 953 - DNA, 29 bp, Artificial Sequence
  Description of Artificial Sequence Synthetic primer
tcaacaatcagatagatgtcagacgcatg

SEQ ID NO: 954 - DNA, 28 bp, Artificial Sequence
  Description of Artificial Sequence Synthetic primer
tcaacaccagcgttacctaaagtacctt

SEQ ID NO: 955 - DNA, 35 bp, Artificial Sequence
  Description of Artificial Sequence Synthetic primer
tcaactggttcaaaaacattaagttgtaattgtcc

SEQ ID NO: 956 - DNA, 28 bp, Artificial Sequence
  Description of Artificial Sequence Synthetic primer
tcaacttctgccattaaaagtaatgcca

SEQ ID NO: 957 - DNA, 24 bp, Artificial Sequence
  Description of Artificial Sequence Synthetic primer
tcaagcgatctacccgcattacaa

SEQ ID NO: 958 - DNA, 30 bp, Artificial Sequence
  Description of Artificial Sequence Synthetic primer
tcaagcgccatctctttcggtaatccacat

SEQ ID NO: 959 - DNA, 30 bp, Artificial Sequence
  Description of Artificial Sequence Synthetic primer
tcaagcgccatttcttttggtaaaccacat

SEQ ID NO: 960 - DNA, 31 bp, Artificial Sequence
  Description of Artificial Sequence Synthetic primer
tcaagctatatgctacaactggttcaaaaac

SEQ ID NO: 961 - DNA, 30 bp, Artificial Sequence
  Description of Artificial Sequence Synthetic primer
tcaagctctacaccataaaaaaagctctca

SEQ ID NO: 962 - DNA, 28 bp, Artificial Sequence
  Description of Artificial Sequence Synthetic primer
tcaaggttctcaccgtttaccttaggag

SEQ ID NO: 963 - DNA, 32 bp, Artificial Sequence
  Description of Artificial Sequence Synthetic primer
tcaagtgcttttacttctataggtttaagctc

SEQ ID NO: 964 - DNA, 30 bp, Artificial Sequence
  Description of Artificial Sequence Synthetic primer
tcaatacagagtctacacttggcttaggat

SEQ ID NO: 965 - DNA, 25 bp, Artificial Sequence
  Description of Artificial Sequence Synthetic primer
tcaatctcgactttttgtgccggta

SEQ ID NO: 966 - DNA, 28 bp, Artificial Sequence
  Description of Artificial Sequence Synthetic primer
tcacaaggaccattataatcaatgccaa

SEQ ID NO: 967 - DNA, 23 bp, Artificial Sequence
  Description of Artificial Sequence Synthetic primer
tcacaccaagtagtgcaaggatc

SEQ ID NO: 968 - DNA, 29 bp, Artificial Sequence
  Description of Artificial Sequence Synthetic primer
tcacacctgtaagtgagaaaaaggttgat

SEQ ID NO: 969 - DNA, 34 bp, Artificial Sequence
  Description of Artificial Sequence Synthetic primer
tcacaggttctacttcatcaataatttccattgc

SEQ ID NO: 970 - DNA, 34 bp, Artificial Sequence
  Description of Artificial Sequence Synthetic primer
tcaccagcttcagcgtagtctaataatttacgga

SEQ ID NO: 971 - DNA, 22 bp, Artificial Sequence
  Description of Artificial Sequence Synthetic primer
tcaccatgcgcccgttcacata

SEQ ID NO: 972 - DNA, 37 bp, Artificial Sequence
  Description of Artificial Sequence Synthetic primer
tcaccgataaataaaatacctaaagttaatgccattg

SEQ ID NO: 973 - DNA, 29 bp, Artificial Sequence
  Description of Artificial Sequence Synthetic primer
tcacctacagctttaaagccagcaaaatg

SEQ ID NO: 974 - DNA, 28 bp, Artificial Sequence
  Description of Artificial Sequence Synthetic primer
tcacgatacctgcatcatcaaattggtt

SEQ ID NO: 975 - DNA, 32 bp, Artificial Sequence
  Description of Artificial Sequence Synthetic primer
tcacgatctaaatttggataagccataggaaa

SEQ ID NO: 976 - DNA, 24 bp, Artificial Sequence
  Description of Artificial Sequence Synthetic primer
tcacgcgacgagtgccatccattg

SEQ ID NO: 977 - DNA, 22 bp, Artificial Sequence
  Description of Artificial Sequence Synthetic primer
tcacgcgcatcatcaccagtca

SEQ ID NO: 978 - DNA, 18 bp, Artificial Sequence
  Description of Artificial Sequence Synthetic primer
tcacgggccagctcgtct

SEQ ID NO: 979 - DNA, 28 bp, Artificial Sequence
  Description of Artificial Sequence Synthetic primer
tcacgtcgtccgacttcacggtcagcat

SEQ ID NO: 980 - DNA, 26 bp, Artificial Sequence
  Description of Artificial Sequence Synthetic primer
tcagaatcgatgccaaatgcgtcatc

SEQ ID NO: 981 - DNA, 30 bp, Artificial Sequence
  Description of Artificial Sequence Synthetic primer
tcagatataaatggaacaaatggagccact

SEQ ID NO: 982 - DNA, 32 bp, Artificial Sequence
  Description of Artificial Sequence Synthetic primer
tcagcgtagtctaataatttacggaacatttc

SEQ ID NO: 983 - DNA, 25 bp, Artificial Sequence
  Description of Artificial Sequence Synthetic primer
tcagctgttaacggcttcaagaccc

SEQ ID NO: 984 - DNA, 29 bp, Artificial Sequence
  Description of Artificial Sequence Synthetic primer
tcaggtatgaaacacgattagtcctttct

SEQ ID NO: 985 - DNA, 29 bp, Artificial Sequence
  Description of Artificial Sequence Synthetic primer
tcagtttgcacttcaaaagaaattgtgtt

SEQ ID NO: 986 - DNA, 29 bp, Artificial Sequence
  Description of Artificial Sequence Synthetic primer
tcataactagcatttgtgctttgaatgct

SEQ ID NO: 987 - DNA, 31 bp, Artificial Sequence
  Description of Artificial Sequence Synthetic primer
tcataagggttgcgttgcagattatctttac

SEQ ID NO: 988 - DNA, 26 bp, Artificial Sequence
  Description of Artificial Sequence Synthetic primer
tcatctggtttaggatctggttgact

SEQ ID NO: 989 - DNA, 25 bp, Artificial Sequence
  Description of Artificial Sequence Synthetic primer
tcatctgtggtatggcgggtaagtt

SEQ ID NO: 990 - DNA, 26 bp, Artificial Sequence
  Description of Artificial Sequence Synthetic primer
tcatgacagccaagacctcacccacc

SEQ ID NO: 991 - DNA, 31 bp, Artificial Sequence
  Description of Artificial Sequence Synthetic primer
tcatgatagaactacctggttgcatttttgg

SEQ ID NO: 992 - DNA, 28 bp, Artificial Sequence
  Description of Artificial Sequence Synthetic primer
tcatgtgctaatgttactgctggatctg

SEQ ID NO: 993 - DNA, 31 bp, Artificial Sequence
  Description of Artificial Sequence Synthetic primer
tcattaggtaaaatgtctggacatgatccaa

SEQ ID NO: 994 - DNA, 28 bp, Artificial Sequence
  Description of Artificial Sequence Synthetic primer
tcatttatttcttcgcttttctcgctac

SEQ ID NO: 995 - DNA, 20 bp, Artificial Sequence
  Description of Artificial Sequence Synthetic primer
tcatttgtgctttgaatgct

SEQ ID NO: 996 - DNA, 28 bp, Artificial Sequence
  Description of Artificial Sequence Synthetic primer
tccaaacgatctgcatcaccatcaaaag

SEQ ID NO: 997 - DNA, 29 bp, Artificial Sequence
  Description of Artificial Sequence Synthetic primer
tccaacccagaaccacatactttattcac

SEQ ID NO: 998 - DNA, 27 bp, Artificial Sequence
  Description of Artificial Sequence Synthetic primer
tccaaccttttccacaacagaatcagc

SEQ ID NO: 999 - DNA, 24 bp, Artificial Sequence
  Description of Artificial Sequence Synthetic primer
tccaagtgctggtttaccccatgg

SEQ ID NO: 1000 - DNA, 26 bp, Artificial Sequence
  Description of Artificial Sequence Synthetic primer
tccaagtgctggtttaccccatggag

SEQ ID NO: 1001 - DNA, 28 bp, Artificial Sequence
  Description of Artificial Sequence Synthetic primer
tccaagtttgacttaaacgtaccatcgc

SEQ ID NO: 1002 - DNA, 32 bp, Artificial Sequence
  Description of Artificial Sequence Synthetic primer
tccacactggattgtaatttaccttgttcttt

SEQ ID NO: 1003 - DNA, 26 bp, Artificial Sequence
  Description of Artificial Sequence Synthetic primer
tccaccacctcaaagaccatgtggtg

SEQ ID NO: 1004 - DNA, 23 bp, Artificial Sequence
  Description of Artificial Sequence Synthetic primer
tccagcaggttctgacggaaacg

SEQ ID NO: 1005 - DNA, 28 bp, Artificial Sequence
  Description of Artificial Sequence Synthetic primer
tccagcagttactgtcccctcatctttg

SEQ ID NO: 1006 - DNA, 30 bp, Artificial Sequence
  Description of Artificial Sequence Synthetic primer
tccaggcattaccatttctactccttctgg

SEQ ID NO: 1007 - DNA, 29 bp, Artificial Sequence
  Description of Artificial Sequence Synthetic primer
tccataaggtcaccgtcaccattcaaagc

SEQ ID NO: 1008 - DNA, 35 bp, Artificial Sequence
  Description of Artificial Sequence Synthetic primer
tccatacctttatgcaacttngtatcaacnggaat

SEQ ID NO: 1009 - DNA, 28 bp, Artificial Sequence
  Description of Artificial Sequence Synthetic primer
tccatattgttgcataaaacctgttggc

SEQ ID NO: 1010 - DNA, 27 bp, Artificial Sequence
  Description of Artificial Sequence Synthetic primer
tccatccatagaaccaaagttaccttg

SEQ ID NO: 1011 - DNA, 26 bp, Artificial Sequence
  Description of Artificial Sequence Synthetic primer
tccatcgcagtcacgtttactgttgg

SEQ ID NO: 1012 - DNA, 32 bp, Artificial Sequence
  Description of Artificial Sequence Synthetic primer
tccatcgccagtttttgcataatcgctaaaaa

SEQ ID NO: 1013 - DNA, 33 bp, Artificial Sequence
  Description of Artificial Sequence Synthetic primer
tccatctgttaaaccatcatataccatgctatc

SEQ ID NO: 1014 - DNA, 26 bp, Artificial Sequence
  Description of Artificial Sequence Synthetic primer
tccatttccgacacgtcgttgatcac

SEQ ID NO: 1015 - DNA, 27 bp, Artificial Sequence
  Description of Artificial Sequence Synthetic primer
tcccaatctaacttccacataccatct

SEQ ID NO: 1016 - DNA, 29 bp, Artificial Sequence
  Description of Artificial Sequence Synthetic primer
tcccaatcttttgattcgatcatacgaga

SEQ ID NO: 1017 - DNA, 27 bp, Artificial Sequence
  Description of Artificial Sequence Synthetic primer
tcccatacctatggcgataactgtcat

SEQ ID NO: 1018 - DNA, 30 bp, Artificial Sequence
  Description of Artificial Sequence Synthetic primer
tcccattttttcacgcatgctgaaaatatc

SEQ ID NO: 1019 - DNA, 15 bp, Artificial Sequence
  Description of Artificial Sequence Synthetic primer
tccccaccttcctcc

SEQ ID NO: 1020 - DNA, 25 bp, Artificial Sequence
  Description of Artificial Sequence Synthetic primer
tccccatctccgcaaagacaataaa

SEQ ID NO: 1021 - DNA, 33 bp, Artificial Sequence
  Description of Artificial Sequence Synthetic primer
tccccatttaataattccacctactatcacact

SEQ ID NO: 1022 - DNA, 33 bp, Artificial Sequence
  Description of Artificial Sequence Synthetic primer
tcccctcatgtttaaatgatcaggataaaaagc

SEQ ID NO: 1023 - DNA, 31 bp, Artificial Sequence
  Description of Artificial Sequence Synthetic primer
tcccctttaaagcaccattactcattatagt

SEQ ID NO: 1024 - DNA, 33 bp, Artificial Sequence
  Description of Artificial Sequence Synthetic primer
tcccgaacaatgagttgtatcaactatttttac

SEQ ID NO: 1025 - DNA, 21 bp, Artificial Sequence
  Description of Artificial Sequence Synthetic primer
tcccgctggcaaataaactcg

SEQ ID NO: 1026 - DNA, 25 bp, Artificial Sequence
  Description of Artificial Sequence Synthetic primer
tcccggctagagattctgtatacga

SEQ ID NO: 1027 - DNA, 28 bp, Artificial Sequence
  Description of Artificial Sequence Synthetic primer
tcccgtctgagttcctcttgcatgatca

SEQ ID NO: 1028 - DNA, 32 bp, Artificial Sequence
  Description of Artificial Sequence Synthetic primer
tccctaatagtagaaataactgcatcagtagc

SEQ ID NO: 1029 - DNA, 34 bp, Artificial Sequence
  Description of Artificial Sequence Synthetic primer
tcccttatttttctttctactaccttcggataat

SEQ ID NO: 1030 - DNA, 30 bp, Artificial Sequence
  Description of Artificial Sequence Synthetic primer
tcccttccttaatatgagaaggaaaccact

SEQ ID NO: 1031 - DNA, 32 bp, Artificial Sequence
  Description of Artificial Sequence Synthetic primer
tccgaaacttgttttgtagctttaatttgagc

SEQ ID NO: 1032 - DNA, 21 bp, Artificial Sequence
  Description of Artificial Sequence Synthetic primer
tccgaagttgccctggccgtc

SEQ ID NO: 1033 - DNA, 24 bp, Artificial Sequence
  Description of Artificial Sequence Synthetic primer
tccgagaccagcgtaggtgtaacg

SEQ ID NO: 1034 - DNA, 22 bp, Artificial Sequence
  Description of Artificial Sequence Synthetic primer
tccgataagccggattctgtgc

SEQ ID NO: 1035 - DNA, 28 bp, Artificial Sequence
  Description of Artificial Sequence Synthetic primer
tccgcaaagactttggcattaggtgtga

SEQ ID NO: 1036 - DNA, 27 bp, Artificial Sequence
  Description of Artificial Sequence Synthetic primer
tccgccaaaaactccccttttcacagg

SEQ ID NO: 1037 - DNA, 24 bp, Artificial Sequence
  Description of Artificial Sequence Synthetic primer
tccgccttcaaaatggtggcgagt

SEQ ID NO: 1038 - DNA, 31 bp, Artificial Sequence
  Description of Artificial Sequence Synthetic primer
tccggctagagattctgtatacgaaaatatc

SEQ ID NO: 1039 - DNA, 31 bp, Artificial Sequence
  Description of Artificial Sequence Synthetic primer
tccggctagagattctgtatacgacaatatc

SEQ ID NO: 1040 - DNA, 23 bp, Artificial Sequence
  Description of Artificial Sequence Synthetic primer
tccggtaactgggtcagctcgaa

SEQ ID NO: 1041 - DNA, 34 bp, Artificial Sequence
  Description of Artificial Sequence Synthetic primer
tccgtagttttgcataatttatggtctatttcaa

SEQ ID NO: 1042 - DNA, 27 bp, Artificial Sequence
  Description of Artificial Sequence Synthetic primer
tccgtcatcgctgacagaaactgagtt

SEQ ID NO: 1043 - DNA, 28 bp, Artificial Sequence
  Description of Artificial Sequence Synthetic primer
tccgtctatccacaagttaattggtact

SEQ ID NO: 1044 - DNA, 26 bp, Artificial Sequence
  Description of Artificial Sequence Synthetic primer
tcctacccaacgttcaccaagggcag

SEQ ID NO: 1045 - DNA, 29 bp, Artificial Sequence
  Description of Artificial Sequence Synthetic primer
tcctccttgtgcctcaaaacgcattttta

SEQ ID NO: 1046 - DNA, 30 bp, Artificial Sequence
  Description of Artificial Sequence Synthetic primer
tcctctatgcaacttagtatcaacaggaat

SEQ ID NO: 1047 - DNA, 24 bp, Artificial Sequence
  Description of Artificial Sequence Synthetic primer
tcctcttgggccacgcaaagtttt

SEQ ID NO: 1048 - DNA, 27 bp, Artificial Sequence
  Description of Artificial Sequence Synthetic primer
tcctcttttcacaggctctacttcatc

SEQ ID NO: 1049 - DNA, 30 bp, Artificial Sequence
  Description of Artificial Sequence Synthetic primer
tcctgaagatctagttcttgaatggttact

SEQ ID NO: 1050 - DNA, 27 bp, Artificial Sequence
  Description of Artificial Sequence Synthetic primer
tcctgcaatatctaatgcactcttacg

SEQ ID NO: 1051 - DNA, 25 bp, Artificial Sequence
  Description of Artificial Sequence Synthetic primer
tcctgcagctctacctgctccatta

SEQ ID NO: 1052 - DNA, 20 bp, Artificial Sequence
  Description of Artificial Sequence Synthetic primer
tcctggccatcctgcaggat

SEQ ID NO: 1053 - DNA, 27 bp, Artificial Sequence
  Description of Artificial Sequence Synthetic primer
tcctgttttatagccgccaagagtaag

SEQ ID NO: 1054 - DNA, 21 bp, Artificial Sequence
  Description of Artificial Sequence Synthetic primer
tccttcacgcgcatcatcacc

SEQ ID NO: 1055 - DNA, 27 bp, Artificial Sequence
  Description of Artificial Sequence Synthetic primer
tccttctgatgcctgatggaccaggag

SEQ ID NO: 1056 - DNA, 26 bp, Artificial Sequence
  Description of Artificial Sequence Synthetic primer
tccttggcatacatcatgtcgtagca

SEQ ID NO: 1057 - DNA, 33 bp, Artificial Sequence
  Description of Artificial Sequence Synthetic primer
tccttgtgcttcaaaacgcatttttacattttc

SEQ ID NO: 1058 - DNA, 28 bp, Artificial Sequence
  Description of Artificial Sequence Synthetic primer
tcctttaaaataaccgctagtagctcct

SEQ ID NO: 1059 - DNA, 30 bp, Artificial Sequence
  Description of Artificial Sequence Synthetic primer
tcctttatgcaacttagtatcaaccggaat

SEQ ID NO: 1060 - DNA, 30 bp, Artificial Sequence
  Description of Artificial Sequence Synthetic primer
tcctttatgcaacttggtatcaacaggaat

SEQ ID NO: 1061 - DNA, 30 bp, Artificial Sequence
  Description of Artificial Sequence Synthetic primer
tcctttatgcaacttggtatcaaccggaat

SEQ ID NO: 1062 - DNA, 29 bp, Artificial Sequence
  Description of Artificial Sequence Synthetic primer
tcctttcaatgttacagaaaactctacag

SEQ ID NO: 1063 - DNA, 24 bp, Artificial Sequence
  Description of Artificial Sequence Synthetic primer
tcgaaccgaagttaccctgaccat

SEQ ID NO: 1064 - DNA, 30 bp, Artificial Sequence
  Description of Artificial Sequence Synthetic primer
tcgaattcagctaaatacttttcagcatct

SEQ ID NO: 1065 - DNA, 26 bp, Artificial Sequence
  Description of Artificial Sequence Synthetic primer
tcgacctggaggacgacgtaaaatca

SEQ ID NO: 1066 - DNA, 25 bp, Artificial Sequence
  Description of Artificial Sequence Synthetic primer
tcgacgaccatcttggaaagatttc

SEQ ID NO: 1067 - DNA, 25 bp, Artificial Sequence
  Description of Artificial Sequence Synthetic primer
tcgagccgaagttaccctgtccgtc

SEQ ID NO: 1068 - DNA, 27 bp, Artificial Sequence
  Description of Artificial Sequence Synthetic primer
tcgatccgcatcaccatcaaaagcaaa

SEQ ID NO: 1069 - DNA, 26 bp, Artificial Sequence
  Description of Artificial Sequence Synthetic primer
tcgatcgaaccgaagttaccctgacc

SEQ ID NO: 1070 - DNA, 28 bp, Artificial Sequence
  Description of Artificial Sequence Synthetic primer
tcgatcgtgactctctttattttcagtt

SEQ ID NO: 1071 - DNA, 20 bp, Artificial Sequence
  Description of Artificial Sequence Synthetic primer
tcgatctccttggcgtccga

SEQ ID NO: 1072 - DNA, 26 bp, Artificial Sequence
  Description of Artificial Sequence Synthetic primer
tcgcaccgtgggttgagatgaagtac

SEQ ID NO: 1073 - DNA, 18 bp, Artificial Sequence
  Description of Artificial Sequence Synthetic primer
tcgcagcgtgcgtggcac

SEQ ID NO: 1074 - DNA, 26 bp, Artificial Sequence
  Description of Artificial Sequence Synthetic primer
tcgcaggcttacagaacgctctccta

SEQ ID NO: 1075 - DNA, 23 bp, Artificial Sequence
  Description of Artificial Sequence Synthetic primer
tcgcagttcatcagcacgaagcg

SEQ ID NO: 1076 - DNA, 26 bp, Artificial Sequence
  Description of Artificial Sequence Synthetic primer
tcgccagctagcacgatgtcattttc

SEQ ID NO: 1077 - DNA, 31 bp, Artificial Sequence
  Description of Artificial Sequence Synthetic primer
tcgccatagctaagttgtttattgtttccat

SEQ ID NO: 1078 - DNA, 22 bp, Artificial Sequence
  Description of Artificial Sequence Synthetic primer
tcgcctggtgcaggcatcatat

SEQ ID NO: 1079 - DNA, 24 bp, Artificial Sequence
  Description of Artificial Sequence Synthetic primer
tcgcgctgtatttttcctccgaga

SEQ ID NO: 1080 - DNA, 18 bp, Artificial Sequence
  Description of Artificial Sequence Synthetic primer
tcgctaccttaggaccgt

SEQ ID NO: 1081 - DNA, 29 bp, Artificial Sequence
  Description of Artificial Sequence Synthetic primer
tcgctcagcaataattcactataagccga

SEQ ID NO: 1082 - DNA, 29 bp, Artificial Sequence
  Description of Artificial Sequence Synthetic primer
tcgctctctcaagtgatctaaacttggag

SEQ ID NO: 1083 - DNA, 25 bp, Artificial Sequence
  Description of Artificial Sequence Synthetic primer
tcgcttgagtgtagtcatgattgcg

SEQ ID NO: 1084 - DNA, 32 bp, Artificial Sequence
  Description of Artificial Sequence Synthetic primer
tcggaaacaaagaattcattttctggtccaaa

SEQ ID NO: 1085 - DNA, 33 bp, Artificial Sequence
  Description of Artificial Sequence Synthetic primer
tcggaaatattctttcaatacctttatgcaact

SEQ ID NO: 1086 - DNA, 34 bp, Artificial Sequence
  Description of Artificial Sequence Synthetic primer
tcggaaatattctttcaatacctttatgcaactt

SEQ ID NO: 1087 - DNA, 34 bp, Artificial Sequence
  Description of Artificial Sequence Synthetic primer
tcggaaatattctttcaatncctttntgcaactt

SEQ ID NO: 1088 - DNA, 20 bp, Artificial Sequence
  Description of Artificial Sequence Synthetic primer
tcggactcgctttcgctacg

SEQ ID NO: 1089 - DNA, 20 bp, Artificial Sequence
  Description of Artificial Sequence Synthetic primer
tcggataagctgccacaagg

SEQ ID NO: 1090 - DNA, 19 bp, Artificial Sequence
  Description of Artificial Sequence Synthetic primer
tcggcatcacgccgtcgtc

SEQ ID NO: 1091 - DNA, 20 bp, Artificial Sequence
  Description of Artificial Sequence Synthetic primer
tcggcgaacatggccatcac

SEQ ID NO: 1092 - DNA, 33 bp, Artificial Sequence
  Description of Artificial Sequence Synthetic primer
tcgggcgtagtttttagtaattaaatcagaagt

SEQ ID NO: 1093 - DNA, 25 bp, Artificial Sequence
  Description of Artificial Sequence Synthetic primer
tcggtacgaactggatgtcgccgtt

SEQ ID NO: 1094 - DNA, 24 bp, Artificial Sequence
  Description of Artificial Sequence Synthetic primer
tcggtcagcaaaacggtagcttgc

SEQ ID NO: 1095 - DNA, 21 bp, Artificial Sequence
  Description of Artificial Sequence Synthetic primer
tcggtggtggtagccgatctc

SEQ ID NO: 1096 - DNA, 31 bp, Artificial Sequence
  Description of Artificial Sequence Synthetic primer
tcggtttaagctctacatgatcgtaaggata

SEQ ID NO: 1097 - DNA, 29 bp, Artificial Sequence
  Description of Artificial Sequence Synthetic primer
tcggtttcagtcatctccaccataaaggt

SEQ ID NO: 1098 - DNA, 26 bp, Artificial Sequence
  Description of Artificial Sequence Synthetic primer
tcgtatgaccagcttcggtactacta

SEQ ID NO: 1099 - DNA, 32 bp, Artificial Sequence
  Description of Artificial Sequence Synthetic primer
tcgtcaacactaccattattaccatgcatctc

SEQ ID NO: 1100 - DNA, 26 bp, Artificial Sequence
  Description of Artificial Sequence Synthetic primer
tcgtccgacttaacggtcagcatttc

SEQ ID NO: 1101 - DNA, 29 bp, Artificial Sequence
  Description of Artificial Sequence Synthetic primer
tcgtccgacttaacggtcagcatttcctg

SEQ ID NO: 1102 - DNA, 31 bp, Artificial Sequence
  Description of Artificial Sequence Synthetic primer
tcgtccgacttaacggtcagcatttcctgca

SEQ ID NO: 1103 - DNA, 26 bp, Artificial Sequence
  Description of Artificial Sequence Synthetic primer
tcgtcctctcgaatctccgatatacc

SEQ ID NO: 1104 - DNA, 20 bp, Artificial Sequence
  Description of Artificial Sequence Synthetic primer
tcgtcgcggacttcgaagcc

SEQ ID NO: 1105 - DNA, 26 bp, Artificial Sequence
  Description of Artificial Sequence Synthetic primer
tcgtcggacttaacggtcagcatttc

SEQ ID NO: 1106 - DNA, 29 bp, Artificial Sequence
  Description of Artificial Sequence Synthetic primer
tcgtcggacttaacggtcagcatttcctg

SEQ ID NO: 1107 - DNA, 31 bp, Artificial Sequence
  Description of Artificial Sequence Synthetic primer
tcgtcggacttaacggtcagcatttcctgca

SEQ ID NO: 1108 - DNA, 28 bp, Artificial Sequence
  Description of Artificial Sequence Synthetic primer
tcgtcgtatttatagtgaccagcaccta

SEQ ID NO: 1109 - DNA, 28 bp, Artificial Sequence
  Description of Artificial Sequence Synthetic primer
tcgtgcctaacaaatcccgtctgagttc

SEQ ID NO: 1110 - DNA, 22 bp, Artificial Sequence
  Description of Artificial Sequence Synthetic primer
tcgtggactaccagggtatcta

SEQ ID NO: 1111 - DNA, 17 bp, Artificial Sequence
  Description of Artificial Sequence Synthetic primer
tcgtgggccttgccggt

SEQ ID NO: 1112 - DNA, 28 bp, Artificial Sequence
  Description of Artificial Sequence Synthetic primer
tcgttaattaatctggctgcggaagtga

SEQ ID NO: 1113 - DNA, 27 bp, Artificial Sequence
  Description of Artificial Sequence Synthetic primer
tcgttgagatggtttttaccttcgttg

SEQ ID NO: 1114 - DNA, 25 bp, Artificial Sequence
  Description of Artificial Sequence Synthetic primer
tcgtttaagcgccagaaagcaccaa

SEQ ID NO: 1115 - DNA, 21 bp, Artificial Sequence
  Description of Artificial Sequence Synthetic primer
tcgtttcaccctgtcatgccg

SEQ ID NO: 1116 - DNA, 26 bp, Artificial Sequence
  Description of Artificial Sequence Synthetic primer
tctttcgtataaaaaggaccaattgg

SEQ ID NO: 1117 - DNA, 35 bp, Artificial Sequence
  Description of Artificial Sequence Synthetic primer
tctacaacacttgattgtaatttgccttgttcttt

SEQ ID NO: 1118 - DNA, 24 bp, Artificial Sequence
  Description of Artificial Sequence Synthetic primer
tctagcggaacaacagttctgatg

SEQ ID NO: 1119 - DNA, 27 bp, Artificial Sequence
  Description of Artificial Sequence Synthetic primer
tctatagagtccggactttcctcgtga

SEQ ID NO: 1120 - DNA, 30 bp, Artificial Sequence
  Description of Artificial Sequence Synthetic primer
tctataggtactgtagtttgttttccgtct

SEQ ID NO: 1121 - DNA, 27 bp, Artificial Sequence
  Description of Artificial Sequence Synthetic primer
tctcacctacagctttaaagccagcaa

SEQ ID NO: 1122 - DNA, 31 bp, Artificial Sequence
  Description of Artificial Sequence Synthetic primer
tctcacctacagctttaaagccagcaaaatg

SEQ ID NO: 1123 - DNA, 25 bp, Artificial Sequence
  Description of Artificial Sequence Synthetic primer
tctcatcccgatattaccgccatga

SEQ ID NO: 1124 - DNA, 29 bp, Artificial Sequence
  Description of Artificial Sequence Synthetic primer
tctcatgaaaaaggctcaggagatacaag

SEQ ID NO: 1125 - DNA, 27 bp, Artificial Sequence
  Description of Artificial Sequence Synthetic primer
tctcttaccccaccctttcacccttac

SEQ ID NO: 1126 - DNA, 31 bp, Artificial Sequence
  Description of Artificial Sequence Synthetic primer
tctctttcaaagcaccattgctcattatagt

SEQ ID NO: 1127 - DNA, 24 bp, Artificial Sequence
  Description of Artificial Sequence Synthetic primer
tctgcatttttgcgagcctgtcta

SEQ ID NO: 1128 - DNA, 28 bp, Artificial Sequence
  Description of Artificial Sequence Synthetic primer
tctgcctgagatgtcgaaaaaaacgttg

SEQ ID NO: 1129 - DNA, 26 bp, Artificial Sequence
  Description of Artificial Sequence Synthetic primer
tctggcccctccatacatgtatttag

SEQ ID NO: 1130 - DNA, 23 bp, Artificial Sequence
  Description of Artificial Sequence Synthetic primer
tctggctgcggaagtgaaatcgt

SEQ ID NO: 1131 - DNA, 23 bp, Artificial Sequence
  Description of Artificial Sequence Synthetic primer
tctgggtgacctggtgttttaga

SEQ ID NO: 1132 - DNA, 20 bp, Artificial Sequence
  Description of Artificial Sequence Synthetic primer
tctgtttcagttgcaaattc

SEQ ID NO: 1133 - DNA, 34 bp, Artificial Sequence
  Description of Artificial Sequence Synthetic primer
tcttcacacttttagaatcaaccgttttattgtc

SEQ ID NO: 1134 - DNA, 35 bp, Artificial Sequence
  Description of Artificial Sequence Synthetic primer
tcttcagcgtagtctaataatttacggaacatttc

SEQ ID NO: 1135 - DNA, 30 bp, Artificial Sequence
  Description of Artificial Sequence Synthetic primer
tcttccaaggatagatttatttcttgttcg

SEQ ID NO: 1136 - DNA, 33 bp, Artificial Sequence
  Description of Artificial Sequence Synthetic primer
tcttctgtaaagggtggtttattattcatccca

SEQ ID NO: 1137 - DNA, 32 bp, Artificial Sequence
  Description of Artificial Sequence Synthetic primer
tcttcttctttcgtataaaaaggaccaattgg

SEQ ID NO: 1138 - DNA, 28 bp, Artificial Sequence
  Description of Artificial Sequence Synthetic primer
tcttcttgaaaaattgttgtcccgaaac

SEQ ID NO: 1139 - DNA, 31 bp, Artificial Sequence
  Description of Artificial Sequence Synthetic primer
tcttctttcgtataaaaaggaccaattggtt

SEQ ID NO: 1140 - DNA, 18 bp, Artificial Sequence
  Description of Artificial Sequence Synthetic primer
tcttgacagcatccgttg

SEQ ID NO: 1141 - DNA, 32 bp, Artificial Sequence
  Description of Artificial Sequence Synthetic primer
tcttgagcattggttcttacttgttttgcata

SEQ ID NO: 1142 - DNA, 23 bp, Artificial Sequence
  Description of Artificial Sequence Synthetic primer
tcttgagccatacgtaccattgc

SEQ ID NO: 1143 - DNA, 32 bp, Artificial Sequence
  Description of Artificial Sequence Synthetic primer
tcttggcttaggatgaaaatatagtggtggta

SEQ ID NO: 1144 - DNA, 39 bp, Artificial Sequence
  Description of Artificial Sequence Synthetic primer
tctttaagttcttccaaggatagatttatttcttgttcg

SEQ ID NO: 1145 - DNA, 31 bp, Artificial Sequence
  Description of Artificial Sequence Synthetic primer
tcttttctttgcttaattttccatttgcgat

SEQ ID NO: 1146 - DNA, 14 bp, Artificial Sequence
  Description of Artificial Sequence Synthetic primer
tgttactgctggat

SEQ ID NO: 1147 - DNA, 25 bp, Artificial Sequence
  Description of Artificial Sequence Synthetic primer
tgaacatttgcgacggtatacccat

SEQ ID NO: 1148 - DNA, 31 bp, Artificial Sequence
  Description of Artificial Sequence Synthetic primer
tgaatatgtaatgcaaaccagtctttgtcat

SEQ ID NO: 1149 - DNA, 21 bp, Artificial Sequence
  Description of Artificial Sequence Synthetic primer
tgaatcttgaaacaccatacg

SEQ ID NO: 1150 - DNA, 26 bp, Artificial Sequence
  Description of Artificial Sequence Synthetic primer
tgaatcttgaaacaccatacgtaacg

SEQ ID NO: 1151 - DNA, 34 bp, Artificial Sequence
  Description of Artificial Sequence Synthetic primer
tgaattatgcaagaagtgatcaattttctcacga

SEQ ID NO: 1152 - DNA, 34 bp, Artificial Sequence
  Description of Artificial Sequence Synthetic primer
tgaattctttcaaagcaccattgctcattatagt

SEQ ID NO: 1153 - DNA, 30 bp, Artificial Sequence
  Description of Artificial Sequence Synthetic primer
tgacaggacacaatctgcatgaagtctgag

SEQ ID NO: 1154 - DNA, 25 bp, Artificial Sequence
  Description of Artificial Sequence Synthetic primer
tgacccaaagctgaaagctttactg

SEQ ID NO: 1155 - DNA, 28 bp, Artificial Sequence
  Description of Artificial Sequence Synthetic primer
tgaccccaacctggccttttgtcgttga

SEQ ID NO: 1156 - DNA, 20 bp, Artificial Sequence
  Description of Artificial Sequence Synthetic primer
tgaccgttatagttacggcc

SEQ ID NO: 1157 - DNA, 22 bp, Artificial Sequence
  Description of Artificial Sequence Synthetic primer
tgacggcatcgataccaccgtc

SEQ ID NO: 1158 - DNA, 20 bp, Artificial Sequence
  Description of Artificial Sequence Synthetic primer
tgacgtcatccccaccttcc

SEQ ID NO: 1159 - DNA, 22 bp, Artificial Sequence
  Description of Artificial Sequence Synthetic primer
tgacgtcatccccaccttcctc

SEQ ID NO: 1160 - DNA, 20 bp, Artificial Sequence
  Description of Artificial Sequence Synthetic primer
tgacgtcatgcccaccttcc

SEQ ID NO: 1161 - DNA, 20 bp, Artificial Sequence
  Description of Artificial Sequence Synthetic primer
tgacgtcatggccaccttcc

SEQ ID NO: 1162 - DNA, 32 bp, Artificial Sequence
  Description of Artificial Sequence Synthetic primer
tgacttaaacgtaccatcgcttcatatacaga

SEQ ID NO: 1163 - DNA, 27 bp, Artificial Sequence
  Description of Artificial Sequence Synthetic primer
tgactttcctcccccttatcagtctcc

SEQ ID NO: 1164 - DNA, 31 bp, Artificial Sequence
  Description of Artificial Sequence Synthetic primer
tgagatgtcgaaaaaaacgttggcaaaatac

SEQ ID NO: 1165 - DNA, 31 bp, Artificial Sequence
  Description of Artificial Sequence Synthetic primer
tgagatgttgatgatttaccagttccgattg

SEQ ID NO: 1166 - DNA, 20 bp, Artificial Sequence
  Description of Artificial Sequence Synthetic primer
tgagcatcagcgtgcgtgct

SEQ ID NO: 1167 - DNA, 28 bp, Artificial Sequence
  Description of Artificial Sequence Synthetic primer
tgagcatttttatatccatctccaccat

SEQ ID NO: 1168 - DNA, 32 bp, Artificial Sequence
  Description of Artificial Sequence Synthetic primer
tgagccatacgaacaatggtttcataaacagc

SEQ ID NO: 1169 - DNA, 32 bp, Artificial Sequence
  Description of Artificial Sequence Synthetic primer
tgagccatgagtaccatggcttcataacatgc

SEQ ID NO: 1170 - DNA, 26 bp, Artificial Sequence
  Description of Artificial Sequence Synthetic primer
tgagcgtgtggaaaaggacttggatg

SEQ ID NO: 1171 - DNA, 29 bp, Artificial Sequence
  Description of Artificial Sequence Synthetic primer
tgagctggtgctatatgaacaataccagt

SEQ ID NO: 1172 - DNA, 30 bp, Artificial Sequence
  Description of Artificial Sequence Synthetic primer
tgagtcaccctccacaatgtatagttcaga

SEQ ID NO: 1173 - DNA, 25 bp, Artificial Sequence
  Description of Artificial Sequence Synthetic primer
tgagtcgggttcactttacctggca

SEQ ID NO: 1174 - DNA, 27 bp, Artificial Sequence
  Description of Artificial Sequence Synthetic primer
tgagtctacacttggcttaggatgaaa

SEQ ID NO: 1175 - DNA, 32 bp, Artificial Sequence
  Description of Artificial Sequence Synthetic primer
tgagttaaaatgcgattgatttcagtttccaa

SEQ ID NO: 1176 - DNA, 32 bp, Artificial Sequence
  Description of Artificial Sequence Synthetic primer
tgagtttgaaccatttcagagcgaatatctac

SEQ ID NO: 1177 - DNA, 28 bp, Artificial Sequence
  Description of Artificial Sequence Synthetic primer
tgagtttgcacttcaaaagaaattgtgt

SEQ ID NO: 1178 - DNA, 29 bp, Artificial Sequence
  Description of Artificial Sequence Synthetic primer
tgataaaaagcactaagcgatgaaacagc

SEQ ID NO: 1179 - DNA, 27 bp, Artificial Sequence
  Description of Artificial Sequence Synthetic primer
tgataatgaagggaaacctttttcacg

SEQ ID NO: 1180 - DNA, 32 bp, Artificial Sequence
  Description of Artificial Sequence Synthetic primer
tgatattgaactggtgtaccataatagttgcc

SEQ ID NO: 1181 - DNA, 30 bp, Artificial Sequence
  Description of Artificial Sequence Synthetic primer
tgatcctgaatgtttatatctttaacgcct

SEQ ID NO: 1182 - DNA, 22 bp, Artificial Sequence
  Description of Artificial Sequence Synthetic primer
tgatctccatggcgcggatctt

SEQ ID NO: 1183 - DNA, 19 bp, Artificial Sequence
  Description of Artificial Sequence Synthetic primer
tgatgcgggctggttcaac

SEQ ID NO: 1184 - DNA, 24 bp, Artificial Sequence
  Description of Artificial Sequence Synthetic primer
tgatgcgggctggttcaacaagag

SEQ ID NO: 1185 - DNA, 30 bp, Artificial Sequence
  Description of Artificial Sequence Synthetic primer
tgatggtctatttcaatggcagttacgaaa

SEQ ID NO: 1186 - DNA, 19 bp, Artificial Sequence
  Description of Artificial Sequence Synthetic primer
tgattatcagcggaagtag

SEQ ID NO: 1187 - DNA, 28 bp, Artificial Sequence
  Description of Artificial Sequence Synthetic primer
tgattcaaatgcagaaccatcaaactcg

SEQ ID NO: 1188 - DNA, 31 bp, Artificial Sequence
  Description of Artificial Sequence Synthetic primer
tgattcgatcatacgagacattaaaactgag

SEQ ID NO: 1189 - DNA, 30 bp, Artificial Sequence
  Description of Artificial Sequence Synthetic primer
tgattggcgataaagtgatattttctaaaa

SEQ ID NO: 1190 - DNA, 22 bp, Artificial Sequence
  Description of Artificial Sequence Synthetic primer
tgattgttttgcagctgattgt

SEQ ID NO: 1191 - DNA, 22 bp, Artificial Sequence
  Description of Artificial Sequence Synthetic primer
tgattgttttgcagctgattgt

SEQ ID NO: 1192 - DNA, 30 bp, Artificial Sequence
  Description of Artificial Sequence Synthetic primer
tgcaaaagtaacggttacatctgctccaat

SEQ ID NO: 1193 - DNA, 33 bp, Artificial Sequence
  Description of Artificial Sequence Synthetic primer
tgcaacaattaatgctccgacaattaaaggatt

SEQ ID NO: 1194 - DNA, 24 bp, Artificial Sequence
  Description of Artificial Sequence Synthetic primer
tgcaactcatctggtttaggatct

SEQ ID NO: 1195 - DNA, 32 bp, Artificial Sequence
  Description of Artificial Sequence Synthetic primer
tgcaactgaatagattgcagtaagttataagc

SEQ ID NO: 1196 - DNA, 23 bp, Artificial Sequence
  Description of Artificial Sequence Synthetic primer
tgcaagagcaaccctagtgttcg

SEQ ID NO: 1197 - DNA, 29 bp, Artificial Sequence
  Description of Artificial Sequence Synthetic primer
tgcaagggaaacctagaattacaaaccct

SEQ ID NO: 1198 - DNA, 29 bp, Artificial Sequence
  Description of Artificial Sequence Synthetic primer
tgcaatgtgtgctatgtcagcaaaaagat

SEQ ID NO: 1199 - DNA, 18 bp, Artificial Sequence
  Description of Artificial Sequence Synthetic primer
tgcacctgcggtcgagcg

SEQ ID NO: 1200 - DNA, 24 bp, Artificial Sequence
  Description of Artificial Sequence Synthetic primer
tgcacgcaaacgctttacttcagc

SEQ ID NO: 1201 - DNA, 26 bp, Artificial Sequence
  Description of Artificial Sequence Synthetic primer
tgcacgtctgtttcagttgcaaattc

SEQ ID NO: 1202 - DNA, 13 bp, Artificial Sequence
  Description of Artificial Sequence Synthetic primer
tgcagctgattgt

SEQ ID NO: 1203 - DNA, 26 bp, Artificial Sequence
  Description of Artificial Sequence Synthetic primer
tgcatagggaaggtaacaccatagtt

SEQ ID NO: 1204 - DNA, 25 bp, Artificial Sequence
  Description of Artificial Sequence Synthetic primer
tgcatcaccatttccttgtccttcg

SEQ ID NO: 1205 - DNA, 35 bp, Artificial Sequence
  Description of Artificial Sequence Synthetic primer
tgcatgaagcataaaaactgtatcaagtgctttta

SEQ ID NO: 1206 - DNA, 36 bp, Artificial Sequence
  Description of Artificial Sequence Synthetic primer
tgcatgcttactcaaatcatcataaacaattaaagc

SEQ ID NO: 1207 - DNA, 29 bp, Artificial Sequence
  Description of Artificial Sequence Synthetic primer
tgcattgtaccgaagtagttcacattgtt

SEQ ID NO: 1208 - DNA, 25 bp, Artificial Sequence
  Description of Artificial Sequence Synthetic primer
tgccaagtgctggtttaccccatgg

SEQ ID NO: 1209 - DNA, 26 bp, Artificial Sequence
  Description of Artificial Sequence Synthetic primer
tgccactttgacaactcctgttgctg

SEQ ID NO: 1210 - DNA, 21 bp, Artificial Sequence
  Description of Artificial Sequence Synthetic primer
tgccagcgacagaccatcgta

SEQ ID NO: 1211 - DNA, 24 bp, Artificial Sequence
  Description of Artificial Sequence Synthetic primer
tgccagcttagtcatacggacttc

SEQ ID NO: 1212 - DNA, 27 bp, Artificial Sequence
  Description of Artificial Sequence Synthetic primer
tgccagtttccacatttcacgttcgtg

SEQ ID NO: 1213 - DNA, 30 bp, Artificial Sequence
  Description of Artificial Sequence Synthetic primer
tgccatacgtaccatcgtttcataaacagc

SEQ ID NO: 1214 - DNA, 24 bp, Artificial Sequence
  Description of Artificial Sequence Synthetic primer
tgccatagcaaagcctacagcatt

SEQ ID NO: 1215 - DNA, 28 bp, Artificial Sequence
  Description of Artificial Sequence Synthetic primer
tgccatccataatcacgccatactgacg

SEQ ID NO: 1216 - DNA, 30 bp, Artificial Sequence
  Description of Artificial Sequence Synthetic primer
tgccatttccatgtactcttctctaacatt

SEQ ID NO: 1217 - DNA, 27 bp, Artificial Sequence
  Description of Artificial Sequence Synthetic primer
tgcccaccagaaagactagcaggataa

SEQ ID NO: 1218 - DNA, 20 bp, Artificial Sequence
  Description of Artificial Sequence Synthetic primer
tgcccaggtacaacctgcat

SEQ ID NO: 1219 - DNA, 27 bp, Artificial Sequence
  Description of Artificial Sequence Synthetic primer
tgccccattgctcatgatagtagctac

SEQ ID NO: 1220 - DNA, 30 bp, Artificial Sequence
  Description of Artificial Sequence Synthetic primer
tgccctttctaaaagtcttgagtgaagata

SEQ ID NO: 1221 - DNA, 25 bp, Artificial Sequence
  Description of Artificial Sequence Synthetic primer
tgcccttttgtaaaagcagggctat

SEQ ID NO: 1222 - DNA, 23 bp, Artificial Sequence
  Description of Artificial Sequence Synthetic primer
tgccgataagccggattctgtgc

SEQ ID NO: 1223 - DNA, 28 bp, Artificial Sequence
  Description of Artificial Sequence Synthetic primer
tgccgtaacatagaagttaccgttgatt

SEQ ID NO: 1224 - DNA, 33 bp, Artificial Sequence
  Description of Artificial Sequence Synthetic primer
tgccgtaactaacataagagaattatgcaagaa

SEQ ID NO: 1225 - DNA, 33 bp, Artificial Sequence
  Description of Artificial Sequence Synthetic primer
tgccgtatacgaaaatatcttatcatttagcgt

SEQ ID NO: 1226 - DNA, 25 bp, Artificial Sequence
  Description of Artificial Sequence Synthetic primer
tgcctaacaaatcccgtctgagttc

SEQ ID NO: 1227 - DNA, 20 bp, Artificial Sequence
  Description of Artificial Sequence Synthetic primer
tgcctcgcgcaacctacccg

SEQ ID NO: 1228 - DNA, 20 bp, Artificial Sequence
  Description of Artificial Sequence Synthetic primer
tgcctcgtgcaacccacccg

SEQ ID NO: 1229 - DNA, 22 bp, Artificial Sequence
  Description of Artificial Sequence Synthetic primer
tgcgaggaacttcacgtcctgc

SEQ ID NO: 1230 - DNA, 30 bp, Artificial Sequence
  Description of Artificial Sequence Synthetic primer
tgcgatggtaggtatcttagcaatcattct

SEQ ID NO: 1231 - DNA, 24 bp, Artificial Sequence
  Description of Artificial Sequence Synthetic primer
tgcgcgagcttttatttgggtttc

SEQ ID NO: 1232 - DNA, 29 bp, Artificial Sequence
  Description of Artificial Sequence Synthetic primer
tgcgctaattcttcaacttcttctttcgt

SEQ ID NO: 1233 - DNA, 32 bp, Artificial Sequence
  Description of Artificial Sequence Synthetic primer
tgcgctatcaacgattttgacaatatatgtga

SEQ ID NO: 1234 - DNA, 23 bp, Artificial Sequence
  Description of Artificial Sequence Synthetic primer
tgcggcagcactatcaccatcca

SEQ ID NO: 1235 - DNA, 21 bp, Artificial Sequence
  Description of Artificial Sequence Synthetic primer
tgcgggctggttcaacaagag

SEQ ID NO: 1236 - DNA, 23 bp, Artificial Sequence
  Description of Artificial Sequence Synthetic primer
tgcgggtgatacttaccgagtac

SEQ ID NO: 1237 - DNA, 22 bp, Artificial Sequence
  Description of Artificial Sequence Synthetic primer
tgcggtctggcgcatataggta

SEQ ID NO: 1238 - DNA, 30 bp, Artificial Sequence
  Description of Artificial Sequence Synthetic primer
tgcgtagtctaataatttacggaacatttc

SEQ ID NO: 1239 - DNA, 29 bp, Artificial Sequence
  Description of Artificial Sequence Synthetic primer
tgcgtgacgaccttcttgaattgtaatca

SEQ ID NO: 1240 - DNA, 23 bp, Artificial Sequence
  Description of Artificial Sequence Synthetic primer
tgcgtggactaccagggtatcta

SEQ ID NO: 1241 - DNA, 13 bp, Artificial Sequence
  Description of Artificial Sequence Synthetic primer
tgcagctgattgt

SEQ ID NO: 1242 - DNA, 30 bp, Artificial Sequence
  Description of Artificial Sequence Synthetic primer
tgctaaagtcttgagccatacgaacaatgg

SEQ ID NO: 1243 - DNA, 24 bp, Artificial Sequence
  Description of Artificial Sequence Synthetic primer
tgctagacctttacgtgcaccgtg

SEQ ID NO: 1244 - DNA, 23 bp, Artificial Sequence
  Description of Artificial Sequence Synthetic primer
tgctaggccatcaggccacgcat

SEQ ID NO: 1245 - DNA, 34 bp, Artificial Sequence
  Description of Artificial Sequence Synthetic primer
tgctatatgctacaactggttcaaaaacattaag

SEQ ID NO: 1246 - DNA, 29 bp, Artificial Sequence
  Description of Artificial Sequence Synthetic primer
tgctcacctgctacaacaagtccagcaat

SEQ ID NO: 1247 - DNA, 29 bp, Artificial Sequence
  Description of Artificial Sequence Synthetic primer
tgctcttacctcaccgttccacccttacc

SEQ ID NO: 1248 - DNA, 29 bp, Artificial Sequence
  Description of Artificial Sequence Synthetic primer
tgctgctttcgcatggttaattgcttcaa

SEQ ID NO: 1249 - DNA, 27 bp, Artificial Sequence
  Description of Artificial Sequence Synthetic primer
tgctgctttgatggctgaatccccttc

SEQ ID NO: 1250 - DNA, 22 bp, Artificial Sequence
  Description of Artificial Sequence Synthetic primer
tgctggattcgcctttgctacg

SEQ ID NO: 1251 - DNA, 21 bp, Artificial Sequence
  Description of Artificial Sequence Synthetic primer
tgctgtagggaaatcagggcc

SEQ ID NO: 1252 - DNA, 18 bp, Artificial Sequence
  Description of Artificial Sequence Synthetic primer
tgcttagatgctttcagc

SEQ ID NO: 1253 - DNA, 34 bp, Artificial Sequence
  Description of Artificial Sequence Synthetic primer
tgcttcaaaacgcatttttacattttcgttaaag

SEQ ID NO: 1254 - DNA, 27 bp, Artificial Sequence
  Description of Artificial Sequence Synthetic primer
tgcttcagcacggccaccaacttctag

SEQ ID NO: 1255 - DNA, 31 bp, Artificial Sequence
  Description of Artificial Sequence Synthetic primer
tgcttcagcgtagtctaataatttacggaac

SEQ ID NO: 1256 - DNA, 20 bp, Artificial Sequence
  Description of Artificial Sequence Synthetic primer
tgcttctcttccgggtcggc

SEQ ID NO: 1257 - DNA, 32 bp, Artificial Sequence
  Description of Artificial Sequence Synthetic primer
tgcttgctcaaatcatcataaacaattaaagc

SEQ ID NO: 1258 - DNA, 31 bp, Artificial Sequence
  Description of Artificial Sequence Synthetic primer
tgcttgctctttcaagcagtcttgaatgaag

SEQ ID NO: 1259 - DNA, 23 bp, Artificial Sequence
  Description of Artificial Sequence Synthetic primer
tgcttggtggcttcttcgtcgaa

SEQ ID NO: 1260 - DNA, 32 bp, Artificial Sequence
  Description of Artificial Sequence Synthetic primer
tgctttgtaatctagttcctgaatagtaacca

SEQ ID NO: 1261 - DNA, 30 bp, Artificial Sequence
  Description of Artificial Sequence Synthetic primer
tggaaaactcatgaaattaaagtgaaagga

SEQ ID NO: 1262 - DNA, 29 bp, Artificial Sequence
  Description of Artificial Sequence Synthetic primer
tggaaaccggctaagtgagtaccaccatc

SEQ ID NO: 1263 - DNA, 30 bp, Artificial Sequence
  Description of Artificial Sequence Synthetic primer
tggaacaccgtctttaattaaagtatctcc

SEQ ID NO: 1264 - DNA, 24 bp, Artificial Sequence
  Description of Artificial Sequence Synthetic primer
tggaatttaccagcgatagacacc

SEQ ID NO: 1265 - DNA, 20 bp, Artificial Sequence
  Description of Artificial Sequence Synthetic primer
tggaccacgccgaagaacgg

SEQ ID NO: 1266 - DNA, 30 bp, Artificial Sequence
  Description of Artificial Sequence Synthetic primer
tggacgatattcacggtttacccacttata

SEQ ID NO: 1267 - DNA, 31 bp, Artificial Sequence
  Description of Artificial Sequence Synthetic primer
tggactaataacaatgagctcattgtactga

SEQ ID NO: 1268 - DNA, 31 bp, Artificial Sequence
  Description of Artificial Sequence Synthetic primer
tggataattggtcgtaacaagggatagtgag

SEQ ID NO: 1269 - DNA, 27 bp, Artificial Sequence
  Description of Artificial Sequence Synthetic primer
tggatagacgtcatatgaaggtgtgct

SEQ ID NO: 1270 - DNA, 27 bp, Artificial Sequence
  Description of Artificial Sequence Synthetic primer
tggatcactgcttacgaactcagcttc

SEQ ID NO: 1271 - DNA, 26 bp, Artificial Sequence
  Description of Artificial Sequence Synthetic primer
tggatgtgctcacgagtctgtggcat

SEQ ID NO: 1272 - DNA, 23 bp, Artificial Sequence
  Description of Artificial Sequence Synthetic primer
tggcaacagctcaacacctttgg

SEQ ID NO: 1273 - DNA, 26 bp, Artificial Sequence
  Description of Artificial Sequence Synthetic primer
tggcaccgtgggttgagatgaagtac

SEQ ID NO: 1274 - DNA, 19 bp, Artificial Sequence
  Description of Artificial Sequence Synthetic primer
tggcacgagcctgacctgt

SEQ ID NO: 1275 - DNA, 34 bp, Artificial Sequence
  Description of Artificial Sequence Synthetic primer
tggcagcaatagtttgacgtacaaatgcacacat

SEQ ID NO: 1276 - DNA, 26 bp, Artificial Sequence
  Description of Artificial Sequence Synthetic primer
tggcatcaccatttccttgtccttcg

SEQ ID NO: 1277 - DNA, 29 bp, Artificial Sequence
  Description of Artificial Sequence Synthetic primer
tggccacttttatcagcaaccttacagtc

SEQ ID NO: 1278 - DNA, 19 bp, Artificial Sequence
  Description of Artificial Sequence Synthetic primer
tggccgtactccccaggcg

SEQ ID NO: 1279 - DNA, 20 bp, Artificial Sequence
  Description of Artificial Sequence Synthetic primer
tggcgatgcactggcttgag

SEQ ID NO: 1280 - DNA, 24 bp, Artificial Sequence
  Description of Artificial Sequence Synthetic primer
tggctcataagacgcgcttgtaga

SEQ ID NO: 1281 - DNA, 22 bp, Artificial Sequence
  Description of Artificial Sequence Synthetic primer
tggctgcggaagtgaaatcgta

SEQ ID NO: 1282 - DNA, 19 bp, Artificial Sequence
  Description of Artificial Sequence Synthetic primer
tggctgcttctaagccaac

SEQ ID NO: 1283 - DNA, 26 bp, Artificial Sequence
  Description of Artificial Sequence Synthetic primer
tggcttgagaatttaggatccggcac

SEQ ID NO: 1284 - DNA, 30 bp, Artificial Sequence
  Description of Artificial Sequence Synthetic primer
tgggacgtaatcgtataaattcatcatttc

SEQ ID NO: 1285 - DNA, 36 bp, Artificial Sequence
  Description of Artificial Sequence Synthetic primer
tgggataacattggttggaatataagcagaaacatc

SEQ ID NO: 1286 - DNA, 30 bp, Artificial Sequence
  Description of Artificial Sequence Synthetic primer
tgggatggaggtgtagaaggtgttatcatc

SEQ ID NO: 1287 - DNA, 30 bp, Artificial Sequence; identical to SEQ ID NO: 1286
  Description of Artificial Sequence Synthetic primer

SEQ ID NO: 1288 - DNA, 32 bp, Artificial Sequence
  Description of Artificial Sequence Synthetic primer
tgggcaccatttatccacaaattgattggtat

SEQ ID NO: 1289 - DNA, 31 bp, Artificial Sequence
  Description of Artificial Sequence Synthetic primer
tggggacttccttaccacttttagtatctaa

SEQ ID NO: 1290 - DNA, 33 bp, Artificial Sequence
  Description of Artificial Sequence Synthetic primer
tggggatatggaggtgtagaaggtgttatcatc

SEQ ID NO: 1291 - DNA, 27 bp, Artificial Sequence
  Description of Artificial Sequence Synthetic primer
tggggtaagacgcggctagcatgtatt

SEQ ID NO: 1292 - DNA, 25 bp, Artificial Sequence
  Description of Artificial Sequence Synthetic primer
tgggtacgaactggatgtcgccgtt

SEQ ID NO: 1293 - DNA, 27 bp, Artificial Sequence
  Description of Artificial Sequence Synthetic primer
tgggtaggtttttatctgtgacgcctt

SEQ ID NO: 1294 - DNA, 26 bp, Artificial Sequence
  Description of Artificial Sequence Synthetic primer
tgggtctacacctgcacttgcataac

SEQ ID NO: 1295 - DNA, 24 bp, Artificial Sequence
  Description of Artificial Sequence Synthetic primer
tgggtgctggtttaccccatggag

SEQ ID NO: 1296 - DNA, 29 bp, Artificial Sequence
  Description of Artificial Sequence Synthetic primer
tgggttgcgttgcagattatctttaccaa

SEQ ID NO: 1297 - DNA, 25 bp, Artificial Sequence
  Description of Artificial Sequence Synthetic primer
tgggtttcgcgcttagatgctttca

SEQ ID NO: 1298 - DNA, 18 bp, Artificial Sequence
  Description of Artificial Sequence Synthetic primer
tggtaacccttgtctttg

SEQ ID NO: 1299 - DNA, 31 bp, Artificial Sequence
  Description of Artificial Sequence Synthetic primer
tggtaacccttgtctttgaattgtatttgca

SEQ ID NO: 1300 - DNA, 33 bp, Artificial Sequence
  Description of Artificial Sequence Synthetic primer
tggtacaacatcgttagctttaccactttcacg

SEQ ID NO: 1301 - DNA, 32 bp, Artificial Sequence
  Description of Artificial Sequence Synthetic primer
tggtacacctggtttcgttttgatgatttgta

SEQ ID NO: 1302 - DNA, 30 bp, Artificial Sequence
  Description of Artificial Sequence Synthetic primer
tggtacttcaacttcatccattatgaagtc

SEQ ID NO: 1303 - DNA, 31 bp, Artificial Sequence
  Description of Artificial Sequence Synthetic primer
tggtatattcgttaattaatctggctgcgga

SEQ ID NO: 1304 - DNA, 21 bp, Artificial Sequence
  Description of Artificial Sequence Synthetic primer
tggtctgagtacctcctttgc

SEQ ID NO: 1305 - DNA, 31 bp, Artificial Sequence
  Description of Artificial Sequence Synthetic primer
tggtgggtatcttagcaatcattctaatagc

SEQ ID NO: 1306 - DNA, 32 bp, Artificial Sequence
  Description of Artificial Sequence Synthetic primer
tggtgttctagtatagattgaggtagtggtga

SEQ ID NO: 1307 - DNA, 24 bp, Artificial Sequence
  Description of Artificial Sequence Synthetic primer
tggttagaagtcgtaacgtggacc

SEQ ID NO: 1308 - DNA, 25 bp, Artificial Sequence
  Description of Artificial Sequence Synthetic primer
tggttcaacaagagttgccgttgca

SEQ ID NO: 1309 - DNA, 31 bp, Artificial Sequence
  Description of Artificial Sequence Synthetic primer
tggttcttacttgctttgcataaactttcca

SEQ ID NO: 1310 - DNA, 31 bp, Artificial Sequence
  Description of Artificial Sequence Synthetic primer
tggttgtagttcctgtagttgttgcattaac

SEQ ID NO: 1311 - DNA, 30 bp, Artificial Sequence
  Description of Artificial Sequence Synthetic primer
tggtttgtcagaatcacgttctggagttgg

SEQ ID NO: 1312 - DNA, 28 bp, Artificial Sequence
  Description of Artificial Sequence Synthetic primer
tgtaaaagcagggctataataaggactc

SEQ ID NO: 1313 - DNA, 29 bp, Artificial Sequence
  Description of Artificial Sequence Synthetic primer
tgtaaattccgcaaagactttggcattag

SEQ ID NO: 1314 - DNA, 29 bp, Artificial Sequence
  Description of Artificial Sequence Synthetic primer
tgtaacccttgtctttgaattgtatttgc

SEQ ID NO: 1315 - DNA, 31 bp, Artificial Sequence
  Description of Artificial Sequence Synthetic primer
tgtaattaaccgaaggttctgtagaagtatg

SEQ ID NO: 1316 - DNA, 28 bp, Artificial Sequence
  Description of Artificial Sequence Synthetic primer
tgtacaaggaccattataatcaatgcca

SEQ ID NO: 1317 - DNA, 31 bp, Artificial Sequence
  Description of Artificial Sequence Synthetic primer
tgtacaataaggagtcaccttatgtccctta

SEQ ID NO: 1318 - DNA, 30 bp, Artificial Sequence
  Description of Artificial Sequence Synthetic primer
tgtacaccatttatccacaaattgattggt

SEQ ID NO: 1319 - DNA, 29 bp, Artificial Sequence
  Description of Artificial Sequence Synthetic primer
tgtaggcaagtgcataagaaattgataca

SEQ ID NO: 1320 - DNA, 27 bp, Artificial Sequence
  Description of Artificial Sequence Synthetic primer
tgtcaatatgaaggtgctctgtggata

SEQ ID NO: 1321 - DNA, 28 bp, Artificial Sequence
  Description of Artificial Sequence Synthetic primer
tgtcaccagcttcagcgtagtctaataa

SEQ ID NO: 1322 - DNA, 19 bp, Artificial Sequence
  Description of Artificial Sequence Synthetic primer
tgtcactcccgacacgcca

SEQ ID NO: 1323 - DNA, 29 bp, Artificial Sequence
  Description of Artificial Sequence Synthetic primer
tgtcagctaagctaataacgtttgtagag

SEQ ID NO: 1324 - DNA, 26 bp, Artificial Sequence
  Description of Artificial Sequence Synthetic primer
tgtcatcaagcaccccaaaatgaact

SEQ ID NO: 1325 - DNA, 28 bp, Artificial Sequence
  Description of Artificial Sequence Synthetic primer
tgtccgacttgacggtcaacatttcctg

SEQ ID NO: 1326 - DNA, 28 bp, Artificial Sequence
  Description of Artificial Sequence Synthetic primer
tgtccgacttgacggtcagcatttcctg

SEQ ID NO: 1327 - DNA, 28 bp, Artificial Sequence
  Description of Artificial Sequence Synthetic primer
tgtccgacttgacggttagcatttcctg

SEQ ID NO: 1328 - DNA, 21 bp, Artificial Sequence
  Description of Artificial Sequence Synthetic primer
tgtcgcagcatctgttcctgc

SEQ ID NO: 1329 - DNA, 29 bp, Artificial Sequence
  Description of Artificial Sequence Synthetic primer
tgtctattgtcgattgttacctgtacagt

SEQ ID NO: 1330 - DNA, 27 bp, Artificial Sequence
  Description of Artificial Sequence Synthetic primer
tgtgaacatttgcgacggtatacccat

SEQ ID NO: 1331 - DNA, 29 bp, Artificial Sequence
  Description of Artificial Sequence Synthetic primer
tgtgaagaactttcaaatctgtgaatcca

SEQ ID NO: 1332 - DNA, 24 bp, Artificial Sequence
  Description of Artificial Sequence Synthetic primer
tgtgatatggaggtgtagaaggtg

SEQ ID NO: 1333 - DNA, 27 bp, Artificial Sequence
  Description of Artificial Sequence Synthetic primer
tgtgatatggaggtgtagaaggtgtta

SEQ ID NO: 1334 - DNA, 24 bp, Artificial Sequence
  Description of Artificial Sequence Synthetic primer
tgtgcaggcatcatgtcataccaa

SEQ ID NO: 1335 - DNA, 31 bp, Artificial Sequence
  Description of Artificial Sequence Synthetic primer
tgtgctgctttcgcatggttaattgcttcaa

SEQ ID NO: 1336 - DNA, 22 bp, Artificial Sequence
  Description of Artificial Sequence Synthetic primer
tgtgctggtttaccccatggag

SEQ ID NO: 1337 - DNA, 23 bp, Artificial Sequence
  Description of Artificial Sequence Synthetic primer
tgtgctggtttaccccatggagt

SEQ ID NO: 1338 - DNA, 15 bp, Artificial Sequence
  Description of Artificial Sequence Synthetic primer
tgtgctttgaatgct

SEQ ID NO: 1339 - DNA, 28 bp, Artificial Sequence
  Description of Artificial Sequence Synthetic primer
tgtgctttttttgctgccatagcaaagc

SEQ ID NO: 1340 - DNA, 26 bp, Artificial Sequence
  Description of Artificial Sequence Synthetic primer
tgtggccgatttcaccacctgctcct

SEQ ID NO: 1341 - DNA, 18 bp, Artificial Sequence
  Description of Artificial Sequence Synthetic primer
tgtgttgtcgccgcgcag

SEQ ID NO: 1342 - DNA, 20 bp, Artificial Sequence
  Description of Artificial Sequence Synthetic primer
tgttaacggcttcaagaccc

SEQ ID NO: 1343 - DNA, 28 bp, Artificial Sequence
  Description of Artificial Sequence Synthetic primer
tgttaagtgtgttgcggctgtctttatt

SEQ ID NO: 1344 - DNA, 36 bp, Artificial Sequence
  Description of Artificial Sequence Synthetic primer
tgttaatggtaacccttgtctttgaattgtatttgc

SEQ ID NO: 1345 - DNA, 22 bp, Artificial Sequence
  Description of Artificial Sequence Synthetic primer
tgttactcacccgtctgccact

SEQ ID NO: 1346 - DNA, 14 bp, Artificial Sequence
  Description of Artificial Sequence Synthetic primer
tgttactgctggat

SEQ ID NO: 1347 - DNA, 34 bp, Artificial Sequence
  Description of Artificial Sequence Synthetic primer
tgttcatgtttaaatgatcaggataaaaagcact

SEQ ID NO: 1348 - DNA, 30 bp, Artificial Sequence
  Description of Artificial Sequence Synthetic primer
tgttccaatagcagttccgcccaaattgat

SEQ ID NO: 1349 - DNA, 30 bp, Artificial Sequence
  Description of Artificial Sequence Synthetic primer
tgttctggattgattgcacaatcaccaaag

SEQ ID NO: 1350 - DNA, 30 bp, Artificial Sequence
  Description of Artificial Sequence Synthetic primer
tgttcttgatacacctggtttcgttttgat

SEQ ID NO: 1351 - DNA, 30 bp, Artificial Sequence
  Description of Artificial Sequence Synthetic primer
tgttgaagctgtacttgacctgattttacg

SEQ ID NO: 1352 - DNA, 19 bp, Artificial Sequence
  Description of Artificial Sequence Synthetic primer
tgttgaccatgcttcttag

SEQ ID NO: 1353 - DNA, 28 bp, Artificial Sequence
  Description of Artificial Sequence Synthetic primer
tgttgtgccgcagtcaaatatctaaata

SEQ ID NO: 1354 - DNA, 25 bp, Artificial Sequence
  Description of Artificial Sequence Synthetic primer
tgtttgtgatgcatttgctgagcta

SEQ ID NO: 1355 - DNA, 33 bp, Artificial Sequence
  Description of Artificial Sequence Synthetic primer
tgttttatgtgtagttgagcttactacatgagc

SEQ ID NO: 1356 - DNA, 29 bp, Artificial Sequence
  Description of Artificial Sequence Synthetic primer
tgttttgtatccaagtgctggtttacccc

SEQ ID NO: 1357 - DNA, 11 bp, Artificial Sequence
  Description of Artificial Sequence Synthetic primer
tactcatgcca

SEQ ID NO: 1358 - DNA, 11 bp, Artificial Sequence
  Description of Artificial Sequence Synthetic primer
tattcttcgtt

SEQ ID NO: 1359 - DNA, 17 bp, Artificial Sequence
  Description of Artificial Sequence Synthetic primer
ttacttctaacccactc

SEQ ID NO: 1360 - DNA, 27 bp, Artificial Sequence
  Description of Artificial Sequence Synthetic primer
ttaatctggctgcggaagtgaaatcgt

SEQ ID NO: 1361 - DNA, 28 bp, Artificial Sequence
  Description of Artificial Sequence Synthetic primer
ttaccatcttcaaatacccgaacagtaa

SEQ ID NO: 1362 - DNA, 26 bp, Artificial Sequence
  Description of Artificial Sequence Synthetic primer
ttaccgagcaggttctgacggaaacg

SEQ ID NO: 1363 - DNA, 20 bp, Artificial Sequence
  Description of Artificial Sequence Synthetic primer
ttacgccatcaggccacgca

SEQ ID NO: 1364 - DNA, 17 bp, Artificial Sequence
  Description of Artificial Sequence Synthetic primer
ttactcacccgtccgcc

SEQ ID NO: 1365 - DNA, 20 bp, Artificial Sequence
  Description of Artificial Sequence Synthetic primer
ttactcacccgtccgccgct

SEQ ID NO: 1366 - DNA, 34 bp, Artificial Sequence
  Description of Artificial Sequence Synthetic primer
ttacttccttaccacttttagtatctaaagcata

SEQ ID NO: 1367 - DNA, 17 bp, Artificial Sequence
  Description of Artificial Sequence Synthetic primer
ttacttctaacccactc

SEQ ID NO: 1368 - DNA, 21 bp, Artificial Sequence
  Description of Artificial Sequence Synthetic primer
ttagaagtcgtaacgtggacc

SEQ ID NO: 1369 - DNA, 22 bp, Artificial Sequence
  Description of Artificial Sequence Synthetic primer
ttagatgctttcagcacttatc

SEQ ID NO: 1370 - DNA, 26 bp, Artificial Sequence
  Description of Artificial Sequence Synthetic primer
ttcaaaaccttgctctcgccaaacaa

SEQ ID NO: 1371 - DNA, 24 bp, Artificial Sequence
  Description of Artificial Sequence Synthetic primer
ttcaaaagttgctcgagaccattg

SEQ ID NO: 1372 - DNA, 23 bp, Artificial Sequence
  Description of Artificial Sequence Synthetic primer
ttcaaaatgcggaggcgtatgtg

SEQ ID NO: 1373 - DNA, 22 bp, Artificial Sequence
  Description of Artificial Sequence Synthetic primer
ttcaacaagagttgccgttgca

SEQ ID NO: 1374 - DNA, 28 bp, Artificial Sequence
  Description of Artificial Sequence Synthetic primer
ttcaacactctcacctacagctttaaag

SEQ ID NO: 1375 - DNA, 24 bp, Artificial Sequence
  Description of Artificial Sequence Synthetic primer
ttcaagtgcttgctcaccattgtc

SEQ ID NO: 1376 - DNA, 26 bp, Artificial Sequence
  Description of Artificial Sequence Synthetic primer
ttcaggtacagcaggtggttcaggat

SEQ ID NO: 1377 - DNA, 23 bp, Artificial Sequence
  Description of Artificial Sequence Synthetic primer
ttcaggtccatcgggttcatgcc

SEQ ID NO: 1378 - DNA, 29 bp, Artificial Sequence
  Description of Artificial Sequence Synthetic primer
ttcataagcaatacctttacttgcaccac

SEQ ID NO: 1379 - DNA, 30 bp, Artificial Sequence
  Description of Artificial Sequence Synthetic primer
ttcattttctggtccaaagtaagcagtatc

SEQ ID NO: 1380 - DNA, 25 bp, Artificial Sequence
  Description of Artificial Sequence Synthetic primer
ttccaagtgctggtttaccccatgg

SEQ ID NO: 1381 - DNA, 33 bp, Artificial Sequence
  Description of Artificial Sequence Synthetic primer
ttccaccttggatacctggaaaaatagctgaat

SEQ ID NO: 1382 - DNA, 36 bp, Artificial Sequence
  Description of Artificial Sequence Synthetic primer
ttccatttcaactaattctaataattcttcatcgtc

SEQ ID NO: 1383 - DNA, 28 bp, Artificial Sequence
  Description of Artificial Sequence Synthetic primer
ttcccctgaccttcgattaaaggatagc

SEQ ID NO: 1384 - DNA, 27 bp, Artificial Sequence
  Description of Artificial Sequence Synthetic primer
ttcgcgcatccaggagaagtacatgtt

SEQ ID NO: 1385 - DNA, 15 bp, Artificial Sequence
  Description of Artificial Sequence Synthetic primer
ttcgctcgccgctac

SEQ ID NO: 1386 - DNA, 18 bp, Artificial Sequence
  Description of Artificial Sequence Synthetic primer
ttcgctctcggcctggcc

SEQ ID NO: 1387 - DNA, 22 bp, Artificial Sequence
  Description of Artificial Sequence Synthetic primer
ttcggtataacgcatcgcagca

SEQ ID NO: 1388 - DNA, 26 bp, Artificial Sequence
  Description of Artificial Sequence Synthetic primer
ttcgtgctggattttgtccttgtcct

SEQ ID NO: 1389 - DNA, 21 bp, Artificial Sequence
  Description of Artificial Sequence Synthetic primer
ttcgtgcttagatgctttcag

SEQ ID NO: 1390 - DNA, 24 bp, Artificial Sequence
  Description of Artificial Sequence Synthetic primer
ttctgagctaaatcagcagttgca

SEQ ID NO: 1391 - DNA, 23 bp, Artificial Sequence
  Description of Artificial Sequence Synthetic primer
ttctgcgaatcaatcgcacgctg

SEQ ID NO: 1392 - DNA, 24 bp, Artificial Sequence
  Description of Artificial Sequence Synthetic primer
ttctgcttgaggaatagtgcgtgg

SEQ ID NO: 1393 - DNA, 24 bp, Artificial Sequence
  Description of Artificial Sequence Synthetic primer
ttctgggtgacctggtgttttaga

SEQ ID NO: 1394 - DNA, 31 bp, Artificial Sequence
  Description of Artificial Sequence Synthetic primer
ttcttccaaggatagatttatttcttgttcg

SEQ ID NO: 1395 - DNA, 24 bp, Artificial Sequence
  Description of Artificial Sequence Synthetic primer
ttcttgaacgcgaggtttcgattg

SEQ ID NO: 1396 - DNA, 22 bp, Artificial Sequence
  Description of Artificial Sequence Synthetic primer
ttgacatcgtccctcttcacag

SEQ ID NO: 1397 - DNA, 26 bp, Artificial Sequence
  Description of Artificial Sequence Synthetic primer
ttgacatttgcatgcttcaaagcctg

SEQ ID NO: 1398 - DNA, 23 bp, Artificial Sequence
  Description of Artificial Sequence Synthetic primer
ttgacgtcatccccaccttcctc

SEQ ID NO: 1399 - DNA, 24 bp, Artificial Sequence
  Description of Artificial Sequence Synthetic primer
ttgacgttgcatgttcgagcccat

SEQ ID NO: 1400 - DNA, 30 bp, Artificial Sequence
  Description of Artificial Sequence Synthetic primer
ttgcaatcgacatatccatttcaccatgcc

SEQ ID NO: 1401 - DNA, 27 bp, Artificial Sequence
  Description of Artificial Sequence Synthetic primer
ttgcacgtctgtttcagttgcaaattc

SEQ ID NO: 1402 - DNA, 27 bp, Artificial Sequence
  Description of Artificial Sequence Synthetic primer
ttgcacgtctgtttcagttgcaaattc

SEQ ID NO: 1403 - DNA, 20 bp, Artificial Sequence
  Description of Artificial Sequence Synthetic primer
ttgcatcgggttggtaagtc

SEQ ID NO: 1404 - DNA, 27 bp, Artificial Sequence
  Description of Artificial Sequence Synthetic primer
ttgccactttgacaactcctgttgctg

SEQ ID NO: 1405 - DNA, 25 bp, Artificial Sequence
  Description of Artificial Sequence Synthetic primer
ttgccatagcaaagcctacagcatt

SEQ ID NO: 1406 - DNA, 30 bp, Artificial Sequence
  Description of Artificial Sequence Synthetic primer
ttgccattcatggtatttaagtgtagcaga

SEQ ID NO: 1407 - DNA, 21 bp, Artificial Sequence
  Description of Artificial Sequence Synthetic primer
ttgcgccatacgtaccatcgt

SEQ ID NO: 1408 - DNA, 25 bp, Artificial Sequence
  Description of Artificial Sequence Synthetic primer
ttgcgttgcagattatctttaccaa

SEQ ID NO: 1409 - DNA, 25 bp, Artificial Sequence
  Description of Artificial Sequence Synthetic primer
ttgctgccatagcaaagcctacagc

SEQ ID NO: 1410 - DNA, 30 bp, Artificial Sequence
  Description of Artificial Sequence Synthetic primer
ttgctgctttcgcatggttaatcgcttcaa

SEQ ID NO: 1411 - DNA, 30 bp, Artificial Sequence
  Description of Artificial Sequence Synthetic primer
ttgctgctttcgcatggttaattgcttcaa

SEQ ID NO: 1412 - DNA, 27 bp, Artificial Sequence
  Description of Artificial Sequence Synthetic primer
ttggacctgtaatcagctgaatactgg

SEQ ID NO: 1413 - DNA, 22 bp, Artificial Sequence
  Description of Artificial Sequence Synthetic primer
ttggccatcagaccacgcatac

SEQ ID NO: 1414 - DNA, 22 bp, Artificial Sequence
  Description of Artificial Sequence Synthetic primer
ttggccatcaggccacgcatac

SEQ ID NO: 1415 - DNA, 28 bp, Artificial Sequence
  Description of Artificial Sequence Synthetic primer
ttggcgacggtatacccatagctttata

SEQ ID NO: 1416 - DNA, 17 bp, Artificial Sequence
  Description of Artificial Sequence Synthetic primer
ttggtgcgcttggcgta

SEQ ID NO: 1417 - DNA, 32 bp, Artificial Sequence
  Description of Artificial Sequence Synthetic primer
ttggttcttacttgttttgcataaactttcca

SEQ ID NO: 1418 - DNA, 35 bp, Artificial Sequence
  Description of Artificial Sequence Synthetic primer
ttgtacatttgaaacaatatgcatgacatgtgaat

SEQ ID NO: 1419 - DNA, 23 bp, Artificial Sequence
  Description of Artificial Sequence Synthetic primer
ttgtcagactcatcgcgaacatc

SEQ ID NO: 1420 - DNA, 28 bp, Artificial Sequence
  Description of Artificial Sequence Synthetic primer
ttgtgatatggaggtgtagaaggtgtta

SEQ ID NO: 1421 - DNA, 26 bp, Artificial Sequence
  Description of Artificial Sequence Synthetic primer
ttgtgattgttttgcagctgattgtg

SEQ ID NO: 1422 - DNA, 27 bp, Artificial Sequence
  Description of Artificial Sequence Synthetic primer
ttgtggccgatttcaccacctgctcct

SEQ ID NO: 1423 - DNA, 21 bp, Artificial Sequence
  Description of Artificial Sequence Synthetic primer
ttgttaacggcttcaagaccc

SEQ ID NO: 1424 - DNA, 32 bp, Artificial Sequence
  Description of Artificial Sequence Synthetic primer
ttgtttattgtttccatatgctacacactttc

SEQ ID NO: 1425 - DNA, 23 bp, Artificial Sequence
  Description of Artificial Sequence Synthetic primer
tttaagcgccagaaagcaccaac

SEQ ID NO: 1426 - DNA, 25 bp, Artificial Sequence
  Description of Artificial Sequence Synthetic primer
tttacctcgcctttccacccttacc

SEQ ID NO: 1427 - DNA, 29 bp, Artificial Sequence
  Description of Artificial Sequence Synthetic primer
tttagctactattctagctgccatttcca

SEQ ID NO: 1428 - DNA, 27 bp, Artificial Sequence
  Description of Artificial Sequence Synthetic primer
tttatgaccagcttcggtactactaaa

SEQ ID NO: 1429 - DNA, 30 bp, Artificial Sequence
  Description of Artificial Sequence Synthetic primer
tttatggtctatttcaatggcagttacgaa

SEQ ID NO: 1430 - DNA, 37 bp, Artificial Sequence
  Description of Artificial Sequence Synthetic primer
tttcaatacctttatgcaacttngtatcaacnggaat

SEQ ID NO: 1431 - DNA, 30 bp, Artificial Sequence
  Description of Artificial Sequence Synthetic primer
tttcacagcatgcacgtctgtttcagttgc

SEQ ID NO: 1432 - DNA, 35 bp, Artificial Sequence
  Description of Artificial Sequence Synthetic primer
tttccccgatctaaatttggataagccataggaaa

SEQ ID NO: 1433 - DNA, 27 bp, Artificial Sequence
  Description of Artificial Sequence Synthetic primer
tttccgatgcaacgtaatgagatttca

SEQ ID NO: 1434 - DNA, 22 bp, Artificial Sequence
  Description of Artificial Sequence Synthetic primer
tttcgtgcttagatgctttcag

SEQ ID NO: 1435 - DNA, 30 bp, Artificial Sequence
  Description of Artificial Sequence Synthetic primer
tttcttgaagagtatgagctgctccgtaag

SEQ ID NO: 1436 - DNA, 22 bp, Artificial Sequence
  Description of Artificial Sequence Synthetic primer
tttgcaccttaccgccaaagct

SEQ ID NO: 1437 - DNA, 28 bp, Artificial Sequence
  Description of Artificial Sequence Synthetic primer
tttgctcatgatctgcatgaagcataaa

SEQ ID NO: 1438 - DNA, 24 bp, Artificial Sequence
  Description of Artificial Sequence Synthetic primer
tttgctctccgccaaagtttccac

SEQ ID NO: 1439 - DNA, 28 bp, Artificial Sequence
  Description of Artificial Sequence Synthetic primer
tttggacctgtaatcagctgaatactgg

SEQ ID NO: 1440 - DNA, 29 bp, Artificial Sequence
  Description of Artificial Sequence Synthetic primer
tttgtgaaacagcgaacattttcttggta

SEQ ID NO: 1441 - DNA, 20 bp, Artificial Sequence
  Description of Artificial Sequence Synthetic primer
ttttccagccatgcagcgac

SEQ ID NO: 1442 - DNA, 34 bp, Artificial Sequence
  Description of Artificial Sequence Synthetic primer
ttttccctttatgcaacttagtatcaacnggaat

SEQ ID NO: 1443 - DNA, 29 bp, Artificial Sequence
  Description of Artificial Sequence Synthetic primer
ttttgctcatgatctgcatgaagcataaa

SEQ ID NO: 1444 - DNA, 2118 bp, Artificial Sequence
  Description of Artificial Sequence Synthetic Concatenation of A. baumannii genes
cgcgcggtaaaactaaagaagaagatatagcattagaaaaagatttgctgtctgatgaaaaagagattgctgaacatttaatgctgattgatcttgggcgaaacgatgtagggcgtgtatcgaaaataggtaaagtccaagtcacggatcaaatggtgatcgagcgttattcacatgtcatgcatattgtttcaaatgtacaaggtgaagtgcgtgatgatatcgatgcacttgatgtatttaaagccacctttccagcaggaacgttatcaggtgccccaaaaattcgtgcaatggaaattattgatgaagtagaacctgtgaaaaggggagtttttggcggggctgttggttatttgggatggcatggtgaaatggatatgtcgattgcaatccgtacttgtgttatccgtgataaaaaggtgtatgtacaggctggtgcagggnnnnnnnnnnnnnnnnnnnnnnnnnnnnnnnnnnnnnnnnggaatctggcggtttagtttcagatgaactcattatcggtttagtaaaagaacgtattgctcaacctgactgcgtgaatggttgtattttcgacggcttcccacgcactattcctcaagcagaagctttggaaaaagaagggatcagcattgatcatgtaattgaaattgatgtacctgatgaagaaatcgtaaaacgtctttctggtcgtcgtcagcatcctgcttctggtcgtgtttatcacgttgtatacaatccacctaaagtggaaggtaaagatgatgtcacaggnnnnnnnnnnnnnnnnnnnnnnnnnnnnnnnnnnnnnnnncgttcaaccgtgtaaaattacgtaaccttaaaactggtaaagttttagaaaaaacttttaaatctggtgatactttagaagctgctgacatcgtagaagtagaaatgaactacctatacaacgatggcgaaatgtggcacttcatggacccagaaagcttcgaacaaattgcagctgacaaaactgcaatgggtgatgctgctaaatggttaaaagacgactcaaatgaaacatgtacaatcatgttattcaacggcgttcctttaaacgtaaatgcacctaacttcgttgtattgaaagttgttgaaactgatccgggcgtacgtggtgatacttctggtggtnnnnnnnnnnnnnnnnnnnnnnnnnnnnnnnnnnnnnnnntcgtgcccgyaatttgcataaagctgccggccttgtagcacagcaaggcaaatttcctgaaactctagaagaatggattgcactacccggcattggtcgctcgaccgcaggtgcactcatgtctttaggtttacgtcagtatggcgtgattatggatggcaacgtgaaacgcgttttagcccgtttctttgccattgaagatgacttaagcaaaccacagcacgaacgtgaaatgtggaaactggctgaagagctttgtcccacccaacgcaatcatgactacactcaagcgannnnnnnnnnnnnnnnnnnnnnnnnnnnnnnnnnnnnnnnttaaaaacactagcggtaagcttaaacaagattgccaatgatattcgttggttagcaagtggtccacgttgcggcttcggcgaaatccgtattcctgaaaatgaacctggttcaagtatcatgccaggtaaagtgaacccgactcaaagtgaagccatgaccatggttgttgctcaagtacttggcaacgataccactattaatgtcgctggtgcttctggtaacttcgagctcaatgtatttatgccagtgattgcttataacttactgcaatctattcagttgcttggtgatgcatgtaatagttttaatgatcactgtgcagtagggatcgagccaaatcgtgagaaaattgatcatttcttgcataattctcttatgttagttacggcannnnnnnnnnnnnnnnnnnnnnnnnnnnnnnnnnnnnnnncccggttatgtaccaaatactttgtctgaagatggtgacccattagacgtacttgttgtaactccacatcctgttgctgccggttctgtaattcgttgccgcccagtgggcaaattaaacatggaagacgacggtggtatcgatgccnnnn

SEQ ID NO: 1445 - DNA, 102 bp, Artificial Sequence
  Description of Artificial Sequence Synthetic Calibration Polynucleotide
tttaagtcccgcaacgagcgcaacccttgatcttagttgtttagttgggcactctaaggtgactgccggtgacaaaccggaggaaggtggggatgacgtcaa

SEQ ID NO: 1446 - DNA, 94 bp, Artificial Sequence
  Description of Artificial Sequence Synthetic Calibration Polynucleotide
tagaacaccgatggcgaaggcgactttctggtctgtaactgacactgagaaagcgtggggagcaaacaggattagataccctggtagtccacga

SEQ ID NO: 1447 - DNA, 108 bp, Artificial Sequence
  Description of Artificial Sequence Synthetic Calibration Polynucleotide
tggattagagaccctggtagtccacgccgtaaacgatgagtgctaagtgttagaggcctttagtgctgaagttaacgcattaagcactccgcctggggagtacggcca

SEQ ID NO: 1448 - DNA, 108 bp, Artificial Sequence
  Description of Artificial Sequence Synthetic Calibration Polynucleotide
tttcgatgcaacgcgaagaaccttaccaggtcttgacatcctctgacaaccctagcttctccttcgggagcagagtgacaggtggtgcatggctgtcgtcagctcgta

SEQ ID NO: 1449 - DNA, 95 bp, Artificial Sequence
  Description of Artificial Sequence Synthetic Calibration Polynucleotide
tctgacacctgcccggtgctggaaggttaaggagaggggttagcgtaactctgaactgaagccccagtaaacggcggccgtaactataacggtca

SEQ ID NO: 1450 - DNA, 117 bp, Artificial Sequence
  Description of Artificial Sequence Synthetic Calibration Polynucleotide
tctgttcttagtacgagaggaccgggatggacgcaccggtaccagttgttctgccaagggcatagctgggtagctatgtgcggaagggataagtgctgaaagcatctaagcacgaaa

SEQ ID NO: 1451 - DNA, 100 bp, Artificial Sequence
  Description of Artificial Sequence Synthetic Calibration Polynucleotide
tgattattgttatcctgttatgccatttgagatttttgagtggtattggagttattgttccaggattaattgcaaatacaattcaaagacaagggttaca

SEQ ID NO: 1452 - DNA, 112 bp, Artificial Sequence
  Description of Artificial Sequence Synthetic Calibration Polynucleotide
tcgaagtacaatacaagacaaaagaaggtaaaattactgttttaggggaaaaattcaagaaatatagaagtgatggctaaaaatgtagaaggggtcttgaagccgttaacaa

SEQ ID NO: 1453 - DNA, 100 bp, Artificial Sequence
  Description of Artificial Sequence Synthetic Calibration Polynucleotide
ttgctcgtggtgcacaagtaacggatattacaatcattgttgttgcagctgatgacggcgtaataaacagttgaagcaattaaccatgcgaaagcagcaa

SEQ ID NO: 1454 - DNA, 114 bp, Artificial Sequence
  Description of Artificial Sequence Synthetic Calibration Polynucleotide
tagcttttgcatattatatcgagccacagcatcgtgatgttttacagctttatgcaccggaagcttttaatggataaatttaacgaacaagaaataaatctatccttggaagaa

SEQ ID NO: 1455 - DNA, 116 bp, Artificial Sequence
  Description of Artificial Sequence Synthetic Calibration Polynucleotide
tgacctacagtaagaggttctgtaatgaaccctaatgaccatccacacggtggtggtgaaggtagatctcctatcggaaagtccacgtactccatggggtaaaccagcacttggaa

SEQ ID NO: 1456 - DNA, 70 bp, Artificial Sequence
  Description of Artificial Sequence Synthetic Calibration Polynucleotide
tccacacggtggtggtgaaggtagatctcctatcggaaagtccacgtactccatggggtaaaccagcaca

SEQ ID NO: 1457 - DNA, 82 bp, Artificial Sequence
  Description of Artificial Sequence Synthetic Calibration Polynucleotide
ttatcgctcaggcgaactccaacctggatgatgaaggccgctttttagaaggtgacttgtcgtagcaaaggcgaatccagca

SEQ ID NO: 1458 - DNA, 87 bp, Artificial Sequence
  Description of Artificial Sequence Synthetic Calibration Polynucleotide
tgggcagcgtttcggcgaaatggaagtggctcgaagcgtatggcgcttcgtacgtgctgcaggaaatgttgaccgtcaagtcggaca

SEQ ID NO: 1459 - DNA, 97 bp, Artificial Sequence
  Description of Artificial Sequence Synthetic Calibration Polynucleotide
tcaggagtcgttcaactcgatctacatgatggccgaccgcccggggttcggcggtgcagattcgtcagctggccggcatgcgtggcctgatggcgta

SEQ ID NO: 1460 - DNA, 117 bp, Artificial Sequence
  Description of Artificial Sequence Synthetic Calibration Polynucleotide
tctggcaggtatgcgtggtctgatggccaatccatctggtcgtatcatcgaacttccaatcaagtttccgtgaaggtttaacagtacttgagtacttcatctcaacccacggtgcga

SEQ ID NO: 1461 - DNA, 98 bp, Artificial Sequence
  Description of Artificial Sequence Synthetic Calibration Polynucleotide
tcaagcaaacgcacaatcagaagctaagaaagcgcaagcttctggaaagcacaaatgctagttatggtacagaatttgcaactgaaacagacgtgcaa

SEQ ID NO: 1462 - DNA, 99 bp, Artificial Sequence
  Description of Artificial Sequence Synthetic Calibration Polynucleotide
tccacacgccgttcttcaacaactaccgtgttctacttccgtacgacggacgtgacgggctcgatcgagctgccgaaggacaaggaaatggtgatgcca

SEQ ID NO: 1463 - DNA, 111 bp, Artificial Sequence
  Description of Artificial Sequence Synthetic Calibration Polynucleotide
tcgtggcggcgtggttatcgaacccatgctgaccgatcaatggtacgtgcacaccgccccccaaagtcgcgattgaagccgtagagaacggcgacatccagttcgtaccga

SEQ ID NO: 1464 - DNA, 2100 bp, Artificial Sequence
  Description of Artificial Sequence Synthetic Combination Calibration Polynucleotide
gaagtagagatatggaggaacaccagtggcgaaggcgactttctggtctgtaactgacactgagaaagcgtggggagcaaacaggattagataccctggtagtccacgccgtaaacgatgagtgctaagtgttagaggcctttagtgctgaagttaacgcattaagcactccgcctggggagtacggccgcaaggctgaaactcaaaggaattgacggggcacaagcggtggagcatgtggtttaattcgaagcaacgcgaagaaccttaccaggtcttgacatcctctgacaaccctagcttctccttcgggagcagagtgacaggtggtgcatggttgtcgtcagctcgtgtcgtgagatgttgggttaagtcccgcaacgagcgcaacccttgatcttagttgtttagttgggcactctaaggtgactgccggtgacaaaccggaggaaggtggggatgacgtcaaatcatcatgccccagtaccgtgagggaaaggtgaaaagcaccccggaaggggagtgaaagagatcctgaaaccgtgtgccatagtcagagcccgttaacgggtgatggcgtgccttttgtagaatgaaccggcgagttataagatccgtagtcaaaagggaaacagcccagaccgccagctaaggtcccaaagtgtgtattgaaaaggatgtggagttgcttagacaactaggatgttggcttagaagcagccaccatttaaagagtatagggggtgacacctgcccggtgctggaaggttaaggagaggggttagcgtaactctgaactgaagccccagtaaacggcggccgtaactataacggtcctaaggtagcgaaagaaatttgagaggagctgtccttagtacgagaggaccgggatggacgcaccggtaccagttgttctgccaagggcatagctgggtagctatgtgcggaagggataagtgctgaaagcatctaagcatgaagcccccctcaagatgagagcagtaaaacaagcaaacgcacaatcagaagctaagaaagcgcaagcttctggaaagcacaaatgctagttatggtacagaatttgcaactgaaacagacgtgcatgctgtgaaatttgcgaaagcttttgcatattatatcgagccacagcatcgtgatgttttacagctttatgcaccggaagcttttaatggataaatttaacgaacaagaaataaatctatccttggaagaacttaaagatcaacggatgctggcaagatatgaaaaataagataaaacagcactatcaacactggagcgattctttatctgaagaaggaagagcgatgaaaacaacgaagtacaatacaagacaaaagaaggtaaaattactgttttaggggaaaaattcaagaaatatagaagtgatggctaaaaatgtagaaggggtcttgaagccgttaacagctgttatggcgaccgtggcggcgtggttatcgaacccatgctgaccgatcaatggtacgtgcacaccgccccccaaagtcgcgattgaagccgtagagaacggcgagatccagttcgtccctaaacagtacggcaacttcgttatcgctcaggcgaactccaacctggatgatgaaggccgctttttagaaggtgacttgtcgtagcaaaggcgaatcaagcctgtttagccacaactatgcgtgctcgtggtgcacaagtaacggatattacaatcattgttgttgcagctgatgacggcgtaataaacagttgaagcgattaaccatgcgaaagcagcaggagtaccaactttactcagcttgctggtatgcgtggtctgatggccaatccatctggtcgtatcatcgaacttccaatcaagtttccgtgaaggtttaacagtacttgagtacttcatctctacgcatggtgcgcgtaaaggtcatgggagtaagacctacagtaagaggttctgtaatgaaccctaatgaccatccacacggtggtggtgaaggtagatctcctatcggaaagtccacgtactccatggggtaaaccagcacttggatacaaaacaagcgcagttcggcggccagcgcttcggtgaaa

SEQ ID NO: 1465 - DNA, 28 bp, Artificial Sequence
  Description of Artificial Sequence Synthetic primer
tcgtcatcagctaactcaaatacatgga

SEQ ID NO: 1466 - DNA, 28 bp, Artificial Sequence
  Description of Artificial Sequence Synthetic primer
tcactttgatatgtggatccgtcattca

SEQ ID NO: 1467 - DNA, 23 bp, Artificial Sequence
  Description of Artificial Sequence Synthetic primer
taagccctttgttgcttgcgaca

SEQ ID NO: 1468 - DNA, 28 bp, Artificial Sequence
  Description of Artificial Sequence Synthetic primer
tcagacccactactataccagtctagca

SEQ ID NO: 1469 - DNA, 24 bp, Artificial Sequence
  Description of Artificial Sequence Synthetic primer
tgccaacatactagcgaaggaact

SEQ ID NO: 1470 - DNA, 31 bp, Artificial Sequence
  Description of Artificial Sequence Synthetic primer
tcccatgaaccttaacttttaaaggtagttc

SEQ ID NO: 1471 - DNA, 17 bp, Artificial Sequence
  Description of Artificial Sequence Synthetic primer
acgcgaagaaccttacc

SEQ ID NO: 1472 - DNA, 14 bp, Artificial Sequence
  Description of Artificial Sequence Synthetic primer
cgaagaaccttacc

SEQ ID NO: 1473 - DNA, 12 bp, Artificial Sequence
  Description of Artificial Sequence Synthetic primer
acacgagctgac

SEQ ID NO: 1474 - DNA, 15 bp, Artificial Sequence
  Description of Artificial Sequence Synthetic primer
tgtgctttgaatgct

SEQ ID NO: 1475 - DNA, 19 bp, Artificial Sequence
  Description of Artificial Sequence Synthetic primer
tgcttctggtgctagcatt

SEQ ID NO: 1476 - DNA, 20 bp, Artificial Sequence
  Description of Artificial Sequence Synthetic primer
tcatttgtgctttgaatgct

SEQ ID NO: 1477 - DNA, 24 bp, Artificial Sequence
  Description of Artificial Sequence Synthetic primer
tcaagcaaacgcacaatcagaagc

SEQ ID NO: 1478 - DNA, 27 bp, Artificial Sequence
  Description of Artificial Sequence Synthetic primer
tcttgtttatgctggtaaagcagatgg

SEQ ID NO: 1479 - DNA, 31 bp, Artificial Sequence; identical to SEQ ID NO: 476
  Description of Artificial Sequence Synthetic primer

SEQ ID NO: 1480 - DNA, 29 bp, Artificial Sequence
  Description of Artificial Sequence Synthetic primer
tgtaacccttgtctttgaattgtatttgc

(19) United States
(12) Patent Application Publication
(10) Pub. No.: US 2011/0256541 A1
(43) Pub. Date: Oct. 20, 2011

(54) COMPOSITIONS FOR USE IN IDENTIFICATION OF BACTERIA

(76) Inventor: David J. Ecker, Encinitas, CA (US)

(21) Appl. No.: 12/532,809

(22) PCT Filed: Mar. 20, 2008
(86) PCT No.: PCT/US08/57717

§ 371 Date: Jun. 27, 2011

Related U.S. Application Data
(60) Provisional application No. 60/896,813, filed on Mar. 23, 2007.
(60) Provisional application No. 60/896,822, filed on Mar. 23, 2007.

(51) Int. Cl. C12Q 1/68

(52) U.S. Cl. 435/6.12

(57) ABSTRACT

The present invention provides compositions, kits and methods for rapid identification and quantification of bacteria by molecular mass and base composition analysis.

### CROSS-REFERENCE TO RELATED APPLICATIONS

This application is a U.S. National Phase application under 35 U.S.C. §371 claiming priority to International Application Number PCT/US2008/057717 filed on Mar. 20, 2008 under the Patent Cooperation Treaty, which claims the benefit of priority to U.S. Provisional Application Ser. Nos. 60/896,813, filed Mar. 23, 2007 and 60/896,822, filed Mar. 23, 2007, the disclosures of which are incorporated by reference in their entirety for any purpose.

### STATEMENT OF GOVERNMENT SUPPORT

This invention was made with United States Government support under CDC contract RO1 CI000099-01. The United States Government has certain rights in the invention.

### SEQUENCE LISTING

Computer-readable forms of the sequence listing, on CD-ROM, containing the file named DIBIS0096WOSEQ.txt, which is 257,746 bytes (measured in MS-DOS), and were created on Mar. 20, 2008, are herein incorporated by reference.

### FIELD OF THE INVENTION

The present invention provides compositions, kits and methods for rapid identification and quantification of bacteria by molecular mass and base composition analysis.

### BACKGROUND OF THE INVENTION

A problem in determining the cause of a natural infectious outbreak or a bioterrorist attack is the sheer variety of organisms that can cause human disease. There are over 1400 organisms infectious to humans; many of these have the potential to emerge suddenly in a natural epidemic or to be used in a malicious attack by bioterrorists (Taylor et al. Philos. Trans. R. Soc. London B. Biol. Sci., 2001, 356, 983-989). This number does not include numerous strain variants, bioengineered versions, or pathogens that infect plants or animals.

Much of the new technology being developed for detection of biological weapons incorporates a polymerase chain reaction (PCR) step based upon the use of highly specific primers and probes designed to selectively detect certain pathogenic organisms. Although this approach is appropriate for the most obvious bioterrorist organisms, like smallpox and anthrax, experience has shown that it is very difficult to predict which of hundreds of possible pathogenic organisms might be employed in a terrorist attack. Likewise, naturally emerging human disease that has caused devastating consequence in public health has come from unexpected families of bacteria, viruses, fungi, or protozoa. Plants and animals also have their natural burden of infectious disease agents and there are equally important biosafety and security concerns for agriculture.

A major conundrum in public health protection, biodefense, and agricultural safety and security is that these disciplines need to be able to rapidly identify and characterize infectious agents, while there is no existing technology with the breadth of function to meet this need. Currently used methods for identification of bacteria rely upon culturing the bacterium to effect isolation from other organisms and to obtain sufficient quantities of nucleic acid followed by sequencing of the nucleic acid, both processes which are time and labor intensive.

Mass spectrometry provides detailed information about the molecules being analyzed, including high mass accuracy. It is also a process that can be easily automated. DNA chips with specific probes can only determine the presence or absence of specifically anticipated organisms. Because there are hundreds of thousands of species of benign bacteria, some very similar in sequence to threat organisms, even arrays with 10,000 probes lack the breadth needed to identify a particular organism.

Provided herein are oligonucleotide primers and compositions and kits containing the oligonucleotide primers, which define bacterial bioagent identifying amplicons and, upon amplification, produce corresponding amplification products whose molecular masses provide the means to identify bacteria, for example, at and below the species taxonomic level.

### SUMMARY OF THE INVENTION

Provided herein are, inter alia, oligonucleotide primers, oligonucleotide primer pairs, compositions and kits comprising the same, and methods for their use in rapid identification, characterization and quantification of bacteria (also referred to herein as bacterial bioagents) by molecular mass and base composition analysis. In one embodiment, the bacteria are members of the Staphylococcus genus. In a preferred embodiment, they are members of the Staphylococcus aureus species. The forward and reverse primer members of the oligonucleotide primer pairs are configured to amplify one or more nucleic acids from bioagents, thereby generating amplicons (amplification products) for the nucleic acids. In one embodiment, the primers generate bioagent identifying nucleic acid amplicons. The amplicons are preferably generated from gene sequences within the nucleic acid.

Each of the oligonucleotide primer pairs comprises a forward and a reverse primer. In a preferred embodiment, each of the forward and reverse primers comprises between 13 and 35 linked nucleotides in length. Thus, in this embodiment, the primer may comprise 13, 14, 15, 16, 17, 18, 19, 20, 21, 22, 23, 24, 25, 26, 27, 28, 29, 30, 31, 32, 33, 34, or 35 linked nucleotides in length.

In a preferred embodiment, the forward primer of the oligonucleotide primer pair comprises between 70% and 100% sequence identity with SEQ ID NO.: 1465. In one aspect, the forward primer comprises at least 70% sequence identity with SEQ ID NO.: 1465. In another aspect, the forward primer comprises at least 80% sequence identity with SEQ ID NO.: 1465. In another aspect, the forward primer comprises at least 90% sequence identity with SEQ ID NO.: 1465. In another aspect, the forward primer comprises at least 95% sequence identity with SEQ ID NO.: 1465. In another aspect, the forward primer comprises at least 100% sequence identity with SEQ ID NO.: 1465. In another aspect, the forward primer is SEQ ID NO.: 1465 with 0-10 nucleotide deletions, additions, and/or substitutions. In another aspect, the forward primer is SEQ ID NO.: 1465.

In embodiment, the reverse primer of the oligonucleotide primer pair comprises between 70% and 100% sequence identity with SEQ ID NO.: 1466. In one aspect, the reverse primer comprises at least 70% sequence identity with SEQ ID NO.: 1466. In another aspect, the reverse primer comprises at least 80% sequence identity with SEQ ID NO.: 1466. In another aspect, the reverse primer comprises at least 90% sequence identity with SEQ ID NO.: 1466. In another aspect, the reverse primer comprises at least 95% sequence identity with SEQ ID NO.: 1466. In another aspect, the reverse primer comprises at least 100% sequence identity with SEQ ID NO.: 1466. In another aspect, the reverse primer is SEQ ID NO.: 1466 with 0-10 nucleotide deletions, additions, and/or substitutions. In another aspect, the reverse primer is SEQ ID NO.: 1466.

One embodiment is an oligonucleotide primer between 13 and 35 linked nucleotides in length having at least 70% sequence identity with SEQ ID NO: 1465.

Another embodiment is an oligonucleotide primer between 13 and 35 linked nucleotides in length having at least 70% sequence identity with SEQ ID NO: 1466.

Another embodiment is an oligonucleotide primer pair wherein the forward primer is between 13 and 35 linked nucleotides in length and comprises at least 70% sequence identity with SEQ ID NO: 1465 and an the reverse primer is between 13 and 35 linked nucleotides in length and comprises at least 70% sequence identity with SEQ ID NO: 1466.

One embodiment is an oligonucleotide primer between 13 and 35 linked nucleotides in length having at least 70% sequence identity with SEQ ID NO: 288.

Another embodiment is an oligonucleotide primer between 13 and 35 linked nucleotides in length having at least 70% sequence identity with SEQ ID NO: 1269.

Another embodiment is an oligonucleotide primer pair wherein the forward primer is between 13 and 35 linked nucleotides in length and comprises at least 70% sequence identity with SEQ ID NO: 288 and an the reverse primer is between 13 and 35 linked nucleotides in length and comprises at least 70% sequence identity with SEQ ID NO: 1269.

One embodiment is an oligonucleotide primer between 13 and 35 linked nucleotides in length having at least 70% sequence identity with SEQ ID NO: 698.

Another embodiment is an oligonucleotide primer between 13 and 35 linked nucleotides in length having at least 70% sequence identity with SEQ ID NO: 1420.

Another embodiment is an oligonucleotide primer pair wherein the forward primer is between 13 and 35 linked nucleotides in length and comprises at least 70% sequence identity with SEQ ID NO: 698 and the reverse primer is between 13 and 35 linked nucleotides in length and comprises at least 70% sequence identity with SEQ ID NO: 1420.

One embodiment is an oligonucleotide primer between 13 and 35 linked nucleotides in length having at least 70% sequence identity with SEQ ID NO: 217.

Another embodiment is an oligonucleotide primer between 13 and 35 linked nucleotides in length having at least 70% sequence identity with SEQ ID NO: 1167

Another embodiment is an oligonucleotide primer pair wherein the forward primer is between 13 and 35 linked nucleotides in length and comprises at least 70% sequence identity with SEQ ID NO: 217 and wherein the reverse primer is between 13 and 35 linked nucleotides in length and comprises at least 70% sequence identity with SEQ ID NO: 1167.

One embodiment is an oligonucleotide primer between 13 and 35 linked nucleotides in length having at least 70% sequence identity with SEQ ID NO: 399.

Another embodiment is an oligonucleotide primer between 13 and 35 linked nucleotides in length having at least 70% sequence identity with SEQ ID NO: 1041.

Another embodiment is an oligonucleotide primer pair wherein the forward primer is between 13 and 35 linked nucleotides in length and comprises at least 70% sequence identity with SEQ ID NO: 399 and wherein the reverse primer is between 13 and 35 linked nucleotides in length and comprises at least 70% sequence identity with SEQ ID NO: 1041.

One embodiment is an oligonucleotide primer between 13 and 35 linked nucleotides in length having at least 70% sequence identity with SEQ ID NO: 430.

Another embodiment is an oligonucleotide primer between 13 and 35 linked nucleotides in length having at least 70% sequence identity with SEQ ID NO: 1321.

Another embodiment is an oligonucleotide primer pair wherein the forward primer is between 13 and 35 linked nucleotides in length and comprises at least 70% sequence identity with SEQ ID NO: 430 and the reverse primer is between 13 and 35 linked nucleotides in length and comprises at least 70% sequence identity with SEQ ID NO: 1321.

One embodiment is an oligonucleotide primer between 13 and 35 linked nucleotides in length having at least 70% sequence identity with SEQ ID NO: 174.

Another embodiment is an oligonucleotide primer between 13 and 35 linked nucleotides in length having at least 70% sequence identity with SEQ ID NO: 853.

Another embodiment is an oligonucleotide primer pair wherein the forward primer is between 13 and 35 linked nucleotides in length and comprises at least 70% sequence identity with SEQ ID NO: 174 and the reverse primer is between 13 and 35 linked nucleotides in length and comprises at least 70% sequence identity with SEQ ID NO: 853.

One embodiment is an oligonucleotide primer between 13 and 35 linked nucleotides in length having at least 70% sequence identity with SEQ ID NO: 172.

Another embodiment is an oligonucleotide primer between 13 and 35 linked nucleotides in length having at least 70% sequence identity with SEQ ID NO: 1360.

Another embodiment is an oligonucleotide primer pair wherein the forward primer is between 13 and 35 linked nucleotides in length and comprises at least 70% sequence identity with SEQ ID NO: 172 and the reverse primer is between 13 and 35 linked nucleotides in length and comprises at least 70% sequence identity with SEQ ID NO: 1360.

One embodiment is an oligonucleotide primer between 13 and 35 linked nucleotides in length having at least 70% sequence identity with SEQ ID NO: 205.

Another embodiment is an oligonucleotide primer between 13 and 35 linked nucleotides in length having at least 70% sequence identity with SEQ ID NO: 876.

Another embodiment is an oligonucleotide primer pair wherein the forward primer is between 13 and 35 linked nucleotides in length and comprises at least 70% sequence identity with SEQ ID NO: 205 and the reverse primer is between 13 and 35 linked nucleotides in length and comprises at least 70% sequence identity with SEQ ID NO: 876.

Another embodiment is an oligonucleotide primer pair 13 to 35 linked nucleotides in length having at least 70% sequence identity with SEQ ID NO.: 456.

Another embodiment is an oligonucleotide primer pair 13 to 35 linked nucleotides in length having at least 70% sequence identity with SEQ ID NO.: 1261.

Another embodiment is an oligonucleotide primer pair wherein the forward primer is between 13 and 35 linked nucleotides in length and comprises at least 70% sequence identity with SEQ ID NO: 456 and the reverse primer is between 13 and 35 linked nucleotides in length and comprises at least 70% sequence identity with SEQ ID NO: 1261.

Another embodiment is an oligonucleotide primer pair 13 to 35 linked nucleotides in length having at least 70% sequence identity with SEQ ID NO.: 437.

Another embodiment is an oligonucleotide primer pair 13 to 35 linked nucleotides in length having at least 70% sequence identity with SEQ ID NO.: 1137.

Another embodiment is an oligonucleotide primer pair 13 to 35 linked nucleotides in length having at least 70% sequence identity with SEQ ID NO.: 1231.

Another embodiment is an oligonucleotide primer pair wherein the forward primer is between 13 and 35 linked nucleotides in length and comprises at least 70% sequence identity with SEQ ID NO: 456 and the reverse primer is between 13 and 35 linked nucleotides in length and comprises at least 70% sequence identity with SEQ ID NO: 1231 or with SEQ ID NO.: 1137.

Another embodiment is an oligonucleotide primer pair 13 to 35 linked nucleotides in length having at least 70% sequence identity with SEQ ID NO.: 530.

Another embodiment is an oligonucleotide primer pair 13 to 35 linked nucleotides in length having at least 70% sequence identity with SEQ ID NO.: 891.

Another embodiment is an oligonucleotide primer pair wherein the forward primer is between 13 and 35 linked nucleotides in length and comprises at least 70% sequence identity with SEQ ID NO: 530 and the reverse primer is between 13 and 35 linked nucleotides in length and comprises at least 70% sequence identity with SEQ ID NO: 891.

Another embodiment is an oligonucleotide primer pair 13 to 35 linked nucleotides in length having at least 70% sequence identity with SEQ ID NO.: 474.

Another embodiment is an oligonucleotide primer pair 13 to 35 linked nucleotides in length having at least 70% sequence identity with SEQ ID NO.: 869.

Another embodiment is an oligonucleotide primer pair wherein the forward primer is between 13 and 35 linked nucleotides in length and comprises at least 70% sequence identity with SEQ ID NO: 474 and the reverse primer is between 13 and 35 linked nucleotides in length and comprises at least 70% sequence identity with SEQ ID NO: 869.

Another embodiment is an oligonucleotide primer pair 13 to 35 linked nucleotides in length having at least 70% sequence identity with SEQ ID NO.: 268.

Another embodiment is an oligonucleotide primer pair 13 to 35 linked nucleotides in length having at least 70% sequence identity with SEQ ID NO.: 1284.

Another embodiment is an oligonucleotide primer pair wherein the forward primer is between 13 and 35 linked nucleotides in length and comprises at least 70% sequence identity with SEQ ID NO: 268 and the reverse primer is between 13 and 35 linked nucleotides in length and comprises at least 70% sequence identity with SEQ ID NO: 1284.

Another embodiment is an oligonucleotide primer pair 13 to 35 linked nucleotides in length having at least 70% sequence identity with SEQ ID NO.: 418.

Another embodiment is an oligonucleotide primer pair 13 to 35 linked nucleotides in length having at least 70% sequence identity with SEQ ID NO.: 1301.

Another embodiment is an oligonucleotide primer pair wherein the forward primer is between 13 and 35 linked nucleotides in length and comprises at least 70% sequence identity with SEQ ID NO: 418 and the reverse primer is between 13 and 35 linked nucleotides in length and comprises at least 70% sequence identity with SEQ ID NO: 1301.

Another embodiment is an oligonucleotide primer pair 13 to 35 linked nucleotides in length having at least 70% sequence identity with SEQ ID NO.: 318.

Another embodiment is an oligonucleotide primer pair 13 to 35 linked nucleotides in length having at least 70% sequence identity with SEQ ID NO.: 1300.

Another embodiment is an oligonucleotide primer pair wherein the forward primer is between 13 and 35 linked nucleotides in length and comprises at least 70% sequence identity with SEQ ID NO: 318 and the reverse primer is between 13 and 35 linked nucleotides in length and comprises at least 70% sequence identity with SEQ ID NO: 1300.

Another embodiment is an oligonucleotide primer pair 13 to 35 linked nucleotides in length having at least 70% sequence identity with SEQ ID NO.: 440.

Another embodiment is an oligonucleotide primer pair 13 to 35 linked nucleotides in length having at least 70% sequence identity with SEQ ID NO.: 1076.

Another embodiment is an oligonucleotide primer pair wherein the forward primer is between 13 and 35 linked nucleotides in length and comprises at least 70% sequence identity with SEQ ID NO: 440 and the reverse primer is between 13 and 35 linked nucleotides in length and comprises at least 70% sequence identity with SEQ ID NO: 1076.

Another embodiment is an oligonucleotide primer pair 13 to 35 linked nucleotides in length having at least 70% sequence identity with SEQ ID NO.: 219.

Another embodiment is an oligonucleotide primer pair 13 to 35 linked nucleotides in length having at least 70% sequence identity with SEQ ID NO.: 1013.

Another embodiment is an oligonucleotide primer pair wherein the forward primer is between 13 and 35 linked nucleotides in length and comprises at least 70% sequence identity with SEQ ID NO: 219 and the reverse primer is between 13 and 35 linked nucleotides in length and comprises at least 70% sequence identity with SEQ ID NO: 1013.

Also provided herein are kits comprising one or more of the oligonucleotide primer pairs. In one embodiment, the kit comprises an oligonucleotide primer pair comprising a forward primer that comprises at least 70% sequence identity with SEQ ID NO.: 1465 and a reverse primer that comprises at least 70% sequence identity with SEQ ID NO.: 1466, the forward primer comprises at least 70% sequence identity with SEQ ID NO.: 1467 and the reverse primer comprises at least 70% sequence identity with SEQ ID NO.: 1468, or the forward primer comprises at least 70% sequence identity with SEQ ID NO.: 1469 and the reverse primer comprises at least 70% sequence identity with SEQ ID NO.: 1470. In a preferred embodiment, the primer pair comprises at least 70% sequence identity with SEQ ID NO.: 1465:SEQ ID NO.: 1466, SEQ ID NO.: 1467:SEQ ID NO.:1468, or SEQ ID NO.: 1469:SEQ ID NO.:1470. In another embodiment, the kit comprises at least one additional oligonucleotide primer pair that is configured to generate an amplicon between 45 and 200 linked nucleotides in length, and comprises a forward and a reverse primer, each comprising between 13 and 35 linked nucleotides in length and each configured to hybridize to conserved sequence regions within a Staphylococcus aureus gene, said gene selected from the group consisting of: ermA, ermC, pvluk, nuc, tufB, mecA, mec-R1, tsst1, and mupR, arcC, aroE, gmk, pta, tpi and yqi. In a preferred embodiment, each of the at least one additional oligonucleotide primer pair comprises at least 70% sequence identity with a primer pair selected from: SEQ ID NO.: 288:SEQ ID NO.:1269, SEQ ID NO.: 698:SEQ ID NO.:1420, SEQ ID NO.: 217:SEQ ID NO.:1167, SEQ ID NO.: 399:SEQ ID NO.:1041, SEQ ID NO.: 456:SEQ ID NO.:1261, SEQ ID NO.: 430:SEQ ID NO.:1321, SEQ ID NO.: 174:SEQ ID NO.:853, SEQ ID NO.: 437:SEQ ID NO.:1232, SEQ ID NO.: 530:SEQ ID NO.:891, SEQ ID NO.: 474:SEQ ID NO.:869, SEQ ID NO.: 268:SEQ ID NO.:1284, SEQ ID NO.: 418:SEQ ID NO.:1301, SEQ ID NO.: 318:SEQ ID NO.:1300, SEQ ID NO.: 440:SEQ ID NO.:1076, and SEQ ID NO.: 219:SEQ ID NO.:1013. In another aspect, the kit comprises eight primer pairs, said eight oligonucleotide primer pairs having at least 70% sequence identity to: SEQ ID NO.: 288:SEQ ID NO.:1269, SEQ ID NO.: 698:SEQ ID NO.:1420, SEQ ID NO.: 217:SEQ ID NO.:1167, SEQ ID NO.: 399:SEQ ID NO.:1041, SEQ ID NO.: 456:SEQ ID NO.:1261, SEQ ID NO.: 430:SEQ ID NO.:1321, SEQ ID NO.: 174:SEQ ID NO.:853, and SEQ ID NO.: 1465:SEQ ID NO.:1466, SEQ ID NO.: 1467:SEQ ID NO.:1468, or SEQ ID NO.: 1469:SEQ ID NO.:1470. In another aspect, the kit comprises eight oligonucleotide primer pairs consisting of SEQ ID NO.: 288:SEQ ID NO.:1269, SEQ ID NO.: 698:SEQ ID NO.:1420, SEQ ID NO.: 217:SEQ ID NO.:1167, SEQ ID NO.: 399:SEQ ID NO.:1041, SEQ ID NO.: 456:SEQ ID NO.:1261, SEQ ID NO.: 430:SEQ ID NO.:1321, SEQ ID NO.: 174:SEQ ID NO.:853, and SEQ ID NO.: 1465:SEQ ID NO.:1466, SEQ ID NO.: 1467:SEQ ID NO.:1468, or SEQ ID NO.: 1469:SEQ ID NO.:1470. In one aspect, the kit further comprises eight additional primer pairs, comprising at least 70% sequence identity with SEQ ID NO.: 437:SEQ ID NO.:1232, SEQ ID NO.: 530:SEQ ID NO.:891, SEQ ID NO.: 474:SEQ ID NO.:869, SEQ ID NO.: 268:SEQ ID NO.:1284, SEQ ID NO.: 418:SEQ ID NO.:1301, SEQ ID NO.: 318:SEQ ID NO.:1300, SEQ ID NO.: 440:SEQ ID NO.:1076, and SEQ ID NO.: 219:SEQ ID NO.:1013. In another aspect, the eight additional primer pairs consists of: SEQ ID NO.: 437:SEQ ID NO.:1232, SEQ ID NO.: 530:SEQ ID NO.:891, SEQ ID NO.: 474:SEQ ID NO.:869, SEQ ID NO.: 268:SEQ ID NO.:1284, SEQ ID NO.: 418:SEQ ID NO.:1301, SEQ ID NO.: 318:SEQ ID NO.:1300, SEQ ID NO.: 440:SEQ ID NO.:1076, and SEQ ID NO.: 219:SEQ ID NO.:1013.

In a preferred embodiment, the kit comprises A kit for identifying a Staphylococcus aureus bioagent comprising: a first oligonucleotide primer pair comprising a forward primer with at least 70% sequence identity with: SEQ ID NO.: 288 and a reverse primer with at least 70% sequence identity with SEQ ID NO.:1269; a second oligonucleotide primer pair comprising a forward primer with at least 70% sequence identity with SEQ ID NO.: 698 and a reverse primer with at least 70% sequence identity with SEQ ID NO.:1420; a third oligonucleotide primer pair comprising a forward primer with at least 70% sequence identity with: SEQ ID NO.: 217 and a reverse primer with at least 70% sequence identity with: SEQ ID NO.:1167; a fourth oligonucleotide primer pair comprising a forward primer with at least 70% sequence identity with: SEQ ID NO.: 399 and a reverse primer with at least 70% sequence identity with: SEQ ID NO.:1041; a fifth oligonucleotide primer pair comprising a forward primer with at least 70% sequence identity with: SEQ ID NO.: 456 and a reverse primer with at least 70% sequence identity with: SEQ ID NO.:1261; a sixth oligonucleotide primer pair comprising a forward primer with at least 70% sequence identity with: SEQ ID NO.: 430 and a reverse primer with at least 70% sequence identity with: SEQ ID NO.:1321; a seventh oligonucleotide primer pair comprising a forward primer with at least 70% sequence identity with: SEQ ID NO.: 174 and a reverse primer with at least 70% sequence identity with: SEQ ID NO.:853; and an eighth oligonucleotide primer pair comprising a forward primer with at least 70% sequence identity with: SEQ ID NO.: 172 and a reverse primer with at least 70% sequence identity with: SEQ ID NO.:1360.

In another preferred embodiment, the kit comprises the eight oligonucleotide primer pairs: SEQ ID NO.: 288:SEQ ID NO.:1269, SEQ ID NO.: 698:SEQ ID NO.:1420, SEQ ID NO.: 217:SEQ ID NO.:1167, SEQ ID NO.: 399:SEQ ID NO.:1041, SEQ ID NO.: 456:SEQ ID NO.:1261, SEQ ID NO.: 430:SEQ ID NO.:1321, SEQ ID NO.: 174:SEQ ID NO.:853, and SEQ ID NO.: 172:SEQ ID NO.:1360.

In another preferred embodiment, the kit for identifying a Staphylococcus aureus bioagent comprises:a first oligonucleotide primer pair comprising a forward primer with at least 70% sequence identity with: SEQ ID NO.: 288 and a reverse primer with at least 70% sequence identity with SEQ ID NO.:1269, a second oligonucleotide primer pair comprising a forward primer with at least 70% sequence identity with: SEQ ID NO.: 698 and a reverse primer with at least 70% sequence identity with: SEQ ID NO.:1420, a third oligonucleotide primer pair comprising a forward primer with at least 70% sequence identity with: SEQ ID NO.: 217 and a reverse primer with at least 70% sequence identity with: SEQ ID NO.:1167, a fourth oligonucleotide primer pair comprising a forward primer with at least 70% sequence identity with: SEQ ID NO.: 399 and a reverse primer with at least 70% sequence identity with: SEQ ID NO.:1041, a fifth oligonucleotide primer pair comprising a forward primer with at least 70% sequence identity with: SEQ ID NO.: 456, and a reverse primer with at least 70% sequence identity with: SEQ ID NO.:1261, a sixth oligonucleotide primer pair comprising a forward primer with at least 70% sequence identity with: SEQ ID NO.: 430 and a reverse primer with at least 70% sequence identity with: SEQ ID NO.:1321, a seventh oligonucleotide primer pair comprising a forward primer with at least 70% sequence identity with: SEQ ID NO.: 174 and a reverse primer with at least 70% sequence identity with: SEQ ID NO.:853; and an eighth oligonucleotide primer pair comprising a forward primer with at least 70% sequence identity with: SEQ ID NO.: 205 and a reverse primer with at least 70% sequence identity with: SEQ ID NO.:876.

In a preferred embodiment, the kit comprises eight oligonucleotide primer pairs consisting of: SEQ ID NO.: 288:SEQ ID NO.:1269, SEQ ID NO.: 698:SEQ ID NO.:1420, SEQ ID NO.: 217:SEQ ID NO.:1167, SEQ ID NO.: 399:SEQ ID NO.:1041, SEQ ID NO.: 456:SEQ ID NO.:1261, SEQ ID NO.: 430:SEQ ID NO.:1321, SEQ ID NO.: 174:SEQ ID NO.:853, and SEQ ID NO.: 205:SEQ ID NO.:876.

In another preferred embodiment, the kit for identifying a Staphylococcus aureus bioagent comprises: a first oligonucleotide primer pair comprising a forward primer with at least 70% sequence identity with: SEQ ID NO.: 288 and a reverse primer with at least 70% sequence identity with: SEQ ID NO.:1269, a second oligonucleotide primer pair comprising a forward primer with at least 70% sequence identity with: SEQ ID NO.: 698 and a reverse primer with at least 70% sequence identity with: SEQ ID NO.:1420, a third oligonucleotide primer pair comprising a forward primer with at least 70% sequence identity with: SEQ ID NO.: 217 and a reverse primer with at least 70% sequence identity with: SEQ ID NO.:1167, a fourth oligonucleotide primer pair comprising a forward primer with at least 70% sequence identity with: SEQ ID NO.: 399 and a reverse primer with at least 70% sequence identity with: SEQ ID NO.:1041, a fifth oligonucleotide primer pair comprising a forward primer with at least 70% sequence identity with: SEQ ID NO.: 456 and a reverse primer with at least 70% sequence identity with: SEQ ID NO.:1261, a sixth oligonucleotide primer pair comprising a forward primer with at least 70% sequence identity with: SEQ ID NO.: 430 and a reverse primer with at least 70% sequence identity with: SEQ ID NO.:1321, a seventh oligonucleotide primer pair comprising a forward primer with at least 70% sequence identity with: SEQ ID NO.: 174 and a reverse primer with at least 70% sequence identity with: SEQ ID NO.:853; and an eighth oligonucleotide primer pair comprising a forward primer with at least 70% sequence identity with: SEQ ID NO.: 1465 and a reverse primer with at least 70% sequence identity with: SEQ ID NO.:1466.

In a preferred embodiment, the kit comprises eight oligonucleotide primer pairs consisting of: SEQ ID NO.: 288:SEQ ID NO.:1269, SEQ ID NO.: 698:SEQ ID NO.:1420, SEQ ID NO.: 217:SEQ ID NO.:1167, SEQ ID NO.: 399:SEQ ID NO.:1041, SEQ ID NO.: 456:SEQ ID NO.:1261, SEQ ID NO.: 430:SEQ ID NO.:1321, SEQ ID NO.: 174:SEQ ID NO.:853, and SEQ ID NO.: 1465:SEQ ID NO.:1466.

In another preferred embodiment, the for identifying a Staphylococcus aureus bioagent comprises: a first oligonucleotide primer pair comprising a forward primer with at least 70% sequence identity with: SEQ ID NO.: 437 and a primer with at least 70% sequence identity with:SEQ ID NO.:1137, a second oligonucleotide primer pair comprising a forward primer with at least 70% sequence identity with: SEQ ID NO.: 530 and a reverse primer with at least 70% sequence identity with:SEQ ID NO.:891, a third oligonucleotide primer pair comprising a forward primer with at least 70% sequence identity with: SEQ ID NO.: 474 and a reverse primer with at least 70% sequence identity with: SEQ ID NO.:869, a fourth oligonucleotide primer pair comprising a forward primer with at least 70% sequence identity with: SEQ ID NO.: 268 and a reverse primer with at least 70% sequence identity with: SEQ ID NO.:1284, a fifth oligonucleotide primer pair comprising a forward primer with at least 70% sequence identity with: SEQ ID NO.: 418 and a reverse primer with at least 70% sequence identity with: SEQ ID NO.:1301, a sixth oligonucleotide primer pair comprising a forward primer with at least 70% sequence identity with: SEQ ID NO.: 318 and a reverse primer with at least 70% sequence identity with: SEQ ID NO.:1300, a seventh oligonucleotide primer pair comprising a forward primer with at least 70% sequence identity with: SEQ ID NO.: 440 and a reverse primer with at least 70% sequence identity with: SEQ ID NO.:1076, and an eigth oligonucleotide primer pair comprising a forward primer with at least 70% sequence identity with: SEQ ID NO.: 219 and a reverse primer with at least 70% sequence identity with: SEQ ID NO.:1013.

In a preferred embodiment, the kit comprises eight oligonucleotide primer pairs consisting of: SEQ ID NO.: 437:SEQ ID NO.:1137, SEQ ID NO.: 530:SEQ ID NO.:891, SEQ ID NO.: 474:SEQ ID NO.:869, SEQ ID NO.: 268:SEQ ID NO.:1284, SEQ ID NO.: 418:SEQ ID NO.:1301, SEQ ID NO.: 318:SEQ ID NO.:1300, SEQ ID NO.: 440:SEQ ID NO.:1076, and SEQ ID NO.: 219:SEQ ID NO.:1013.

In another preferred embodiment, the kit for identifying a Staphylococcus aureus bioagent comprises: a first oligonucleotide primer pair comprising a forward primer with at least 70% sequence identity with: SEQ ID NO.: 437 and a reverse primer with at least 70% sequence identity with: SEQ ID NO.:1232, a second oligonucleotide primer pair comprising a forward primer with at least 70% sequence identity with: SEQ ID NO.: 530 and a reverse primer with at least 70% sequence identity with: SEQ ID NO.:891, a third oligonucleotide primer pair comprising a forward primer with at least 70% sequence identity with: SEQ ID NO.: 474 and a reverse primer with at least 70% sequence identity with: SEQ ID NO.:869, a fourth oligonucleotide primer pair comprising a forward primer with at least 70% sequence identity with: SEQ ID NO.: 268 and a reverse primer with at least 70% sequence identity with: SEQ ID NO.:1284, a fifth oligonucleotide primer pair comprising a forward primer with at least 70% sequence identity with: SEQ ID NO.: 418 and a reverse primer with at least 70% sequence identity with: SEQ ID NO.:1301, a sixth oligonucleotide primer pair comprising a forward primer with at least 70% sequence identity with: SEQ ID NO.: 318 and a reverse primer with at least 70% sequence identity with: SEQ ID NO.:1300, a seventh oligonucleotide primer pair comprising a forward primer with at least 70% sequence identity with: SEQ ID NO.: 440 and a reverse primer with at least 70% sequence identity with: SEQ ID NO.:1076; and an eighth oligonucleotide primer pair comprising a forward primer with at least 70% sequence identity with: SEQ ID NO.: 219 and a reverse primer with at least 70% sequence identity with: SEQ ID NO.:1013.

In a preferred embodiment, the kit comprises eight oligonucleotide primer pairs consisting of: SEQ ID NO.: 437:SEQ ID NO.:1232, SEQ ID NO.: 530:SEQ ID NO.:891, SEQ ID NO.: 474:SEQ ID NO.:869, SEQ ID NO.: 268:SEQ ID NO.:1284, SEQ ID NO.: 418:SEQ ID NO.:1301, SEQ ID NO.: 318:SEQ ID NO.:1300, SEQ ID NO.: 440:SEQ ID NO.:1076, and SEQ ID NO.: 219:SEQ ID NO.:1013.

In another preferred embodiment, the kit for identifying a Staphylococcus aureus bioagent comprises: a first oligonucleotide primer pair comprising a forward primer with at least 70% sequence identity with: SEQ ID NO.: 437 and a reverse primer with at least 70% sequence identity with: SEQ ID NO.:1232, a second oligonucleotide primer pair comprising a forward primer with at least 70% sequence identity with: SEQ ID NO.: 530 and a reverse primer with at least 70% sequence identity with: SEQ ID NO.:891, a third oligonucleotide primer pair comprising a forward primer with at least 70% sequence identity with: SEQ ID NO.: 474 and a reverse primer with at least 70% sequence identity with: SEQ ID NO.:869, a fourth oligonucleotide primer pair comprising a forward primer with at least 70% sequence identity with: SEQ ID NO.: 268 and a reverse primer with at least 70% sequence identity with: SEQ ID NO.:1284, a fifth oligonucleotide primer pair comprising a forward primer with at least 70% sequence identity with: SEQ ID NO.: 418 and a reverse primer with at least 70% sequence identity with: SEQ ID NO.:1301, a sixth oligonucleotide primer pair comprising a forward primer with at least 70% sequence identity with: SEQ ID NO.: 318 and a reverse primer with at least 70% sequence identity with: SEQ ID NO.:1300, a seventh oligonucleotide primer pair comprising a forward primer with at least 70% sequence identity with: SEQ ID NO.: 440 and a reverse primer with at least 70% sequence identity with: SEQ ID NO.:1076, an eighth oligonucleotide primer pair comprising a forward primer with at least 70% sequence identity with: SEQ ID NO.: 219 and a reverse primer with at least 70% sequence identity with: SEQ ID NO.:1013, a ninth oligonucleotide primer pair comprising a forward primer with at least 70% sequence identity with: SEQ ID NO.: 288 and a reverse primer with at least 70% sequence identity with: SEQ ID NO.:1269, a tenth oligonucleotide primer pair comprising a forward primer with at least 70% sequence identity with: SEQ ID NO.: 698 and a reverse primer with at least 70% sequence identity with: SEQ ID NO.:1420, an eleventh oligonucleotide primer pair comprising a forward primer with at least 70% sequence identity with: SEQ ID NO.: 217 and a reverse primer with at least 70% sequence identity with: SEQ ID NO.:1167, a twelfth oligonucleotide primer pair comprising a forward primer with at least 70% sequence identity with: SEQ ID NO.: 399 and a reverse primer with at least 70% sequence identity with: SEQ ID NO.:1041, a thirteenth oligonucleotide primer pair comprising a forward primer with at least 70% sequence identity with: SEQ ID NO.: 456 and a reverse primer with at least 70% sequence identity with: SEQ ID NO.:1261, a fourteenth oligonucleotide primer pair comprising a forward primer with at least 70% sequence identity with: SEQ ID NO.: 430 and a reverse primer with at least 70% sequence identity with: SEQ ID NO.:1321, a fifteenth oligonucleotide primer pair comprising a forward primer with at least 70% sequence identity with: SEQ ID NO.: 174 and a reverse primer with at least 70% sequence identity with: SEQ ID NO.:853; and a sixteenth oligonucleotide primer pair comprising a forward primer with at least 70% sequence identity with: SEQ ID NO.: 205 and a reverse primer with at least 70% sequence identity with: SEQ ID NO.:876.

Preferably, each of the oligonucleotide primer pairs is configured to generate an amplicon comprising between 45 and 200 linked nucleotides in length, and wherein, for each of the oligonucleotide primer pairs, the forward primer comprises between 13 and 35 linked nucleotides in length and is configured to hybridize within a first conserved sequence region of a Staphylococcus aureus gene sequence, and the reverse primer comprises between 13 and 35 linked nucleotides in length and is configured to hybridize within a second conserved sequence region of said Staphylococcus aureus gene sequence.

In some embodiments, at least one of the forward primer and the reverse primer comprises at least one modified nucleobase. In one embodiment, at least one of the at least one modified nucleobase is a mass modified nucleobase. In one aspect, the mass modified nucleobase is 5-Iodo-C. In another aspect, it comprises a mass modified tag. In another embodiment, at least one of the at least one modified nucleobase is a universal nucleobase, for example, inosine. In another embodiment, primer pair comprises at least one non-templated T residue on the 5′-end. In another embodiment, at least one of the forward primer and the reverse primer comprises at least one non-template tag. In one embodiment, at least one of the forward primer and the reverse primer comprises a non-templated T residue on the 5′-end. In another embodiment, at least one of the forward primer and the reverse primer lacks a non-templated T residue on the 5′-end.

Some embodiments are kits that comprise one or more of the primer pairs. In some embodiments, each member of the one or more primer pairs of the kit is of a length of between 13 and 35 linked nucleotides and has 70% to 100% sequence identity with the corresponding member from any of the primer pairs listed in Table 2.

In some embodiments, the kits comprise at least one calibration polynucleotide for use in quantitiation of bacteria in a given sample, and also for use as a positive control for amplification.

In some embodiments, the kits further comprise at least one anion exchange functional group linked to a magnetic bead.

Also provided herein are methods for identification of bacteria using one or more of the primer pairs provided herein. In one embodiment, the method is for identification of a bioagent in a sample. In one aspect, the bioagent is a bacterial bioagent, preferably a Staphylococcus aureus bioagent. Nucleic acid from the sample is amplified using the oligonucleotide primer pairs described above to obtain at least one amplification product. In a preferred aspect, the amplification product is between 45 and 200 linked nucleotides in length. The molecular mass of the amplification product is determined by mass spectrometry. In a preferred embodiment, the base composition of the amplification product is calculated from the determined molecular mass. The molecular mass and/or base composition is compared to or queried against a database comprising a plurality of base compositions or molecular masses. Preferably, each base composition/molecular mass within the plurality of base compositions and/or molecular masses in the database is indexed to the primer pair and to a bioagent. A match between the calculated base composition or the determined molecular mass with a base composition or molecular mass comprised in the database identifies the bioagent in the sample. In preferred embodiments, the mass spectrometry used to determine the molecular mass is electrospray ionization (ESI) time of flight (TOF) mass spectrometry or ESI Fourier transform ion cyclotron resonance (FTICR) mass spectrometry, for example. Other mass spectrometry techniques can also be used to measure the molecular mass of bacterial bioagent identifying amplicons.

In some embodiments, the identification in the method comprises detecting the presence or absence of a bacterial bioagent in a sample. In another embodiment, it comprises determining the presence or absence of virulence of the bioagent in the sample. In another embodiment, the identifying comprises identifying one or more sub-species characteristics of the bioagent in the sample. In another embodiment, the identifying comprises determining sensitivity or resistance of the bioagent to a drug, preferably an antibiotic.

In some embodiments, the methods are for determination of the quantity of an unknown bacterial bioagent in a sample. The sample is contacted with the primer pair and a known quantity of a calibration polynucleotide comprising a calibration sequence. Nucleic acid from the unknown bioagent in the sample is concurrently amplified with the composition described above and nucleic acid from the calibration polynucleotide in the sample is concurrently amplified with the composition described above to obtain a first amplification product comprising a bacterial bioagent identifying amplicon and a second amplification product comprising a calibration amplicon. The molecular masses and abundances for the bacterial bioagent identifying amplicon and the calibration amplicon are determined. The bacterial bioagent identifying amplicon is distinguished from the calibration amplicon based on molecular mass and comparison of bacterial bioagent identifying amplicon abundance and calibration amplicon abundance indicates the quantity of bacterium in the sample. In some embodiments, the base composition of the bacterial bioagent identifying amplicon is determined.

In some embodiments, the methods comprise detecting or quantifying bacteria by combining a nucleic acid amplification process with molecular mass determination. In some embodiments, such methods identify or otherwise analyze the bacterium by comparing mass information from an amplification product with a calibration or control product. Such methods can be carried out in a highly multiplexed and/or parallel manner allowing for the analysis of as many as 300 samples per 24 hours on a single mass measurement platform. The accuracy of the mass determination methods in some embodiments provided herein permits allows for the ability to discriminate between different bacteria such as, for example, various genotypes and drug resistant strains of Staphylococcus aureus.

### BRIEF DESCRIPTION OF THE DRAWINGS

The foregoing summary and the following detailed description are better understood when read in conjunction with the accompanying drawings which are included by way of example and not by way of limitation.

FIG. 1: process diagram illustrating a representative primer pair selection process.

FIG. 2: process diagram illustrating an embodiment of the calibration method.

FIG. 3: common pathogenic bacteria and primer pair coverage. The primer pair number in the upper right hand corner of each polygon indicates that the primer pair can produce a bioagent identifying amplicon for all species within that polygon.

FIG. 4: a representative 3D diagram of base composition (axes A, G and C) of bioagent identifying amplicons obtained with primer pair number 14 (a precursor of primer pair number 348 which targets 16S rRNA). The diagram indicates that the experimentally determined base compositions of the clinical samples (labeled NHRC samples) closely match the base compositions expected for Streptococcus pyogenes and are distinct from the expected base compositions of other organisms.

FIG. 5: a representative mass spectrum of amplification products indicating the presence of bioagent identifying amplicons of Streptococcus pyogenes, Neisseria meningitidis, and Haemophilus influenzae obtained from amplification of nucleic acid from a clinical sample with primer pair number 349 which targets 23S rRNA. Experimentally determined molecular masses and base compositions for the sense strand of each amplification product are shown.

FIG. 6: a representative mass spectrum of amplification products representing a bioagent identifying amplicon of Streptococcus pyogenes, and a calibration amplicon obtained from amplification of nucleic acid from a clinical sample with primer pair number 356 which targets rp1B. The experimentally determined molecular mass and base composition for the sense strand of the Streptococcus pyogenes amplification product is shown.

FIG. 7: a representative mass spectrum of an amplified nucleic acid mixture which contained the Ames strain of Bacillus anthracis, a known quantity of combination calibration polynucleotide (SEQ ID NO: 1464), and primer pair number 350 which targets the capC gene on the virulence plasmid pX02 of Bacillus anthracis. Calibration amplicons produced in the amplification reaction are visible in the mass spectrum as indicated and abundance data (peak height) are used to calculate the quantity of the Ames strain of Bacillus anthracis.

### DETAILED DESCRIPTION OF THE EMBODIMENTS

As used herein, the term “abundance” refers to an amount. The amount may be described in terms of concentration which are common in molecular biology such as “copy number,” “pfu or plate-forming unit” which are well known to those with ordinary skill. Concentration may be relative to a known standard or may be absolute. In some embodiments, the primer pairs and methods provided herein determine the abundance of one or more bioagents in a sample.

As used herein, the term “amplifiable nucleic acid” is used in reference to nucleic acids that may be amplified by any amplification method. It is contemplated that “amplifiable nucleic acid” also comprises “sample template.”

As used herein, the term “amplification reagents” refers to those reagents (deoxyribonucleotide triphosphates, buffer, etc.), needed for amplification, excluding primers, nucleic acid template, and the amplification enzyme. Typically, amplification reagents along with other reaction components are placed and contained in a reaction vessel (test tube, microwell, etc.).

As used herein, the term “analogous” when used in context of comparison of bioagent identifying amplicons indicates that the bioagent identifying amplicons being compared are produced with the same pair of primers. For example, bioagent identifying amplicon “A” and bioagent identifying amplicon “B”, produced with the same pair of primers are analogous with respect to each other. Bioagent identifying amplicon “C”, produced with a different pair of primers is not analogous to either bioagent identifying amplicon “A” or bioagent identifying amplicon “B”.

As used herein, the term “anion exchange functional group” refers to a positively charged functional group capable of binding an anion through an electrostatic interaction. The most well known anion exchange functional groups are the amines, including primary, secondary, tertiary and quaternary amines.

The term “bacteria” or “bacterium” refers to any member of the groups of eubacteria and archaebacteria.

As used herein, a “base composition probability cloud” is a representation of the diversity in base composition resulting from a variation in sequence that occurs among different isolates of a given species. The “base composition probability cloud” represents the base composition constraints for each species and is typically visualized using a pseudo four-dimensional plot.

Herein, a “bioagent” is any organism, cell, or virus, living or dead, or a nucleic acid derived from such an organism, cell or virus. Examples of bioagents include, but are not limited, to cells, (including but not limited to human clinical samples, bacterial cells and other pathogens), viruses, fungi, protists, parasites, and pathogenicity markers (including but not limited to: pathogenicity islands, antibiotic resistance genes, virulence factors, toxin genes and other bioregulating compounds). Samples may be alive or dead or in a vegetative state (for example, vegetative bacteria or spores) and may be encapsulated or bioengineered. Herein, a “pathogen” is a bioagent which causes a disease or disorder.

As is used herein, the term “unknown bioagent” can mean either: (i) a bioagent whose existence is not known (for example, the SARS coronavirus was unknown prior to April 2003), which is also called a “true unknown bioagent,” and/or (ii) a bioagent whose existence is known (such as the well known bacterial species Staphylococcus aureus for example) but which is not known to be in a sample to be analyzed and/or (iii) a bioagent that is known or suspected of being present in a sample but whose sub-species characteristics are not known (such as a bacterial resistance genotype like the QRDR region of Staphyoicoccus aureus species). For example, if the method for identification of coronaviruses disclosed in commonly owned U.S. Pre-Grant Publication No. US2005-0266397 (incorporated herein by reference in its entirety) was to be employed prior to April 2003 to identify the SARS coronavirus in a clinical sample, both meanings of “unknown” bioagent are applicable since the SARS coronavirus was unknown to science prior to April, 2003 and since it was not known what bioagent (in this case a coronavirus) was present in the sample. On the other hand, if the method of U.S. Pre-Grant Publication No. US2005-0266397 was to be employed subsequent to April 2003 to identify the SARS coronavirus in a clinical sample, only the second meaning (ii) of “unknown” bioagent would apply because the SARS coronavirus became known to science subsequent to April 2003 but because it was not known what bioagent was present in the sample.

As used herein, a “bioagent division” is defined as group of bioagents above the species level and includes but is not limited to, orders, families, genus, classes, clades, genera or other such groupings of bioagents above the species level.

Herein, a “pathogen” is a bioagent which causes a disease or disorder.

The term “virus” refers to obligate, ultramicroscopic, parasites that are incapable of autonomous replication (i.e., replication requires the use of the host cell's machinery). Viruses can survive outside of a host cell but cannot replicate.

As used herein, the term “biological product” refers to any product originating from an organism. Biological products are often products of processes of biotechnology. Examples of biological products include, but are not limited to: cultured cell lines, cellular components, antibodies, proteins and other cell-derived biomolecules, growth media, growth harvest fluids, natural products and bio-pharmaceutical products.

The terms “biowarfare agent” and “bioweapon” are synonymous and refer to a bacterium, virus, fungus or protozoan that could be deployed as a weapon to cause bodily harm to individuals. Military or terrorist groups may be implicated in deployment of biowarfare agents.

The term “calibration amplicon” refers to a nucleic acid segment representing an amplification product obtained by amplification of a calibration sequence with a pair of primers configured to produce a bioagent identifying amplicon.

The term “calibration sequence” refers to a polynucleotide sequence to which a given pair of primers hybridizes for the purpose of producing an internal (i.e: included in the reaction) calibration standard amplification product for use in determining the quantity of a bioagent in a sample. The calibration sequence may be expressly added to an amplification reaction, or may already be present in the sample prior to analysis.

The term “codon” refers to a set of three adjoined nucleotides (triplet) that codes for an amino acid or a termination signal.

Herein, the term “codon base composition analysis,” refers to determination of the base composition of an individual codon by obtaining a bioagent identifying amplicon that includes the codon. The bioagent identifying amplicon will at least include regions of the target nucleic acid sequence to which the primers hybridize for generation of the bioagent identifying amplicon as well as the codon being analyzed, located between the two primer hybridization regions.

As used herein, “primer pairs,” or “oligonucleotide primer pairs” are synonymous terms referring to pairs of oligonucleotides (herein called “primers” or “oligonucleotide primers”) that are configured to bind to conserved sequence regions of a bioagent nucleic acid (that is conserved among two or more bioagents) and to generate bioagent identifying amplicons. The bound primers flank an intervening variable region of the bioagent between the conserved sequence sequences. Upon amplification, the primer pairs yield amplicons that provide base composition variability between two or more bioagents. The variability of the base compositions allows for the identification of one or more individual bioagents from two or more bioagents based on the base composition distinctions. The primer pairs are also configured to generate amplicons that are amenable to molecular mass analysis. Each primer pair comprises two primer pair members. The primer pair members are a “forward primer” (“forward primer pair member,” or “reverse member”), which comprises at least a percentage of sequence identity with the top strand of the reference sequence used in configuring the primer pair, and a “reverse primer” (“reverse primer pair member” or “reverse member”), which comprises at least a percentage of reverse complementarity with the top strand of the reference sequence used in configuring the primer pair. Primer pair configuration is well known in the art and is described in detail herein.

Primer pair nomenclature, as used herein, includes the identification of a reference sequence. For example, the forward primer for primer pair number 3106 is named TSST1_NC002758.2-2137509-2138213_{—}519_{—}546_F. This forward primer name indicates that the forward primer (“_F”) hybridizes to residues 234-261 (“234_{—}261”) of a reference sequence, which in this case is represented by a sequence extraction of coordinates 2137509-2138213 from GenBank gi number 57634611 (corresponding to the GenBank number NC002758.2, as is indicated by the prefix “TSST1_NC002758.2” and cross-reference in Table 3). In the case of this primer, the reference sequence is the gene within a Staphylococcus aureus genome encoding for tsst1. Primer pair name codes for the primers provided herein are defined in Table 3, which lists gene abbreviations and GenBank gi numbers that correspond with each primer name code. Sequences of the primers are also provided. One of skill in the art will understand how to determine exact hybridization coordinates of primers with respect to GenBank sequences, given the information provided herein. The primer pairs are selected and configured; however, to hybridize with two or more bioagents. So, the reference sequence in the primer name is used merely to provide a reference, and not to indicate that the primers are selected and configured to hybridize with and generate a bioagent identifying amplicon only from the reference sequence. Rather, the primers hybridize with and generate amplicons from a number of sequences. Further, the sequences of the primer members of the primer pairs are not necessarily fully complementary to the conserved region of the reference bioagent. Rather, the sequences are configured to be “best fit” amongst a plurality of bioagents at these conserved binding sequences. Therefore, the primer members of the primer pairs have substantial complementarity with the conserved regions of the bioagents, including the reference bioagent.

The primers provided herein are configured to hybridize within conserved sequence regions of bioagent nucleic acids, which are conserved among two or more bioagents, that preferably flank an intervening variable region, which varies among two or more bioagents, and, upon amplification, yield amplification products which ideally provide enough variability to distinguish individual bioagents, and which are amenable to molecular mass analysis. In a preferred embodiment, the conserved sequence regions are highly conserved sequence regions. By the term “highly conserved,” it is meant that the sequence regions exhibit between about 80-100%, or between about 90-100%, or between about 95-100% identity among all, or at least 70%, at least 80%, at least 90%, at least 95%, or at least 99% of species or strains. The molecular mass of a given amplification product provides a means of identifying the bioagent from which it was obtained, due to the variability of the variable region, which preferably results in amplicons that vary in base composition among bioagents, for example, among different species or strains. Thus configuring of the primers involves selection of a variable region with appropriate variability to resolve the identity of a given bioagent. Bioagent identifying amplicons are ideally specific to the identity of the bioagent.

As used herein, the term “variable region” is used to describe a region that is flanked by the two conserved sequence regions to which the primers of a primer pair hybridize. In other words, the variable region is a region that is flanked by the primers of any one primer pair described herein. The region possesses distinct base compositions among at least two bioagents, such that at least one bioagent can be identified at the family, genus, species or sub-species level using the primer pairs and the methods provided herein. The degree of variability between the at least two bioagents need only be sufficient to allow for identification using mass spectrometry analysis, as described herein. Such a difference can be as slight as a single nucleotide difference occurring between two bioagents.

Methods of oligonucleotide primer pair design are well known. One of skill in the art will understand that primer pairs configured to prime amplification of a double stranded sequence are configured and named using one strand of the double stranded sequence as a reference. The forward primer is the primer of the pair that comprises full or partial sequence identity to the one strand (usually the coding, or sense strand) of the sequence being used as a reference. The reverse primer is the primer of the pair that comprises reverse complementarity to the one strand of the sequence being used as a reference.

In one embodiment, the “plus” or “top” strand (the primary sequence as submitted to GenBank) of the nucleic acid to which the primers hybridize is used as a reference when designing primer pairs. In this case, the forward primer will comprise identity and the reverse primer will comprise reverse complementarity, to the sequence listed in GenBank for the reference sequence. In some embodiments, the primer pair is configured using the “minus” or “bottom” strand (reverse complement of the primary sequence as submitted to and listed in GenBank). In this case, the forward primer comprises sequence identity to the minus strand, and thus comprises reverse complementarity to the top strand, the sequence listed in GenBank. Similarly, in this case, the reverse primer comprises reverse complementarity to the minus strang, and thus comprises identity to the top strand. The ordinarily skilled artisan will know how to design the primers provided herein armed with this disclosure.

In a preferred embodiment, the primer pairs may be configured to generate an amplicon from “within a region of” a particular SEQ ID NO., which may comprise a specific region of the Genbank gi No. to which the primers were configured. Configuring a primer pair to generate an amplicon from “within a region” of a particular nucleic acid means that each primer of the pair hybridizes to a portion of the reference sequence that is within that region. However, one of ordinary skill in the art of primer design will understand that shifting the coordinates of the portion of a reference sequence to which one or both primers hybridizes slightly, in one direction or the other relative to the region given, such that the portion is not entirely within the region, will often result in an equally effective primer pair. Such primer pairs are also encompassed by this description.

The term “Glade primer pair” refers to a primer pair configured to produce bioagent identifying amplicons for species belonging to a clade group. A clade primer pair may also be considered as a “speciating” primer pair which is useful for distinguishing among closely related species.

In some embodiments, the primer pairs comprise “broad range survey primers,” primers configured to identify an unknown bioagent as a member of a particular division (e.g., an order, family, class, clade, or genus). However, in some cases the broad range survey primers are also able to identify unknown bioagents at the species or sub-species level. In other embodiments, the primer pairs comprise “division-wide primers,” configured to identify a bioagent at the species level. In some embodiments, the primer pairs comprise “drill-down” primers, configured to identify a bioagent at the sub-species level. As used herein, the “sub-species” level of identification includes, but is not limited to, strains, subtypes, variants, and isolates. Drill-down primers are not always required for identification at the sub-species level because broad range survey intelligent primers may, in some cases provide sufficient identification resolution to accomplishing this identification objective.

Herein, the term “speciating primer pair” refers to a primer pair configured to produce a bioagent identifying amplicon with the diagnostic capability of identifying species members of a group of genera or a particular genus of bioagents. Primer pair number 2249 (SEQ ID NOs: 430:1321), for example, is a speciating primer pair used to distinguish Staphylococcus aureus from other species of the genus Staphylococcus.

As used herein, a “sub-species characteristic” is a genetic characteristic that provides the means to distinguish two members of the same bioagent species. For example, one viral strain could be distinguished from another viral strain of the same species by possessing a genetic change (e.g., for example, a nucleotide deletion, addition or substitution) in one of the viral genes, such as the RNA-dependent RNA polymerase. Sub-species characteristics such as virulence genes and drug-are responsible for the phenotypic differences among the different strains of bacteria.

Properties of the primers may include any number of properties related to structure including, but not limited to: nucleobase length which may be contiguous (linked together) or non-contiguous (for example, two or more contiguous segments which are joined by a linker or loop moiety), modified or universal nucleobases (used for specific purposes such as for example, increasing hybridization affinity, preventing non-templated adenylation and modifying molecular mass) percent complementarity to a given target sequences.

As used herein, the terms “complementary” or “complementarity” are used in reference to polynucleotides (i.e., a sequence of nucleotides such as an oligonucleotide or a target nucleic acid) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

The term “complement of a nucleic acid sequence” as used herein refers to an oligonucleotide which, when aligned with the nucleic acid sequence such that the 5′ end of one sequence is paired with the 3′ end of the other, is in “antiparallel association.” Complementarity relates to base pairing ability. A nucleobase that is complementary to another nucleobase can base pair with that other nucleobase. Certain bases not commonly found in natural nucleic acids may be included in the nucleic acids provided herein, and include, for example, inosine and 7-deazaguanine Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. Where a first oligonucleotide is complementary to a region of a target nucleic acid and a second oligonucleotide has complementary to the same region (or a portion of this region) a “region of overlap” exists along the target nucleic acid. The degree of overlap will vary depending upon the extent of the complementarity.

As is used herein, the term “substantial complementarity” means that a primer member of a primer pair comprises between about 70%-100%, or between about 80-100%, or between about 90-100%, or between about 95-100% identity, or between about 99-100% sequence identity with the conserved binding sequence of any given bioagent. These ranges of identity are inclusive of all whole or partial numbers embraced within the recited range numbers. For example, and not limitation, 75.667%, 82%, 91.2435% and 97% sequence identity are all numbers that fall within the above recited range of 70% to 100%, therefore forming a part of this description.

As used herein, the terms “amplicon” and “bioagent identifying amplicon” refer to a nucleic acid generated using the primer pairs described herein. The amplicon is preferably double stranded DNA; however, it may be RNA and/or DNA:RNA. The amplicon comprises the sequences of the conserved regions/primer pairs and the intervening variable region. Since the primer pairs provided herein are configured such that two or more different bioagents, when amplified with a given primer pair, will yield amplicons with unique base composition signatures, the base composition signatures can be used to identify bioagents based on association with amplicons. As discussed herein, primer pairs are configured to generate amplicons from two or more bioagents. As such, the base composition of any given amplicon will include the primer pair, the complement of the primer pair, the conserved regions and the variable region from the bioagent that was amplified to generate the amplicon. One skilled in the art understands that the incorporation of the configured primer pair sequences into any amplicon will replace the native bioagent sequences at the primer binding site, and complement thereof. After amplification of the target region using the primers the resultant amplicons having the primer sequences generate the molecular mass data. Amplicons having any native bioagent sequences at the primer binding sites, or complement thereof, are undetectable because of their low abundance. Such is accounted for when identifying one or more bioagents using any particular primer pair. The amplicon further comprises a length that is compatible with mass spectrometry analysis. Bioagent identifying amplicons generate base composition signatures that are preferably unique to the identity of a bioagent.

As used herein, the term “molecular mass” refers to the mass of a compound as determined using mass spectrometry. Herein, the compound is preferably a nucleic acid, more preferably a double stranded nucleic acid, still more preferably a double stranded DNA nucleic acid and is most preferably an amplicon. When the nucleic acid is double stranded the molecular mass is determined for both strands. Here, the strands are separated either before introduction into the mass spectrometer, or the strands are separated by the mass spectrometer (for example, electro-spray ionization will separate the hybridized strands). The molecular mass of each strand is measured by the mass spectrometer. The term “mass spectrometry” refers to measurement of the mass of atoms or molecules. The molecules are first converted to ions, which are separated using electric or magnetic fields according to the ratio of their mass to electric charge. The measured masses are used to identity the molecules.

As used herein, the term “base composition” refers to the number of each residue comprising an amplicon, without consideration for the linear arrangement of these residues in the strand(s) of the amplicon. The amplicon residues comprise, adenosine (A), guanosine (G), cytidine, (C), (deoxy)thymidine (T), uracil (U), inosine (I), nitroindoles such as 5-nitroindole or 3-nitropyrrole, dP or dK (Hill et al.), an acyclic nucleoside analog containing 5-nitroindazole (Van Aerschot et al., Nucleosides and Nucleotides, 1995, 14, 1053-1056), the purine analog 1-(2-deoxy-.beta.-D-ribofuranosyl)-imidazole-4-carboxamide, 2,6-diaminopurine, 5-propynyluracil, 5-propynylcytosine, phenoxazines, including G-clamp, 5-propynyl deoxy-cytidine, deoxy-thymidine nucleotides, 5-propynylcytidine, 5-propynyluridine and mass tag modified versions thereof, including 7-deaza-2′-deoxyadenosine-5-triphosphate, 5-iodo-2′-deoxyuridine-5′-triphosphate, 5-bromo-2′-deoxyuridine-5′-triphosphate, 5-bromo-2′-deoxycytidine-5′-triphosphate, 5-iodo-2-deoxycytidine-5′-triphosphate, 5-hydroxy-2′-deoxyuridine-5′-triphosphate, 4-thiothymidine-5′-triphosphate, 5-aza-2′-deoxyuridine-5′-triphosphate, 5-fluoro-2′-deoxyuridine-5′-triphosphate methyl-2′-deoxyguanosine-5′-triphosphate, N2-methyl-2′-deoxyguanosine-5′-triphosphate, 8-oxo-2′-deoxyguanosine-5′-triphosphate or thiothymidine-5′-triphosphate. In some embodiments, the mass-modified nucleobase comprises 15.sup.N or 13.sup.0 or both 15.sup.N and 13.sup.C. Preferably, the non-natural nucleosides used herein include 5-propynyluracil, 5-propynylcytosine and inosine. Herein the base composition for an unmodified DNA amplicon is notated as A.sub.wG.sub.xC.sub.yT.sub.z, wherein w, x, y and z are each independently a whole number representing the number of said nucleoside residues in an amplicon. Base compositions for amplicons comprising modified nucleosides are similarly notated to indicate the number of said natural and modified nucleosides in an amplicon. Base compositions are calculated from a molecular mass measurement of an amplicon, as described below. The calculated base composition for any given amplicon is then compared to a database of base compositions. A match between the calculated base composition and a single database entry reveals the identity of the bioagent.

As is used herein, the term “base composition signature” refers to the base composition generated by any one particular amplicon. The base composition signature for each of one or more amplicons provides a fingerprint for identifying the bioagent(s) present in a sample.

As used herein, the term “database” is used to refer to a collection of base composition and/or molecular mass data. The base composition and/or molecular mass data in the database is indexed to bioagents and to primer pairs. The base composition data reported in the database comprises the number of each nucleoside in an amplicon that would be generated for each bioagent using each primer pair. The database can be populated by empirical data. In this aspect of populating the database, a bioagent is selected and a primer pair is used to generate an amplicon. The amplicon's molecular mass is determined using a mass spectrometer and the base composition calculated therefrom. An entry in the database is made to associate the base composition and/or molecular mass with the bioagent and the primer pair used. The database may also be populated using other databases comprising bioagent information. For example, using the GenBank database it is possible to perform electronic PCR using an electronic representation of a primer pair. This in silico method will provide the base composition for any or all selected bioagent(s) stored in the GenBank database. The information is then used to populate the base composition database as described above. A base composition database can be in silico, a written table, a reference book, a spreadsheet or any form generally amenable to databases. Preferably, it is in silico. The database can similarly be populated with molecular masses that is gathered either empirically or is calculated from other sources such as GenBank.

As used herein, the term “nucleobase” is synonymous with other terms in use in the art including “nucleotide,” “deoxynucleotide,” “nucleotide residue,” “deoxynucleotide residue,” “nucleotide triphosphate (NTP),” or deoxynucleotide triphosphate (dNTP). As is used herein, a nucleobase includes natural and modified residues, as described herein.

As used herein, a “wobble base” is a variation in a codon found at the third nucleotide position of a DNA triplet. Variations in conserved regions of sequence are often found at the third nucleotide position due to redundancy in the amino acid code.

As used herein, “housekeeping gene” refers to a gene encoding a protein or RNA involved in basic functions required for survival and reproduction of a bioagent. Housekeeping genes include, but are not limited to, genes encoding RNA or proteins involved in translation, replication, recombination and repair, transcription, nucleotide metabolism, amino acid metabolism, lipid metabolism, energy generation, uptake, secretion and the like. In some embodiments, the primers are configured to produce amplicons from within a housekeeping gene.

As used herein, a “sub-species characteristic” is a genetic characteristic that provides the means to distinguish two members of the same bioagent species. For example, one bacterial strain could be distinguished from another bacterial strain of the same species by possessing a genetic change (e.g., for example, a nucleotide deletion, addition or substitution) in one of the bacterial genes, for example, a gene conferring drug resistance or virulence.

As used herein, “triangulation identification” means the employment of more than one primer pair to generate a corresponding amplicon for identification of a bioagent. The more than one primer pair can be used in individual wells or in a multiplex PCR assay. Alternatively, PCR reaction may be carried out in single wells comprising a different primer pair in each well. Following amplification, the amplicons are pooled into a single well or container which is then subjected to molecular mass analysis. The combination of pooled amplicons can be chosen such that the expected ranges of molecular masses of individual amplicons are not overlapping and thus will not complicate identification of signals. Triangulation works as a process of elimination, wherein a first primer pair identifies that an unknown bioagent may be one of a group of bioagents. Subsequent primer pairs are used in triangulation identification to further refine the identity of the bioagent amongst the subset of possibilities generated with the earlier primer pair. Triangulation identification is complete when the identity of the bioagent is determined. The triangulation identification process is also used to reduce false negative and false positive signals, and enable reconstruction of the origin of hybrid or otherwise engineered bioagents. For example, identification of the three part toxin genes typical of B. anthracis (Bowen et al., J. Appl. Microbiol., 1999, 87, 270-278) in the absence of the expected signatures from the B. anthracis genome would suggest a genetic engineering event. In one example, a first pair of primers might determine that a given bioagent is a member of the Staphylococcus genus. A second primer pair may identify the bioagent as a member of the Staphylococcus aureus species, while a third primer may identify a sub-species characteristic of the bioagent, for example, resistance to a particular antibiotic or strain information.

As used herein, the term “triangulation genotyping analysis primer pair” is a primer pair configured to produce bioagent identifying amplicons for determining species types in a triangulation genotyping analysis.

As is used herein, the term “single primer pair identification” means that one or more bioagents can be identified using a single primer pair. A base composition signature for an amplicon may singly identify one or more bioagents.

As used herein, the term “etiology” refers to the causes or origins, of diseases or abnormal physiological conditions.

As used herein, the term “concurrently amplifying” used with respect to more than one amplification reaction refers to the act of simultaneously amplifying more than one nucleic acid in a single reaction mixture.

The term “duplex” refers to the state of nucleic acids in which the base portions of the nucleotides on one strand are bound through hydrogen bonding the their complementary bases arrayed on a second strand. The condition of being in a duplex form reflects on the state of the bases of a nucleic acid. By virtue of base pairing, the strands of nucleic acid also generally assume the tertiary structure of a double helix, having a major and a minor groove. The assumption of the helical form is implicit in the act of becoming duplexed.

The term “gene” refers to a DNA sequence that comprises control and coding sequences necessary for the production of an RNA having a non-coding function (e.g., a ribosomal or transfer RNA), a polypeptide or a precursor. The RNA or polypeptide can be encoded by a full length coding sequence or by any portion of the coding sequence so long as the desired activity or function is retained.

The terms “homology,” “homologous” and “sequence identity” refer to a degree of identity. There may be partial homology or complete homology. A partially homologous sequence is one that is less than 100% identical to another sequence. Determination of sequence identity is described in the following example: a primer 20 nucleobases in length which is otherwise identical to another 20 nucleobase primer but having two non-identical residues has 18 of 20 identical residues ( 18/20=0.9 or 90% sequence identity). In another example, a primer 15 nucleobases in length having all residues identical to a 15 nucleobase segment of a primer 20 nucleobases in length would have 15/20=0.75 or 75% sequence identity with the 20 nucleobase primer. Herein, sequence identity is meant to be properly determined when the query sequence and the subject sequence are both described and aligned in the 5′ to 3′ direction. Sequence alignment algorithms such as BLAST, will return results in two different alignment orientations. In the Plus/Plus orientation, both the query sequence and the subject sequence are aligned in the 5′ to 3′ direction. On the other hand, in the Plus/Minus orientation, the query sequence is in the 5′ to 3′ direction while the subject sequence is in the 3′ to 5′ direction. It should be understood that with respect to the primers provided herein, sequence identity is properly determined when the alignment is designated as Plus/Plus. Sequence identity may also encompass alternate or modified nucleobases that perform in a functionally similar manner to the regular nucleobases adenine, thymine, guanine and cytosine with respect to hybridization and primer extension in amplification reactions. In a non-limiting example, if the 5-propynyl pyrimidines propyne C and/or propyne T replace one or more C or T residues in one primer which is otherwise identical to another primer in sequence and length, the two primers will have 100% sequence identity with each other. In another non-limiting example, Inosine (I) may be used as a replacement for G or T and effectively hybridize to C, A or U (uracil). Thus, if inosine replaces one or more C, A or U residues in one primer which is otherwise identical to another primer in sequence and length, the two primers will have 100% sequence identity with each other. Other such modified or universal bases may exist which would perform in a functionally similar manner for hybridization and amplification reactions and will be understood to fall within this definition of sequence identity.

As used herein, the term “hybridization” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, and the Tₘ of the formed hybrid. “Hybridization” methods involve the annealing of one nucleic acid to another, complementary nucleic acid, i.e., a nucleic acid having a complementary nucleotide sequence. The ability of two polymers of nucleic acid containing complementary sequences to find each other and anneal through base pairing interaction is a well-recognized phenomenon. The initial observations of the “hybridization” process by Marmur and Lane, Proc. Natl. Acad. Sci. USA 46:453 (1960) and Doty et al., Proc. Natl. Acad. Sci. USA 46:461 (1960) have been followed by the refinement of this process into an essential tool of modem biology.

As used herein, the term “polymerase chain reaction” (“PCR”) refers to the method of K. B. Mullis U.S. Pat. Nos. 4,683,195, 4,683,202, and 4,965,188, hereby incorporated by reference, that describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.” With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of 32P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

The term “in silico” refers to processes taking place via computer calculations. For example, electronic PCR (ePCR) is a process analogous to ordinary PCR except that it is carried out using nucleic acid sequences and primer pair sequences stored on a computer formatted medium.

The term “polymerase” refers to an enzyme having the ability to synthesize a complementary strand of nucleic acid from a starting template nucleic acid strand and free dNTPs.

The term “polymerization means” or “polymerization agent” refers to any agent capable of facilitating the addition of nucleoside triphosphates to an oligonucleotide. Preferred polymerization means comprise DNA and RNA polymerases.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “mass-modifying tag” refers to any modification to a given nucleotide which results in an increase in mass relative to the analogous non-mass modified nucleotide. Mass-modifying tags can include heavy isotopes of one or more elements included in the nucleotide such as carbon-13 for example. Other possible modifications include addition of substituents such as iodine or bromine at the 5 position of the nucleobase for example.

The term “microorganism” as used herein means an organism too small to be observed with the unaided eye and includes, but is not limited to bacteria, virus, protozoans, fungi; and ciliates.

The term “multi-drug resistant” or “multiple-drug resistant” refers to a microorganism which is resistant to more than one of the antibiotics or antimicrobial agents used in the treatment of said microorganism.

The term “non-template tag” refers to a stretch of at least three guanine or cytosine nucleobases of a primer used to produce a bioagent identifying amplicon which are not complementary to the template. A non-template tag is incorporated into a primer for the purpose of increasing the primer-duplex stability of later cycles of amplification by incorporation of extra G-C pairs which each have one additional hydrogen bond relative to an A-T pair.

The term “nucleic acid sequence” as used herein refers to the linear composition of the nucleic acid residues A, T, C, G, U, or any modifications thereof, within an oligonucleotide, nucleotide or polynucleotide, and fragments or portions thereof, and to DNA or RNA of genomic or synthetic origin which may be single or double stranded, and represent the sense or antisense strand

As used herein, the term “nucleobase” is synonymous with other terms in use in the art including “nucleotide,” “deoxynucleotide,” “nucleotide residue,” “deoxynucleotide residue,” “nucleotide triphosphate (NTP),” or deoxynucleotide triphosphate (dNTP).

The term “nucleotide analog” as used herein refers to modified or non-naturally occurring nucleotides such as 5-propynyl pyrimidines (i.e., 5-propynyl-dTTP and 5-propynyl-dTCP), 7-deaza purines (i.e., 7-deaza-dATP and 7-deaza-dGTP). Nucleotide analogs include base analogs and comprise modified forms of deoxyribonucleotides as well as ribonucleotides.

The term “oligonucleotide” as used herein is defined as a molecule comprising two or more deoxyribonucleotides or ribonucleotides, preferably at least 5 nucleotides, more preferably at least about 13 to 35 nucleotides. The exact size will depend on many factors, which in turn depend on the ultimate function or use of the oligonucleotide. The oligonucleotide may be generated in any manner, including chemical synthesis, DNA replication, reverse transcription, PCR, or a combination thereof. Because mononucleotides are reacted to make oligonucleotides in a manner such that the 5′ phosphate of one mononucleotide pentose ring is attached to the 3′ oxygen of its neighbor in one direction via a phosphodiester linkage, an end of an oligonucleotide is referred to as the “5′-end” if its 5′ phosphate is not linked to the 3′ oxygen of a mononucleotide pentose ring and as the “3′-end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends. A first region along a nucleic acid strand is said to be upstream of another region if the 3′ end of the first region is before the 5′ end of the second region when moving along a strand of nucleic acid in a 5′ to 3′ direction. All oligonucleotide primers disclosed herein are understood to be presented in the 5′ to 3′ direction when reading left to right. When two different, non-overlapping oligonucleotides anneal to different regions of the same linear complementary nucleic acid sequence, and the 3′ end of one oligonucleotide points towards the 5′ end of the other, the former may be called the “upstream” oligonucleotide and the latter the “downstream” oligonucleotide. Similarly, when two overlapping oligonucleotides are hybridized to the same linear complementary nucleic acid sequence, with the first oligonucleotide positioned such that its 5′ end is upstream of the 5′ end of the second oligonucleotide, and the 3′ end of the first oligonucleotide is upstream of the 3′ end of the second oligonucleotide, the first oligonucleotide may be called the “upstream” oligonucleotide and the second oligonucleotide may be called the “downstream” oligonucleotide.

As used herein, the terms “purified” or “substantially purified” refer to molecules, either nucleic or amino acid sequences, that are removed from their natural environment, isolated or separated, and are at least 60% free, preferably 75% free, and most preferably 90% free from other components with which they are naturally associated. An “isolated polynucleotide” or “isolated oligonucleotide” is therefore a substantially purified polynucleotide.

The term “reverse transcriptase” refers to an enzyme having the ability to transcribe DNA from an RNA template. This enzymatic activity is known as reverse transcriptase activity. Reverse transcriptase activity is desirable in order to obtain DNA from RNA viruses which can then be amplified and analyzed by the methods provided herein.

The term “ribosomal RNA” or “rRNA” refers to the primary ribonucleic acid constituent of ribosomes. Ribosomes are the protein-manufacturing organelles of cells and exist in the cytoplasm. Ribosomal RNAs are transcribed from the DNA genes encoding them.

The term “sample” in the present specification and claims is used in its broadest sense. On the one hand it is meant to include a specimen or culture (e.g., microbiological cultures). On the other hand, it is meant to include both biological and environmental samples. A sample may include a specimen of synthetic origin. Biological samples may be animal, including human, fluid, solid (e.g., stool) or tissue, as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagamorphs, rodents, etc. Environmental samples include environmental material such as surface matter, soil, water, air and industrial samples, as well as samples obtained from food and dairy processing instruments, apparatus, equipment, utensils, disposable and non-disposable items. These examples are not to be construed as limiting the sample types applicable to embodiments provided herein. The term “source of target nucleic acid” refers to any sample that contains nucleic acids (RNA or DNA). Particularly preferred sources of target nucleic acids are biological samples including, but not limited to blood, saliva, cerebral spinal fluid, pleural fluid, milk, lymph, sputum and semen.

As used herein, the term “sample template” refers to nucleic acid originating from a sample that is analyzed for the presence of “target” (defined below). In contrast, “background template” is used in reference to nucleic acid other than sample template that may or may not be present in a sample. Background template is often a contaminant. It may be the result of carryover, or it may be due to the presence of nucleic acid contaminants sought to be purified away from the sample. For example, nucleic acids from organisms other than those to be detected may be present as background in a test sample.

A “segment” is defined herein as a region of nucleic acid within a target sequence.

As used herein, the term ““sequence alignment”” refers to a listing of multiple DNA or amino acid sequences and aligns them to highlight their similarities. The listings can be made using bioinformatics computer programs.

As used herein, the term “target” is used in a broad sense to indicate the gene or genomic region being amplified by the primers. Because a given primer pair provided herein is configured to generate a plurality of amplification products (depending on the bioagent being analyzed), multiple amplification products from different specific nucleic acid sequences may be obtained. Thus, the term “target” is not used to refer to a single specific nucleic acid sequence. The “target” is sought to be sorted out from other nucleic acid sequences and contains a sequence that has at least partial complementarity with an oligonucleotide primer. The target nucleic acid may comprise single- or double-stranded DNA or RNA. Primers herein can be targeted to, or configured to hybridize within portions, segments, or regions of nucleic acids. These terms are used when referring to specific regions of nucleic acid sequences used in primer design.

The term “template” refers to a strand of nucleic acid on which a complementary copy is built from nucleoside triphosphates through the activity of a template-dependent nucleic acid polymerase. Within a duplex the template strand is, by convention, depicted and described as the “bottom” strand. Similarly, the non-template strand is often depicted and described as the “top” strand.

As used herein, the term “T.sub.m” is used in reference to the “melting temperature.” The melting temperature is the temperature at which a population of double-stranded nucleic acid molecules becomes half dissociated into single strands. Several equations for calculating the T.sub.m of nucleic acids are well known in the art. As indicated by standard references, a simple estimate of the T.sub.m value may be calculated by the equation: T.sub.m=81.5+0.41(% G+C), when a nucleic acid is in aqueous solution at 1 M NaCl (see e.g., Anderson and Young, Quantitative Filter Hybridization, in Nucleic Acid Hybridization (1985). Other references (e.g., Allawi, H. T. & SantaLucia, J., Jr. Thermodynamics and NMR of internal G.T mismatches in DNA. Biochemistry 36, 10581-94 (1997) include more sophisticated computations which take structural and environmental, as well as sequence characteristics into account for the calculation of T.sub.m.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified”, “mutant” or “polymorphic” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

Provided herein are methods for detection and identification of bioagents in an unbiased manner using bioagent identifying amplicons. In one aspect, the methods are for detection and identification of population genotype for a population of bioagents. Primers are selected to hybridize to conserved sequence regions of nucleic acids derived from a bioagent and which bracket (flank) variable sequence regions to yield a bioagent identifying amplicon which can be amplified and which is amenable to molecular mass determination. The molecular mass is converted to a base composition, which indicates the number of each nucleotide in the amplicon. The molecular mass or corresponding base composition signature of the amplicon is then queried against a database of molecular masses or base composition signatures indexed to bioagents and to the primer pair used to generate the amplicon. A match of the measured base composition to a database entry base composition associates the sample bioagent to an indexed bioagent in the database. Thus, the identity of the unknown bioagent or population of bioagents is determined. Prior knowledge of the unknown bioagent or population of bioagents is not necessary. In some instances, the measured base composition associates with more than one database entry base composition. Thus, a second/subsequent primer pair is used to generate an amplicon, and its measured base composition is similarly compared to the database to determine its identity in triangulation identification. Furthermore, the method can be applied to rapid parallel multiplex analyses, the results of which can be employed in a triangulation identification strategy. The present method provides rapid throughput and does not require nucleic acid sequencing of the amplified target sequence for bioagent detection and identification.

Calculation of base composition from a mass spectrometer generated molecular mass becomes increasingly more complex as the length of the amplicon increases. For amplicons comprising unmodified nucleic acid, the upper length as a practical length limit is about 200 consecutive nucleobases. Incorporating modified nucleotides into the amplicon can allow for an increase in this upper limit. In one embodiment, the amplicons generated using any single primer pair will provide sufficient base composition information to allow for identification of at least one bioagent at the family, genus, species or subspecies level. Alternatively, amplicons greater than 200 nucleobases can be generated and then digested to form two or more fragments that are less than 200 nucleobases. Analysis of one or more of the fragments will provide sufficient base composition information to allow for identification of at least one bioagent.

Preferably, amplicons comprise from about 45 to about 200 consecutive nucleobases (i.e., from about 45 to about 200 linked nucleosides). One of ordinary skill in the art will appreciate that this range expressly embodies compounds of 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, and 200 nucleobases in length. One ordinarily skilled in the art will further appreciate that the above range is not an absolute limit to the length of an amplicon, but instead represents a preferred length range. Amplicons lengths falling outside of this range are also included herein so long as the amplicon is amenable to calculation of a base composition signature as herein described.

In some embodiments, bioagent identifying amplicons amenable to molecular mass determination that are produced by the primers described herein are either of a length, size and/or mass compatible with the particular mode of molecular mass determination or compatible with a means of providing a predictable fragmentation pattern in order to obtain predictable fragments of a length compatible with the particular mode of molecular mass determination. Such means of providing a predictable fragmentation pattern of an amplicon include, but are not limited to, cleavage with restriction enzymes or cleavage primers, for example. Thus, in some embodiments, bioagent identifying amplicons are larger than 200 nucleobases and are amenable to molecular mass determination following restriction digestion. Methods of using restriction enzymes and cleavage primers are well known to those with ordinary skill in the art.

In some embodiments, amplicons corresponding to bioagent identifying amplicons are obtained using the polymerase chain reaction (PCR) which is a routine method to those with ordinary skill in the molecular biology arts. Other amplification methods may be used such as ligase chain reaction (LCR), low-stringency single primer PCR, and multiple strand displacement amplification (MDA). These methods are also known to those with ordinary skill. (Michael, S F., Biotechniques (1994), 16:411-412 and Dean et al., Proc. Natl. Acad. Sci. U.S.A. (2002), 99, 5261-5266). In some embodiments, the amplification is carried out in a multiplex assay, a PCR amplification reaction where more than one primer pair is included in the reaction pool allowing two or more different DNA targets to be amplified in a single tube or well.

Unlike bacterial genomes, which exhibit conservation of numerous genes (i.e. housekeeping genes) across all organisms, viruses do not share a gene that is essential and conserved among all virus families. Therefore, viral identification is achieved within smaller groups of related viruses, such as members of a particular virus family or genus. For example, RNA-dependent RNA polymerase is present in all single-stranded RNA viruses and can be used for broad priming as well as resolution within the virus family.

In some embodiments, at least one bacterial nucleic acid segment is amplified in the process of identifying the bacterial bioagent. Thus, the nucleic acid segments that can be amplified by the primers disclosed herein and that provide enough variability to distinguish each individual bioagent and whose molecular masses are amenable to molecular mass determination are herein described as bioagent identifying amplicons.

In some embodiments, identification of bioagents is accomplished at different levels using primers suited to resolution of each individual level of identification. Broad range survey primers are configured with the objective of identifying a bioagent as a member of a particular division (e.g., an order, family, genus or other such grouping of bioagents above the species level of bioagents). In some embodiments, broad range survey intelligent primers are capable of identification of bioagents at the species or sub-species level. Examples of broad range survey primers include, but are not limited to: primer pair numbers: 346 (SEQ ID NOs: 202:1110), 347 (SEQ ID NOs: 560:1278), 348 SEQ ID NOs: 706:895), and 361 (SEQ ID NOs: 697:1398) which target DNA encoding 16S rRNA, and primer pair numbers 349 (SEQ ID NOs: 401:1156) and 360 (SEQ ID NOs: 409:1434) which target DNA encoding 23S rRNA.

In some embodiments, drill-down primers are configured with the objective of identifying a bioagent at the sub-species level (including strains, subtypes, variants and isolates) based on sub-species characteristics which may, for example, include single nucleotide polymorphisms (SNPs), variable number tandem repeats (VNTRs), deletions, drug resistance mutations or any other modification of a nucleic acid sequence of a bioagent relative to other members of a species having different sub-species characteristics. Drill-down intelligent primers are not always required for identification at the sub-species level because broad range survey intelligent primers may, in some cases provide sufficient identification resolution to accomplishing this identification objective. Examples of drill-down primers include, but are not limited to: confirmation primer pairs such as primer pair numbers 351 (SEQ ID NOs: 355:1423) and 353 (SEQ ID NOs: 220:1394), which target the pX01 virulence plasmid of Bacillus anthracis. Other examples of drill-down primer pairs are found in sets of triangulation genotyping primer pairs such as, for example, the primer pair number 2146 (SEQ ID NOs: 437:1137) which targets the arcC gene (encoding carmabate kinase) and is included in an 8 primer pair panel or kit for use in genotyping Staphylococcus aureus, or in other panels or kits of primer pairs used for determining drug-resistant bacterial strains, such as, for example, primer pair number 2095 (SEQ ID NOs: 456:1261) which targets the pv-luk gene (encoding Panton-Valentine leukocidin) and is included in an 8 primer pair panel or kit for use in identification of drug resistant strains of Staphylococcus aureus.

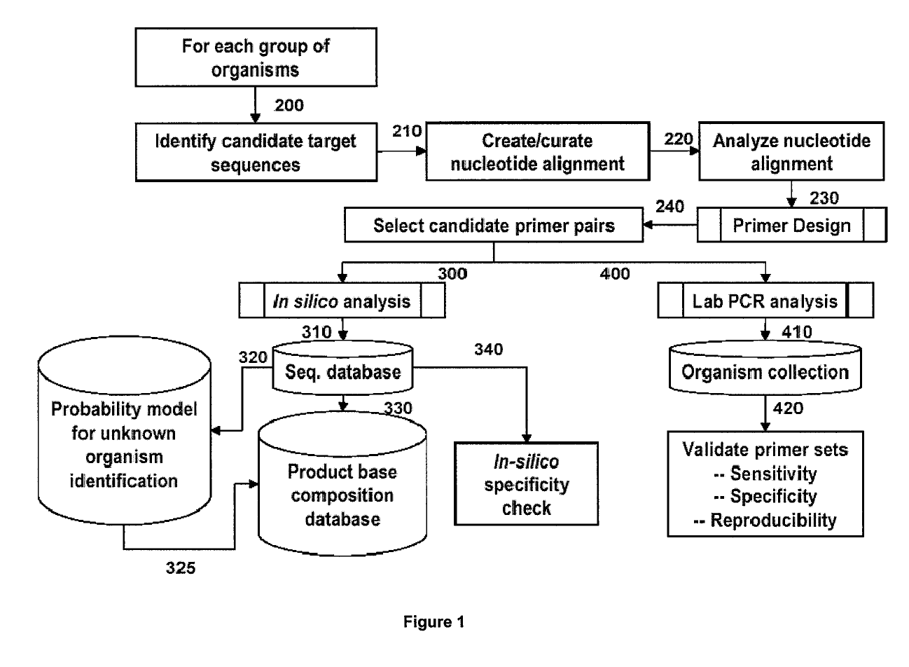

A representative process flow diagram used for primer selection and validation process is outlined in FIG. 1. For each group of organisms, candidate target sequences are identified (200) from which nucleotide alignments are created (210) and analyzed (220). Primers are then configured by selecting appropriate priming regions (230) to facilitate the selection of candidate primer pairs (240). The primer pairs are then subjected to in silico analysis by electronic PCR (ePCR) (300) wherein bioagent identifying amplicons are obtained from sequence databases such as GenBank or other sequence collections (310) and checked for specificity in silico (320). Bioagent identifying amplicons obtained from GenBank sequences (310) can also be analyzed by a probability model which predicts the capability of a given amplicon to identify unknown bioagents such that the base compositions of amplicons with favorable probability scores are then stored in a base composition database (325). Alternatively, base compositions of the bioagent identifying amplicons obtained from the primers and GenBank sequences can be directly entered into the base composition database (330). Candidate primer pairs (240) are validated by testing their ability to hybridize to target nucleic acid by an in vitro amplification by a method such as PCR analysis (400) of nucleic acid from a collection of organisms (410). Amplification products thus obtained are analyzed by gel electrophoresis or by mass spectrometry to confirm the sensitivity, specificity and reproducibility of the primers used to obtain the amplification products (420).

Many of the important pathogens, including the organisms of greatest concern as biowarfare agents, have been completely sequenced. This effort has greatly facilitated the design of primers for the detection of unknown bioagents. The combination of broad-range priming with division-wide and drill-down priming has been used very successfully in several applications of the technology, including environmental surveillance for biowarfare threat agents and clinical sample analysis for medically important pathogens.

Synthesis of primers is well known and routine in the art. The primers may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed.

In some embodiments, the oligonucleotide primers are broad range survey primers which hybridize to conserved regions of nucleic acid encoding the hexon gene of all (or between 80% and 100%, between 85% and 100%, between 90% and 100% or between 95% and 100%) known bacteria and produce bacterial bioagent identifying amplicons.

In some cases, the molecular mass or base composition of a bacterial bioagent identifying amplicon defined by a broad range survey primer pair does not provide enough resolution to unambiguously identify a bacterial bioagent at or below the species level. These cases benefit from further analysis of one or more bacterial bioagent identifying amplicons generated from at least one additional broad range survey primer pair or from at least one additional division-wide primer pair. The employment of more than one bioagent identifying amplicon for identification of a bioagent is herein referred to as triangulation identification.

In other embodiments, the oligonucleotide primers are division-wide primers which hybridize to nucleic acid encoding genes of species within a genus of bacteria. In other embodiments, the oligonucleotide primers are drill-down primers which enable the identification of sub-species characteristics. Drill down primers provide the functionality of producing bioagent identifying amplicons for drill-down analyses such as strain typing when contacted with nucleic acid under amplification conditions. Identification of such sub-species characteristics is often critical for determining proper clinical treatment of viral infections. In some embodiments, sub-species characteristics are identified using only broad range survey primers and division-wide and drill-down primers are not used.

In some embodiments, the primers used for amplification hybridize to and amplify genomic DNA, and DNA of bacterial plasmids.

In some embodiments, various computer software programs may be used to aid in design of primers for amplification reactions such as Primer Premier 5 (Premier Biosoft, Palo Alto, Calif.) or OLIGO Primer Analysis Software (Molecular Biology Insights, Cascade, Colo.). These programs allow the user to input desired hybridization conditions such as melting temperature of a primer-template duplex for example. In some embodiments, an in silico PCR search algorithm, such as (ePCR) is used to analyze primer specificity across a plurality of template sequences which can be readily obtained from public sequence databases such as GenBank for example. An existing RNA structure search algorithm (Macke et al., Nucl. Acids Res., 2001, 29, 4724-4735, which is incorporated herein by reference in its entirety) has been modified to include PCR parameters such as hybridization conditions, mismatches, and thermodynamic calculations (SantaLucia, Proc. Natl. Acad. Sci. U.S.A., 1998, 95, 1460-1465, which is incorporated herein by reference in its entirety). This also provides information on primer specificity of the selected primer pairs. In some embodiments, the hybridization conditions applied to the algorithm can limit the results of primer specificity obtained from the algorithm. In some embodiments, the melting temperature threshold for the primer template duplex is specified to be 35° C. or a higher temperature. In some embodiments the number of acceptable mismatches is specified to be seven mismatches or less. In some embodiments, the buffer components and concentrations and primer concentrations may be specified and incorporated into the algorithm, for example, an appropriate primer concentration is about 250 nM and appropriate buffer components are 50 mM sodium or potassium and 1.5 mM Mg.sup.2+.

One with ordinary skill in the art of design of amplification primers will recognize that a given primer need not hybridize with 100% complementarity in order to effectively prime the synthesis of a complementary nucleic acid strand in an amplification reaction. Moreover, a primer may hybridize over one or more segments such that intervening or adjacent segments are not involved in the hybridization event. (e.g., for example, a loop structure or a hairpin structure). The primers provided herein may comprise at least 70%, at least 75%, at least 80%, at least 85%, at least 90%, at least 95% or at least 99% sequence identity with any of the primers listed in Table 2. Thus, in some embodiments, an extent of variation of 70% to 100%, or any range therewithin, of the sequence identity is possible relative to the specific primer sequences disclosed herein. Determination of sequence identity is described in the following example: a primer 20 nucleobases in length which is identical to another 20 nucleobase primer having two non-identical residues has 18 of 20 identical residues ( 18/20=0.9 or 90% sequence identity). In another example, a primer 15 nucleobases in length having all residues identical to a 15 nucleobase segment of primer 20 nucleobases in length would have 15/20=0.75 or 75% sequence identity with the 20 nucleobase primer. Similarly, either or both of the primers of the primer pairs provided herein may comprise 0-10 nucleobase deletions, additions, and/or substitutions relative to any of the primers listed in Table 2, or elsewhere herein. In other words, either or both of the primers may comprise 0, 1, 2, 3, 4, 5, 6, 7, 8, 9, or 10 nucleobase deletions, 0, 1, 2, 3, 4, 5, 6, 7, 8, 9, or 10 nucleobase additions, 0, 1, 2, 3, 4, 5, 6, 7, 8, 9, or 10 nucleobase substitutions relative to the sequences of any of the primers disclosed herein. In one aspect, the primers comprise the sequence of any of the primers listed in Table 2 with the T modification removed from the 5′ terminus. In one aspect, the primers comprise the sequence of any of the primers listed in Table 2 with the T modification removed from the 5′ terminus and comprising 0-10 nucleobase deletions, additions, and/or substitutions.

Percent homology, sequence identity or complementarity, can be determined by, for example, the Gap program (Wisconsin Sequence Analysis Package, Version 8 for UNIX, Genetics Computer Group, University Research Park, Madison Wis.), using default settings, which uses the algorithm of Smith and Waterman (Adv. Appl. Math., 1981, 2, 482-489). In some embodiments, complementarity of primers with respect to the conserved priming regions of viral nucleic acid is between about 70% and about 75% 80%. In other embodiments, homology, sequence identity or complementarity, is between about 75% and about 80%. In yet other embodiments, homology, sequence identity or complementarity, is at least 85%, at least 90%, at least 92%, at least 94%, at least 95%, at least 96%, at least 97%, at least 98%, at least 99% or is 100%. In some embodiments, the primers described herein comprise at least 70%, at least 75%, at least 80%, at least 85%, at least 90%, at least 92%, at least 94%, at least 95%, at least 96%, at least 98%, or at least 99%, or 100% (or any range therewithin) sequence identity with the primer sequences specifically disclosed herein.

One with ordinary skill is able to calculate percent sequence identity or percent sequence homology and able to determine, without undue experimentation, the effects of variation of primer sequence identity on the function of the primer in its role in priming synthesis of a complementary strand of nucleic acid for production of an amplification product of a corresponding bioagent identifying amplicon.

In some embodiments, the oligonucleotide primers are 13 to 35 nucleobases in length (13 to 35 linked nucleotide residues). In these embodiments, the primers are at least 13 nucleobases in length, and less than 36 nucleobases in length. These embodiments comprise oligonucleotide primers 13, 14, 15, 16, 17, 18, 19, 20, 21, 22, 23, 24, 25, 26, 27, 28, 29, 30, 31, 32, 33, 34 or 35 nucleobases in length, or any range therewithin. Herein is contemplated using both longer and shorter primers. Furthermore, the primers may also be linked to one or more other desired moieties, including, but not limited to, affinity groups, ligands, regions of nucleic acid that are not complementary to the nucleic acid to be amplified, labels, etc. Primers may also form hairpin structures. For example, hairpin primers may be used to amplify short target nucleic acid molecules. The presence of the hairpin may stabilize the amplification complex (see e.g., TAQMAN MicroRNA Assays, Applied Biosystems, Foster City, Calif.).

In some embodiments, any oligonucleotide primer pair may have one or both primers with less then 70% sequence homology with a corresponding member of any of the primer pairs of Table 2 if the primer pair has the capability of producing an amplification product corresponding to a bioagent identifying amplicon. In other embodiments, any oligonucleotide primer pair may have one or both primers with a length greater than 35 nucleobases if the primer pair has the capability of producing an amplification product corresponding to a bioagent identifying amplicon.

In some embodiments, the function of a given primer may be substituted by a combination of two or more primers segments that hybridize adjacent to each other or that are linked by a nucleic acid loop structure or linker which allows a polymerase to extend the two or more primers in an amplification reaction.

In some embodiments, the primer pairs used for obtaining bioagent identifying amplicons are the primer pairs of Table 2. In other embodiments, other combinations of primer pairs are possible by combining certain members of the forward primers with certain members of the reverse primers. An example can be seen in Table 2 for two primer pair combinations of forward primer 16S_EC_{—}789_{—}810_F (SEQ ID NO: 206), with the reverse primers 16S_EC_{—}880_{—}894_R (SEQ ID NO: 796), or 16S_EC_{—}882_{—}899_R or (SEQ ID NO: 818). Arriving at a favorable alternate combination of primers in a primer pair depends upon the properties of the primer pair, most notably the size of the bioagent identifying amplicon that would be produced by the primer pair, which preferably is between about 45 to about 150 nucleobases in length. Alternatively, a bioagent identifying amplicon longer than 150 nucleobases in length could be cleaved into smaller segments by cleavage reagents such as chemical reagents, or restriction enzymes, for example.

In some embodiments, the primers are configured to amplify nucleic acid of a bioagent to produce amplification products that can be measured by mass spectrometry and from whose molecular masses candidate base compositions can be readily calculated.

In some embodiments, any given primer comprises a modification comprising the addition of a non-templated T residue to the 5′ end of the primer (i.e., the added T residue does not necessarily hybridize to the nucleic acid being amplified). The addition of a non-templated T residue has an effect of minimizing the addition of non-templated adenosine residues as a result of the non-specific enzyme activity of Taq polymerase (Magnuson et al., Biotechniques, 1996, 21, 700-709), an occurrence which may lead to ambiguous results arising from molecular mass analysis.

In some embodiments, primers may contain one or more universal bases. Because any variation (due to codon wobble in the third position) in the conserved regions among species is likely to occur in the third position of a DNA (or RNA) triplet, oligonucleotide primers can be configured such that the nucleotide corresponding to this position is a base which can bind to more than one nucleotide, referred to herein as a “universal nucleobase.” For example, under this “wobble” pairing, inosine (I) binds to U, C or A; guanine (G) binds to U or C, and uridine (U) binds to U or C. Other examples of universal nucleobases include nitroindoles such as 5-nitroindole or 3-nitropyrrole (Loakes et al., Nucleosides and Nucleotides, 1995, 14, 1001-1003), the degenerate nucleotides dP or dK (Hill et al.), an acyclic nucleoside analog containing 5-nitroindazole (Van Aerschot et al., Nucleosides and Nucleotides, 1995, 14, 1053-1056) or the purine analog 1-(2-deoxy-beta-D-ribofuranosyl)-imidazole-4-carboxamide (Sala et al., Nucl. Acids Res., 1996, 24, 3302-3306).

In some embodiments, to compensate for the somewhat weaker binding by the wobble base, the oligonucleotide primers are configured such that the first and second positions of each triplet are occupied by nucleotide analogs that bind with greater affinity than the unmodified nucleotide. Examples of these analogs include, but are not limited to, 2,6-diaminopurine which binds to thymine, 5-propynyluracil (also known as propynylated thymine) which binds to adenine and 5-propynylcytosine and phenoxazines, including G-clamp, which binds to G. Propynylated pyrimidines are described in U.S. Pat. Nos. 5,645,985, 5,830,653 and 5,484,908, each of which is commonly owned and incorporated herein by reference in its entirety. Propynylated primers are described in U.S Pre-Grant Publication No. 2003-0170682, which is also commonly owned and incorporated herein by reference in its entirety. Phenoxazines are described in U.S. Pat. Nos. 5,502,177, 5,763,588, and 6,005,096, each of which is incorporated herein by reference in its entirety. G-clamps are described in U.S. Pat. Nos. 6,007,992 and 6,028,183, each of which is incorporated herein by reference in its entirety.

In some embodiments, primer hybridization is enhanced using primers containing 5-propynyl deoxy-cytidine and deoxy-thymidine nucleotides. These modified primers offer increased affinity and base pairing selectivity.

In some embodiments, non-template primer tags are used to increase the melting temperature (T.sub.m) of a primer-template duplex in order to improve amplification efficiency. A non-template tag is at least three consecutive A or T nucleotide residues on a primer which are not complementary to the template. In any given non-template tag, A can be replaced by C or G and T can also be replaced by C or G. Although Watson-Crick hybridization is not expected to occur for a non-template tag relative to the template, the extra hydrogen bond in a G-C pair relative to an A-T pair confers increased stability of the primer-template duplex and improves amplification efficiency for subsequent cycles of amplification when the primers hybridize to strands synthesized in previous cycles.

In other embodiments, propynylated tags may be used in a manner similar to that of the non-template tag, wherein two or more 5-propynylcytidine or 5-propynyluridine residues replace template matching residues on a primer. In other embodiments, a primer contains a modified internucleoside linkage such as a phosphorothioate linkage, for example.

In some embodiments, the primers comprise mass-modifying tags. Reducing the total number of possible base compositions of a nucleic acid of specific molecular weight provides a means of avoiding a persistent source of ambiguity in determination of base composition of amplification products. Addition of mass-modifying tags to certain nucleobases of a given primer will result in simplification of de novo determination of base composition of a given bioagent identifying amplicon from its molecular mass.

In some embodiments, the mass modified nucleobase comprises one or more of the following: for example, 7-deaza-2′-deoxyadenosine-5-triphosphate, 5-iodo-2′-deoxyuridine-5′-triphosphate, 5-bromo-2′-deoxyuridine-5′-triphosphate, 5-bromo-2′-deoxycytidine-5′-triphosphate, 5-iodo-2′-deoxycytidine-5′-triphosphate, 5-hydroxy-2′-deoxyuridine-5′-triphosphate, 4-thiothymidine-5′-triphosphate, 5-aza-2′-deoxyuridine-5′-triphosphate, 5-fluoro-2′-deoxyuridine-5′-triphosphate, O6-methyl-2′-deoxyguanosine-5′-triphosphate, N2-methyl-2′-deoxyguanosine-5′-triphosphate, 8-oxo-2′-deoxyguanosine-5′-triphosphate or thiothymidine-5′-triphosphate. In some embodiments, the mass-modified nucleobase comprises ¹⁵N or ¹³C or both ¹⁵N and ¹³C.

In some embodiments, multiplex amplification is performed where multiple bioagent identifying amplicons are amplified with a plurality of primer pairs. The advantages of multiplexing are that fewer reaction containers (for example, wells of a 96- or 384-well plate) are needed for each molecular mass measurement, providing time, resource and cost savings because additional bioagent identification data can be obtained within a single analysis. Multiplex amplification methods are well known to those with ordinary skill and can be developed without undue experimentation. However, in some embodiments, one useful and non-obvious step in selecting a plurality candidate bioagent identifying amplicons for multiplex amplification is to ensure that each strand of each amplification product will be sufficiently different in molecular mass that mass spectral signals will not overlap and lead to ambiguous analysis results. In some embodiments, a 10 Da difference in mass of two strands of one or more amplification products is sufficient to avoid overlap of mass spectral peaks.

In some embodiments, as an alternative to multiplex amplification, single amplification reactions can be pooled before analysis by mass spectrometry. In these embodiments, as for multiplex amplification embodiments, it is useful to select a plurality of candidate bioagent identifying amplicons to ensure that each strand of each amplification product will be sufficiently different in molecular mass that mass spectral signals will not overlap and lead to ambiguous analysis results.

In some embodiments, the molecular mass of a given bioagent identifying amplicon is determined by mass spectrometry. Mass spectrometry has several advantages, not the least of which is high bandwidth characterized by the ability to separate (and isolate) many molecular peaks across a broad range of mass to charge ratio (m/z). Thus mass spectrometry is intrinsically a parallel detection scheme without the need for radioactive or fluorescent labels, since every amplification product is identified by its molecular mass. The current state of the art in mass spectrometry is such that less than femtomole quantities of material can be readily analyzed to afford information about the molecular contents of the sample. An accurate assessment of the molecular mass of the material can be quickly obtained, irrespective of whether the molecular weight of the sample is several hundred, or in excess of one hundred thousand atomic mass units (amu) or Daltons.

In some embodiments, intact molecular ions are generated from amplification products using one of a variety of ionization techniques to convert the sample to gas phase. These ionization methods include, but are not limited to, electrospray ionization (ES), matrix-assisted laser desorption ionization (MALDI) and fast atom bombardment (FAB). Upon ionization, several peaks are observed from one sample due to the formation of ions with different charges. Averaging the multiple readings of molecular mass obtained from a single mass spectrum affords an estimate of molecular mass of the bioagent identifying amplicon. Electrospray ionization mass spectrometry (ESI-MS) is particularly useful for very high molecular weight polymers such as proteins and nucleic acids having molecular weights greater than 10 kDa, since it yields a distribution of multiply-charged molecules of the sample without causing a significant amount of fragmentation.

The mass detectors used in the methods provided herein include, but are not limited to, Fourier transform ion cyclotron resonance mass spectrometry (FT-ICR-MS), time of flight (TOF), ion trap, quadrupole, magnetic sector, Q-TOF, and triple quadrupole.

Although the molecular mass of amplification products obtained using intelligent primers provides a means for identification of bioagents, conversion of molecular mass data to a base composition signature is useful for certain analyses. The base composition an the exact number of each nucleobase (A, T, C and G) in an oligonucleotide, for example, an amplicon, and can be calculated, for amplicons generated using the primer pairs provided here, from the molecular mass of the amplicons. In some embodiments, a base composition provides an index of a specific organism. Base compositions can be calculated from known sequences of known bioagent identifying amplicons and can also be experimentally determined by measuring the molecular mass of a given bioagent identifying amplicon, followed by determination of all possible base compositions which are consistent with the measured molecular mass within acceptable experimental error. The following example illustrates determination of base composition from an experimentally obtained molecular mass of a 46-mer amplification product originating at position 1337 of the 16S rRNA of Bacillus anthracis. The forward and reverse strands of the amplification product have measured molecular masses of 14208 and 14079 Da, respectively. The possible base compositions derived from the molecular masses of the forward and reverse strands for the B. anthracis products are listed in Table 1.

TABLE 1
Possible Base Compositions for B. anthracis 46mer Amplification Product

| | Mass | | | Mass | Base |
|---|---|---|---|---|---|
| Calc. Mass | Error | Base | Calc. Mass | Error | Composition |
| Forward | Forward | Composition of | Reverse | Reverse | of Reverse |
| Strand | Strand | Forward Strand | Strand | Strand | Strand |
| 14208.2935 | 0.079520 | A1 G17 C10 T18 | 14079.2624 | 0.080600 | A0 G14 C13 |
| | | | | | T19 |
| 14208.3160 | 0.056980 | A1 G20 C15 T10 | 14079.2849 | 0.058060 | A0 G17 C18 |
| | | | | | T11 |
| 14208.3386 | 0.034440 | A1 G23 C20 T2 | 14079.3075 | 0.035520 | A0 G20 C23 T3 |
| 14208.3074 | 0.065560 | A6 G11 C3 T26 | 14079.2538 | 0.089180 | A5 G5 C1 T35 |
| 14208.3300 | 0.043020 | A6 G14 C8 T18 | 14079.2764 | 0.066640 | A5 G8 C6 T27 |
| 14208.3525 | 0.020480 | A6 G17 C13 T10 | 14079.2989 | 0.044100 | A5 G11 C11 |
| | | | | | T19 |
| 14208.3751 | 0.002060 | A6 G20 C18 T2 | 14079.3214 | 0.021560 | A5 G14 C16 |
| | | | | | T11 |
| 14208.3439 | 0.029060 | A11 G8 C1 T26 | 14079.3440 | 0.000980 | A5 G17 C21 T3 |
| 14208.3665 | 0.006520 | A11 G11 C6 T18 | 14079.3129 | 0.030140 | A10 G5 C4 T27 |
| 14208.3890 | 0.016020 | A11 G14 C11 T10 | 14079.3354 | 0.007600 | A10 G8 C9 T19 |
| 14208.4116 | 0.038560 | A11 G17 C16 T2 | 14079.3579 | 0.014940 | A10 G11 C14 |
| | | | | | T11 |
| 14208.4030 | 0.029980 | A16 G8 C4 T18 | 14079.3805 | 0.037480 | A10 G14 C19 |
| | | | | | T3 |
| 14208.4255 | 0.052520 | A16 G11 C9 T10 | 14079.3494 | 0.006360 | A15 G2 C2 T27 |
| 14208.4481 | 0.075060 | A16 G14 C14 T2 | 14079.3719 | 0.028900 | A15 G5 C7 T19 |
| 14208.4395 | 0.066480 | A21 G5 C2 T18 | 14079.3944 | 0.051440 | A15 G8 C12 |
| | | | | | T11 |
| 14208.4620 | 0.089020 | A21 G8 C7 T10 | 14079.4170 | 0.073980 | A15 G11 C17 |
| | | | | | T3 |
| — | — | — | 14079.4084 | 0.065400 | A20 G2 C5 T19 |
| — | — | — | 14079.4309 | 0.087940 | A20 G5 C10 |
| | | | | | T13 |

Among the 16 possible base compositions for the forward strand and the 18 possible base compositions for the reverse strand that were calculated, only one pair (shown in bold) are complementary base compositions, which indicates the true base composition of the amplification product. It should be recognized that this logic is applicable for determination of base compositions of any bioagent identifying amplicon, regardless of the class of bioagent from which the corresponding amplification product was obtained.

In some embodiments, assignment of previously unobserved base compositions (also known as “true unknown base compositions”) to a given phylogeny can be accomplished via the use of pattern classifier model algorithms. Base compositions, like sequences, vary slightly from strain to strain within species, for example. In some embodiments, the pattern classifier model is the mutational probability model. On other embodiments, the pattern classifier is the polytope model. The mutational probability model and polytope model are both commonly owned and described in U.S. patent application Ser. No. 11/073,362 which is incorporated herein by reference in entirety.

In one embodiment, it is possible to manage this diversity by building “base composition probability clouds” around the composition constraints for each species. This permits identification of organisms in a fashion similar to sequence analysis. A “pseudo four-dimensional plot” can be used to visualize the concept of base composition probability clouds. Optimal primer design requires optimal choice of bioagent identifying amplicons and maximizes the separation between the base composition signatures of individual bioagents. Areas where clouds overlap indicate regions that may result in a misclassification, a problem which is overcome by a triangulation identification process using bioagent identifying amplicons not affected by overlap of base composition probability clouds.

In some embodiments, base composition probability clouds provide the means for screening potential primer pairs in order to avoid potential misclassifications of base compositions. In other embodiments, base composition probability clouds provide the means for predicting the identity of a bioagent whose assigned base composition was not previously observed and/or indexed in a bioagent identifying amplicon base composition database due to evolutionary transitions in its nucleic acid sequence. Thus, in contrast to probe-based techniques, mass spectrometry determination of base composition does not require prior knowledge of the composition or sequence in order to make the measurement.

Provided herein is bioagent classifying information similar to DNA sequencing and phylogenetic analysis at a level sufficient to identify a given bioagent and methods for obtaining such information. Furthermore, the process of determination of a previously unknown base composition for a given bioagent (for example, in a case where sequence information is unavailable) has downstream utility by providing additional bioagent indexing information with which to populate base composition databases. The process of future bioagent identification is thus greatly improved as more BCS indexes become available in base composition databases.

In some cases, a molecular mass of a single bioagent identifying amplicon alone does not provide enough resolution to unambiguously identify a given bioagent. The employment of more than one bioagent identifying amplicon for identification of a bioagent is herein referred to as “triangulation identification.” Triangulation identification is pursued by determining the molecular masses of a plurality of bioagent identifying amplicons selected within a plurality of housekeeping genes. This process is used to reduce false negative and false positive signals, and enable reconstruction of the origin of hybrid or otherwise engineered bioagents. For example, identification of the three part toxin genes typical of B. anthracis (Bowen et al., J. Appl. Microbiol., 1999, 87, 270-278) in the absence of the expected signatures from the B. anthracis genome would suggest a genetic engineering event.

In some embodiments, the triangulation identification process can be pursued by characterization of bioagent identifying amplicons in a massively parallel fashion using the polymerase chain reaction (PCR), such as multiplex PCR where multiple primers are employed in the same amplification reaction mixture, or PCR in multi-well plate format wherein a different and unique pair of primers is used in multiple wells containing otherwise identical reaction mixtures. Such multiplex and multi-well PCR methods are well known to those with ordinary skill in the arts of rapid throughput amplification of nucleic acids. In other related embodiments, one PCR reaction per well or container may be carried out, followed by an amplicon pooling step wherein the amplification products of different wells are combined in a single well or container which is then subjected to molecular mass analysis. The combination of pooled amplicons can be chosen such that the expected ranges of molecular masses of individual amplicons are not overlapping and thus will not complicate identification of signals.

In some embodiments, one or more nucleotide substitutions within a codon of a gene of an infectious organism confer drug resistance upon an organism which can be determined by codon base composition analysis. The organism can be a bacterium, virus, fungus or protozoan.

In some embodiments, the amplification product containing the codon being analyzed is of a length of about 35 to about 200 nucleobases. The primers employed in obtaining the amplification product can hybridize to upstream and downstream sequences directly adjacent to the codon, or can hybridize to upstream and downstream sequences one or more sequence positions away from the codon. The primers may have between about 70% to 100% sequence complementarity with the sequence of the gene containing the codon being analyzed.

In some embodiments, the codon base composition analysis is undertaken

In some embodiments, the codon analysis is undertaken for the purpose of investigating genetic disease in an individual. In other embodiments, the codon analysis is undertaken for the purpose of investigating a drug resistance mutation or any other deleterious mutation in an infectious organism such as a bacterium, virus, fungus or protozoan. In some embodiments, the bioagent is a bacterium identified in a biological product.

In some embodiments, the molecular mass of an amplification product containing the codon being analyzed is measured by mass spectrometry. The mass spectrometry can be either electrospray (ESI) mass spectrometry or matrix-assisted laser desorption ionization (MALDI) mass spectrometry. Time-of-flight (TOF) is an example of one mode of mass spectrometry compatible with the methods provided herein.

The methods provided here can also be employed to determine the relative abundance of drug resistant strains of the organism being analyzed. Relative abundances can be calculated from amplitudes of mass spectral signals with relation to internal calibrants. In some embodiments, known quantities of internal amplification calibrants can be included in the amplification reactions and abundances of analyte amplification product estimated in relation to the known quantities of the calibrants.

In some embodiments, upon identification of one or more drug-resistant strains of an infectious organism infecting an individual, one or more alternative treatments can be devised to treat the individual.

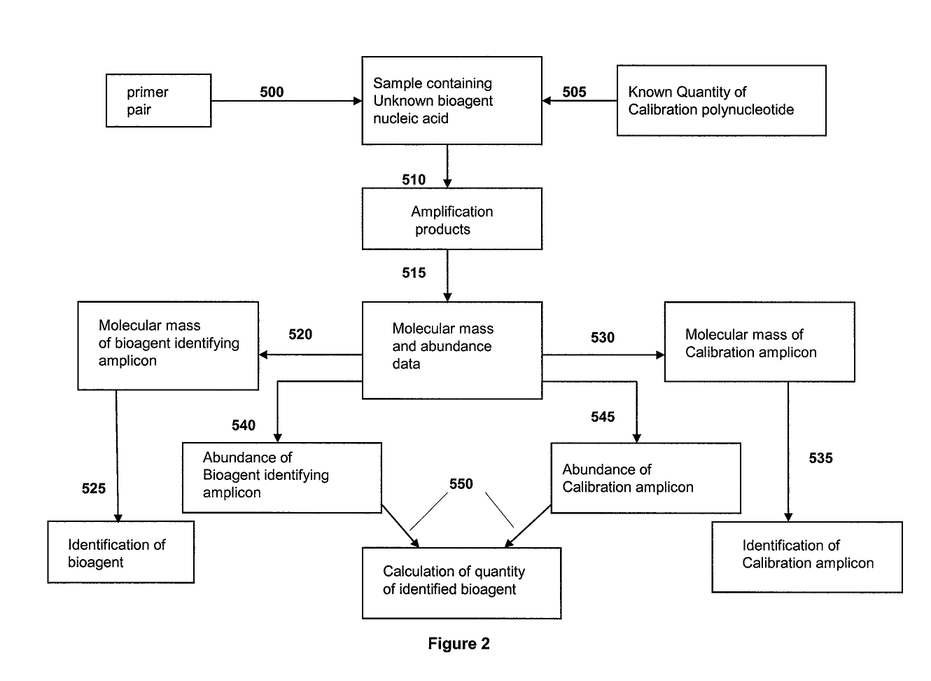

In some embodiments, the identity and quantity of an unknown bioagent can be determined using the process illustrated in FIG. 2. Primers (500) and a known quantity of a calibration polynucleotide (505) are added to a sample containing nucleic acid of an unknown bioagent. The total nucleic acid in the sample is then subjected to an amplification reaction (510) to obtain amplification products. The molecular masses of amplification products are determined (515) from which are obtained molecular mass and abundance data. The molecular mass of the bioagent identifying amplicon (520) provides the means for its identification (525) and the molecular mass of the calibration amplicon obtained from the calibration polynucleotide (530) provides the means for its identification (535). The abundance data of the bioagent identifying amplicon is recorded (540) and the abundance data for the calibration data is recorded (545), both of which are used in a calculation (550) which determines the quantity of unknown bioagent in the sample.

A sample comprising an unknown bioagent is contacted with a pair of primers that provide the means for amplification of nucleic acid from the bioagent, and a known quantity of a polynucleotide that comprises a calibration sequence. The nucleic acids of the bioagent and of the calibration sequence are amplified and the rate of amplification is reasonably assumed to be similar for the nucleic acid of the bioagent and of the calibration sequence. The amplification reaction then produces two amplification products: a bioagent identifying amplicon and a calibration amplicon. The bioagent identifying amplicon and the calibration amplicon should be distinguishable by molecular mass while being amplified at essentially the same rate. Effecting differential molecular masses can be accomplished by choosing as a calibration sequence, a representative bioagent identifying amplicon (from a specific species of bioagent) and performing, for example, a 2-8 nucleobase deletion or insertion within the variable region between the two priming sites. The amplified sample containing the bioagent identifying amplicon and the calibration amplicon is then subjected to molecular mass analysis by mass spectrometry, for example. The resulting molecular mass analysis of the nucleic acid of the bioagent and of the calibration sequence provides molecular mass data and abundance data for the nucleic acid of the bioagent and of the calibration sequence. The molecular mass data obtained for the nucleic acid of the bioagent enables identification of the unknown bioagent and the abundance data enables calculation of the quantity of the bioagent, based on the knowledge of the quantity of calibration polynucleotide contacted with the sample.

In some embodiments, construction of a standard curve where the amount of calibration polynucleotide spiked into the sample is varied provides additional resolution and improved confidence for the determination of the quantity of bioagent in the sample. The use of standard curves for analytical determination of molecular quantities is well known to one with ordinary skill and can be performed without undue experimentation.

In some embodiments, multiplex amplification is performed where multiple bioagent identifying amplicons are amplified with multiple primer pairs which also amplify the corresponding standard calibration sequences. In this or other embodiments, the standard calibration sequences are optionally included within a single vector which functions as the calibration polynucleotide. Multiplex amplification methods are well known to those with ordinary skill and can be performed without undue experimentation.

In some embodiments, the calibrant polynucleotide is used as an internal positive control to confirm that amplification conditions and subsequent analysis steps are successful in producing a measurable amplicon. Even in the absence of copies of the genome of a bioagent, the calibration polynucleotide should give rise to a calibration amplicon. Failure to produce a measurable calibration amplicon indicates a failure of amplification or subsequent analysis step such as amplicon purification or molecular mass determination. Reaching a conclusion that such failures have occurred is in itself, a useful event.

In some embodiments, the calibration sequence is comprised of DNA. In some embodiments, the calibration sequence is comprised of RNA.

In some embodiments, the calibration sequence is inserted into a vector that itself functions as the calibration polynucleotide. In some embodiments, more than one calibration sequence is inserted into the vector that functions as the calibration polynucleotide. Such a calibration polynucleotide is herein termed a “combination calibration polynucleotide.” The process of inserting polynucleotides into vectors is routine to those skilled in the art and can be accomplished without undue experimentation. Thus, it should be recognized that the calibration method should not be limited to the embodiments described herein. The calibration method can be applied for determination of the quantity of any bioagent identifying amplicon when an appropriate standard calibrant polynucleotide sequence is configured and used. The process of choosing an appropriate vector for insertion of a calibrant is also a routine operation that can be accomplished by one with ordinary skill without undue experimentation.

In some embodiments, the primer pairs produce bioagent identifying amplicons within stable and highly conserved regions of bacteria. The advantage to characterization of an amplicon defined by priming regions that fall within a highly conserved region is that there is a low probability that the region will evolve past the point of primer recognition, in which case, the primer hybridization of the amplification step would fail. Such a primer set is thus useful as a broad range survey-type primer. In another embodiment, the primers produce bioagent identifying amplicons including a region which evolves more quickly than the stable region described above. The advantage of characterization bioagent identifying amplicon corresponding to an evolving genomic region is that it is useful for distinguishing emerging strain variants or the presence of virulence genes, drug resistance genes, or codon mutations that induce drug resistance.

The embodiments provided here also have significant advantages in providing a platform for identification of diseases caused by emerging bacterial strains such as, for example, drug-resistant strains of Staphylococcus aureus. The present embodiments eliminate the need for prior knowledge of bioagent sequence to generate hybridization probes. This is possible because the methods are not confounded by naturally occurring evolutionary variations occurring in the sequence acting as the template for production of the bioagent identifying amplicon. Measurement of molecular mass and determination of base composition is accomplished in an unbiased manner without sequence prejudice.

Another embodiment provides a means of tracking the spread of a bacterium, such as a particular drug-resistant strain when a plurality of samples obtained from different locations are analyzed by the methods described above in an epidemiological setting. In one embodiment, a plurality of samples from a plurality of different locations is analyzed with primer pairs which produce bioagent identifying amplicons, a subset of which contains a specific drug-resistant bacterial strain. The corresponding locations of the members of the drug-resistant strain subset indicate the spread of the specific drug-resistant strain to the corresponding locations.

Also provided herein are kits for carrying out the methods described herein. In some embodiments, the kit may comprise a sufficient quantity of one or more primer pairs to perform an amplification reaction on a target polynucleotide from a bioagent to form a bioagent identifying amplicon. In some embodiments, the kit may comprise from one to fifty primer pairs, from one to twenty primer pairs, from one to ten primer pairs, or from two to five primer pairs. In some embodiments, the kit may comprise one or more primer pairs recited in Table 2.

In some embodiments, the kit comprises one or more broad range survey primer(s), division wide primer(s), or drill-down primer(s), or any combination thereof. If a given problem involves identification of a specific bioagent, the solution to the problem may require the selection of a particular combination of primers to provide the solution to the problem. A kit may be configured so as to comprise particular primer pairs for identification of a particular bioagent. A drill-down kit may be used, for example, to distinguish different genotypes or strains, drug-resistant, or otherwise. In some embodiments, the primer pair components of any of these kits may be additionally combined to comprise additional combinations of broad range survey primers and division-wide primers so as to be able to identify a bacterium.

In some embodiments, the kit contains standardized calibration polynucleotides for use as internal amplification calibrants. Internal calibrants are described in commonly owned PCT pre-grant publication, publication number WO 2005/094421, which is incorporated herein by reference in its entirety.

In some embodiments, the kit comprises a sufficient quantity of reverse transcriptase (if RNA is to be analyzed for example), a DNA polymerase, suitable nucleoside triphosphates (including alternative dNTPs such as inosine or modified dNTPs such as the 5-propynyl pyrimidines or any dNTP containing molecular mass-modifying tags such as those described above), a DNA ligase, and/or reaction buffer, or any combination thereof, for the amplification processes described above. A kit may further include instructions pertinent for the particular embodiment of the kit, such instructions describing the primer pairs and amplification conditions for operation of the method. A kit may also comprise amplification reaction containers such as microcentrifuge tubes and the like. A kit may also comprise reagents or other materials for isolating bioagent nucleic acid or bioagent identifying amplicons from amplification, including, for example, detergents, solvents, or ion exchange resins which may be linked to magnetic beads. A kit may also comprise a table of measured or calculated molecular masses and/or base compositions of bioagents using the primer pairs of the kit.

In some embodiments, a kit may contain one or more survey bacterial primer pairs and one or more triangulation genotyping analysis primer pairs such as the primer pairs of Tables 8, 12, 14, 19, 21, 23, or 24. In some embodiments, the kit may represent a less expansive genotyping analysis but include triangulation genotyping analysis primer pairs for more than one genus or species of bacteria. For example, a kit for surveying nosocomial infections at a health care facility may include, for example, one or more broad range survey primer pairs, one or more division wide primer pairs, one or more Acinetobacter baumannii triangulation genotyping analysis primer pairs and one or more Staphylococcus aureus triangulation genotyping analysis primer pairs. One with ordinary skill will be capable of analyzing in silico amplification data to determine which primer pairs will be able to provide optimal identification resolution for the bacterial bioagents of interest.

In some embodiments, a kit may be assembled for identification of strains of bacteria involved in contamination of food. An example of such a kit embodiment is a kit comprising one or more bacterial survey primer pairs of Table 5 with one or more triangulation genotyping analysis primer pairs of Table 12 which provide strain resolving capabilities for identification of specific strains of Campylobacter jejuni.

Some embodiments of the kits are 96-well or 384-well plates with a plurality of wells containing any or all of the following components: dNTPs, buffer salts, Mg²⁺, betaine, and primer pairs. In some embodiments, a polymerase is also included in the plurality of wells of the 96-well or 384-well plates.

Some embodiments of the kit contain instructions for PCR and mass spectrometry analysis of amplification products obtained using the primer pairs of the kits.

Some embodiments of the kit include a barcode which uniquely identifies the kit and the components contained therein according to production lots and may also include any other information relative to the components such as concentrations, storage temperatures, etc. The barcode may also include analysis information to be read by optical barcode readers and sent to a computer controlling amplification, purification and mass spectrometric measurements. In some embodiments, the barcode provides access to a subset of base compositions in a base composition database which is in digital communication with base composition analysis software such that a base composition measured with primer pairs from a given kit can be compared with known base compositions of bioagent identifying amplicons defined by the primer pairs of that kit.

In some embodiments, the kit contains a database of base compositions of bioagent identifying amplicons defined by the primer pairs of the kit. The database is stored on a convenient computer readable medium such as a compact disk or USB drive, for example.

In some embodiments, the kit includes a computer program stored on a computer formatted medium (such as a compact disk or portable USB disk drive, for example) comprising instructions which direct a processor to analyze data obtained from the use of the primer pairs provided herein. The instructions of the software transform data related to amplification products into a molecular mass or base composition which is a useful concrete and tangible result used in identification and/or classification of bioagents. In some embodiments, the kits of the present invention contain all of the reagents sufficient to carry out one or more of the methods described herein.

The following examples serve only to illustrate the embodiments provided herein and are not intended to be limiting. In order that the embodiments disclosed herein may be more efficiently understood, examples are provided below. It should be understood that these examples are for illustrative purposes only and are not to be construed as limiting in any manner.

### EXAMPLES

### Example 1

### Design and Validation of Primers that Define Bioagent Identifying Amplicons for Identification of Bacteria

For design of primers that define bacterial bioagent identifying amplicons, a series of bacterial genome segment sequences were obtained, aligned and scanned for regions where pairs of PCR primers would amplify products of about 45 to about 150 nucleotides in length and distinguish subgroups and/or individual strains from each other by their molecular masses or base compositions. A typical process shown in FIG. 1 is employed for this type of analysis.

A database of expected base compositions for each primer region was generated using an in silico PCR search algorithm, such as (ePCR). An existing RNA structure search algorithm (Macke et al., Nucl. Acids Res., 2001, 29, 4724-4735, which is incorporated herein by reference in its entirety) has been modified to include PCR parameters such as hybridization conditions, mismatches, and thermodynamic calculations (SantaLucia, Proc. Natl. Acad. Sci. U.S.A., 1998, 95, 1460-1465, which is incorporated herein by reference in its entirety). This also provides information on primer specificity of the selected primer pairs.

Table 2 represents a collection of primers (sorted by primer pair number) configured to identify bacteria using the methods described herein. The primer pair number is an in-house database index number. Conserved regions which primers were configured to hybridize within were identified on bacterial bioagent genes including, for example, arcC, aroE, ermA, ermC, gmk, gyrA, mecA, mecR1, mupR, nuc, pta, pvluk, tpi, tsst, tufB, and yqi. The forward and reverse primer names shown in Table 1 indicate the gene region of a bacterial genome to which the forward and reverse primers hybridize relative to a reference sequence. The forward primer name TSST1_NC002758.2-2137509-2138213_{—}519_{—}546_F indicates that the forward primer (“_F”) hybridizes to the GyrA gene (“GYRA”), specifically to residues 519-546 (“519_{—}546”) of a reference sequence represented by a sequence extraction of coordinates 2137509-2138213 from GenBank gi number 57634611 (as indicated by cross-references in Table 2 for the prefix “GYRA_NC002953”). This sequence extraction reference includes sequence encoding for tsst. The primer pair name codes appearing in Table 2 are defined in Table 3. For example, Table 2 lists gene abbreviations and GenBank gi numbers that correspond with each primer name code. For example, for the above-mentioned primer pair has the code “TSST1_NC002758.2” and is thus configured to hybridize to sequence encoding the tsst gene, and the extraction sequence corresponds to coordinates 2137509-2138213 from GenBank gi number 57634611, which is a Staphylococcus aureus sequence. One of skill in the art will understand how to determine the exact hybridization coordinates of the primers with respect to the GenBank sequences, given this information. The reference nomenclature in the primer name is selected to provide a reference, and does not necessarily mean that the primer pair has been configured with 100% complementarity to that target site on the reference sequence. One with ordinary skill knows how to obtain individual gene sequences or portions thereof from genomic sequences present in GenBank. In Table 2, Tp=5-propynyluracil; Cp=5-propynylcytosine; *=phosphorothioate linkage; I=inosine. T GenBank Accession Numbers for reference sequences of bacteria are shown in Table 3 (below). In some cases, the reference sequences are extractions from bacterial genomic sequences or complements thereof. A description of the primer design is provided herein. In some cases, the reference sequences are extractions from bacterial genomic sequences or complements thereof.

TABLE 2
Primer Pairs for Identification of Bacteria

| Primer | | | Forward | | | Reverse |
|---|---|---|---|---|---|---|
| Pair | Forward Primer | | SEQ ID | | | SEQ ID |
| Number | Name | Forward Sequence | NO: | Reverse Primer Name | Reverse Sequence | NO: |
| 1 | 16S_EC_1077_1106_F | GTGAGATGTTGGGTTAAGT | 134 | 16S_EC_1175_1195_R | GACGTCATCCCCACCTTCCTC | 809 |
| | | CCCGTAACGAG | | | | |
| 2 | 16S_EC_1082_1106_F | ATGTTGGGTTAAGTCCCGC | 38 | 16S_EC_1175_1197_R | TTGACGTCATCCCCACCTTCCTC | 1398 |
| | | AACGAG | | | | |
| 3 | 16S_EC_1090_1111_F | TTAAGTCCCGCAACGATCG | 651 | 16S_EC_1175_1196_R | TGACGTCATCCCCACCTTCCTC | 1159 |
| | | CAA | | | | |
| 4 | 16S_EC_1222_1241_F | GCTACACACGTGCTACAATG | 114 | 16S_EC_1303_1323_R | CGAGTTGCAGACTGCGATCCG | 787 |
| 5 | 16S_EC_1332_1353_F | AAGTCGGAATCGCTAGTAA | 10 | 16S_EC_1389_1407_R | GACGGGCGGTGTGTACAAG | 806 |
| | | TCG | | | | |
| 6 | 16S_EC_30_54_F | TGAACGCTGGTGGCATGCT | 429 | 16S_EC_105_126_R | TACGCATTACTCACCCGTCCGC | 897 |
| | | TAACAC | | | | |
| 7 | 16S_EC_38_64_F | GTGGCATGCCTAATACATG | 136 | 16S_EC_101_120_R | TTACTCACCCGTCCGCCGCT | 1365 |
| | | CAAGTCG | | | | |
| 8 | 16S_EC_49_68_F | TAACACATGCAAGTCGAACG | 152 | 16S_EC_104_120_R | TTACTCACCCGTCCGCC | 1364 |
| 9 | 16S_EC_683_700_F | GTGTAGCGGTGAAATGCG | 137 | 16S_EC_774_795_R | GTATCTAATCCTGTTTGCTCCC | 839 |
| 10 | 16S_EC_713_732_F | AGAACACCGATGGCGAAGGC | 21 | 16S_EC_789_809_R | CGTGGACTACCAGGGTATCTA | 798 |
| 11 | 16S_EC_785_806_F | GGATTAGAGACCCTGGTAG | 118 | 16S_EC_880_897_R | GGCCGTACTCCCCAGGCG | 830 |
| | | TCC | | | | |
| 12 | 16S_EC_785_810_F | GGATTAGATACCCTGGTAG | 119 | 16S_EC_880_897_2_R | GGCCGTACTCCCCAGGCG | 830 |
| | | TCCACGC | | | | |
| 13 | 16S_EC_789_810_F | TAGATACCCTGGTAGTCCA | 206 | 16S_EC_880_894_R | CGTACTCCCCAGGCG | 796 |
| | | CGC | | | | |
| 14 | 16S_EC_960_981_F | TTCGATGCAACGCGAAGAA | 672 | 16S_EC_1054_1073_R | ACGAGCTGACGACAGCCATG | 735 |
| | | CCT | | | | |
| 15 | 16S_EC_969_985_F | ACGCGAAGAACCTTACC | 19 | 16S_EC_1061_1078_R | ACGACACGAGCTGACGAC | 734 |
| 16 | 23S_EC_1826_1843_F | CTGACACCTGCCCGGTGC | 80 | 23S_EC_1906_1924_R | GACCGTTATAGTTACGGCC | 805 |
| 17 | 23S_EC_2645_2669_F | TCTGTCCCTAGTACGAGAG | 408 | 23S_EC_2744_2761_R | TGCTTAGATGCTTTCAGC | 1252 |
| | | GACCGG | | | | |
| 18 | 23S_EC_2645_2669_2_F | CTGTCCCTAGTACGAGAGG | 83 | 23S_EC_2751_2767_R | GTTTCATGCTTAGATGCTTTCAGC | 846 |
| | | ACCGG | | | | |
| 19 | 23S_EC_493_518_F | GGGGAGTGAAAGAGATCCT | 125 | 23S_EC_551_571_R | ACAAAAGGTACGCCGTCACCC | 717 |
| | | GAAACCG | | | | |
| 20 | 23S_EC_493_518_2_F | GGGGAGTGAAAGAGATCCT | 125 | 23S_EC_551_571_2_R | ACAAAAGGCACGCCATCACCC | 716 |
| | | GAAACCG | | | | |
| 21 | 23S_EC_971_992_F | CGAGAGGGAAACAACCCAG | 66 | 23S_EC_1059_1077_R | TGGCTGCTTCTAAGCCAAC | 1282 |
| | | ACC | | | | |
| 22 | CAPC_BA_104_131_F | GTTATTTAGCACTCGTTTT | 139 | CAPC_BA_180_205_R | TGAATCTTGAAACACCATACGTA | 1150 |
| | | TAATCAGCC | | | ACG | |
| 23 | CAPC_BA_114_133_F | ACTCGTTTTTAATCAGCCCG | 20 | CAPC_BA_185_205_R | TGAATCTTGAAACACCATACG | 1149 |
| 24 | CAPC_BA_274_303_F | GATTATTGTTATCCTGTTA | 109 | CAPC_BA_349_376_R | GTAACCCTTGTCTTTGAATTGTA | 837 |
| | | TGCCATTTGAG | | | TTTGC | |
| 25 | CAPC_BA_276_296_F | TTATTGTTATCCTGTTATG | 663 | CAPC_BA_358_377_R | GGTAACCCTTGTCTTTGAAT | 834 |
| | | CC | | | | |
| 26 | CAPC_BA_281_301_F | GTTATCCTGTTATGCCATT | 138 | CAPC_BA_361_378_R | TGGTAACCCTTGTCTTTG | 1298 |
| | | TG | | | | |
| 27 | CAPC_BA_315_334_F | CCGTGGTATTGGAGTTATTG | 59 | CAPC_BA_361_378_R | TGGTAACCCTTGTCTTTG | 1298 |
| 28 | CYA_BA_1055_1072_F | GAAAGAGTTCGGATTGGG | 92 | CYA_BA_1112_1130_R | TGTTGACCATGCTTCTTAG | 1352 |
| 29 | CYA_BA_1349_1370_F | ACAACGAAGTACAATACAA | 12 | CYA_BA_1447_1426_R | CTTCTACATTTTTAGCCATCAC | 800 |
| | | GAC | | | | |
| 30 | CYA_BA_1353_1379_F | CGAAGTACAATACAAGACA | 64 | CYA_BA_1448_1467_R | TGTTAACGGCTTCAAGACCC | 1342 |
| | | AAAGAAGG | | | | |
| 31 | CYA_BA_1359_1379_F | ACAATACAAGACAAAAGAA | 13 | CYA_BA_1447_1461_R | CGGCTTCAAGACCCC | 794 |
| | | GG | | | | |
| 32 | CYA_BA_914_937_F | CAGGTTTAGTACCAGAACA | 53 | CYA_BA_999_1026_R | ACCACTTTTAATAAGGTTTGTAG | 728 |
| | | TGCAG | | | CTAAC | |
| 33 | CYA_BA_916_935_F | GGTTTAGTACCAGAACATGC | 131 | CYA_BA_1003_1025_R | CCACTTTTAATAAGGTTTGTAGC | 768 |
| 34 | INFB_EC_1365_1393_F | TGCTCGTGGTGCACAAGTA | 524 | INFB_EC_1439_1467_R | TGCTGCTTTCGCATGGTTAATTG | 1248 |
| | | ACGGATATTA | | | CTTCAA | |
| 35 | LEF_BA_1033_1052_F | TCAAGAAGAAAAAGAGC | 254 | LEF_BA_1119_1135_R | GAATATCAATTTGTAGC | 803 |
| 36 | LEF_BA_1036_1066_F | CAAGAAGAAAAAGAGCTTC | 44 | LEF_BA_1119_1149_R | AGATAAAGAATCACGAATATCAA | 745 |
| | | TAAAAAGAATAC | | | TTTGTAGC | |
| 37 | LEF_BA_756_781_F | AGCTTTTGCATATTATATC | 26 | LEF_BA_843_872_R | TCTTCCAAGGATAGATTTATTTC | 1135 |
| | | GAGCCAC | | | TTGTTCG | |
| 38 | LEF_BA_758_778_F | CTTTTGCATATTATATCGA | 90 | LEF_BA_843_865_R | AGGATAGATTTATTTCTTGTTCG | 748 |
| | | GC | | | | |
| 39 | LEF_BA_795_813_F | TTTACAGCTTTATGCACCG | 700 | LEF_BA_883_900_R | TCTTGACAGCATCCGTTG | 1140 |
| 40 | LEF_BA_883_899_F | CAACGGATGCTGGCAAG | 43 | LEF_BA_939_958_R | CAGATAAAGAATCGCTCCAG | 762 |
| 41 | PAG_BA_122_142_F | CAGAATCAAGTTCCCAGGGG | 49 | PAG_BA_190_209_R | CCTGTAGTAGAAGAGGTAAC | 781 |
| 42 | PAG_BA_123_145_F | AGAATCAAGTTCCCAGGGG | 22 | PAG_BA_187_210_R | CCCTGTAGTAGAAGAGGTAACCAC | 774 |
| | | TTAC | | | | |
| 43 | PAG_BA_269_287_F | AATCTGCTATTTGGTCAGG | 11 | PAG_BA_326_344_R | TGATTATCAGCGGAAGTAG | 1186 |
| 44 | PAG_BA_655_675_F | GAAGGATATACGGTTGATG | 93 | PAG_BA_755_772_R | CCGTGCTCCATTTTTCAG | 778 |
| | | TC | | | | |
| 45 | PAG_BA_753_772_F | TCCTGAAAAATGGAGCACGG | 341 | PAG_BA_849_868_R | TCGGATAAGCTGCCACAAGG | 1089 |
| 46 | PAG_BA_763_781_F | TGGAGCACGGCTTCTGATC | 552 | PAG_BA_849_868_R | TCGGATAAGCTGCCACAAGG | 1089 |
| 47 | RPOC_EC_1018_1045_F | CAAAACTTATTAGGTAAGC | 39 | RPOC_EC_1095_1124_R | TCAAGCGCCATTTCTTTTGGTAA | 959 |
| | | GTGTTGACT | | | ACCACAT | |
| 48 | RPOC_EC_1018_1045_2_F | CAAAACTTATTAGGTAAGC | 39 | RPOC_EC_1095_1124_2_R | TCAAGCGCCATCTCTTTCGGTAA | 958 |
| | | GTGTTGACT | | | TCCACAT | |
| 49 | RPOC_EC_114_140_F | TAAGAAGCCGGAAACCATC | 158 | RPOC_EC_213_232_R | GGCGCTTGTACTTACCGCAC | 831 |
| | | AACTACCG | | | | |
| 50 | RPOC_EC_2178_2196_F | TGATTCTGGTGCCCGTGGT | 478 | RPOC_EC_2225_2246_R | TTGGCCATCAGGCCACGCATAC | 1414 |
| 51 | RPOC_EC_2178_2196_2_F | TGATTCCGGTGCCCGTGGT | 477 | RPOC_EC_2225_2246_2_R | TTGGCCATCAGACCACGCATAC | 1413 |
| 52 | RPOC_EC_2218_2241_F | CTGGCAGGTATGCGTGGTC | 81 | RPOC_EC_2313_2337_R | CGCACCGTGGGTTGAGATGAAGT | 790 |
| | | TGATG | | | AC | |
| 53 | RPOC_EC_2218_2241_2_F | CTTGCTGGTATGCGTGGTC | 86 | RPOC_EC_2313_2337_2_R | CGCACCATGCGTAGAGATGAAGT | 789 |
| | | TGATG | | | AC | |
| 54 | RPOC_EC_808_833_F | CGTCGGGTGATTAACCGTA | 75 | RPOC_EC_865_889_R | GTTTTTCGTTGCGTACGATGATG | 847 |
| | | ACAACCG | | | TC | |
| 55 | RPOC_EC_808_833_2_F | CGTCGTGTAATTAACCGTA | 76 | RPOC_EC_865_891_R | ACGTTTTTCGTTTTGAACGATAA | 741 |
| | | ACAACCG | | | TGCT | |
| 56 | RPOC_EC_993_1019_F | CAAAGGTAAGCAAGGTCGT | 41 | RPOC_EC_1036_1059_R | CGAACGGCCTGAGTAGTCAACACG | 785 |
| | | TTCCGTCA | | | | |
| 57 | RPOC_EC_993_1019_2_F | CAAAGGTAAGCAAGGACGT | 40 | RPOC_EC_1036_1059_2_R | CGAACGGCCAGAGTAGTCAACACG | 784 |
| | | TTCCGTCA | | | | |
| 58 | SSPE_BA_115_137_F | CAAGCAAACGCACAATCAG | 45 | SSPE_BA_197_222_R | TGCACGTCTGTTTCAGTTGCAAA | 1201 |
| | | AAGC | | | TTC | |
| 59 | TUFB_EC_239_259_F | TAGACTGCCCAGGACACGC | 204 | TUFB_EC_283_303_R | GCCGTCCATCTGAGCAGCACC | 815 |
| | | TG | | | | |
| 60 | TUFB_EC_239_259_2_F | TTGACTGCCCAGGTCACGC | 678 | TUFB_EC_283_303_2_R | GCCGTCCATTTGAGCAGCACC | 816 |
| | | TG | | | | |
| 61 | TUFB_EC_976_1000_F | AACTACCGTCCGCAGTTCT | 4 | TUFB_EC_1045_1068_R | GTTGTCGCCAGGCATAACCATTTC | 845 |
| | | ACTTCC | | | | |
| 62 | TUFB_EC_976_1000_2_F | AACTACCGTCCTCAGTTCT | 5 | TUFB_EC_1045_1068_2_R | GTTGTCACCAGGCATTACCATTTC | 844 |
| | | ACTTCC | | | | |
| 63 | TUFB_EC_985_1012_F | CCACAGTTCTACTTCCGTA | 56 | TUFB_EC_1033_1062_R | TCCAGGCATTACCATTTCTACTC | 1006 |
| | | CTACTGACG | | | CTTCTGG | |
| 66 | RPLB_EC_650_679_F | GACCTACAGTAAGAGGTTC | 98 | RPLB_EC_739_762_R | TCCAAGTGCTGGTTTACCCCATGG | 999 |
| | | TGTAATGAACC | | | | |
| 67 | RPLB_EC_688_710_F | CATCCACACGGTGGTGGTG | 54 | RPLB_EC_736_757_R | GTGCTGGTTTACCCCATGGAGT | 842 |
| | | AAGG | | | | |
| 68 | RPOC_EC_1036_1060_F | CGTGTTGACTATTCGGGGC | 78 | RPOC_EC_1097_1126_R | ATTCAAGAGCCATTTCTTTTGGT | 754 |
| | | GTTCAG | | | AAACCAC | |
| 69 | RPOB_EC_3762_3790_F | TCAACAACCTCTTGGAGGT | 248 | RPOB_EC_3836_3865_R | TTTCTTGAAGAGTATGAGCTGCT | 1435 |
| | | AAAGCTCAGT | | | CCGTAAG | |
| 70 | RPLB_EC_688_710_F | CATCCACACGGTGGTGGTG | 54 | RPLB_EC_743_771_R | TGTTTTGTATCCAAGTGCTGGTT | 1356 |
| | | AAGG | | | TACCCC | |
| 71 | VALS_EC_1105_1124_F | CGTGGCGGCGTGGTTATCGA | 77 | VALS_EC_1195_1218_R | CGGTACGAACTGGATGTCGCCGTT | 795 |
| 72 | RPOB_EC_1845_1866_F | TATCGCTCAGGCGAACTCC | 233 | RPOB_EC_1909_1929_R | GCTGGATTCGCCTTTGCTACG | 825 |
| | | AAC | | | | |
| 73 | RPLB_EC_669_698_F | TGTAATGAACCCTAATGAC | 623 | RPLB_EC_735_761_R | CCAAGTGCTGGTTTACCCCATGG | 767 |
| | | CATCCACACGG | | | AGTA | |
| 74 | RPLB_EC_671_700_F | TAATGAACCCTAATGACCA | 169 | RPLB_EC_737_762_R | TCCAAGTGCTGGTTTACCCCATG | 1000 |
| | | TCCACACGGTG | | | GAG | |
| 75 | SP101_SPET11_1_29_F | AACCTTAATTGGAAAGAAA | 2 | SP101_SPET11_92_116_R | CCTACCCAACGTTCACCAAGGGC | 779 |
| | | CCCAAGAAGT | | | AG | |
| 76 | SP101_SPET11_118_147_F | GCTGGTGAAAATAACCCAG | 115 | SP101_SPET11_213_238_R | TGTGGCCGATTTCACCACCTGCT | 1340 |
| | | ATGTCGTCTTC | | | CCT | |
| 77 | SP101_SPET11_216_243_F | AGCAGGTGGTGAAATCGGC | 24 | SP101_SPET11_308_333_R | TGCCACTTTGACAACTCCTGTTG | 1209 |
| | | CACATGATT | | | CTG | |
| 78 | SP101_SPET11_266_295_F | CTTGTACTTGTGGCTCACA | 89 | SP101_SPET11_355_380_R | GCTGCTTTGATGGCTGAATCCCC | 824 |
| | | CGGCTGTTTGG | | | TTC | |
| 79 | SP101_SPET11_322_344_F | GTCAAAGTGGCACGTTTAC | 132 | SP101_SPET11_423_441_R | ATCCCCTGCTTCTGCTGCC | 753 |
| | | TGGC | | | | |
| 80 | SP101_SPET11_358_387_F | GGGGATTCAGCCATCAAAG | 126 | SP101_SPET11_448_473_R | CCAACCTTTTCCACAACAGAATC | 766 |
| | | CAGCTATTGAC | | | AGC | |
| 81 | SP101_SPET11_600_629_F | CCTTACTTCGAACTATGAA | 62 | SP101_SPET11_686_714_R | CCCATTTTTTCACGCATGCTGAA | 772 |
| | | TCTTTTGGAAG | | | AATATC | |
| 82 | SP101_SPET11_658_684_F | GGGGATTGATATCACCGAT | 127 | SP101_SPET11_756_784_R | GATTGGCGATAAAGTGATATTTT | 813 |
| | | AAGAAGAA | | | CTAAAA | |
| 83 | SP101_SPET11_776_801_F | TCGCCAATCAAAACTAAGG | 364 | SP101_SPET11_871_896_R | GCCCACCAGAAAGACTAGCAGGA | 814 |
| | | GAATGGC | | | TAA | |
| 84 | SP101_SPET11_893_921_F | GGGCAACAGCAGCGGATTG | 123 | SP101_SPET11_988_1012_R | CATGACAGCCAAGACCTCACCCA | 763 |
| | | CGATTGCGCG | | | CC | |
| 85 | SP101_SPET11_1154_1179_F | CAATACCGCAACAGCGGTG | 47 | SP101_SPET11_1251_1277_R | GACCCCAACCTGGCCTTTTGTCG | 804 |
| | | GCTTGGG | | | TTGA | |
| 86 | SP101_SPET11_1314_1336_F | CGCAAAAAAATCCAGCTAT | 68 | SP101_SPET11_1403_1431_R | AAACTATTTTTTTAGCTATACTC | 711 |
| | | TAGC | | | GAACAC | |
| 87 | SP101_SPET11_1408_1437_F | CGAGTATAGCTAAAAAAAT | 67 | SP101_SPET11_1486_1515_R | GGATAATTGGTCGTAACAAGGGA | 828 |
| | | AGTTTATGACA | | | TAGTGAG | |
| 88 | SP101_SPET11_1688_1716_F | CCTATATTAATCGTTTACA | 60 | SP101_SPET11_1783_1808_R | ATATGATTATCATTGAACTGCGG | 752 |
| | | GAAACTGGCT | | | CCG | |
| 89 | SP101_SPET11_1711_1733_F | CTGGCTAAAACTTTGGCAA | 82 | SP101_SPET11_1808_1835_R | GCGTGACGACCTTCTTGAATTGT | 821 |
| | | CGGT | | | AATCA | |
| 90 | SP101_SPET11_1807_1835_F | ATGATTACAATTCAAGAAG | 33 | SP101_SPET11_1901_1927_R | TTGGACCTGTAATCAGCTGAATA | 1412 |
| | | GTCGTCACGC | | | CTGG | |
| 91 | SP101_SPET11_1967_1991_F | TAACGGTTATCATGGCCCA | 155 | SP101_SPET11_2062_2083_R | ATTGCCCAGAAATCAAATCATC | 755 |
| | | GATGGG | | | | |
| 92 | SP101_SPET11_2260_2283_F | CAGAGACCGTTTTATCCTA | 50 | SP101_SPET11_2375_2397_R | TCTGGGTGACCTGGTGTTTTAGA | 1131 |
| | | TCAGC | | | | |
| 93 | SP101_SPET11_2375_2399_F | TCTAAAACACCAGGTCACC | 390 | SP101_SPET11_2470_2497_R | AGCTGCTAGATGAGCTTCTGCCA | 747 |
| | | CAGAAG | | | TGGCC | |
| 94 | SP101_SPET11_2468_2487_F | ATGGCCATGGCAGAAGCTCA | 35 | SP101_SPET11_2543_2570_R | CCATAAGGTCACCGTCACCATTC | 770 |
| | | | | | AAAGC | |
| 95 | SP101_SPET11_2961_2984_F | ACCATGACAGAAGGCATTT | 15 | SP101_SPET11_3023_3045_R | GGAATTTACCAGCGATAGACACC | 827 |
| | | TGACA | | | | |
| 96 | SP101_SPET11_3075_3103_F | GATGACTTTTTAGCTAATG | 108 | SP101_SPET11_3168_3196_R | AATCGACGACCATCTTGGAAAGA | 715 |
| | | GTCAGGCAGC | | | TTTCTC | |
| 97 | SP101_SPET11_3386_3403_F | AGCGTAAAGGTGAACCTT | 25 | SP101_SPET11_3480_3506_R | CCAGCAGTTACTGTCCCCTCATC | 769 |
| | | | | | TTTG | |
| 98 | SP101_SPET11_3511_3535_F | GCTTCAGGAATCAATGATG | 116 | SP101_SPET11_3605_3629_R | GGGTCTACACCTGCACTTGCATA | 832 |
| | | GAGCAG | | | AC | |
| 111 | RPOB_EC_3775_3803_F | CTTGGAGGTAAGTCTCATT | 87 | RPOB_EC_3829_3858_R | CGTATAAGCTGCACCATAAGCTT | 797 |
| | | TTGGTGGGCA | | | GTAATGC | |
| 112 | VALS_EC_1833_1850_F | CGACGCGCTGCGCTTCAC | 65 | VALS_EC_1920_1943_R | GCGTTCCACAGCTTGTTGCAGAAG | 822 |
| 113 | RPOB_EC_1336_1353_F | GACCACCTCGGCAACCGT | 97 | RPOB_EC_1438_1455_R | TTCGCTCTCGGCCTGGCC | 1386 |
| 114 | TUFB_EC_225_251_F | GCACTATGCACACGTAGAT | 111 | TUFB_EC_284_309_R | TATAGCACCATCCATCTGAGCGG | 930 |
| | | TGTCCTGG | | | CAC | |
| 115 | DNAK_EC_428_449_F | CGGCGTACTTCAACGACAG | 72 | DNAK_EC_503_522_R | CGCGGTCGGCTCGTTGATGA | 792 |
| | | CCA | | | | |
| 116 | VALS_EC_1920_1943_F | CTTCTGCAACAAGCTGTGG | 85 | VALS_EC_1948_1970_R | TCGCAGTTCATCAGCACGAAGCG | 1075 |
| | | AACGC | | | | |
| 117 | TUFB_EC_757_774_F | AAGACGACCTGCACGGGC | 6 | TUFB_EC_849_867_R | GCGCTCCACGTCTTCACGC | 819 |
| 118 | 23S_EC_2646_2667_F | CTGTTCTTAGTACGAGAGG | 84 | 23S_EC_2745_2765_R | TTCGTGCTTAGATGCTTTCAG | 1389 |
| | | ACC | | | | |
| 119 | 16S_EC_969_985_1P_F | ACGCGAAGAACCTTACpC | 19 | 16S_EC_1061_1078_2P_R | ACGACACGAGCpTpGACGAC | 733 |
| 120 | 16S_EC_972_985_2P_F | CGAAGAACpCpTTACC | 63 | 16S_EC_1064_1075_2P_R | ACACGAGCpTpGAC | 727 |
| 121 | 16S_EC_972_985_F | CGAAGAACCTTACC | 63 | 16S_EC_1064_1075_R | ACACGAGCTGAC | 727 |
| 122 | TRNA_ILE- | CCTGATAAGGGTGAGGTCG | 61 | 23S_EC_40_59_R | ACGTCCTTCATCGCCTCTGA | 740 |
| | RRNH_EC_32_50.2_F | | | | | |
| 123 | 23S_EC_-7_15_F | GTTGTGAGGTTAAGCGACT | 140 | 23S_EC_430_450_R | CTATCGGTCAGTCAGGAGTAT | 799 |
| | | AAG | | | | |
| 124 | 23S_EC_-7_15_F | GTTGTGAGGTTAAGCGACT | 141 | 23S_EC_891_910_R | TTGCATCGGGTTGGTAAGTC | 1403 |
| | | AAG | | | | |
| 125 | 23S_EC_430_450_F | ATACTCCTGACTGACCGAT | 30 | 23S_EC_1424_1442_R | AACATAGCCTTCTCCGTCC | 712 |
| | | AG | | | | |
| 126 | 23S_EC_891_910_F | GACTTACCAACCCGATGCAA | 100 | 23S_EC_1908_1931_R | TACCTTAGGACCGTTATAGTTACG | 893 |
| 127 | 23S_EC_1424_1442_F | GGACGGAGAAGGCTATGTT | 117 | 23S_EC_2475_2494_R | CCAAACACCGCCGTCGATAT | 765 |
| 128 | 23S_EC_1908_1931_F | CGTAACTATAACGGTCCTA | 73 | 23S_EC_2833_2852_R | GCTTACACACCCGGCCTATC | 826 |
| | | AGGTA | | | | |
| 129 | 23S_EC_2475_2494_F | ATATCGACGGCGGTGTTTGG | 31 | TRNA_ASP- | GCGTGACAGGCAGGTATTC | 820 |
| | | | | RRNH_EC_23_41.2_R | | |
| 131 | 16S_EC_-60_- | AGTCTCAAGAGTGAACACG | 28 | 16S_EC_508_525_R | GCTGCTGGCACGGAGTTA | 823 |
| | 39_F | TAA | | | | |
| 132 | 16S_EC_326_345_F | GACACGGTCCAGACTCCTAC | 95 | 16S_EC_1041_1058_R | CCATGCAGCACCTGTCTC | 771 |
| 133 | 16S_EC_705_724_F | GATCTGGAGGAATACCGGTG | 107 | 16S_EC_1493_1512_R | ACGGTTACCTTGTTACGACT | 739 |
| 134 | 16S_EC_1268_1287_F | GAGAGCAAGCGGACCTCATA | 101 | TRNA_ALA- | CCTCCTGCGTGCAAAGC | 780 |
| | | | | RRNH_EC_30_46.2_R | | |
| 135 | 16S_EC_969_985_F | ACGCGAAGAACCTTACC | 19 | 16S_EC_1061_1078.2_R | ACAACACGAGCTGACGAC | 719 |
| 137 | 16S_EC_969_985_F | ACGCGAAGAACCTTACC | 19 | 16S_EC_1061_1078.2_I14_R | ACAACACGAGCTGICGAC | 721 |
| 138 | 16S_EC_969_985_F | ACGCGAAGAACCTTACC | 19 | 16S_EC_1061_1078.2_I12_R | ACAACACGAGCIGACGAC | 718 |
| 139 | 16S_EC_969_985_F | ACGCGAAGAACCTTACC | 19 | 16S_EC_1061_1078.2_I11_R | ACAACACGAGITGACGAC | 722 |
| 140 | 16S_EC_969_985_F | ACGCGAAGAACCTTACC | 19 | 16S_EC_1061_1078.2_I16_R | ACAACACGAGCTGACIAC | 720 |
| 141 | 16S_EC_969_985_F | ACGCGAAGAACCTTACC | 19 | 16S_EC_1061_1078.2_2I_R | ACAACACGAICTIACGAC | 723 |
| 142 | 16S_EC_969_985_F | ACGCGAAGAACCTTACC | 19 | 16S_EC_1061_1078.2_3I_R | ACAACACIAICTIACGAC | 724 |
| 143 | 16S_EC_969_985_F | ACGCGAAGAACCTTACC | 19 | 16S_EC_1061_1078.2_4I_R | ACAACACIAICTIACIAC | 725 |
| 147 | 23S_EC_2652_2669_F | CTAGTACGAGAGGACCGG | 79 | 23S_EC_2741_2760_R | ACTTAGATGCTTTCAGCGGT | 743 |
| 158 | 16S_EC_683_700_F | GTGTAGCGGTGAAATGCG | 137 | 16S_EC_880_894_R | CGTACTCCCCAGGCG | 796 |
| 159 | 16S_EC_1100_1116_F | CAACGAGCGCAACCCTT | 42 | 16S_EC_1174_1188_R | TCCCCACCTTCCTCC | 1019 |
| 215 | SSPE_BA_121_137_F | AACGCACAATCAGAAGC | 3 | SSPE_BA_197_216_R | TCTGTTTCAGTTGCAAATTC | 1132 |
| 220 | GROL_EC_941_959_F | TGGAAGATCTGGGTCAGGC | 544 | GROL_EC_1039_1060_R | CAATCTGCTGACGGATCTGAGC | 759 |
| 221 | INFB_EC_1103_1124_F | GTCGTGAAAACGAGCTGGA | 133 | INFB_EC_1174_1191_R | CATGATGGTCACAACCGG | 764 |
| | | AGA | | | | |
| 222 | HFLB_EC_1082_1102_F | TGGCGAACCTGGTGAACGA | 569 | HFLB_EC_1144_1168_R | CTTTCGCTTTCTCGAACTCAACC | 802 |
| | | AGC | | | AT | |
| 223 | INFB_EC_1969_1994_F | CGTCAGGGTAAATTCCGTG | 74 | INFB_EC_2038_2058_R | AACTTCGCCTTCGGTCATGTT | 713 |
| | | AAGTTAA | | | | |
| 224 | GROL_EC_219_242_F | GGTGAAAGAAGTTGCCTCT | 128 | GROL_EC_328_350_R | TTCAGGTCCATCGGGTTCATGCC | 1377 |
| | | AAAGC | | | | |
| 225 | VALS_EC_1105_1124_F | CGTGGCGGCGTGGTTATCGA | 77 | VALS_EC_1195_1214_R | ACGAACTGGATGTCGCCGTT | 732 |
| 226 | 16S_EC_556_575_F | CGGAATTACTGGGCGTAAAG | 70 | 16S_EC_683_700_R | CGCATTTCACCGCTACAC | 791 |
| 227 | RPOC_EC_1256_1277_F | ACCCAGTGCTGCTGAACCG | 16 | RPOC_EC_1295_1315_R | GTTCAAATGCCTGGATACCCA | 843 |
| | | TGC | | | | |
| 228 | 16S_EC_774_795_F | GGGAGCAAACAGGATTAGA | 122 | 16S_EC_880_894_R | CGTACTCCCCAGGCG | 796 |
| | | TAC | | | | |
| 229 | RPOC_EC_1584_1604_F | TGGCCCGAAAGAAGCTGAG | 567 | RPOC_EC_1623_1643_R | ACGCGGGCATGCAGAGATGCC | 737 |
| | | CG | | | | |
| 230 | 16S_EC_1082_1100_F | ATGTTGGGTTAAGTCCCGC | 37 | 16S_EC_1177_1196_R | TGACGTCATCCCCACCTTCC | 1158 |
| 231 | 16S_EC_1389_1407_F | CTTGTACACACCGCCCGTC | 88 | 16S_EC_1525_1541_R | AAGGAGGTGATCCAGCC | 714 |
| 232 | 16S_EC_1303_1323_F | CGGATTGGAGTCTGCAACT | 71 | 16S_EC_1389_1407_R | GACGGGCGGTGTGTACAAG | 808 |
| | | CG | | | | |
| 233 | 23S_EC_23_37_F | GGTGGATGCCTTGGC | 129 | 23S_EC_115_130_R | GGGTTTCCCCATTCGG | 833 |
| 234 | 23S_EC_187_207_F | GGGAACTGAAACATCTAAG | 121 | 23S_EC_242_256_R | TTCGCTCGCCGCTAC | 1385 |
| | | TA | | | | |
| 235 | 23S_EC_1602_1620_F | TACCCCAAACCGACACAGG | 184 | 23S_EC_1686_1703_R | CCTTCTCCCGAAGTTACG | 782 |
| 236 | 23S_EC_1685_1703_F | CCGTAACTTCGGGAGAAGG | 58 | 23S_EC_1828_1842_R | CACCGGGCAGGCGTC | 760 |
| 237 | 23S_EC_1827_1843_F | GACGCCTGCCCGGTGC | 99 | 23S_EC_1929_1949_R | CCGACAAGGAATTTCGCTACC | 775 |
| 238 | 23S_EC_2434_2456_F | AAGGTACTCCGGGGATAAC | 9 | 23S_EC_2490_2511_R | AGCCGACATCGAGGTGCCAAAC | 746 |
| | | AGGC | | | | |
| 239 | 23S_EC_2599_2616_F | GACAGTTCGGTCCCTATC | 96 | 23S_EC_2653_2669_R | CCGGTCCTCTCGTACTA | 777 |
| 240 | 23S_EC_2653_2669_F | TAGTACGAGAGGACCGG | 227 | 23S_EC_2737_2758_R | TTAGATGCTTTCAGCACTTATC | 1369 |
| 241 | 23S_BS_-68_- | AAACTAGATAACAGTAGAC | 1 | 23S_BS_5_21_R | GTGCGCCCTTTCTAACTT | 841 |
| | 44_F | ATCAC | | | | |
| 242 | 16S_EC_8_27_F | AGAGTTTGATCATGGCTCAG | 23 | 16S_EC_342_358_R | ACTGCTGCCTCCCGTAG | 742 |
| 243 | 16S_EC_314_332_F | CACTGGAACTGAGACACGG | 48 | 16S_EC_556_575_R | CTTTACGCCCAGTAATTCCG | 801 |
| 244 | 16S_EC_518_536_F | CCAGCAGCCGCGGTAATAC | 57 | 16S_EC_774_795_R | GTATCTAATCCTGTTTGCTCCC | 839 |
| 245 | 16S_EC_683_700_F | GTGTAGCGGTGAAATGCG | 137 | 16S_EC_967_985_R | GGTAAGGTTCTTCGCGTTG | 835 |
| 246 | 16S_EC_937_954_F | AAGCGGTGGAGCATGTGG | 7 | 16S_EC_1220_1240_R | ATTGTAGCACGTGTGTAGCCC | 757 |
| 247 | 16S_EC_1195_1213_F | CAAGTCATCATGGCCCTTA | 46 | 16S_EC_1525_1541_R | AAGGAGGTGATCCAGCC | 714 |
| 248 | 16S_EC_8_27_F | AGAGTTTGATCATGGCTCAG | 23 | 16S_EC_1525_1541_R | AAGGAGGTGATCCAGCC | 714 |
| 249 | 23S_EC_1831_1849_F | ACCTGCCCAGTGCTGGAAG | 18 | 23S_EC_1919_1936_R | TCGCTACCTTAGGACCGT | 1080 |
| 250 | 16S_EC_1387_1407_F | GCCTTGTACACACCTCCCG | 112 | 16S_EC_1494_1513_R | CACGGCTACCTTGTTACGAC | 761 |
| | | TC | | | | |
| 251 | 16S_EC_1390_1411_F | TTGTACACACCGCCCGTCA | 693 | 16S_EC_1486_1505_R | CCTTGTTACGACTTCACCCC | 783 |
| | | TAC | | | | |
| 252 | 16S_EC_1367_1387_F | TACGGTGAATACGTTCCCG | 191 | 16S_EC_1485_1506_R | ACCTTGTTACGACTTCACCCCA | 731 |
| | | GG | | | | |
| 253 | 16S_EC_804_822_F | ACCACGCCGTAAACGATGA | 14 | 16S_EC_909_929_R | CCCCCGTCAATTCCTTTGAGT | 773 |
| 254 | 16S_EC_791_812_F | GATACCCTGGTAGTCCACA | 106 | 16S_EC_886_904_R | GCCTTGCGACCGTACTCCC | 817 |
| | | CCG | | | | |
| 255 | 16S_EC_789_810_F | TAGATACCCTGGTAGTCCA | 206 | 16S_EC_882_899_R | GCGACCGTACTCCCCAGG | 818 |
| | | CGC | | | | |
| 256 | 16S_EC_1092_1109_F | TAGTCCCGCAACGAGCGC | 228 | 16S_EC_1174_1195_R | GACGTCATCCCCACCTTCCTCC | 810 |
| 257 | 23S_EC_2586_2607_F | TAGAACGTCGCGAGACAGT | 203 | 23S_EC_2658_2677_R | AGTCCATCCCGGTCCTCTCG | 749 |
| | | TCG | | | | |
| 258 | RNASEP_SA_31_49_F | GAGGAAAGTCCATGCTCAC | 103 | RNASEP_SA_358_379_R | ATAAGCCATGTTCTGTTCCATC | 750 |
| 258 | RNASEP_SA_31_49_F | GAGGAAAGTCCATGCTCAC | 103 | RNASEP_EC_345_362_R | ATAAGCCGGGTTCTGTCG | 751 |
| 258 | RNASEP_SA_31_49_F | GAGGAAAGTCCATGCTCAC | 103 | RNASEP_BS_363_384_R | GTAAGCCATGTTTTGTTCCATC | 838 |
| 258 | RNASEP_BS_43_61_F | GAGGAAAGTCCATGCTCGC | 104 | RNASEP_SA_358_379_R | ATAAGCCATGTTCTGTTCCATC | 750 |
| 258 | RNASEP_BS_43_61_F | GAGGAAAGTCCATGCTCGC | 104 | RNASEP_EC_345_362_R | ATAAGCCGGGTTCTGTCG | 751 |
| 258 | RNASEP_BS_43_61_F | GAGGAAAGTCCATGCTCGC | 104 | RNASEP_BS_363_384_R | GTAAGCCATGTTTTGTTCCATC | 838 |
| 258 | RNASEP_EC_61_77_F | GAGGAAAGTCCGGGCTC | 105 | RNASEP_SA_358_379_R | ATAAGCCATGTTCTGTTCCATC | 750 |
| 258 | RNASEP_EC_61_77_F | GAGGAAAGTCCGGGCTC | 105 | RNASEP_EC_345_362_R | ATAAGCCGGGTTCTGTCG | 751 |
| 258 | RNASEP_EC_61_77_F | GAGGAAAGTCCGGGCTC | 105 | RNASEP_BS_363_384_R | GTAAGCCATGTTTTGTTCCATC | 838 |
| 259 | RNASEP_BS_43_61_F | GAGGAAAGTCCATGCTCGC | 104 | RNASEP_BS_363_384_R | GTAAGCCATGTTTTGTTCCATC | 838 |
| 260 | RNASEP_EC_61_77_F | GAGGAAAGTCCGGGCTC | 105 | RNASEP_EC_345_362_R | ATAAGCCGGGTTCTGTCG | 751 |
| 262 | RNASEP_SA_31_49_F | GAGGAAAGTCCATGCTCAC | 103 | RNASEP_SA_358_379_R | ATAAGCCATGTTCTGTTCCATC | 750 |
| 263 | 16S_EC_1082_1100_F | ATGTTGGGTTAAGTCCCGC | 37 | 16S_EC_1525_1541_R | AAGGAGGTGATCCAGCC | 714 |
| 264 | 16S_EC_556_575_F | CGGAATTACTGGGCGTAAAG | 70 | 16S_EC_774_795_R | GTATCTAATCCTGTTTGCTCCC | 839 |
| 265 | 16S_EC_1082_1100_F | ATGTTGGGTTAAGTCCCGC | 37 | 16S_EC_1177_1196_10G_R | TGACGTCATGCCCACCTTCC | 1160 |
| 266 | 16S_EC_1082_1100_F | ATGTTGGGTTAAGTCCCGC | 37 | 16S_EC_1177_1196_10G_11G_R | TGACGTCATGGCCACCTTCC | 1161 |
| 268 | YAED_EC_513_532_F_MOD | GGTGTTAAATAGCCTGGCAG | 130 | TRNA_ALA- | AGACCTCCTGCGTGCAAAGC | 744 |
| | | | | RRNH_EC_30_49_F_MOD | | |
| 269 | 16S_EC_1082_1100_F_MOD | ATGTTGGGTTAAGTCCCGC | 37 | 16S_EC_1177_1196_R_MOD | TGACGTCATCCCCACCTTCC | 1158 |
| 270 | 23S_EC_2586_2607_F_MOD | TAGAACGTCGCGAGACAGT | 203 | 23S_EC_2658_2677_R_MOD | AGTCCATCCCGGTCCTCTCG | 749 |
| | | TCG | | | | |
| 272 | 16S_EC_969_985_F | ACGCGAAGAACCTTACC | 19 | 16S_EC_1389_1407_R | GACGGGCGGTGTGTACAAG | 807 |
| 273 | 16S_EC_683_700_F | GTGTAGCGGTGAAATGCG | 137 | 16S_EC_1303_1323_R | CGAGTTGCAGACTGCGATCCG | 788 |
| 274 | 16S_EC_49_68_F | TAACACATGCAAGTCGAACG | 152 | 16S_EC_880_894_R | CGTACTCCCCAGGCG | 796 |
| 275 | 16S_EC_49_68_F | TAACACATGCAAGTCGAACG | 152 | 16S_EC_1061_1078_R | ACGACACGAGCTGACGAC | 734 |
| 277 | CYA_BA_1349_1370_F | ACAACGAAGTACAATACAA | 12 | CYA_BA_1426_1447_R | CTTCTACATTTTTAGCCATCAC | 800 |
| | | GAC | | | | |
| 278 | 16S_EC_1090_1111_2_F | TTAAGTCCCGCAACGAGCG | 650 | 16S_EC_1175_1196_R | TGACGTCATCCCCACCTTCCTC | 1159 |
| | | CAA | | | | |
| 279 | 16S_EC_405 432_F | TGAGTGATGAAGGCCTTAG | 464 | 16S_EC_507_527_R | CGGCTGCTGGCACGAAGTTAG | 793 |
| | | GGTTGTAAA | | | | |
| 280 | GROL_EC_496_518_F | ATGGACAAGGTTGGCAAGG | 34 | GROL_EC_577_596_R | TAGCCGCGGTCGAATTGCAT | 914 |
| | | AAGG | | | | |
| 281 | GROL_EC_511_536_F | AAGGAAGGCGTGATCACCG | 8 | GROL_EC_571_593_R | CCGCGGTCGAATTGCATGCCTTC | 776 |
| | | TTGAAGA | | | | |
| 288 | RPOB_EC_3802_3821_F | CAGCGTTTCGGCGAAATGGA | 51 | RPOB_EC_3862_3885_R | CGACTTGACGGTTAACATTTCCTG | 786 |
| 289 | RPOB_EC_3799_3821_F | GGGCAGCGTTTCGGCGAAA | 124 | RPOB_EC_3862_3888_R | GTCCGACTTGACGGTCAACATTT | 840 |
| | | TGGA | | | CCTG | |
| 290 | RPOC_EC_2146_2174_F | CAGGAGTCGTTCAACTCGA | 52 | RPOC_EC_2227_2245_R | ACGCCATCAGGCCACGCAT | 736 |
| | | TCTACATGAT | | | | |
| 291 | ASPS_EC_405_422_F | GCACAACCTGCGGCTGCG | 110 | ASPS_EC_521_538_R | ACGGCACGAGGTAGTCGC | 738 |
| 292 | RPOC_EC_1374_1393_F | CGCCGACTTCGACGGTGACC | 69 | RPOC_EC_1437_1455_R | GAGCATCAGCGTGCGTGCT | 811 |
| 293 | TUFB_EC_957_979_F | CCACACGCCGTTCTTCAAC | 55 | TUFB_EC_1034_1058_R | GGCATCACCATTTCCTTGTCCTT | 829 |
| | | AACT | | | CG | |
| 294 | 16S_EC_7_33_F | GAGAGTTTGATCCTGGCTC | 102 | 16S_EC_101_122_R | TGTTACTCACCCGTCTGCCACT | 1345 |
| | | AGAACGAA | | | | |
| 295 | VALS_EC_610_649_F | ACCGAGCAAGGAGACCAGC | 17 | VALS_EC_705_727_R | TATAACGCACATCGTCAGGGTGA | 929 |
| 344 | 16S_EC_971_990_F | GCGAAGAACCTTACCAGGTC | 113 | 16S_EC_1043_1062_R | ACAACCATGCACCACCTGTC | 726 |
| 346 | 16S_EC_713_732_TMOD_F | TAGAACACCGATGGCGAAG | 202 | 16S_EC_789_809_TMOD_R | TCGTGGACTACCAGGGTATCTA | 1110 |
| | | GC | | | | |
| 347 | 16S_EC_785_806_TMOD_F | TGGATTAGAGACCCTGGTA | 560 | 16S_EC_880_897_TMOD_R | TGGCCGTACTCCCCAGGCG | 1278 |
| | | GTCC | | | | |
| 348 | 16S_EC_960_981_TMOD_F | TTTCGATGCAACGCGAAGA | 706 | 16S_EC_1054_1073_TMOD_R | TACGAGCTGACGACAGCCATG | 895 |
| | | ACCT | | | | |
| 349 | 23S_EC_1826_1843_TMOD_F | TCTGACACCTGCCCGGTGC | 401 | 23S_EC_1906_1924_TMOD_R | TGACCGTTATAGTTACGGCC | 1156 |
| 350 | CAPC_BA_274_303_TMOD_F | TGATTATTGTTATCCTGTT | 476 | CAPC_BA_349_376_TMOD_R | TGTAACCCTTGTCTTTGAATTGT | 1314 |
| | | ATGCCATTTGAG | | | ATTTGC | |
| 351 | CYA_BA_1353_1379_TMOD_F | TCGAAGTACAATACAAGAC | 355 | CYA_BA_1448_1467_TMOD_R | TTGTTAACGGCTTCAAGACCC | 1423 |
| | | AAAAGAAGG | | | | |
| 352 | INFB_EC_1365_1393_TMOD_F | TTGCTCGTGGTGCACAAGT | 687 | INFB_EC_1439_1467_TMOD_R | TTGCTGCTTTCGCATGGTTAATT | 1411 |
| | | AACGGATATTA | | | GCTTCAA | |
| 353 | LEF_BA_756_781_TMOD_F | TAGCTTTTGCATATTATAT | 220 | LEF_BA_843_872_TMOD_R | TTCTTCCAAGGATAGATTTATTT | 1394 |
| | | CGAGCCAC | | | CTTGTTCG | |
| 354 | RPOC_EC_2218_2241_TMOD_F | TCTGGCAGGTATGCGTGGT | 405 | RPOC_EC_2313_2337_TMOD_R | TCGCACCGTGGGTTGAGATGAAG | 1072 |
| | | CTGATG | | | TAC | |
| 355 | SSPE_BA_115_137_TMOD_F | TCAAGCAAACGCACAATCA | 255 | SSPE_BA_197_222_TMOD_R | TTGCACGTCTGTTTCAGTTGCAA | 1402 |
| | | GAAGC | | | ATTC | |
| 356 | RPLB_EC_650_679_TMOD_F | TGACCTACAGTAAGAGGTT | 449 | RPLB_EC_739_762_TMOD_R | TTCCAAGTGCTGGTTTACCCCAT | 1380 |
| | | CTGTAATGAACC | | | GG | |
| 357 | RPLB_EC_688_710_TMOD_F | TCATCCACACGGTGGTGGT | 296 | RPLB_EC_736_757_TMOD_R | TGTGCTGGTTTACCCCATGGAGT | 1337 |
| | | GAAGG | | | | |
| 358 | VALS_EC_1105_1124_TMOD_F | TCGTGGCGGCGTGGTTATC | 385 | VALS_EC_1195_1218_TMOD_R | TCGGTACGAACTGGATGTCGCCG | 1093 |
| | | GA | | | TT | |
| 359 | RPOB_EC_1845_1866_TMOD_F | TTATCGCTCAGGCGAACTC | 659 | RPOB_EC_1909_1929_TMOD_R | TGCTGGATTCGCCTTTGCTACG | 1250 |
| | | CAAC | | | | |
| 360 | 23S_EC_2646_2667_TMOD_F | TCTGTTCTTAGTACGAGAG | 409 | 23S_EC_2745_2765_TMOD_R | TTTCGTGCTTAGATGCTTTCAG | 1434 |
| | | GACC | | | | |
| 361 | 16S_EC_1090_1111_2_TMOD_F | TTTAAGTCCCGCAACGAGC | 697 | 16S_EC_1175_1196_TMOD_R | TTGACGTCATCCCCACCTTCCTC | 1398 |
| | | GCAA | | | | |
| 362 | RPOB_EC_3799_3821_TMOD_F | TGGGCAGCGTTTCGGCGAA | 581 | RPOB_EC_3862_3888_TMOD_R | TGTCCGACTTGACGGTCAACATT | 1325 |
| | | ATGGA | | | TCCTG | |
| 363 | RPOC_EC_2146_2174_TMOD_F | TCAGGAGTCGTTCAACTCG | 284 | RPOC_EC_2227_2245_TMOD_R | TACGCCATCAGGCCACGCAT | 898 |
| | | ATCTACATGAT | | | | |
| 364 | RPOC_EC_1374_1393_TMOD_F | TCGCCGACTTCGACGGTGA | 367 | RPOC_EC_1437_1455_TMOD_R | TGAGCATCAGCGTGCGTGCT | 1166 |
| | | CC | | | | |
| 367 | TUFB_EC_957_979_TMOD_F | TCCACACGCCGTTCTTCAA | 308 | TUFB_EC_1034_1058_TMOD_R | TGGCATCACCATTTCCTTGTCCT | 1276 |
| | | CAACT | | | TCG | |
| 423 | SP101_SPET11_893_921_TMOD_F | TGGGCAACAGCAGCGGATT | 580 | SP101_SPET11_988_1012_TMOD_R | TCATGACAGCCAAGACCTCACCC | 990 |
| | | GCGATTGCGCG | | | ACC | |
| 424 | SP101_SPET11_1154_1179_TMOD_F | TCAATACCGCAACAGCGGT | 258 | SP101_SPET11_1251_1277_TMOD_R | TGACCCCAACCTGGCCTTTTGTC | 1155 |
| | | GGCTTGGG | | | GTTGA | |
| 425 | SP101_SPET11_118_147_TMOD_F | TGCTGGTGAAAATAACCCA | 528 | SP101_SPET11_213_238_TMOD_R | TTGTGGCCGATTTCACCACCTGC | 1422 |
| | | GATGTCGTCTTC | | | TCCT | |
| 426 | SP101_SPET11_1314_1336_TMOD_F | TCGCAAAAAAATCCAGCTA | 363 | SP101_SPET11_1403_1431_TMOD_R | TAAACTATTTTTTTAGCTATACT | 849 |
| | | TTAGC | | | CGAACAC | |
| 427 | SP101_SPET11_1408_1437_TMOD_F | TCGAGTATAGCTAAAAAAA | 359 | SP101_SPET11_1486_1515_TMOD_R | TGGATAATTGGTCGTAACAAGGG | 1268 |
| | | TAGTTTATGACA | | | ATAGTGAG | |
| 428 | SP101_SPET11_1688_1716_TMOD_F | TCCTATATTAATCGTTTAC | 334 | SP101_SPET11_1783_1808_TMOD_R | TATATGATTATCATTGAACTGCG | 932 |
| | | AGAAACTGGCT | | | GCCG | |
| 429 | SP101_SPET11_1711_1733_TMOD_F | TCTGGCTAAAACTTTGGCA | 406 | SP101_SPET11_1808_1835_TMOD_R | TGCGTGACGACCTTCTTGAATTG | 1239 |
| | | ACGGT | | | TAATCA | |
| 430 | SP101_SPET11_1807_1835_TMOD_F | TATGATTACAATTCAAGAA | 235 | SP101_SPET11_1901_1927_TMOD_R | TTTGGACCTGTAATCAGCTGAAT | 1439 |
| | | GGTCGTCACGC | | | ACTGG | |
| 431 | SP101_SPET11_1967_1991_TMOD_F | TTAACGGTTATCATGGCCC | 649 | SP101_SPET11_2062_2083_TMOD_R | TATTGCCCAGAAATCAAATCATC | 940 |
| | | AGATGGG | | | | |
| 432 | SP101_SPET11_216_243_TMOD_F | TAGCAGGTGGTGAAATCGG | 210 | SP101_SPET11_308_333_TMOD_R | TTGCCACTTTGACAACTCCTGTT | 1404 |
| | | CCACATGATT | | | GCTG | |
| 433 | SP101_SPET11_2260_2283_TMOD_F | TCAGAGACCGTTTTATCCT | 272 | SP101_SPET11_2375_2397_TMOD_R | TTCTGGGTGACCTGGTGTTTTAGA | 1393 |
| | | ATCAGC | | | | |
| 434 | SP101_SPET11_2375_2399_TMOD_F | TTCTAAAACACCAGGTCAC | 675 | SP101_SPET11_2470_2497_TMOD_R | TAGCTGCTAGATGAGCTTCTGCC | 918 |
| | | CCAGAAG | | | ATGGCC | |
| 435 | SP101_SPET11_2468_2487_TMOD_F | TATGGCCATGGCAGAAGCT | 238 | SP101_SPET11_2543_2570_TMOD_R | TCCATAAGGTCACCGTCACCATT | 1007 |
| | | CA | | | CAAAGC | |
| 436 | SP101_SPET11_266_295_TMOD_F | TCTTGTACTTGTGGCTCAC | 417 | SP101_SPET11_355_380_TMOD_R | TGCTGCTTTGATGGCTGAATCCC | 1249 |
| | | ACGGCTGTTTGG | | | CTTC | |
| 437 | SP101_SPET11_2961_2984_TMOD_F | TACCATGACAGAAGGCATT | 183 | SP101_SPET11_3023_3045_TMOD_R | TGGAATTTACCAGCGATAGACACC | 1264 |
| | | TTGACA | | | | |
| 438 | SP101_SPET11_3075_3103_TMOD_F | TGATGACTTTTTAGCTAAT | 473 | SP101_SPET11_3168_3196_TMOD_R | TAATCGACGACCATCTTGGAAAG | 875 |
| | | GGTCAGGCAGC | | | ATTTCTC | |
| 439 | SP101_SPET11_322_344_TMOD_F | TGTCAAAGTGGCACGTTTA | 631 | SP101_SPET11_423_441_TMOD_R | TATCCCCTGCTTCTGCTGCC | 934 |
| | | CTGGC | | | | |
| 440 | SP101_SPET11_3386_3403_TMOD_F | TAGCGTAAAGGTGAACCTT | 215 | SP101_SPET11_3480_3506_TMOD_R | TCCAGCAGTTACTGTCCCCTCAT | 1005 |
| | | | | | CTTTG | |
| 441 | SP101_SPET11_3511_3535_TMOD_F | TGCTTCAGGAATCAATGAT | 531 | SP101_SPET11_3605_3629_TMOD_R | TGGGTCTACACCTGCACTTGCAT | 1294 |
| | | GGAGCAG | | | AAC | |
| 442 | SP101_SPET11_358_387_TMOD_F | TGGGGATTCAGCCATCAAA | 588 | SP101_SPET11_448_473_TMOD_R | TCCAACCTTTTCCACAACAGAAT | 998 |
| | | GCAGCTATTGAC | | | CAGC | |
| 443 | SP101_SPET11_600_629_TMOD_F | TCCTTACTTCGAACTATGA | 348 | SP101_SPET11_686_714_TMOD_R | TCCCATTTTTTCACGCATGCTGA | 1018 |
| | | ATCTTTTGGAAG | | | AAATATC | |
| 444 | SP101_SPET11_658_684_TMOD_F | TGGGGATTGATATCACCGA | 589 | SP101_SPET11_756_784_TMOD_R | TGATTGGCGATAAAGTGATATTT | 1189 |
| | | TAAGAAGAA | | | TCTAAAA | |
| 445 | SP101_SPET11_776_801_TMOD_F | TTCGCCAATCAAAACTAAG | 673 | SP101_SPET11_871_896_TMOD_R | TGCCCACCAGAAAGACTAGCAGG | 1217 |
| | | GGAATGGC | | | ATAA | |
| 446 | SP101_SPET11_1_29_TMOD_F | TAACCTTAATTGGAAAGAA | 154 | SP101_SPET11_92_116_TMOD_R | TCCTACCCAACGTTCACCAAGGG | 1044 |
| | | ACCCAAGAAGT | | | CAG | |
| 447 | SP101_SPET11_364_385_F | TCAGCCATCAAAGCAGCTA | 276 | SP101_SPET11_448_471_R | TACCTTTTCCACAACAGAATCAGC | 894 |
| | | TTG | | | | |
| 448 | SP101_SPET11_3085_3104_F | TAGCTAATGGTCAGGCAGCC | 216 | SP101_SPET11_3170_3194_R | TCGACGACCATCTTGGAAAGATT | 1066 |
| | | | | | TC | |
| 449 | RPLB_EC_690_710_F | TCCACACGGTGGTGGTGAA | 309 | RPLB_EC_737_758_R | TGTGCTGGTTTACCCCATGGAG | 1336 |
| | | GG | | | | |
| 481 | BONTA_X52066_538_552_F | TATGGCTCTACTCAA | 239 | BONTA_X52066_647_660_R | TGTTACTGCTGGAT | 1346 |
| 482 | BONTA_X52066_538_552P_F | TA*TpGGC*Tp*Cp*TpA* | 143 | BONTA_X52066_647_660P_R | TG*Tp*TpA*Cp*TpG*Cp*TpG | 1146 |
| | | Cp*Tp*CpAA | | | GAT | |
| 483 | BONTA_X52066_701_720_F | GAATAGCAATTAATCCAAAT | 94 | BONTA_X52066_759_775_R | TTACTTCTAACCCACTC | 1367 |
| 484 | BONTA_X52066_701_720P_F | GAA*TpAG*CpAA*Tp*Tp | 91 | BONTA_X52066_759_775P_R | TTA*Cp*Tp*Tp*Cp*TpAA*Cp | 1359 |
| | | AA*Tp*Cp*CpAAAT | | | *Cp*CpA*Cp*TpC | |
| 485 | BONTA_X52066_450_473_F | TCTAGTAATAATAGGACCC | 393 | BONTA_X52066_517_539_R | TAACCATTTCGCGTAAGATTCAA | 859 |
| | | TCAGC | | | | |
| 486 | BONTA_X52066_450_473P_F | T*Cp*TpAGTAATAATAGG | 142 | BONTA_X52066_517_539P_R | TAACCA*Tp*Tp*Tp*CpGCGTA | 857 |
| | | A*Cp*Cp*Cp*Tp*CpAGC | | | AGA*Tp*Tp*CpAA | |
| 487 | BONTA_X52066_591_620_F | TGAGTCACTTGAAGTTGAT | 463 | BONTA_X52066_644_671_R | TCATGTGCTAATGTTACTGCTGG | 992 |
| | | ACAAATCCTCT | | | ATCTG | |
| 608 | SSPE_BA_156_168P_F | TGGTpGCpTpAGCpATT | 616 | SSPE_BA_243_255P_R | TGCpAGCpTGATpTpGT | 1241 |
| 609 | SSPE_BA_75_89P_F | TACpAGAGTpTpTpGCpGAC | 192 | SSPE_BA_163_177P_R | TGTGCTpTpTpGAATpGCpT | 1338 |
| 610 | SSPE_BA_150_168P_F | TGCTTCTGGTpGCpTpAGC | 533 | SSPE_BA_243_264P_R | TGATTGTTTTGCpAGCpTGATpT | 1191 |
| | | pATT | | | pGT | |
| 611 | SSPE_BA_72_89P_F | TGGTACpAGAGTpTpTpGC | 602 | SSPE_BA_163_182P_R | TCATTTGTGCTpTpTpGAATpGC | 995 |
| | | pGAC | | | pT | |
| 612 | SSPE_BA_114_137P_F | TCAAGCAAACGCACAATpC | 255 | SSPE_BA_196_222P_R | TTGCACGTCpTpGTTTCAGTTGC | 1401 |
| | | pAGAAGC | | | AAATTC | |
| 699 | SSPE_BA_123_153_F | TGCACAATCAGAAGCTAAG | 488 | SSPE_BA_202_231_R | TTTCACAGCATGCACGTCTGTTT | 1431 |
| | | AAAGCGCAAGCT | | | CAGTTGC | |
| 700 | SSPE_BA_156_168_F | TGGTGCTAGCATT | 612 | SSPE_BA_243_255_R | TGCAGCTGATTGT | 1202 |
| 701 | SSPE_BA_75_89_F | TACAGAGTTTGCGAC | 179 | SSPE_BA_163_177_R | TGTGCTTTGAATGCT | 1338 |
| 702 | SSPE_BA_150_168_F | TGCTTCTGGTGCTAGCATT | 533 | SSPE_BA_243_264_R | TGATTGTTTTGCAGCTGATTGT | 1190 |
| 703 | SSPE_BA_72_89_F | TGGTACAGAGTTTGCGAC | 600 | SSPE_BA_163_182_R | TCATTTGTGCTTTGAATGCT | 995 |
| 704 | SSPE_BA_146_168_F | TGCAAGCTTCTGGTGCTAG | 484 | SSPE_BA_242_267_R | TTGTGATTGTTTTGCAGCTGATT | 1421 |
| | | CATT | | | GTG | |
| 705 | SSPE_BA_63_89_F | TGCTAGTTATGGTACAGAG | 518 | SSPE_BA_163_191_R | TCATAACTAGCATTTGTGCTTTG | 986 |
| | | TTTGCGAC | | | AATGCT | |
| 706 | SSPE_BA_114_137_F | TCAAGCAAACGCACAATCA | 255 | SSPE_BA_196_222_R | TTGCACGTCTGTTTCAGTTGCAA | 1402 |
| | | GAAGC | | | ATTC | |
| 770 | PLA_AF053945_7377_7402_F | TGACATCCGGCTCACGTTA | 442 | PLA_AF053945_7434_7462_R | TGTAAATTCCGCAAAGACTTTGG | 1313 |
| | | TTATGGT | | | CATTAG | |
| 771 | PLA_AF053945_7382_7404_F | TCCGGCTCACGTTATTATG | 327 | PLA_AF053945_7482_7502_R | TGGTCTGAGTACCTCCTTTGC | 1304 |
| | | GTAC | | | | |
| 772 | PLA_AF053945_7481_7503_F | TGCAAAGGAGGTACTCAGA | 481 | PLA_AF053945_7539_7562_R | TATTGGAAATACCGGCAGCATCTC | 943 |
| | | CCAT | | | | |
| 773 | PLA_AF053945_7186_7211_F | TTATACCGGAAACTTCCCG | 657 | PLA_AF053945_7257_7280_R | TAATGCGATACTGGCCTGCAAGTC | 879 |
| | | AAAGGAG | | | | |
| 774 | CAF1_AF053947_33407_33430_F | TCAGTTCCGTTATCGCCAT | 292 | CAF1_AF053947_33494_33514_R | TGCGGGCTGGTTCAACAAGAG | 1235 |
| | | TGCAT | | | | |
| 775 | CAF1_AF053947_33515_33541_F | TCACTCTTACATATAAGGA | 270 | CAF1_AF053947_33595_33621_R | TCCTGTTTTATAGCCGCCAAGAG | 1053 |
| | | AGGCGCTC | | | TAAG | |
| 776 | CAF1_AF053947_33435_33457_F | TGGAACTATTGCAACTGCT | 542 | CAF1_AF053947_33499_33517_R | TGATGCGGGCTGGTTCAAC | 1183 |
| | | AATG | | | | |
| 777 | CAF1_AF053947_33687_33716_F | TCAGGATGGAAATAACCAC | 286 | CAF1_AF053947_33755_33782_R | TCAAGGTTCTCACCGTTTACCTT | 962 |
| | | CAATTCACTAC | | | AGGAG | |
| 778 | INV_U22457_515_539_F | TGGCTCCTTGGTATGACTC | 573 | INV_U22457_571_598_R | TGTTAAGTGTGTTGCGGCTGTCT | 1343 |
| | | TGCTTC | | | TTATT | |
| 779 | INV_U22457_699_724_F | TGCTGAGGCCTGGACCGAT | 525 | INV_U22457_753_776_R | TCACGCGACGAGTGCCATCCATTG | 976 |
| | | TATTTAC | | | | |
| 780 | INV_U22457_834_858_F | TTATTTACCTGCACTCCCA | 664 | INV_U22457_942_966_R | TGACCCAAAGCTGAAAGCTTTAC | 1154 |
| | | CAACTG | | | TG | |
| 781 | INV_U22457_1558_1581_F | TGGTAACAGAGCCTTATAG | 597 | INV_U22457_1619_1643_R | TTGCGTTGCAGATTATCTTTACC | 1408 |
| | | GCGCA | | | AA | |
| 782 | LL_NC003143_2366996_2367019_F | TGTAGCCGCTAAGCACTAC | 627 | LL_NC003143_2367073_2367097_R | TCTCATCCCGATATTACCGCCAT | 1123 |
| | | CATCC | | | GA | |
| 783 | LL_NC003143_2367172_2367194_F | TGGACGGCATCACGATTCT | 550 | LL_NC003143_2367249_2367271_R | TGGCAACAGCTCAACACCTTTGG | 1272 |
| | | CTAC | | | | |
| 874 | RPLB_EC_649_679_F | TGICCIACIGTIIGIGGTT | 620 | RPLB_EC_739_762_TMOD_R | TTCCAAGTGCTGGTTTACCCCAT | 1380 |
| | | CTGTAATGAACC | | | GG | |
| 875 | RPLB_EC_642_679_P_F | TpCpCpTpTpGITpGICCI | 646 | RPLB_EC_739_762_TMOD_R | TTCCAAGTGCTGGTTTACCCCAT | 1380 |
| | | ACIGTIIGIGGTTCTGTAA | | | GG | |
| | | TGAACC | | | | |
| 876 | MECIA_Y14051_3315_3341_F | TTACACATATCGTGAGCAA | 653 | MECIA_Y14051_3367_3393_R | TGTGATATGGAGGTGTAGAAGGT | 1333 |
| | | TGAACTGA | | | GTTA | |
| 877 | MECA_Y14051_3774_3802_F | TAAAACAAACTACGGTAAC | 144 | MECA_Y14051_3828_3854_R | TCCCAATCTAACTTCCACATACC | 1015 |
| | | ATTGATCGCA | | | ATCT | |
| 878 | MECA_Y14051_3645_3670_F | TGAAGTAGAAATGACTGAA | 434 | MECA_Y14051_3690_3719_R | TGATCCTGAATGTTTATATCTTT | 1181 |
| | | CGTCCGA | | | AACGCCT | |
| 879 | MECA_Y14051_4507_4530_F | TCAGGTACTGCTATCCACC | 288 | MECA_Y14051_4555_4581_R | TGGATAGACGTCATATGAAGGTG | 1269 |
| | | CTCAA | | | TGCT | |
| 880 | MECA_Y14051_4510_4530_F | TGTACTGCTATCCACCCTC | 626 | MECA_Y14051_4586_4610_R | TATTCTTCGTTACTCATGCCATA | 939 |
| | | AA | | | CA | |
| 881 | MECA_Y14051_4669_4698_F | TCACCAGGTTCAACTCAAA | 262 | MECA_Y14051_4765_4793_R | TAACCACCCCAAGATTTATCTTT | 858 |
| | | AAATATTAACA | | | TTGCCA | |
| 882 | MECA_Y14051_4520_4530P_F | TCpCpACpCpCpTpCpAA | 389 | MECA_Y14051_4590_4600P_R | TpACpTpCpATpGCpCpA | 1357 |
| 883 | MECA_Y14051_4520_4530P_F | TCpCpACpCpCpTpCpAA | 389 | MECA_Y14051_4600_4610P_R | TpATpTpCpTpTpCpGTpT | 1358 |
| 902 | TRPE_AY094355_1467_1491_F | ATGTCGATTGCAATCCGTA | 36 | TRPE_AY094355_1569_1592_R | TGCGCGAGCTTTTATTTGGGTTTC | 1231 |
| | | CTTGTG | | | | |
| 903 | TRPE_AY094355_1445_1471_F | TGGATGGCATGGTGAAATG | 557 | TRPE_AY094355_1551_1580_R | TATTTGGGTTTCATTCCACTCAG | 944 |
| | | GATATGTC | | | ATTCTGG | |
| 904 | TRPE_AY094355_1278_1303_F | TCAAATGTACAAGGTGAAG | 247 | TRPE_AY094355_1392_1418_R | TCCTCTTTTCACAGGCTCTACTT | 1048 |
| | | TGCGTGA | | | CATC | |
| 905 | TRPE_AY094355_1064_1086_F | TCGACCTTTGGCAGGAACT | 357 | TRPE_AY094355_1171_1196_R | TACATCGTTTCGCCCAAGATCAA | 885 |
| | | AGAC | | | TCA | |
| 906 | TRPE_AY094355_666_688_F | GTGCATGCGGATACAGAGC | 135 | TRPE_AY094355_769_791_R | TTCAAAATGCGGAGGCGTATGTG | 1372 |
| | | AGAG | | | | |
| 907 | TRPE_AY094355_757_776_F | TGCAAGCGCGACCACATACG | 483 | TRPE_AY094355_864_883_R | TGCCCAGGTACAACCTGCAT | 1218 |
| 908 | RECA_AF251469_43_68_F | TGGTACATGTGCCTTCATT | 601 | RECA_AF251469_140_163_R | TTCAAGTGCTTGCTCACCATTGTC | 1375 |
| | | GATGCTG | | | | |
| 909 | RECA_AF251469_169_190_F | TGACATGCTTGTCCGTTCA | 446 | RECA_AF251469_277_300_R | TGGCTCATAAGACGCGCTTGTAGA | 1280 |
| | | GGC | | | | |
| 910 | PARC_X95819_87_110_F | TGGTGACTCGGCATGTTAT | 609 | PARC_X95819_201_222_R | TTCGGTATAACGCATCGCAGCA | 1387 |
| | | GAAGC | | | | |
| 911 | PARC_X95819_87_110_F | TGGTGACTCGGCATGTTAT | 609 | PARC_X95819_192_219_R | GGTATAACGCATCGCAGCAAAAG | 836 |
| | | GAAGC | | | ATTTA | |
| 912 | PARC_X95819_123_147_F | GGCTCAGCCATTTAGTTAC | 120 | PARC_X95819_232_260_R | TCGCTCAGCAATAATTCACTATA | 1081 |
| | | CGCTAT | | | AGCCGA | |
| 913 | PARC_X95819_43_63_F | TCAGCGCGTACAGTGGGTG | 277 | PARC_X95819_143_170_R | TTCCCCTGACCTTCGATTAAAGG | 1383 |
| | | AT | | | ATAGC | |
| 914 | OMPA_AY485227_272_301_F | TTACTCCATTATTGCTTGG | 655 | OMPA_AY485227_364_388_R | GAGCTGCGCCAACGAATAAATCG | 812 |
| | | TTACACTTTCC | | | TC | |
| 915 | OMPA_AY485227_379_401_F | TGCGCAGCTCTTGGTATCG | 509 | OMPA_AY485227_492_519_R | TGCCGTAACATAGAAGTTACCGT | 1223 |
| | | AGTT | | | TGATT | |
| 916 | OMPA_AY485227_311_335_F | TACACAACAATGGCGGTAA | 178 | OMPA_AY485227_424_453_R | TACGTCGCCTTTAACTTGGTTAT | 901 |
| | | AGATGG | | | ATTCAGC | |
| 917 | OMPA_AY485227_415_441_F | TGCCTCGAAGCTGAATATA | 506 | OMPA_AY485227_514_546_R | TCGGGCGTAGTTTTTAGTAATTA | 1092 |
| | | ACCAAGTT | | | AATCAGAAGT | |
| 918 | OMPA_AY485227_494_520_F | TCAACGGTAACTTCTATGT | 252 | OMPA_AY485227_569_596_R | TCGTCGTATTTATAGTGACCAGC | 1108 |
| | | TACTTCTG | | | ACCTA | |
| 919 | OMPA_AY485227_551_577_F | TCAAGCCGTACGTATTATT | 257 | OMPA_AY485227_658_680_R | TTTAAGCGCCAGAAAGCACCAAC | 1425 |
| | | AGGTGCTG | | | | |
| 920 | OMPA_AY485227_555_581_F | TCCGTACGTATTATTAGGT | 328 | OMPA_AY485227_635_662_R | TCAACACCAGCGTTACCTAAAGT | 954 |
| | | GCTGGTCA | | | ACCTT | |
| 921 | OMPA_AY485227_556_583_F | TCGTACGTATTATTAGGTG | 379 | OMPA_AY485227_659_683_R | TCGTTTAAGCGCCAGAAAGCACC | 1114 |
| | | CTGGTCACT | | | AA | |
| 922 | OMPA_AY485227_657_679_F | TGTTGGTGCTTTCTGGCGC | 645 | OMPA_AY485227_739_765_R | TAAGCCAGCAAGAGCTGTATAGT | 871 |
| | | TTAA | | | TCCA | |
| 923 | OMPA_AY485227_660_683_F | TGGTGCTTTCTGGCGCTTA | 613 | OMPA_AY485227_786_807_R | TACAGGAGCAGCAGGCTTCAAG | 884 |
| | | AACGA | | | | |
| 924 | GYRA_AF100557_4_23_F | TCTGCCCGTGTCGTTGGTGA | 402 | GYRA_AF100557_119_142_R | TCGAACCGAAGTTACCCTGACCAT | 1063 |
| 925 | GYRA_AF100557_70_94_F | TCCATTGTTCGTATGGCTC | 316 | GYRA_AF100557_178_201_R | TGCCAGCTTAGTCATACGGACTTC | 1211 |
| | | AAGACT | | | | |
| 926 | GYRB_AB008700_19_40_F | TCAGGTGGCTTACACGGCG | 289 | GYRB_AB008700_111_140_R | TATTGCGGATCACCATGATGATA | 941 |
| | | TAG | | | TTCTTGC | |
| 927 | GYRB_AB008700_265_292_F | TCTTTCTTGAATGCTGGTG | 420 | GYRB_AB008700_369_395_R | TCGTTGAGATGGTTTTTACCTTC | 1113 |
| | | TACGTATCG | | | GTTG | |
| 928 | GYRB_AB008700_368_394_F | TCAACGAAGGTAAAAACCA | 251 | GYRB_AB008700_466_494_R | TTTGTGAAACAGCGAACATTTTC | 1440 |
| | | TCTCAACG | | | TTGGTA | |
| 929 | GYRB_AB008700_477_504_F | TGTTCGCTGTTTCACAAAC | 641 | GYRB_AB008700_611_632_R | TCACGCGCATCATCACCAGTCA | 977 |
| | | AACATTCCA | | | | |
| 930 | GYRB_AB008700_760_787_F | TACTTACTTGAGAATCCAC | 198 | GYRB_AB008700_862_888_R | ACCTGCAATATCTAATGCACTCT | 729 |
| | | AAGCTGCAA | | | TACG | |
| 931 | WAAA_Z96925_2_29_F | TCTTGCTCTTTCGTGAGTT | 416 | WAAA_Z96925_115_138_R | CAAGCGGTTTGCCTCAAATAGTCA | 758 |
| | | CAGTAAATG | | | | |
| 932 | WAAA_Z96925_286_311_F | TCGATCTGGTTTCATGCTG | 360 | WAAA_Z96925_394_412_R | TGGCACGAGCCTGACCTGT | 1274 |
| | | TTTCAGT | | | | |
| 939 | RPOB_EC_3798_3821_F | TGGGCAGCGTTTCGGCGAA | 581 | RPOB_EC_3862_3889_R | TGTCCGACTTGACGGTCAGCATT | 1326 |
| | | ATGGA | | | TCCTG | |
| 940 | RPOB_EC_3798_3821_F | TGGGCAGCGTTTCGGCGAA | 581 | RPOB_EC_3862_3889_2_R | TGTCCGACTTGACGGTTAGCATT | 1327 |
| | | ATGGA | | | TCCTG | |
| 941 | TUFB_EC_275_299_F | TGATCACTGGTGCTGCTCA | 468 | TUFB_EC_337_362_R | TGGATGTGCTCACGAGTCTGTGG | 1271 |
| | | GATGGA | | | CAT | |
| 942 | TUFB_EC_251_278_F | TGCACGCCGACTATGTTAA | 493 | TUFB_EC_337_360_R | TATGTGCTCACGAGTTTGCGGCAT | 937 |
| | | GAACATGAT | | | | |
| 949 | GYRB_AB008700_760_787_F | TACTTACTTGAGAATCCAC | 198 | GYRB_AB008700_862_888_2_R | TCCTGCAATATCTAATGCACTCT | 1050 |
| | | AAGCTGCAA | | | TACG | |
| 958 | RPOC_EC_2223_2243_F | TGGTATGCGTGGTCTGATG | 605 | RPOC_EC_2329_2352_R | TGCTAGACCTTTACGTGCACCGTG | 1243 |
| | | GC | | | | |
| 959 | RPOC_EC_918_938_F | TCTGGATAACGGTCGTCGC | 404 | RPOC_EC_1009_1031_R | TCCAGCAGGTTCTGACGGAAACG | 1004 |
| | | GG | | | | |
| 960 | RPOC_EC_2334_2357_F | TGCTCGTAAGGGTCTGGCG | 523 | RPOC_EC_2380_2403_R | TACTAGACGACGGGTCAGGTAACC | 905 |
| | | GATAC | | | | |
| 961 | RPOC_EC_917_938_F | TATTGGACAACGGTCGTCG | 242 | RPOC_EC_1009_1034_R | TTACCGAGCAGGTTCTGACGGAA | 1362 |
| | | CGG | | | ACG | |
| 962 | RPOB_EC_2005_2027_F | TCGTTCCTGGAACACGATG | 387 | RPOB_EC_2041_2064_R | TTGACGTTGCATGTTCGAGCCCAT | 1399 |
| | | ACGC | | | | |
| 963 | RPOB_EC_1527_1549_F | TCAGCTGTCGCAGTTCATG | 282 | RPOB_EC_1630_1649_R | TCGTCGCGGACTTCGAAGCC | 1104 |
| | | GACC | | | | |
| 964 | INFB_EC_1347_1367_F | TGCGTTTACCGCAATGCGT | 515 | INFB_EC_1414_1432_R | TCGGCATCACGCCGTCGTC | 1090 |
| | | GC | | | | |
| 965 | VALS_EC_1128_1151_F | TATGCTGACCGACCAGTGG | 237 | VALS_EC_1231_1257_R | TTCGCGCATCCAGGAGAAGTACA | 1384 |
| | | TACGT | | | TGTT | |
| 978 | RPOC_EC_2145_2175_F | TCAGGAGTCGTTCAACTCG | 285 | RPOC_EC_2228_2247_R | TTACGCCATCAGGCCACGCA | 1363 |
| | | ATCTACATGATG | | | | |
| 1045 | CJST_CJ_1668_1700_F | TGCTCGAGTGATTGACTTT | 522 | CJST_CJ_1774_1799_R | TGAGCGTGTGGAAAAGGACTTGG | 1170 |
| | | GCTAAATTTAGAGA | | | ATG | |
| 1046 | CJST_CJ_2171_2197_F | TCGTTTGGTGGTGGTAGAT | 388 | CJST_CJ_2283_2313_R | TCTCTTTCAAAGCACCATTGCTC | 1126 |
| | | GAAAAAGG | | | ATTATAGT | |
| 1047 | CJST_CJ_584_616_F | TCCAGGACAAATGTATGAA | 315 | CJST_CJ_663_692_R | TTCATTTTCTGGTCCAAAGTAAG | 1379 |
| | | AAATGTCCAAGAAG | | | CAGTATC | |
| 1048 | CJST_CJ_360_394_F | TCCTGTTATCCCTGAAGTA | 346 | CJST_CJ_442_476_R | TCAACTGGTTCAAAAACATTAAG | 955 |
| | | GTTAATCAAGTTTGTT | | | TTGTAATTGTCC | |
| 1049 | CJST_CJ_2636_2668_F | TGCCTAGAAGATCTTAAAA | 504 | CJST_CJ_2753_2777_R | TTGCTGCCATAGCAAAGCCTACA | 1409 |
| | | ATTTCCGCCAACTT | | | GC | |
| 1050 | CJST_CJ_1290_1320_F | TGGCTTATCCAAATTTAGA | 575 | CJST_CJ_1406_1433_R | TTTGCTCATGATCTGCATGAAGC | 1437 |
| | | TCGTGGTTTTAC | | | ATAAA | |
| 1051 | CJST_CJ_3267_3293_F | TTTGATTTTACGCCGTCCT | 707 | CJST_CJ_3356_3385_R | TCAAAGAACCCGCACCTAATTCA | 951 |
| | | CCAGGTCG | | | TCATTTA | |
| 1052 | CJST_CJ_5_39_F | TAGGCGAAGATATACAAAG | 222 | CJST_CJ_104_137_R | TCCCTTATTTTTCTTTCTACTAC | 1029 |
| | | AGTATTAGAAGCTAGA | | | CTTCGGATAAT | |
| 1053 | CJST_CJ_1080_1110_F | TTGAGGGTATGCACCGTCT | 681 | CJST_CJ_1166_1198_R | TCCCCTCATGTTTAAATGATCAG | 1022 |
| | | TTTTGATTCTTT | | | GATAAAAAGC | |
| 1054 | CJST_CJ_2060_2090_F | TCCCGGACTTAATATCAAT | 323 | CJST_CJ_2148_2174_R | TCGATCCGCATCACCATCAAAAG | 1068 |
| | | GAAAATTGTGGA | | | CAAA | |
| 1055 | CJST_CJ_2869_2895_F | TGAAGCTTGTTCTTTAGCA | 432 | CJST_CJ_2979_3007_R | TCCTCCTTGTGCCTCAAAACGCA | 1045 |
| | | GGACTTCA | | | TTTTTA | |
| 1056 | CJST_CJ_1880_1910_F | TCCCAATTAATTCTGCCAT | 317 | CJST_CJ_1981_2011_R | TGGTTCTTACTTGCTTTGCATAA | 1309 |
| | | TTTTCCAGGTAT | | | ACTTTCCA | |
| 1057 | CJST_CJ_2185_2212_F | TAGATGAAAAGGGCGAAGT | 208 | CJST_CJ_2283_2316_R | TGAATTCTTTCAAAGCACCATTG | 1152 |
| | | GGCTAATGG | | | CTCATTATAGT | |
| 1058 | CJST_CJ_1643_1670_F | TTATCGTTTGTGGAGCTAG | 660 | CJST_CJ_1724_1752_R | TGCAATGTGTGCTATGTCAGCAA | 1198 |
| | | TGCTTATGC | | | AAAGAT | |
| 1059 | CJST_CJ_2165_2194_F | TGCGGATCGTTTGGTGGTT | 511 | CJST_CJ_2247_2278_R | TCCACACTGGATTGTAATTTACC | 1002 |
| | | GTAGATGAAAA | | | TTGTTCTTT | |
| 1060 | CJST_CJ_599_632_F | TGAAAAATGTCCAAGAAGC | 424 | CJST_CJ_711_743_R | TCCCGAACAATGAGTTGTATCAA | 1024 |
| | | ATAGCAAAAAAAGCA | | | CTATTTTTAC | |
| 1061 | CJST_CJ_360_393_F | TCCTGTTATCCCTGAAGTA | 345 | CJST_CJ_443_477_R | TACAACTGGTTCAAAAACATTAA | 882 |
| | | GTTAATCAAGTTTGT | | | GCTGTAATTGTC | |
| 1062 | CJST_CJ_2678_2703_F | TCCCCAGGACACCCTGAAA | 321 | CJST_CJ_2760_2787_R | TGTGCTTTTTTTGCTGCCATAGC | 1339 |
| | | TTTCAAC | | | AAAGC | |
| 1063 | CJST_CJ_1268_1299_F | AGTTATAAACACGGCTTTC | 29 | CJST_CJ_1349_1379_R | TCGGTTTAAGCTCTACATGATCG | 1096 |
| | | CTATGGCTTATCC | | | TAAGGATA | |
| 1064 | CJST_CJ_1680_1713_F | TGATTTTGCTAAATTTAGA | 479 | CJST_CJ_1795_1822_R | TATGTGTAGTTGAGCTTACTACA | 938 |
| | | GAAATTGCGGATGAA | | | TGAGC | |
| 1065 | CJST_CJ_2857_2887_F | TGGCATTTCTTATGAAGCT | 565 | CJST_CJ_2965_2998_R | TGCTTCAAAACGCATTTTTACAT | 1253 |
| | | TGTTCTTTAGCA | | | TTTCGTTAAAG | |
| 1070 | RNASEP_BKM_580_599_F | TGCGGGTAGGGAGCTTGAGC | 512 | RNASEP_BKM_665_686_R | TCCGATAAGCCGGATTCTGTGC | 1034 |
| 1071 | RNASEP_BKM_616_637_F | TCCTAGAGGAATGGCTGCC | 333 | RNASEP_BKM_665_687_R | TGCCGATAAGCCGGATTCTGTGC | 1222 |
| | | ACG | | | | |
| 1072 | RNASEP_BDP_574_592_F | TGGCACGGCCATCTCCGTG | 561 | RNASEP_BDP_616_635_R | TCGTTTCACCCTGTCATGCCG | 1115 |
| 1073 | 23S_BRM_1110_1129_F | TGCGCGGAAGATGTAACGGG | 510 | 23S_BRM_1176_1201_R | TCGCAGGCTTACAGAACGCTCTC | 1074 |
| | | | | | CTA | |
| 1074 | 23S_BRM_515_536_F | TGCATACAAACAGTCGGAG | 496 | 23S_BRM_616_635_R | TCGGACTCGCTTTCGCTACG | 1088 |
| | | CCT | | | | |
| 1075 | RNASEP_CLB_459_487_F | TAAGGATAGTGCAACAGAG | 162 | RNASEP_CLB_498_526_R | TGCTCTTACCTCACCGTTCCACC | 1247 |
| | | ATATACCGCC | | | CTTACC | |
| 1076 | RNASEP_CLB_459_487_F | TAAGGATAGTGCAACAGAG | 162 | RNASEP_CLB_498_522_R | TTTACCTCGCCTTTCCACCCTTA | 1426 |
| | | ATATACCGCC | | | CC | |
| 1077 | ICD_CXB_93_120_F | TCCTGACCGACCCATTATT | 343 | ICD_CXB_172_194_R | TAGGATTTTTCCACGGCGGCATC | 921 |
| | | CCCTTTATC | | | | |
| 1078 | ICD_CXB_92_120_F | TTCCTGACCGACCCATTAT | 671 | ICD_CXB_172_194_R | TAGGATTTTTCCACGGCGGCATC | 921 |
| | | TCCCTTTATC | | | | |
| 1079 | ICD_CXB_176_198_F | TCGCCGTGGAAAAATCCTA | 369 | ICD_CXB_224_247_R | TAGCCTTTTCTCCGGCGTAGATCT | 916 |
| | | CGCT | | | | |
| 1080 | IS1111A_NC002971_6866_6891_F | TCAGTATGTATCCACCGTA | 290 | IS1111A_NC002971_6928_6954_R | TAAACGTCCGATACCAATGGTTC | 848 |
| | | GCCAGTC | | | GCTC | |
| 1081 | IS1111A_NC002971_7456_7483_F | TGGGTGACATTCATCAATT | 594 | IS1111A_NC002971_7529_7554_R | TCAACAACACCTCCTTATTCCCA | 952 |
| | | TCATCGTTC | | | CTC | |
| 1082 | RNASEP_RKP_419_448_F | TGGTAAGAGCGCACCGGTA | 599 | RNASEP_RKP_542_565_R | TCAAGCGATCTACCCGCATTACAA | 957 |
| | | AGTTGGTAACA | | | | |
| 1083 | RNASEP_RKP_422_443_F | TAAGAGCGCACCGGTAAGT | 159 | RNASEP_RKP_542_565_R | TCAAGCGATCTACCCGCATTACAA | 957 |
| | | TGG | | | | |
| 1084 | RNASEP_RKP_466_491_F | TCCACCAAGAGCAAGATCA | 310 | RNASEP_RKP_542_565_R | TCAAGCGATCTACCCGCATTACAA | 957 |
| | | AATAGGC | | | | |
| 1085 | RNASEP_RKP_264_287_F | TCTAAATGGTCGTGCAGTT | 391 | RNASEP_RKP_295_321_R | TCTATAGAGTCCGGACTTTCCTC | 1119 |
| | | GCGTG | | | GTGA | |
| 1086 | RNASEP_RKP_426_448_F | TGCATACCGGTAAGTTGGC | 497 | RNASEP_RKP_542_565_R | TCAAGCGATCTACCCGCATTACAA | 957 |
| | | AACA | | | | |
| 1087 | OMPB_RKP_860_890_F | TTACAGGAAGTTTAGGTGG | 654 | OMPB_RKP_972_996_R | TCCTGCAGCTCTACCTGCTCCAT | 1051 |
| | | TAATCTAAAAGG | | | TA | |
| 1088 | OMPB_RKP_1192_1221_F | TCTACTGATTTTGGTAATC | 392 | OMPB_RKP_1288_1315_R | TAGCAgCAAAAGTTATCACACCT | 910 |
| | | TTGCAGCACAG | | | GCAGT | |
| 1089 | OMPB_RKP_3417_3440_F | TGCAAGTGGTACTTCAACA | 485 | OMPB_RKP_3520_3550_R | TGGTTGTAGTTCCTGTAGTTGTT | 1310 |
| | | TGGGG | | | GCATTAAC | |
| 1090 | GLTA_RKP_1043_1072_F | TGGGACTTGAAGCTATCGC | 576 | GLTA_RKP_1138_1162_R | TGAACATTTGCGACGGTATACCC | 1147 |
| | | TCTTAAAGATG | | | AT | |
| 1091 | GLTA_RKP_400_428_F | TCTTCTCATCCTATGGCTA | 413 | GLTA_RKP_499_529_R | TGGTGGGTATCTTAGCAATCATT | 1305 |
| | | TTATGCTTGC | | | CTAATAGC | |
| 1092 | GLTA_RKP_1023_1055_F | TCCGTTCTTACAAATAGCA | 330 | GLTA_RKP_1129_1156_R | TTGGCGACGGTATACCCATAGCT | 1415 |
| | | ATAGAACTTGAAGC | | | TTATA | |
| 1093 | GLTA_RKP_1043_1072_2_F | TGGAGCTTGAAGCTATCGC | 553 | GLTA_RKP_1138_1162_R | TGAACATTTGCGACGGTATACCC | 1147 |
| | | TCTTAAAGATG | | | AT | |
| 1094 | GLTA_RKP_1043_1072_3_F | TGGAACTTGAAGCTCTCGC | 543 | GLTA_RKP_1138_1164_R | TGTGAACATTTGCGACGGTATAC | 1330 |
| | | TCTTAAAGATG | | | CCAT | |
| 1095 | GLTA_RKP_400_428_F | TCTTCTCATCCTATGGCTA | 413 | GLTA_RKP_505_534_R | TGCGATGGTAGGTATCTTAGCAA | 1230 |
| | | TTATGCTTGC | | | TCATTCT | |
| 1096 | CTXA_VBC_117_142_F | TCTTATGCCAAGAGGACAG | 410 | CTXA_VBC_194_218_R | TGCCTAACAAATCCCGTCTGAGT | 1226 |
| | | AGTGAGT | | | TC | |
| 1097 | CTXA_VBC_351_377_F | TGTATTAGGGGCATACAGT | 630 | CTXA_VBC_441_466_R | TGTCATCAAGCACCCCAAAATGA | 1324 |
| | | CCTCATCC | | | ACT | |
| 1098 | RNASEP_VBC_331_349_F | TCCGCGGAGTTGACTGGGT | 325 | RNASEP_VBC_388_414_R | TGACTTTCCTCCCCCTTATCAGT | 1163 |
| | | | | | CTCC | |
| 1099 | TOXR_VBC_135_158_F | TCGATTAGGCAGCAACGAA | 362 | TOXR_VBC_221_246_R | TTCAAAACCTTGCTCTCGCCAAA | 1370 |
| | | AGCCG | | | CAA | |
| 1100 | ASD_FRT_1_29_F | TTGCTTAAAGTTGGTTTTA | 690 | ASD_FRT_86_116_R | TGAGATGTCGAAAAAAACGTTGG | 1164 |
| | | TTGGTTGGCG | | | CAAAATAC | |
| 1101 | ASD_FRT_43_76_F | TCAGTTTTAATGTCTCGTA | 295 | ASD_FRT_129_156_R | TCCATATTGTTGCATAAAACCTG | 1009 |
| | | TGATCGAATCAAAAG | | | TTGGC | |
| 1102 | GALE_FRT_168_199_F | TTATCAGCTAGACCTTTTA | 658 | GALE_FRT_241_269_R | TCACCTACAGCTTTAAAGCCAGC | 973 |
| | | GGTAAAGCTAAGC | | | AAAATG | |
| 1103 | GALE_FRT_834_865_F | TCAAAAAGCCCTAGGTAAA | 245 | GALE_FRT_901_925_R | TAGCCTTGGCAACATCAGCAAAA | 915 |
| | | GAGATTCCATATC | | | CT | |
| 1104 | GALE_FRT_308_339_F | TCCAAGGTACACTAAACTT | 306 | GALE_FRT_390_422_R | TCTTCTGTAAAGGGTGGTTTATT | 1136 |
| | | ACTTGAGCTAATG | | | ATTCATCCCA | |
| 1105 | IPAH_SGF_258_277_F | TGAGGACCGTGTCGCGCTCA | 458 | IPAH_SGF_301_327_R | TCCTTCTGATGCCTGATGGACCA | 1055 |
| | | | | | GGAG | |
| 1106 | IPAH_SGF_113_134_F | TCCTTGACCGCCTTTCCGA | 350 | IPAH_SGF_172_191_R | TTTTCCAGCCATGCAGCGAC | 1441 |
| | | TAC | | | | |
| 1107 | IPAH_SGF_462_486_F | TCAGACCATGCTCGCAGAG | 271 | IPAH_SGF_522_540_R | TGTCACTCCCGACACGCCA | 1322 |
| | | AAACTT | | | | |
| 1111 | RNASEP_BRM_461_488_F | TAAACCCCATCGGGAGCAA | 147 | RNASEP_BRM_542_561_R | TGCCTCGCGCAACCTACCCG | 1227 |
| | | GACCGAATA | | | | |
| 1112 | RNASEP_BRM_325_347_F | TACCCCAGGGAAAGTGCCA | 185 | RNASEP_BRM_402_428_R | TCTCTTACCCCACCCTTTCACCC | 1125 |
| | | CAGA | | | TTAC | |
| 1128 | HUPB_CJ_113_134_F | TAGTTGCTCAAACAGCTGG | 230 | HUPB_CJ_157_188_R | TCCCTAATAGTAGAAATAACTGC | 1028 |
| | | GCT | | | ATCAGTAGC | |
| 1129 | HUPB_CJ_76_102_F | TCCCGGAGCTTTTATGACT | 324 | HUPB_CJ_157_188_R | TCCCTAATAGTAGAAATAACTGC | 1028 |
| | | AAAGCAGAT | | | ATCAGTAGC | |
| 1130 | HUPB_CJ_76_102_F | TCCCGGAGCTTTTATGACT | 324 | HUPB_CJ_114_135_R | TAGCCCAGCTGTTTGAGCAACT | 913 |
| | | AAAGCAGAT | | | | |
| 1151 | AB_MLST-11- | TGAGATTGCTGAACATTTA | 454 | AB_MLST-11- | TTGTACATTTGAAACAATATGCA | 1418 |
| | OIF007_62_91_F | ATGCTGATTGA | | OIF007_169_203_R | TGACATGTGAAT | |
| 1152 | AB_MLST-11- | TATTGTTTCAAATGTACAA | 243 | AB_MLST-11- | TCACAGGTTCTACTTCATCAATA | 969 |
| | OIF007_185_214_F | GGTGAAGTGCG | | OIF007_291_324_R | ATTTCCATTGC | |
| 1153 | AB_MLST-11- | TGGAACGTTATCAGGTGCC | 541 | AB_MLST-11- | TTGCAATCGACATATCCATTTCA | 1400 |
| | OIF007_260_289_F | CCAAAAATTCG | | OIF007_364_393_R | CCATGCC | |
| 1154 | AB_MLST-11- | TGAAGTGCGTGATGATATC | 436 | AB_MLST-11- | TCCGCCAAAAACTCCCCTTTTCA | 1036 |
| | OIF007_206_239_F | GATGCACTTGATGTA | | OIF007_318_344_R | CAGG | |
| 1155 | AB_MLST-11- | TCGGTTTAGTAAAAGAACG | 378 | AB_MLST-11- | TTCTGCTTGAGGAATAGTGCGTGG | 1392 |
| | OIF007_522_552_F | TATTGCTCAACC | | OIF007_587_610_R | | |
| 1156 | AB_MLST-11- | TCAACCTGACTGCGTGAAT | 250 | AB_MLST-11- | TACGTTCTACGATTTCTTCATCA | 902 |
| | OIF007_547_571_F | GGTTGT | | OIF007_656_686_R | GGTACATC | |
| 1157 | AB_MLST-11- | TCAAGCAGAAGCTTTGGAA | 256 | AB_MLST-11- | TACAACGTGATAAACACGACCAG | 881 |
| | OIF007_601_627_F | GAAGAAGG | | OIF007_710_736_R | AAGC | |
| 1158 | AB_MLST-11- | TCGTGCCCGCAATTTGCAT | 384 | AB_MLST-11- | TAATGCCGGGTAGTGCAATCCAT | 878 |
| | OIF007_1202_1225_F | AAAGC | | OIF007_1266_1296_R | TCTTCTAG | |
| 1159 | AB_MLST-11- | TCGTGCCCGCAATTTGCAT | 384 | AB_MLST-11- | TGCACCTGCGGTCGAGCG | 1199 |
| | OIF007_1202_1225_F | AAAGC | | OIF007_1299_1316_R | | |
| 1160 | AB_MLST-11- | TTGTAGCACAGCAAGGCAA | 694 | AB_MLST-11- | TGCCATCCATAATCACGCCATAC | 1215 |
| | OIF007_1234_1264_F | ATTTCCTGAAAC | | OIF007_1335_1362_R | TGACG | |
| 1161 | AB_MLST-11- | TAGGTTTACGTCAGTATGG | 225 | AB_MLST-11- | TGCCAGTTTCCACATTTCACGTT | 1212 |
| | OIF007_1327_1356_F | CGTGATTATGG | | OIF007_1422_1448_R | CGTG | |
| 1162 | AB_MLST-11- | TCGTGATTATGGATGGCAA | 383 | AB_MLST-11- | TCGCTTGAGTGTAGTCATGATTG | 1083 |
| | OIF007_1345_1369_F | CGTGAA | | OIF007_1470_1494_R | CG | |
| 1163 | AB_MLST-11- | TTATGGATGGCAACGTGAA | 662 | AB_MLST-11- | TCGCTTGAGTGTAGTCATGATTG | 1083 |
| | OIF007_1351_1375_F | ACGCGT | | OIF007_1470_1494_R | CG | |
| 1164 | AB_MLST-11- | TCTTTGCCATTGAAGATGA | 422 | AB_MLST-11- | TCGCTTGAGTGTAGTCATGATTG | 1083 |
| | OIF007_1387_1412_F | CTTAAGC | | OIF007_1470_1494_R | CG | |
| 1165 | AB_MLST-11- | TACTAGCGGTAAGCTTAAA | 194 | AB_MLST-11- | TGAGTCGGGTTCACTTTACCTGG | 1173 |
| | OIF007_1542_1569_F | CAAGATTGC | | OIF007_1656_1680_R | CA | |
| 1166 | AB_MLST-11- | TTGCCAATGATATTCGTTG | 684 | AB_MLST-11- | TGAGTCGGGTTCACTTTACCTGG | 1173 |
| | OIF007_1566_1593_F | GTTAGCAAG | | OIF007_1656_1680_R | CA | |
| 1167 | AB_MLST-11- | TCGGCGAAATCCGTATTCC | 375 | AB_MLST-11- | TACCGGAAGCACCAGCGACATTA | 890 |
| | OIF007_1611_1638_F | TGAAAATGA | | OIF007_1731_1757_R | ATAG | |
| 1168 | AB_MLST-11- | TACCACTATTAATGTCGCT | 182 | AB_MLST-11- | TGCAACTGAATAGATTGCAGTAA | 1195 |
| | OIF007_1726_1752_F | GGTGCTTC | | OIF007_1790_1821_R | GTTATAAGC | |
| 1169 | AB_MLST-11- | TTATAACTTACTGCAATCT | 656 | AB_MLST-11- | TGAATTATGCAAGAAGTGATCAA | 1151 |
| | OIF007_1792_1826_F | ATTCAGTTGCTTGGTG | | OIF007_1876_1909_R | TTTTCTCACGA | |
| 1170 | AB_MLST-11- | TTATAACTTACTGCAATCT | 656 | AB_MLST-11- | TGCCGTAACTAACATAAGAGAAT | 1224 |
| | OIF007_1792_1826_F | ATTCAGTTGCTTGGTG | | OIF007_1895_1927_R | TATGCAAGAA | |
| 1171 | AB_MLST-11- | TGGTTATGTACCAAATACT | 618 | AB_MLST-11- | TGACGGCATCGATACCACCGTC | 1157 |
| | OIF007_1970_2002_F | TTGTCTGAAGATGG | | OIF007_2097_2118_R | | |
| 1172 | RNASEP_BRM_461_488_F | TAAACCCCATCGGGAGCAA | 147 | RNASEP_BRM_542_561_2_R | TGCCTCGTGCAACCCACCCG | 1228 |
| | | GACCGAATA | | | | |
| 2000 | CTXB_NC002505_46_70_F | TCAGCGTATGCACATGGAA | 278 | CTXB_NC002505_132_162_R | TCCGGCTAGAGATTCTGTATACG | 1039 |
| | | CTCCTC | | | ACAATATC | |
| 2001 | FUR_NC002505_87_113_F | TGAGTGCCAACATATCAGT | 465 | FUR_NC002505_205_228_R | TCCGCCTTCAAAATGGTGGCGAGT | 1037 |
| | | GCTGAAGA | | | | |
| 2002 | FUR_NC002505_87_113_F | TGAGTGCCAACATATCAGT | 465 | FUR_NC002505_178_205_R | TCACGATACCTGCATCATCAAAT | 974 |
| | | GCTGAAGA | | | TGGTT | |
| 2003 | GAPA_NC002505_533_560_F | TCGACAACACCATTATCTA | 356 | GAPA_NC002505_646_671_R | TCAGAATCGATGCCAAATGCGTC | 980 |
| | | TGGTGTGAA | | | ATC | |
| 2004 | GAPA_NC002505_694_721_F | TCAATGAACGACCAACAAG | 259 | GAPA_NC002505_769_798_R | TCCTCTATGCAACTTAGTATCAA | 1046 |
| | | TGATTGATG | | | CAGGAAT | |
| 2005 | GAPA_NC002505_753_782_F | TGCTAGTCAATCTATCATT | 517 | GAPA_NC002505_856_881_R | TCCATCGCAGTCACGTTTACTGT | 1011 |
| | | CCGGTTGATAC | | | TGG | |
| 2006 | GYRB_NC002505_2_32_F | TGCCGGACAATTACGATTC | 501 | GYRB_NC002505_109_134_R | TCCACCACCTCAAAGACCATGTG | 1003 |
| | | ATCGAGTATTAA | | | GTG | |
| 2007 | GYRB_NC002505_123_152_F | TGAGGTGGTGGATAACTCA | 460 | GYRB_NC002505_199_225_R | TCCGTCATCGCTGACAGAAACTG | 1042 |
| | | ATTGATGAAGC | | | AGTT | |
| 2008 | GYRB_NC002505_768_794_F | TATGCAGTGGAACGATGGT | 236 | GYRB_NC002505_832_860_R | TGGAAACCGGCTAAGTGAGTACC | 1262 |
| | | TTCCAAGA | | | ACCATC | |
| 2009 | GYRB_NC002505_837_860_F | TGGTACTCACTTAGCGGGT | 603 | GYRB_NC002505_937_957_R | TCCTTCACGCGCATCATCACC | 1054 |
| | | TTCCG | | | | |
| 2010 | GYRB_NC002505_934_956_F | TCGGGTGATGATGCGCGTG | 377 | GYRB_NC002505_982_1007_R | TGGCTTGAGAATTTAGGATCCGG | 1283 |
| | | AAGG | | | CAC | |
| 2011 | GYRB_NC002505_1161_1190_F | TAAAGCCCGTGAAATGACT | 148 | GYRB_NC002505_1255_1284_R | TGAGTCACCCTCCACAATGTATA | 1172 |
| | | CGTCGTAAAGG | | | GTTCAGA | |
| 2012 | OMPU_NC002505_85_110_F | TACGCTGACGGAATCAACC | 190 | OMPU_NC002505_154_180_R | TGCTTCAGCACGGCCACCAACTT | 1254 |
| | | AAAGCGG | | | CTAG | |
| 2013 | OMPU_NC002505_258_283_F | TGACGGCCTATACGGTGTT | 451 | OMPU_NC002505_346_369_R | TCCGAGACCAGCGTAGGTGTAACG | 1033 |
| | | GGTTTCT | | | | |
| 2014 | OMPU_NC002505_431_455_F | TCACCGATATCATGGCTTA | 266 | OMPU_NC002505_544_567_R | TCGGTCAGCAAAACGGTAGCTTGC | 1094 |
| | | CCACGG | | | | |
| 2015 | OMPU_NC002505_533_557_F | TAGGCGTGAAAGCAAGCTA | 223 | OMPU_NC002505_625_651_R | TAGAGAGTAGCCATCTTCACCGT | 908 |
| | | CCGTTT | | | TGTC | |
| 2016 | OMPU_NC002505_689_713_F | TAGGTGCTGGTTACGCAGA | 224 | OMPU_NC002505_725_751_R | TGGGGTAAGACGCGGCTAGCATG | 1291 |
| | | TCAAGA | | | TATT | |
| 2017 | OMPU_NC002505_727_747_F | TACATGCTAGCCGCGTCTT | 181 | OMPU_NC002505_811_835_R | TAGCAGCTAGCTCGTAACCAGTG | 911 |
| | | AC | | | TA | |
| 2018 | OMPU_NC002505_931_953_F | TACTACTTCAAGCCGAACT | 193 | OMPU_NC002505_1033_1053_R | TTAGAAGTCGTAACGTGGACC | 1368 |
| | | TCCG | | | | |
| 2019 | OMPU_NC002505_927_953_F | TACTTACTACTTCAAGCCG | 197 | OMPU_NC002505_1033_1054_R | TGGTTAGAAGTCGTAACGTGGACC | 1307 |
| | | AACTTCCG | | | | |
| 2020 | TCPA_NC002505_48_73_F | TCACGATAAGAAAACCGGT | 269 | TCPA_NC002505_148_170_R | TTCTGCGAATCAATCGCACGCTG | 1391 |
| | | CAAGAGG | | | | |
| 2021 | TDH_NC004605_265_289_F | TGGCTGACATCCTACATGA | 574 | TDH_NC004605_357_386_R | TGTTGAAGCTGTACTTGACCTGA | 1351 |
| | | CTGTGA | | | TTTTACG | |
| 2022 | VVHA_NC004460_772_802_F | TCTTATTCCAACTTCAAAC | 412 | VVHA_NC004460_862_886_R | TACCAAAGCGTGCACGATAGTTG | 887 |
| | | CGAACTATGACG | | | AG | |
| 2023 | 23S_EC_2643_2667_F | TGCCTGTTCTTAGTACGAG | 508 | 23S_EC_2746_2770_R | TGGGTTTCGCGCTTAGATGCTTT | 1297 |
| | | AGGACC | | | CA | |
| 2024 | 16S_EC_713_732_TMOD_F | TAGAACACCGATGGCGAAG | 202 | 16S_EC_789_811_R | TGCGTGGACTACCAGGGTATCTA | 1240 |
| | | GC | | | | |
| 2025 | 16S_EC_784_806_F | TGGATTAGAGACCCTGGTA | 560 | 16S_EC_880_897_TMOD_R | TGGCCGTACTCCCCAGGCG | 1278 |
| | | GTCC | | | | |
| 2026 | 16S_EC_959_981_F | TGTCGATGCAACGCGAAGA | 634 | 16S_EC_1052_1074_R | TACGAGCTGACGACAGCCATGCA | 896 |
| | | ACCT | | | | |
| 2027 | TUFB_EC_956_979_F | TGCACACGCCGTTCTTCAA | 489 | TUFB_EC_1034_1058_2_R | TGCATCACCATTTCCTTGTCCTT | 1204 |
| | | CAACT | | | CG | |
| 2028 | RPOC_EC_2146_2174_TMOD_F | TCAGGAGTCGTTCAACTCG | 284 | RPOC_EC_2227_2249_R | TGCTAGGCCATCAGGCCACGCAT | 1244 |
| | | ATCTACATGAT | | | | |
| 2029 | RPOB_EC_1841_1866_F | TGGTTATCGCTCAGGCGAA | 617 | RPOB_EC_1909_1929_TMOD_R | TGCTGGATTCGCCTTTGCTACG | 1250 |
| | | CTCCAAC | | | | |
| 2030 | RPLB_EC_650_679_TMOD_F | TGACCTACAGTAAGAGGTT | 449 | RPLB_EC_739_763_R | TGCCAAGTGCTGGTTTACCCCAT | 1208 |
| | | CTGTAATGAACC | | | GG | |
| 2031 | RPLB_EC_690_710_F | TCCACACGGTGGTGGTGAA | 309 | RPLB_EC_737_760_R | TGGGTGCTGGTTTACCCCATGGAG | 1295 |
| | | GG | | | | |
| 2032 | INFB_EC_1366_1393_F | TCTCGTGGTGCACAAGTAA | 397 | INFB_EC_1439_1469_R | TGTGCTGCTTTCGCATGGTTAAT | 1335 |
| | | CGGATATTA | | | TGCTTCAA | |
| 2033 | VALS_EC_1105_1124_TMOD_F | TCGTGGCGGCGTGGTTATC | 385 | VALS_EC_1195_1219_R | TGGGTACGAACTGGATGTCGCCG | 1292 |
| | | GA | | | TT | |
| 2034 | SSPE_BA_113_137_F | TGCAAGCAAACGCACAATC | 482 | SSPE_BA_197_222_TMOD_R | TTGCACGTCTGTTTCAGTTGCAA | 1402 |
| | | AGAAGC | | | ATTC | |
| 2035 | RPOC_EC_2218_2241_TMOD_F | TCTGGCAGGTATGCGTGGT | 405 | RPOC_EC_2313_2338_R | TGGCACCGTGGGTTGAGATGAAG | 1273 |
| | | CTGATG | | | TAC | |
| 2056 | MECI- | TTTACACATATCGTGAGCA | 698 | MECI-R_NC003923- | TTGTGATATGGAGGTGTAGAAGG | 1420 |
| | R_NC003923- | ATGAACTGA | | 41798-41609_86_113_R | TGTTA | |
| | 41798- | | | | | |
| | 41609_33_60_F | | | | | |
| 2057 | AGR- | TCACCAGTTTGCCACGTAT | 263 | AGR-III_NC003923- | ACCTGCATCCCTAAACGTACTTGC | 730 |
| | III_NC003923- | CTTCAA | | 2108074- | | |
| | 2108074- | | | 2109507_56_79_R | | |
| | 2109507_1_23_F | | | | | |
| 2058 | AGR- | TGAGCTTTTAGTTGACTTT | 457 | AGR-III_NC003923- | TACTTCAGCTTCGTCCAATAAAA | 906 |
| | III_NC003923- | TTCAACAGC | | 2108074- | AATCACAAT | |
| | 2108074- | | | 2109507_622_653_R | | |
| | 2109507_569_596_F | | | | | |
| 2059 | AGR- | TTTCACACAGCGTGTTTAT | 701 | AGR-III_NC003923- | TGTAGGCAAGTGCATAAGAAATT | 1319 |
| | III_NC003923- | AGTTCTACCA | | 2108074- | GATACA | |
| | 2108074- | | | 2109507_1070_1098_R | | |
| | 2109507_1024_1052_F | | | | | |
| 2060 | AGR- | TGGTGACTTCATAATGGAT | 610 | AGR- | TCCCCATTTAATAATTCCACCTA | 1021 |
| | I_AJ617706_622_651_F | GAAGTTGAAGT | | I_AJ617706_694_726_R | CTATCACACT | |
| 2061 | AGR- | TGGGATTTTAAAAAACATT | 579 | AGR- | TGGTACTTCAACTTCATCCATTA | 1302 |
| | I_AJ617706_580_611_F | GGTAACATCGCAG | | I_AJ617706_626_655_R | TGAAGTC | |
| 2062 | AGR- | TCTTGCAGCAGTTTATTTG | 415 | AGR-II_NC002745- | TTGTTTATTGTTTCCATATGCTA | 1424 |
| | II_NC002745- | ATGAACCTAAAGT | | 2079448- | CACACTTTC | |
| | 2079448- | | | 2080879_700_731_R | | |
| | 2080879_620_651_F | | | | | |
| 2063 | AGR- | TGTACCCGCTGAATTAACG | 624 | AGR-II_NC002745- | TCGCCATAGCTAAGTTGTTTATT | 1077 |
| | II_NC002745- | AATTTATACGAC | | 2079448- | GTTTCCAT | |
| | 2079448- | | | 2080879_715_745_R | | |
| | 2080879_649_679_F | | | | | |
| 2064 | AGR- | TGGTATTCTATTTTGCTGA | 606 | AGR- | TGCGCTATCAACGATTTTGACAA | 1233 |
| | IV_AJ617711_931_961_F | TAATGACCTCGC | | IV_AJ617711_1004_1035_R | TATATGTGA | |
| 2065 | AGR- | TGGCACTCTTGCCTTTAAT | 562 | AGR- | TCCCATACCTATGGCGATAACTG | 1017 |
| | IV_AJ617711_250_283_F | ATTAGTAAACTATCA | | IV_AJ617711_309_335_R | TCAT | |
| 2066 | BLAZ_NC002952 | TCCACTTATCGCAAATGGA | 312 | BLAZ_NC002952 | TGGCCACTTTTATCAGCAACCTT | 1277 |
| | (1913827 . . . 1914672)_68_68_F | AAATTAAGCAA | | (1913827 . . . 1914672)_68_68_R | ACAGTC | |
| 2067 | BLAZ_NC002952 | TGCACTTATCGCAAATGGA | 494 | BLAZ_NC002952 | TAGTCTTTTGGAACACCGTCTTT | 926 |
| | (1913827 . . . 1914672)_68_68_2_F | AAATTAAGCAA | | (1913827 . . . 1914672)_68_68_2_R | AATTAAAGT | |
| 2068 | BLAZ_NC002952 | TGATACTTCAACGCCTGCT | 467 | BLAZ_NC002952 | TGGAACACCGTCTTTAATTAAAG | 1263 |
| | (1913827 . . . 1914672)_68_68_3_F | GCTTTC | | (1913827 . . . 1914672)_68_68_3_R | TATCTCC | |
| 2069 | BLAZ_NC002952 | TATACTTCAACGCCTGCTG | 232 | BLAZ_NC002952 | TCTTTTCTTTGCTTAATTTTCCA | 1145 |
| | (1913827 . . . 1914672)_68_68_4_F | CTTTC | | (1913827 . . . 1914672)_68_68_4_R | TTTGCGAT | |
| 2070 | BLAZ_NC002952 | TGCAATTGCTTTAGTTTTA | 487 | BLAZ_NC002952 | TTACTTCCTTACCACTTTTAGTA | 1366 |
| | (1913827 . . . 1914672)_1_33_F | AGTGCATGTAATTC | | (1913827 . . . 1914672)_34_67_R | TCTAAAGCATA | |
| 2071 | BLAZ_NC002952 | TCCTTGCTTTAGTTTTAAG | 351 | BLAZ_NC002952 | TGGGGACTTCCTTACCACTTTTA | 1289 |
| | (1913827 . . . 1914672)_3_34_F | TGCATGTAATTCAA | | (1913827 . . . 1914672)_40_68_R | GTATCTAA | |
| 2072 | BSA-A_NC003923- | TAGCGAATGTGGCTTTACT | 214 | BSA-A_NC003923- | TGCAAGGGAAACCTAGAATTACA | 1197 |
| | 1304065- | TCACAATT | | 1304065- | AACCCT | |
| | 1303589_99_125_F | | | 1303589_165_193_R | | |
| 2073 | BSA-A_NC003923- | ATCAATTTGGTGGCCAAGA | 32 | BSA-A_NC003923- | TGCATAGGGAAGGTAACACCATA | 1203 |
| | 1304065- | ACCTGG | | 1304065- | GTT | |
| | 1303589_194_218_F | | | 1303589_253_278_R | | |
| 2074 | BSA-A_NC003923- | TTGACTGCGGCACAACACG | 679 | BSA-A_NC003923- | TAACAACGTTACCTTCGCGATCC | 856 |
| | 1304065- | GAT | | 1304065- | ACTAA | |
| | 1303589_328_349_F | | | 1303589_388_415_R | | |
| 2075 | BSA-A_NC003923- | TGCTATGGTGTTACCTTCC | 519 | BSA-A_NC003923- | TGTTGTGCCGCAGTCAAATATCT | 1353 |
| | 1304065- | CTATGCA | | 1304065- | AAATA | |
| | 1303589_253_278_F | | | 1303589_317_344_R | | |
| 2076 | BSA-B_NC003923- | TAGCAACAAATATATCTGA | 209 | BSA-B_NC003923- | TGTGAAGAACTTTCAAATCTGTG | 1331 |
| | 1917149- | AGCAGCGTACT | | 1917149- | AATCCA | |
| | 1914156_953_982_F | | | 1914156_1011_1039_R | | |
| 2077 | BSA-B_NC003923- | TGAAAAGTATGGATTTGAA | 426 | BSA-B_NC003923- | TCTTCTTGAAAAATTGTTGTCCC | 1138 |
| | 1917149- | CAACTCGTGAATA | | 1917149- | GAAAC | |
| | 1914156_1050_10_81_F | | | 1914156_1109_1136_R | | |
| 2078 | BSA-B_NC003923- | TCATTATCATGCGCCAATG | 300 | BSA-B_NC003923- | TGGACTAATAACAATGAGCTCAT | 1267 |
| | 1917149- | AGTGCAGA | | 1917149- | TGTACTGA | |
| | 1914156_1260_1286_F | | | 1914156_1323_1353_R | | |
| 2079 | BSA-B_NC003923- | TTTCATCTTATCGAGGACC | 703 | BSA-B_NC003923- | TGAATATGTAATGCAAACCAGTC | 1148 |
| | 1917149- | CGAAATCGA | | 1917149- | TTTGTCAT | |
| | 1914156_2126_2153_F | | | 1914156_2186_2216_R | | |
| 2080 | ERMA_NC002952- | TCGCTATCTTATCGTTGAG | 372 | ERMA_NC002952-55890- | TGAGTCTACACTTGGCTTAGGAT | 1174 |
| | 55890- | AAGGGATT | | 56621_487_513_R | GAAA | |
| | 56621_366_392_F | | | | | |
| 2081 | ERMA_NC002952- | TAGCTATCTTATCGTTGAG | 217 | ERMA_NC002952-55890- | TGAGCATTTTTATATCCATCTCC | 1167 |
| | 55890- | AAGGGATTTGC | | 56621_438_465_R | ACCAT | |
| | 56621_366_395_F | | | | | |
| 2082 | ERMA_NC002952- | TGATCGTTGAGAAGGGATT | 470 | ERMA_NC002952-55890- | TCTTGGCTTAGGATGAAAATATA | 1143 |
| | 55890- | TGCGAAAAGA | | 56621_473_504_R | GTGGTGGTA | |
| | 56621_374_402_F | | | | | |
| 2083 | ERMA_NC002952- | TGCAAAATCTGCAACGAGC | 480 | ERMA_NC002952-55890- | TCAATACAGAGTCTACACTTGGC | 964 |
| | 55890- | TTTGG | | 56621_491_520_R | TTAGGAT | |
| | 56621_404_427_F | | | | | |
| 2084 | ERMA_NC002952- | TCATCCTAAGCCAAGTGTA | 297 | ERMA_NC002952-55890- | TGGACGATATTCACGGTTTACCC | 1266 |
| | 55890- | GACTCTGTA | | 56621_586_615_R | ACTTATA | |
| | 56621_489_516_F | | | | | |
| 2085 | ERMA_NC002952- | TATAAGTGGGTAAACCGTG | 231 | ERMA_NC002952-55890- | TTGACATTTGCATGCTTCAAAGC | 1397 |
| | 55890- | AATATCGTGT | | 56621_640_665_R | CTG | |
| | 56621_586_614_F | | | | | |
| 2086 | ERMC_NC005908- | TCTGAACATGATAATATCT | 399 | ERMC_NC005908-2004- | TCCGTAGTTTTGCATAATTTATG | 1041 |
| | 2004- | TTGAAATCGGCTC | | 2738_173_206_R | GTCTATTTCAA | |
| | 2738_85_116_F | | | | | |
| 2087 | ERMC_NC005908- | TCATGATAATATCTTTGAA | 298 | ERMC_NC005908-2004- | TTTATGGTCTATTTCAATGGCAG | 1429 |
| | 2004- | ATCGGCTCAGGA | | 2738_160_189_R | TTACGAA | |
| | 2738_90_120_F | | | | | |
| 2088 | ERMC_NC005908- | TCAGGAAAAGGGCATTTTA | 283 | ERMC_NC005908-2004- | TATGGTCTATTTCAATGGCAGTT | 936 |
| | 2004- | CCCTTG | | 2738_161_187_R | ACGA | |
| | 2738_115_139_F | | | | | |
| 2089 | ERMC_NC005908- | TAATCGTGGAATACGGGTT | 168 | ERMC_NC005908-2004- | TCAACTTCTGCCATTAAAAGTAA | 956 |
| | 2004- | TGCTA | | 2738_425_452_R | TGCCA | |
| | 2738_374_397_F | | | | | |
| 2090 | ERMC_NC005908- | TCTTTGAAATCGGCTCAGG | 421 | ERMC_NC005908-2004- | TGATGGTCTATTTCAATGGCAGT | 1185 |
| | 2004- | AAAAGG | | 2738_159_188_R | TACGAAA | |
| | 2738_101_125_F | | | | | |
| 2091 | ERMB_Y13600- | TGTTGGGAGTATTCCTTAC | 644 | ERMB_Y13600-625- | TCAACAATCAGATAGATGTCAGA | 953 |
| | 625- | CATTTAAGCACA | | 1362_352_380_R | CGCATG | |
| | 1362_291_321_F | | | | | |
| 2092 | ERMB_Y13600- | TGGAAAGCCATGCGTCTGA | 536 | ERMB_Y13600-625- | TGCAAGAGCAACCCTAGTGTTCG | 1196 |
| | 625- | CATCT | | 1362_415_437_R | | |
| | 1362_344_367_F | | | | | |
| 2093 | ERMB_Y13600- | TGGATATTCACCGAACACT | 556 | ERMB_Y13600-625- | TAGGATGAAAGCATTCCGCTGGC | 919 |
| | 625- | AGGGTTG | | 1362_471_493_R | | |
| | 1362_404_429_F | | | | | |
| 2094 | ERMB_Y13600- | TAAGCTGCCAGCGGAATGC | 161 | ERMB_Y13600-625- | TCATCTGTGGTATGGCGGGTAAG | 989 |
| | 625- | TTTC | | 1362_521_545_R | TT | |
| | 1362_465_487_F | | | | | |
| 2095 | PVLUK_NC003923- | TGAGCTGCATCAACTGTAT | 456 | PVLUK_NC003923- | TGGAAAACTCATGAAATTAAAGT | 1261 |
| | 1529595- | TGGATAG | | 1529595- | GAAAGGA | |
| | 1531285_688_713_F | | | 1531285_775_804_R | | |
| 2096 | PVLUK_NC003923- | TGGAACAAAATAGTCTCTC | 539 | PVLUK_NC003923- | TCATTAGGTAAAATGTCTGGACA | 993 |
| | 1529595- | GGATTTTGACT | | 1529595- | TGATCCAA | |
| | 1531285_1039_1068_F | | | 1531285_1095_1125_R | | |
| 2097 | PVLUK_NC003923- | TGAGTAACATCCATATTTC | 461 | PVLUK_NC003923- | TCTCATGAAAAAGGCTCAGGAGA | 1124 |
| | 1529595- | TGCCATACGT | | 1529595- | TACAAG | |
| | 1531285_908_936_F | | | 1531285_950_978_R | | |
| 2098 | PVLUK_NC003923- | TCGGAATCTGATGTTGCAG | 373 | PVLUK_NC003923- | TCACACCTGTAAGTGAGAAAAAG | 968 |
| | 1529595- | TTGTT | | 1529595- | GTTGAT | |
| | 1531285_610_633_F | | | 1531285_654_682_R | | |
| 2099 | SA442_NC003923- | TGTCGGTACACGATATTCT | 635 | SA442_NC003923- | TTTCCGATGCAACGTAATGAGAT | 1433 |
| | 2538576- | TCACGA | | 2538576- | TTCA | |
| | 2538831_11_35_F | | | 2538831_98_124_R | | |
| 2100 | SA442_NC003923- | TGAAATCTCATTACGTTGC | 427 | SA442_NC003923- | TCGTATGACCAGCTTCGGTACTA | 1098 |
| | 2538576- | ATCGGAAA | | 2538576- | CTA | |
| | 2538831_98_124_F | | | 2538831_163_188_R | | |
| 2101 | SA442_NC003923- | TCTCATTACGTTGCATCGG | 395 | SA442_NC003923- | TTTATGACCAGCTTCGGTACTAC | 1428 |
| | 2538576- | AAACA | | 2538576- | TAAA | |
| | 2538831_103_126_F | | | 2538831_161_187_R | | |
| 2102 | SA442_NC003923- | TAGTACCGAAGCTGGTCAT | 226 | SA442_NC003923- | TGATAATGAAGGGAAACCTTTTT | 1179 |
| | 2538576- | ACGA | | 2538576- | CACG | |
| | 2538831_166_188_F | | | 2538831_231_257_R | | |
| 2103 | SEA_NC003923- | TGCAGGGAACAGCTTTAGG | 495 | SEA_NC003923- | TCGATCGTGACTCTCTTTATTTT | 1070 |
| | 2052219- | CA | | 2052219- | CAGTT | |
| | 2051456_115_135_F | | | 2051456_173_200_R | | |
| 2104 | SEA_NC003923- | TAACTCTGATGTTTTTGAT | 156 | SEA_NC003923- | TGTAATTAACCGAAGGTTCTGTA | 1315 |
| | 2052219- | GGGAAGGT | | 2052219- | GAAGTATG | |
| | 2051456_572_598_F | | | 2051456_621_651_R | | |
| 2105 | SEA_NC003923- | TGTATGGTGGTGTAACGTT | 629 | SEA_NC003923- | TAACCGTTTCCAAAGGTACTGTA | 861 |
| | 2052219- | ACATGATAATAATC | | 2052219- | TTTTGT | |
| | 2051456_382_414_F | | | 2051456_464_492_R | | |
| 2106 | SEA_NC003923- | TTGTATGTATGGTGGTGTA | 695 | SEA_NC003923- | TAACCGTTTCCAAAGGTACTGTA | 862 |
| | 2052219- | ACGTTACATGA | | 2052219- | TTTTGTTTACC | |
| | 2051456_377_406_F | | | 2051456_459_492_R | | |
| 2107 | SEB_NC002758- | TTTCACATGTAATTTTGAT | 702 | SEB_NC002758- | TCATCTGGTTTAGGATCTGGTTG | 988 |
| | 2135540- | ATTCGCACTGA | | 2135540- | ACT | |
| | 2135140_208_237_F | | | 2135140_273_298_R | | |
| 2108 | SEB_NC002758- | TATTTCACATGTAATTTTG | 244 | SEB_NC002758- | TGCAACTCATCTGGTTTAGGATCT | 1194 |
| | 2135540- | ATATTCGCACT | | 2135540- | | |
| | 2135140_206_235_F | | | 2135140_281_304_R | | |
| 2109 | SEB_NC002758- | TAACAACTCGCCTTATGAA | 151 | SEB_NC002758- | TGTGCAGGCATCATGTCATACCAA | 1334 |
| | 2135540- | ACGGGATATA | | 2135540- | | |
| | 2135140_402_402_F | | | 2135140_402_402_R | | |
| 2110 | SEB_NC002758- | TTGTATGTATGGTGGTGTA | 696 | SEB_NC002758- | TTACCATCTTCAAATACCCGAAC | 1361 |
| | 2135540- | ACTGAGCA | | 2135540- | AGTAA | |
| | 2135140_402_402_2_F | | | 2135140_402_402_2_R | | |
| 2111 | SEC_NC003923- | TTAACATGAAGGAAACCAC | 648 | SEC_NC003923-851678- | TGAGTTTGCACTTCAAAAGAAAT | 1177 |
| | 851678- | TTTGATAATGG | | 852768_620_647_R | TGTGT | |
| | 852768_546_575_F | | | | | |
| 2112 | SEC_NC003923- | TGGAATAACAAAACATGAA | 546 | SEC_NC003923-851678- | TCAGTTTGCACTTCAAAAGAAAT | 985 |
| | 851678- | GGAAACCACTT | | 852768_619_647_R | TGTGTT | |
| | 852768_537_566_F | | | | | |
| 2113 | SEC_NC003923- | TGAGTTTAACAGTTCACCA | 466 | SEC_NC003923-851678- | TCGCCTGGTGCAGGCATCATAT | 1078 |
| | 851678- | TATGAAACAGG | | 852768_794_815_R | | |
| | 852768_720_749_F | | | | | |
| 2114 | SEC_NC003923- | TGGTATGATATGATGCCTG | 604 | SEC_NC003923-851678- | TCTTCACACTTTTAGAATCAACC | 1133 |
| | 851678- | CACCA | | 852768_853_886_R | GTTTTATTGTC | |
| | 852768_787_810_F | | | | | |
| 2115 | SED_M28521_657_682_F | TGGTGGTGAAATAGATAGG | 615 | SED_M28521_741_770_R | TGTACACCATTTATCCACAAATT | 1318 |
| | | ACTGCTT | | | GATTGGT | |
| 2116 | SED_M28521_690_711_F | TGGAGGTGTCACTCCACAC | 554 | SED_M28521_739_770_R | TGGGCACCATTTATCCACAAATT | 1288 |
| | | GAA | | | GATTGGTAT | |
| 2117 | SED_M28521_833_854_F | TTGCACAAGCAAGGCGCTA | 683 | SED_M28521_888_911_R | TCGCGCTGTATTTTTCCTCCGAGA | 1079 |
| | | TTT | | | | |
| 2118 | SED_M28521_962_987_F | TGGATGTTAAGGGTGATTT | 559 | SED_M28521_1022_1048_R | TGTCAATATGAAGGTGCTCTGTG | 1320 |
| | | TCCCGAA | | | GATA | |
| 2119 | SEA- | TTTACACTACTTTTATTCA | 699 | SEA-SEE_NC002952- | TCATTTATTTCTTCGCTTTTCTC | 994 |
| | SEE_NC002952- | TTGCCCTAACG | | 2131289- | GCTAC | |
| | 2131289- | | | 2130703_71_98_R | | |
| | 2130703_16_45_F | | | | | |
| 2120 | SEA- | TGATCATCCGTGGTATAAC | 469 | SEA-SEE_NC002952- | TAAGCACCATATAAGTCTACTTT | 870 |
| | SEE_NC002952- | GATTTATTAGT | | 2131289- | TTTCCCTT | |
| | 2131289- | | | 2130703_314_344_R | | |
| | 2130703_249_278_F | | | | | |
| 2121 | SEE_NC002952- | TGACATGATAATAACCGAT | 445 | SEE_NC002952- | TCTATAGGTACTGTAGTTTGTTT | 1120 |
| | 2131289- | TGACCGAAGA | | 2131289- | TCCGTCT | |
| | 2130703_409_437_F | | | 2130703_465_494_R | | |
| 2122 | SEE_NC002952- | TGTTCAAGAGCTAGATCTT | 640 | SEE_NC002952- | TTTGCACCTTACCGCCAAAGCT | 1436 |
| | 2131289- | CAGGCAA | | 2131289- | | |
| | 2130703_525_550_F | | | 2130703_586_586_R | | |
| 2123 | SEE_NC002952- | TGTTCAAGAGCTAGATCTT | 639 | SEE_NC002952- | TACCTTACCGCCAAAGCTGTCT | 892 |
| | 2131289- | CAGGCA | | 2131289- | | |
| | 2130703_525_549_F | | | 2130703_586_586_2_R | | |
| 2124 | SEE_NC002952- | TCTGGAGGCACACCAAATA | 403 | SEE_NC002952- | TCCGTCTATCCACAAGTTAATTG | 1043 |
| | 2131289- | AAACA | | 2131289- | GTACT | |
| | 2130703_361_384_F | | | 2130703_444_471_R | | |
| 2125 | SEG_NC002758- | TGCTCAACCCGATCCTAAA | 520 | SEG_NC002758- | TAACTCCTCTTCCTTCAACAGGT | 863 |
| | 1955100- | TTAGACGA | | 1955100- | GGA | |
| | 1954171_225_251_F | | | 1954171_321_346_R | | |
| 2126 | SEG_NC002758- | TGGACAATAGACAATCACT | 548 | SEG_NC002758- | TGCTTTGTAATCTAGTTCCTGAA | 1260 |
| | 1955100- | TGGATTTACA | | 1955100- | TAGTAACCA | |
| | 1954171_623_651_F | | | 1954171_671_702_R | | |
| 2127 | SEG_NC002758- | TGGAGGTTGTTGTATGTAT | 555 | SEG_NC002758- | TGTCTATTGTCGATTGTTACCTG | 1329 |
| | 1955100- | GGTGGT | | 1955100- | TACAGT | |
| | 1954171_540_564_F | | | 1954171_607_635_R | | |
| 2128 | SEG_NC002758- | TACAAAGCAAGACACTGGC | 173 | SEG_NC002758- | TGATTCAAATGCAGAACCATCAA | 1187 |
| | 1955100- | TCACTA | | 1955100- | ACTCG | |
| | 1954171_694_718_F | | | 1954171_735_762_R | | |
| 2129 | SEH_NC002953- | TTGCAACTGCTGATTTAGC | 682 | SEH_NC002953-60024- | TAGTGTTGTACCTCCATATAGAC | 927 |
| | 60024- | TCAGA | | 60977_547_576_R | ATTCAGA | |
| | 60977_449_472_F | | | | | |
| 2130 | SEH_NC002953- | TAGAAATCAAGGTGATAGT | 201 | SEH_NC002953-60024- | TTCTGAGCTAAATCAGCAGTTGCA | 1390 |
| | 60024- | GGCAATGA | | 60977_450_473_R | | |
| | 60977_408_434_F | | | | | |
| 2131 | SEH_NC002953- | TCTGAATGTCTATATGGAG | 400 | SEH_NC002953-60024- | TACCATCTACCCAAACATTAGCA | 888 |
| | 60024- | GTACAACACTA | | 60977_608_634_R | CCAA | |
| | 60977_547_576_F | | | | | |
| 2132 | SEH_NC002953- | TTCTGAATGTCTATATGGA | 677 | SEH_NC002953-60024- | TAGCACCAATCACCCTTTCCTGT | 909 |
| | 60024- | GGTACAACACT | | 60977_594_616_R | | |
| | 60977_546_575_F | | | | | |
| 2133 | SEI_NC002758- | TCAACTCGAATTTTCAACA | 253 | SEI_NC002758- | TCACAAGGACCATTATAATCAAT | 966 |
| | 1957830- | GGTACCA | | 1957830- | GCCAA | |
| | 1956949_324_349_F | | | 1956949_419_446_R | | |
| 2134 | SEI_NC002758- | TTCAACAGGTACCAATGAT | 666 | SEI_NC002758- | TGTACAAGGACCATTATAATCAA | 1316 |
| | 1957830- | TTGATCTCA | | 1957830- | TGCCA | |
| | 1956949_336_363_F | | | 1956949_420_447_R | | |
| 2135 | SEI_NC002758- | TGATCTCAGAATCTAATAA | 471 | SEI_NC002758- | TCTGGCCCCTCCATACATGTATT | 1129 |
| | 1957830- | TTGGGACGAA | | 1957830- | TAG | |
| | 1956949_356_384_F | | | 1956949_449_474_R | | |
| 2136 | SEI_NC002758- | TCTCAAGGTGATATTGGTG | 394 | SEI_NC002758- | TGGGTAGGTTTTTATCTGTGACG | 1293 |
| | 1957830- | TAGGTAACTTAA | | 1957830- | CCTT | |
| | 1956949_223_253_F | | | 1956949_290_316_R | | |
| 2137 | SEJ_AF053140_1307_1332_F | TGTGGAGTAACACTGCATG | 637 | SEJ_AF053140_1381_1404_R | TCTAGCGGAACAACAGTTCTGATG | 1118 |
| | | AAAACAA | | | | |
| 2138 | SEJ_AF053140_1378_1403_F | TAGCATCAGAACTGTTGTT | 211 | SEJ_AF053140_1429_1458_R | TCCTGAAGATCTAGTTCTTGAAT | 1049 |
| | | CCGCTAG | | | GGTTACT | |
| 2139 | SEJ_AF053140_1431_1459_F | TAACCATTCAAGAACTAGA | 153 | SEJ_AF053140_1500_1531_R | TAGTCCTTTCTGAATTTTACCAT | 925 |
| | | TCTTCAGGCA | | | CAAAGGTAC | |
| 2140 | SEJ_AF053140_1434_1461_F | TCATTCAAGAACTAGATCT | 301 | SEJ_AF053140_1521_1549_R | TCAGGTATGAAACACGATTAGTC | 984 |
| | | TCAGGCAAG | | | CTTTCT | |
| 2141 | TSST_NC002758- | TGGTTTAGATAATTCCTTA | 619 | TSST_NC002758- | TGTAAAAGCAGGGCTATAATAAG | 1312 |
| | 2137564- | GGATCTATGCGT | | 2137564- | GACTC | |
| | 2138293_206_236_F | | | 2138293_278_305_R | | |
| 2142 | TSST_NC002758- | TGCGTATAAAAAACACAGA | 514 | TSST_NC002758- | TGCCCTTTTGTAAAAGCAGGGCT | 1221 |
| | 2137564- | TGGCAGCA | | 2137564- | AT | |
| | 2138293_232_258_F | | | 2138293_289_313_R | | |
| 2143 | TSST_NC002758- | TCCAAATAAGTGGCGTTAC | 304 | TSST_NC002758- | TACTTTAAGGGGCTATCTTTACC | 907 |
| | 2137564- | AAATACTGAA | | 2137564- | ATGAACCT | |
| | 2138293_382_410_F | | | 2138293_448_478_R | | |
| 2144 | TSST_NC002758- | TCTTTTACAAAAGGGGAAA | 423 | TSST_NC002758- | TAAGTTCCTTCGCTAGTATGTTG | 874 |
| | 2137564- | AAGTTGACTT | | 2137564- | GCTT | |
| | 2138293_297_325_F | | | 2138293_347_373_R | | |
| 2145 | ARCC_NC003923- | TCGCCGGCAATGCCATTGG | 368 | ARCC_NC003923- | TGAGTTAAAATGCGATTGATTTC | 1175 |
| | 2725050- | ATA | | 2725050- | AGTTTCCAA | |
| | 2724595_37_58_F | | | 2724595_97_128_R | | |
| 2146 | ARCC_NC003923- | TGAATAGTGATAGAACTGT | 437 | ARCC_NC003923- | TCTTCTTCTTTCGTATAAAAAGG | 1137 |
| | 2725050- | AGGCACAATCGT | | 2725050- | ACCAATTGG | |
| | 2724595_131_161_F | | | 2724595_214_245_R | | |
| 2147 | ARCC_NC003923- | TTGGTCCTTTTTATACGAA | 691 | ARCC_NC003923- | TGGTGTTCTAGTATAGATTGAGG | 1306 |
| | 2725050- | AGAAGAAGTTGAA | | 2725050- | TAGTGGTGA | |
| | 2724595_218_249_F | | | 2724595_322_353_R | | |
| 2148 | AROE_NC003923- | TTGCGAATAGAACGATGGC | 686 | AROE_NC003923- | TCGAATTCAGCTAAATACTTTTC | 1064 |
| | 1674726- | TCGT | | 1674726- | AGCATCT | |
| | 1674277_371_393_F | | | 1674277_435_464_R | | |
| 2149 | AROE_NC003923- | TGGGGCTTTAAATATTCCA | 590 | AROE_NC003923- | TACCTGCATTAATCGCTTGTTCA | 891 |
| | 1674726- | ATTGAAGATTTTCA | | 1674726- | TCAA | |
| | 1674277_30_62_F | | | 1674277_155_181_R | | |
| 2150 | AROE_NC003923- | TGATGGCAAGTGGATAGGG | 474 | AROE_NC003923- | TAAGCAATACCTTTACTTGCACC | 869 |
| | 1674726- | TATAATACAG | | 1674726- | ACCTG | |
| | 1674277_204_232_F | | | 1674277_308_335_R | | |
| 2151 | GLPF_NC003923- | TGCACCGGCTATTAAGAAT | 491 | GLPF_NC003923- | TGCAACAATTAATGCTCCGACAA | 1193 |
| | 1296927- | TACTTTGCCAACT | | 1296927- | TTAAAGGATT | |
| | 1297391_270_301_F | | | 1297391_382_414_R | | |
| 2152 | GLPF_NC003923- | TGGATGGGGATTAGCGGTT | 558 | GLPF_NC003923- | TAAAGACACCGCTGGGTTTAAAT | 850 |
| | 1296927- | ACAATG | | 1296927- | GTGCA | |
| | 1297391_27_51_F | | | 1297391_81_108_R | | |
| 2153 | GLPF_NC003923- | TAGCTGGCGCGAAATTAGG | 218 | GLPF_NC003923- | TCACCGATAAATAAAATACCTAA | 972 |
| | 1296927- | TGT | | 1296927- | AGTTAATGCCATTG | |
| | 1297391_239_260_F | | | 1297391_323_359_R | | |
| 2154 | GMK_NC003923- | TACTTTTTTAAAACTAGGG | 200 | GMK_NC003923- | TGATATTGAACTGGTGTACCATA | 1180 |
| | 1190906- | ATGCGTTTGAAGC | | 1190906- | ATAGTTGCC | |
| | 1191334_91_122_F | | | 1191334_166_197_R | | |
| 2155 | GMK_NC003923- | TGAAGTAGAAGGTGCAAAG | 435 | GMK_NC003923- | TCGCTCTCTCAAGTGATCTAAAC | 1082 |
| | 1190906- | CAAGTTAGA | | 1190906- | TTGGAG | |
| | 1191334_240_267_F | | | 1191334_305_333_R | | |
| 2156 | GMK_NC003923- | TCACCTCCAAGTTTAGATC | 268 | GMK_NC003923- | TGGGACGTAATCGTATAAATTCA | 1284 |
| | 1190906- | ACTTGAGAGA | | 1190906- | TCATTTC | |
| | 1191334_301_329_F | | | 1191334_403_432_R | | |
| 2157 | PTA_NC003923- | TCTTGTTTATGCTGGTAAA | 418 | PTA_NC003923-628885- | TGGTACACCTGGTTTCGTTTTGA | 1301 |
| | 628885- | GCAGATGG | | 629355_314_345_R | TGATTTGTA | |
| | 629355_237_263_F | | | | | |
| 2158 | PTA_NC003923- | TGAATTAGTTCAATCATTT | 439 | PTA_NC003923-628885- | TGCATTGTACCGAAGTAGTTCAC | 1207 |
| | 628885- | GTTGAACGACGT | | 629355_211_239_R | ATTGTT | |
| | 629355_141_171_F | | | | | |
| 2159 | PTA_NC003923- | TCCAAACCAGGTGTATCAA | 303 | PTA_NC003923-628885- | TGTTCTGGATTGATTGCACAATC | 1349 |
| | 628885- | GAACATCAGG | | 629355_393_422_R | ACCAAAG | |
| | 629355_328_356_F | | | | | |
| 2160 | TPI_NC003923- | TGCAAGTTAAGAAAGCTGT | 486 | TPI_NC003923-830671- | TGAGATGTTGATGATTTACCAGT | 1165 |
| | 830671- | TGCAGGTTTAT | | 831072_209_239_R | TCCGATTG | |
| | 831072_131_160_F | | | | | |
| 2161 | TPI_NC003923- | TCCCACGAAACAGATGAAG | 318 | TPI_NC003923-830671- | TGGTACAACATCGTTAGCTTTAC | 1300 |
| | 830671- | AAATTAACAAAAAAG | | 831072_97_129_R | CACTTTCACG | |
| | 831072_1_34_F | | | | | |
| 2162 | TPI_NC003923- | TCAAACTGGGCAATCGGAA | 246 | TPI_NC003923-830671- | TGGCAGCAATAGTTTGACGTACA | 1275 |
| | 830671- | CTGGTAAATC | | 831072_253_286_R | AATGCACACAT | |
| | 831072_199_227_F | | | | | |
| 2163 | YQI_NC003923- | TGAATTGCTGCTATGAAAG | 440 | YQI_NC003923-378916- | TCGCCAGCTAGCACGATGTCATT | 1076 |
| | 378916- | GTGGCTT | | 379431_259_284_R | TTC | |
| | 379431_142_167_F | | | | | |
| 2164 | YQI_NC003923- | TACAACATATTATTAAAGA | 175 | YQI_NC003923-378916- | TTCGTGCTGGATTTTGTCCTTGT | 1388 |
| | 378916- | GACGGGTTTGAATCC | | 379431_120_145_R | CCT | |
| | 379431_44_77_F | | | | | |
| 2165 | YQI_NC003923- | TCCAGCACGAATTGCTGCT | 314 | YQI_NC003923-378916- | TCCAACCCAGAACCACATACTTT | 997 |
| | 378916- | ATGAAAG | | 379431_193_221_R | ATTCAC | |
| | 379431_135_160_F | | | | | |
| 2166 | YQI_NC003923- | TAGCTGGCGGTATGGAGAA | 219 | YQI_NC003923-378916- | TCCATCTGTTAAACCATCATATA | 1013 |
| | 378916- | TATGTCT | | 379431_364_396_R | CCATGCTATC | |
| | 379431_275_300_F | | | | | |
| 2167 | BLAZ_(1913827 . . . 1914672)_546_575_F | TCCACTTATCGCAAATGGA | 312 | BLAZ_(1913827 . . . 1914672)_655_683_R | TGGCCACTTTTATCAGCAACCTT | 1277 |
| | | AAATTAAGCAA | | | ACAGTC | |
| 2168 | BLAZ_(1913827 . . . 1914672)_546_575_2_F | TGCACTTATCGCAAATGGA | 494 | BLAZ_(1913827 . . . 1914672)_628_659_R | TAGTCTTTTGGAACACCGTCTTT | 926 |
| | | AAATTAAGCAA | | | AATTAAAGT | |
| 2169 | BLAZ_(1913827 . . . 1914672)_507_531_F | TGATACTTCAACGCCTGCT | 467 | BLAZ_(1913827 . . . 1914672)_622_651_R | TGGAACACCGTCTTTAATTAAAG | 1263 |
| | | GCTTTC | | | TATCTCC | |
| 2170 | BLAZ_(1913827 . . . 1914672)_508_531_F | TATACTTCAACGCCTGCTG | 232 | BLAZ_(1913827 . . . 1914672)_553_583_R | TCTTTTCTTTGCTTAATTTTCCA | 1145 |
| | | CTTTC | | | TTTGCGAT | |
| 2171 | BLAZ_(1913827 . . . 1914672)_24_56_F | TGCAATTGCTTTAGTTTTA | 487 | BLAZ_(1913827 . . . 1914672)_121_154_R | TTACTTCCTTACCACTTTTAGTA | 1366 |
| | | AGTGCATGTAATTC | | | TCTAAAGCATA | |
| 2172 | BLAZ_(1913827 . . . 1914672)_26_58_F | TCCTTGCTTTAGTTTTAAG | 351 | BLAZ_(1913827 . . . 1914672)_127_157_R | TGGGGACTTCCTTACCACTTTTA | 1289 |
| | | TGCATGTAATTCAA | | | GTATCTAA | |
| 2173 | BLAZ_NC002952- | TCCACTTATCGCAAATGGA | 312 | BLAZ_NC002952- | TGGCCACTTTTATCAGCAACCTT | 1277 |
| | 1913827- | AAATTAAGCAA | | 1913827- | ACAGTC | |
| | 1914672_546_575_F | | | 1914672_655_683_R | | |
| 2174 | BLAZ_NC002952- | TGCACTTATCGCAAATGGA | 494 | BLAZ_NC002952- | TAGTCTTTTGGAACACCGTCTTT | 926 |
| | 1913827- | AAATTAAGCAA | | 1913827- | AATTAAAGT | |
| | 1914672_546_575_2_F | | | 1914672_628_659_R | | |
| 2175 | BLAZ_NC002952- | TGATACTTCAACGCCTGCT | 467 | BLAZ_NC002952- | TGGAACACCGTCTTTAATTAAAG | 1263 |
| | 1913827- | GCTTTC | | 1913827- | TATCTCC | |
| | 1914672_507_531_F | | | 1914672_622_651_R | | |
| 2176 | BLAZ_NC002952- | TATACTTCAACGCCTGCTG | 232 | BLAZ_NC002952- | TCTTTTCTTTGCTTAATTTTCCA | 1145 |
| | 1913827- | CTTTC | | 1913827- | TTTGCGAT | |
| | 1914672_508_531_F | | | 1914672_553_583_R | | |
| 2177 | BLAZ_NC002952- | TGCAATTGCTTTAGTTTTA | 487 | BLAZ_NC002952- | TTACTTCCTTACCACTTTTAGTA | 1366 |
| | 1913827- | AGTGCATGTAATTC | | 1913827- | TCTAAAGCATA | |
| | 1914672_24_56_F | | | 1914672_121_154_R | | |
| 2178 | BLAZ_NC002952- | TCCTTGCTTTAGTTTTAAG | 351 | BLAZ_NC002952- | TGGGGACTTCCTTACCACTTTTA | 1289 |
| | 1913827- | TGCATGTAATTCAA | | 1913827- | GTATCTAA | |
| | 1914672_26_58_F | | | 1914672_127_157_R | | |
| 2247 | TUFB_NC002758- | TGTTGAACGTGGTCAAATC | 643 | TUFB_NC002758- | TGTCACCAGCTTCAGCGTAGTCT | 1321 |
| | 615038- | AAAGTTGGTG | | 615038- | AATAA | |
| | 616222_693_721_F | | | 616222_793_820_R | | |
| 2248 | TUFB_NC002758- | TCGTGTTGAACGTGGTCAA | 386 | TUFB_NC002758- | TGTCACCAGCTTCAGCGTAGTCT | 1321 |
| | 615038- | ATCAAAGT | | 615038- | AATAA | |
| | 616222_690_716_F | | | 616222_793_820_R | | |
| 2249 | TUFB_NC002758- | TGAACGTGGTCAAATCAAA | 430 | TUFB_NC002758- | TGTCACCAGCTTCAGCGTAGTCT | 1321 |
| | 615038- | GTTGGTGAAGA | | 615038- | AATAA | |
| | 616222_696_725_F | | | 616222_793_820_R | | |
| 2250 | TUFB_NC002758- | TCCCAGGTGACGATGTACC | 320 | TUFB_NC002758- | TGGTTTGTCAGAATCACGTTCTG | 1311 |
| | 615038- | TGTAATC | | 615038- | GAGTTGG | |
| | 616222_488_513_F | | | 616222_601_630_R | | |
| 2251 | TUFB_NC002758- | TGAAGGTGGACGTCACACT | 433 | TUFB_NC002758- | TAGGCATAACCATTTCAGTACCT | 922 |
| | 615038- | CCATTCTTC | | 615038- | TCTGGTAA | |
| | 616222_945_972_F | | | 616222_1030_1060_R | | |
| 2252 | TUFB_NC002758- | TCCAATGCCACAAACTCGT | 307 | TUFB_NC002758- | TTCCATTTCAACTAATTCTAATA | 1382 |
| | 615038- | GAACA | | 615038- | ATTCTTCATCGTC | |
| | 616222_333_356_F | | | 616222_424_459_R | | |
| 2253 | NUC_NC002758- | TCCTGAAGCAAGTGCATTT | 342 | NUC_NC002758-894288- | TACGCTAAGCCACGTCCATATTT | 899 |
| | 894288- | ACGA | | 894974_483_509_R | ATCA | |
| | 894974_402_424_F | | | | | |
| 2254 | NUC_NC002758- | TCCTTATAGGGATGGCTAT | 349 | NUC_NC002758-894288- | TGTTTGTGATGCATTTGCTGAGC | 1354 |
| | 894288- | CAGTAATGTT | | 894974_165_189_R | TA | |
| | 894974_53_81_F | | | | | |
| 2255 | NUC_NC002758- | TCAGCAAATGCATCACAAA | 273 | NUC_NC002758-894288- | TAGTTGAAGTTGCACTATATACT | 928 |
| | 894288- | CAGATAA | | 894974_222_250_R | GTTGGA | |
| | 894974_169_194_F | | | | | |
| 2256 | NUC_NC002758- | TACAAAGGTCAACCAATGA | 174 | NUC_NC002758-894288- | TAAATGCACTTGCTTCAGGGCCA | 853 |
| | 894288- | CATTCAGACTA | | 894974_396_421_R | TAT | |
| | 894974_316_345_F | | | | | |
| 2270 | RPOB_EC_3798_3821_1_F | TGGCCAGCGCTTCGGTGAA | 566 | RPOB_EC_3868_3895_R | TCACGTCGTCCGACTTCACGGTC | 979 |
| | | ATGGA | | | AGCAT | |
| 2271 | RPOB_EC_3789_3812_F | TCAGTTCGGCGGTCAGCGC | 294 | RPOB_EC_3860_3890_R | TCGTCGGACTTAACGGTCAGCAT | 1107 |
| | | TTCGG | | | TTCCTGCA | |
| 2272 | RPOB_EC_3789_3812_F | TCAGTTCGGCGGTCAGCGC | 294 | RPOB_EC_3860_3890_2_R | TCGTCCGACTTAACGGTCAGCAT | 1102 |
| | | TTCGG | | | TTCCTGCA | |
| 2273 | RPOB_EC_3789_3812_F | TCAGTTCGGCGGTCAGCGC | 294 | RPOB_EC_3862_3890_R | TCGTCGGACTTAACGGTCAGCAT | 1106 |
| | | TTCGG | | | TTCCTG | |
| 2274 | RPOB_EC_3789_3812_F | TCAGTTCGGCGGTCAGCGC | 294 | RPOB_EC_3862_3890_2_R | TCGTCCGACTTAACGGTCAGCAT | 1101 |
| | | TTCGG | | | TTCCTG | |
| 2275 | RPOB_EC_3793_3812_F | TTCGGCGGTCAGCGCTTCGG | 674 | RPOB_EC_3865_3890_R | TCGTCGGACTTAACGGTCAGCAT | 1105 |
| | | | | | TTC | |
| 2276 | RPOB_EC_3793_3812_F | TTCGGCGGTCAGCGCTTCGG | 674 | RPOB_EC_3865_3890_2_R | TCGTCCGACTTAACGGTCAGCAT | 1100 |
| | | | | | TTC | |
| 2309 | MUPR_X75439_1658_1689_F | TCCTTTGATATATTATGCG | 352 | MUPR_X75439_1744_1773_R | TCCCTTCCTTAATATGAGAAGGA | 1030 |
| | | ATGGAAGGTTGGT | | | AACCACT | |
| 2310 | MUPR_X754390_1330_1353_F | TTCCTCCTTTTGAAAGCGA | 669 | MUPR_X75439_1413_1441_R | TGAGCTGGTGCTATATGAACAAT | 1171 |
| | | CGGTT | | | ACCAGT | |
| 2312 | MUPR_X75439_1314_1338_F | TTTCCTCCTTTTGAAAGCG | 704 | MUPR_X75439_1381_1409_R | TATATGAACAATACCAGTTCCTT | 931 |
| | | ACGGTT | | | CTGAGT | |
| 2313 | MUPR_X75439_2486_2516_F | TAATTGGGCTCTTTCTCGC | 172 | MUPR_X75439_2548_2574_R | TTAATCTGGCTGCGGAAGTGAAA | 1360 |
| | | TTAAACACCTTA | | | TCGT | |
| 2314 | MUPR_X75439_2547_2572_F | TACGATTTCACTTCCGCAG | 188 | MUPR_X75439_2605_2630_R | TCGTCCTCTCGAATCTCCGATAT | 1103 |
| | | CCAGATT | | | ACC | |
| 2315 | MUPR_X75439_2666_2696_F | TGCGTACAATACGCTTTAT | 513 | MUPR_X75439_2711_2740_R | TCAGATATAAATGGAACAAATGG | 981 |
| | | GAAATTTTAACA | | | AGCCACT | |
| 2316 | MUPR_X75439_2813_2843_F | TAATCAAGCATTGGAAGAT | 165 | MUPR_X75439_2867_2890_R | TCTGCATTTTTGCGAGCCTGTCTA | 1127 |
| | | GAAATGCATACC | | | | |
| 2317 | MUPR_X75439_884_914_F | TGACATGGACTCCCCCTAT | 447 | MUPR_X75439_977_1007_R | TGTACAATAAGGAGTCACCTTAT | 1317 |
| | | ATAACTCTTGAG | | | GTCCCTTA | |
| 2318 | CTXA_NC002505- | TGGTCTTATGCCAAGAGGA | 608 | CTXA_NC002505- | TCGTGCCTAACAAATCCCGTCTG | 1109 |
| | 1568114- | CAGAGTGAGT | | 1568114- | AGTTC | |
| | 1567341_114_142_F | | | 1567341_194_221_R | | |
| 2319 | CTXA_NC002505- | TCTTATGCCAAGAGGACAG | 411 | CTXA_NC002505- | TCGTGCCTAACAAATCCCGTCTG | 1109 |
| | 1568114- | AGTGAGTACT | | 1568114- | AGTTC | |
| | 1567341_117_145_F | | | 1567341_194_221_R | | |
| 2320 | CTXA_NC002505- | TGGTCTTATGCCAAGAGGA | 608 | CTXA_NC002505- | TAACAAATCCCGTCTGAGTTCCT | 855 |
| | 1568114- | CAGAGTGAGT | | 1568114- | CTTGCA | |
| | 1567341_114_142_F | | | 1567341_186_214_R | | |
| 2321 | CTXA_NC002505- | TCTTATGCCAAGAGGACAG | 411 | CTXA_NC002505- | TAACAAATCCCGTCTGAGTTCCT | 855 |
| | 1568114- | AGTGAGTACT | | 1568114- | CTTGCA | |
| | 1567341_117_145_F | | | 1567341_186_214_R | | |
| 2322 | CTXA_NC002505- | AGGACAGAGTGAGTACTTT | 27 | CTXA_NC002505- | TCCCGTCTGAGTTCCTCTTGCAT | 1027 |
| | 1568114- | GACCGAGGT | | 1568114- | GATCA | |
| | 1567341_129_156_F | | | 1567341_180_207_R | | |
| 2323 | CTXA_NC002505- | TGCCAAGAGGACAGAGTGA | 500 | CTXA_NC002505- | TAACAAATCCCGTCTGAGTTCCT | 855 |
| | 1568114- | GTACTTTGA | | 1568114- | CTTGCA | |
| | 1567341_122_149_F | | | 1567341_186_214_R | | |
| 2324 | INV_U22457-74- | TGCTTATTTACCTGCACTC | 530 | INV_U22457-74- | TGACCCAAAGCTGAAAGCTTTAC | 1154 |
| | 3772_831_858_F | CCACAACTG | | 3772_942_966_R | TG | |
| 2325 | INV_U22457-74- | TGAATGCTTATTTACCTGC | 438 | INV_U22457-74- | TAACTGACCCAAAGCTGAAAGCT | 864 |
| | 3772_827_857_F | ACTCCCACAACT | | 3772_942_970_R | TTACTG | |
| 2326 | INV_U22457-74- | TGCTGGTAACAGAGCCTTA | 526 | INV_U22457-74- | TGGGTTGCGTTGCAGATTATCTT | 1296 |
| | 3772_1555_1581_F | TAGGCGCA | | 3772_1619_1647_R | TACCAA | |
| 2327 | INV_U22457-74- | TGGTAACAGAGCCTTATAG | 598 | INV_U22457-74- | TCATAAGGGTTGCGTTGCAGATT | 987 |
| | 3772_1558_1585_F | GCGCATATG | | 3772_1622_1652_R | ATCTTTAC | |
| 2328 | ASD_NC006570- | TGAGGGTTTTATGCTTAAA | 459 | ASD_NC006570-439714- | TGATTCGATCATACGAGACATTA | 1188 |
| | 439714- | GTTGGTTTTATTGGTT | | 438608_54_84_R | AAACTGAG | |
| | 438608_3_37_F | | | | | |
| 2329 | ASD_NC006570- | TAAAGTTGGTTTTATTGGT | 149 | ASD_NC006570-439714- | TCAAAATCTTTTGATTCGATCAT | 948 |
| | 439714- | TGGCGCGGA | | 438608_66_95_R | ACGAGAC | |
| | 438608_18_45_F | | | | | |
| 2330 | ASD_NC006570- | TTAAAGTTGGTTTTATTGG | 647 | ASD_NC006570-439714- | TCCCAATCTTTTGATTCGATCAT | 1016 |
| | 439714- | TTGGCGCGGA | | 438608_67_95_R | ACGAGA | |
| | 438608_17_45_F | | | | | |
| 2331 | ASD_NC006570- | TTTTATGCTTAAAGTTGGT | 709 | ASD_NC006570-439714- | TCTGCCTGAGATGTCGAAAAAAA | 1128 |
| | 439714- | TTTATTGGTTGGC | | 438608_107_134_R | CGTTG | |
| | 438608_9_40_F | | | | | |
| 2332 | GALE_AF513299_171_200_F | TCAGCTAGACCTTTTAGGT | 280 | GALE_AF513299_241_271_R | TCTCACCTACAGCTTTAAAGCCA | 1122 |
| | | AAAGCTAAGCT | | | GCAAAATG | |
| 2333 | GALE_AF513299_168_199_F | TTATCAGCTAGACCTTTTA | 658 | GALE_AF513299_245_271_R | TCTCACCTACAGCTTTAAAGCCA | 1121 |
| | | GGTAAAGCTAAGC | | | GCAA | |
| 2334 | GALE_AF513299_168_199_F | TTATCAGCTAGACCTTTTA | 658 | GALE_AF513299_233_264_R | TACAGCTTTAAAGCCAGCAAAAT | 883 |
| | | GGTAAAGCTAAGC | | | GAATTACAG | |
| 2335 | GALE_AF513299_169_198_F | TCCCAGCTAGACCTTTTAG | 319 | GALE_AF513299_252_279_R | TTCAACACTCTCACCTACAGCTT | 1374 |
| | | GTAAAGCTAAG | | | TAAAG | |
| 2336 | PLA_AF053945_7371_7403_F | TTGAGAAGACATCCGGCTC | 680 | PLA_AF053945_7434_7468_R | TACGTATGTAAATTCCGCAAAGA | 900 |
| | | ACGTTATTATGGTA | | | CTTTGGCATTAG | |
| 2337 | PLA_AF053945_7377_7403_F | TGACATCCGGCTCACGTTA | 443 | PLA_AF053945_7428_7455_R | TCCGCAAAGACTTTGGCATTAGG | 1035 |
| | | TTATGGTA | | | TGTGA | |
| 2338 | PLA_AF053945_7377_7404_F | TGACATCCGGCTCACGTTA | 444 | PLA_AF053945_7430_7460_R | TAAATTCCGCAAAGACTTTGGCA | 854 |
| | | TTATGGTAC | | | TTAGGTGT | |
| 2339 | CAF_AF053947_33412_33441_F | TCCGTTATCGCCATTGCAT | 329 | CAF_AF053947_33498_33523_R | TAAGAGTGATGCGGGCTGGTTCA | 866 |
| | | TATTTGGAACT | | | ACA | |
| 2340 | CAF_AF053947_33426_33458_F | TGCATTATTTGGAACTATT | 499 | CAF_AF053947_33483_33507_R | TGGTTCAACAAGAGTTGCCGTTG | 1308 |
| | | GCAACTGCTAATGC | | | CA | |
| 2341 | CAF_AF053947_33407_33429_F | TCAGTTCCGTTATCGCCAT | 291 | CAF_AF053947_33483_33504_R | TTCAACAAGAGTTGCCGTTGCA | 1373 |
| | | TGCA | | | | |
| 2342 | CAF_AF053947_33407_33431_F | TCAGTTCCGTTATCGCCAT | 293 | CAF_AF053947_33494_33517_R | TGATGCGGGCTGGTTCAACAAGAG | 1184 |
| | | TGCATT | | | | |
| 2344 | GAPA_NC_002505_1_28_F_1 | TCAATGAACGATCAACAAG | 260 | GAPA_NC_002505_29_58_R_1 | TCCTTTATGCAACTTGGTATCAA | 1060 |
| | | TGATTGATG | | | CAGGAAT | |
| 2472 | OMPA_NC000117_68_89_F | TGCCTGTAGGGAATCCTGC | 507 | OMPA_NC000117_145_167_R | TCACACCAAGTAGTGCAAGGATC | 967 |
| | | TGA | | | | |
| 2473 | OMPA_NC000117_798_821_F | TGATTACCATGAGTGGCAA | 475 | OMPA_NC000117_865_893_R | TCAAAACTTGCTCTAGACCATTT | 947 |
| | | GCAAG | | | AACTCC | |
| 2474 | OMPA_NC000117_645_671_F | TGCTCAATCTAAACCTAAA | 521 | OMPA_NC000117_757_777_R | TGTCGCAGCATCTGTTCCTGC | 1328 |
| | | GTCGAAGA | | | | |
| 2475 | OMPA_NC000117_947_973_F | TAACTGCATGGAACCCTTC | 157 | OMPA_NC000117_1011_1040_R | TGACAGGACACAATCTGCATGAA | 1153 |
| | | TTTACTAG | | | GTCTGAG | |
| 2476 | OMPA_NC000117_774_795_F | TACTGGAACAAAGTCTGCG | 196 | OMPA_NC000117_871_894_R | TTCAAAAGTTGCTCGAGACCATTG | 1371 |
| | | ACC | | | | |
| 2477 | OMPA_NC000117_457_483_F | TTCTATCTCGTTGGTTTAT | 676 | OMPA_NC000117_511_534_R | TAAAGAGACGTTTGGTAGTTCAT | 851 |
| | | TCGGAGTT | | | TTGC | |
| 2478 | OMPA_NC000117_687_710_F | TAGCCCAGCACAATTTGTG | 212 | OMPA_NC000117_787_816_R | TTGCCATTCATGGTATTTAAGTG | 1406 |
| | | ATTCA | | | TAGCAGA | |
| 2479 | OMPA_NC000117_540_566_F | TGGCGTAGTAGAGCTATTT | 571 | OMPA_NC000117_649_672_R | TTCTTGAACGCGAGGTTTCGATTG | 1395 |
| | | ACAGACAC | | | | |
| 2480 | OMPA_NC000117_338_360_F | TGCACGATGCGGAATGGTT | 492 | OMPA_NC000117_417_444_R | TCCTTTAAAATAACCGCTAGTAG | 1058 |
| | | CACA | | | CTCCT | |
| 2481 | OMP2_NC000117_18_40_F | TATGACCAAACTCATCAGA | 234 | OMP2_NC000117_71_91_R | TCCCGCTGGCAAATAAACTCG | 1025 |
| | | CGAG | | | | |
| 2482 | OMP2_NC000117_354_382_F | TGCTACGGTAGGATCTCCT | 516 | OMP2_NC000117_445_471_R | TGGATCACTGCTTACGAACTCAG | 1270 |
| | | TATCCTATTG | | | CTTC | |
| 2483 | OMP2_NC000117_1297_1319_F | TGGAAAGGTGTTGCAGCTA | 537 | OMP2_NC000117_1396_1419_R | TACGTTTGTATCTTCTGCAGAACC | 903 |
| | | CTCA | | | | |
| 2484 | OMP2_NC000117_1465_1493_F | TCTGGTCCAACAAAAGGAA | 407 | OMP2_NC000117_1541_1569_R | TCCTTTCAATGTTACAGAAAACT | 1062 |
| | | CGATTACAGG | | | CTACAG | |
| 2485 | OMP2_NC000117_44_66_F | TGACGATCTTCGCGGTGAC | 450 | OMP2_NC000117_120_148_R | TGTCAGCTAAGCTAATAACGTTT | 1323 |
| | | TAGT | | | GTAGAG | |
| 2486 | OMP2_NC000117_166_190_F | TGACAGCGAAGAAGGTTAG | 441 | OMP2_NC000117_240_261_R | TTGACATCGTCCCTCTTCACAG | 1396 |
| | | ACTTGTCC | | | | |
| 2487 | GYRA_NC000117_514_536_F | TCAGGCATTGCGGTTGGGA | 287 | GYRA_NC000117_640_660_R | TGCTGTAGGGAAATCAGGGCC | 1251 |
| | | TGGC | | | | |
| 2488 | GYRA_NC000117_801_827_F | TGTGAATAAATCACGATTG | 636 | GYRA_NC000117_871_893_R | TTGTCAGACTCATCGCGAACATC | 1419 |
| | | ATTGAGCA | | | | |
| 2489 | GYRA_NC002952_219_242_F | TGTCATGGGTAAATATCAC | 632 | GYRA_NC002952_319_345_R | TCCATCCATAGAACCAAAGTTAC | 1010 |
| | | CCTCA | | | CTTG | |
| 2490 | GYRA_NC002952_964_983_F | TACAAGCACTCCCAGCTGCA | 176 | GYRA_NC002952_1024_1041_R | TCGCAGCGTGCGTGGCAC | 1073 |
| 2491 | GYRA_NC002952_1505_1520_F | TCGCCCGCGAGGACGT | 366 | GYRA_NC002952_1546_1562_R | TTGGTGCGCTTGGCGTA | 1416 |
| 2492 | GYRA_NC002952_59_81_F | TCAGCTACATCGACTATGC | 279 | GYRA_NC002952_124_143_R | TGGCGATGCACTGGCTTGAG | 1279 |
| | | GATG | | | | |
| 2493 | GYRA_NC002952_216_239_F | TGACGTCATCGGTAAGTAC | 452 | GYRA_NC002952_313_333_R | TCCGAAGTTGCCCTGGCCGTC | 1032 |
| | | CACCC | | | | |
| 2494 | GYRA_NC002952_219_242_2_F | TGTACTCGGTAAGTATCAC | 625 | GYRA_NC002952_308_330_R | TAAGTTACCTTGCCCGTCAACCA | 873 |
| | | CCGCA | | | | |
| 2495 | GYRA_NC002952_115_141_F | TGAGATGGATTTAAACCTG | 453 | GYRA_NC002952_220_242_R | TGCGGGTGATACTTACCGAGTAC | 1236 |
| | | TTCACCGC | | | | |
| 2496 | GYRA_NC002952_517_539_F | TCAGGCATTGCGGTTGGGA | 287 | GYRA_NC002952_643_663_R | TGCTGTAGGGAAATCAGGGCC | 1251 |
| | | TGGC | | | | |
| 2497 | GYRA_NC002952_273_293_F | TCGTATGGCTCAATGGTGG | 380 | GYRA_NC002952_338_360_R | TGCGGCAGCACTATCACCATCCA | 1234 |
| | | AG | | | | |
| 2498 | GYRA_NC000912_257_278_F | TGAGTAAGTTCCACCCGCA | 462 | GYRA_NC000912_346_370_R | TCGAGCCGAAGTTACCCTGTCCG | 1067 |
| | | CGG | | | TC | |
| 2504 | ARCC_NC003923- | TAGTpGATpAGAACpTpGT | 229 | ARCC_NC003923- | TCpTpTpTpCpGTATAAAAAGGA | 1116 |
| | 2725050- | AGGCpACpAATpCpGT | | 2725050- | CpCpAATpTpGG | |
| | 2724595_135_161P_F | | | 2724595_214_239P_R | | |
| 2505 | PTA_NC003923- | TCTTGTpTpTpATGCpTpG | 417 | PTA_NC003923-628885- | TACpACpCpTGGTpTpTpCpGTp | 904 |
| | 628885- | GTAAAGCAGATGG | | 629355_314_342P_R | TpTpTpGATGATpTpTpGTA | |
| | 629355_237_263P_F | | | | | |
| 2517 | CJMLST_ST1_1852_1883_F | TTTGCGGATGAAGTAGGTG | 708 | CJMLST_ST1_1945_1977_R | TGTTTTATGTGTAGTTGAGCTTA | 1355 |
| | | CCTATCTTTTTGC | | | CTACATGAGC | |
| 2518 | CJMLST_ST1_2963_2992_F | TGAAATTGCTACAGGCCCT | 428 | CJMLST_ST1_3073_3097_R | TCCCCATCTCCGCAAAGACAATA | 1020 |
| | | TTAGGACAAGG | | | AA | |
| 2519 | CJMLST_ST1_2350_2378_F | TGCTTTTGATGGTGATGCA | 535 | CJMLST_ST1_2447_2481_R | TCTACAACACTTGATTGTAATTT | 1117 |
| | | GATCGTTTGG | | | GCCTTGTTCTTT | |
| 2520 | CJMLST_ST1_654_684_F | TATGTCCAAGAAGCATAGC | 240 | CJMLST_ST1_725_756_R | TCGGAAACAAAGAATTCATTTTC | 1084 |
| | | AAAAAAAGCAAT | | | TGGTCCAAA | |
| 2521 | CJMLST_ST1_360_395_F | TCCTGTTATTCCTGAAGTA | 347 | CJMLST_ST1_454_487_R | TGCTATATGCTACAACTGGTTCA | 1245 |
| | | GTTAATCAAGTTTGTTA | | | AAAACATTAAG | |
| 2522 | CJMLST_ST1_1231_1258_F | TGGCAGTTTTACAAGGTGC | 564 | CJMLST_ST1_1312_1340_R | TTTAGCTACTATTCTAGCTGCCA | 1427 |
| | | TGTTTCATC | | | TTTCCA | |
| 2523 | CJMLST_ST1_3543_3574_F | TGCTGTAGCTTATCGCGAA | 529 | CJMLST_ST1_3656_3685_R | TCAAAGAACCAGCACCTAATTCA | 950 |
| | | ATGTCTTTGATTT | | | TCATTTA | |
| 2524 | CJMLST_ST1_1_17_F | TAAAACTTTTGCCGTAATG | 145 | CJMLST_ST1_55_84_R | TGTTCCAATAGCAGTTCCGCCCA | 1348 |
| | | ATGGGTGAAGATAT | | | AATTGAT | |
| 2525 | CJMLST_ST1_1312_1342_F | TGGAAATGGCAGCTAGAAT | 538 | CJMLST_ST1_1383_1417_R | TTTCCCCGATCTAAATTTGGATA | 1432 |
| | | AGTAGCTAAAAT | | | AGCCATAGGAAA | |
| 2526 | CJMLST_ST1_2254_2286_F | TGGGCCTAATGGGCTTAAT | 582 | CJMLST_ST1_2352_2379_R | TCCAAACGATCTGCATCACCATC | 996 |
| | | ATCAATGAAAATTG | | | AAAAG | |
| 2527 | CJMLST_ST1_1380_1411_F | TGCTTTCCTATGGCTTATC | 534 | CJMLST_ST1_1486_1520_R | TGCATGAAGCATAAAAACTGTAT | 1205 |
| | | CAAATTTAGATCG | | | CAAGTGCTTTTA | |
| 2528 | CJMLST_ST1_3413_3437_F | TTGTAAATGCCGGTGCTTC | 692 | CJMLST_ST1_3511_3542_R | TGCTTGCTCAAATCATCATAAAC | 1257 |
| | | AGATCC | | | AATTAAAGC | |
| 2529 | CJMLST_ST1_1130_1156_F | TACGCGTCTTGAAGCGTTT | 189 | CJMLST_ST1_1203_1230_R | TAGGATGAGCATTATCAGGGAAA | 920 |
| | | CGTTATGA | | | GAATC | |
| 2530 | CJMLST_ST1_2840_2872_F | TGGGGCTTTGCTTTATAGT | 591 | CJMLST_ST1_2940_2973_R | TAGCGATTTCTACTCCTAGAGTT | 917 |
| | | TTTTTACATTTAAG | | | GAAATTTCAGG | |
| 2531 | CJMLST_ST1_2058_2084_F | TATTCAAGGTGGTCCTTTG | 241 | CJMLST_ST1_2131_2162_R | TTGGTTCTTACTTGTTTTGCATA | 1417 |
| | | ATGCATGT | | | AACTTTCCA | |
| 2532 | CJMLST_ST1_553_585_F | TCCTGATGCTCAAAGTGCT | 344 | CJMLST_ST1_655_685_R | TATTGCTTTTTTTGCTATGCTTC | 942 |
| | | TTTTTAGATCCTTT | | | TTGGACAT | |
| 2564 | GLTA_NC002163- | TCATGTTGAGCTTAAACCT | 299 | GLTA_NC002163- | TTTTGCTCATGATCTGCATGAAG | 1443 |
| | 1604930- | ATAGAAGTAAAAGC | | 1604930- | CATAAA | |
| | 1604529_306_338_F | | | 1604529_352_380_R | | |
| 2565 | UNCA_NC002163- | TCCCCCACGCTTTAATTGT | 322 | UNCA_NC002163- | TCGACCTGGAGGACGACGTAAAA | 1065 |
| | 112166- | TTATGATGATTTGAG | | 112166- | TCA | |
| | 112647_80_113_F | | | 112647_146_171_R | | |
| 2566 | UNCA_NC002163- | TAATGATGAATTAGGTGCG | 170 | UNCA_NC002163- | TGGGATAACATTGGTTGGAATAT | 1285 |
| | 112166- | GGTTCTTT | | 112166- | AAGCAGAAACATC | |
| | 112647_233_259_F | | | 112647_294_329_R | | |
| 2567 | PGM_NC002163- | TCTTGATACTTGTAATGTG | 414 | PGM_NC002163-327773- | TCCATCGCCAGTTTTTGCATAAT | 1012 |
| | 327773- | GGCGATAAATATGT | | 328270_365_396_R | CGCTAAAAA | |
| | 328270_273_305_F | | | | | |
| 2568 | TKT_NC002163- | TTATGAAGCGTGTTCTTTA | 661 | TKT_NC002163- | TCAAAACGCATTTTTACATCTTC | 946 |
| | 1569415- | GCAGGACTTCA | | 1569415- | GTTAAAGGCTA | |
| | 1569873_255_284_F | | | 1569873_350_383_R | | |
| 2570 | GLTA_NC002163- | TCGTCTTTTTGATTCTTTC | 381 | GLTA_NC002163- | TGTTCATGTTTAAATGATCAGGA | 1347 |
| | 1604930- | CCTGATAATGC | | 1604930- | TAAAAAGCACT | |
| | 1604529_39_68_F | | | 1604529_109_142_R | | |
| 2571 | TKT_NC002163- | TGATCTTAAAAATTTCCGC | 472 | TKT_NC002163- | TGCCATAGCAAAGCCTACAGCATT | 1214 |
| | 1569415- | CAACTTCATTC | | 1569415- | | |
| | 1569903_33_62_F | | | 1569903_139_162_R | | |
| 2572 | TKT_NC002163- | TAAGGTTTATTGTCTTTGT | 164 | TKT_NC002163- | TACATCTCCTTCGATAGAAATTT | 886 |
| | 1569415- | GGAGATGGGGATTT | | 1569415- | CATTGCTATC | |
| | 1569903_207_239_F | | | 1569903_313_345_R | | |
| 2573 | TKT_NC002163- | TAGCCTTTAACGAAAATGT | 213 | TKT_NC002163- | TAAGACAAGGTTTTGTGGATTTT | 865 |
| | 1569415- | AAAAATGCGTTTTGA | | 1569415- | TTAGCTTGTT | |
| | 1569903_350_383_F | | | 1569903_449_481_R | | |
| 2574 | TKT_NC002163- | TTCAAAAACTCCAGGCCAT | 665 | TKT_NC002163- | TTGCCATAGCAAAGCCTACAGCA | 1405 |
| | 1569415- | CCTGAAATTTCAAC | | 1569415- | TT | |
| | 1569903_60_92_F | | | 1569903_139_163_R | | |
| 2575 | GLTA_NC002163- | TCGTCTTTTTGATTCTTTC | 382 | GLTA_NC002163- | TGCCATTTCCATGTACTCTTCTC | 1216 |
| | 1604930- | CCTGATAATGCTC | | 1604930- | TAACATT | |
| | 1604529_39_70_F | | | 1604529_139_168_R | | |
| 2576 | GLYA_NC002163- | TCAGCTATTTTTCCAGGTA | 281 | GLYA_NC002163- | ATTGCTTCTTACTTGCTTAGCAT | 756 |
| | 367572- | TCCAAGGTGG | | 367572- | AAATTTTCCA | |
| | 368079_386_414_F | | | 368079_476_508_R | | |
| 2577 | GLYA_NC002163- | TGGTGCGAGTGCTTATGCT | 611 | GLYA_NC002163- | TGCTCACCTGCTACAACAAGTCC | 1246 |
| | 367572- | CGTATTAT | | 367572- | AGCAAT | |
| | 368079_148_174_F | | | 368079_242_270_R | | |
| 2578 | GLYA_NC002163- | TGTAAGCTCTACAACCCAC | 622 | GLYA_NC002163- | TTCCACCTTGGATACCTGGAAAA | 1381 |
| | 367572- | AAAACCTTACG | | 367572- | ATAGCTGAAT | |
| | 368079_298_327_F | | | 368079_384_416_R | | |
| 2579 | GLYA_NC002163- | TGGTGGACATTTAACACAT | 614 | GLYA_NC002163- | TCAAGCTCTACACCATAAAAAAA | 961 |
| | 367572- | GGTGCAAA | | 367572- | GCTCTCA | |
| | 368079_1_27_F | | | 368079_52_81_R | | |
| 2580 | PGM_NC002163- | TGAGCAATGGGGCTTTGAA | 455 | PGM_NC002163-327746- | TTTGCTCTCCGCCAAAGTTTCCAC | 1438 |
| | 327746- | AGAATTTTTAAAT | | 328270_356_379_R | | |
| | 328270_254_285_F | | | | | |
| 2581 | PGM_NC002163- | TGAAAAGGGTGAAGTAGCA | 425 | PGM_NC002163-327746- | TGCCCCATTGCTCATGATAGTAG | 1219 |
| | 327746- | AATGGAGATAG | | 328270_241_267_R | CTAC | |
| | 328270_153_182_F | | | | | |
| 2582 | PGM_NC002163- | TGGCCTAATGGGCTTAATA | 568 | PGM_NC002163-327746- | TGCACGCAAACGCTTTACTTCAGC | 1200 |
| | 327746- | TCAATGAAAATTG | | 328270_79_102_R | | |
| | 328270_19_50_F | | | | | |
| 2583 | UNCA_NC002163- | TAAGCATGCTGTGGCTTAT | 160 | UNCA_NC002163- | TGCCCTTTCTAAAAGTCTTGAGT | 1220 |
| | 112166- | CGTGAAATG | | 112166- | GAAGATA | |
| | 112647_114_141_F | | | 112647_196_225_R | | |
| 2584 | UNCA_NC002163- | TGCTTCGGATCCAGCAGCA | 532 | UNCA_NC002163- | TGCATGCTTACTCAAATCATCAT | 1206 |
| | 112166- | CTTCAATA | | 112166- | AAACAATTAAAGC | |
| | 112647_3_29_F | | | 112647_88_123_R | | |
| 2585 | ASPA_NC002163- | TTAATTTGCCAAAAATGCA | 652 | ASPA_NC002163-96692- | TGCAAAAGTAACGGTTACATCTG | 1192 |
| | 96692- | ACCAGGTAG | | 97166_403_432_R | CTCCAAT | |
| | 97166_308_335_F | | | | | |
| 2586 | ASPA_NC002163- | TCGCGTTGCAACAAAACTT | 370 | ASPA_NC002163-96692- | TCATGATAGAACTACCTGGTTGC | 991 |
| | 96692- | TCTAAAGTATGT | | 97166_316_346_R | ATTTTTGG | |
| | 97166_228_258_F | | | | | |
| 2587 | GLNA_NC002163- | TGGAATGATGATAAAGATT | 547 | GLNA_NC002163- | TGAGTTTGAACCATTTCAGAGCG | 1176 |
| | 658085- | TCGCAGATAGCTA | | 658085- | AATATCTAC | |
| | 657609_244_275_F | | | 657609_340_371_R | | |
| 2588 | TKT_NC002163- | TCGCTACAGGCCCTTTAGG | 371 | TKT_NC002163- | TCCCCATCTCCGCAAAGACAATA | 1020 |
| | 1569415- | ACAAG | | 1569415- | AA | |
| | 1569903_107_130_F | | | 1569903_212_236_R | | |
| 2589 | TKT_NC002163- | TGTTCTTTAGCAGGACTTC | 642 | TKT_NC002163- | TCCTTGTGCTTCAAAACGCATTT | 1057 |
| | 1569415- | ACAAACTTGATAA | | 1569415- | TTACATTTTC | |
| | 1569903_265_296_F | | | 1569903_361_393_R | | |
| 2590 | GLYA_NC002163- | TGCCTATCTTTTTGCTGAT | 505 | GLYA_NC002163- | TCCTCTTGGGCCACGCAAAGTTTT | 1047 |
| | 367572- | ATAGCACATATTGC | | 367572- | | |
| | 368095_214_246_F | | | 368095_317_340_R | | |
| 2591 | GLYA_NC002163- | TCCTTTGATGCATGTAATT | 353 | GLYA_NC002163- | TCTTGAGCATTGGTTCTTACTTG | 1141 |
| | 367572- | GCTGCAAAAGC | | 367572- | TTTTGCATA | |
| | 368095_415_444_F | | | 368095_485_516_R | | |
| 2592 | PGM_NC002163_21_54_F | TCCTAATGGACTTAATATC | 332 | PGM_NC002163_116_142_R | TCAAACGATCCGCATCACCATCA | 949 |
| | | AATGAAAATTGTGGA | | | AAAG | |
| 2593 | PGM_NC002163_149_176_F | TAGATGAAAAAGGCGAAGT | 207 | PGM_NC002163_247_277_R | TCCCCTTTAAAGCACCATTACTC | 1023 |
| | | GGCTAATGG | | | ATTATAGT | |
| 2594 | GLNA_NC002163- | TGTCCAAGAAGCATAGCAA | 633 | GLNA_NC002163- | TCAAAAACAAAGAATTCATTTTC | 945 |
| | 658085- | AAAAAGCAA | | 658085- | TGGTCCAAA | |
| | 657609_79_106_F | | | 657609_148_179_R | | |
| 2595 | ASPA_NC002163- | TCCTGTTATTCCTGAAGTA | 347 | ASPA_NC002163-96685- | TCAAGCTATATGCTACAACTGGT | 960 |
| | 96685- | GTTAATCAAGTTTGTTA | | 97196_467_497_R | TCAAAAAC | |
| | 97196_367_402_F | | | | | |
| 2596 | ASPA_NC002163- | TGCCGTAATGATAGGTGAA | 502 | ASPA_NC002163-96685- | TACAACCTTCGGATAATCAGGAT | 880 |
| | 96685- | GATATACAAAGAGT | | 97196_95_127_R | GAGAATTAAT | |
| | 97196_1_33_F | | | | | |
| 2597 | ASPA_NC002163- | TGGAACAGGAATTAATTCT | 540 | ASPA_NC002163-96685- | TAAGCTCCCGTATCTTGAGTCGC | 872 |
| | 96685- | CATCCTGATTATCC | | 97196_185_210_R | CTC | |
| | 97196_85_117_F | | | | | |
| 2598 | PGM_NC002163- | TGGCAGCTAGAATAGTAGC | 563 | PGM_NC002163-327746- | TCACGATCTAAATTTGGATAAGC | 975 |
| | 327746- | TAAAATCCCTAC | | 328270_230_261_R | CATAGGAAA | |
| | 328270_165_195_F | | | | | |
| 2599 | PGM_NC002163- | TGGGTCGTGGTTTTACAGA | 593 | PGM_NC002163-327746- | TTTTGCTCATGATCTGCATGAAG | 1443 |
| | 327746- | AAATTTCTTATATATG | | 328270_353_381_R | CATAAA | |
| | 328270_252_286_F | | | | | |
| 2600 | PGM_NC002163- | TGGGATGAAAAAGCGTTCT | 577 | PGM_NC002163-327746- | TGATAAAAAGCACTAAGCGATGA | 1178 |
| | 327746- | TTTATCCATGA | | 328270_95_123_R | AACAGC | |
| | 328270_1_30_F | | | | | |
| 2601 | PGM_NC002163- | TAAACACGGCTTTCCTATG | 146 | PGM_NC002163-327746- | TCAAGTGCTTTTACTTCTATAGG | 963 |
| | 327746- | GCTTATCCAAAT | | 328270_314_345_R | TTTAAGCTC | |
| | 328270_220_250_F | | | | | |
| 2602 | UNCA_NC002163- | TGTAGCTTATCGCGAAATG | 628 | UNCA_NC002163- | TGCTTGCTCTTTCAAGCAGTCTT | 1258 |
| | 112166- | TCTTTGATTTT | | 112166- | GAATGAAG | |
| | 112647_123_152_F | | | 112647_199_229_R | | |
| 2603 | UNCA_NC002163- | TCCAGATGGACAAATTTTC | 313 | UNCA_NC002163- | TCCGAAACTTGTTTTGTAGCTTT | 1031 |
| | 112166- | TTAGAAACTGATTT | | 112166- | AATTTGAGC | |
| | 112647_333_365_F | | | 112647_430_461_R | | |
| 2734 | GYRA_AY291534_237_264_F | TCACCCTCATGGTGATTCA | 265 | GYRA_AY291534_268_288_R | TTGCGCCATACGTACCATCGT | 1407 |
| | | GCTGTTTAT | | | | |
| 2735 | GYRA_AY291534_224_252_F | TAATCGGTAAGTATCACCC | 167 | GYRA_AY291534_256_285_R | TGCCATACGTACCATCGTTTCAT | 1213 |
| | | TCATGGTGAT | | | AAACAGC | |
| 2736 | GYRA_AY291534_170_198_F | TAGGAATTACGGCTGATAA | 221 | GYRA_AY291534_268_288_R | TTGCGCCATACGTACCATCGT | 1407 |
| | | AGCGTATAAA | | | | |
| 2737 | GYRA_AY291534_224_252_F | TAATCGGTAAGTATCACCC | 167 | GYRA_AY291534_319_346_R | TATCGACAGATCCAAAGTTACCA | 935 |
| | | TCATGGTGAT | | | TGCCC | |
| 2738 | GYRA_NC002953- | TAAGGTATGACACCGGATA | 163 | GYRA_NC002953-7005- | TCTTGAGCCATACGTACCATTGC | 1142 |
| | 7005- | AATCATATAAA | | 9668_265_287_R | | |
| | 9668_166_195_F | | | | | |
| 2739 | GYRA_NC002953- | TAATGGGTAAATATCACCC | 171 | GYRA_NC002953-7005- | TATCCATTGAACCAAAGTTACCT | 933 |
| | 7005- | TCATGGTGAC | | 9668_316_343_R | TGGCC | |
| | 9668_221_249_F | | | | | |
| 2740 | GYRA_NC002953- | TAATGGGTAAATATCACCC | 171 | GYRA_NC002953-7005- | TAGCCATACGTACCATTGCTTCA | 912 |
| | 7005- | TCATGGTGAC | | 9668_253_283_R | TAAATAGA | |
| | 9668_221_249_F | | | | | |
| 2741 | GYRA_NC002953- | TCACCCTCATGGTGACTCA | 264 | GYRA_NC002953-7005- | TCTTGAGCCATACGTACCATTGC | 1142 |
| | 7005- | TCTATTTAT | | 9668_265_287_R | | |
| | 9668_234_261_F | | | | | |
| 2842 | CAPC_AF188935- | TGGGATTATTGTTATCCTG | 578 | CAPC_AF188935-56074- | TGGTAACCCTTGTCTTTGAATTG | 1299 |
| | 56074- | TTATGCCATTTGAGA | | 55628_348_378_R | TATTTGCA | |
| | 55628_271_304_F | | | | | |
| 2843 | CAPC_AF188935- | TGATTATTGTTATCCTGTT | 476 | CAPC_AF188935-56074- | TGTAACCCTTGTCTTTGAATpTp | 1314 |
| | 56074- | ATGCpCpATpTpTpGAG | | 55628_349_377P_R | GTATpTpTpGC | |
| | 55628_273_303P_F | | | | | |
| 2844 | CAPC_AF188935- | TCCGTTGATTATTGTTATC | 331 | CAPC_AF188935-56074- | TGTTAATGGTAACCCTTGTCTTT | 1344 |
| | 56074- | CTGTTATGCCATTTGAG | | 55628_349_384_R | GAATTGTATTTGC | |
| | 55628_268_303_F | | | | | |
| 2845 | CAPC_AF188935- | TCCGTTGATTATTGTTATC | 331 | CAPC_AF188935-56074- | TAACCCTTGTCTTTGAATTGTAT | 860 |
| | 56074- | CTGTTATGCCATTTGAG | | 55628_337_375_R | TTGCAATTAATCCTGG | |
| | 55628_268_303_F | | | | | |
| 2846 | PARC_X95819_33_58_F | TCCAAAAAAATCAGCGCGT | 302 | PARC_X95819_121_153_R | TAAAGGATAGCGGTAACTAAATG | 852 |
| | | ACAGTGG | | | GCTGAGCCAT | |
| 2847 | PARC_X95819_65_92_F | TACTTGGTAAATACCACCC | 199 | PARC_X95819_157_178_R | TACCCCAGTTCCCCTGACCTTC | 889 |
| | | ACATGGTGA | | | | |
| 2848 | PARC_X95819_69_93_F | TGGTAAATACCACCCACAT | 596 | PARC_X95819_97 128_R | TGAGCCATGAGTACCATGGCTTC | 1169 |
| | | GGTGAC | | | ATAACATGC | |
| 2849 | PARC_NC003997- | TTCCGTAAGTCGGCTAAAA | 668 | PARC_NC003997- | TCCAAGTTTGACTTAAACGTACC | 1001 |
| | 3362578- | CAGTCG | | 3362578- | ATCGC | |
| | 3365001_181_205_F | | | 3365001_256_283_R | | |
| 2850 | PARC_NC003997- | TGTAACTATCACCCGCACG | 621 | PARC_NC003997- | TCGTCAACACTACCATTATTACC | 1099 |
| | 3362578- | GTGAT | | 3362578- | ATGCATCTC | |
| | 3365001_217_240_F | | | 3365001_304_335_R | | |
| 2851 | PARC_NC003997- | TGTAACTATCACCCGCACG | 621 | PARC_NC003997- | TGACTTAAACGTACCATCGCTTC | 1162 |
| | 3362578- | GTGAT | | 3362578- | ATATACAGA | |
| | 3365001_217_240_F | | | 3365001_244_275_R | | |
| 2852 | GYRA_AY642140_- | TAAATCTGCCCGTGTCGTT | 150 | GYRA_AY642140_71_100_R | TGCTAAAGTCTTGAGCCATACGA | 1242 |
| | 1_24_F | GGTGAC | | | ACAATGG | |
| 2853 | GYRA_AY642140_26_54_F | TAATCGGTAAATATCACCC | 166 | GYRA_AY642140_121_146_R | TCGATCGAACCGAAGTTACCCTG | 1069 |
| | | GCATGGTGAC | | | ACC | |
| 2854 | GYRA_AY642140_26_54_F | TAATCGGTAAATATCACCC | 166 | GYRA_AY642140_58_89_R | TGAGCCATACGAACAATGGTTTC | 1168 |
| | | GCATGGTGAC | | | ATAAACAGC | |
| 2860 | CYA_AF065404_1348_1379_F | TCCAACGAAGTACAATACA | 305 | CYA_AF065404_1448_1472_R | TCAGCTGTTAACGGCTTCAAGAC | 983 |
| | | AGACAAAAGAAGG | | | CC | |
| 2861 | LEF_BA_AF065404_751_781_F | TCGAAAGCTTTTGCATATT | 354 | LEF_BA_AF065404_843_881_R | TCTTTAAGTTCTTCCAAGGATAG | 1144 |
| | | ATATCGAGCCAC | | | ATTTATTTCTTGTTCG | |
| 2862 | LEF_BA_AF065404_762_788_F | TGCATATTATATCGAGCCA | 498 | LEF_BA_AF065404_843_881_R | TCTTTAAGTTCTTCCAAGGATAG | 1144 |
| | | CAGCATCG | | | ATTTATTTCTTGTTCG | |
| 2917 | MUTS_AY698802_106_125_F | TCCGCTGAATCTGTCGCCGC | 326 | MUTS_AY698802_172_193_R | TGCGGTCTGGCGCATATAGGTA | 1237 |
| 2918 | MUTS_AY698802_172_192_F | TACCTATATGCGCCAGACC | 187 | MUTS_AY698802_228_252_R | TCAATCTCGACTTTTTGTGCCGG | 965 |
| | | GC | | | TA | |
| 2919 | MUTS_AY698802_228_252_F | TACCGGCGCAAAAAGTCGA | 186 | MUTS_AY698802_314_342_R | TCGGTTTCAGTCATCTCCACCAT | 1097 |
| | | GATTGG | | | AAAGGT | |
| 2920 | MUTS_AY698802_315_342_F | TCTTTATGGTGGAGATGAC | 419 | MUTS_AY698802_413_433_R | TGCCAGCGACAGACCATCGTA | 1210 |
| | | TGAAACCGA | | | | |
| 2921 | MUTS_AY698802_394_411_F | TGGGCGTGGAACGTCCAC | 585 | MUTS_AY698802_497_519_R | TCCGGTAACTGGGTCAGCTCGAA | 1040 |
| 2922 | AB_MLST-11- | TGGGcGATGCTGCgAAATG | 583 | AB_MLST-11- | TAGTATCACCACGTACACCCGGA | 923 |
| | OIF007_991_1018_F | GTTAAAAGA | | OIF007_1110_1137_R | TCAGT | |
| 2927 | GAPA_NC002505_694_721_F | TCAATGAACGACCAACAAG | 259 | GAPA_NC_002505_29_58_R_1 | TCCTTTATGCAACTTGGTATCAA | 1060 |
| | | TGATTGATG | | | CAGGAAT | |
| 2928 | GAPA_NC002505_694_721_2_F | TCGATGAACGACCAACAAG | 361 | GAPA_NC002505_769_798_3_R | TCCTTTATGCAACTTGGTATCAA | 1061 |
| | | TGATTGATG | | | CCGGAAT | |
| 2929 | GAPA_NC002505_694_721_2_F | TCGATGAACGACCAACAAG | 361 | GAPA_NC002505_769_798_3_R | TCCTTTATGCAACTTAGTATCAA | 1059 |
| | | TGATTGATG | | | CCGGAAT | |
| 2932 | INFB_EC_1364_1394_F | TTGCTCGTGGTGCACAAGT | 688 | INFB_EC_1439_1468_R | TTGCTGCTTTCGCATGGTTAATC | 1410 |
| | | AACGGATATTAC | | | GCTTCAA | |
| 2933 | INFB_EC_1364_1394_2_F | TTGCTCGTGGTGCAIAAGT | 689 | INFB_EC_1439_1468_R | TGCTGCTTTCGCATGGTTAATC | 1410 |
| | | AACGGATATIAC | | | GCTTCAA | |
| 2934 | INFB_EC_80_110_F | TTGCCCGCGGTGCGGAAGT | 685 | INFB_EC_1439_1468_R | TTGCTGCTTTCGCATGGTTAATC | 1410 |
| | | AACCGATATTAC | | | GCTTCAA | |
| 2949 | ACS_NC002516- | TCGGCGCCTGCCTGATGA | 376 | ACS_NC002516-970624- | TGGACCACGCCGAAGAACGG | 1265 |
| | 970624- | | | 971013_364_383_R | | |
| | 971013_299_316_F | | | | | |
| 2950 | ARO_NC002516- | TCACCGTGCCGTTCAAGGA | 267 | ARO_NC002516-26883- | TGTGTTGTCGCCGCGCAG | 1341 |
| | 26883- | AGAG | | 27380_111_128_R | | |
| | 27380_4_26_F | | | | | |
| 2951 | ARO_NC002516- | TTTCGAAGGGCCTTTCGAC | 705 | ARO_NC002516-26883- | TCCTTGGCATACATCATGTCGTA | 1056 |
| | 26883- | CTG | | 27380_459_484_R | GCA | |
| | 27380_356_377_F | | | | | |
| 2952 | GUA_NC002516- | TGGACTCCTCGGTGGTCGC | 551 | GUA_NC002516- | TCGGCGAACATGGCCATCAC | 1091 |
| | 4226546- | | | 4226546- | | |
| | 4226174_23_41_F | | | 4226174_127_146_R | | |
| 2953 | GUA_NC002516- | TGACCAGGTGATGGCCATG | 448 | GUA_NC002516- | TGCTTCTCTTCCGGGTCGGC | 1256 |
| | 4226546- | TTCG | | 4226546- | | |
| | 4226174_120_142_F | | | 4226174_214_233_R | | |
| 2954 | GUA_NC002516- | TTTTGAAGGTGATCCGTGC | 710 | GUA_NC002516- | TGCTTGGTGGCTTCTTCGTCGAA | 1259 |
| | 4226546- | CAACG | | 4226546- | | |
| | 4226174_155_178_F | | | 4226174_265_287_R | | |
| 2955 | GUA_NC002516- | TTCCTCGGCCGCCTGGC | 670 | GUA_NC002516- | TGCGAGGAACTTCACGTCCTGC | 1229 |
| | 4226546- | | | 4226546- | | |
| | 4226174_190_206_F | | | 4226174_288_309_R | | |
| 2956 | GUA_NC002516- | TCGGCCGCACCTTCATCGA | 374 | GUA_NC002516- | TCGTGGGCCTTGCCGGT | 1111 |
| | 4226546- | AGT | | 4226546- | | |
| | 4226174_242_263_F | | | 4226174_355_371_R | | |
| 2957 | MUT_NC002516- | TGGAAGTCATCAAGCGCCT | 545 | MUT_NC002516- | TCACGGGCCAGCTCGTCT | 978 |
| | 5551158- | GGC | | 5551158- | | |
| | 5550717_5_26_F | | | 5550717_99_116_R | | |
| 2958 | MUT_NC002516- | TCGAGCAGGCGCTGCCG | 358 | MUT_NC002516- | TCACCATGCGCCCGTTCACATA | 971 |
| | 5551158- | | | 5551158- | | |
| | 5550717_152_168_F | | | 5550717_256_277_R | | |
| 2959 | NUO_NC002516- | TCAACCTCGGCCCGAACCA | 249 | NUO_NC002516- | TCGGTGGTGGTAGCCGATCTC | 1095 |
| | 2984589- | | | 2984589- | | |
| | 2984954_8_26_F | | | 2984954_97_117_R | | |
| 2960 | NUO_NC002516- | TACTCTCGGTGGAGAAGCT | 195 | NUO_NC002516- | TTCAGGTACAGCAGGTGGTTCAG | 1376 |
| | 2984589- | CGC | | 2984589- | GAT | |
| | 2984954_218_239_F | | | 2984954_301_326_R | | |
| 2961 | PPS_NC002516- | TCCACGGTCATGGAGCGCTA | 311 | PPS_NC002516- | TCCATTTCCGACACGTCGTTGAT | 1014 |
| | 1915014- | | | 1915014- | CAC | |
| | 1915383_44_63_F | | | 1915383_140_165_R | | |
| 2962 | PPS_NC002516- | TCGCCATCGTCACCAACCG | 365 | PPS_NC002516- | TCCTGGCCATCCTGCAGGAT | 1052 |
| | 1915014- | | | 1915014- | | |
| | 1915383_240_258_F | | | 1915383_341_36_R | | |
| 2963 | TRP_NC002516- | TGCTGGTACGGGTCGAGGA | 527 | TRP_NC002516-671831- | TCGATCTCCTTGGCGTCCGA | 1071 |
| | 671831- | | | 672273_131_150_R | | |
| | 672273_24_42_F | | | | | |
| 2964 | TRP_NC002516- | TGCACATCGTGTCCAACGT | 490 | TRP_NC002516-671831- | TGATCTCCATGGCGCGGATCTT | 1182 |
| | 671831- | CAC | | 672273_362_383_R | | |
| | 672273_261_282_F | | | | | |
| 2972 | AB_MLST-11- | TGGGIGATGCTGCIAAATG | 592 | AB_MLST-11- | TAGTATCACCACGTACICCIGGA | 924 |
| | OIF007_1007_1034_F | GTTAAAAGA | | OIF007_1126_1153_R | TCAGT | |
| 2993 | OMPU_NC002505- | TTCCCACCGATATCATGGC | 667 | OMPU_NC002505_544_567_R | TCGGTCAGCAAAACGGTAGCTTGC | 1094 |
| | 674828- | TTACCACGG | | | | |
| | 675880_428_455_F | | | | | |
| 2994 | GAPA_NC002505- | TCCTCAATGAACGAICAAC | 335 | GAPA_NC002505- | TTTTCCCTTTATGCAACTTAGTA | 1442 |
| | 506780- | AAGTGATTGATG | | 506780- | TCAACIGGAAT | |
| | 507937_691_721_F | | | 507937_769_802_R | | |
| 2995 | GAPA_NC002505- | TCCTCIATGAACGAICAAC | 339 | GAPA_NC002505- | TCCATACCTTTATGCAACTTIGT | 1008 |
| | 506780- | AAGTGATTGATG | | 506780- | ATCAACIGGAAT | |
| | 507937_691_721_2_F | | | 507937_769_803_R | | |
| 2996 | GAPA_NC002505- | TCTCGATGAACGACCAACA | 396 | GAPA_NC002505- | TCGGAAATATTCTTTCAATACCT | 1085 |
| | 506780- | AGTGATTGATG | | 506780- | TTATGCAACT | |
| | 507937_692_721_F | | | 507937_785_817_R | | |
| 2997 | GAPA_NC002505- | TCCTCGATGAACGAICAAC | 337 | GAPA_NC002505- | TCGGAAATATTCTTTCAATACCT | 1085 |
| | 506780- | AAGTIATTGATG | | 506780- | TTATGCAACT | |
| | 507937_691_721_3_F | | | 507937_785_817_R | | |
| 2998 | GAPA_NC002505- | TCCTCAATGAATGATCAAC | 336 | GAPA_NC002505- | TCGGAAATATTCTTTCAATICCT | 1087 |
| | 506780- | AAGTGATTGATG | | 506780- | TTITGCAACTT | |
| | 507937_691_721_4_F | | | 507937_784_817_R | | |
| 2999 | GAPA_NC002505- | TCCTCIATGAAIGAICAAC | 340 | GAPA_NC002505- | TCGGAAATATTCTTTCAATACCT | 1086 |
| | 506780- | AAGTIATTGATG | | 506780- | TTATGCAACTT | |
| | 507937_691_721_5_F | | | 507937_784_817_2_R | | |
| 3000 | GAPA_NC002505- | TCCTCGATGAATGAICAAC | 338 | GAPA_NC002505- | TTTCAATACCTTTATGCAACTTI | 1430 |
| | 506780- | AAGTIATTGATG | | 506780- | GTATCAACIGGAAT | |
| | 507937_691_721_6_F | | | 507937_769_805_R | | |
| 3001 | CTXB_NC002505- | TCAGCATATGCACATGGAA | 275 | CTXB_NC002505- | TCCCGGCTAGAGATTCTGTATAC | 1026 |
| | 1566967- | CACCTCA | | 1566967- | GA | |
| | 1567341_46_71_F | | | 1567341_139_163_R | | |
| 3002 | CTXB_NC002505- | TCAGCATATGCACATGGAA | 274 | CTXB_NC002505- | TCCGGCTAGAGATTCTGTATACG | 1038 |
| | 1566967- | CACCTC | | 1566967- | AAAATATC | |
| | 1567341_46_70_F | | | 1567341_132_162_R | | |
| 3003 | CTXB_NC002505- | TCAGCATATGCACATGGAA | 274 | CTXB_NC002505- | TGCCGTATACGAAAATATCTTAT | 1225 |
| | 1566967- | CACCTC | | 1566967- | CATTTAGCGT | |
| | 1567341_46_70_F | | | 1567341_118_150_R | | |
| 3004 | TUFB_NC002758- | TACAGGCCGTGTTGAACGT | 180 | TUFB_NC002758- | TCAGCGTAGTCTAATAATTTACG | 982 |
| | 615038- | GG | | 615038- | GAACATTTC | |
| | 616222_684_704_F | | | 616222_778_809_R | | |
| 3005 | TUFB_NC002758- | TGCCGTGTTGAACGTGGTC | 503 | TUFB_NC002758- | TGCTTCAGCGTAGTCTAATAATT | 1255 |
| | 615038- | AAAT | | 615038- | TACGGAAC | |
| | 616222_688_710_F | | | 616222_783_813_R | | |
| 3006 | TUFB_NC002758- | TGTGGTCAAATCAAAGTTG | 638 | TUFB_NC002758- | TGCGTAGTCTAATAATTTACGGA | 1238 |
| | 615038- | GTGAAGAA | | 615038- | ACATTTC | |
| | 616222_700_726_F | | | 616222_778_807_R | | |
| 3007 | TUFB_NC002758- | TGGTCAAATCAAAGTTGGT | 607 | TUFB_NC002758- | TGCGTAGTCTAATAATTTACGGA | 1238 |
| | 615038- | GAAGAA | | 615038- | ACATTTC | |
| | 616222_702_726_F | | | 616222_778_807_R | | |
| 3008 | TUFB_NC002758- | TGAACGTGGTCAAATCAAA | 431 | TUFB_NC002758- | TCACCAGCTTCAGCGTAGTCTAA | 970 |
| | 615038- | GTTGGTGAAGAA | | 615038- | TAATTTACGGA | |
| | 616222_696_726_F | | | 616222_785_818_R | | |
| 3009 | TUFB_NC002758- | TCGTGTTGAACGTGGTCAA | 386 | TUFB_NC002758- | TCTTCAGCGTAGTCTAATAATTT | 1134 |
| | 615038- | ATCAAAGT | | 615038- | ACGGAACATTTC | |
| | 616222_690_716_F | | | 616222_778_812_R | | |
| 3010 | MECI- | TCACATATCGTGAGCAATG | 261 | MECI-R_NC003923- | TGTGATATGGAGGTGTAGAAGGTG | 1332 |
| | R_NC003923- | AACTG | | 41798-41609_89_112_R | | |
| | 41798- | | | | | |
| | 41609_36_59_F | | | | | |
| 3011 | MECI- | TGGGCGTGAGCAATGAACT | 584 | MECI-R_NC003923- | TGGGATGGAGGTGTAGAAGGTGT | 1287 |
| | R_NC003923- | GATTATAC | | 41798-41609_81_110_R | TATCATC | |
| | 41798- | | | | | |
| | 41609_40_66_F | | | | | |
| 3012 | MECI- | TGGACACATATCGTGAGCA | 549 | MECI-R_NC003923- | TGGGATGGAGGTGTAGAAGGTGT | 1286 |
| | R_NC003923- | ATGAACTGA | | 41798-41609_81_110_R | TATCATC | |
| | 41798- | | | | | |
| | 41609_33_60_2_F | | | | | |
| 3013 | MECI- | TGGGTTTACACATATCGTG | 595 | MECI-R_NC003923- | TGGGGATATGGAGGTGTAGAAGG | 1290 |
| | R_NC003923- | AGCAATGAACTGA | | 41798-41609_81_113_R | TGTTATCATC | |
| | 41798- | | | | | |
| | 41609_29_60_F | | | | | |
| 3014 | MUPR_X75439_2490_2514_F | TGGGCTCTTTCTCGCTTAA | 587 | MUPR_X75439_2548_2570_R | TCTGGCTGCGGAAGTGAAATCGT | 1130 |
| | | ACACCT | | | | |
| 3015 | MUPR_X75439_2490_2513_F | TGGGCTCTTTCTCGCTTAA | 586 | MUPR_X75439_2547_2568_R | TGGCTGCGGAAGTGAAATCGTA | 1281 |
| | | ACACC | | | | |
| 3016 | MUPR_X75439_2482_2510_F | TAGATAATTGGGCTCTTTC | 205 | MUPR_X75439_2551_2573_R | TAATCTGGCTGCGGAAGTGAAAT | 876 |
| | | TCGCTTAAAC | | | | |
| 3017 | MUPR_X75439_2490_2514_F | TGGGCTCTTTCTCGCTTAA | 587 | MUPR_X75439_2549_2573_R | TAATCTGGCTGCGGAAGTGAAAT | 877 |
| | | ACACCT | | | CG | |
| 3018 | MUPR_X75439_2482_2510_F | TAGATAATTGGGCTCTTTC | 205 | MUPR_X75439_2559_2589_R | TGGTATATTCGTTAATTAATCTG | 1303 |
| | | TCGCTTAAAC | | | GCTGCGGA | |
| 3019 | MUPR_X75439_2490_2514_F | TGGGCTCTTTCTCGCTTAA | 587 | MUPR_X75439_2554_2581_R | TCGTTAATTAATCTGGCTGCGGA | 1112 |
| | | ACACCT | | | AGTGA | |
| 3020 | AROE_NC003923- | TGATGGCAAGTGGATAGGG | 474 | AROE_NC003923- | TAAGCAATACCTTTACTTGCACC | 868 |
| | 1674726- | TATAATACAG | | 1674726- | ACCT | |
| | 1674277_204_232_F | | | 1674277_309_335_R | | |
| 3021 | AROE_NC003923- | TGGCGAGTGGATAGGGTAT | 570 | AROE_NC003923- | TTCATAAGCAATACCTTTACTTG | 1378 |
| | 1674726- | AATACAG | | 1674726- | CACCAC | |
| | 1674277_207_232_F | | | 1674277_311_339_R | | |
| 3022 | AROE_NC003923- | TGGCpAAGTpGGATpAGGG | 572 | AROE_NC003923- | TAAGCAATACCpTpTpTpACTpT | 867 |
| | 1674726- | TpATpAATpACpAG | | 1674726- | pGCpACpCpAC | |
| | 1674277_207_232P_F | | | 1674277_311_335P_R | | |
| 3023 | ARCC_NC003923- | TCTGAAATGAATAGTGATA | 398 | ARCC_NC003923- | TCTTCTTCTTTCGTATAAAAAGG | 1137 |
| | 2725050- | GAACTGTAGGCAC | | 2725050- | ACCAATTGG | |
| | 2724595_124_155_F | | | 2724595_214_245_R | | |
| 3024 | ARCC_NC003923- | TGAATAGTGATAGAACTGT | 437 | ARCC_NC003923- | TCTTCTTTCGTATAAAAAGGACC | 1139 |
| | 2725050- | AGGCACAATCGT | | 2725050- | AATTGGTT | |
| | 2724595_131_161_F | | | 2724595_212_242_R | | |
| 3025 | ARCC_NC003923- | TGAATAGTGATAGAACTGT | 437 | ARCC_NC003923- | TGCGCTAATTCTTCAACTTCTTC | 1232 |
| | 2725050- | AGGCACAATCGT | | 2725050- | TTTCGT | |
| | 2724595_131_161_F | | | 2724595_232_260_R | | |
| 3026 | PTA_NC003923- | TACAATGCTTGTTTATGCT | 177 | PTA_NC003923-628885- | TGTTCTTGATACACCTGGTTTCG | 1350 |
| | 628885- | GGTAAAGCAG | | 629355_322_351_R | TTTTGAT | |
| | 629355_231_259_F | | | | | |
| 3027 | PTA_NC003923- | TACAATGCTTGTTTATGCT | 177 | PTA_NC003923-628885- | TGGTACACCTGGTTTCGTTTTGA | 1301 |
| | 628885- | GGTAAAGCAG | | 629355_314_345_R | TGATTTGTA | |
| | 629355_231_259_F | | | | | |
| 3028 | PTA_NC003923- | TCTTGTTTATGCTGGTAAA | 418 | PTA_NC003923-628885- | TGTTCTTGATACACCTGGTTTCG | 1350 |
| | 628885- | GCAGATGG | | 629355_322_351_R | TTTTGAT | |
| | 629355_237_263_F | | | | | |
| 3106 | TSST1_NC002758.2- | TCGTCATCAGCTAACTCAA | 1465 | TSST1_NC002758.2- | TCACTTTGATATGTGGATCCGTC | 1466 |
| | 2137509- | ATACATGGA | | 2137509-2138213_593- | ATTCA | |
| | 2138213_519_546_F | | | 620_R | | |
| 3105 | TSST1_NC002758.2_35_57_F | TAAGCCCTTTGTTGCTTGC | 1467 | TSST1_NC002758.2_146_173_R | TCAGACCCACTACTATACCAGTC | 1468 |
| | | GACA | | | TAGCA | |
| 3107 | TSST1_NC002758.2_334_357_F | TGCCAACATACTAGCGAAG | 1469 | TSST1_NC002758.2_415_445_R | TCCCATGAACCTTAACTTTTAAA | 1470 |
| | | GAACT | | | GGTAGTTC | |

Primer pair name codes and reference sequences are shown in Table 3. The primer name code typically represents the gene to which the given primer pair is targeted. The primer pair name may include specific coordinates with respect to a reference sequence defined by an extraction of a section of sequence or defined by a GenBank gi number, or the corresponding complementary sequence of the extraction, or the entire GenBank gi number as indicated by the label “no extraction.” Where “no extraction” is indicated for a reference sequence, the coordinates of a primer pair named to the reference sequence are with respect to the GenBank gi listing. Gene abbreviations are shown in bold type in the “Gene Name” column.

Methods of primer design are well-known, and one of skill in the art will understand that the primer pairs configured to primer amplification of double stranded sequences will be configured and named using one strand of a double-stranded reference sequence. The forward primer is the primer of the pair that comprises full or partial sequence identity to the one strand of the sequence being used as a reference during design. The reverse primer is the primer of the pair that comprises reverse complementarity.

To determine the exact primer hybridization coordinates of a given pair of primers on a given bioagent nucleic acid sequence and to determine the sequences, molecular masses and base compositions of an amplification product to be obtained upon amplification of nucleic acid of a known bioagent with known sequence information in the region of interest with a given pair of primers, one with ordinary skill in bioinformatics is capable of obtaining alignments of the primers of the present invention with the GenBank gi number of the relevant nucleic acid sequence of the known bioagent. For example, the reference sequence GenBank gi numbers (Table 3) provide the identities of the sequences which can be obtained from GenBank. Alignments can be done using a bioinformatics tool such as BLASTn provided to the public by NCBI (Bethesda, Md.). Alternatively, a relevant GenBank sequence may be downloaded and imported into custom programmed or commercially available bioinformatics programs wherein the alignment can be carried out to determine the primer hybridization coordinates and the sequences, molecular masses and base compositions of the amplification product. For example, to obtain the hybridization coordinates of primer pair number 2095 (SEQ ID NOs: 456:1261), First the forward primer (SEQ ID NO: 456) is subjected to a BLASTn search on the publicly available NCBI BLAST website. “RefSeq_Genomic” is chosen as the BLAST database since the gi numbers refer to genomic sequences. The BLAST query is then performed. Among the top results returned is a match to GenBank gi number 21281729 (Accession Number NC_{—}003923). The result shown below, indicates that the forward primer hybridizes to positions 1530282 . . . 1530307 of the genomic sequence of Staphylococcus aureus subsp. aureus MW2 (represented by gi number 21281729).

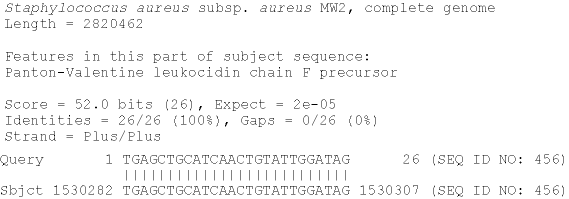

The hybridization coordinates of the reverse primer (SEQ ID NO: 1261) can be determined in a similar manner and thus, the bioagent identifying amplicon can be defined in terms of genomic coordinates. The query/subject arrangement of the result would be presented in Strand=Plus/Minus format because the reverse strand hybridizes to the reverse complement of the genomic sequence. HThe preceding sequence analyses are well known to one with ordinary skill in bioinformatics and thus, Table 3 contains sufficient information to determine the primer hybridization coordinates of any of the primers of Table 2 to the applicable reference sequences described therein.

TABLE 3
Primer Name Codes and Reference Sequence

| | | | Reference |
|---|---|---|---|
| | | | GenBank |
| Primer name | | | gi |
| code | Gene Name | Organism | number |
| RNASEP_BDP | RNase P (ribonuclease P) | Bordetella | 33591275 |
| | | pertussis | |
| RNASEP_BKM | RNase P (ribonuclease P) | Burkholderia | 53723370 |
| | | mallei | |
| RNASEP_BS | RNase P (ribonuclease P) | Bacillus | 16077068 |
| | | subtilis | |
| RNASEP_CLB | RNase P (ribonuclease P) | Clostridium | 18308982 |
| | | perfringens | |
| RNASEP_EC | RNase P (ribonuclease P) | Escherichia | 16127994 |
| | | coli | |
| RNASEP_RKP | RNase P (ribonuclease P) | Rickettsia | 15603881 |
| | | prowazekii | |
| RNASEP_SA | RNase P (ribonuclease P) | Staphylococcus | 15922990 |
| | | aureus | |
| RNASEP_VBC | RNase P (ribonuclease P) | Vibrio | 15640032 |
| | | cholerae | |
| ICD_CXB | icd (isocitrate dehydrogenase) | Coxiella | 29732244 |
| | | burnetii | |
| IS1111A | multi-locus IS1111A insertion | Acinetobacter | 29732244 |
| | element | baumannii | |
| OMPA_AY485227 | ompA (outer membrane protein A) | Rickettsia | 40287451 |
| | | prowazekii | |
| OMPB_RKP | ompB (outer membrane protein B) | Rickettsia | 15603881 |
| | | prowazekii | |
| GLTA_RKP | gltA (citrate synthase) | Vibrio | 15603881 |
| | | cholerae | |
| TOXR_VBC | toxR (transcription regulator | Francisella | 15640032 |
| | toxR) | tularensis | |
| ASD_FRT | asd (Aspartate semialdehyde | Francisella | 56707187 |
| | dehydrogenase) | tularensis | |
| GALE_FRT | galE (UDP-glucose 4-epimerase) | Shigella | 56707187 |
| | | flexneri | |
| IPAH_SGF | ipaH (invasion plasmid antigen) | Campylobacter | 30061571 |
| | | jejuni | |
| HUPB_CJ | hupB (DNA-binding protein Hu- | Coxiella | 15791399 |
| | beta) | burnetii | |
| MUPR_X75439 | mupR (mupriocin resistance | Staphylococcus | 438226 |
| | gene) | aureus | |
| PARC_X95819 | parC (topoisomerase IV) | Acinetobacter | 1212748 |
| | | baumannii | |
| SED_M28521 | sed (enterotoxin D) | Staphylococcus | 1492109 |
| | | aureus | |
| SEJ_AF053140 | sej (enterotoxin J) | Staphylococcus | 3372540 |
| | | aureus | |
| AGR- | agr-III (accessory gene | Staphylococcus | 21281729 |
| III_NC003923 | regulator-III) | aureus | |
| ARCC_NC003923 | arcC (carbamate kinase) | Staphylococcus | 21281729 |
| | | aureus | |
| AROE_NC003923 | aroE (shikimate 5-dehydrogenase | Staphylococcus | 21281729 |
| | | aureus | |
| BSA- | bsa-a (glutathione peroxidase) | Staphylococcus | 21281729 |
| A_NC003923 | | aureus | |
| BSA- | bsa-b (epidermin biosynthesis | Staphylococcus | 21281729 |
| B_NC003923 | protein EpiB) | aureus | |
| GLPF_NC003923 | glpF (glycerol transporter) | Staphylococcus | 21281729 |
| | | aureus | |
| GMK_NC003923 | gmk (guanylate kinase) | Staphylococcus | 21281729 |
| | | aureus | |
| MECI- | mecR1 (truncated methicillin | Staphylococcus | 21281729 |
| R_NC003923 | resistance protein) | aureus | |
| PTA_NC003923 | pta (phosphate | Staphylococcus | 21281729 |
| | acetyltransferase) | aureus | |
| PVLUK_NC003923 | pvluk (Panton-Valentine | Staphylococcus | 21281729 |
| | leukocidin chain F precursor) | aureus | |
| SA442_NC003923 | sa442 gene | Staphylococcus | 21281729 |
| | | aureus | |
| SEA_NC003923 | sea (staphylococcal enterotoxin | Staphylococcus | 21281729 |
| | A precursor) | aureus | |
| SEC_NC003923 | sec4 (enterotoxin type C | Staphylococcus | 21281729 |
| | precursor) | aureus | |
| TPI_NC003923 | tpi (triosephosphate isomerase) | Staphylococcus | 21281729 |
| | | aureus | |
| YQI_NC003923 | yqi (acetyl-CoA C- | Staphylococcus | 21281729 |
| | acetyltransferase homologue) | aureus | |
| AGR- | agr-II (accessory gene | Staphylococcus | 29165615 |
| II_NC002745 | regulator-II) | aureus | |
| AGR- | agr-I (accessory gene | Staphylococcus | 46019543 |
| I_AJ617706 | regulator-I) | aureus | |
| AGR- | agr-IV (accessory gene | Staphylococcus | 46019563 |
| IV_AJ617711 | regulator-III) | aureus | |
| BLAZ_NC002952 | blaZ (beta lactamase III) | Staphylococcus | 49482253 |
| | | aureus | |
| ERMA_NC002952 | ermA (rRNA methyltransferase A) | Staphylococcus | 49482253 |
| | | aureus | |
| ERMB_Y13600 | ermB (rRNA methyltransferase B) | Staphylococcus | 49482253 |
| | | aureus | |
| SEA- | sea (staphylococcal enterotoxin | Staphylococcus | 49482253 |
| SEE_NC002952 | A precursor) | aureus | |
| SEA- | sea (staphylococcal enterotoxin | Staphylococcus | 49482253 |
| SEE_NC002952 | A precursor) | aureus | |
| SEE_NC002952 | sea (staphylococcal enterotoxin | Staphylococcus | 49482253 |
| | A precursor) | aureus | |
| SEH_NC002953 | seh (staphylococcal enterotoxin | Staphylococcus | 49484912 |
| | H) | aureus | |
| ERMC_NC005908 | ermC (rRNA methyltransferase C) | Staphylococcus | 49489772 |
| | | aureus | |
| NUC_NC002758 | nuc (staphylococcal nuclease) | Staphylococcus | 15922990 |
| | | aureus | |
| SEB_NC002758 | seb (enterotoxin type B | Staphylococcus | 57634611 |
| | precursor) | aureus | |
| SEG_NC002758 | seg (staphylococcal enterotoxin | Staphylococcus | 57634611 |
| | G) | aureus | |
| SEI_NC002758 | sei (staphylococcal enterotoxin | Staphylococcus | 57634611 |
| | I) | aureus | |
| TSST_NC002758 | tsst (toxic shock syndrome | Staphylococcus | 15922990 |
| | toxin-1) | aureus | |
| TUFB_NC002758 | tufB (Elongation factor Tu) | Staphylococcus | 15922990 |
| | | aureus | |
| TSST1_NC002758.2 | tsst (toxic shock syndrome | Staphylococcus | 57634611 |
| | toxin-1) | aureus | |

Note:
artificial reference sequences represent concatenations of partial gene extractions from the indicated reference gi number. Partial sequences were used to create the concatenated sequence because complete gene sequences were not necessary for primer design.

### Example 2

### Sample Preparation and PCR

Genomic DNA was prepared from samples using the DNeasy Tissue Kit (Qiagen, Valencia, Calif.) according to the manufacturer's protocols.

All PCR reactions were assembled in 50 μL reaction volumes in a 96-well microtiter plate format using a Packard MPII liquid handling robotic platform and M.J. Dyad thermocyclers (MJ research, Waltham, Mass.) or Eppendorf Mastercycler thermocyclers (Eppendorf, Westbury, N.Y.). The PCR reaction mixture consisted of 4 units of Amplitaq Gold, 1× buffer II (Applied Biosystems, Foster City, Calif.), 1.5 mM MgCl₂, 0.4 M betaine, 800 μM dNTP mixture and 250 nM of each primer. The following typical PCR conditions were used: 95° C. for 10 min followed by 8 cycles of 95° C. for 30 seconds, 48° C. for 30 seconds, and 72° C. 30 seconds with the 48° C. annealing temperature increasing 0.9° C. with each of the eight cycles. The PCR was then continued for 37 additional cycles of 95° C. for 15 seconds, 56° C. for 20 seconds, and 72° C. 20 seconds.

### Example 3

### Purification of PCR Products for Mass Spectrometry with Ion Exchange Resin-Magnetic Beads

For solution capture of nucleic acids with ion exchange resin linked to magnetic beads, 25 μl of a 2.5 mg/mL suspension of BioClone amine terminated superparamagnetic beads were added to 25 to 50 microliters of a PCR (or RT-PCR) reaction containing approximately 10 pM of a typical PCR amplification product. The above suspension was mixed for approximately 5 minutes by vortexing or pipetting, after which the liquid was removed after using a magnetic separator. The beads containing bound PCR amplification product were then washed three times with 50 mM ammonium bicarbonate/50% MeOH or 100 mM ammonium bicarbonate/50% MeOH, followed by three more washes with 50% MeOH. The bound PCR amplicon was eluted with a solution of 25 mM piperidine, 25 mM imidazole, 35% MeOH which included peptide calibration standards.

### Example 4

### Mass Spectrometry and Base Composition Analysis

The ESI-FTICR mass spectrometer is based on a Bruker Daltonics (Billerica, Mass.) Apex II 70e electrospray ionization Fourier transform ion cyclotron resonance mass spectrometer that employs an actively shielded 7 Tesla superconducting magnet. The active shielding constrains the majority of the fringing magnetic field from the superconducting magnet to a relatively small volume. Thus, components that might be adversely affected by stray magnetic fields, such as CRT monitors, robotic components, and other electronics, can operate in close proximity to the FTICR spectrometer. All aspects of pulse sequence control and data acquisition were performed on a 600 MHz Pentium II data station running Bruker's Xmass software under Windows NT 4.0 operating system. Sample aliquots, typically 15 μl, were extracted directly from 96-well microtiter plates using a CTC HTS PAL autosampler (LEAP Technologies, Carrboro, N.C.) triggered by the FTICR data station. Samples were injected directly into a 10 μl sample loop integrated with a fluidics handling system that supplies the 100 μl/hr flow rate to the ESI source. Ions were formed via electrospray ionization in a modified Analytica (Branford, Conn.) source employing an off axis, grounded electrospray probe positioned approximately 1.5 cm from the metalized terminus of a glass desolvation capillary. The atmospheric pressure end of the glass capillary was biased at 6000 V relative to the ESI needle during data acquisition. A counter-current flow of dry N₂ was employed to assist in the desolvation process. Ions were accumulated in an external ion reservoir comprised of an rf-only hexapole, a skimmer cone, and an auxiliary gate electrode, prior to injection into the trapped ion cell where they were mass analyzed. Ionization duty cycles greater than 99% were achieved by simultaneously accumulating ions in the external ion reservoir during ion detection. Each detection event consisted of 1M data points digitized over 2.3 s. To improve the signal-to-noise ratio (S/N), 32 scans were co-added for a total data acquisition time of 74 s.

The ESI-TOF mass spectrometer is based on a Bruker Daltonics MicroTOF™. Ions from the ESI source undergo orthogonal ion extraction and are focused in a reflectron prior to detection. The TOF and FTICR are equipped with the same automated sample handling and fluidics described above. Ions are formed in the standard MicroTOF™ ESI source that is equipped with the same off-axis sprayer and glass capillary as the FTICR ESI source. Consequently, source conditions were the same as those described above. External ion accumulation was also employed to improve ionization duty cycle during data acquisition. Each detection event on the TOF was comprised of 75,000 data points digitized over 75 μs.

The sample delivery scheme allows sample aliquots to be rapidly injected into the electrospray source at high flow rate and subsequently be electrosprayed at a much lower flow rate for improved ESI sensitivity. Prior to injecting a sample, a bolus of buffer was injected at a high flow rate to rinse the transfer line and spray needle to avoid sample contamination/carryover. Following the rinse step, the autosampler injected the next sample and the flow rate was switched to low flow. Following a brief equilibration delay, data acquisition commenced. As spectra were co-added, the autosampler continued rinsing the syringe and picking up buffer to rinse the injector and sample transfer line. In general, two syringe rinses and one injector rinse were required to minimize sample carryover. During a routine screening protocol a new sample mixture was injected every 106 seconds. More recently a fast wash station for the syringe needle has been implemented which, when combined with shorter acquisition times, facilitates the acquisition of mass spectra at a rate of just under one spectrum/minute.

Raw mass spectra were post-calibrated with an internal mass standard and deconvoluted to monoisotopic molecular masses. Unambiguous base compositions were derived from the exact mass measurements of the complementary single-stranded oligonucleotides. Quantitative results are obtained by comparing the peak heights with an internal PCR calibration standard present in every PCR well at 500 molecules per well. Calibration methods are commonly owned and disclosed in U.S. Provisional Patent Application Ser. No. 60/545,425 which is incorporated herein by reference in entirety.

### Example 5

### De Novo Determination of Base Composition of Amplification Products Using Molecular Mass Modified Deoxynucleotide Triphosphates

Because the molecular masses of the four natural nucleobases have a relatively narrow molecular mass range (A=313.058, G=329.052, C=289.046, T=304.046—See Table 4), a persistent source of ambiguity in assignment of base composition can occur as follows: two nucleic acid strands having different base composition may have a difference of about 1 Da when the base composition difference between the two strands is G [symbol] A (−15.994) combined with C [symbol] T (+15.000). For example, one 99-mer nucleic acid strand having a base composition of A.sub.27G.sub.30C.sub.21T.sub.21 has a theoretical molecular mass of 30779.058 while another 99-mer nucleic acid strand having a base composition of A.sub.26G.sub.31C.sub.22T.sub.20 has a theoretical molecular mass of 30780.052. A 1 Da difference in molecular mass may be within the experimental error of a molecular mass measurement and thus, the relatively narrow molecular mass range of the four natural nucleobases imposes an uncertainty factor.

The present invention provides for a means for removing this theoretical 1 Da uncertainty factor through amplification of a nucleic acid with one mass-tagged nucleobase and three natural nucleobases. The term “nucleobase” as used herein is synonymous with other terms in use in the art including “nucleotide,” “deoxynucleotide,” “nucleotide residue,” “deoxynucleotide residue,” “nucleotide triphosphate (NTP),” or deoxynucleotide triphosphate (dNTP).

Addition of significant mass to one of the 4 nucleobases (dNTPs) in an amplification reaction, or in the primers themselves, will result in a significant difference in mass of the resulting amplification product (significantly greater than 1 Da) arising from ambiguities arising from the G [symbol] A combined with C [symbol] T event (Table 4). Thus, the same the G [symbol] A (−15.994) event combined with 5-Iodo- [symbol] T (−110.900) event would result in a molecular mass difference of 126.894. If the molecular mass of the base composition A.sub.27G.sub.30 5-Iodo-C.sub.21T.sub.21 (33422.958) is compared with A.sub.26G.sub.315-Iodo-C.sub.22T.sub.20, (33549.852) the theoretical molecular mass difference is +126.894. The experimental error of a molecular mass measurement is not significant with regard to this molecular mass difference. Furthermore, the only base composition consistent with a measured molecular mass of the 99-mer nucleic acid is A.sub.27G.sub.305-Iodo-C.sub.21T.sub.21. In contrast, the analogous amplification without the mass tag has 18 possible base compositions.

TABLE 4
Molecular Masses of Natural Nucleobases and the Mass-Modified
Nucleobase 5-Iodo-C and Molecular Mass Differences Resulting
from Transitions

| | Molecular | | Δ Molecular |
|---|---|---|---|
| Nucleobase | Mass | Transition | Mass |
| A | 313.058 | A-->T | −9.012 |
| A | 313.058 | A-->C | −24.012 |
| A | 313.058 | A-->5- | 101.888 |
| | | Iodo-C | |
| A | 313.058 | A-->G | 15.994 |
| T | 304.046 | T-->A | 9.012 |
| T | 304.046 | T-->C | −15.000 |
| T | 304.046 | T-->5- | 110.900 |
| | | Iodo-C | |
| T | 304.046 | T-->G | 25.006 |
| C | 289.046 | C-->A | 24.012 |
| C | 289.046 | C-->T | 15.000 |
| C | 289.046 | C-->G | 40.006 |
| 5-Iodo-C | 414.946 | 5-Iodo-C-->A | −101.888 |
| 5-Iodo-C | 414.946 | 5-Iodo-C-->T | −110.900 |
| 5-Iodo-C | 414.946 | 5-Iodo-C-->G | −85.894 |
| G | 329.052 | G-->A | −15.994 |
| G | 329.052 | G-->T | −25.006 |
| G | 329.052 | G-->C | −40.006 |
| G | 329.052 | G-->5- | 85.894 |
| | | Iodo-C | |

Mass spectra of bioagent-identifying amplicons were analyzed independently using a maximum-likelihood processor, such as is widely used in radar signal processing. This processor, referred to as GenX, first makes maximum likelihood estimates of the input to the mass spectrometer for each primer by running matched filters for each base composition aggregate on the input data. This includes the GenX response to a calibrant for each primer.

The algorithm emphasizes performance predictions culminating in probability-of-detection versus probability-of-false-alarm plots for conditions involving complex backgrounds of naturally occurring organisms and environmental contaminants. Matched filters consist of a priori expectations of signal values given the set of primers used for each of the bioagents. A genomic sequence database is used to define the mass base count matched filters. The database contains the sequences of known bacterial bioagents and includes threat organisms as well as benign background organisms. The latter is used to estimate and subtract the spectral signature produced by the background organisms. A maximum likelihood detection of known background organisms is implemented using matched filters and a running-sum estimate of the noise covariance. Background signal strengths are estimated and used along with the matched filters to form signatures which are then subtracted. The maximum likelihood process is applied to this “cleaned up” data in a similar manner employing matched filters for the organisms and a running-sum estimate of the noise-covariance for the cleaned up data.

The amplitudes of all base compositions of bioagent-identifying amplicons for each primer are calibrated and a final maximum likelihood amplitude estimate per organism is made based upon the multiple single primer estimates. Models of all system noise are factored into this two-stage maximum likelihood calculation. The processor reports the number of molecules of each base composition contained in the spectra. The quantity of amplification product corresponding to the appropriate primer set is reported as well as the quantities of primers remaining upon completion of the amplification reaction.

Base count blurring can be carried out as follows. “Electronic PCR” can be conducted on nucleotide sequences of the desired bioagents to obtain the different expected base counts that could be obtained for each primer pair. See for example, ncbi.nlm.nih.gov/sutils/e-per/; Schuler, Genome Res. 7:541-50, 1997. In one illustrative embodiment, one or more spreadsheets, such as Microsoft Excel workbooks contain a plurality of worksheets. First in this example, there is a worksheet with a name similar to the workbook name; this worksheet contains the raw electronic PCR data. Second, there is a worksheet named “filtered bioagents base count” that contains bioagent name and base count; there is a separate record for each strain after removing sequences that are not identified with a genus and species and removing all sequences for bioagents with less than 10 strains. Third, there is a worksheet, “Sheet1” that contains the frequency of substitutions, insertions, or deletions for this primer pair. This data is generated by first creating a pivot table from the data in the “filtered bioagents base count” worksheet and then executing an Excel VBA macro. The macro creates a table of differences in base counts for bioagents of the same species, but different strains. One of ordinary skill in the art may understand additional pathways for obtaining similar table differences without undo experimentation.

Application of an exemplary script, involves the user defining a threshold that specifies the fraction of the strains that are represented by the reference set of base counts for each bioagent. The reference set of base counts for each bioagent may contain as many different base counts as are needed to meet or exceed the threshold. The set of reference base counts is defined by taking the most abundant strain's base type composition and adding it to the reference set and then the next most abundant strain's base type composition is added until the threshold is met or exceeded. The current set of data was obtained using a threshold of 55%, which was obtained empirically.

For each base count not included in the reference base count set for that bioagent, the script then proceeds to determine the manner in which the current base count differs from each of the base counts in the reference set. This difference may be represented as a combination of substitutions, Si=Xi, and insertions, Ii=Yi, or deletions, Di=Zi. If there is more than one reference base count, then the reported difference is chosen using rules that aim to minimize the number of changes and, in instances with the same number of changes, minimize the number of insertions or deletions. Therefore, the primary rule is to identify the difference with the minimum sum (Xi+Yi) or (Xi+Zi), e.g., one insertion rather than two substitutions. If there are two or more differences with the minimum sum, then the one that will be reported is the one that contains the most substitutions.

Differences between a base count and a reference composition are categorized as one, two, or more substitutions, one, two, or more insertions, one, two, or more deletions, and combinations of substitutions and insertions or deletions. The different classes of nucleobase changes and their probabilities of occurrence have been delineated in U.S. Patent Application Publication No. 2004209260 which is incorporated herein by reference in entirety.

### Example 6

### Use of Broad Range Survey and Division Wide Primer Pairs for Identification of Bacteria in an Epidemic Surveillance Investigation

This investigation employed a set of 16 primer pairs which is herein designated the “surveillance primer set” and comprises broad range survey primer pairs, division wide primer pairs and a single Bacillus clade primer pair. The surveillance primer set is shown in Table 5 and consists of primer pairs originally listed in Table 2. This surveillance set comprises primers with T modifications (note TMOD designation in primer names) which constitutes a functional improvement with regard to prevention of non-templated adenylation (vide supra) relative to originally selected primers which are displayed below in the same row. Primer pair 449 (non-T modified) has been modified twice. Its predecessors are primer pairs 70 and 357, displayed below in the same row. Primer pair 360 has also been modified twice and its predecessors are primer pairs 17 and 118.

TABLE 5
Bacterial Primer Pairs of the Surveillance Primer Set

| | | Forward | | Reverse | |
|---|---|---|---|---|---|
| Primer | | Primer | | Primer | |
| Pair | | (SEQ ID | | (SEQ ID | |
| No. | Forward Primer Name | NO:) | Reverse Primer Name | NO:) | Target Gene |
| 346 | 16S_EC_713_732_TMOD_F | 202 | 16S_EC_789_809_TMOD_R | 1110 | 16S rRNA |
| 10 | 16S_EC_713_732_F | 21 | 16S_EC_789_809 | 798 | 16S rRNA |
| 347 | 16S_EC_785_806_TMOD_F | 560 | 16S_EC_880_897_TMOD_R | 1278 | 16S rRNA |
| 11 | 16S_EC_785_806_F | 118 | 16S_EC_880_897_R | 830 | 16S rRNA |
| 348 | 16S_EC_960_981_TMOD_F | 706 | 16S_EC_1054_1073_TMOD_R | 895 | 16S rRNA |
| 14 | 16S_EC_960_981_F | 672 | 16S_EC_1054_1073_R | 735 | 16S rRNA |
| 349 | 23S_EC_1826_1843_TMOD_F | 401 | 23S_EC_1906_1924_TMOD_R | 1156 | 23S rRNA |
| 16 | 23S_EC_1826_1843_F | 80 | 23S_EC_1906_1924_R | 805 | 23S rRNA |
| 352 | INFB_EC_1365_1393_TMOD_F | 687 | INFB_EC_1439_1467_TMOD_R | 1411 | infB |
| 34 | INFB_EC_1365_1393_F | 524 | INFB_EC_1439_1467_R | 1248 | infB |
| 354 | RPOC_EC_2218_2241_TMOD_F | 405 | RPOC_EC_2313_2337_TMOD_R | 1072 | rpoC |
| 52 | RPOC_EC_2218_2241_F | 81 | RPOC_EC_2313_2337_R | 790 | rpoC |
| 355 | SSPE_BA_115_137_TMOD_F | 255 | SSPE_BA_197_222_TMOD_R | 1402 | sspE |
| 58 | SSPE_BA_115_137_F | 45 | SSPE_BA_197_222_R | 1201 | sspE |
| 356 | RPLB_EC_650_679_TMOD_F | 232 | RPLB_EC_739_762_TMOD_R | 592 | rplB |
| 66 | RPLB_EC_650_679_F | 98 | RPLB_EC_739_762_R | 999 | rplB |
| 358 | VALS_EC_1105_1124_TMOD_F | 385 | VALS_EC_1195_1218_TMOD_R | 1093 | valS |
| 71 | VALS_EC_1105_1124_F | 77 | VALS_EC_1195_1218_R | 795 | valS |
| 359 | RPOB_EC_1845_1866_TMOD_F | 659 | RPOB_EC_1909_1929_TMOD_R | 1250 | rpoB |
| 72 | RPOB_EC_1845_1866_F | 233 | RPOB_EC_1909_1929_R | 825 | rpoB |
| 360 | 23S_EC_2646_2667_TMOD_F | 409 | 23S_EC_2745_2765_TMOD_R | 1434 | 23S rRNA |
| 118 | 23S_EC_2646_2667_F | 84 | 23S_EC_2745_2765_R | 1389 | 23S rRNA |
| 17 | 23S_EC_2645_2669_F | 408 | 23S_EC_2744_2761_R | 1252 | 23S rRNA |
| 361 | 16S_EC_1090_1111_2_TMOD_F | 697 | 16S_EC_1175_1196_TMOD_R | 1398 | 16S rRNA |
| 3 | 16S_EC_1090_1111_2_F | 651 | 16S_EC_1175_1196_R | 1159 | 16S rRNA |
| 362 | RPOB_EC_3799_3821_TMOD_F | 581 | RPOB_EC_3862_3888_TMOD_R | 1325 | rpoB |
| 289 | RPOB_EC_3799_3821_F | 124 | RPOB_EC_3862_3888_R | 840 | rpoB |
| 363 | RPOC_EC_2146_2174_TMOD_F | 284 | RPOC_EC_2227_2245_TMOD_R | 898 | rpoC |
| 290 | RPOC_EC_2146_2174_F | 52 | RPOC_EC_2227_2245_R | 736 | rpoC |
| 367 | TUFB_EC_957_979_TMOD_F | 308 | TUFB_EC_1034_1058_TMOD_R | 1276 | tufB |
| 293 | TUFB_EC_957_979_F | 55 | TUFB_EC_1034_1058_R | 829 | tufB |
| 449 | RPLB_EC_690_710_F | 309 | RPLB_EC_737_758_R | 1336 | rplB |
| 357 | RPLB_EC_688_710_TMOD_F | 296 | RPLB_EC_736_757_TMOD_R | 1337 | rplB |
| 67 | RPLB_EC_688_710_F | 54 | RPLB_EC_736_757_R | 842 | rplB |

The 16 primer pairs of the surveillance set are used to produce bioagent identifying amplicons whose base compositions are sufficiently different amongst all known bacteria at the species level to identify, at a reasonable confidence level, any given bacterium at the species level. As shown in Tables 6A-E, common respiratory bacterial pathogens can be distinguished by the base compositions of bioagent identifying amplicons obtained using the 16 primer pairs of the surveillance set. In some cases, triangulation identification improves the confidence level for species assignment. For example, nucleic acid from Streptococcus pyogenes can be amplified by nine of the sixteen surveillance primer pairs and Streptococcus pneumoniae can be amplified by ten of the sixteen surveillance primer pairs. The base compositions of the bioagent identifying amplicons are identical for only one of the analogous bioagent identifying amplicons and differ in all of the remaining analogous bioagent identifying amplicons by up to four bases per bioagent identifying amplicon. The resolving power of the surveillance set was confirmed by determination of base compositions for 120 isolates of respiratory pathogens representing 70 different bacterial species and the results indicated that natural variations (usually only one or two base substitutions per bioagent identifying amplicon) amongst multiple isolates of the same species did not prevent correct identification of major pathogenic organisms at the species level.

Bacillus anthracis is a well known biological warfare agent which has emerged in domestic terrorism in recent years. Since it was envisioned to produce bioagent identifying amplicons for identification of Bacillus anthracis, additional drill-down analysis primers were configured to target genes present on virulence plasmids of Bacillus anthracis so that additional confidence could be reached in positive identification of this pathogenic organism. Three drill-down analysis primers were configured and are listed in Tables 2 and 6. In Table 6, the drill-down set comprises primers with T modifications (note TMOD designation in primer names) which constitutes a functional improvement with regard to prevention of non-templated adenylation (vide supra) relative to originally selected primers which are displayed below in the same row.

TABLE 6
Drill-Down Primer Pairs for Confirmation of Identification of Bacillus anthracis

| | | Forward | | Reverse | |
|---|---|---|---|---|---|
| Primer | | Primer | | Primer | |
| Pair | | (SEQ ID | | (SEQ ID | |
| No. | Forward Primer Name | NO:) | Reverse Primer Name | NO:) | Target Gene |
| 350 | CAPC_BA_274_303_TMOD_F | 476 | CAPC_BA_349_376_TMOD_R | 1314 | capC |
| 24 | CAPC_BA_274_303_F | 109 | CAPC_BA_349_376_R | 837 | capC |
| 351 | CYA_BA_1353_1379_TMOD_F | 355 | CYA_BA_1448_1467_TMOD_R | 1423 | cyA |
| 30 | CYA_BA_1353_1379_F | 64 | CYA_BA_1448_1467_R | 1342 | cyA |
| 353 | LEF_BA_756_781_TMOD_F | 220 | LEF_BA_843_872_TMOD_R | 1394 | lef |
| 37 | LEF_BA_756_781_F | 26 | LEF_BA_843_872_R | 1135 | lef |

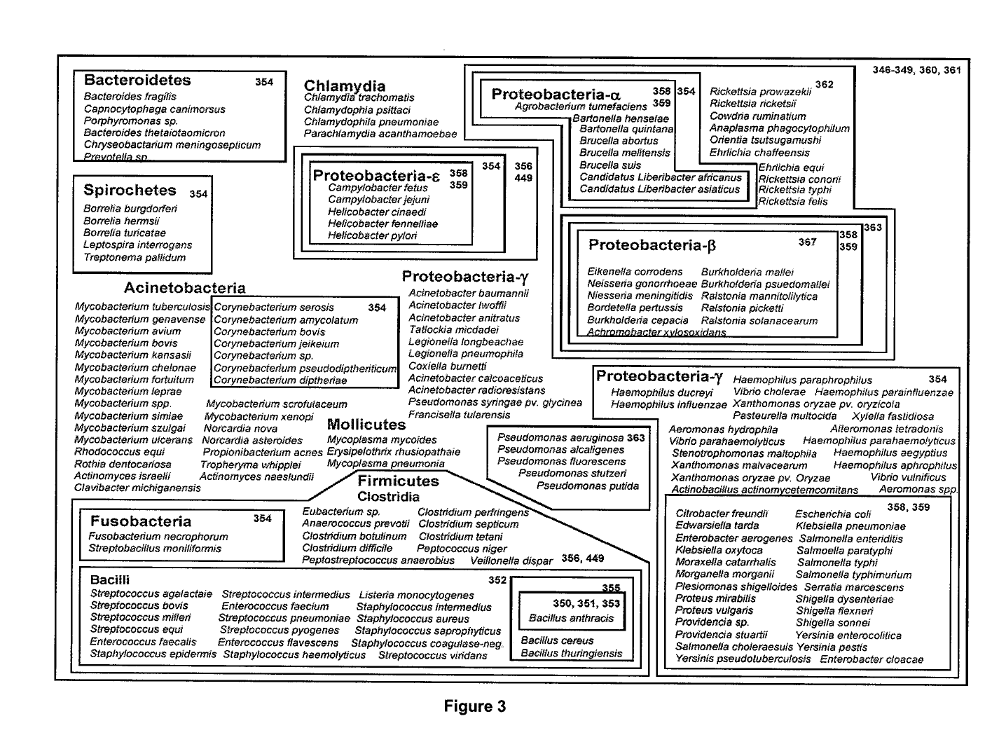

Phylogenetic coverage of bacterial space of the sixteen surveillance primers of Table 5 and the three Bacillus anthracis drill-down primers of Table 6 is shown in FIG. 3 which lists common pathogenic bacteria. FIG. 3 is not meant to be comprehensive in illustrating all species identified by the primers. Only pathogenic bacteria are listed as representative examples of the bacterial species that can be identified by the primers and methods of the present invention. Nucleic acid of groups of bacteria enclosed within the polygons of FIG. 3 can be amplified to obtain bioagent identifying amplicons using the primer pair numbers listed in the upper right hand corner of each polygon. Primer coverage for polygons within polygons is additive. As an illustrative example, bioagent identifying amplicons can be obtained for Chlamydia trachomatis by amplification with, for example, primer pairs 346-349, 360 and 361, but not with any of the remaining primers of the surveillance primer set. On the other hand, bioagent identifying amplicons can be obtained from nucleic acid originating from Bacillus anthracis (located within 5 successive polygons) using, for example, any of the following primer pairs: 346-349, 360, 361 (base polygon), 356, 449 (second polygon), 352 (third polygon), 355 (fourth polygon), 350, 351 and 353 (fifth polygon). Multiple coverage of a given organism with multiple primers provides for increased confidence level in identification of the organism as a result of enabling broad triangulation identification.

In Tables 7A-E, base compositions of respiratory pathogens for primer target regions are shown. Two entries in a cell, represent variation in ribosomal DNA operons. The most predominant base composition is shown first and the minor (frequently a single operon) is indicated by an asterisk (*). Entries with NO DATA mean that the primer would not be expected to prime this species due to mismatches between the primer and target region, as determined by theoretical PCR.

TABLE 7A
Base Compositions of Common Respiratory Pathogens for Bioagent Identifying
Amplicons Corresponding to Primer Pair Nos: 346, 347 and 348

| | | Primer 346 | Primer 347 | Primer 348 |
|---|---|---|---|---|
| Organism | Strain | [A G C T] | [A G C T] | [A G C T] |
| Klebsiella | MGH78578 | [29 32 25 13] | [23 38 28 26] | [26 32 28 30] |
| pneumoniae | | [29 31 25 13]* | [23 37 28 26]* | [26 31 28 30]* |
| Yersinia pestis | CO-92 Biovar | [29 32 25 13] | [22 39 28 26] | [29 30 28 29] |
| | Orientalis | | | [30 30 27 29]* |
| Yersinia pestis | KIM5 P12 (Biovar | [29 32 25 13] | [22 39 28 26] | [29 30 28 29] |
| | Mediaevalis) | | | |
| Yersinia pestis | 91001 | [29 32 25 13] | [22 39 28 26] | [29 30 28 29] |
| | | | | [30 30 27 29]* |
| Haemophilus | KW20 | [28 31 23 17] | [24 37 25 27] | [29 30 28 29] |
| influenzae | | | | |
| Pseudomonas | PAO1 | [30 31 23 15] | [26 36 29 24] | [26 32 29 29] |
| aeruginosa | | | [27 36 29 23]* | |
| Pseudomonas | Pf0-1 | [30 31 23 15] | [26 35 29 25] | [28 31 28 29] |
| fluorescens | | | | |
| Pseudomonas | KT2440 | [30 31 23 15] | [28 33 27 27] | [27 32 29 28] |
| putida | | | | |
| Legionella | Philadelphia-1 | [30 30 24 15] | [33 33 23 27] | [29 28 28 31] |
| pneumophila | | | | |
| Francisella | schu 4 | [32 29 22 16] | [28 38 26 26] | [25 32 28 31] |
| tularensis | | | | |
| Bordetella | Tohama I | [30 29 24 16] | [23 37 30 24] | [30 32 30 26] |
| pertussis | | | | |
| Burkholderia | J2315 | [29 29 27 14] | [27 32 26 29] | [27 36 31 24] |
| cepacia | | | | [20 42 35 19]* |
| Burkholderia | K96243 | [29 29 27 14] | [27 32 26 29] | [27 36 31 24] |
| pseudomallei | | | | |
| Neisseria | FA 1090, ATCC | [29 28 24 18] | [27 34 26 28] | [24 36 29 27] |
| gonorrhoeae | 700825 | | | |
| Neisseria | MC58 (serogroup B) | [29 28 26 16] | [27 34 27 27] | [25 35 30 26] |
| meningitidis | | | | |
| Neisseria | serogroup C, FAM18 | [29 28 26 16] | [27 34 27 27] | [25 35 30 26] |
| meningitidis | | | | |
| Neisseria | Z2491 (serogroup A) | [29 28 26 16] | [27 34 27 27] | [25 35 30 26] |
| meningitidis | | | | |
| Chlamydophila | TW-183 | [31 27 22 19] | NO DATA | [32 27 27 29] |
| pneumoniae | | | | |
| Chlamydophila | AR39 | [31 27 22 19] | NO DATA | [32 27 27 29] |
| pneumoniae | | | | |
| Chlamydophila | CWL029 | [31 27 22 19] | NO DATA | [32 27 27 29] |
| pneumoniae | | | | |
| Chlamydophila | J138 | [31 27 22 19] | NO DATA | [32 27 27 29] |
| pneumoniae | | | | |
| Corynebacterium | NCTC13129 | [29 34 21 15] | [22 38 31 25] | [22 33 25 34] |
| diphtheriae | | | | |
| Mycobacterium | k10 | [27 36 21 15] | [22 37 30 28] | [21 36 27 30] |
| avium | | | | |
| Mycobacterium | 104 | [27 36 21 15] | [22 37 30 28] | [21 36 27 30] |
| avium | | | | |
| Mycobacterium | CSU#93 | [27 36 21 15] | [22 37 30 28] | [21 36 27 30] |
| tuberculosis | | | | |
| Mycobacterium | CDC 1551 | [27 36 21 15] | [22 37 30 28] | [21 36 27 30] |
| tuberculosis | | | | |
| Mycobacterium | H37Rv (lab strain) | [27 36 21 15] | [22 37 30 28] | [21 36 27 30] |
| tuberculosis | | | | |
| Mycoplasma | M129 | [31 29 19 20] | NO DATA | NO DATA |
| pneumoniae | | | | |
| Staphylococcus | MRSA252 | [27 30 21 21] | [25 35 30 26] | [30 29 30 29] |
| aureus | | | | [29 31 30 29]* |
| Staphylococcus | MSSA476 | [27 30 21 21] | [25 35 30 26] | [30 29 30 29] |
| aureus | | | | [30 29 29 30]* |
| Staphylococcus | COL | [27 30 21 21] | [25 35 30 26] | [30 29 30 29] |
| aureus | | | | [30 29 29 30]* |
| Staphylococcus | Mu50 | [27 30 21 21] | [25 35 30 26] | [30 29 30 29] |
| aureus | | | | [30 29 29 30]* |
| Staphylococcus | MW2 | [27 30 21 21] | [25 35 30 26] | [30 29 30 29] |
| aureus | | | | [30 29 29 30]* |
| Staphylococcus | N315 | [27 30 21 21] | [25 35 30 26] | [30 29 30 29] |
| aureus | | | | [30 29 29 30]* |
| Staphylococcus | NCTC 8325 | [27 30 21 21] | [25 35 30 26] | [30 29 30 29] |
| aureus | | | [25 35 31 26]* | [30 29 29 30] |
| Streptococcus | NEM316 | [26 32 23 18] | [24 36 31 25] | [25 32 29 30] |
| agalactiae | | | [24 36 30 26]* | |
| Streptococcus | NC_002955 | [26 32 23 18] | [23 37 31 25] | [29 30 25 32] |
| equi | | | | |
| Streptococcus | MGAS8232 | [26 32 23 18] | [24 37 30 25] | [25 31 29 31] |
| pyogenes | | | | |
| Streptococcus | MGAS315 | [26 32 23 18] | [24 37 30 25] | [25 31 29 31] |
| pyogenes | | | | |
| Streptococcus | SSI-1 | [26 32 23 18] | [24 37 30 25] | [25 31 29 31] |
| pyogenes | | | | |
| Streptococcus | MGAS10394 | [26 32 23 18] | [24 37 30 25] | [25 31 29 31] |
| pyogenes | | | | |
| Streptococcus | Manfredo (M5) | [26 32 23 18] | [24 37 30 25] | [25 31 29 31] |
| pyogenes | | | | |
| Streptococcus | SF370 (M1) | [26 32 23 18] | [24 37 30 25] | [25 31 29 31] |
| pyogenes | | | | |
| Streptococcus | 670 | [26 32 23 18] | [25 35 28 28] | [25 32 29 30] |
| pneumoniae | | | | |
| Streptococcus | R6 | [26 32 23 18] | [25 35 28 28] | [25 32 29 30] |
| pneumoniae | | | | |
| Streptococcus | TIGR4 | [26 32 23 18] | [25 35 28 28] | [25 32 30 29] |
| pneumoniae | | | | |
| Streptococcus | NCTC7868 | [25 33 23 18] | [24 36 31 25] | [25 31 29 31] |
| gordonii | | | | |
| Streptococcus | NCTC 12261 | [26 32 23 18] | [25 35 30 26] | [25 32 29 30] |
| mitis | | | | [24 31 35 29]* |
| Streptococcus | UA159 | [24 32 24 19] | [25 37 30 24] | [28 31 26 31] |
| mutans | | | | |

TABLE 7B
Base Compositions of Common Respiratory Pathogens for Bioagent Identifying
Amplicons Corresponding to Primer Pair Nos: 349, 360, and 356

| | | Primer 349 | Primer 360 | Primer 356 |
|---|---|---|---|---|
| Organism | Strain | [A G C T] | [A G C T] | [A G C T] |
| Klebsiella | MGH78578 | [25 31 25 22] | [33 37 25 27] | NO DATA |
| pneumoniae | | | | |
| Yersinia pestis | CO-92 Biovar | [25 31 27 20] | [34 35 25 28] | NO DATA |
| | Orientalis | [25 32 26 20]* | | |
| Yersinia pestis | KIM5 P12 (Biovar | [25 31 27 20] | [34 35 25 28] | NO DATA |
| | Mediaevalis) | [25 32 26 20]* | | |
| Yersinia pestis | 91001 | [25 31 27 20] | [34 35 25 28] | NO DATA |
| Haemophilus | KW20 | [28 28 25 20] | [32 38 25 27] | NO DATA |
| influenzae | | | | |
| Pseudomonas | PAO1 | [24 31 26 20] | [31 36 27 27] | NO DATA |
| aeruginosa | | | [31 36 27 28]* | |
| Pseudomonas | Pf0-1 | NO DATA | [30 37 27 28] | NO DATA |
| fluorescens | | | [30 37 27 28] | |
| Pseudomonas | KT2440 | [24 31 26 20] | [30 37 27 28] | NO DATA |
| putida | | | | |
| Legionella | Philadelphia-1 | [23 30 25 23] | [30 39 29 24] | NO DATA |
| pneumophila | | | | |
| Francisella | schu 4 | [26 31 25 19] | [32 36 27 27] | NO DATA |
| tularensis | | | | |
| Bordetella | Tohama I | [21 29 24 18] | [33 36 26 27] | NO DATA |
| pertussis | | | | |
| Burkholderia | J2315 | [23 27 22 20] | [31 37 28 26] | NO DATA |
| cepacia | | | | |
| Burkholderia | K96243 | [23 27 22 20] | [31 37 28 26] | NO DATA |
| pseudomallei | | | | |
| Neisseria | FA 1090, ATCC 700825 | [24 27 24 17] | [34 37 25 26] | NO DATA |
| gonorrhoeae | | | | |
| Neisseria | MC58 (serogroup B) | [25 27 22 18] | [34 37 25 26] | NO DATA |
| meningitidis | | | | |
| Neisseria | serogroup C, FAM18 | [25 26 23 18] | [34 37 25 26] | NO DATA |
| meningitidis | | | | |
| Neisseria | Z2491 (serogroup A) | [25 26 23 18] | [34 37 25 26] | NO DATA |
| meningitidis | | | | |
| Chlamydophila | TW-183 | [30 28 27 18] | NO DATA | NO DATA |
| pneumoniae | | | | |
| Chlamydophila | AR39 | [30 28 27 18] | NO DATA | NO DATA |
| pneumoniae | | | | |
| Chlamydophila | CWL029 | [30 28 27 18] | NO DATA | NO DATA |
| pneumoniae | | | | |
| Chlamydophila | J138 | [30 28 27 18] | NO DATA | NO DATA |
| pneumoniae | | | | |
| Corynebacterium | NCTC13129 | NO DATA | [29 40 28 25] | NO DATA |
| diphtheriae | | | | |
| Mycobacterium | k10 | NO DATA | [33 35 32 22] | NO DATA |
| avium | | | | |
| Mycobacterium | 104 | NO DATA | [33 35 32 22] | NO DATA |
| avium | | | | |
| Mycobacterium | CSU#93 | NO DATA | [30 36 34 22] | NO DATA |
| tuberculosis | | | | |
| Mycobacterium | CDC 1551 | NO DATA | [30 36 34 22] | NO DATA |
| tuberculosis | | | | |
| Mycobacterium | H37Rv (lab strain) | NO DATA | [30 36 34 22] | NO DATA |
| tuberculosis | | | | |
| Mycoplasma | M129 | [28 30 24 19] | [34 31 29 28] | NO DATA |
| pneumoniae | | | | |
| Staphylococcus | MRSA252 | [26 30 25 20] | [31 38 24 29] | [33 30 31 27] |
| aureus | | | | |
| Staphylococcus | MSSA476 | [26 30 25 20] | [31 38 24 29] | [33 30 31 27] |
| aureus | | | | |
| Staphylococcus | COL | [26 30 25 20] | [31 38 24 29] | [33 30 31 27] |
| aureus | | | | |
| Staphylococcus | Mu50 | [26 30 25 20] | [31 38 24 29] | [33 30 31 27] |
| aureus | | | | |
| Staphylococcus | MW2 | [26 30 25 20] | [31 38 24 29] | [33 30 31 27] |
| aureus | | | | |
| Staphylococcus | N315 | [26 30 25 20] | [31 38 24 29] | [33 30 31 27] |
| aureus | | | | |
| Staphylococcus | NCTC 8325 | [26 30 25 20] | [31 38 24 29] | [33 30 31 27] |
| aureus | | | | |
| Streptococcus | NEM316 | [28 31 22 20] | [33 37 24 28] | [37 30 28 26] |
| agalactiae | | | | |
| Streptococcus | NC_002955 | [28 31 23 19] | [33 38 24 27] | [37 31 28 25] |
| equi | | | | |
| Streptococcus | MGAS8232 | [28 31 23 19] | [33 37 24 28] | [38 31 29 23] |
| pyogenes | | | | |
| Streptococcus | MGAS315 | [28 31 23 19] | [33 37 24 28] | [38 31 29 23] |
| pyogenes | | | | |
| Streptococcus | SSI-1 | [28 31 23 19] | [33 37 24 28] | [38 31 29 23] |
| pyogenes | | | | |
| Streptococcus | MGAS10394 | [28 31 23 19] | [33 37 24 28] | [38 31 29 23] |
| pyogenes | | | | |
| Streptococcus | Manfredo (M5) | [28 31 23 19] | [33 37 24 28] | [38 31 29 23] |
| pyogenes | | | | |
| Streptococcus | SF370 (M1) | [28 31 23 19] | [33 37 24 28] | [38 31 29 23] |
| pyogenes | | [28 31 22 20]* | | |
| Streptococcus | 670 | [28 31 22 20] | [34 36 24 28] | [37 30 29 25] |
| pneumoniae | | | | |
| Streptococcus | R6 | [28 31 22 20] | [34 36 24 28] | [37 30 29 25] |
| pneumoniae | | | | |
| Streptococcus | TIGR4 | [28 31 22 20] | [34 36 24 28] | [37 30 29 25] |
| pneumoniae | | | | |
| Streptococcus | NCTC7868 | [28 32 23 20] | [34 36 24 28] | [36 31 29 25] |
| gordonii | | | | |
| Streptococcus | NCTC 12261 | [28 31 22 20] | [34 36 24 28] | [37 30 29 25] |
| mitis | | [29 30 22 20]* | | |
| Streptococcus | UA159 | [26 32 23 22] | [34 37 24 27] | NO DATA |
| mutans | | | | |

TABLE 7C
Base Compositions of Common Respiratory Pathogens for Bioagent Identifying
Amplicons Corresponding to Primer Pair Nos: 449, 354, and 352

| | | Primer 449 | Primer 354 | Primer 352 |
|---|---|---|---|---|
| Organism | Strain | [A G C T] | [A G C T] | [A G C T] |
| Klebsiella | MGH78578 | NO DATA | [27 33 36 26] | NO DATA |
| pneumoniae | | | | |
| Yersinia pestis | CO-92 Biovar | NO DATA | [29 31 33 29] | [32 28 20 25] |
| | Orientalis | | | |
| Yersinia pestis | KIM5 P12 (Biovar | NO DATA | [29 31 33 29] | [32 28 20 25] |
| | Mediaevalis) | | | |
| Yersinia pestis | 91001 | NO DATA | [29 31 33 29] | NO DATA |
| Haemophilus | KW20 | NO DATA | [30 29 31 32] | NO DATA |
| influenzae | | | | |
| Pseudomonas | PAO1 | NO DATA | [26 33 39 24] | NO DATA |
| aeruginosa | | | | |
| Pseudomonas | Pf0-1 | NO DATA | [26 33 34 29] | NO DATA |
| fluorescens | | | | |
| Pseudomonas | KT2440 | NO DATA | [25 34 36 27] | NO DATA |
| putida | | | | |
| Legionella | Philadelphia-1 | NO DATA | NO DATA | NO DATA |
| pneumophila | | | | |
| Francisella | schu 4 | NO DATA | [33 32 25 32] | NO DATA |
| tularensis | | | | |
| Bordetella | Tohama I | NO DATA | [26 33 39 24] | NO DATA |
| pertussis | | | | |
| Burkholderia | J2315 | NO DATA | [25 37 33 27] | NO DATA |
| cepacia | | | | |
| Burkholderia | K96243 | NO DATA | [25 37 34 26] | NO DATA |
| pseudomallei | | | | |
| Neisseria | FA 1090, ATCC 700825 | [17 23 22 10] | [29 31 32 30] | NO DATA |
| gonorrhoeae | | | | |
| Neisseria | MC58 (serogroup B) | NO DATA | [29 30 32 31] | NO DATA |
| meningitidis | | | | |
| Neisseria | serogroup C, FAM18 | NO DATA | [29 30 32 31] | NO DATA |
| meningitidis | | | | |
| Neisseria | Z2491 (serogroup A) | NO DATA | [29 30 32 31] | NO DATA |
| meningitidis | | | | |
| Chlamydophila | TW-183 | NO DATA | NO DATA | NO DATA |
| pneumoniae | | | | |
| Chlamydophila | AR39 | NO DATA | NO DATA | NO DATA |
| pneumoniae | | | | |
| Chlamydophila | CWL029 | NO DATA | NO DATA | NO DATA |
| pneumoniae | | | | |
| Chlamydophila | J138 | NO DATA | NO DATA | NO DATA |
| pneumoniae | | | | |
| Corynebacterium | NCTC13129 | NO DATA | NO DATA | NO DATA |
| diphtheriae | | | | |
| Mycobacterium | k10 | NO DATA | NO DATA | NO DATA |
| avium | | | | |
| Mycobacterium | 104 | NO DATA | NO DATA | NO DATA |
| avium | | | | |
| Mycobacterium | CSU#93 | NO DATA | NO DATA | NO DATA |
| tuberculosis | | | | |
| Mycobacterium | CDC 1551 | NO DATA | NO DATA | NO DATA |
| tuberculosis | | | | |
| Mycobacterium | H37Rv (lab strain) | NO DATA | NO DATA | NO DATA |
| tuberculosis | | | | |
| Mycoplasma | M129 | NO DATA | NO DATA | NO DATA |
| pneumoniae | | | | |
| Staphylococcus | MRSA252 | [17 20 21 17] | [30 27 30 35] | [36 24 19 26] |
| aureus | | | | |
| Staphylococcus | MSSA476 | [17 20 21 17] | [30 27 30 35] | [36 24 19 26] |
| aureus | | | | |
| Staphylococcus | COL | [17 20 21 17] | [30 27 30 35] | [35 24 19 27] |
| aureus | | | | |
| Staphylococcus | Mu50 | [17 20 21 17] | [30 27 30 35] | [36 24 19 26] |
| aureus | | | | |
| Staphylococcus | MW2 | [17 20 21 17] | [30 27 30 35] | [36 24 19 26] |
| aureus | | | | |
| Staphylococcus | N315 | [17 20 21 17] | [30 27 30 35] | [36 24 19 26] |
| aureus | | | | |
| Staphylococcus | NCTC 8325 | [17 20 21 17] | [30 27 30 35] | [35 24 19 27] |
| aureus | | | | |
| Streptococcus | NEM316 | [22 20 19 14] | [26 31 27 38] | [29 26 22 28] |
| agalactiae | | | | |
| Streptococcus | NC_002955 | [22 21 19 13] | NO DATA | NO DATA |
| equi | | | | |
| Streptococcus | MGAS8232 | [23 21 19 12] | [24 32 30 36] | NO DATA |
| pyogenes | | | | |
| Streptococcus | MGAS315 | [23 21 19 12] | [24 32 30 36] | NO DATA |
| pyogenes | | | | |
| Streptococcus | SSI-1 | [23 21 19 12] | [24 32 30 36] | NO DATA |
| pyogenes | | | | |
| Streptococcus | MGAS10394 | [23 21 19 12] | [24 32 30 36] | NO DATA |
| pyogenes | | | | |
| Streptococcus | Manfredo (M5) | [23 21 19 12] | [24 32 30 36] | NO DATA |
| pyogenes | | | | |
| Streptococcus | SF370 (M1) | [23 21 19 12] | [24 32 30 36] | NO DATA |
| pyogenes | | | | |
| Streptococcus | 670 | [22 20 19 14] | [25 33 29 35] | [30 29 21 25] |
| pneumoniae | | | | |
| Streptococcus | R6 | [22 20 19 14] | [25 33 29 35] | [30 29 21 25] |
| pneumoniae | | | | |
| Streptococcus | TIGR4 | [22 20 19 14] | [25 33 29 35] | [30 29 21 25] |
| pneumoniae | | | | |
| Streptococcus | NCTC7868 | [21 21 19 14] | NO DATA | [29 26 22 28] |
| gordonii | | | | |
| Streptococcus | NCTC 12261 | [22 20 19 14] | [26 30 32 34] | NO DATA |
| mitis | | | | |
| Streptococcus | UA159 | NO DATA | NO DATA | NO DATA |
| mutans | | | | |

TABLE 7D
Base Compositions of Common Respiratory Pathogens for Bioagent Identifying
Amplicons Corresponding to Primer Pair Nos: 355, 358, and 359

| | | Primer 355 | Primer 358 | Primer 359 |
|---|---|---|---|---|
| Organism | Strain | [A G C T] | [A G C T] | [A G C T] |
| Klebsiella | MGH78578 | NO DATA | [24 39 33 20] | [25 21 24 17] |
| pneumoniae | | | | |
| Yersinia pestis | CO-92 Biovar | NO DATA | [26 34 35 21] | [23 23 19 22] |
| | Orientalis | | | |
| Yersinia pestis | KIM5 P12 (Biovar | NO DATA | [26 34 35 21] | [23 23 19 22] |
| | Mediaevalis) | | | |
| Yersinia pestis | 91001 | NO DATA | [26 34 35 21] | [23 23 19 22] |
| Haemophilus | KW20 | NO DATA | NO DATA | NO DATA |
| influenzae | | | | |
| Pseudomonas | PAO1 | NO DATA | NO DATA | NO DATA |
| aeruginosa | | | | |
| Pseudomonas | Pf0-1 | NO DATA | NO DATA | NO DATA |
| fluorescens | | | | |
| Pseudomonas | KT2440 | NO DATA | [21 37 37 21] | NO DATA |
| putida | | | | |
| Legionella | Philadelphia-1 | NO DATA | NO DATA | NO DATA |
| pneumophila | | | | |
| Francisella | schu 4 | NO DATA | NO DATA | NO DATA |
| tularensis | | | | |
| Bordetella | Tohama I | NO DATA | NO DATA | NO DATA |
| pertussis | | | | |
| Burkholderia | J2315 | NO DATA | NO DATA | NO DATA |
| cepacia | | | | |
| Burkholderia | K96243 | NO DATA | NO DATA | NO DATA |
| pseudomallei | | | | |
| Neisseria | FA 1090, ATCC 700825 | NO DATA | NO DATA | NO DATA |
| gonorrhoeae | | | | |
| Neisseria | MC58 (serogroup B) | NO DATA | NO DATA | NO DATA |
| meningitidis | | | | |
| Neisseria | serogroup C, FAM18 | NO DATA | NO DATA | NO DATA |
| meningitidis | | | | |
| Neisseria | Z2491 (serogroup A) | NO DATA | NO DATA | NO DATA |
| meningitidis | | | | |
| Chlamydophila | TW-183 | NO DATA | NO DATA | NO DATA |
| pneumoniae | | | | |
| Chlamydophila | AR39 | NO DATA | NO DATA | NO DATA |
| pneumoniae | | | | |
| Chlamydophila | CWL029 | NO DATA | NO DATA | NO DATA |
| pneumoniae | | | | |
| Chlamydophila | J138 | NO DATA | NO DATA | NO DATA |
| pneumoniae | | | | |
| Corynebacterium | NCTC13129 | NO DATA | NO DATA | NO DATA |
| diphtheriae | | | | |
| Mycobacterium | k10 | NO DATA | NO DATA | NO DATA |
| avium | | | | |
| Mycobacterium | 104 | NO DATA | NO DATA | NO DATA |
| avium | | | | |
| Mycobacterium | CSU#93 | NO DATA | NO DATA | NO DATA |
| tuberculosis | | | | |
| Mycobacterium | CDC 1551 | NO DATA | NO DATA | NO DATA |
| tuberculosis | | | | |
| Mycobacterium | H37Rv (lab strain) | NO DATA | NO DATA | NO DATA |
| tuberculosis | | | | |
| Mycoplasma | M129 | NO DATA | NO DATA | NO DATA |
| pneumoniae | | | | |
| Staphylococcus | MRSA252 | NO DATA | NO DATA | NO DATA |
| aureus | | | | |
| Staphylococcus | MSSA476 | NO DATA | NO DATA | NO DATA |
| aureus | | | | |
| Staphylococcus | COL | NO DATA | NO DATA | NO DATA |
| aureus | | | | |
| Staphylococcus | Mu50 | NO DATA | NO DATA | NO DATA |
| aureus | | | | |
| Staphylococcus | MW2 | NO DATA | NO DATA | NO DATA |
| aureus | | | | |
| Staphylococcus | N315 | NO DATA | NO DATA | NO DATA |
| aureus | | | | |
| Staphylococcus | NCTC 8325 | NO DATA | NO DATA | NO DATA |
| aureus | | | | |
| Streptococcus | NEM316 | NO DATA | NO DATA | NO DATA |
| agalactiae | | | | |
| Streptococcus | NC 002955 | NO DATA | NO DATA | NO DATA |
| equi | | | | |
| Streptococcus | MGAS8232 | NO DATA | NO DATA | NO DATA |
| pyogenes | | | | |
| Streptococcus | MGAS315 | NO DATA | NO DATA | NO DATA |
| pyogenes | | | | |
| Streptococcus | SSI-1 | NO DATA | NO DATA | NO DATA |
| pyogenes | | | | |
| Streptococcus | MGAS10394 | NO DATA | NO DATA | NO DATA |
| pyogenes | | | | |
| Streptococcus | Manfredo (M5) | NO DATA | NO DATA | NO DATA |
| pyogenes | | | | |
| Streptococcus | SF370 (M1) | NO DATA | NO DATA | NO DATA |
| pyogenes | | | | |
| Streptococcus | 670 | NO DATA | NO DATA | NO DATA |
| pneumoniae | | | | |
| Streptococcus | R6 | NO DATA | NO DATA | NO DATA |
| pneumoniae | | | | |
| Streptococcus | TIGR4 | NO DATA | NO DATA | NO DATA |
| pneumoniae | | | | |
| Streptococcus | NCTC7868 | NO DATA | NO DATA | NO DATA |
| gordonii | | | | |
| Streptococcus | NCTC 12261 | NO DATA | NO DATA | NO DATA |
| mitis | | | | |
| Streptococcus | UA159 | NO DATA | NO DATA | NO DATA |
| mutans | | | | |

TABLE 7E
Base Compositions of Common Respiratory Pathogens for Bioagent Identifying
Amplicons Corresponding to Primer Pair Nos: 362, 363, and 367

| | | Primer 362 | Primer 363 | Primer 367 |
|---|---|---|---|---|
| Organism | Strain | [A G C T] | [A G C T] | [A G C T] |
| Klebsiella | MGH78578 | [21 33 22 16] | [16 34 26 26] | NO DATA |
| pneumoniae | | | | |
| Yersinia pestis | CO-92 Biovar | [20 34 18 20] | NO DATA | NO DATA |
| | Orientalis | | | |
| Yersinia pestis | KIM5 P12 (Biovar | [20 34 18 20] | NO DATA | NO DATA |
| | Mediaevalis) | | | |
| Yersinia pestis | 91001 | [20 34 18 20] | NO DATA | NO DATA |
| Haemophilus | KW20 | NO DATA | NO DATA | NO DATA |
| influenzae | | | | |
| Pseudomonas | PAO1 | [19 35 21 17] | [16 36 28 22] | NO DATA |
| aeruginosa | | | | |
| Pseudomonas | Pf0-1 | NO DATA | [18 35 26 23] | NO DATA |
| fluorescens | | | | |
| Pseudomonas | KT2440 | NO DATA | [16 35 28 23] | NO DATA |
| putida | | | | |
| Legionella | Philadelphia-1 | NO DATA | NO DATA | NO DATA |
| pneumophila | | | | |
| Francisella | schu 4 | NO DATA | NO DATA | NO DATA |
| tularensis | | | | |
| Bordetella | Tohama I | [20 31 24 17] | [15 34 32 21] | [26 25 34 19] |
| pertussis | | | | |
| Burkholderia | J2315 | [20 33 21 18] | [15 36 26 25] | [25 27 32 20] |
| cepacia | | | | |
| Burkholderia | K96243 | [19 34 19 20] | [15 37 28 22] | [25 27 32 20] |
| pseudomallei | | | | |
| Neisseria | FA 1090, ATCC 700825 | NO DATA | NO DATA | NO DATA |
| gonorrhoeae | | | | |
| Neisseria | MC58 (serogroup B) | NO DATA | NO DATA | NO DATA |
| meningitidis | | | | |
| Neisseria | serogroup C, FAM18 | NO DATA | NO DATA | NO DATA |
| meningitidis | | | | |
| Neisseria | Z2491 (serogroup A) | NO DATA | NO DATA | NO DATA |
| meningitidis | | | | |
| Chlamydophila | TW-183 | NO DATA | NO DATA | NO DATA |
| pneumoniae | | | | |
| Chlamydophila | AR39 | NO DATA | NO DATA | NO DATA |
| pneumoniae | | | | |
| Chlamydophila | CWL029 | NO DATA | NO DATA | NO DATA |
| pneumoniae | | | | |
| Chlamydophila | J138 | NO DATA | NO DATA | NO DATA |
| pneumoniae | | | | |
| Corynebacterium | NCTC13129 | NO DATA | NO DATA | NO DATA |
| diphtheriae | | | | |
| Mycobacterium | k10 | [19 34 23 16] | NO DATA | [24 26 35 19] |
| avium | | | | |
| Mycobacterium | 104 | [19 34 23 16] | NO DATA | [24 26 35 19] |
| avium | | | | |
| Mycobacterium | CSU#93 | [19 31 25 17] | NO DATA | [25 25 34 20] |
| tuberculosis | | | | |
| Mycobacterium | CDC 1551 | [19 31 24 18] | NO DATA | [25 25 34 20] |
| tuberculosis | | | | |
| Mycobacterium | H37Rv (lab strain) | [19 31 24 18] | NO DATA | [25 25 34 20] |
| tuberculosis | | | | |
| Mycoplasma | M129 | NO DATA | NO DATA | NO DATA |
| pneumoniae | | | | |
| Staphylococcus | MRSA252 | NO DATA | NO DATA | NO DATA |
| aureus | | | | |
| Staphylococcus | MSSA476 | NO DATA | NO DATA | NO DATA |
| aureus | | | | |
| Staphylococcus | COL | NO DATA | NO DATA | NO DATA |
| aureus | | | | |
| Staphylococcus | Mu50 | NO DATA | NO DATA | NO DATA |
| aureus | | | | |
| Staphylococcus | MW2 | NO DATA | NO DATA | NO DATA |
| aureus | | | | |
| Staphylococcus | N315 | NO DATA | NO DATA | NO DATA |
| aureus | | | | |
| Staphylococcus | NCTC 8325 | NO DATA | NO DATA | NO DATA |
| aureus | | | | |
| Streptococcus | NEM316 | NO DATA | NO DATA | NO DATA |
| agalactiae | | | | |
| Streptococcus | NC_002955 | NO DATA | NO DATA | NO DATA |
| equi | | | | |
| Streptococcus | MGAS8232 | NO DATA | NO DATA | NO DATA |
| pyogenes | | | | |
| Streptococcus | MGAS315 | NO DATA | NO DATA | NO DATA |
| pyogenes | | | | |
| Streptococcus | SSI-1 | NO DATA | NO DATA | NO DATA |
| pyogenes | | | | |
| Streptococcus | MGAS10394 | NO DATA | NO DATA | NO DATA |
| pyogenes | | | | |
| Streptococcus | Manfredo (M5) | NO DATA | NO DATA | NO DATA |
| pyogenes | | | | |
| Streptococcus | SF370 (M1) | NO DATA | NO DATA | NO DATA |
| pyogenes | | | | |
| Streptococcus | 670 | NO DATA | NO DATA | NO DATA |
| pneumoniae | | | | |
| Streptococcus | R6 | [20 30 19 23] | NO DATA | NO DATA |
| pneumoniae | | | | |
| Streptococcus | TIGR4 | [20 30 19 23] | NO DATA | NO DATA |
| pneumoniae | | | | |
| Streptococcus | NCTC7868 | NO DATA | NO DATA | NO DATA |
| gordonii | | | | |
| Streptococcus | NCTC 12261 | NO DATA | NO DATA | NO DATA |
| mitis | | | | |
| Streptococcus | UA159 | NO DATA | NO DATA | NO DATA |
| mutans | | | | |

Four sets of throat samples from military recruits at different military facilities taken at different time points were analyzed using the primers of the present invention. The first set was collected at a military training center from Nov. 1 to Dec. 20, 2002 during one of the most severe outbreaks of pneumonia associated with group A Streptococcus in the United States since 1968. During this outbreak, fifty-one throat swabs were taken from both healthy and hospitalized recruits and plated on blood agar for selection of putative group A Streptococcus colonies. A second set of 15 original patient specimens was taken during the height of this group A Streptococcus-associated respiratory disease outbreak. The third set were historical samples, including twenty-seven isolates of group A Streptococcus, from disease outbreaks at this and other military training facilities during previous years. The fourth set of samples was collected from five geographically separated military facilities in the continental U.S. in the winter immediately following the severe November/December 2002 outbreak.

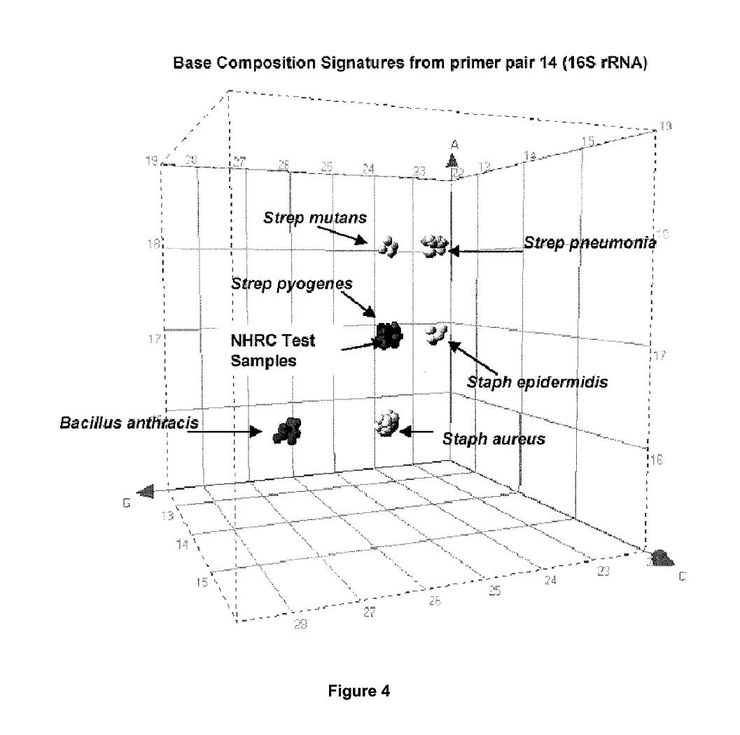

Pure colonies isolated from group A Streptococcus-selective media from all four collection periods were analyzed with the surveillance primer set. All samples showed base compositions that precisely matched the four completely sequenced strains of Streptococcus pyogenes. Shown in FIG. 4 is a 3D diagram of base composition (axes A, G and C) of bioagent identifying amplicons obtained with primer pair number 14 (a precursor of primer pair number 348 which targets 16S rRNA). The diagram indicates that the experimentally determined base compositions of the clinical samples closely match the base compositions expected for Streptococcus pyogenes and are distinct from the expected base compositions of other organisms.

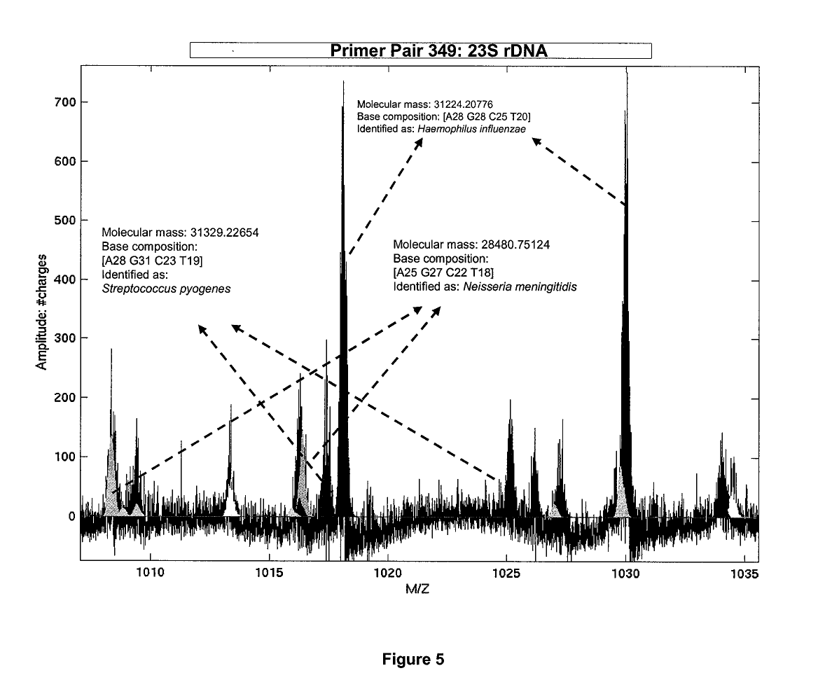

In addition to the identification of Streptococcus pyogenes, other potentially pathogenic organisms were identified concurrently. Mass spectral analysis of a sample whose nucleic acid was amplified by primer pair number 349 (SEQ ID NOs: 401:1156) exhibited signals of bioagent identifying amplicons with molecular masses that were found to correspond to analogous base compositions of bioagent identifying amplicons of Streptococcus pyogenes (A27 G32 C24 T18), Neisseria meningitidis (A25 G27 C22 T18), and Haemophilus influenzae (A28 G28 C25 T20) (see FIG. 5 and Table 7B). These organisms were present in a ratio of 4:5:20 as determined by comparison of peak heights with peak height of an internal PCR calibration standard as described in commonly owned U.S. Patent Application Ser. No: 60/545,425 which is incorporated herein by reference in its entirety.

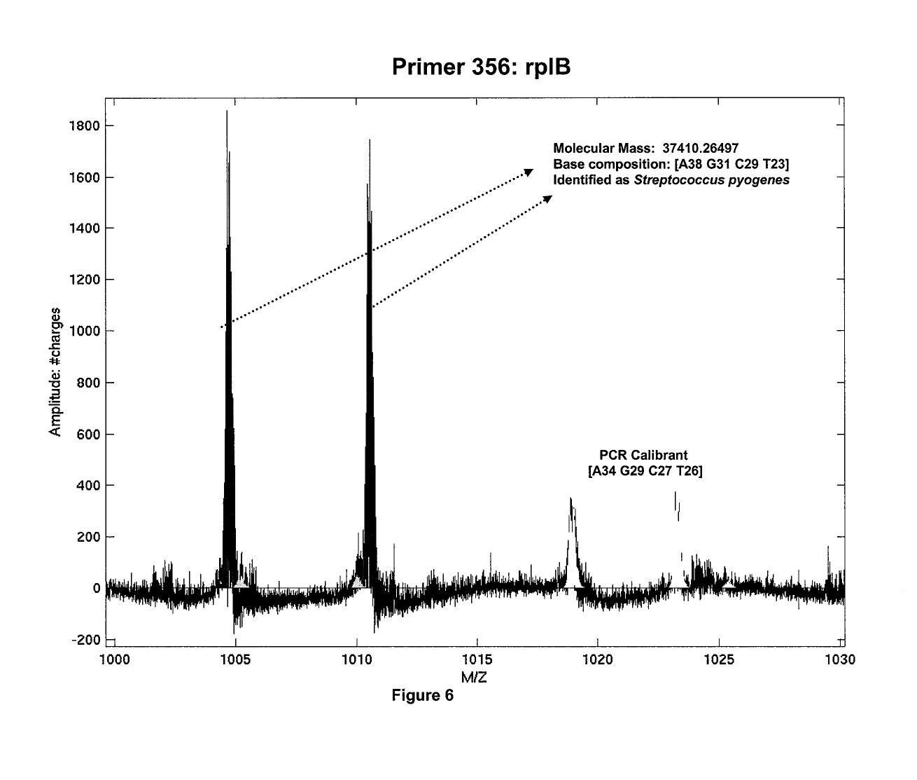

Since certain division-wide primers that target housekeeping genes are configured to provide coverage of specific divisions of bacteria to increase the confidence level for identification of bacterial species, they are not expected to yield bioagent identifying amplicons for organisms outside of the specific divisions. For example, primer pair number 356 (SEQ ID NOs: 449:1380) primarily amplifies the nucleic acid of members of the classes Bacilli and Clostridia and is not expected to amplify proteobacteria such as Neisseria meningitidis and Haemophilus influenzae. As expected, analysis of the mass spectrum of amplification products obtained with primer pair number 356 does not indicate the presence of Neisseria meningitidis and Haemophilus influenzae but does indicate the presence of Streptococcus pyogenes (FIGS. 3 and 6, Table 7B). Thus, these primers or types of primers can confirm the absence of particular bioagents from a sample.

The 15 throat swabs from military recruits were found to contain a relatively small set of microbes in high abundance. The most common were Haemophilus influenza, Neisseria meningitides, and Streptococcus pyogenes. Staphylococcus epidermidis, Moraxella cattarhalis, Corynebacterium pseudodiphtheriticum, and Staphylococcus aureus were present in fewer samples. An equal number of samples from healthy volunteers from three different geographic locations, were identically analyzed. Results indicated that the healthy volunteers have bacterial flora dominated by multiple, commensal non-beta-hemolytic Streptococcal species, including the viridans group streptococci (S. parasangunis, S. vestibularis, S. mitis, S. oralis and S. pneumoniae; data not shown), and none of the organisms found in the military recruits were found in the healthy controls at concentrations detectable by mass spectrometry. Thus, the military recruits in the midst of a respiratory disease outbreak had a dramatically different microbial population than that experienced by the general population in the absence of epidemic disease.

### Example 7

### Triangulation Genotyping Analysis for Determination of emm-Type of Streptococcus pyogenes in Epidemic Surveillance

As a continuation of the epidemic surveillance investigation of Example 6, determination of sub-species characteristics (genotyping) of Streptococcus pyogenes, was carried out based on a strategy that generates strain-specific signatures according to the rationale of Multi-Locus Sequence Typing (MLST). In classic MLST analysis, internal fragments of several housekeeping genes are amplified and sequenced (Enright et al. Infection and Immunity, 2001, 69, 2416-2427). In classic MLST analysis, internal fragments of several housekeeping genes are amplified and sequenced. In the present investigation, bioagent identifying amplicons from housekeeping genes were produced using drill-down primers and analyzed by mass spectrometry. Since mass spectral analysis results in molecular mass, from which base composition can be determined, the challenge was to determine whether resolution of emm classification of strains of Streptococcus pyogenes could be determined.

For the purpose of development of a triangulation genotyping assay, an alignment was constructed of concatenated alleles of seven MLST housekeeping genes (glucose kinase (gki), glutamine transporter protein (gtr), glutamate racemase (murl), DNA mismatch repair protein (mutS), xanthine phosphoribosyl transferase (xpt), and acetyl-CoA acetyl transferase (yqiL)) from each of the 212 previously emm-typed strains of Streptococcus pyogenes. From this alignment, the number and location of primer pairs that would maximize strain identification via base composition was determined. As a result, 6 primer pairs were chosen as standard drill-down primers for determination of emm-type of Streptococcus pyogenes. These six primer pairs are displayed in Table 8. This drill-down set comprises primers with T modifications (note TMOD designation in primer names) which constitutes a functional improvement with regard to prevention of non-templated adenylation (vide supra) relative to originally selected primers which are displayed below in the same row.

TABLE 8
Triangulation Genotyping Analysis Primer Pairs for Group A Streptococcus Drill-Down

| | | Forward | | Reverse | |
|---|---|---|---|---|---|
| | | Primer | | Primer | |
| Primer | | (SEQ | | (SEQ | Target |
| Pair No. | Forward Primer Name | ID NO:) | Reverse Primer Name | ID NO:) | Gene |
| 442 | SP101_SPET11_358_387_TMOD_F | 588 | SP101_SPET11_448_473_TMOD_R | 998 | gki |
| 80 | SP101_SPET11_358_387_F | 126 | SP101_SPET11_448_473_TMOD_R | 766 | gki |
| 443 | SP101_SPET11_600_629_TMOD_F | 348 | SP101_SPET11_686_714_TMOD_R | 1018 | gtr |
| 81 | SP101_SPET11_600_629_F | 62 | SP101_SPET11_686_714_R | 772 | gtr |
| 426 | SP101_SPET11_1314_1336_TMOD_F | 363 | SP101_SPET11_1403_1431_TMOD_R | 849 | murI |
| 86 | SP101_SPET11_1314_1336_F | 68 | SP101_SPET11_1403_1431_R | 711 | murI |
| 430 | SP101_SPET11_1807_1835_TMOD_F | 235 | SP101_SPET11_1901_1927_TMOD_R | 1439 | mutS |
| 90 | SP101_SPET11_1807_1835_F | 33 | SP101_SPET11_1901_1927_R | 1412 | mutS |
| 438 | SP101_SPET11_3075_3103_TMOD_F | 473 | SP101_SPET11_3168_3196_TMOD_R | 875 | xpt |
| 96 | SP101_SPET11_3075_3103_F | 108 | SP101_SPET11_3168_3196_R | 715 | xpt |
| 441 | SP101_SPET11_3511_3535_TMOD_F | 531 | SP101_SPET11_3605_3629_TMOD_R | 1294 | yqiL |
| 98 | SP101_SPET11_3511_3535_F | 116 | SP101_SPET11_3605_3629_R | 832 | yqiL |

The primers of Table 8 were used to produce bioagent identifying amplicons from nucleic acid present in the clinical samples. The bioagent identifying amplicons which were subsequently analyzed by mass spectrometry and base compositions corresponding to the molecular masses were calculated.

Of the 51 samples taken during the peak of the November/December 2002 epidemic (Table 9A-C rows 1-3), all except three samples were found to represent emm3, a Group A Streptococcus genotype previously associated with high respiratory virulence. The three outliers were from samples obtained from healthy individuals and probably represent non-epidemic strains. Archived samples (Tables 9A-C rows 5-13) from historical collections showed a greater heterogeneity of base compositions and emm types as would be expected from different epidemics occurring at different places and dates. The results of the mass spectrometry analysis and emm gene sequencing were found to be concordant for the epidemic and historical samples.

TABLE 9A
Base Composition Analysis of Bioagent Identifying Amplicons of Group A
Streptococcus samples from Six Military Installations Obtained with Primer Pair Nos. 426 and 430

| | emm-type by | | | | murI | mutS |
|---|---|---|---|---|---|---|
| # of | Mass | emm-Gene | Location | | (Primer Pair | (Primer Pair |
| Instances | Spectrometry | Sequencing | (sample) | Year | No. 426) | No. 430) |
| 48 | 3 | 3 | MCRD San | 2002 | A39 G25 C20 T34 | A38 G27 C23 T33 |
| 2 | 6 | 6 | Diego | | A40 G24 C20 T34 | A38 G27 C23 T33 |
| 1 | 28 | 28 | (Cultured) | | A39 G25 C20 T34 | A38 G27 C23 T33 |
| 15 | 3 | ND | | | A39 G25 C20 T34 | A38 G27 C23 T33 |
| 6 | 3 | 3 | NHRC San | 2003 | A39 G25 C20 T34 | A38 G27 C23 T33 |
| 3 | 5, 58 | 5 | Diego- | | A40 G24 C20 T34 | A38 G27 C23 T33 |
| 6 | 6 | 6 | Archive | | A40 G24 C20 T34 | A38 G27 C23 T33 |
| 1 | 11 | 11 | (Cultured) | | A39 G25 C20 T34 | A38 G27 C23 T33 |
| 3 | 12 | 12 | | | A40 G24 C20 T34 | A38 G26 C24 T33 |
| 1 | 22 | 22 | | | A39 G25 C20 T34 | A38 G27 C23 T33 |
| 3 | 25, 75 | 75 | | | A39 G25 C20 T34 | A38 G27 C23 T33 |
| 4 | 44/61, 82, 9 | 44/61 | | | A40 G24 C20 T34 | A38 G26 C24 T33 |
| 2 | 53, 91 | 91 | | | A39 G25 C20 T34 | A38 G27 C23 T33 |
| 1 | 2 | 2 | Ft. | 2003 | A39 G25 C20 T34 | A38 G27 C24 T32 |
| 2 | 3 | 3 | Leonard | | A39 G25 C20 T34 | A38 G27 C23 T33 |
| 1 | 4 | 4 | Wood | | A39 G25 C20 T34 | A38 G27 C23 T33 |
| 1 | 6 | 6 | (Cultured) | | A40 G24 C20 T34 | A38 G27 C23 T33 |
| 11 | 25 or 75 | 75 | | | A39 G25 C20 T34 | A38 G27 C23 T33 |
| 1 | 25, 75, 33, | 75 | | | A39 G25 C20 T34 | A38 G27 C23 T33 |
| | 34, 4, 52, 84 | | | | | |
| 1 | 44/61 or 82 | 44/61 | | | A40 G24 C20 T34 | A38 G26 C24 T33 |
| | or 9 | | | | | |
| 2 | 5 or 58 | 5 | | | A40 G24 C20 T34 | A38 G27 C23 T33 |
| 3 | 1 | 1 | Ft. Sill | 2003 | A40 G24 C20 T34 | A38 G27 C23 T33 |
| 2 | 3 | 3 | (Cultured) | | A39 G25 C20 T34 | A38 G27 C23 T33 |
| 1 | 4 | 4 | | | A39 G25 C20 T34 | A38 G27 C23 T33 |
| 1 | 28 | 28 | | | A39 G25 C20 T34 | A38 G27 C23 T33 |
| 1 | 3 | 3 | Ft. | 2003 | A39 G25 C20 T34 | A38 G27 C23 T33 |
| 1 | 4 | 4 | Benning | | A39 G25 C20 T34 | A38 G27 C23 T33 |
| 3 | 6 | 6 | (Cultured) | | A40 G24 C20 T34 | A38 G27 C23 T33 |
| 1 | 11 | 11 | | | A39 G25 C20 T34 | A38 G27 C23 T33 |
| 1 | 13 | 94** | | | A40 G24 C20 T34 | A38 G27 C23 T33 |
| 1 | 44/61 or 82 | 82 | | | A40 G24 C20 T34 | A38 G26 C24 T33 |
| | or 9 | | | | | |
| 1 | 5 or 58 | 58 | | | A40 G24 C20 T34 | A38 G27 C23 T33 |
| 1 | 78 or 89 | 89 | | | A39 G25 C20 T34 | A38 G27 C23 T33 |
| 2 | 5 or 58 | ND | Lackland | 2003 | A40 G24 C20 T34 | A38 G27 C23 T33 |
| 1 | 2 | | AFB | | A39 G25 C20 T34 | A38 G27 C24 T32 |
| 1 | 81 or 90 | | (Throat | | A40 G24 C20 T34 | A38 G27 C23 T33 |
| 1 | 78 | | Swabs) | | A38 G26 C20 T34 | A38 G27 C23 T33 |
| 3*** | No detection | | | | No detection | No detection |
| 7 | 3 | ND | MCRD San | 2002 | A39 G25 C20 T34 | A38 G27 C23 T33 |
| 1 | 3 | ND | Diego | | No detection | A38 G27 C23 T33 |
| 1 | 3 | ND | (Throat | | No detection | No detection |
| 1 | 3 | ND | Swabs) | | No detection | No detection |
| 2 | 3 | ND | | | No detection | A38 G27 C23 T33 |
| 3 | No detection | ND | | | No detection | No detection |

TABLE 9B
Base Composition Analysis of Bioagent Identifying Amplicons of Group A
Streptococcus samples from Six Military Installations Obtained with Primer Pair Nos. 438 and 441

| | emm-type by | | | | xpt | yqiL |
|---|---|---|---|---|---|---|
| # of | Mass | emm-Gene | Location | | (Primer Pair | (Primer Pair |
| Instances | Spectrometry | Sequencing | (sample) | Year | No. 438) | No. 441) |
| 48 | 3 | 3 | MCRD San | 2002 | A30 G36 C20 T36 | A40 G29 C19 T31 |
| 2 | 6 | 6 | Diego | | A30 G36 C20 T36 | A40 G29 C19 T31 |
| 1 | 28 | 28 | (Cultured) | | A30 G36 C20 T36 | A41 G28 C18 T32 |
| 15 | 3 | ND | | | A30 G36 C20 T36 | A40 G29 C19 T31 |
| 6 | 3 | 3 | NHRC San | 2003 | A30 G36 C20 T36 | A40 G29 C19 T31 |
| 3 | 5, 58 | 5 | Diego- | | A30 G36 C20 T36 | A40 G29 C19 T31 |
| 6 | 6 | 6 | Archive | | A30 G36 C20 T36 | A40 G29 C19 T31 |
| 1 | 11 | 11 | (Cultured) | | A30 G36 C20 T36 | A40 G29 C19 T31 |
| 3 | 12 | 12 | | | A30 G36 C19 T37 | A40 G29 C19 T31 |
| 1 | 22 | 22 | | | A30 G36 C20 T36 | A40 G29 C19 T31 |
| 3 | 25, 75 | 75 | | | A30 G36 C20 T36 | A40 G29 C19 T31 |
| 4 | 44/61, 82, 9 | 44/61 | | | A30 G36 C20 T36 | A41 G28 C19 T31 |
| 2 | 53, 91 | 91 | | | A30 G36 C19 T37 | A40 G29 C19 T31 |
| 1 | 2 | 2 | Ft. | 2003 | A30 G36 C20 T36 | A40 G29 C19 T31 |
| 2 | 3 | 3 | Leonard | | A30 G36 C20 T36 | A40 G29 C19 T31 |
| 1 | 4 | 4 | Wood | | A30 G36 C19 T37 | A41 G28 C19 T31 |
| 1 | 6 | 6 | (Cultured) | | A30 G36 C20 T36 | A40 G29 C19 T31 |
| 11 | 25 or 75 | 75 | | | A30 G36 C20 T36 | A40 G29 C19 T31 |
| 1 | 25, 75, 33, | 75 | | | A30 G36 C19 T37 | A40 G29 C19 T31 |
| | 34, 4, 52, 84 | | | | | |
| 1 | 44/61 or 82 | 44/61 | | | A30 G36 C20 T36 | A41 G28 C19 T31 |
| | or 9 | | | | | |
| 2 | 5 or 58 | 5 | | | A30 G36 C20 T36 | A40 G29 C19 T31 |
| 3 | 1 | 1 | Ft. Sill | 2003 | A30 G36 C19 T37 | A40 G29 C19 T31 |
| 2 | 3 | 3 | (Cultured) | | A30 G36 C20 T36 | A40 G29 C19 T31 |
| 1 | 4 | 4 | | | A30 G36 C19 T37 | A41 G28 C19 T31 |
| 1 | 28 | 28 | | | A30 G36 C20 T36 | A41 G28 C18 T32 |
| 1 | 3 | 3 | Ft. | 2003 | A30 G36 C20 T36 | A40 G29 C19 T31 |
| 1 | 4 | 4 | Benning | | A30 G36 C19 T37 | A41 G28 C19 T31 |
| 3 | 6 | 6 | (Cultured) | | A30 G36 C20 T36 | A40 G29 C19 T31 |
| 1 | 11 | 11 | | | A30 G36 C20 T36 | A40 G29 C19 T31 |
| 1 | 13 | 94** | | | A30 G36 C20 T36 | A41 G28 C19 T31 |
| 1 | 44/61 or 82 | 82 | | | A30 G36 C20 T36 | A41 G28 C19 T31 |
| | or 9 | | | | | |
| 1 | 5 or 58 | 58 | | | A30 G36 C20 T36 | A40 G29 C19 T31 |
| 1 | 78 or 89 | 89 | | | A30 G36 C20 T36 | A41 G28 C19 T31 |
| 2 | 5 or 58 | ND | Lackland | 2003 | A30 G36 C20 T36 | A40 G29 C19 T31 |
| 1 | 2 | | AFB | | A30 G36 C20 T36 | A40 G29 C19 T31 |
| 1 | 81 or 90 | | (Throat | | A30 G36 C20 T36 | A40 G29 C19 T31 |
| 1 | 78 | | Swabs) | | A30 G36 C20 T36 | A41 G28 C19 T31 |
| 3*** | No detection | | | | No detection | No detection |
| 7 | 3 | ND | MCRD San | 2002 | A30 G36 C20 T36 | A40 G29 C19 T31 |
| 1 | 3 | ND | Diego | | A30 G36 C20 T36 | A40 G29 C19 T31 |
| 1 | 3 | ND | (Throat | | A30 G36 C20 T36 | No detection |
| 1 | 3 | ND | Swabs) | | No detection | A40 G29 C19 T31 |
| 2 | 3 | ND | | | A30 G36 C20 T36 | A40 G29 C19 T31 |
| 3 | No detection | ND | | | No detection | No detection |

TABLE 9C
Base Composition Analysis of Bioagent Identifying Amplicons of Group A
Streptococcus samples from Six Military Installations Obtained with Primer Pair Nos. 438 and 441

| | emm-type by | | | | gki | gtr |
|---|---|---|---|---|---|---|
| # of | Mass | emm-Gene | Location | | (Primer Pair | ((Primer Pair |
| Instances | Spectrometry | Sequencing | (sample) | Year | No. 442) | No. 443) |
| 48 | 3 | 3 | MCRD San | 2002 | A32 G35 C17 T32 | A39 G28 C16 T32 |
| 2 | 6 | 6 | Diego | | A31 G35 C17 T33 | A39 G28 C15 T33 |
| 1 | 28 | 28 | (Cultured) | | A30 G36 C17 T33 | A39 G28 C16 T32 |
| 15 | 3 | ND | | | A32 G35 C17 T32 | A39 G28 C16 T32 |
| 6 | 3 | 3 | NHRC San | 2003 | A32 G35 C17 T32 | A39 G28 C16 T32 |
| 3 | 5, 58 | 5 | Diego- | | A30 G36 C20 T30 | A39 G28 C15 T33 |
| 6 | 6 | 6 | Archive | | A31 G35 C17 T33 | A39 G28 C15 T33 |
| 1 | 11 | 11 | (Cultured) | | A30 G36 C20 T30 | A39 G28 C16 T32 |
| 3 | 12 | 12 | | | A31 G35 C17 T33 | A39 G28 C15 T33 |
| 1 | 22 | 22 | | | A31 G35 C17 T33 | A38 G29 C15 T33 |
| 3 | 25, 75 | 75 | | | A30 G36 C17 T33 | A39 G28 C15 T33 |
| 4 | 44/61, 82, 9 | 44/61 | | | A30 G36 C18 T32 | A39 G28 C15 T33 |
| 2 | 53, 91 | 91 | | | A32 G35 C17 T32 | A39 G28 C16 T32 |
| 1 | 2 | 2 | Ft. | 2003 | A30 G36 C17 T33 | A39 G28 C15 T33 |
| 2 | 3 | 3 | Leonard | | A32 G35 C17 T32 | A39 G28 C16 T32 |
| 1 | 4 | 4 | Wood | | A31 G35 C17 T33 | A39 G28 C15 T33 |
| 1 | 6 | 6 | (Cultured) | | A31 G35 C17 T33 | A39 G28 C15 T33 |
| 11 | 25 or 75 | 75 | | | A30 G36 C17 T33 | A39 G28 C15 T33 |
| 1 | 25, 75, 33, | 75 | | | A30 G36 C17 T33 | A39 G28 C15 T33 |
| | 34, 4, 52, 84 | | | | | |
| 1 | 44/61 or 82 | 44/61 | | | A30 G36 C18 T32 | A39 G28 C15 T33 |
| | or 9 | | | | | |
| 2 | 5 or 58 | 5 | | | A30 G36 C20 T30 | A39 G28 C15 T33 |
| 3 | 1 | 1 | Ft. Sill | 2003 | A30 G36 C18 T32 | A39 G28 C15 T33 |
| 2 | 3 | 3 | (Cultured) | | A32 G35 C17 T32 | A39 G28 C16 T32 |
| 1 | 4 | 4 | | | A31 G35 C17 T33 | A39 G28 C15 T33 |
| 1 | 28 | 28 | | | A30 G36 C17 T33 | A39 G28 C16 T32 |
| 1 | 3 | 3 | Ft. | 2003 | A32 G35 C17 T32 | A39 G28 C16 T32 |
| 1 | 4 | 4 | Benning | | A31 G35 C17 T33 | A39 G28 C15 T33 |
| 3 | 6 | 6 | (Cultured) | | A31 G35 C17 T33 | A39 G28 C15 T33 |
| 1 | 11 | 11 | | | A30 G36 C20 T30 | A39 G28 C16 T32 |
| 1 | 13 | 94** | | | A30 G36 C19 T31 | A39 G28 C15 T33 |
| 1 | 44/61 or 82 | 82 | | | A30 G36 C18 T32 | A39 G28 C15 T33 |
| | or 9 | | | | | |
| 1 | 5 or 58 | 58 | | | A30 G36 C20 T30 | A39 G28 C15 T33 |
| 1 | 78 or 89 | 89 | | | A30 G36 C18 T32 | A39 G28 C15 T33 |
| 2 | 5 or 58 | ND | Lackland | 2003 | A30 G36 C20 T30 | A39 G28 C15 T33 |
| 1 | 2 | | AFB | | A30 G36 C17 T33 | A39 G28 C15 T33 |
| 1 | 81 or 90 | | (Throat | | A30 G36 C17 T33 | A39 G28 C15 T33 |
| 1 | 78 | | Swabs) | | A30 G36 C18 T32 | A39 G28 C15 T33 |
| 3*** | No detection | | | | No detection | No detection |
| 7 | 3 | ND | MCRD San | 2002 | A32 G35 C17 T32 | A39 G28 C16 T32 |
| 1 | 3 | ND | Diego | | No detection | No detection |
| 1 | 3 | ND | (Throat | | A32 G35 C17 T32 | A39 G28 C16 T32 |
| 1 | 3 | ND | Swabs) | | A32 G35 C17 T32 | No detection |
| 2 | 3 | ND | | | A32 G35 C17 T32 | No detection |
| 3 | No detection | ND | | | No detection | No detection |

### Example 8

### Design of Calibrant Polynucleotides Based on Bioagent Identifying Amplicons for Identification of Species of Bacteria (Bacterial Bioagent Identifying Amplicons)

This example describes the design of 19 calibrant polynucleotides based on bacterial bioagent identifying amplicons corresponding to the primers of the broad surveillance set (Table 5) and the Bacillus anthracis drill-down set (Table 6).

Calibration sequences were designed to simulate bacterial bioagent identifying amplicons produced by the T modified primer pairs shown in Tables 5 and 6 (primer names have the designation “TMOD”). The calibration sequences were chosen as a representative member of the section of bacterial genome from specific bacterial species which would be amplified by a given primer pair. The model bacterial species upon which the calibration sequences are based are also shown in Table 10. For example, the calibration sequence chosen to correspond to an amplicon produced by primer pair no. 361 is SEQ ID NO: 1445. In Table 10, the forward (_F) or reverse (_R) primer name indicates the coordinates of an extraction representing a gene of a standard reference bacterial genome to which the primer hybridizes e.g.: the forward primer name 16S_EC_{—}713_{—}732_TMOD_F indicates that the forward primer hybridizes to residues 713-732 of the gene encoding 16S ribosomal RNA in an E. coli reference sequence (in this case, the reference sequence is an extraction consisting of residues 4033120-4034661 of the genomic sequence of E. coli K12 (GenBank gi number 16127994). Additional gene coordinate reference information is shown in Table 11. The designation “TMOD” in the primer names indicates that the 5′ end of the primer has been modified with a non-matched template T residue which prevents the PCR polymerase from adding non-templated adenosine residues to the 5′ end of the amplification product, an occurrence which may result in miscalculation of base composition from molecular mass data (vide supra).

The 19 calibration sequences described in Tables 10 and 11 were combined into a single calibration polynucleotide sequence (SEQ ID NO: 1464—which is herein designated a “combination calibration polynucleotide”) which was then cloned into a pCR®-Blunt vector (Invitrogen, Carlsbad, Calif.). This combination calibration polynucleotide can be used in conjunction with the primers of Tables 5 or 6 as an internal standard to produce calibration amplicons for use in determination of the quantity of any bacterial bioagent. Thus, for example, when the combination calibration polynucleotide vector is present in an amplification reaction mixture, a calibration amplicon based on primer pair 346 (16S rRNA) will be produced in an amplification reaction with primer pair 346 and a calibration amplicon based on primer pair 363 (rpoC) will be produced with primer pair 363. Coordinates of each of the 19 calibration sequences within the calibration polynucleotide (SEQ ID NO: 1464) are indicated in Table 11.

TABLE 10
Bacterial Primer Pairs for Production of Bacterial Bioagent Identifying
Amplicons and Corresponding Representative Calibration Sequences

| | | | | | Calibration | Calibration |
|---|---|---|---|---|---|---|
| Primer | | Forward | | Reverse | Sequence | Sequence |
| Pair | | Primer | | Primer | Model | (SEQ ID |
| No. | Forward Primer Name | (SEQ ID NO:) | Reverse Primer Name | (SEQ ID NO:) | Species | NO:) |
| 361 | 16S_EC_1090_1111_2_TMOD_F | 697 | 16S_EC_1175_1196_TMOD_R | 1398 | Bacillus | 1445 |
| | | | | | anthracis | |
| 346 | 16S_EC_713_732_TMOD_F | 202 | 16S_EC_789_809_TMOD_R | 1110 | Bacillus | 1446 |
| | | | | | anthracis | |
| 347 | 16S_EC_785_806_TMOD_F | 560 | 16S_EC_880_897_TMOD_R | 1278 | Bacillus | 1447 |
| | | | | | anthracis | |
| 348 | 16S_EC_960_981_TMOD_F | 706 | 16S_EC_1054_1073_TMOD_R | 895 | Bacillus | 1448 |
| | | | | | anthracis | |
| 349 | 23S_EC_1826_1843_TMOD_F | 401 | 23S_EC_1906_1924_TMOD_R | 1156 | Bacillus | 1449 |
| | | | | | anthracis | |
| 360 | 23S_EC_2646_2667_TMOD_F | 409 | 23S_EC_2745_2765_TMOD_R | 1434 | Bacillus | 1450 |
| | | | | | anthracis | |
| 350 | CAPC_BA_274_303_TMOD_F | 476 | CAPC_BA_349_376_TMOD_R | 1314 | Bacillus | 1451 |
| | | | | | anthracis | |
| 351 | CYA_BA_1353_1379_TMOD_F | 355 | CYA_BA_1448_1467_TMOD_R | 1423 | Bacillus | 1452 |
| | | | | | anthracis | |
| 352 | INFB_EC_1365_1393_TMOD_F | 687 | INFB_EC_1439_1467_TMOD_R | 1411 | Bacillus | 1453 |
| | | | | | anthracis | |
| 353 | LEF_BA_756_781_TMOD_F | 220 | LEF_BA_843_872_TMOD_R | 1394 | Bacillus | 1454 |
| | | | | | anthracis | |
| 356 | RPLB_EC_650_679_TMOD_F | 449 | RPLB_EC_739_762_TMOD_R | 1380 | Clostridium | 1455 |
| | | | | | botulinum | |
| 449 | RPLB_EC_690_710_F | 309 | RPLB_EC_737_758_R | 1336 | Clostridium | 1456 |
| | | | | | botulinum | |
| 359 | RPOB_EC_1845_1866_TMOD_F | 659 | RPOB_EC_1909_1929_TMOD_R | 1250 | Yersinia | 1457 |
| | | | | | Pestis | |
| 362 | RPOB_EC_3799_3821_TMOD_F | 581 | RPOB_EC_3862_3888_TMOD_R | 1325 | Burkholderia | 1458 |
| | | | | | mallei | |
| 363 | RPOC_EC_2146_2174_TMOD_F | 284 | RPOC_EC_2227_2245_TMOD_R | 898 | Burkholderia | 1459 |
| | | | | | mallei | |
| 354 | RPOC_EC_2218_2241_TMOD_F | 405 | RPOC_EC_2313_2337_TMOD_R | 1072 | Bacillus | 1460 |
| | | | | | anthracis | |
| 355 | SSPE_BA_115_137_TMOD_F | 255 | SSPE_BA_197_222_TMOD_R | 1402 | Bacillus | 1461 |
| | | | | | anthracis | |
| 367 | TUFB_EC_957_979_TMOD_F | 308 | TUFB_EC_1034_1058_TMOD_R | 1276 | Burkholderia | 1462 |
| | | | | | mallei | |
| 358 | VALS_EC_1105_1124_TMOD_F | 385 | VALS_EC_1195_1218_TMOD_R | 1093 | Yersinia | 1463 |
| | | | | | Pestis | |

TABLE 11
Primer Pair Gene Coordinate References and Calibration Polynucleotide Sequence
Coordinates within the Combination Calibration Polynucleotide

| | | | | Coordinates of |
|---|---|---|---|---|
| | | | | Calibration Sequence |
| | Gene Extraction | Reference GenBank | | in Combination |
| Bacterial | Coordinates | GI No. of Genomic | | Calibration |
| Gene and | of Genomic or Plasmid | (G) or Plasmid (P) | Primer | Polynucleotide |
| Species | Sequence | Sequence | Pair No. | (SEQ ID NO: 1464) |
| 16S E. coli | 4033120 . . . 4034661 | 16127994 (G) | 346 | 16 . . . 109 |
| 16S E. coli | 4033120 . . . 4034661 | 16127994 (G) | 347 | 83 . . . 190 |
| 16S E. coli | 4033120 . . . 4034661 | 16127994 (G) | 348 | 246 . . . 353 |
| 16S E. coli | 4033120 . . . 4034661 | 16127994 (G) | 361 | 368 . . . 469 |
| 23S E. coli | 4166220 . . . 4169123 | 16127994 (G) | 349 | 743 . . . 837 |
| 23S E. coli | 4166220 . . . 4169123 | 16127994 (G) | 360 | 865 . . . 981 |
| rpoB E. coli | 4178823 . . . 4182851 | 16127994 (G) | 359 | 1591 . . . 1672 |
| | (complement strand) | | | |
| rpoB E. coli | 4178823 . . . 4182851 | 16127994 (G) | 362 | 2081 . . . 2167 |
| | (complement strand) | | | |
| rpoC E. coli | 4182928 . . . 4187151 | 16127994 (G) | 354 | 1810 . . . 1926 |
| rpoC E. coli | 4182928 . . . 4187151 | 16127994 (G) | 363 | 2183 . . . 2279 |
| infB E. coli | 3313655 . . . 3310983 | 16127994 (G) | 352 | 1692 . . . 1791 |
| | (complement strand) | | | |
| tufB E. coli | 4173523 . . . 4174707 | 16127994 (G) | 367 | 2400 . . . 2498 |
| rplB E. coli | 3449001 . . . 3448180 | 16127994 (G) | 356 | 1945 . . . 2060 |
| rplB E. coli | 3449001 . . . 3448180 | 16127994 (G) | 449 | 1986 . . . 2055 |
| valS E. coli | 4481405 . . . 4478550 | 16127994 (G) | 358 | 1462 . . . 1572 |
| | (complement strand) | | | |
| capC | 56074 . . . 55628 | 6470151 (P) | 350 | 2517 . . . 2616 |
| B. anthracis | (complement strand) | | | |
| cya | 156626 . . . 154288 | 4894216 (P) | 351 | 1338 . . . 1449 |
| B. anthracis | (complement strand) | | | |
| lef | 127442 . . . 129921 | 4894216 (P) | 353 | 1121 . . . 1234 |
| B. anthracis | | | | |
| sspE | 226496 . . . 226783 | 30253828 (G) | 355 | 1007-1104 |
| B. anthracis | | | | |

### Example 9

### Use of a Calibration Polynucleotide for Determining the Quantity of Bacillus Anthracis in a Sample Containing a Mixture of Microbes

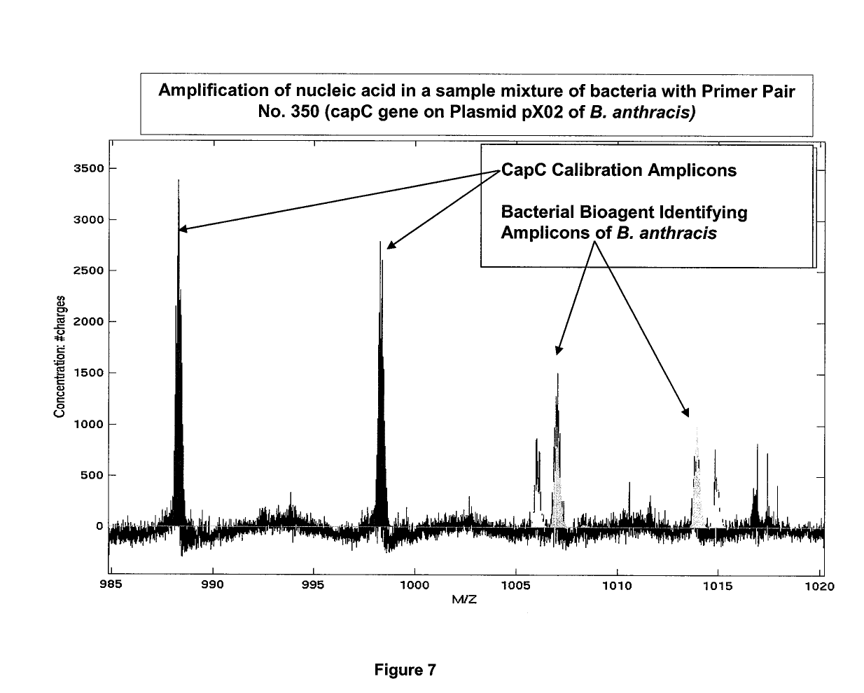

The process described in this example is shown in FIG. 2. The capC gene is a gene involved in capsule synthesis which resides on the pX02 plasmid of Bacillus anthracis. Primer pair number 350 (see Tables 10 and 11) was configured to identify Bacillus anthracis via production of a bacterial bioagent identifying amplicon. Known quantities of the combination calibration polynucleotide vector described in Example 8 were added to amplification mixtures containing bacterial bioagent nucleic acid from a mixture of microbes which included the Ames strain of Bacillus anthracis. Upon amplification of the bacterial bioagent nucleic acid and the combination calibration polynucleotide vector with primer pair no. 350, bacterial bioagent identifying amplicons and calibration amplicons were obtained and characterized by mass spectrometry. A mass spectrum measured for the amplification reaction is shown in FIG. 7. The molecular masses of the bioagent identifying amplicons provided the means for identification of the bioagent from which they were obtained (Ames strain of Bacillus anthracis) and the molecular masses of the calibration amplicons provided the means for their identification as well. The relationship between the abundance (peak height) of the calibration amplicon signals and the bacterial bioagent identifying amplicon signals provides the means of calculation of the copies of the pX02 plasmid of the Ames strain of Bacillus anthracis. Methods of calculating quantities of molecules based on internal calibration procedures are well known to those of ordinary skill in the art.

Averaging the results of 10 repetitions of the experiment described above, enabled a calculation that indicated that the quantity of Ames strain of Bacillus anthracis present in the sample corresponds to approximately 10 copies of pX02 plasmid.

### Example 10

### Triangulation Genotyping Analysis of Campylobacter Species

A series of triangulation genotyping analysis primers were configured as described in Example 1 with the objective of identification of different strains of Campylobacter jejuni. The primers are listed in Table 12 with the designation “CJST_CJ.” Housekeeping genes to which the primers hybridize and produce bioagent identifying amplicons include: tkt (transketolase), glyA (serine hydroxymethyltransferase), gltA (citrate synthase), aspA (aspartate ammonia lyase), glnA (glutamine synthase), pgm (phosphoglycerate mutase), and uncA (ATP synthetase alpha chain).

TABLE 12
Campylobacter Genotyping Primer Pairs

| | | Forward | | Reverse | |
|---|---|---|---|---|---|
| Primer | | Primer | | Primer | Target |
| Pair No. | Forward Primer Name | (SEQ ID NO:) | Reverse Primer Name | (SEQ ID NO:) | Gene |
| 1053 | CJST_CJ_1080_1110_F | 681 | CJST_CJ_1166_1198_R | 1022 | gltA |
| 1047 | CJST_CJ_584_616_F | 315 | CJST_CJ_663_692_R | 1379 | glnA |
| 1048 | CJST_CJ_360_394_F | 346 | CJST_CJ_442_476_R | 955 | aspA |
| 1049 | CJST_CJ_2636_2668_F | 504 | CJST_CJ_2753_2777_R | 1409 | tkt |
| 1054 | CJST_CJ_2060_2090_F | 323 | CJST_CJ_2148_2174_R | 1068 | pgm |
| 1064 | CJST_CJ_1680_1713_F | 479 | CJST_CJ_1795_1822_R | 938 | glyA |

The primers were used to amplify nucleic acid from 50 food product samples provided by the USDA, 25 of which contained Campylobacter jejuni and 25 of which contained Campylobacter coli. Primers used in this study were developed primarily for the discrimination of Campylobacter jejuni clonal complexes and for distinguishing Campylobacter jejuni from Campylobacter coli. Finer discrimination between Campylobacter coli types is also possible by using specific primers targeted to loci where closely-related Campylobacter coli isolates demonstrate polymorphisms between strains. The conclusions of the comparison of base composition analysis with sequence analysis are shown in Tables 13A-C.

TABLE 13A
Results of Base Composition Analysis of 50 Campylobacter Samples with Drill-
down MLST Primer Pair Nos: 1048 and 1047

| | | | | | | Base | Base |
|---|---|---|---|---|---|---|---|
| | | | | | | Composition | Composition |
| | | | | MLST | | of Bioagent | of Bioagent |
| | | | MLST type | Type or | | Identifying | Identifying |
| | | | or Clonal | Clonal | | Amplicon | Amplicon |
| | | | Complex by | Complex | | Obtained with | Obtained with |
| | | | Base | by | | Primer Pair | Primer Pair |
| | | Isolate | Composition | Sequence | | No: 1048 | No: 1047 |
| Group | Species | origin | analysis | analysis | Strain | (aspA) | (glnA) |
| J-1 | C. jejuni | Goose | ST 690/ | ST 991 | RM3673 | A30 G25 C16 | A47 G21 C16 |
| | | | 692/707/991 | | | T46 | T25 |
| J-2 | C. jejuni | Human | Complex | ST 356, | RM4192 | A30 G25 C16 | A48 G21 C17 |
| | | | 206/48/353 | complex | | T46 | T23 |
| | | | | 353 | | | |
| J-3 | C. jejuni | Human | Complex | ST 436 | RM4194 | A30 G25 C15 | A48 G21 C18 |
| | | | 354/179 | | | T47 | T22 |
| J-4 | C. jejuni | Human | Complex 257 | ST 257, | RM4197 | A30 G25 C16 | A48 G21 C18 |
| | | | | complex | | T46 | T22 |
| | | | | 257 | | | |
| J-5 | C. jejuni | Human | Complex 52 | ST 52, | RM4277 | A30 G25 C16 | A48 G21 C17 |
| | | | | complex | | T46 | T23 |
| | | | | 52 | | | |
| J-6 | C. jejuni | Human | Complex 443 | ST 51, | RM4275 | A30 G25 C15 | A48 G21 C17 |
| | | | | complex | | T47 | T23 |
| | | | | 443 | RM4279 | A30 G25 C15 | A48 G21 C17 |
| | | | | | | T47 | T23 |
| J-7 | C. jejuni | Human | Complex 42 | ST 604, | RM1864 | A30 G25 C15 | A48 G21 C18 |
| | | | | complex | | T47 | T22 |
| | | | | 42 | | | |
| J-8 | C. jejuni | Human | Complex | ST 362, | RM3193 | A30 G25 C15 | A48 G21 C18 |
| | | | 42/49/362 | complex | | T47 | T22 |
| | | | | 362 | | | |
| J-9 | C. jejuni | Human | Complex | ST 147, | RM3203 | A30 G25 C15 | A47 G21 C18 |
| | | | 45/283 | Complex | | T47 | T23 |
| | | | | 45 | | | |
| | C. jejuni | Human | Consistent | ST 828 | RM4183 | A31 G27 C20 | A48 G21 C16 |
| C-1 | C. coli | | with 74 | | | T39 | T24 |
| | | | closely | ST 832 | RM1169 | A31 G27 C20 | A48 G21 C16 |
| | | | related | | | T39 | T24 |
| | | | sequence | ST 1056 | RM1857 | A31 G27 C20 | A48 G21 C16 |
| | | | types (none | | | T39 | T24 |
| | | Poultry | belong to a | ST 889 | RM1166 | A31 G27 C20 | A48 G21 C16 |
| | | | clonal | | | T39 | T24 |
| | | | complex) | ST 829 | RM1182 | A31 G27 C20 | A48 G21 C16 |
| | | | | | | T39 | T24 |
| | | | | ST 1050 | RM1518 | A31 G27 C20 | A48 G21 C16 |
| | | | | | | T39 | T24 |
| | | | | ST 1051 | RM1521 | A31 G27 C20 | A48 G21 C16 |
| | | | | | | T39 | T24 |
| | | | | ST 1053 | RM1523 | A31 G27 C20 | A48 G21 C16 |
| | | | | | | T39 | T24 |
| | | | | ST 1055 | RM1527 | A31 G27 C20 | A48 G21 C16 |
| | | | | | | T39 | T24 |
| | | | | ST 1017 | RM1529 | A31 G27 C20 | A48 G21 C16 |
| | | | | | | T39 | T24 |
| | | | | ST 860 | RM1840 | A31 G27 C20 | A48 G21 C16 |
| | | | | | | T39 | T24 |
| | | | | ST 1063 | RM2219 | A31 G27 C20 | A48 G21 C16 |
| | | | | | | T39 | T24 |
| | | | | ST 1066 | RM2241 | A31 G27 C20 | A48 G21 C16 |
| | | | | | | T39 | T24 |
| | | | | ST 1067 | RM2243 | A31 G27 C20 | A48 G21 C16 |
| | | | | | | T39 | T24 |
| | | | | ST 1068 | RM2439 | A31 G27 C20 | A48 G21 C16 |
| | | | | | | T39 | T24 |
| | | Swine | | ST 1016 | RM3230 | A31 G27 C20 | A48 G21 C16 |
| | | | | | | T39 | T24 |
| | | | | ST 1069 | RM3231 | A31 G27 C20 | A48 G21 C16 |
| | | | | | | T39 | T24 |
| | | | | ST 1061 | RM1904 | A31 G27 C20 | A48 G21 C16 |
| | | | | | | T39 | T24 |
| | | Unknown | | ST 825 | RM1534 | A31 G27 C20 | A48 G21 C16 |
| | | | | | | T39 | T24 |
| | | | | ST 901 | RM1505 | A31 G27 C20 | A48 G21 C16 |
| | | | | | | T39 | T24 |
| C-2 | C. coli | Human | ST 895 | ST 895 | RM1532 | A31 G27 C19 | A48 G21 C16 |
| | | | | | | T40 | T24 |
| C-3 | C. coli | Poultry | Consistent | ST 1064 | RM2223 | A31 G27 C20 | A48 G21 C16 |
| | | | with 63 | | | T39 | T24 |
| | | | closely | ST 1082 | RM1178 | A31 G27 C20 | A48 G21 C16 |
| | | | related | | | T39 | T24 |
| | | | sequence | ST 1054 | RM1525 | A31 G27 C20 | A48 G21 C16 |
| | | | types (none | | | T39 | T24 |
| | | | belong to a | ST 1049 | RM1517 | A31 G27 C20 | A48 G21 C16 |
| | | | clonal | | | T39 | T24 |
| | | Marmoset | complex) | ST 891 | RM1531 | A31 G27 C20 | A48 G21 C16 |
| | | | | | | T39 | T24 |

TABLE 13B
Results of Base Composition Analysis of 50 Campylobacter Samples with Drill-
down MLST Primer Pair Nos: 1053 and 1064

| | | | | | | Base | Base |
|---|---|---|---|---|---|---|---|
| | | | | | | Composition | Composition |
| | | | | MLST | | of Bioagent | of Bioagent |
| | | | MLST type | Type or | | Identifying | Identifying |
| | | | or Clonal | Clonal | | Amplicon | Amplicon |
| | | | Complex by | Complex | | Obtained with | Obtained with |
| | | | Base | by | | Primer Pair | Primer Pair |
| | | Isolate | Composition | Sequence | | No: 1053 | No: 1064 |
| Group | Species | origin | analysis | analysis | Strain | (gltA) | (glyA) |
| J-1 | C. jejuni | Goose | ST 690/ | ST 991 | RM3673 | A24 G25 C23 | A40 G29 C29 |
| | | | 692/707/991 | | T47 | T45 | |
| J-2 | C. jejuni | Human | Complex | ST 356, | RM4192 | A24 G25 C23 | A40 G29 C29 |
| | | | 206/48/353 | complex | | T47 | T45 |
| | | | | 353 | | | |
| J-3 | C. jejuni | Human | Complex | ST 436 | RM4194 | A24 G25 C23 | A40 G29 C29 |
| | | | 354/179 | | | T47 | T45 |
| J-4 | C. jejuni | Human | Complex 257 | ST 257, | RM4197 | A24 G25 C23 | A40 G29 C29 |
| | | | | complex | | T47 | T45 |
| | | | | 257 | | | |
| J-5 | C. jejuni | Human | Complex 52 | ST 52, | RM4277 | A24 G25 C23 | A39 G30 C26 |
| | | | | complex | | T47 | T48 |
| | | | | 52 | | | |
| J-6 | C. jejuni | Human | Complex 443 | ST 51, | RM4275 | A24 G25 C23 | A39 G30 C28 |
| | | | | complex | | T47 | T46 |
| | | | | 443 | RM4279 | A24 G25 C23 | A39 G30 C28 |
| | | | | | | T47 | T46 |
| J-7 | C. jejuni | Human | Complex 42 | ST 604, | RM1864 | A24 G25 C23 | A39 G30 C26 |
| | | | | complex | | T47 | T48 |
| | | | | 42 | | | |
| J-8 | C. jejuni | Human | Complex | ST 362, | RM3193 | A24 G25 C23 | A38 G31 C28 |
| | | | 42/49/362 | complex | | T47 | T46 |
| | | | | 362 | | | |
| J-9 | C. jejuni | Human | Complex | ST 147, | RM3203 | A24 G25 C23 | A38 G31 C28 |
| | | | 45/283 | Complex | | T47 | T46 |
| | | | | 45 | | | |
| | C. jejuni | Human | Consistent | ST 828 | RM4183 | A23 G24 C26 | A39 G30 C27 |
| C-1 | C. coli | | with 74 | | | T46 | T47 |
| | | | closely | ST 832 | RM1169 | A23 G24 C26 | A39 G30 C27 |
| | | | related | | | T46 | T47 |
| | | | sequence | ST 1056 | RM1857 | A23 G24 C26 | A39 G30 C27 |
| | | | types (none | | | T46 | T47 |
| | | Poultry | belong to a | ST 889 | RM1166 | A23 G24 C26 | A39 G30 C27 |
| | | | clonal | | | T46 | T47 |
| | | | complex) | ST 829 | RM1182 | A23 G24 C26 | A39 G30 C27 |
| | | | | | | T46 | T47 |
| | | | | ST 1050 | RM1518 | A23 G24 C26 | A39 G30 C27 |
| | | | | | | T46 | T47 |
| | | | | ST 1051 | RM1521 | A23 G24 C26 | A39 G30 C27 |
| | | | | | | T46 | T47 |
| | | | | ST 1053 | RM1523 | A23 G24 C26 | A39 G30 C27 |
| | | | | | | T46 | T47 |
| | | | | ST 1055 | RM1527 | A23 G24 C26 | A39 G30 C27 |
| | | | | | | T46 | T47 |
| | | | | ST 1017 | RM1529 | A23 G24 C26 | A39 G30 C27 |
| | | | | | | T46 | T47 |
| | | | | ST 860 | RM1840 | A23 G24 C26 | A39 G30 C27 |
| | | | | | | T46 | T47 |
| | | | | ST 1063 | RM2219 | A23 G24 C26 | A39 G30 C27 |
| | | | | | | T46 | T47 |
| | | | | ST 1066 | RM2241 | A23 G24 C26 | A39 G30 C27 |
| | | | | | | T46 | T47 |
| | | | | ST 1067 | RM2243 | A23 G24 C26 | A39 G30 C27 |
| | | | | | | T46 | T47 |
| | | | | ST 1068 | RM2439 | A23 G24 C26 | A39 G30 C27 |
| | | | | | | T46 | T47 |
| | | Swine | | ST 1016 | RM3230 | A23 G24 C26 | A39 G30 C27 |
| | | | | | | T46 | T47 |
| | | | | ST 1069 | RM3231 | A23 G24 C26 | NO DATA |
| | | | | | | T46 | |
| | | | | ST 1061 | RM1904 | A23 G24 C26 | A39 G30 C27 |
| | | | | | | T46 | T47 |
| | | Unknown | | ST 825 | RM1534 | A23 G24 C26 | A39 G30 C27 |
| | | | | | | T46 | T47 |
| | | | | ST 901 | RM1505 | A23 G24 C26 | A39 G30 C27 |
| | | | | | | T46 | T47 |
| C-2 | C. coli | Human | ST 895 | ST 895 | RM1532 | A23 G24 C26 | A39 G30 C27 |
| | | | | | | T46 | T47 |
| C-3 | C. coli | Poultry | Consistent | ST 1064 | RM2223 | A23 G24 C26 | A39 G30 C27 |
| | | | with 63 | | | T46 | T47 |
| | | | closely | ST 1082 | RM1178 | A23 G24 C26 | A39 G30 C27 |
| | | | related | | | T46 | T47 |
| | | | sequence | ST 1054 | RM1525 | A23 G24 C25 | A39 G30 C27 |
| | | | types (none | | | T47 | T47 |
| | | | belong to a | ST 1049 | RM1517 | A23 G24 C26 | A39 G30 C27 |
| | | | clonal | | | T46 | T47 |
| | | Marmoset | complex) | ST 891 | RM1531 | A23 G24 C26 | A39 G30 C27 |
| | | | | | | T46 | T47 |

TABLE 13C
Results of Base Composition Analysis of 50 Campylobacter Samples with Drill-
down MLST Primer Pair Nos: 1054 and 1049

| | | | | | | Base | Base |
|---|---|---|---|---|---|---|---|
| | | | | | | Composition | Composition |
| | | | | MLST | | of Bioagent | of Bioagent |
| | | | MLST type | Type or | | Identifying | Identifying |
| | | | or Clonal | Clonal | | Amplicon | Amplicon |
| | | | Complex by | Complex | | Obtained with | Obtained with |
| | | | Base | by | | Primer Pair | Primer Pair |
| | | Isolate | Composition | Sequence | | No: 1054 | No: 1049 |
| Group | Species | origin | analysis | analysis | Strain | (pgm) | (tkt) |
| J-1 | C. jejuni | Goose | ST 690/ | ST 991 | RM3673 | A26 G33 C18 | A41 G28 C35 |
| | | | 692/707/991 | | | T38 | T38 |
| J-2 | C. jejuni | Human | Complex | ST 356, | RM4192 | A26 G33 C19 | A41 G28 C36 |
| | | | 206/48/353 | complex | | T37 | T37 |
| | | | | 353 | | | |
| J-3 | C. jejuni | Human | Complex | ST 436 | RM4194 | A27 G32 C19 | A42 G28 C36 |
| | | | 354/179 | | | T37 | T36 |
| J-4 | C. jejuni | Human | Complex 257 | ST 257, | RM4197 | A27 G32 C19 | A41 G29 C35 |
| | | | | complex | | T37 | T37 |
| | | | | 257 | | | |
| J-5 | C. jejuni | Human | Complex 52 | ST 52, | RM4277 | A26 G33 C18 | A41 G28 C36 |
| | | | | complex | | T38 | T37 |
| | | | | 52 | | | |
| J-6 | C. jejuni | Human | Complex 443 | ST 51, | RM4275 | A27 G31 C19 | A41 G28 C36 |
| | | | | complex | | T38 | T37 |
| | | | | 443 | RM4279 | A27 G31 C19 | A41 G28 C36 |
| | | | | | | T38 | T37 |
| J-7 | C. jejuni | Human | Complex 42 | ST 604, | RM1864 | A27 G32 C19 | A42 G28 C35 |
| | | | | complex | | T37 | T37 |
| | | | | 42 | | | |
| J-8 | C. jejuni | Human | Complex | ST 362, | RM3123 | A26 G33 C19 | A42 G28 C35 |
| | | | 42/49/362 | complex | | T37 | T37 |
| | | | | 362 | | | |
| J-9 | C. jejuni | Human | Complex | ST 147, | RM3203 | A28 G31 C19 | A43 G28 C36 |
| | | | 45/283 | Complex | | T37 | T35 |
| | | | | 45 | | | |
| | C. jejuni | Human | Consistent | ST 828 | RM4183 | A27 G30 C19 | A46 G28 C32 |
| C-1 | C. coli | | with 74 | | | T39 | T36 |
| | | | closely | ST 832 | RM1169 | A27 G30 C19 | A46 G28 C32 |
| | | | related | | | T39 | T36 |
| | | | sequence | ST 1056 | RM1857 | A27 G30 C19 | A46 G28 C32 |
| | | | types (none | | | T39 | T36 |
| | | Poultry | belong to a | ST 889 | RM1166 | A27 G30 C19 | A46 G28 C32 |
| | | | clonal | | | T39 | T36 |
| | | | complex) | ST 829 | RM1182 | A27 G30 C19 | A46 G28 C32 |
| | | | | | | T39 | T36 |
| | | | | ST 1050 | RM1518 | A27 G30 C19 | A46 G28 C32 |
| | | | | | | T39 | T36 |
| | | | | ST 1051 | RM1521 | A27 G30 C19 | A46 G28 C32 |
| | | | | | | T39 | T36 |
| | | | | ST 1053 | RM1523 | A27 G30 C19 | A46 G28 C32 |
| | | | | | | T39 | T36 |
| | | | | ST 1055 | RM1527 | A27 G30 C19 | A46 G28 C32 |
| | | | | | | T39 | T36 |
| | | | | ST 1017 | RM1529 | A27 G30 C19 | A46 G28 C32 |
| | | | | | | T39 | T36 |
| | | | | ST 860 | RM1840 | A27 G30 C19 | A46 G28 C32 |
| | | | | | | T39 | T36 |
| | | | | ST 1063 | RM2219 | A27 G30 C19 | A46 G28 C32 |
| | | | | | | T39 | T36 |
| | | | | ST 1066 | RM2241 | A27 G30 C19 | A46 G28 C32 |
| | | | | | | T39 | T36 |
| | | | | ST 1067 | RM2243 | A27 G30 C19 | A46 G28 C32 |
| | | | | | | T39 | T36 |
| | | | | ST 1068 | RM2439 | A27 G30 C19 | A46 G28 C32 |
| | | | | | | T39 | T36 |
| | | Swine | | ST 1016 | RM3230 | A27 G30 C19 | A46 G28 C32 |
| | | | | | | T39 | T36 |
| | | | | ST 1069 | RM3231 | A27 G30 C19 | A46 G28 C32 |
| | | | | | | T39 | T36 |
| | | | | ST 1061 | RM1904 | A27 G30 C19 | A46 G28 C32 |
| | | | | | | T39 | T36 |
| | | Unknown | | ST 825 | RM1534 | A27 G30 C19 | A46 G28 C32 |
| | | | | | | T39 | T36 |
| | | | | ST 901 | RM1505 | A27 G30 C19 | A46 G28 C32 |
| | | | | | | T39 | T36 |
| C-2 | C. coli | Human | ST 895 | ST 895 | RM1532 | A27 G30 C19 | A45 G29 C32 |
| | | | | | | T39 | T36 |
| C-3 | C. coli | Poultry | Consistent | ST 1064 | RM2223 | A27 G30 C19 | A45 G29 C32 |
| | | | with 63 | | | T39 | T36 |
| | | | closely | ST 1082 | RM1178 | A27 G30 C19 | A45 G29 C32 |
| | | | related | | | T39 | T36 |
| | | | sequence | ST 1054 | RM1525 | A27 G30 C19 | A45 G29 C32 |
| | | | types (none | | | T39 | T36 |
| | | | belong to a | ST 1049 | RM1517 | A27 G30 C19 | A45 G29 C32 |
| | | | clonal | | | T39 | T36 |
| | | Marmoset | complex) | ST 891 | RM1531 | A27 G30 C19 | A45 G29 C32 |
| | | | | | | T39 | T36 |

The base composition analysis method was successful in identification of 12 different strain groups. Campylobacter jejuni and Campylobacter coli are generally differentiated by all loci. Ten clearly differentiated Campylobacter jejuni isolates and 2 major Campylobacter coli groups were identified even though the primers were configured for strain typing of Campylobacter jejuni. One isolate (RM4183) which was designated as Campylobacter jejuni was found to group with Campylobacter coli and also appears to actually be Campylobacter coli by full MLST sequencing.

### Example 11

### Identification of Acinetobacter baumannii Using Broad Range Survey and Division-Wide Primers in Epidemiological Surveillance

To test the capability of the broad range survey and division-wide primer sets of Table 5 in identification of Acinetobacter species, 183 clinical samples were obtained from individuals participating in, or in contact with individuals participating in Operation Iraqi Freedom (including US service personnel, US civilian patients at the Walter Reed Army Institute of Research (WRAIR), medical staff, Iraqi civilians and enemy prisoners. In addition, 34 environmental samples were obtained from hospitals in Iraq, Kuwait, Germany, the United States and the USNS Comfort, a hospital ship.

Upon amplification of nucleic acid obtained from the clinical samples, primer pairs 346-349, 360, 361, 354, 362 and 363 (Table 5) all produced bacterial bioagent amplicons which identified Acinetobacter baumannii in 215 of 217 samples. The organism Klebsiella pneumoniae was identified in the remaining two samples. In addition, 14 different strain types (containing single nucleotide polymorphisms relative to a reference strain of Acinetobacter baumannii) were identified and assigned arbitrary numbers from 1 to 14. Strain type 1 was found in 134 of the sample isolates and strains 3 and 7 were found in 46 and 9 of the isolates respectively.

The epidemiology of strain type 7 of Acinetobacter baumannii was investigated. Strain 7 was found in 4 patients and 5 environmental samples (from field hospitals in Iraq and Kuwait). The index patient infected with strain 7 was a pre-war patient who had a traumatic amputation in March of 2003 and was treated at a Kuwaiti hospital. The patient was subsequently transferred to a hospital in Germany and then to WRAIR. Two other patients from Kuwait infected with strain 7 were found to be non-infectious and were not further monitored. The fourth patient was diagnosed with a strain 7 infection in September of 2003 at WRAIR. Since the fourth patient was not related involved in Operation Iraqi Freedom, it was inferred that the fourth patient was the subject of a nosocomial infection acquired at WRAIR as a result of the spread of strain 7 from the index patient.

The epidemiology of strain type 3 of Acinetobacter baumannii was also investigated. Strain type 3 was found in 46 samples, all of which were from patients (US service members, Iraqi civilians and enemy prisoners) who were treated on the USNS Comfort hospital ship and subsequently returned to Iraq or Kuwait. The occurrence of strain type 3 in a single locale may provide evidence that at least some of the infections at that locale were a result of nosocomial infections.

This example thus illustrates an embodiment of the present invention wherein the methods of analysis of bacterial bioagent identifying amplicons provide the means for epidemiological surveillance.

### Example 12

### Selection and Use of Triangulation Genotyping Analysis Primer Pairs for Acinetobacter baumanii

To combine the power of high-throughput mass spectrometric analysis of bioagent identifying amplicons with the sub-species characteristic resolving power provided by triangulation genotyping analysis, an additional 21 primer pairs were selected based on analysis of housekeeping genes of the genus Acinetobacter. Genes to which the drill-down triangulation genotyping analysis primers hybridize for production of bacterial bioagent identifying amplicons include anthranilate synthase component I (trpE), adenylate kinase (adk), adenine glycosylase (mutY), fumarate hydratase (fumC), and pyrophosphate phospho-hydratase (ppa). These 21 primer pairs are indicated with reference to sequence listings in Table 14. Primer pair numbers 1151-1154 hybridize to and amplify segments of trpE. Primer pair numbers 1155-1157 hybridize to and amplify segments of adk. Primer pair numbers 1158-1164 hybridize to and amplify segments of mutY. Primer pair numbers 1165-1170 hybridize to and amplify segments of fumC. Primer pair number 1171 hybridizes to and amplifies a segment of ppa. Primer pair numbers: 2846-2848 hybridize to and amplify segments of the parC gene of DNA topoisomerase which include a codon known to confer quinolone drug resistance upon sub-types of Acinetobacter baumannii. Primer pair numbers 2852-2854 hybridize to and amplify segments of the gyrA gene of DNA gyrase which include a codon known to confer quinolone drug resistance upon sub-types of Acinetobacter baumannii. Primer pair numbers 2922 and 2972 are speciating primers which are useful for identifying different species members of the genus Acinetobacter. The primer names given in Table 14A (with the exception of primer pair numbers 2846-2848, 2852-2854) indicate the coordinates to which the primers hybridize to a reference sequence which comprises a concatenation of the genes TrpE, efp (elongation factor p), adk, mutT, fumC, and ppa. For example, the forward primer of primer pair 1151 is named AB_MLST-11-OIF007_{—}62_{—}91_F because it hybridizes to the Acinetobacter primer reference sequence of strain type 11 in sample 007 of Operation Iraqi Freedom (OIF) at positions 62 to 91. DNA was sequenced from strain type 11 and from this sequence data and an artificial concatenated sequence of partial gene extractions was assembled for use in design of the triangulation genotyping analysis primers. The stretches of arbitrary residues “N”s in the concatenated sequence were added for the convenience of separation of the partial gene extractions (40N for AB_MLST (SEQ ID NO: 1444)).

The hybridization coordinates of primer pair numbers 2846-2848 are with respect to GenBank Accession number X95819. The hybridization coordinates of primer pair numbers 2852-2854 are with respect to GenBank Accession number AY642140. Sequence residue “I” appearing in the forward and reverse primers of primer pair number 2972 represents inosine.

TABLE 14A
Triangulation Genotyping Analysis Primer Pairs for Identification of Sub-species
characteristics (Strain Type) of Members of the Bacterial Genus Acinetobacter

| | | Forward | | Reverse |
|---|---|---|---|---|
| Primer | | Primer (SEQ | | Primer (SEQ |
| Pair No. | Forward Primer Name | ID NO:) | Reverse Primer Name | ID NO:) |
| 1151 | AB_MLST-11- | 454 | AB_MLST-11- | 1418 |
| | OIF007_62_91_F | | OIF007_169_203_R | |
| 1152 | AB_MLST-11- | 243 | AB_MLST-11- | 969 |
| | OIF007_185_214_F | | OIF007_291_324_R | |
| 1153 | AB_MLST-11- | 541 | AB_MLST-11- | 1400 |
| | OIF007_260_289_F | | OIF007_364_393_R | |
| 1154 | AB_MLST-11- | 436 | AB_MLST-11- | 1036 |
| | OIF007_206_239_F | | OIF007_318_344_R | |
| 1155 | AB_MLST-11- | 378 | AB_MLST-11- | 1392 |
| | OIF007_522_552_F | | OIF007_587_610_R | |
| 1156 | AB_MLST-11- | 250 | AB_MLST-11- | 902 |
| | OIF007_547_571_F | | OIF007_656_686_R | |
| 1157 | AB_MLST-11- | 256 | AB_MLST-11- | 881 |
| | OIF007_601_627_F | | OIF007_710_736_R | |
| 1158 | AB_MLST-11- | 384 | AB_MLST-11- | 878 |
| | OIF007_1202_1225_F | | OIF007_1266_1296_R | |
| 1159 | AB_MLST-11- | 384 | AB_MLST-11- | 1199 |
| | OIF007_1202_1225_F | | OIF007_1299_1316_R | |
| 1160 | AB_MLST-11- | 694 | AB_MLST-11- | 1215 |
| | OIF007_1234_1264_F | | OIF007_1335_1362_R | |
| 1161 | AB_MLST-11- | 225 | AB_MLST-11- | 1212 |
| | OIF007_1327_1356_F | | OIF007_1422_1448_R | |
| 1162 | AB_MLST-11- | 383 | AB_MLST-11- | 1083 |
| | OIF007_1345_1369_F | | OIF007_1470_1494_R | |
| 1163 | AB_MLST-11- | 662 | AB_MLST-11- | 1083 |
| | OIF007_1351_1375_F | | OIF007_1470_1494_R | |
| 1164 | AB_MLST-11- | 422 | AB_MLST-11- | 1083 |
| | OIF007_1387_1412_F | | OIF007_1470_1494_R | |
| 1165 | AB_MLST-11- | 194 | AB_MLST-11- | 1173 |
| | OIF007_1542_1569_F | | OIF007_1656_1680_R | |
| 1166 | AB_MLST-11- | 684 | AB_MLST-11- | 1173 |
| | OIF007_1566_1593_F | | OIF007_1656_1680_R | |
| 1167 | AB_MLST-11- | 375 | AB_MLST-11- | 890 |
| | OIF007_1611_1638_F | | OIF007_1731_1757_R | |
| 1168 | AB_MLST-11- | 182 | AB_MLST-11- | 1195 |
| | OIF007_1726_1752_F | | OIF007_1790_1821_R | |
| 1169 | AB_MLST-11- | 656 | AB_MLST-11- | 1151 |
| | OIF007_1792_1826_F | | OIF007_1876_1909_R | |
| 1170 | AB_MLST-11- | 656 | AB_MLST-11- | 1224 |
| | OIF007_1792_1826_F | | OIF007_1895_1927_R | |
| 1171 | AB_MLST-11- | 618 | AB_MLST-11- | 1157 |
| | OIF007_1970_2002_F | | OIF007_2097_2118_R | |
| 2846 | PARC_X95819_33_58_F | 302 | PARC_X95819_121_153_R | 852 |
| 2847 | PARC_X95819_33_58_F | 199 | PARC_X95819_157_178_R | 889 |
| 2848 | PARC_X95819_33_58_F | 596 | PARC_X95819_97_128_R | 1169 |
| 2852 | GYRA_AY642140_-1_24_F | 150 | GYRA_AY642140_71_100_R | 1242 |
| 2853 | GYRA_AY642140_26_54_F | 166 | GYRA_AY642140_121_146_R | 1069 |
| 2854 | GYRA_AY642140_26_54_F | 166 | GYRA_AY642140_58_89_R | 1168 |
| 2922 | AB_MLST-11- | 583 | AB_MLST-11- | 923 |
| | OIF007_991_1018_F | | OIF007_1110_1137_R | |
| 2972 | AB_MLST-11- | 592 | AB_MLST-11- | 924 |
| | OIF007_1007_1034_F | | OIF007_1126_1153_R | |

TABLE 14B

Triangulation Genotyping Analysis Primer Pairs for Identification of Sub-species
characteristics (Strain Type) of Members of the Bacterial Genus Acinetobacter

| | | | | |
|---|---|---|---|---|
| | Forward | | Reverse | |
| Primer | Primer | | Primer | |
| Pair No. | (SEQ ID NO:) | SEQUENCE | (SEQ ID NO:) | SEQUENCE |

| | | | | |
|---|---|---|---|---|
| 1151 | 454 | TGAGATTGCTGAACATTTAATGCTGATTGA | 1418 | TTGTACATTTGAAACAATATGCATGACATGTGA |
| | | | | AT |
| 1152 | 243 | TATTGTTTCAAATGTACAAGGTGAAGTGCG | 969 | TCACAGGTTCTACTTCATCAATAATTTCCAT |
| | | | | TGC |
| 1153 | 541 | TGGAACGTTATCAGGTGCCCCAAAAATTCG | 1400 | TTGCAATCGACATATCCATTTCACCATGCC |
| 1154 | 436 | TGAAGTGCGTGATGATATCGATGCACTTGATGTA | 1036 | TCCGCCAAAAACTCCCCTTTTCACAGG |
| 1155 | 378 | TCGGTTTAGTAAAAGAACGTATTGCTCAACC | 1392 | TTCTGCTTGAGGAATAGTGCGTGG |
| 1156 | 250 | TCAACCTGACTGCGTGAATGGTTGT | 902 | TACGTTCTACGATTTCTTCATCAGGTACATC |
| 1157 | 256 | TCAAGCAGAAGCTTTGGAAGAAGAAGG | 881 | TACAACGTGATAAACACGACCAGAAGC |
| 1158 | 384 | TCGTGCCCGCAATTTGCATAAAGC | 878 | TAATGCCGGGTAGTGCAATCCATTCTTCTAG |
| 1159 | 384 | TCGTGCCCGCAATTTGCATAAAGC | 1199 | TGCACCTGCGGTCGAGCG |
| 1160 | 694 | TTGTAGCACAGCAAGGCAAATTTCCTGAAAC | 1215 | TGCCATCCATAATCACGCCATACTGACG |
| 1161 | 225 | TAGGTTTACGTCAGTATGGCGTGATTATGG | 1212 | TGCCAGTTTCCACATTTCACGTTCGTG |
| 1162 | 383 | TCGTGATTATGGATGGCAACGTGAA | 1083 | TCGCTTGAGTGTAGTCATGATTGCG |
| 1163 | 662 | TTATGGATGGCAACGTGAAACGCGT | 1083 | TCGCTTGAGTGTAGTCATGATTGCG |
| 1164 | 422 | TCTTTGCCATTGAAGATGACTTAAGC | 1083 | TCGCTTGAGTGTAGTCATGATTGCG |
| 1165 | 194 | TACTAGCGGTAAGCTTAAACAAGATTGC | 1173 | TGAGTCGGGTTCACTTTACCTGGCA |
| 1166 | 684 | TTGCCAATGATATTCGTTGGTTAGCAAG | 1173 | TGAGTCGGGTTCACTTTACCTGGCA |
| 1167 | 375 | TCGGCGAAATCCGTATTCCTGAAAATGA | 890 | TACCGGAAGCACCAGCGACATTAATAG |
| 1168 | 182 | TACCACTATTAATGTCGCTGGTGCTTC | 1195 | TGCAACTGAATAGATTGCAGTAAGTTATAAGC |
| 1169 | 656 | TTATAACTTACTGCAATCTATTCAGTTGCTT | 1151 | TGAATTATGCAAGAAGTGATCAATTTTCTCA |
| | | GGTG | | CGA |
| 1170 | 656 | TTATAACTTACTGCAATCTATTCAGTTGCTT | 1224 | TGCCGTAACTAACATAAGAGAATTATGCAAG |
| | | GGTG | | AA |
| 1171 | 618 | TGGTTATGTACCAAATACTTTGTCTGAAGAT | 1157 | TGACGGCATCGATACCACCGTC |
| | | GG | | |
| 2846 | 302 | TCCAAAAAAATCAGCGCGTACAGTGG | 852 | TAAAGGATAGCGGTAACTAAATGGCTGAGCC |
| | | | | AT |
| 2847 | 199 | TACTTGGTAAATACCACCCACATGGTGA | 889 | TACCCCAGTTCCCCTGACCTTC |
| 2848 | 596 | TGGTAAATACCACCCACATGGTGAC | 1169 | TGAGCCATGAGTACCATGGCTTCATAACATGC |
| 2852 | 150 | TAAATCTGCCCGTGTCGTTGGTGAC | 1242 | TGCTAAAGTCTTGAGCCATACGAACAATGG |
| 2853 | 166 | TAATCGGTAAATATCACCCGCATGGTGAC | 1069 | TCGATCGAACCGAAGTTACCCTGACC |
| 2854 | 166 | TAATCGGTAAATATCACCCGCATGGTGAC | 1168 | TGAGCCATACGAACAATGGTTTCATAAACAGC |
| 2922 | 583 | TGGGCGATGCTGCGAAATGGTTAAAAGA | 923 | TAGTATCACCACGTACACCCGGATCAGT |
| 2972 | 592 | TGGGIGATGCTGCIAAATGGTTAAAAGA | 924 | TAGTATCACCACGTACICCIGGATCAGT |

Analysis of bioagent identifying amplicons obtained using the primers of Table 14B for over 200 samples from Operation Iraqi Freedom resulted in the identification of 50 distinct strain type clusters. The largest cluster, designated strain type 11 (ST11) includes 42 sample isolates, all of which were obtained from US service personnel and Iraqi civilians treated at the 28^{th} Combat Support Hospital in Baghdad. Several of these individuals were also treated on the hospital ship USNS Comfort. These observations are indicative of significant epidemiological correlation/linkage.

All of the sample isolates were tested against a broad panel of antibiotics to characterize their antibiotic resistance profiles. As an example of a representative result from antibiotic susceptibility testing, ST11 was found to consist of four different clusters of isolates, each with a varying degree of sensitivity/resistance to the various antibiotics tested which included penicillins, extended spectrum penicillins, cephalosporins, carbepenem, protein synthesis inhibitors, nucleic acid synthesis inhibitors, anti-metabolites, and anti-cell membrane antibiotics. Thus, the genotyping power of bacterial bioagent identifying amplicons, particularly drill-down bacterial bioagent identifying amplicons, has the potential to increase the understanding of the transmission of infections in combat casualties, to identify the source of infection in the environment, to track hospital transmission of nosocomial infections, and to rapidly characterize drug-resistance profiles which enable development of effective infection control measures on a time-scale previously not achievable.

### Example 13

### Triangulation Genotyping Analysis and Codon Analysis of Acinetobacter baumannii Samples from Two Health Care Facilities

In this investigation, 88 clinical samples were obtained from Walter Reed Hospital and 95 clinical samples were obtained from Northwestern Medical Center. All samples from both healthcare facilities were suspected of containing sub-types of Acinetobacter baumannii, at least some of which were expected to be resistant to quinolone drugs. Each of the 183 samples was analyzed by the method of the present invention. DNA was extracted from each of the samples and amplified with eight triangulation genotyping analysis primer pairs represented by primer pair numbers: 1151, 1156, 1158, 1160, 1165, 1167, 1170, and 1171. The DNA was also amplified with speciating primer pair number 2922 and codon analysis primer pair numbers 2846-2848 which interrogate a codon present in the parC gene, and primer pair numbers 2852-2854 which bracket a codon present in the gyrA gene. The parC and gyrA codon mutations are both responsible for causing drug resistance in Acinetobacter baumannii. During evolution of drug resistant strains, the gyrA mutation usually occurs before the parC mutation. Amplification products were measured by ESI-TOF mass spectrometry as indicated in Example 4. The base compositions of the amplification products were calculated from the average molecular masses of the amplification products and are shown in Tables 15-18. The entries in each of the tables are grouped according to strain type number, which is an arbitrary number assigned to Acinetobacter baumannii strains in the order of observance beginning from the triangulation genotyping analysis OIF genotyping study described in Example 12. For example, strain type 11 which appears in samples from the Walter Reed Hospital is the same strain as the strain type 11 mentioned in Example 12. Ibis# refers to the order in which each sample was analyzed. Isolate refers to the original sample isolate numbering system used at the location from which the samples were obtained (either Walter Reed Hospital or Northwestern Medical Center). ST=strain type. ND=not detected. Base compositions highlighted with bold type indicate that the base composition is a unique base composition for the amplification product obtained with the pair of primers indicated.

TABLE 15A
Base Compositions of Amplification Products of 88 A. baumannii Samples Obtained from
Walter Reed Hospital and Amplified with Codon Analysis Primer Pairs Targeting the gyrA Gene

| | | | | | | PP No: 2854 |
|---|---|---|---|---|---|---|
| Species | Ibis# | Isolate | ST | PP No: 2852 gyrA | PP No: 2853 gyrA | gyrA |
| A. baumannii | 20 | 1082 | 1 | A25G23C22T31 | A29G28C22T42 | A17G13C14T20 |
| A. baumannii | 13 | 854 | 10 | A25G23C21T32 | A29G28C21T43 | A17G13C13T21 |
| A. baumannii | 22 | 1162 | 10 | A25G23C21T32 | A29G28C21T43 | A17G13C13T21 |
| A. baumannii | 27 | 1230 | 10 | A25G23C21T32 | A29G28C21T43 | A17G13C13T21 |
| A. baumannii | 31 | 1367 | 10 | A25G23C21T32 | A29G28C21T43 | A17G13C13T21 |
| A. baumannii | 37 | 1459 | 10 | A25G23C21T32 | A29G28C21T43 | A17G13C13T21 |
| A. baumannii | 55 | 1700 | 10 | A25G23C21T32 | A29G28C21T43 | A17G13C13T21 |
| A. baumannii | 64 | 1777 | 10 | A25G23C21T32 | A29G28C21T43 | A17G13C13T21 |
| A. baumannii | 73 | 1861 | 10 | A25G23C21T32 | A29G28C21T43 | A17G13C13T21 |
| A. baumannii | 74 | 1877 | 10 | ND | A29G28C21T43 | A17G13C13T21 |
| A. baumannii | 86 | 1972 | 10 | A25G23C21T32 | A29G28C21T43 | A17G13C13T21 |
| A. baumannii | 3 | 684 | 11 | A25G23C21T32 | A29G28C21T43 | A17G13C13T21 |
| A. baumannii | 6 | 720 | 11 | A25G23C21T32 | A29G28C21T43 | A17G13C13T21 |
| A. baumannii | 7 | 726 | 11 | A25G23C21T32 | A29G28C21T43 | A17G13C13T21 |
| A. baumannii | 19 | 1079 | 11 | A25G23C21T32 | A29G28C21T43 | A17G13C13T21 |
| A. baumannii | 21 | 1123 | 11 | A25G23C21T32 | A29G28C21T43 | A17G13C13T21 |
| A. baumannii | 23 | 1188 | 11 | A25G23C21T32 | A29G28C21T43 | A17G13C13T21 |
| A. baumannii | 33 | 1417 | 11 | A25G23C21T32 | A29G28C21T43 | A17G13C13T21 |
| A. baumannii | 34 | 1431 | 11 | A25G23C21T32 | A29G28C21T43 | A17G13C13T21 |
| A. baumannii | 38 | 1496 | 11 | A25G23C21T32 | A29G28C21T43 | A17G13C13T21 |
| A. baumannii | 40 | 1523 | 11 | A25G23C21T32 | A29G28C21T43 | A17G13C13T21 |
| A. baumannii | 42 | 1640 | 11 | A25G23C21T32 | A29G28C21T43 | A17G13C13T21 |
| A. baumannii | 50 | 1666 | 11 | A25G23C21T32 | A29G28C21T43 | A17G13C13T21 |
| A. baumannii | 51 | 1668 | 11 | A25G23C21T32 | A29G28C21T43 | A17G13C13T21 |
| A. baumannii | 52 | 1695 | 11 | A25G23C21T32 | A29G28C21T43 | A17G13C13T21 |
| A. baumannii | 65 | 1781 | 11 | A25G23C21T32 | A29G28C21T43 | A17G13C13T21 |
| A. baumannii | 44 | 1649 | 12 | A25G23C21T32 | A29G28C21T43 | A17G13C13T21 |
| A. baumannii | 49A | 1658.1 | 12 | A25G23C22T31 | A29G28C21T43 | A17G13C13T21 |
| A. baumannii | 49B | 1658.2 | 12 | A25G23C21T32 | A29G28C21T43 | A17G13C13T21 |
| A. baumannii | 56 | 1707 | 12 | A25G23C21T32 | A29G28C21T43 | A17G13C13T21 |
| A. baumannii | 80 | 1893 | 12 | A25G23C21T32 | A29G28C21T43 | A17G13C13T21 |
| A. baumannii | 5 | 693 | 14 | A25G23C21T32 | A29G28C21T43 | A17G13C13T21 |
| A. baumannii | 8 | 749 | 14 | A25G23C21T32 | A29G28C21T43 | A17G13C13T21 |
| A. baumannii | 10 | 839 | 14 | A25G23C21T32 | A29G28C21T43 | A17G13C13T21 |
| A. baumannii | 14 | 865 | 14 | A25G23C21T32 | A29G28C21T43 | A17G13C13T21 |
| A. baumannii | 16 | 888 | 14 | A25G23C21T32 | A29G28C21T43 | A17G13C13T21 |
| A. baumannii | 29 | 1326 | 14 | A25G23C21T32 | A29G28C21T43 | A17G13C13T21 |
| A. baumannii | 35 | 1440 | 14 | A25G23C21T32 | A29G28C21T43 | A17G13C13T21 |
| A. baumannii | 41 | 1524 | 14 | A25G23C21T32 | A29G28C21T43 | A17G13C13T21 |
| A. baumannii | 46 | 1652 | 14 | A25G23C21T32 | A29G28C21T43 | A17G13C13T21 |
| A. baumannii | 47 | 1653 | 14 | A25G23C21T32 | A29G28C21T43 | A17G13C13T21 |
| A. baumannii | 48 | 1657 | 14 | A25G23C21T32 | A29G28C21T43 | A17G13C13T21 |
| A. baumannii | 57 | 1709 | 14 | A25G23C21T32 | A29G28C21T43 | A17G13C13T21 |
| A. baumannii | 61 | 1727 | 14 | A25G23C21T32 | A29G28C21T43 | A17G13C13T21 |
| A. baumannii | 63 | 1762 | 14 | A25G23C21T32 | A29G28C21T43 | A17G13C13T21 |
| A. baumannii | 67 | 1806 | 14 | A25G23C21T32 | A29G28C21T43 | A17G13C13T21 |
| A. baumannii | 75 | 1881 | 14 | A25G23C21T32 | A29G28C21T43 | A17G13C13T21 |
| A. baumannii | 77 | 1886 | 14 | A25G23C21T32 | A29G28C21T43 | A17G13C13T21 |
| A. baumannii | 1 | 649 | 46 | A25G23C21T32 | A29G28C21T43 | A17G13C13T21 |
| A. baumannii | 2 | 653 | 46 | A25G23C21T32 | A29G28C21T43 | A17G13C13T21 |
| A. baumannii | 39 | 1497 | 16 | A25G23C21T32 | A29G28C21T43 | A17G13C13T21 |
| A. baumannii | 24 | 1198 | 15 | A25G23C21T32 | A29G28C21T43 | A17G13C13T21 |
| A. baumannii | 28 | 1243 | 15 | A25G23C21T32 | A29G28C21T43 | A17G13C13T21 |
| A. baumannii | 43 | 1648 | 15 | A25G23C21T32 | A29G28C21T43 | A17G13C13T21 |
| A. baumannii | 62 | 1746 | 15 | A25G23C21T32 | A29G28C21T43 | A17G13C13T21 |
| A. baumannii | 4 | 689 | 15 | A25G23C21T32 | A29G28C21T43 | A17G13C13T21 |
| A. baumannii | 68 | 1822 | 3 | A25G23C21T32 | A29G28C21T43 | A17G13C13T21 |
| A. baumannii | 69 | 1823A | 3 | A25G23C21T32 | A29G28C21T43 | A17G13C13T21 |
| A. baumannii | 70 | 1823B | 3 | A25G23C21T32 | A29G28C21T43 | A17G13C13T21 |
| A. baumannii | 71 | 1826 | 3 | A25G23C21T32 | A29G28C21T43 | A17G13C13T21 |
| A. baumannii | 72 | 1860 | 3 | A25G23C21T32 | A29G28C21T43 | A17G13C13T21 |
| A. baumannii | 81 | 1924 | 3 | A25G23C21T32 | A29G28C21T43 | A17G13C13T21 |
| A. baumannii | 82 | 1929 | 3 | A25G23C21T32 | A29G28C21T43 | A17G13C13T21 |
| A. baumannii | 85 | 1966 | 3 | A25G23C21T32 | A29G28C21T43 | A17G13C13T21 |
| A. baumannii | 11 | 841 | 3 | A25G23C22T31 | A29G28C22T42 | A17G13C14T20 |
| A. baumannii | 32 | 1415 | 24 | A25G23C21T32 | A29G28C21T43 | A17G13C13T21 |
| A. baumannii | 45 | 1651 | 24 | A25G23C21T32 | A29G28C21T43 | A17G13C13T21 |
| A. baumannii | 54 | 1697 | 24 | A25G23C21T32 | A29G28C21T43 | A17G13C13T21 |
| A. baumannii | 58 | 1712 | 24 | A25G23C21T32 | A29G28C21T43 | A17G13C13T21 |
| A. baumannii | 60 | 1725 | 24 | A25G23C21T32 | A29G28C21T43 | A17G13C13T21 |
| A. baumannii | 66 | 1802 | 24 | A25G23C21T32 | A29G28C21T43 | A17G13C13T21 |
| A. baumannii | 76 | 1883 | 24 | A25G23C21T32 | A29G28C21T43 | A17G13C13T21 |
| A. baumannii | 78 | 1891 | 24 | A25G23C21T32 | A29G28C21T43 | A17G13C13T21 |
| A. baumannii | 79 | 1892 | 24 | A25G23C21T32 | A29G28C21T43 | A17G13C13T21 |
| A. baumannii | 83 | 1947 | 24 | A25G23C21T32 | A29G28C21T43 | A17G13C13T21 |
| A. baumannii | 84 | 1964 | 24 | A25G23C21T32 | A29G28C21T43 | A17G13C13T21 |
| A. baumannii | 53 | 1696 | 24 | A25G23C22T31 | A29G28C22T42 | A17G13C14T20 |
| A. baumannii | 36 | 1458 | 49 | A25G23C21T32 | A29G28C21T43 | A17G13C13T21 |
| A. baumannii | 59 | 1716 | 9 | A25G23C22T31 | A29G28C22T42 | A17G13C14T20 |
| A. baumannii | 9 | 805 | 30 | A25G23C22T31 | A29G28C22T42 | A17G13C14T20 |
| A. baumannii | 18 | 967 | 39 | A25G23C22T31 | A29G28C22T42 | A17G13C14T20 |
| A. baumannii | 30 | 1322 | 48 | A25G23C22T31 | A29G28C22T42 | A17G13C14T20 |
| A. baumannii | 26 | 1218 | 50 | A25G23C22T31 | A29G28C22T42 | A17G13C14T20 |
| A. sp. 13TU | 15 | 875 | A1 | A25G23C22T31 | A29G28C22T42 | A17G13C14T20 |
| A. sp. 13TU | 17 | 895 | A1 | A25G23C22T31 | A29G28C22T42 | A17G13C14T20 |
| A. sp. 3 | 12 | 853 | B7 | A25G22C22T32 | A30G29C22T40 | A17G13C14T20 |
| A. johnsonii | 25 | 1202 | NEW1 | A25G22C22T32 | A30G29C22T40 | A17G13C14T20 |
| A. sp. 2082 | 87 | 2082 | NEW2 | A25G22C22T32 | A31G28C22T40 | A17G13C14T20 |

TABLE 15B
Base Compositions Determined from A. baumannii DNA Samples Obtained from
Walter Reed Hospital and Amplified with Codon Analysis Primer Pairs Targeting the parC Gene

| | | | | | | PP No: 2848 |
|---|---|---|---|---|---|---|
| Species | Ibis# | Isolate | ST | PP No: 2846 parC | PP No: 2847 parC | parC |
| A. baumannii | 20 | 1082 | 1 | A33G26C29T33 | A29G28C26T31 | A16G14C15T15 |
| A. baumannii | 13 | 854 | 10 | A33G26C28T34 | A29G28C25T32 | A16G14C14T16 |
| A. baumannii | 22 | 1162 | 10 | A33G26C28T34 | A29G28C25T32 | A16G14C14T16 |
| A. baumannii | 27 | 1230 | 10 | A33G26C28T34 | A29G28C25T32 | A16G14C14T16 |
| A. baumannii | 31 | 1367 | 10 | A33G26C28T34 | A29G28C25T32 | A16G14C14T16 |
| A. baumannii | 37 | 1459 | 10 | A33G26C28T34 | A29G28C25T32 | A16G14C14T16 |
| A. baumannii | 55 | 1700 | 10 | A33G26C28T34 | A29G28C25T32 | A16G14C14T16 |
| A. baumannii | 64 | 1777 | 10 | A33G26C28T34 | A29G28C25T32 | A16G14C14T16 |
| A. baumannii | 73 | 1861 | 10 | A33G26C28T34 | A29G28C25T32 | A16G14C14T16 |
| A. baumannii | 74 | 1877 | 10 | A33G26C28T34 | A29G28C25T32 | A16G14C14T16 |
| A. baumannii | 86 | 1972 | 10 | A33G26C28T34 | A29G28C25T32 | A16G14C14T16 |
| A. baumannii | 3 | 684 | 11 | A33G26C28T34 | A29G28C25T32 | A16G14C14T16 |
| A. baumannii | 6 | 720 | 11 | A33G26C28T34 | A29G28C25T32 | A16G14C14T16 |
| A. baumannii | 7 | 726 | 11 | A33G26C28T34 | A29G28C25T32 | A16G14C14T16 |
| A. baumannii | 19 | 1079 | 11 | A33G26C28T34 | A29G28C25T32 | A16G14C14T16 |
| A. baumannii | 21 | 1123 | 11 | A33G26C28T34 | A29G28C25T32 | A16G14C14T16 |
| A. baumannii | 23 | 1188 | 11 | A33G26C28T34 | A29G28C25T32 | A16G14C14T16 |
| A. baumannii | 33 | 1417 | 11 | A33G26C28T34 | A29G28C25T32 | A16G14C14T16 |
| A. baumannii | 34 | 1431 | 11 | A33G26C28T34 | A29G28C25T32 | A16G14C14T16 |
| A. baumannii | 38 | 1496 | 11 | A33G26C28T34 | A29G28C25T32 | A16G14C14T16 |
| A. baumannii | 40 | 1523 | 11 | A33G26C28T34 | A29G28C25T32 | A16G14C14T16 |
| A. baumannii | 42 | 1640 | 11 | A33G26C28T34 | A29G28C25T32 | A16G14C14T16 |
| A. baumannii | 50 | 1666 | 11 | A33G26C28T34 | A29G28C25T32 | A16G14C14T16 |
| A. baumannii | 51 | 1668 | 11 | A33G26C28T34 | A29G28C25T32 | A16G14C14T16 |
| A. baumannii | 52 | 1695 | 11 | A33G26C28T34 | A29G28C25T32 | A16G14C14T16 |
| A. baumannii | 65 | 1781 | 11 | A33G26C28T34 | A29G28C25T32 | A16G14C14T16 |
| A. baumannii | 44 | 1649 | 12 | A33G26C28T34 | A29G28C25T32 | A16G14C14T16 |
| A. baumannii | 49A | 1658.1 | 12 | A33G26C28T34 | A29G28C25T32 | A16G14C14T16 |
| A. baumannii | 49B | 1658.2 | 12 | A33G26C28T34 | A29G28C25T32 | A16G14C14T16 |
| A. baumannii | 56 | 1707 | 12 | A33G26C28T34 | A29G28C25T32 | A16G14C14T16 |
| A. baumannii | 80 | 1893 | 12 | A33G26C28T34 | A29G28C25T32 | A16G14C14T16 |
| A. baumannii | 5 | 693 | 14 | A33G26C28T34 | A29G28C25T32 | A16G14C14T16 |
| A. baumannii | 8 | 749 | 14 | A33G26C28T34 | A29G28C25T32 | A16G14C14T16 |
| A. baumannii | 10 | 839 | 14 | A33G26C28T34 | A29G28C25T32 | A16G14C14T16 |
| A. baumannii | 14 | 865 | 14 | A33G26C28T34 | A29G28C25T32 | A16G14C14T16 |
| A. baumannii | 16 | 888 | 14 | A33G26C28T34 | A29G28C25T32 | A16G14C14T16 |
| A. baumannii | 29 | 1326 | 14 | A33G26C28T34 | A29G28C25T32 | A16G14C14T16 |
| A. baumannii | 35 | 1440 | 14 | A33G26C28T34 | A29G28C25T32 | A16G14C14T16 |
| A. baumannii | 41 | 1524 | 14 | A33G26C28T34 | A29G28C25T32 | A16G14C14T16 |
| A. baumannii | 46 | 1652 | 14 | A33G26C28T34 | A29G28C25T32 | A16G14C14T16 |
| A. baumannii | 47 | 1653 | 14 | A33G26C28T34 | A29G28C25T32 | A16G14C14T16 |
| A. baumannii | 48 | 1657 | 14 | A33G26C28T34 | A29G28C25T32 | A16G14C14T16 |
| A. baumannii | 57 | 1709 | 14 | A33G26C28T34 | A29G28C25T32 | A16G14C14T16 |
| A. baumannii | 61 | 1727 | 14 | A33G26C28T34 | A29G28C25T32 | A16G14C14T16 |
| A. baumannii | 63 | 1762 | 14 | A33G26C28T34 | A29G28C25T32 | A16G14C14T16 |
| A. baumannii | 67 | 1806 | 14 | A33G26C28T34 | A29G28C25T32 | A16G14C14T16 |
| A. baumannii | 75 | 1881 | 14 | A33G26C28T34 | A29G28C25T32 | A16G14C14T16 |
| A. baumannii | 77 | 1886 | 14 | A33G26C28T34 | A29G28C25T32 | A16G14C14T16 |
| A. baumannii | 1 | 649 | 46 | A33G26C28T34 | A29G28C25T32 | A16G14C14T16 |
| A. baumannii | 2 | 653 | 46 | A33G26C28T34 | A29G28C25T32 | A16G14C14T16 |
| A. baumannii | 39 | 1497 | 16 | A33G26C28T34 | A29G28C25T32 | A16G14C14T16 |
| A. baumannii | 24 | 1198 | 15 | A33G26C28T34 | A29G29C23T33 | A16G14C14T16 |
| A. baumannii | 28 | 1243 | 15 | A33G26C28T34 | A29G29C23T33 | A16G14C14T16 |
| A. baumannii | 43 | 1648 | 15 | A33G26C28T34 | A29G29C23T33 | A16G14C14T16 |
| A. baumannii | 62 | 1746 | 15 | A33G26C28T34 | A29G29C23T33 | A16G14C14T16 |
| A. baumannii | 4 | 689 | 15 | A34G25C29T33 | A30G27C26T31 | A16G14C15T15 |
| A. baumannii | 68 | 1822 | 3 | A33G26C28T34 | A29G28C25T32 | A16G14C14T16 |
| A. baumannii | 69 | 1823A | 3 | A33G26C28T34 | A29G28C25T32 | A16G14C14T16 |
| A. baumannii | 70 | 1823B | 3 | A33G26C28T34 | A29G28C25T32 | A16G14C14T16 |
| A. baumannii | 71 | 1826 | 3 | A33G26C28T34 | A29G28C25T32 | A16G14C14T16 |
| A. baumannii | 72 | 1860 | 3 | A33G26C28T34 | A29G28C25T32 | A16G14C14T16 |
| A. baumannii | 81 | 1924 | 3 | A33G26C28T34 | A29G28C25T32 | A16G14C14T16 |
| A. baumannii | 82 | 1929 | 3 | A33G26C28T34 | A29G28C25T32 | A16G14C14T16 |
| A. baumannii | 85 | 1966 | 3 | A33G26C28T34 | A29G28C25T32 | A16G14C14T16 |
| A. baumannii | 11 | 841 | 3 | A33G26C29T33 | A29G28C26T31 | A16G14C15T15 |
| A. baumannii | 32 | 1415 | 24 | A33G26C29T33 | A29G28C26T31 | A16G14C15T15 |
| A. baumannii | 45 | 1651 | 24 | A33G26C29T33 | A29G28C26T31 | A16G14C15T15 |
| A. baumannii | 54 | 1697 | 24 | A33G26C29T33 | A29G28C26T31 | A16G14C15T15 |
| A. baumannii | 58 | 1712 | 24 | A33G26C29T33 | A29G28C26T31 | A16G14C15T15 |
| A. baumannii | 60 | 1725 | 24 | A33G26C29T33 | A29G28C26T31 | A16G14C15T15 |
| A. baumannii | 66 | 1802 | 24 | A33G26C29T33 | A29G28C26T31 | A16G14C15T15 |
| A. baumannii | 76 | 1883 | 24 | A33G26C29T33 | A29G28C26T31 | A16G14C15T15 |
| A. baumannii | 78 | 1891 | 24 | A34G25C29T33 | A30G27C26T31 | A16G14C15T15 |
| A. baumannii | 79 | 1892 | 24 | A33G26C29T33 | A29G28C26T31 | A16G14C15T15 |
| A. baumannii | 83 | 1947 | 24 | A34G25C29T33 | A30G27C26T31 | A16G14C15T15 |
| A. baumannii | 84 | 1964 | 24 | A33G26C29T33 | A29G28C26T31 | A16G14C15T15 |
| A. baumannii | 53 | 1696 | 24 | A33G26C29T33 | A29G28C26T31 | A16G14C15T15 |
| A. baumannii | 36 | 1458 | 49 | A34G26C29T32 | A30G28C24T32 | A16G14C15T15 |
| A. baumannii | 59 | 1716 | 9 | A33G26C29T33 | A29G28C26T31 | A16G14C15T15 |
| A. baumannii | 9 | 805 | 30 | A33G26C29T33 | A29G28C26T31 | A16G14C15T15 |
| A. baumannii | 18 | 967 | 39 | A33G26C29T33 | A29G28C26T31 | A16G14C15T15 |
| A. baumannii | 30 | 1322 | 48 | A33G26C29T33 | A29G28C26T31 | A16G14C15T15 |
| A. baumannii | 26 | 1218 | 50 | A33G26C29T33 | A29G28C26T31 | A16G14C15T15 |
| A. sp. 13TU | 15 | 875 | A1 | A32G26C28T35 | A28G28C24T34 | A16G14C15T15 |
| A. sp. 13TU | 17 | 895 | A1 | A32G26C28T35 | A28G28C24T34 | A16G14C15T15 |
| A. sp. 3 | 12 | 853 | B7 | A29G26C27T39 | A26G32C21T35 | A16G14C15T15 |
| A. johnsonii | 25 | 1202 | NEW1 | A32G28C26T35 | A29G29C22T34 | A16G14C15T15 |
| A. sp. 2082 | 87 | 2082 | NEW2 | A33G27C26T35 | A31G28C20T35 | A16G14C15T15 |

TABLE 16A
Base Compositions Determined from A. baumannii DNA Samples Obtained from Northwestern
Medical Center and Amplified with Codon Analysis Primer Pairs Targeting the gyrA Gene

| | | | | | | PP No: 2854 |
|---|---|---|---|---|---|---|
| Species | Ibis# | Isolate | ST | PP No: 2852 gyrA | PP No: 2853 gyrA | gyrA |
| A. baumannii | 54 | 536 | 3 | A25G23C21T32 | A29G28C21T43 | A17G13C13T21 |
| A. baumannii | 87 | 665 | 3 | A25G23C21T32 | A29G28C21T43 | A17G13C13T21 |
| A. baumannii | 8 | 80 | 10 | A25G23C21T32 | A29G28C21T43 | A17G13C13T21 |
| A. baumannii | 9 | 91 | 10 | A25G23C21T32 | A29G28C21T43 | A17G13C13T21 |
| A. baumannii | 10 | 92 | 10 | A25G23C21T32 | A29G28C21T43 | A17G13C13T21 |
| A. baumannii | 11 | 131 | 10 | A25G23C21T32 | A29G28C21T43 | A17G13C13T21 |
| A. baumannii | 12 | 137 | 10 | A25G23C21T32 | A29G28C21T43 | A17G13C13T21 |
| A. baumannii | 21 | 218 | 10 | A25G23C21T32 | A29G28C21T43 | A17G13C13T21 |
| A. baumannii | 26 | 242 | 10 | A25G23C21T32 | A29G28C21T43 | A17G13C13T21 |
| A. baumannii | 94 | 678 | 10 | A25G23C21T32 | A29G28C21T43 | A17G13C13T21 |
| A. baumannii | 1 | 9 | 10 | A25G23C21T32 | A29G28C21T43 | A17G13C13T21 |
| A. baumannii | 2 | 13 | 10 | A25G23C21T32 | A29G28C21T43 | A17G13C13T21 |
| A. baumannii | 3 | 19 | 10 | A25G23C21T32 | A29G28C21T43 | A17G13C13T21 |
| A. baumannii | 4 | 24 | 10 | A25G23C21T32 | A29G28C21T43 | A17G13C13T21 |
| A. baumannii | 5 | 36 | 10 | A25G23C21T32 | A29G28C21T43 | A17G13C13T21 |
| A. baumannii | 6 | 39 | 10 | A25G23C21T32 | A29G28C21T43 | A17G13C13T21 |
| A. baumannii | 13 | 139 | 10 | A25G23C21T32 | A29G28C21T43 | A17G13C13T21 |
| A. baumannii | 15 | 165 | 10 | A25G23C21T32 | A29G28C21T43 | A17G13C13T21 |
| A. baumannii | 16 | 170 | 10 | A25G23C21T32 | A29G28C21T43 | A17G13C13T21 |
| A. baumannii | 17 | 186 | 10 | A25G23C21T32 | A29G28C21T43 | A17G13C13T21 |
| A. baumannii | 20 | 202 | 10 | A25G23C21T32 | A29G28C21T43 | A17G13C13T21 |
| A. baumannii | 22 | 221 | 10 | A25G23C21T32 | A29G28C21T43 | A17G13C13T21 |
| A. baumannii | 24 | 234 | 10 | A25G23C21T32 | A29G28C21T43 | A17G13C13T21 |
| A. baumannii | 25 | 239 | 10 | A25G23C21T32 | A29G28C21T43 | A17G13C13T21 |
| A. baumannii | 33 | 370 | 10 | A25G23C21T32 | A29G28C21T43 | A17G13C13T21 |
| A. baumannii | 34 | 389 | 10 | A25G23C21T32 | A29G28C21T43 | A17G13C13T21 |
| A. baumannii | 19 | 201 | 14 | A25G23C21T32 | A29G28C21T43 | A17G13C13T21 |
| A. baumannii | 27 | 257 | 51 | A25G23C21T32 | A29G28C21T43 | A17G13C13T21 |
| A. baumannii | 29 | 301 | 51 | A25G23C21T32 | A29G28C21T43 | A17G13C13T21 |
| A. baumannii | 31 | 354 | 51 | A25G23C21T32 | A29G28C21T43 | A17G13C13T21 |
| A. baumannii | 36 | 422 | 51 | A25G23C21T32 | A29G28C21T43 | A17G13C13T21 |
| A. baumannii | 37 | 424 | 51 | A25G23C21T32 | A29G28C21T43 | A17G13C13T21 |
| A. baumannii | 38 | 434 | 51 | A25G23C21T32 | A29G28C21T43 | A17G13C13T21 |
| A. baumannii | 39 | 473 | 51 | A25G23C21T32 | A29G28C21T43 | A17G13C13T21 |
| A. baumannii | 40 | 482 | 51 | A25G23C21T32 | A29G28C21T43 | A17G13C13T21 |
| A. baumannii | 44 | 512 | 51 | A25G23C21T32 | A29G28C21T43 | A17G13C13T21 |
| A. baumannii | 45 | 516 | 51 | A25G23C21T32 | A29G28C21T43 | A17G13C13T21 |
| A. baumannii | 47 | 522 | 51 | A25G23C21T32 | A29G28C21T43 | A17G13C13T21 |
| A. baumannii | 48 | 526 | 51 | A25G23C21T32 | A29G28C21T43 | A17G13C13T21 |
| A. baumannii | 50 | 528 | 51 | A25G23C21T32 | A29G28C21T43 | A17G13C13T21 |
| A. baumannii | 52 | 531 | 51 | A25G23C21T32 | A29G28C21T43 | A17G13C13T21 |
| A. baumannii | 53 | 533 | 51 | A25G23C21T32 | A29G28C21T43 | A17G13C13T21 |
| A. baumannii | 56 | 542 | 51 | A25G23C21T32 | A29G28C21T43 | A17G13C13T21 |
| A. baumannii | 59 | 550 | 51 | A25G23C21T32 | A29G28C21T43 | A17G13C13T21 |
| A. baumannii | 62 | 556 | 51 | A25G23C21T32 | A29G28C21T43 | A17G13C13T21 |
| A. baumannii | 64 | 557 | 51 | A25G23C21T32 | A29G28C21T43 | A17G13C13T21 |
| A. baumannii | 70 | 588 | 51 | A25G23C21T32 | A29G28C21T43 | A17G13C13T21 |
| A. baumannii | 73 | 603 | 51 | A25G23C21T32 | A29G28C21T43 | A17G13C13T21 |
| A. baumannii | 74 | 605 | 51 | A25G23C21T32 | A29G28C21T43 | A17G13C13T21 |
| A. baumannii | 75 | 606 | 51 | A25G23C21T32 | A29G28C21T43 | A17G13C13T21 |
| A. baumannii | 77 | 611 | 51 | A25G23C21T32 | A29G28C21T43 | A17G13C13T21 |
| A. baumannii | 79 | 622 | 51 | A25G23C21T32 | A29G28C21T43 | A17G13C13T21 |
| A. baumannii | 83 | 643 | 51 | A25G23C21T32 | A29G28C21T43 | A17G13C13T21 |
| A. baumannii | 85 | 653 | 51 | A25G23C21T32 | A29G28C21T43 | A17G13C13T21 |
| A. baumannii | 89 | 669 | 51 | A25G23C21T32 | A29G28C21T43 | A17G13C13T21 |
| A. baumannii | 93 | 674 | 51 | A25G23C21T32 | A29G28C21T43 | A17G13C13T21 |
| A. baumannii | 23 | 228 | 51 | A25G23C21T32 | A29G28C21T43 | A17G13C13T21 |
| A. baumannii | 32 | 369 | 52 | A25G23C21T32 | A29G28C21T43 | A17G13C13T21 |
| A. baumannii | 35 | 393 | 52 | A25G23C21T32 | A29G28C21T43 | A17G13C13T21 |
| A. baumannii | 30 | 339 | 53 | A25G23C21T32 | A29G28C21T43 | A17G13C13T21 |
| A. baumannii | 41 | 485 | 53 | A25G23C21T32 | A29G28C21T43 | A17G13C13T21 |
| A. baumannii | 42 | 493 | 53 | A25G23C21T32 | A29G28C21T43 | A17G13C13T21 |
| A. baumannii | 43 | 502 | 53 | A25G23C21T32 | A29G28C21T43 | A17G13C13T21 |
| A. baumannii | 46 | 520 | 53 | A25G23C21T32 | A29G28C21T43 | A17G13C13T21 |
| A. baumannii | 49 | 527 | 53 | A25G23C21T32 | A29G28C21T43 | A17G13C13T21 |
| A. baumannii | 51 | 529 | 53 | A25G23C21T32 | A29G28C21T43 | A17G13C13T21 |
| A. baumannii | 65 | 562 | 53 | A25G23C21T32 | A29G28C21T43 | A17G13C13T21 |
| A. baumannii | 68 | 579 | 53 | A25G23C21T32 | A29G28C21T43 | A17G13C13T21 |
| A. baumannii | 57 | 546 | 54 | A25G23C21T32 | A29G28C21T43 | A17G13C13T21 |
| A. baumannii | 58 | 548 | 54 | A25G23C21T32 | A29G28C21T43 | A17G13C13T21 |
| A. baumannii | 60 | 552 | 54 | A25G23C21T32 | A29G28C21T43 | A17G13C13T21 |
| A. baumannii | 61 | 555 | 54 | A25G23C21T32 | A29G28C21T43 | A17G13C13T21 |
| A. baumannii | 63 | 557 | 54 | A25G23C21T32 | A29G28C21T43 | A17G13C13T21 |
| A. baumannii | 66 | 570 | 54 | A25G23C21T32 | A29G28C21T43 | A17G13C13T21 |
| A. baumannii | 67 | 578 | 54 | A25G23C21T32 | A29G28C21T43 | A17G13C13T21 |
| A. baumannii | 69 | 584 | 54 | A25G23C21T32 | A29G28C21T43 | A17G13C13T21 |
| A. baumannii | 71 | 593 | 54 | A25G23C21T32 | A29G28C21T43 | A17G13C13T21 |
| A. baumannii | 72 | 602 | 54 | A25G23C21T32 | A29G28C21T43 | A17G13C13T21 |
| A. baumannii | 76 | 609 | 54 | A25G23C21T32 | A29G28C21T43 | A17G13C13T21 |
| A. baumannii | 78 | 621 | 54 | A25G23C21T32 | A29G28C21T43 | A17G13C13T21 |
| A. baumannii | 80 | 625 | 54 | A25G23C21T32 | A29G28C21T43 | A17G13C13T21 |
| A. baumannii | 81 | 628 | 54 | A25G23C21T32 | A29G28C21T43 | A17G13C13T21 |
| A. baumannii | 82 | 632 | 54 | A25G23C21T32 | A29G28C21T43 | A17G13C13T21 |
| A. baumannii | 84 | 649 | 54 | A25G23C21T32 | A29G28C21T43 | A17G13C13T21 |
| A. baumannii | 86 | 655 | 54 | A25G23C21T32 | A29G28C21T43 | A17G13C13T21 |
| A. baumannii | 88 | 668 | 54 | A25G23C21T32 | A29G28C21T43 | A17G13C13T21 |
| A. baumannii | 90 | 671 | 54 | A25G23C21T32 | A29G28C21T43 | A17G13C13T21 |
| A. baumannii | 91 | 672 | 54 | A25G23C21T32 | A29G28C21T43 | A17G13C13T21 |
| A. baumannii | 92 | 673 | 54 | A25G23C21T32 | A29G28C21T43 | A17G13C13T21 |
| A. baumannii | 18 | 196 | 55 | A25G23C22T31 | A29G28C21T43 | A17G13C13T21 |
| A. baumannii | 55 | 537 | 27 | A25G23C21T32 | A29G28C21T43 | A17G13C13T21 |
| A. baumannii | 28 | 263 | 27 | A25G23C22T31 | A29G28C22T42 | A17G13C14T20 |
| A. sp. 3 | 14 | 164 | B7 | A25G22C22T32 | A30G29C22T40 | A17G13C14T20 |
| mixture | 7 | 71 | — | ND | ND | A17G13C15T19 |

TABLE 16B
Base Compositions Determined from A. baumannii DNA Samples
Obtained from Northwestern Medical Center and Amplified with
Codon Analysis Primer Pairs Targeting the parC Gene

| Species | Ibis# | Isolate | ST | PP No: 2846 parC | PP No: 2847 parC | PP No: 2848 parC |
|---|---|---|---|---|---|---|
| A. baumannii | 54 | 536 | 3 | A33G26C28T34 | A29G28C25T32 | A16G14C14T16 |
| A. baumannii | 87 | 665 | 3 | A33G26C28T34 | A29G28C25T32 | A16G14C14T16 |
| A. baumannii | 8 | 80 | 10 | A33G26C28T34 | A29G28C25T32 | A16G14C14T16 |
| A. baumannii | 9 | 91 | 10 | A33G26C28T34 | A29G28C25T32 | A16G14C14T16 |
| A. baumannii | 10 | 92 | 10 | A33G26C28T34 | A29G28C25T32 | ND |
| A. baumannii | 11 | 131 | 10 | A33G26C28T34 | A29G28C25T32 | A16G14C14T16 |
| A. baumannii | 12 | 137 | 10 | A33G26C28T34 | A29G28C25T32 | A16G14C14T16 |
| A. baumannii | 21 | 218 | 10 | A33G26C28T34 | A29G28C25T32 | A16G14C14T16 |
| A. baumannii | 26 | 242 | 10 | A33G26C28T34 | A29G28C25T32 | A16G14C14T16 |
| A. baumannii | 94 | 678 | 10 | A33G26C28T34 | A29G28C25T32 | A16G14C14T16 |
| A. baumannii | 1 | 9 | 10 | A33G26C29T33 | A29G28C26T31 | A16G14C15T15 |
| A. baumannii | 2 | 13 | 10 | A33G26C29T33 | A29G28C26T31 | A16G14C15T15 |
| A. baumannii | 3 | 19 | 10 | A33G26C29T33 | A29G28C26T31 | A16G14C15T15 |
| A. baumannii | 4 | 24 | 10 | A33G26C29T33 | A29G28C26T31 | A16G14C15T15 |
| A. baumannii | 5 | 36 | 10 | A33G26C29T33 | A29G28C26T31 | A16G14C15T15 |
| A. baumannii | 6 | 39 | 10 | A33G26C29T33 | A29G28C26T31 | A16G14C15T15 |
| A. baumannii | 13 | 139 | 10 | A33G26C29T33 | A29G28C26T31 | A16G14C15T15 |
| A. baumannii | 15 | 165 | 10 | A33G26C29T33 | A29G28C26T31 | A16G14C15T15 |
| A. baumannii | 16 | 170 | 10 | A33G26C29T33 | A29G28C26T31 | A16G14C15T15 |
| A. baumannii | 17 | 186 | 10 | A33G26C29T33 | A29G28C26T31 | A16G14C15T15 |
| A. baumannii | 20 | 202 | 10 | A33G26C29T33 | A29G28C26T31 | A16G14C15T15 |
| A. baumannii | 22 | 221 | 10 | A33G26C29T33 | A29G28C26T31 | A16G14C15T15 |
| A. baumannii | 24 | 234 | 10 | A33G26C29T33 | A29G28C26T31 | A16G14C15T15 |
| A. baumannii | 25 | 239 | 10 | A33G26C29T33 | A29G28C26T31 | A16G14C15T15 |
| A. baumannii | 33 | 370 | 10 | A33G26C29T33 | A29G28C26T31 | A16G14C15T15 |
| A. baumannii | 34 | 389 | 10 | A33G26C29T33 | A29G28C26T31 | A16G14C15T15 |
| A. baumannii | 19 | 201 | 14 | A33G26C28T34 | A29G28C25T32 | A16G14C14T16 |
| A. baumannii | 27 | 257 | 51 | A33G26C28T34 | A29G28C25T32 | A16G14C14T16 |
| A. baumannii | 29 | 301 | 51 | A33G26C28T34 | A29G28C25T32 | A16G14C14T16 |
| A. baumannii | 31 | 354 | 51 | A33G26C28T34 | A29G28C25T32 | A16G14C14T16 |
| A. baumannii | 36 | 422 | 51 | A33G26C28T34 | A29G28C25T32 | A16G14C14T16 |
| A. baumannii | 37 | 424 | 51 | A33G26C28T34 | A29G28C25T32 | A16G14C14T16 |
| A. baumannii | 38 | 434 | 51 | A33G26C28T34 | A29G28C25T32 | A16G14C14T16 |
| A. baumannii | 39 | 473 | 51 | A33G26C28T34 | A29G28C25T32 | A16G14C14T16 |
| A. baumannii | 40 | 482 | 51 | A33G26C28T34 | A29G28C25T32 | A16G14C14T16 |
| A. baumannii | 44 | 512 | 51 | A33G26C28T34 | A29G28C25T32 | A16G14C14T16 |
| A. baumannii | 45 | 516 | 51 | A33G26C28T34 | A29G28C25T32 | A16G14C14T16 |
| A. baumannii | 47 | 522 | 51 | A33G26C28T34 | A29G28C25T32 | A16G14C14T16 |
| A. baumannii | 48 | 526 | 51 | A33G26C28T34 | A29G28C25T32 | A16G14C14T16 |
| A. baumannii | 50 | 528 | 51 | A33G26C28T34 | A29G28C25T32 | A16G14C14T16 |
| A. baumannii | 52 | 531 | 51 | A33G26C28T34 | A29G28C25T32 | A16G14C14T16 |
| A. baumannii | 53 | 533 | 51 | A33G26C28T34 | A29G28C25T32 | A16G14C14T16 |
| A. baumannii | 56 | 542 | 51 | A33G26C28T34 | A29G28C25T32 | A16G14C14T16 |
| A. baumannii | 59 | 550 | 51 | A33G26C28T34 | A29G28C25T32 | A16G14C14T16 |
| A. baumannii | 62 | 556 | 51 | A33G26C28T34 | A29G28C25T32 | A16G14C14T16 |
| A. baumannii | 64 | 557 | 51 | A33G26C28T34 | A29G28C25T32 | A16G14C14T16 |
| A. baumannii | 70 | 588 | 51 | A33G26C28T34 | A29G28C25T32 | A16G14C14T16 |
| A. baumannii | 73 | 603 | 51 | A33G26C28T34 | A29G28C25T32 | A16G14C14T16 |
| A. baumannii | 74 | 605 | 51 | A33G26C28T34 | A29G28C25T32 | A16G14C14T16 |
| A. baumannii | 75 | 606 | 51 | A33G26C28T34 | A29G28C25T32 | A16G14C14T16 |
| A. baumannii | 77 | 611 | 51 | A33G26C28T34 | A29G28C25T32 | A16G14C14T16 |
| A. baumannii | 79 | 622 | 51 | A33G26C28T34 | A29G28C25T32 | A16G14C14T16 |
| A. baumannii | 83 | 643 | 51 | A33G26C28T34 | A29G28C25T32 | A16G14C14T16 |
| A. baumannii | 85 | 653 | 51 | A33G26C28T34 | A29G28C25T32 | A16G14C14T16 |
| A. baumannii | 89 | 669 | 51 | A33G26C28T34 | A29G28C25T32 | A16G14C14T16 |
| A. baumannii | 93 | 674 | 51 | A33G26C28T34 | A29G28C25T32 | A16G14C14T16 |
| A. baumannii | 23 | 228 | 51 | A34G25C29T33 | A30G27C26T31 | A16G14C15T15 |
| A. baumannii | 32 | 369 | 52 | A34G25C28T34 | A30G27C25T32 | A16G14C14T16 |
| A. baumannii | 35 | 393 | 52 | A34G25C28T34 | A30G27C25T32 | A16G14C14T16 |
| A. baumannii | 30 | 339 | 53 | A34G25C29T33 | A30G27C26T31 | A16G14C15T15 |
| A. baumannii | 41 | 485 | 53 | A34G25C29T33 | A30G27C26T31 | A16G14C15T15 |
| A. baumannii | 42 | 493 | 53 | A34G25C29T33 | A30G27C26T31 | A16G14C15T15 |
| A. baumannii | 43 | 502 | 53 | A34G25C29T33 | A30G27C26T31 | A16G14C15T15 |
| A. baumannii | 46 | 520 | 53 | A34G25C29T33 | A30G27C26T31 | A16G14C15T15 |
| A. baumannii | 49 | 527 | 53 | A34G25C29T33 | A30G27C26T31 | A16G14C15T15 |
| A. baumannii | 51 | 529 | 53 | A34G25C29T33 | A30G27C26T31 | A16G14C15T15 |
| A. baumannii | 65 | 562 | 53 | A34G25C29T33 | A30G27C26T31 | A16G14C15T15 |
| A. baumannii | 68 | 579 | 53 | A34G25C29T33 | A30G27C26T31 | A16G14C15T15 |
| A. baumannii | 57 | 546 | 54 | A33G26C28T34 | A29G28C25T32 | A16G14C14T16 |
| A. baumannii | 58 | 548 | 54 | A33G26C28T34 | A29G28C25T32 | A16G14C14T16 |
| A. baumannii | 60 | 552 | 54 | A33G26C28T34 | A29G28C25T32 | A16G14C14T16 |
| A. baumannii | 61 | 555 | 54 | A33G26C28T34 | A29G28C25T32 | A16G14C14T16 |
| A. baumannii | 63 | 557 | 54 | A33G26C28T34 | A29G28C25T32 | A16G14C14T16 |
| A. baumannii | 66 | 570 | 54 | A33G26C28T34 | A29G28C25T32 | A16G14C14T16 |
| A. baumannii | 67 | 578 | 54 | A33G26C28T34 | A29G28C25T32 | A16G14C14T16 |
| A. baumannii | 69 | 584 | 54 | A33G26C28T34 | A29G28C25T32 | A16G14C14T16 |
| A. baumannii | 71 | 593 | 54 | A33G26C28T34 | A29G28C25T32 | A16G14C14T16 |
| A. baumannii | 72 | 602 | 54 | A33G26C28T34 | A29G28C25T32 | A16G14C14T16 |
| A. baumannii | 76 | 609 | 54 | A33G26C28T34 | A29G28C25T32 | A16G14C14T16 |
| A. baumannii | 78 | 621 | 54 | A33G26C28T34 | A29G28C25T32 | A16G14C14T16 |
| A. baumannii | 80 | 625 | 54 | A33G26C28T34 | A29G28C25T32 | A16G14C14T16 |
| A. baumannii | 81 | 628 | 54 | A33G26C28T34 | A29G28C25T32 | A16G14C14T16 |
| A. baumannii | 82 | 632 | 54 | A33G26C28T34 | A29G28C25T32 | A16G14C14T16 |
| A. baumannii | 84 | 649 | 54 | A33G26C28T34 | A29G28C25T32 | A16G14C14T16 |
| A. baumannii | 86 | 655 | 54 | A33G26C28T34 | A29G28C25T32 | A16G14C14T16 |
| A. baumannii | 88 | 668 | 54 | A33G26C28T34 | A29G28C25T32 | A16G14C14T16 |
| A. baumannii | 90 | 671 | 54 | A33G26C28T34 | A29G28C25T32 | A16G14C14T16 |
| A. baumannii | 91 | 672 | 54 | A33G26C28T34 | A29G28C25T32 | A16G14C14T16 |
| A. baumannii | 92 | 673 | 54 | A33G26C28T34 | A29G28C25T32 | A16G14C14T16 |
| A. baumannii | 18 | 196 | 55 | A33G27C28T33 | A29G28C25T31 | A15G14C15T16 |
| A. baumannii | 55 | 537 | 27 | A33G26C29T33 | A29G28C26T31 | A16G14C15T15 |
| A. baumannii | 28 | 263 | 27 | A33G26C29T33 | A29G28C26T31 | A16G14C15T15 |
| A. sp. 3 | 14 | 164 | B7 | A35G25C29T32 | A30G28C17T39 | A16G14C15T15 |
| mixture | 7 | 71 | — | ND | ND | A17G14C15T14 |

TABLE 17A
Base Compositions Determined from A. baumannii DNA Samples Obtained from
Walter Reed Hospital and Amplified with Speciating Primer Pair No. 2922
and Triangulation Genotyping Analysis Primer Pair Nos. 1151 and 1156

| Species | Ibis# | Isolate | ST | PP No: 2922 efp | PP No: 1151 trpE | PP No: 1156 Adk |
|---|---|---|---|---|---|---|
| A. baumannii | 20 | 1082 | 1 | A45G34C25T43 | A44G35C21T42 | A44G32C26T38 |
| A. baumannii | 13 | 854 | 10 | A45G34C25T43 | A44G35C21T42 | A44G32C26T38 |
| A. baumannii | 22 | 1162 | 10 | A45G34C25T43 | A44G35C21T42 | A44G32C26T38 |
| A. baumannii | 27 | 1230 | 10 | A45G34C25T43 | A44G35C21T42 | A44G32C26T38 |
| A. baumannii | 31 | 1367 | 10 | A45G34C25T43 | A44G35C21T42 | A44G32C26T38 |
| A. baumannii | 37 | 1459 | 10 | A45G34C25T43 | A44G35C21T42 | A44G32C26T38 |
| A. baumannii | 55 | 1700 | 10 | A45G34C25T43 | A44G35C21T42 | A44G32C26T38 |
| A. baumannii | 64 | 1777 | 10 | A45G34C25T43 | A44G35C21T42 | A44G32C26T38 |
| A. baumannii | 73 | 1861 | 10 | A45G34C25T43 | A44G35C21T42 | A44G32C26T38 |
| A. baumannii | 74 | 1877 | 10 | A45G34C25T43 | A44G35C21T42 | A44G32C26T38 |
| A. baumannii | 86 | 1972 | 10 | A45G34C25T43 | A44G35C21T42 | A44G32C26T38 |
| A. baumannii | 3 | 684 | 11 | A45G34C25T43 | A44G35C21T42 | A44G32C26T38 |
| A. baumannii | 6 | 720 | 11 | A45G34C25T43 | A44G35C21T42 | A44G32C26T38 |
| A. baumannii | 7 | 726 | 11 | A45G34C25T43 | A44G35C21T42 | A44G32C26T38 |
| A. baumannii | 19 | 1079 | 11 | A45G34C25T43 | A44G35C21T42 | A44G32C26T38 |
| A. baumannii | 21 | 1123 | 11 | A45G34C25T43 | A44G35C21T42 | A44G32C26T38 |
| A. baumannii | 23 | 1188 | 11 | A45G34C25T43 | A44G35C21T42 | A44G32C26T38 |
| A. baumannii | 33 | 1417 | 11 | A45G34C25T43 | A44G35C21T42 | A44G32C26T38 |
| A. baumannii | 34 | 1431 | 11 | A45G34C25T43 | A44G35C21T42 | A44G32C26T38 |
| A. baumannii | 38 | 1496 | 11 | A45G34C25T43 | A44G35C21T42 | A44G32C26T38 |
| A. baumannii | 40 | 1523 | 11 | A45G34C25T43 | A44G35C21T42 | A44G32C26T38 |
| A. baumannii | 42 | 1640 | 11 | A45G34C25T43 | A44G35C21T42 | A44G32C26T38 |
| A. baumannii | 50 | 1666 | 11 | A45G34C25T43 | A44G35C21T42 | A44G32C26T38 |
| A. baumannii | 51 | 1668 | 11 | A45G34C25T43 | A44G35C21T42 | A44G32C26T38 |
| A. baumannii | 52 | 1695 | 11 | A45G34C25T43 | A44G35C21T42 | A44G32C26T38 |
| A. baumannii | 65 | 1781 | 11 | A45G34C25T43 | A44G35C21T42 | A44G32C26T38 |
| A. baumannii | 44 | 1649 | 12 | A45G34C25T43 | A44G35C21T42 | A44G32C26T38 |
| A. baumannii | 49A | 1658.1 | 12 | A45G34C25T43 | A44G35C21T42 | A44G32C26T38 |
| A. baumannii | 49B | 1658.2 | 12 | A45G34C25T43 | A44G35C21T42 | A44G32C26T38 |
| A. baumannii | 56 | 1707 | 12 | A45G34C25T43 | A44G35C21T42 | A44G32C26T38 |
| A. baumannii | 80 | 1893 | 12 | A45G34C25T43 | A44G35C21T42 | A44G32C26T38 |
| A. baumannii | 5 | 693 | 14 | A44G35C25T43 | A44G35C22T41 | A44G32C27T37 |
| A. baumannii | 8 | 749 | 14 | A44G35C25T43 | A44G35C22T41 | A44G32C27T37 |
| A. baumannii | 10 | 839 | 14 | A44G35C25T43 | A44G35C22T41 | A44G32C27T37 |
| A. baumannii | 14 | 865 | 14 | A44G35C25T43 | A44G35C22T41 | A44G32C27T37 |
| A. baumannii | 16 | 888 | 14 | A44G35C25T43 | A44G35C22T41 | A44G32C27T37 |
| A. baumannii | 29 | 1326 | 14 | A44G35C25T43 | A44G35C22T41 | A44G32C27T37 |
| A. baumannii | 35 | 1440 | 14 | A44G35C25T43 | ND | A44G32C27T37 |
| A. baumannii | 41 | 1524 | 14 | A44G35C25T43 | A44G35C22T41 | A44G32C27T37 |
| A. baumannii | 46 | 1652 | 14 | A44G35C25T43 | A44G35C22T41 | A44G32C27T37 |
| A. baumannii | 47 | 1653 | 14 | A44G35C25T43 | A44G35C22T41 | A44G32C27T37 |
| A. baumannii | 48 | 1657 | 14 | A44G35C25T43 | A44G35C22T41 | A44G32C27T37 |
| A. baumannii | 57 | 1709 | 14 | A44G35C25T43 | A44G35C22T41 | A44G32C27T37 |
| A. baumannii | 61 | 1727 | 14 | A44G35C25T43 | A44G35C22T41 | A44G32C27T37 |
| A. baumannii | 63 | 1762 | 14 | A44G35C25T43 | A44G35C22T41 | A44G32C27T37 |
| A. baumannii | 67 | 1806 | 14 | A44G35C25T43 | A44G35C22T41 | A44G32C27T37 |
| A. baumannii | 75 | 1881 | 14 | A44G35C25T43 | A44G35C22T41 | A44G32C27T37 |
| A. baumannii | 77 | 1886 | 14 | A44G35C25T43 | A44G35C22T41 | A44G32C27T37 |
| A. baumannii | 1 | 649 | 46 | A44G35C25T43 | A44G35C22T41 | A44G32C26T38 |
| A. baumannii | 2 | 653 | 46 | A44G35C25T43 | A44G35C22T41 | A44G32C26T38 |
| A. baumannii | 39 | 1497 | 16 | A44G35C25T43 | A44G35C22T41 | A44G32C27T37 |
| A. baumannii | 24 | 1198 | 15 | A44G35C25T43 | A44G35C22T41 | A44G32C26T38 |
| A. baumannii | 28 | 1243 | 15 | A44G35C25T43 | A44G35C22T41 | A44G32C26T38 |
| A. baumannii | 43 | 1648 | 15 | A44G35C25T43 | A44G35C22T41 | A44G32C26T38 |
| A. baumannii | 62 | 1746 | 15 | A44G35C25T43 | A44G35C22T41 | A44G32C26T38 |
| A. baumannii | 4 | 689 | 15 | A44G35C25T43 | A44G35C22T41 | A44G32C26T38 |
| A. baumannii | 68 | 1822 | 3 | A44G35C24T44 | A44G35C22T41 | A44G32C26T38 |
| A. baumannii | 69 | 1823A | 3 | A44G35C24T44 | A44G35C22T41 | A44G32C26T38 |
| A. baumannii | 70 | 1823B | 3 | A44G35C24T44 | A44G35C22T41 | A44G32C26T38 |
| A. baumannii | 71 | 1826 | 3 | A44G35C24T44 | A44G35C22T41 | A44G32C26T38 |
| A. baumannii | 72 | 1860 | 3 | A44G35C24T44 | A44G35C22T41 | A44G32C26T38 |
| A. baumannii | 81 | 1924 | 3 | A44G35C24T44 | A44G35C22T41 | A44G32C26T38 |
| A. baumannii | 82 | 1929 | 3 | A44G35C24T44 | A44G35C22T41 | A44G32C26T38 |
| A. baumannii | 85 | 1966 | 3 | A44G35C24T44 | A44G35C22T41 | A44G32C26T38 |
| A. baumannii | 11 | 841 | 3 | A44G35C24T44 | A44G35C22T41 | A44G32C26T38 |
| A. baumannii | 32 | 1415 | 24 | A44G35C25T43 | A43G36C20T43 | A44G32C27T37 |
| A. baumannii | 45 | 1651 | 24 | A44G35C25T43 | A43G36C20T43 | A44G32C27T37 |
| A. baumannii | 54 | 1697 | 24 | A44G35C25T43 | A43G36C20T43 | A44G32C27T37 |
| A. baumannii | 58 | 1712 | 24 | A44G35C25T43 | A43G36C20T43 | A44G32C27T37 |
| A. baumannii | 60 | 1725 | 24 | A44G35C25T43 | A43G36C20T43 | A44G32C27T37 |
| A. baumannii | 66 | 1802 | 24 | A44G35C25T43 | A43G36C20T43 | A44G32C27T37 |
| A. baumannii | 76 | 1883 | 24 | ND | A43G36C20T43 | A44G32C27T37 |
| A. baumannii | 78 | 1891 | 24 | A44G35C25T43 | A43G36C20T43 | A44G32C27T37 |
| A. baumannii | 79 | 1892 | 24 | A44G35C25T43 | A43G36C20T43 | A44G32C27T37 |
| A. baumannii | 83 | 1947 | 24 | A44G35C25T43 | A43G36C20T43 | A44G32C27T37 |
| A. baumannii | 84 | 1964 | 24 | A44G35C25T43 | A43G36C20T43 | A44G32C27T37 |
| A. baumannii | 53 | 1696 | 24 | A44G35C25T43 | A43G36C20T43 | A44G32C27T37 |
| A. baumannii | 36 | 1458 | 49 | A44G35C25T43 | A44G35C22T41 | A44G32C27T37 |
| A. baumannii | 59 | 1716 | 9 | A44G35C25T43 | A44G35C21T42 | A44G32C26T38 |
| A. baumannii | 9 | 805 | 30 | A44G35C25T43 | A44G35C19T44 | A44G32C27T37 |
| A. baumannii | 18 | 967 | 39 | A45G34C25T43 | A44G35C22T41 | A44G32C26T38 |
| A. baumannii | 30 | 1322 | 48 | A44G35C25T43 | A43G36C20T43 | A44G32C27T37 |
| A. baumannii | 26 | 1218 | 50 | A44G35C25T43 | A44G35C21T42 | A44G32C26T38 |
| A. sp. 13TU | 15 | 875 | A1 | A47G33C24T43 | A46G32C20T44 | A44G33C27T36 |
| A. sp. 13TU | 17 | 895 | A1 | A47G33C24T43 | A46G32C20T44 | A44G33C27T36 |
| A. sp. 3 | 12 | 853 | B7 | A46G35C24T42 | A42G34C20T46 | A43G33C24T40 |
| A. johnsonii | 25 | 1202 | NEW1 | A46G35C23T43 | A42G35C21T44 | A43G33C23T41 |
| A. sp. 2082 | 87 | 2082 | NEW2 | A46G36C22T43 | A42G32C20T48 | A42G34C23T41 |

TABLE 17B
Base Compositions Determined from A. baumannii DNA Samples Obtained from Walter Reed Hospital
and Amplified with Triangulation Genotyping Analysis Primer Pair Nos. 1158 and 1160 and 1165

| Species | Ibis# | Isolate | ST | PP No: 1158 mutY | PP No: 1160 mutY | PP No: 1165 fumC |
|---|---|---|---|---|---|---|
| A. baumannii | 20 | 1082 | 1 | A27G21C25T22 | A32G35C29T33 | A40G33C30T36 |
| A. baumannii | 13 | 854 | 10 | A27G21C26T21 | A32G35C28T34 | A40G33C30T36 |
| A. baumannii | 22 | 1162 | 10 | A27G21C26T21 | A32G35C28T34 | A40G33C30T36 |
| A. baumannii | 27 | 1230 | 10 | A27G21C26T21 | A32G35C28T34 | A40G33C30T36 |
| A. baumannii | 31 | 1367 | 10 | A27G21C26T21 | A32G35C28T34 | A40G33C30T36 |
| A. baumannii | 37 | 1459 | 10 | A27G21C26T21 | A32G35C28T34 | A40G33C30T36 |
| A. baumannii | 55 | 1700 | 10 | A27G21C26T21 | A32G35C28T34 | A40G33C30T36 |
| A. baumannii | 64 | 1777 | 10 | A27G21C26T21 | A32G35C28T34 | A40G33C30T36 |
| A. baumannii | 73 | 1861 | 10 | A27G21C26T21 | A32G35C28T34 | A40G33C30T36 |
| A. baumannii | 74 | 1877 | 10 | A27G21C26T21 | A32G35C28T34 | A40G33C30T36 |
| A. baumannii | 86 | 1972 | 10 | A27G21C26T21 | A32G35C28T34 | A40G33C30T36 |
| A. baumannii | 3 | 684 | 11 | A27G21C25T22 | A32G34C28T35 | A40G33C30T36 |
| A. baumannii | 6 | 720 | 11 | A27G21C25T22 | A32G34C28T35 | A40G33C30T36 |
| A. baumannii | 7 | 726 | 11 | A27G21C25T22 | A32G34C28T35 | A40G33C30T36 |
| A. baumannii | 19 | 1079 | 11 | A27G21C25T22 | A32G34C28T35 | A40G33C30T36 |
| A. baumannii | 21 | 1123 | 11 | A27G21C25T22 | A32G34C28T35 | A40G33C30T36 |
| A. baumannii | 23 | 1188 | 11 | A27G21C25T22 | A32G34C28T35 | A40G33C30T36 |
| A. baumannii | 33 | 1417 | 11 | A27G21C25T22 | A32G34C28T35 | A40G33C30T36 |
| A. baumannii | 34 | 1431 | 11 | A27G21C25T22 | A32G34C28T35 | A40G33C30T36 |
| A. baumannii | 38 | 1496 | 11 | A27G21C25T22 | A32G34C28T35 | A40G33C30T36 |
| A. baumannii | 40 | 1523 | 11 | A27G21C25T22 | A32G34C28T35 | A40G33C30T36 |
| A. baumannii | 42 | 1640 | 11 | A27G21C25T22 | A32G34C28T35 | A40G33C30T36 |
| A. baumannii | 50 | 1666 | 11 | A27G21C25T22 | A32G34C28T35 | A40G33C30T36 |
| A. baumannii | 51 | 1668 | 11 | A27G21C25T22 | A32G34C28T35 | A40G33C30T36 |
| A. baumannii | 52 | 1695 | 11 | A27G21C25T22 | A32G34C28T35 | A40G33C30T36 |
| A. baumannii | 65 | 1781 | 11 | A27G21C25T22 | A32G34C28T35 | A40G33C30T36 |
| A. baumannii | 44 | 1649 | 12 | A27G21C26T21 | A32G34C29T34 | A40G33C30T36 |
| A. baumannii | 49A | 1658.1 | 12 | A27G21C26T21 | A32G34C29T34 | A40G33C30T36 |
| A. baumannii | 49B | 1658.2 | 12 | A27G21C26T21 | A32G34C29T34 | A40G33C30T36 |
| A. baumannii | 56 | 1707 | 12 | A27G21C26T21 | A32G34C29T34 | A40G33C30T36 |
| A. baumannii | 80 | 1893 | 12 | A27G21C26T21 | A32G34C29T34 | A40G33C30T36 |
| A. baumannii | 5 | 693 | 14 | A27G21C25T22 | A31G36C28T34 | A40G33C29T37 |
| A. baumannii | 8 | 749 | 14 | A27G21C25T22 | A31G36C28T34 | A40G33C29T37 |
| A. baumannii | 10 | 839 | 14 | A27G21C25T22 | A31G36C28T34 | A40G33C29T37 |
| A. baumannii | 14 | 865 | 14 | A27G21C25T22 | A31G36C28T34 | A40G33C29T37 |
| A. baumannii | 16 | 888 | 14 | A27G21C25T22 | A31G36C28T34 | A40G33C29T37 |
| A. baumannii | 29 | 1326 | 14 | A27G21C25T22 | A31G36C28T34 | A40G33C29T37 |
| A. baumannii | 35 | 1440 | 14 | A27G21C25T22 | A31G36C28T34 | A40G33C29T37 |
| A. baumannii | 41 | 1524 | 14 | A27G21C25T22 | A31G36C28T34 | A40G33C29T37 |
| A. baumannii | 46 | 1652 | 14 | A27G21C25T22 | A31G36C28T34 | A40G33C29T37 |
| A. baumannii | 47 | 1653 | 14 | A27G21C25T22 | A31G36C28T34 | A40G33C29T37 |
| A. baumannii | 48 | 1657 | 14 | A27G21C25T22 | A31G36C28T34 | A40G33C29T37 |
| A. baumannii | 57 | 1709 | 14 | A27G21C25T22 | A31G36C28T34 | A40G33C29T37 |
| A. baumannii | 61 | 1727 | 14 | A27G21C25T22 | A31G36C28T34 | A40G33C29T37 |
| A. baumannii | 63 | 1762 | 14 | A27G21C25T22 | A31G36C28T34 | A40G33C29T37 |
| A. baumannii | 67 | 1806 | 14 | A27G21C25T22 | A31G36C28T34 | A40G33C29T37 |
| A. baumannii | 75 | 1881 | 14 | A27G21C25T22 | A31G36C28T34 | A40G33C29T37 |
| A. baumannii | 77 | 1886 | 14 | A27G21C25T22 | A31G36C28T34 | A40G33C29T37 |
| A. baumannii | 1 | 649 | 46 | A29G19C26T21 | A31G35C29T34 | A40G33C29T37 |
| A. baumannii | 2 | 653 | 46 | A29G19C26T21 | A31G35C29T34 | A40G33C29T37 |
| A. baumannii | 39 | 1497 | 16 | A29G19C26T21 | A31G35C29T34 | A40G34C29T36 |
| A. baumannii | 24 | 1198 | 15 | A29G19C26T21 | A31G35C29T34 | A40G33C29T37 |
| A. baumannii | 28 | 1243 | 15 | A29G19C26T21 | A31G35C29T34 | A40G33C29T37 |
| A. baumannii | 43 | 1648 | 15 | A29G19C26T21 | A31G35C29T34 | A40G33C29T37 |
| A. baumannii | 62 | 1746 | 15 | A29G19C26T21 | A31G35C29T34 | A40G33C29T37 |
| A. baumannii | 4 | 689 | 15 | A29G19C26T21 | A31G35C29T34 | A40G33C29T37 |
| A. baumannii | 68 | 1822 | 3 | A27G20C27T21 | A32G35C28T34 | A40G33C30T36 |
| A. baumannii | 69 | 1823A | 3 | A27G20C27T21 | A32G35C28T34 | A40G33C30T36 |
| A. baumannii | 70 | 1823B | 3 | A27G20C27T21 | A32G35C28T34 | A40G33C30T36 |
| A. baumannii | 71 | 1826 | 3 | A27G20C27T21 | A32G35C28T34 | A40G33C30T36 |
| A. baumannii | 72 | 1860 | 3 | A27G20C27T21 | A32G35C28T34 | A40G33C30T36 |
| A. baumannii | 81 | 1924 | 3 | A27G20C27T21 | A32G35C28T34 | A40G33C30T36 |
| A. baumannii | 82 | 1929 | 3 | A27G20C27T21 | A32G35C28T34 | A40G33C30T36 |
| A. baumannii | 85 | 1966 | 3 | A27G20C27T21 | A32G35C28T34 | A40G33C30T36 |
| A. baumannii | 11 | 841 | 3 | A27G20C27T21 | A32G35C28T34 | A40G33C30T36 |
| A. baumannii | 32 | 1415 | 24 | A27G21C26T21 | A32G35C28T34 | A40G33C30T36 |
| A. baumannii | 45 | 1651 | 24 | A27G21C26T21 | A32G35C28T34 | A40G33C30T36 |
| A. baumannii | 54 | 1697 | 24 | A27G21C26T21 | A32G35C28T34 | A40G33C30T36 |
| A. baumannii | 58 | 1712 | 24 | A27G21C26T21 | A32G35C28T34 | A40G33C30T36 |
| A. baumannii | 60 | 1725 | 24 | A27G21C26T21 | A32G35C28T34 | A40G33C30T36 |
| A. baumannii | 66 | 1802 | 24 | A27G21C26T21 | A32G35C28T34 | A40G33C30T36 |
| A. baumannii | 76 | 1883 | 24 | A27G21C26T21 | A32G35C28T34 | A40G33C30T36 |
| A. baumannii | 78 | 1891 | 24 | A27G21C26T21 | A32G35C28T34 | A40G33C30T36 |
| A. baumannii | 79 | 1892 | 24 | A27G21C26T21 | A32G35C28T34 | A40G33C30T36 |
| A. baumannii | 83 | 1947 | 24 | A27G21C26T21 | A32G35C28T34 | A40G33C30T36 |
| A. baumannii | 84 | 1964 | 24 | A27G21C26T21 | A32G35C28T34 | A40G33C30T36 |
| A. baumannii | 53 | 1696 | 24 | A27G21C26T21 | A32G35C28T34 | A40G33C30T36 |
| A. baumannii | 36 | 1458 | 49 | A27G20C27T21 | A32G35C28T34 | A40G33C30T36 |
| A. baumannii | 59 | 1716 | 9 | A27G21C25T22 | A32G35C28T34 | A39G33C30T37 |
| A. baumannii | 9 | 805 | 30 | A27G21C25T22 | A32G35C28T34 | A39G33C30T37 |
| A. baumannii | 18 | 967 | 39 | A27G21C26T21 | A32G35C28T34 | A39G33C30T37 |
| A. baumannii | 30 | 1322 | 48 | A28G21C24T22 | A32G35C29T33 | A40G33C30T36 |
| A. baumannii | 26 | 1218 | 50 | A27G21C25T22 | A31G36C28T34 | A40G33C29T37 |
| A. sp. 13TU | 15 | 875 | A1 | A27G21C25T22 | A30G36C26T37 | A41G34C28T36 |
| A. sp. 13TU | 17 | 895 | A1 | A27G21C25T22 | A30G36C26T37 | A41G34C28T36 |
| A. sp. 3 | 12 | 853 | B7 | A26G23C23T23 | A30G36C27T36 | A39G37C26T37 |
| A. johnsonii | 25 | 1202 | NEW1 | A25G23C24T23 | A30G35C30T34 | A38G37C26T38 |
| A. sp. 2082 | 87 | 2082 | NEW2 | A26G22C24T23 | A31G35C28T35 | A42G34C27T36 |

TABLE 17C
Base Compositions Determined from A. baumannii DNA Samples Obtained from Walter Reed Hospital
and Amplified with Triangulation Genotyping Analysis Primer Pair Nos. 1167 and 1170 and 1171

| Species | Ibis# | Isolate | ST | PP No: 1167 fumC | PP No: 1170 fumC | PP No: 1171 ppa |
|---|---|---|---|---|---|---|
| A. baumannii | 20 | 1082 | 1 | A41G34C34T38 | A38G27C21T50 | A35G37C33T44 |
| A. baumannii | 13 | 854 | 10 | A41G34C34T38 | A38G27C21T50 | A35G37C33T44 |
| A. baumannii | 22 | 1162 | 10 | A41G34C34T38 | A38G27C21T50 | A35G37C33T44 |
| A. baumannii | 27 | 1230 | 10 | A41G34C34T38 | A38G27C21T50 | A35G37C33T44 |
| A. baumannii | 31 | 1367 | 10 | A41G34C34T38 | A38G27C21T50 | A35G37C33T44 |
| A. baumannii | 37 | 1459 | 10 | A41G34C34T38 | A38G27C21T50 | A35G37C33T44 |
| A. baumannii | 55 | 1700 | 10 | A41G34C34T38 | A38G27C21T50 | A35G37C33T44 |
| A. baumannii | 64 | 1777 | 10 | A41G34C34T38 | A38G27C21T50 | A35G37C33T44 |
| A. baumannii | 73 | 1861 | 10 | A41G34C34T38 | A38G27C21T50 | A35G37C33T44 |
| A. baumannii | 74 | 1877 | 10 | A41G34C34T38 | A38G27C21T50 | A35G37C33T44 |
| A. baumannii | 86 | 1972 | 10 | A41G34C34T38 | A38G27C21T50 | A35G37C33T44 |
| A. baumannii | 3 | 684 | 11 | A41G34C34T38 | A38G27C21T50 | A35G37C33T44 |
| A. baumannii | 6 | 720 | 11 | A41G34C34T38 | A38G27C21T50 | A35G37C33T44 |
| A. baumannii | 7 | 726 | 11 | A41G34C34T38 | A38G27C21T50 | A35G37C33T44 |
| A. baumannii | 19 | 1079 | 11 | A41G34C34T38 | A38G27C21T50 | A35G37C33T44 |
| A. baumannii | 21 | 1123 | 11 | A41G34C34T38 | A38G27C21T50 | A35G37C33T44 |
| A. baumannii | 23 | 1188 | 11 | A41G34C34T38 | A38G27C21T50 | A35G37C33T44 |
| A. baumannii | 33 | 1417 | 11 | A41G34C34T38 | A38G27C21T50 | A35G37C33T44 |
| A. baumannii | 34 | 1431 | 11 | A41G34C34T38 | A38G27C21T50 | A35G37C33T44 |
| A. baumannii | 38 | 1496 | 11 | A41G34C34T38 | A38G27C21T50 | A35G37C33T44 |
| A. baumannii | 40 | 1523 | 11 | A41G34C34T38 | A38G27C21T50 | A35G37C33T44 |
| A. baumannii | 42 | 1640 | 11 | A41G34C34T38 | A38G27C21T50 | A35G37C33T44 |
| A. baumannii | 50 | 1666 | 11 | A41G34C34T38 | A38G27C21T50 | A35G37C33T44 |
| A. baumannii | 51 | 1668 | 11 | A41G34C34T38 | A38G27C21T50 | A35G37C33T44 |
| A. baumannii | 52 | 1695 | 11 | A41G34C34T38 | A38G27C21T50 | A35G37C33T44 |
| A. baumannii | 65 | 1781 | 11 | A41G34C34T38 | A38G27C21T50 | A35G37C33T44 |
| A. baumannii | 44 | 1649 | 12 | A41G34C34T38 | A38G27C21T50 | A35G37C33T44 |
| A. baumannii | 49A | 1658.1 | 12 | A41G34C34T38 | A38G27C21T50 | A35G37C33T44 |
| A. baumannii | 49B | 1658.2 | 12 | A41G34C34T38 | A38G27C21T50 | A35G37C33T44 |
| A. baumannii | 56 | 1707 | 12 | A41G34C34T38 | A38G27C21T50 | A35G37C33T44 |
| A. baumannii | 80 | 1893 | 12 | A41G34C34T38 | A38G27C21T50 | A35G37C33T44 |
| A. baumannii | 5 | 693 | 14 | A40G35C34T38 | A38G27C21T50 | A35G37C30T47 |
| A. baumannii | 8 | 749 | 14 | A40G35C34T38 | A38G27C21T50 | A35G37C30T47 |
| A. baumannii | 10 | 839 | 14 | A40G35C34T38 | A38G27C21T50 | A35G37C30T47 |
| A. baumannii | 14 | 865 | 14 | A40G35C34T38 | A38G27C21T50 | A35G37C30T47 |
| A. baumannii | 16 | 888 | 14 | A40G35C34T38 | A38G27C21T50 | A35G37C30T47 |
| A. baumannii | 29 | 1326 | 14 | A40G35C34T38 | A38G27C21T50 | A35G37C30T47 |
| A. baumannii | 35 | 1440 | 14 | A40G35C34T38 | A38G27C21T50 | A35G37C30T47 |
| A. baumannii | 41 | 1524 | 14 | A40G35C34T38 | A38G27C21T50 | A35G37C30T47 |
| A. baumannii | 46 | 1652 | 14 | A40G35C34T38 | A38G27C21T50 | A35G37C30T47 |
| A. baumannii | 47 | 1653 | 14 | A40G35C34T38 | A38G27C21T50 | A35G37C30T47 |
| A. baumannii | 48 | 1657 | 14 | A40G35C34T38 | A38G27C21T50 | A35G37C30T47 |
| A. baumannii | 57 | 1709 | 14 | A40G35C34T38 | A38G27C21T50 | A35G37C30T47 |
| A. baumannii | 61 | 1727 | 14 | A40G35C34T38 | A38G27C21T50 | A35G37C30T47 |
| A. baumannii | 63 | 1762 | 14 | A40G35C34T38 | A38G27C21T50 | A35G37C30T47 |
| A. baumannii | 67 | 1806 | 14 | A40G35C34T38 | A38G27C21T50 | A35G37C30T47 |
| A. baumannii | 75 | 1881 | 14 | A40G35C34T38 | A38G27C21T50 | A35G37C30T47 |
| A. baumannii | 77 | 1886 | 14 | A40G35C34T38 | A38G27C21T50 | A35G37C30T47 |
| A. baumannii | 1 | 649 | 46 | A41G35C32T39 | A37G28C20T51 | A35G37C32T45 |
| A. baumannii | 2 | 653 | 46 | A41G35C32T39 | A37G28C20T51 | A35G37C32T45 |
| A. baumannii | 39 | 1497 | 16 | A41G35C32T39 | A37G28C20T51 | A35G37C30T47 |
| A. baumannii | 24 | 1198 | 15 | A41G35C32T39 | A37G28C20T51 | A35G37C30T47 |
| A. baumannii | 28 | 1243 | 15 | A41G35C32T39 | A37G28C20T51 | A35G37C30T47 |
| A. baumannii | 43 | 1648 | 15 | A41G35C32T39 | A37G28C20T51 | A35G37C30T47 |
| A. baumannii | 62 | 1746 | 15 | A41G35C32T39 | A37G28C20T51 | A35G37C30T47 |
| A. baumannii | 4 | 689 | 15 | A41G35C32T39 | A37G28C20T51 | A35G37C30T47 |
| A. baumannii | 68 | 1822 | 3 | A41G34C35T37 | A38G27C20T51 | A35G37C31T46 |
| A. baumannii | 69 | 1823A | 3 | A41G34C35T37 | A38G27C20T51 | A35G37C31T46 |
| A. baumannii | 70 | 1823B | 3 | A41G34C35T37 | A38G27C20T51 | A35G37C31T46 |
| A. baumannii | 71 | 1826 | 3 | A41G34C35T37 | A38G27C20T51 | A35G37C31T46 |
| A. baumannii | 72 | 1860 | 3 | A41G34C35T37 | A38G27C20T51 | A35G37C31T46 |
| A. baumannii | 81 | 1924 | 3 | A41G34C35T37 | A38G27C20T51 | A35G37C31T46 |
| A. baumannii | 82 | 1929 | 3 | A41G34C35T37 | A38G27C20T51 | A35G37C31T46 |
| A. baumannii | 85 | 1966 | 3 | A41G34C35T37 | A38G27C20T51 | A35G37C31T46 |
| A. baumannii | 11 | 841 | 3 | A41G34C35T37 | A38G27C20T51 | A35G37C31T46 |
| A. baumannii | 32 | 1415 | 24 | A40G35C34T38 | A39G26C22T49 | A35G37C33T44 |
| A. baumannii | 45 | 1651 | 24 | A40G35C34T38 | A39G26C22T49 | A35G37C33T44 |
| A. baumannii | 54 | 1697 | 24 | A40G35C34T38 | A39G26C22T49 | A35G37C33T44 |
| A. baumannii | 58 | 1712 | 24 | A40G35C34T38 | A39G26C22T49 | A35G37C33T44 |
| A. baumannii | 60 | 1725 | 24 | A40G35C34T38 | A39G26C22T49 | A35G37C33T44 |
| A. baumannii | 66 | 1802 | 24 | A40G35C34T38 | A39G26C22T49 | A35G37C33T44 |
| A. baumannii | 76 | 1883 | 24 | A40G35C34T38 | A39G26C22T49 | A35G37C33T44 |
| A. baumannii | 78 | 1891 | 24 | A40G35C34T38 | A39G26C22T49 | A35G37C33T44 |
| A. baumannii | 79 | 1892 | 24 | A40G35C34T38 | A39G26C22T49 | A35G37C33T44 |
| A. baumannii | 83 | 1947 | 24 | A40G35C34T38 | A39G26C22T49 | A35G37C33T44 |
| A. baumannii | 84 | 1964 | 24 | A40G35C34T38 | A39G26C22T49 | A35G37C33T44 |
| A. baumannii | 53 | 1696 | 24 | A40G35C34T38 | A39G26C22T49 | A35G37C33T44 |
| A. baumannii | 36 | 1458 | 49 | A40G35C34T38 | A39G26C22T49 | A35G37C30T47 |
| A. baumannii | 59 | 1716 | 9 | A40G35C32T40 | A38G27C20T51 | A36G35C31T47 |
| A. baumannii | 9 | 805 | 30 | A40G35C32T40 | A38G27C21T50 | A35G36C29T49 |
| A. baumannii | 18 | 967 | 39 | A40G35C33T39 | A38G27C20T51 | A35G37C30T47 |
| A. baumannii | 30 | 1322 | 48 | A40G35C35T37 | A38G27C21T50 | A35G37C30T47 |
| A. baumannii | 26 | 1218 | 50 | A40G35C34T38 | A38G27C21T50 | A35G37C33T44 |
| A. sp. 13TU | 15 | 875 | A1 | A41G39C31T36 | A37G26C24T49 | A34G38C31T46 |
| A. sp. 13TU | 17 | 895 | A1 | A41G39C31T36 | A37G26C24T49 | A34G38C31T46 |
| A. sp. 3 | 12 | 853 | B7 | A43G37C30T37 | A36G27C24T49 | A34G37C31T47 |
| A. johnsonii | 25 | 1202 | NEW1 | A42G38C31T36 | A40G27C19T50 | A35G37C32T45 |
| A. sp. 2082 | 87 | 2082 | NEW2 | A43G37C32T35 | A37G26C21T52 | A35G38C31T45 |

TABLE 18A
Base Compositions Determined from A. baumannii DNA Samples Obtained from
Northwestern Medical Center and Amplified with Speciating Primer Pair No.
2922 and Triangulation Genotyping Analysis Primer Pair Nos. 1151 and 1156

| Species | Ibis# | Isolate | ST | PP No: 2922 efp | PP No: 1151 trpE | PP No: 1156 adk |
|---|---|---|---|---|---|---|
| A. baumannii | 54 | 536 | 3 | A44G35C24T44 | A44G35C22T41 | A44G32C26T38 |
| A. baumannii | 87 | 665 | 3 | A44G35C24T44 | A44G35C22T41 | A44G32C26T38 |
| A. baumannii | 8 | 80 | 10 | A45G34C25T43 | A44G35C21T42 | A44G32C26T38 |
| A. baumannii | 9 | 91 | 10 | A45G34C25T43 | A44G35C21T42 | A44G32C26T38 |
| A. baumannii | 10 | 92 | 10 | A45G34C25T43 | A44G35C21T42 | A44G32C26T38 |
| A. baumannii | 11 | 131 | 10 | A45G34C25T43 | A44G35C21T42 | A44G32C26T38 |
| A. baumannii | 12 | 137 | 10 | A45G34C25T43 | A44G35C21T42 | A44G32C26T38 |
| A. baumannii | 21 | 218 | 10 | A45G34C25T43 | A44G35C21T42 | A44G32C26T38 |
| A. baumannii | 26 | 242 | 10 | A45G34C25T43 | A44G35C21T42 | A44G32C26T38 |
| A. baumannii | 94 | 678 | 10 | A45G34C25T43 | A44G35C21T42 | A44G32C26T38 |
| A. baumannii | 1 | 9 | 10 | A45G34C25T43 | A44G35C21T42 | A44G32C26T38 |
| A. baumannii | 2 | 13 | 10 | A45G34C25T43 | A44G35C21T42 | A44G32C26T38 |
| A. baumannii | 3 | 19 | 10 | A45G34C25T43 | A44G35C21T42 | A44G32C26T38 |
| A. baumannii | 4 | 24 | 10 | A45G34C25T43 | A44G35C21T42 | A44G32C26T38 |
| A. baumannii | 5 | 36 | 10 | A45G34C25T43 | A44G35C21T42 | A44G32C26T38 |
| A. baumannii | 6 | 39 | 10 | A45G34C25T43 | A44G35C21T42 | A44G32C26T38 |
| A. baumannii | 13 | 139 | 10 | A45G34C25T43 | A44G35C21T42 | A44G32C26T38 |
| A. baumannii | 15 | 165 | 10 | A45G34C25T43 | A44G35C21T42 | A44G32C26T38 |
| A. baumannii | 16 | 170 | 10 | A45G34C25T43 | A44G35C21T42 | A44G32C26T38 |
| A. baumannii | 17 | 186 | 10 | A45G34C25T43 | A44G35C21T42 | A44G32C26T38 |
| A. baumannii | 20 | 202 | 10 | A45G34C25T43 | A44G35C21T42 | A44G32C26T38 |
| A. baumannii | 22 | 221 | 10 | A45G34C25T43 | A44G35C21T42 | A44G32C26T38 |
| A. baumannii | 24 | 234 | 10 | A45G34C25T43 | A44G35C21T42 | A44G32C26T38 |
| A. baumannii | 25 | 239 | 10 | A45G34C25T43 | A44G35C21T42 | A44G32C26T38 |
| A. baumannii | 33 | 370 | 10 | A45G34C25T43 | A44G35C21T42 | A44G32C26T38 |
| A. baumannii | 34 | 389 | 10 | A45G34C25T43 | A44G35C21T42 | A44G32C26T38 |
| A. baumannii | 19 | 201 | 14 | A44G35C25T43 | A44G35C22T41 | A44G32C27T37 |
| A. baumannii | 27 | 257 | 51 | A44G35C25T43 | A43G36C20T43 | A44G32C26T38 |
| A. baumannii | 29 | 301 | 51 | A44G35C25T43 | A43G36C20T43 | A44G32C26T38 |
| A. baumannii | 31 | 354 | 51 | A44G35C25T43 | A43G36C20T43 | A44G32C26T38 |
| A. baumannii | 36 | 422 | 51 | A44G35C25T43 | A43G36C20T43 | A44G32C26T38 |
| A. baumannii | 37 | 424 | 51 | A44G35C25T43 | A43G36C20T43 | A44G32C26T38 |
| A. baumannii | 38 | 434 | 51 | A44G35C25T43 | A43G36C20T43 | A44G32C26T38 |
| A. baumannii | 39 | 473 | 51 | A44G35C25T43 | A43G36C20T43 | A44G32C26T38 |
| A. baumannii | 40 | 482 | 51 | A44G35C25T43 | A43G36C20T43 | A44G32C26T38 |
| A. baumannii | 44 | 512 | 51 | A44G35C25T43 | A43G36C20T43 | A44G32C26T38 |
| A. baumannii | 45 | 516 | 51 | A44G35C25T43 | A43G36C20T43 | A44G32C26T38 |
| A. baumannii | 47 | 522 | 51 | A44G35C25T43 | A43G36C20T43 | A44G32C26T38 |
| A. baumannii | 48 | 526 | 51 | A44G35C25T43 | A43G36C20T43 | A44G32C26T38 |
| A. baumannii | 50 | 528 | 51 | A44G35C25T43 | A43G36C20T43 | A44G32C26T38 |
| A. baumannii | 52 | 531 | 51 | A44G35C25T43 | A43G36C20T43 | A44G32C26T38 |
| A. baumannii | 53 | 533 | 51 | A44G35C25T43 | A43G36C20T43 | A44G32C26T38 |
| A. baumannii | 56 | 542 | 51 | A44G35C25T43 | A43G36C20T43 | A44G32C26T38 |
| A. baumannii | 59 | 550 | 51 | A44G35C25T43 | A43G36C20T43 | A44G32C26T38 |
| A. baumannii | 62 | 556 | 51 | A44G35C25T43 | A43G36C20T43 | A44G32C26T38 |
| A. baumannii | 64 | 557 | 51 | A44G35C25T43 | A43G36C20T43 | A44G32C26T38 |
| A. baumannii | 70 | 588 | 51 | A44G35C25T43 | A43G36C20T43 | A44G32C26T38 |
| A. baumannii | 73 | 603 | 51 | A44G35C25T43 | A43G36C20T43 | A44G32C26T38 |
| A. baumannii | 74 | 605 | 51 | A44G35C25T43 | A43G36C20T43 | A44G32C26T38 |
| A. baumannii | 75 | 606 | 51 | A44G35C25T43 | A43G36C20T43 | A44G32C26T38 |
| A. baumannii | 77 | 611 | 51 | A44G35C25T43 | A43G36C20T43 | A44G32C26T38 |
| A. baumannii | 79 | 622 | 51 | A44G35C25T43 | A43G36C20T43 | A44G32C26T38 |
| A. baumannii | 83 | 643 | 51 | A44G35C25T43 | A43G36C20T43 | A44G32C26T38 |
| A. baumannii | 85 | 653 | 51 | A44G35C25T43 | A43G36C20T43 | A44G32C26T38 |
| A. baumannii | 89 | 669 | 51 | A44G35C25T43 | A43G36C20T43 | A44G32C26T38 |
| A. baumannii | 93 | 674 | 51 | A44G35C25T43 | A43G36C20T43 | A44G32C26T38 |
| A. baumannii | 23 | 228 | 51 | A44G35C25T43 | A43G36C20T43 | A44G32C26T38 |
| A. baumannii | 32 | 369 | 52 | A44G35C25T43 | A43G36C20T43 | A44G32C26T38 |
| A. baumannii | 35 | 393 | 52 | A44G35C25T43 | A43G36C20T43 | A44G32C26T38 |
| A. baumannii | 30 | 339 | 53 | A44G35C25T43 | A44G35C19T44 | A44G32C27T37 |
| A. baumannii | 41 | 485 | 53 | A44G35C25T43 | A44G35C19T44 | A44G32C27T37 |
| A. baumannii | 42 | 493 | 53 | A44G35C25T43 | A44G35C19T44 | A44G32C27T37 |
| A. baumannii | 43 | 502 | 53 | A44G35C25T43 | A44G35C19T44 | A44G32C27T37 |
| A. baumannii | 46 | 520 | 53 | A44G35C25T43 | A44G35C19T44 | A44G32C27T37 |
| A. baumannii | 49 | 527 | 53 | A44G35C25T43 | A44G35C19T44 | A44G32C27T37 |
| A. baumannii | 51 | 529 | 53 | A44G35C25T43 | A44G35C19T44 | A44G32C27T37 |
| A. baumannii | 65 | 562 | 53 | A44G35C25T43 | A44G35C19T44 | A44G32C27T37 |
| A. baumannii | 68 | 579 | 53 | A44G35C25T43 | A44G35C19T44 | A44G32C27T37 |
| A. baumannii | 57 | 546 | 54 | A44G35C25T43 | A44G35C20T43 | A44G32C26T38 |
| A. baumannii | 58 | 548 | 54 | A44G35C25T43 | A44G35C20T43 | A44G32C26T38 |
| A. baumannii | 60 | 552 | 54 | A44G35C25T43 | A44G35C20T43 | A44G32C26T38 |
| A. baumannii | 61 | 555 | 54 | A44G35C25T43 | A44G35C20T43 | A44G32C26T38 |
| A. baumannii | 63 | 557 | 54 | A44G35C25T43 | A44G35C20T43 | A44G32C26T38 |
| A. baumannii | 66 | 570 | 54 | A44G35C25T43 | A44G35C20T43 | A44G32C26T38 |
| A. baumannii | 67 | 578 | 54 | A44G35C25T43 | A44G35C20T43 | A44G32C26T38 |
| A. baumannii | 69 | 584 | 54 | A44G35C25T43 | A44G35C20T43 | A44G32C26T38 |
| A. baumannii | 71 | 593 | 54 | A44G35C25T43 | A44G35C20T43 | A44G32C26T38 |
| A. baumannii | 72 | 602 | 54 | A44G35C25T43 | A44G35C20T43 | A44G32C26T38 |
| A. baumannii | 76 | 609 | 54 | A44G35C25T43 | A44G35C20T43 | A44G32C26T38 |
| A. baumannii | 78 | 621 | 54 | A44G35C25T43 | A44G35C20T43 | A44G32C26T38 |
| A. baumannii | 80 | 625 | 54 | A44G35C25T43 | A44G35C20T43 | A44G32C26T38 |
| A. baumannii | 81 | 628 | 54 | A44G35C25T43 | A44G35C20T43 | A44G32C26T38 |
| A. baumannii | 82 | 632 | 54 | A44G35C25T43 | A44G35C20T43 | A44G32C26T38 |
| A. baumannii | 84 | 649 | 54 | A44G35C25T43 | A44G35C20T43 | A44G32C26T38 |
| A. baumannii | 86 | 655 | 54 | A44G35C25T43 | A44G35C20T43 | A44G32C26T38 |
| A. baumannii | 88 | 668 | 54 | A44G35C25T43 | A44G35C20T43 | A44G32C26T38 |
| A. baumannii | 90 | 671 | 54 | A44G35C25T43 | A44G35C20T43 | A44G32C26T38 |
| A. baumannii | 91 | 672 | 54 | A44G35C25T43 | A44G35C20T43 | A44G32C26T38 |
| A. baumannii | 92 | 673 | 54 | A44G35C25T43 | A44G35C20T43 | A44G32C26T38 |
| A. baumannii | 18 | 196 | 55 | A44G35C25T43 | A44G35C20T43 | A44G32C27T37 |
| A. baumannii | 55 | 537 | 27 | A44G35C25T43 | A44G35C19T44 | A44G32C27T37 |
| A. baumannii | 28 | 263 | 27 | A44G35C25T43 | A44G35C19T44 | A44G32C27T37 |
| A. sp. 3 | 14 | 164 | B7 | A46G35C24T42 | A42G34C20T46 | A43G33C24T40 |
| mixture | 7 | 71 | ? | mixture | ND | ND |

TABLE 18B
Base Compositions Determined from A. baumannii DNA Samples Obtained
from Northwestern Medical Center and Amplified with Triangulation
Genotyping Analysis Primer Pair Nos. 1158, 1160 and 1165

| Species | Ibis# | Isolate | ST | PP No: 1158 mutY | PP No: 1160 mutY | PP No: 1165 fumC |
|---|---|---|---|---|---|---|
| A. baumannii | 54 | 536 | 3 | A27G20C27T21 | A32G35C28T34 | A40G33C30T36 |
| A. baumannii | 87 | 665 | 3 | A27G20C27T21 | A32G35C28T34 | A40G33C30T36 |
| A. baumannii | 8 | 80 | 10 | A27G21C26T21 | A32G35C28T34 | A40G33C30T36 |
| A. baumannii | 9 | 91 | 10 | A27G21C26T21 | A32G35C28T34 | A40G33C30T36 |
| A. baumannii | 10 | 92 | 10 | A27G21C26T21 | A32G35C28T34 | A40G33C30T36 |
| A. baumannii | 11 | 131 | 10 | A27G21C26T21 | A32G35C28T34 | A40G33C30T36 |
| A. baumannii | 12 | 137 | 10 | A27G21C26T21 | A32G35C28T34 | A40G33C30T36 |
| A. baumannii | 21 | 218 | 10 | A27G21C26T21 | A32G35C28T34 | A40G33C30T36 |
| A. baumannii | 26 | 242 | 10 | A27G21C26T21 | A32G35C28T34 | A40G33C30T36 |
| A. baumannii | 94 | 678 | 10 | A27G21C26T21 | A32G35C28T34 | A40G33C30T36 |
| A. baumannii | 1 | 9 | 10 | A27G21C26T21 | A32G35C28T34 | A40G33C30T36 |
| A. baumannii | 2 | 13 | 10 | A27G21C26T21 | A32G35C28T34 | A40G33C30T36 |
| A. baumannii | 3 | 19 | 10 | A27G21C26T21 | A32G35C28T34 | A40G33C30T36 |
| A. baumannii | 4 | 24 | 10 | A27G21C26T21 | A32G35C28T34 | A40G33C30T36 |
| A. baumannii | 5 | 36 | 10 | A27G21C26T21 | A32G35C28T34 | A40G33C30T36 |
| A. baumannii | 6 | 39 | 10 | A27G21C26T21 | A32G35C28T34 | A40G33C30T36 |
| A. baumannii | 13 | 139 | 10 | A27G21C26T21 | A32G35C28T34 | A40G33C30T36 |
| A. baumannii | 15 | 165 | 10 | A27G21C26T21 | A32G35C28T34 | A40G33C30T36 |
| A. baumannii | 16 | 170 | 10 | A27G21C26T21 | A32G35C28T34 | A40G33C30T36 |
| A. baumannii | 17 | 186 | 10 | A27G21C26T21 | A32G35C28T34 | A40G33C30T36 |
| A. baumannii | 20 | 202 | 10 | A27G21C26T21 | A32G35C28T34 | A40G33C30T36 |
| A. baumannii | 22 | 221 | 10 | A27G21C26T21 | A32G35C28T34 | A40G33C30T36 |
| A. baumannii | 24 | 234 | 10 | A27G21C26T21 | A32G35C28T34 | A40G33C30T36 |
| A. baumannii | 25 | 239 | 10 | A27G21C26T21 | A32G35C28T34 | A40G33C30T36 |
| A. baumannii | 33 | 370 | 10 | A27G21C26T21 | A32G35C28T34 | A40G33C30T36 |
| A. baumannii | 34 | 389 | 10 | A27G21C26T21 | A32G35C28T34 | A40G33C30T36 |
| A. baumannii | 19 | 201 | 14 | A27G21C25T22 | A31G36C28T34 | A40G33C29T37 |
| A. baumannii | 27 | 257 | 51 | A27G21C25T22 | A32G35C28T34 | A40G33C29T37 |
| A. baumannii | 29 | 301 | 51 | A27G21C25T22 | A32G35C28T34 | A40G33C29T37 |
| A. baumannii | 31 | 354 | 51 | A27G21C25T22 | A32G35C28T34 | A40G33C29T37 |
| A. baumannii | 36 | 422 | 51 | A27G21C25T22 | A32G35C28T34 | A40G33C29T37 |
| A. baumannii | 37 | 424 | 51 | A27G21C25T22 | A32G35C28T34 | A40G33C29T37 |
| A. baumannii | 38 | 434 | 51 | A27G21C25T22 | A32G35C28T34 | A40G33C29T37 |
| A. baumannii | 39 | 473 | 51 | A27G21C25T22 | A32G35C28T34 | A40G33C29T37 |
| A. baumannii | 40 | 482 | 51 | A27G21C25T22 | A32G35C28T34 | A40G33C29T37 |
| A. baumannii | 44 | 512 | 51 | A27G21C25T22 | A32G35C28T34 | A40G33C29T37 |
| A. baumannii | 45 | 516 | 51 | A27G21C25T22 | A32G35C28T34 | A40G33C29T37 |
| A. baumannii | 47 | 522 | 51 | A27G21C25T22 | A32G35C28T34 | A40G33C29T37 |
| A. baumannii | 48 | 526 | 51 | A27G21C25T22 | A32G35C28T34 | A40G33C29T37 |
| A. baumannii | 50 | 528 | 51 | A27G21C25T22 | A32G35C28T34 | A40G33C29T37 |
| A. baumannii | 52 | 531 | 51 | A27G21C25T22 | A32G35C28T34 | A40G33C29T37 |
| A. baumannii | 53 | 533 | 51 | A27G21C25T22 | A32G35C28T34 | A40G33C29T37 |
| A. baumannii | 56 | 542 | 51 | A27G21C25T22 | A32G35C28T34 | A40G33C29T37 |
| A. baumannii | 59 | 550 | 51 | A27G21C25T22 | A32G35C28T34 | A40G33C29T37 |
| A. baumannii | 62 | 556 | 51 | A27G21C25T22 | A32G35C28T34 | A40G33C29T37 |
| A. baumannii | 64 | 557 | 51 | A27G21C25T22 | A32G35C28T34 | A40G33C29T37 |
| A. baumannii | 70 | 588 | 51 | A27G21C25T22 | A32G35C28T34 | A40G33C29T37 |
| A. baumannii | 73 | 603 | 51 | A27G21C25T22 | A32G35C28T34 | A40G33C29T37 |
| A. baumannii | 74 | 605 | 51 | A27G21C25T22 | A32G35C28T34 | A40G33C29T37 |
| A. baumannii | 75 | 606 | 51 | A27G21C25T22 | A32G35C28T34 | A40G33C29T37 |
| A. baumannii | 77 | 611 | 51 | A27G21C25T22 | A32G35C28T34 | A40G33C29T37 |
| A. baumannii | 79 | 622 | 51 | A27G21C25T22 | A32G35C28T34 | A40G33C29T37 |
| A. baumannii | 83 | 643 | 51 | A27G21C25T22 | A32G35C28T34 | A40G33C29T37 |
| A. baumannii | 85 | 653 | 51 | A27G21C25T22 | A32G35C28T34 | A40G33C29T37 |
| A. baumannii | 89 | 669 | 51 | A27G21C25T22 | A32G35C28T34 | A40G33C29T37 |
| A. baumannii | 93 | 674 | 51 | A27G21C25T22 | A32G35C28T34 | A40G33C29T37 |
| A. baumannii | 23 | 228 | 51 | A27G21C25T22 | A32G35C28T34 | A40G33C29T37 |
| A. baumannii | 32 | 369 | 52 | A27G21C25T22 | A32G35C28T34 | A40G33C29T37 |
| A. baumannii | 35 | 393 | 52 | A27G21C25T22 | A32G35C28T34 | A40G33C29T37 |
| A. baumannii | 30 | 339 | 53 | A28G20C26T21 | A32G34C29T34 | A40G33C30T36 |
| A. baumannii | 41 | 485 | 53 | A28G20C26T21 | A32G34C29T34 | A40G33C30T36 |
| A. baumannii | 42 | 493 | 53 | A28G20C26T21 | A32G34C29T34 | A40G33C30T36 |
| A. baumannii | 43 | 502 | 53 | A28G20C26T21 | A32G34C29T34 | A40G33C30T36 |
| A. baumannii | 46 | 520 | 53 | A28G20C26T21 | A32G34C29T34 | A40G33C30T36 |
| A. baumannii | 49 | 527 | 53 | A28G20C26T21 | A32G34C29T34 | A40G33C30T36 |
| A. baumannii | 51 | 529 | 53 | A28G20C26T21 | A32G34C29T34 | A40G33C30T36 |
| A. baumannii | 65 | 562 | 53 | A28G20C26T21 | A32G34C29T34 | A40G33C30T36 |
| A. baumannii | 68 | 579 | 53 | A28G20C26T21 | A32G34C29T34 | A40G33C30T36 |
| A. baumannii | 57 | 546 | 54 | A27G21C26T21 | A32G34C29T34 | A40G33C30T36 |
| A. baumannii | 58 | 548 | 54 | A27G21C26T21 | A32G34C29T34 | A40G33C30T36 |
| A. baumannii | 60 | 552 | 54 | A27G21C26T21 | A32G34C29T34 | A40G33C30T36 |
| A. baumannii | 61 | 555 | 54 | A27G21C26T21 | A32G34C29T34 | A40G33C30T36 |
| A. baumannii | 63 | 557 | 54 | A27G21C26T21 | A32G34C29T34 | A40G33C30T36 |
| A. baumannii | 66 | 570 | 54 | A27G21C26T21 | A32G34C29T34 | A40G33C30T36 |
| A. baumannii | 67 | 578 | 54 | A27G21C26T21 | A32G34C29T34 | A40G33C30T36 |
| A. baumannii | 69 | 584 | 54 | A27G21C26T21 | A32G34C29T34 | A40G33C30T36 |
| A. baumannii | 71 | 593 | 54 | A27G21C26T21 | A32G34C29T34 | A40G33C30T36 |
| A. baumannii | 72 | 602 | 54 | A27G21C26T21 | A32G34C29T34 | A40G33C30T36 |
| A. baumannii | 76 | 609 | 54 | A27G21C26T21 | A32G34C29T34 | A40G33C30T36 |
| A. baumannii | 78 | 621 | 54 | A27G21C26T21 | A32G34C29T34 | A40G33C30T36 |
| A. baumannii | 80 | 625 | 54 | A27G21C26T21 | A32G34C29T34 | A40G33C30T36 |
| A. baumannii | 81 | 628 | 54 | A27G21C26T21 | A32G34C29T34 | A40G33C30T36 |
| A. baumannii | 82 | 632 | 54 | A27G21C26T21 | A32G34C29T34 | A40G33C30T36 |
| A. baumannii | 84 | 649 | 54 | A27G21C26T21 | A32G34C29T34 | A40G33C30T36 |
| A. baumannii | 86 | 655 | 54 | A27G21C26T21 | A32G34C29T34 | A40G33C30T36 |
| A. baumannii | 88 | 668 | 54 | A27G21C26T21 | A32G34C29T34 | A40G33C30T36 |
| A. baumannii | 90 | 671 | 54 | A27G21C26T21 | A32G34C29T34 | A40G33C30T36 |
| A. baumannii | 91 | 672 | 54 | A27G21C26T21 | A32G34C29T34 | A40G33C30T36 |
| A. baumannii | 92 | 673 | 54 | A27G21C26T21 | A32G34C29T34 | A40G33C30T36 |
| A. baumannii | 18 | 196 | 55 | A27G21C25T22 | A31G36C27T35 | A40G33C29T37 |
| A. baumannii | 55 | 537 | 27 | A27G21C25T22 | A32G35C28T34 | A40G33C30T36 |
| A. baumannii | 28 | 263 | 27 | A27G21C25T22 | A32G35C28T34 | A40G33C30T36 |
| A. sp. 3 | 14 | 164 | B7 | A26G23C23T23 | A30G36C27T36 | A39G37C26T37 |
| mixture | 7 | 71 | ? | ND | ND | ND |

TABLE 18C
Base Compositions Determined from A. baumannii DNA Samples Obtained
from Northwestern Medical Center and Amplified with Triangulation
Genotyping Analysis Primer Pair Nos. 1167, 1170 and 1171

| Species | Ibis# | Isolate | ST | PP No: 1167 fumC | PP No: 1170 fumC | PP No: 1171 ppa |
|---|---|---|---|---|---|---|
| A. baumannii | 54 | 536 | 3 | A41G34C35T37 | A38G27C20T51 | A35G37C31T46 |
| A. baumannii | 87 | 665 | 3 | A41G34C35T37 | A38G27C20T51 | A35G37C31T46 |
| A. baumannii | 8 | 80 | 10 | A41G34C34T38 | A38G27C21T50 | A35G37C33T44 |
| A. baumannii | 9 | 91 | 10 | A41G34C34T38 | A38G27C21T50 | A35G37C33T44 |
| A. baumannii | 10 | 92 | 10 | A41G34C34T38 | A38G27C21T50 | A35G37C33T44 |
| A. baumannii | 11 | 131 | 10 | A41G34C34T38 | A38G27C21T50 | A35G37C33T44 |
| A. baumannii | 12 | 137 | 10 | A41G34C34T38 | A38G27C21T50 | A35G37C33T44 |
| A. baumannii | 21 | 218 | 10 | A41G34C34T38 | A38G27C21T50 | A35G37C33T44 |
| A. baumannii | 26 | 242 | 10 | A41G34C34T38 | A38G27C21T50 | A35G37C33T44 |
| A. baumannii | 94 | 678 | 10 | A41G34C34T38 | A38G27C21T50 | A35G37C33T44 |
| A. baumannii | 1 | 9 | 10 | A41G34C34T38 | A38G27C21T50 | A35G37C33T44 |
| A. baumannii | 2 | 13 | 10 | A41G34C34T38 | A38G27C21T50 | A35G37C33T44 |
| A. baumannii | 3 | 19 | 10 | A41G34C34T38 | A38G27C21T50 | A35G37C33T44 |
| A. baumannii | 4 | 24 | 10 | A41G34C34T38 | A38G27C21T50 | A35G37C33T44 |
| A. baumannii | 5 | 36 | 10 | A41G34C34T38 | A38G27C21T50 | A35G37C33T44 |
| A. baumannii | 6 | 39 | 10 | A41G34C34T38 | A38G27C21T50 | A35G37C33T44 |
| A. baumannii | 13 | 139 | 10 | A41G34C34T38 | A38G27C21T50 | A35G37C33T44 |
| A. baumannii | 15 | 165 | 10 | A41G34C34T38 | A38G27C21T50 | A35G37C33T44 |
| A. baumannii | 16 | 170 | 10 | A41G34C34T38 | A38G27C21T50 | A35G37C33T44 |
| A. baumannii | 17 | 186 | 10 | A41G34C34T38 | A38G27C21T50 | A35G37C33T44 |
| A. baumannii | 20 | 202 | 10 | A41G34C34T38 | A38G27C21T50 | A35G37C33T44 |
| A. baumannii | 22 | 221 | 10 | A41G34C34T38 | A38G27C21T50 | A35G37C33T44 |
| A. baumannii | 24 | 234 | 10 | A41G34C34T38 | A38G27C21T50 | A35G37C33T44 |
| A. baumannii | 25 | 239 | 10 | A41G34C34T38 | A38G27C21T50 | A35G37C33T44 |
| A. baumannii | 33 | 370 | 10 | A41G34C34T38 | A38G27C21T50 | A35G37C33T44 |
| A. baumannii | 34 | 389 | 10 | A41G34C34T38 | A38G27C21T50 | A35G37C33T44 |
| A. baumannii | 19 | 201 | 14 | A40G35C34T38 | A38G27C21T50 | A35G37C30T47 |
| A. baumannii | 27 | 257 | 51 | A40G35C34T38 | A38G27C21T50 | A35G37C30T47 |
| A. baumannii | 29 | 301 | 51 | A40G35C34T38 | A38G27C21T50 | A35G37C30T47 |
| A. baumannii | 31 | 354 | 51 | A40G35C34T38 | A38G27C21T50 | A35G37C30T47 |
| A. baumannii | 36 | 422 | 51 | A40G35C34T38 | A38G27C21T50 | A35G37C30T47 |
| A. baumannii | 37 | 424 | 51 | A40G35C34T38 | A38G27C21T50 | A35G37C30T47 |
| A. baumannii | 38 | 434 | 51 | A40G35C34T38 | A38G27C21T50 | A35G37C30T47 |
| A. baumannii | 39 | 473 | 51 | A40G35C34T38 | A38G27C21T50 | A35G37C30T47 |
| A. baumannii | 40 | 482 | 51 | A40G35C34T38 | A38G27C21T50 | A35G37C30T47 |
| A. baumannii | 44 | 512 | 51 | A40G35C34T38 | A38G27C21T50 | A35G37C30T47 |
| A. baumannii | 45 | 516 | 51 | A40G35C34T38 | A38G27C21T50 | A35G37C30T47 |
| A. baumannii | 47 | 522 | 51 | A40G35C34T38 | A38G27C21T50 | A35G37C30T47 |
| A. baumannii | 48 | 526 | 51 | A40G35C34T38 | A38G27C21T50 | A35G37C30T47 |
| A. baumannii | 50 | 528 | 51 | A40G35C34T38 | A38G27C21T50 | A35G37C30T47 |
| A. baumannii | 52 | 531 | 51 | A40G35C34T38 | A38G27C21T50 | A35G37C30T47 |
| A. baumannii | 53 | 533 | 51 | A40G35C34T38 | A38G27C21T50 | A35G37C30T47 |
| A. baumannii | 56 | 542 | 51 | A40G35C34T38 | A38G27C21T50 | A35G37C30T47 |
| A. baumannii | 59 | 550 | 51 | A40G35C34T38 | A38G27C21T50 | A35G37C30T47 |
| A. baumannii | 62 | 556 | 51 | A40G35C34T38 | A38G27C21T50 | A35G37C30T47 |
| A. baumannii | 64 | 557 | 51 | A40G35C34T38 | A38G27C21T50 | A35G37C30T47 |
| A. baumannii | 70 | 588 | 51 | A40G35C34T38 | A38G27C21T50 | A35G37C30T47 |
| A. baumannii | 73 | 603 | 51 | A40G35C34T38 | A38G27C21T50 | A35G37C30T47 |
| A. baumannii | 74 | 605 | 51 | A40G35C34T38 | A38G27C21T50 | A35G37C30T47 |
| A. baumannii | 75 | 606 | 51 | A40G35C34T38 | A38G27C21T50 | A35G37C30T47 |
| A. baumannii | 77 | 611 | 51 | A40G35C34T38 | A38G27C21T50 | A35G37C30T47 |
| A. baumannii | 79 | 622 | 51 | A40G35C34T38 | A38G27C21T50 | A35G37C30T47 |
| A. baumannii | 83 | 643 | 51 | A40G35C34T38 | A38G27C21T50 | A35G37C30T47 |
| A. baumannii | 85 | 653 | 51 | A40G35C34T38 | A38G27C21T50 | A35G37C30T47 |
| A. baumannii | 89 | 669 | 51 | A40G35C34T38 | A38G27C21T50 | A35G37C30T47 |
| A. baumannii | 93 | 674 | 51 | A40G35C34T38 | A38G27C21T50 | A35G37C30T47 |
| A. baumannii | 23 | 228 | 51 | A40G35C34T38 | A38G27C21T50 | A35G37C30T47 |
| A. baumannii | 32 | 369 | 52 | A40G35C34T38 | A38G27C21T50 | A35G37C31T46 |
| A. baumannii | 35 | 393 | 52 | A40G35C34T38 | A38G27C21T50 | A35G37C31T46 |
| A. baumannii | 30 | 339 | 53 | A40G35C35T37 | A38G27C21T50 | A35G37C31T46 |
| A. baumannii | 41 | 485 | 53 | A40G35C35T37 | A38G27C21T50 | A35G37C31T46 |
| A. baumannii | 42 | 493 | 53 | A40G35C35T37 | A38G27C21T50 | A35G37C31T46 |
| A. baumannii | 43 | 502 | 53 | A40G35C35T37 | A38G27C21T50 | A35G37C31T46 |
| A. baumannii | 46 | 520 | 53 | A40G35C35T37 | A38G27C21T50 | A35G37C31T46 |
| A. baumannii | 49 | 527 | 53 | A40G35C35T37 | A38G27C21T50 | A35G37C31T46 |
| A. baumannii | 51 | 529 | 53 | A40G35C35T37 | A38G27C21T50 | A35G37C31T46 |
| A. baumannii | 65 | 562 | 53 | A40G35C35T37 | A38G27C21T50 | A35G37C31T46 |
| A. baumannii | 68 | 579 | 53 | A40G35C35T37 | A38G27C21T50 | A35G37C31T46 |
| A. baumannii | 57 | 546 | 54 | A40G35C34T38 | A39G26C22T49 | A35G37C31T46 |
| A. baumannii | 58 | 548 | 54 | A40G35C34T38 | A39G26C22T49 | A35G37C31T46 |
| A. baumannii | 60 | 552 | 54 | A40G35C34T38 | A39G26C22T49 | A35G37C31T46 |
| A. baumannii | 61 | 555 | 54 | A40G35C34T38 | A39G26C22T49 | A35G37C31T46 |
| A. baumannii | 63 | 557 | 54 | A40G35C34T38 | A39G26C22T49 | A35G37C31T46 |
| A. baumannii | 66 | 570 | 54 | A40G35C34T38 | A39G26C22T49 | A35G37C31T46 |
| A. baumannii | 67 | 578 | 54 | A40G35C34T38 | A39G26C22T49 | A35G37C31T46 |
| A. baumannii | 69 | 584 | 54 | A40G35C34T38 | A39G26C22T49 | A35G37C31T46 |
| A. baumannii | 71 | 593 | 54 | A40G35C34T38 | A39G26C22T49 | A35G37C31T46 |
| A. baumannii | 72 | 602 | 54 | A40G35C34T38 | A39G26C22T49 | A35G37C31T46 |
| A. baumannii | 76 | 609 | 54 | A40G35C34T38 | A39G26C22T49 | A35G37C31T46 |
| A. baumannii | 78 | 621 | 54 | A40G35C34T38 | A39G26C22T49 | A35G37C31T46 |
| A. baumannii | 80 | 625 | 54 | A40G35C34T38 | A39G26C22T49 | A35G37C31T46 |
| A. baumannii | 81 | 628 | 54 | A40G35C34T38 | A39G26C22T49 | A35G37C31T46 |
| A. baumannii | 82 | 632 | 54 | A40G35C34T38 | A39G26C22T49 | A35G37C31T46 |
| A. baumannii | 84 | 649 | 54 | A40G35C34T38 | A39G26C22T49 | A35G37C31T46 |
| A. baumannii | 86 | 655 | 54 | A40G35C34T38 | A39G26C22T49 | A35G37C31T46 |
| A. baumannii | 88 | 668 | 54 | A40G35C34T38 | A39G26C22T49 | A35G37C31T46 |
| A. baumannii | 90 | 671 | 54 | A40G35C34T38 | A39G26C22T49 | A35G37C31T46 |
| A. baumannii | 91 | 672 | 54 | A40G35C34T38 | A39G26C22T49 | A35G37C31T46 |
| A. baumannii | 92 | 673 | 54 | A40G35C34T38 | A39G26C22T49 | A35G37C31T46 |
| A. baumannii | 18 | 196 | 55 | A42G34C33T38 | A38G27C20T51 | A35G37C31T46 |
| A. baumannii | 55 | 537 | 27 | A40G35C33T39 | A38G27C20T51 | A35G37C33T44 |
| A. baumannii | 28 | 263 | 27 | A40G35C33T39 | A38G27C20T51 | A35G37C33T44 |
| A. sp. 3 | 14 | 164 | B7 | A43G37C30T37 | A36G27C24T49 | A34G37C31T47 |
| mixture | 7 | 71 | — | ND | ND | ND |

Base composition analysis of the samples obtained from Walter Reed hospital indicated that a majority of the strain types identified were the same strain types already characterized by the OIF study of Example 12. This is not surprising since at least some patients from which clinical samples were obtained in OIF were transferred to the Walter Reed Hospital (WRAIR). Examples of these common strain types include: ST10, ST11, ST12, ST14, ST15, ST16 and ST46. A strong correlation was noted between these strain types and the presence of mutations in the gyrA and parC which confer quinolone drug resistance.

In contrast, the results of base composition analysis of samples obtained from Northwestern Medical Center indicate the presence of 4 major strain types: ST10, ST51, ST53 and ST54. All of these strain types have the gyrA quinolone resistance mutation and most also have the parC quinolone resistance mutation, with the exception of ST35. This observation is consistent with the current understanding that the gyrA mutation generally appears before the parC mutation and suggests that the acquisition of these drug resistance mutations is rather recent and that resistant isolates are taking over the wild-type isolates. Another interesting observation was that a single isolate of ST3 (isolate 841) displays a triangulation genotyping analysis pattern similar to other isolates of ST3, but the codon analysis amplification product base compositions indicate that this isolate has not yet undergone the quinolone resistance mutations in gyrA and parC.

The six isolates that represent species other than Acinetobacter baumannii in the samples obtained from the Walter Reed Hospital were each found to not carry the drug resistance mutations.

The results described above involved analysis of 183 samples using the methods and compositions of the present invention. Results were provided to collaborators at the Walter Reed hospital and Northwestern Medical center within a week of obtaining samples. This example highlights the rapid throughput characteristics of the analysis platform and the resolving power of triangulation genotyping analysis and codon analysis for identification of and determination of drug resistance in bacteria.

### Example 14

### Identification of Drug Resistance Genes and Virulence Factors in Staphylococcus aureus

Three primer pair panels, each comprising eight primer pairs, were configured for identification of the Staphylococcus aureus species and for identification of drug resistance genes and virulence factors of Staphylococcus aureus bioagents. These panels are shown in Tables 19A, 19B and 19C. The primer sequences in these panels can also be found in Table 2, and are cross-referenced in Tables 19A-C by primer pair numbers, primer pair names, and SEQ ID NOs.

TABLE 19A
Panel of Primer Pairs for Identification of Drug Resistance Genes and Virulence
Factors in Staphylococcus aureus

| | | Forward | | Reverse | |
|---|---|---|---|---|---|
| Primer | | Primer | | Primer | |
| Pair | | (SEQ ID | | (SEQ ID | Target |
| No. | Forward Primer Name | NO:) | Reverse Primer Name | NO:) | Gene |
| 879 | MECA_Y14051_4507_4530_F | 288 | MECA_Y14051_4555_4581_R | 1269 | mecA |
| 2056 | MECI-R_NC003923-41798- | 698 | MECI-R_NC003923-41798- | 1420 | MecI-R |
| | 41609_33_60_F | | 41609_86_113_R | | |
| 2081 | ERMA_NC002952-55890- | 217 | ERMA_NC002952-55890- | 1167 | ermA |
| | 56621_366_395_F | | 56621_438_465_R | | |
| 2086 | ERMC_NC005908-2004- | 399 | ERMC_NC005908-2004- | 1041 | ermC |
| | 2738_85_116_F | | 2738_173_206_R | | |
| 2095 | PVLUK_NC003923-1529595- | 456 | PVLUK_NC003923-1529595- | 1261 | Pv-luk |
| | 1531285_688_713_F | | 1531285_775_804_R | | |
| 2249 | TUFB_NC002758-615038- | 430 | TUFB_NC002758-615038- | 1321 | tufB |
| | 616222_696_725_F | | 616222_793_820_R | | |
| 2256 | NUC_NC002758-894288- | 174 | NUC_NC002758-894288- | 853 | Nuc |
| | 894974_316-345_F | | 894974_396_421_R | | |
| 2313 | MUPR_X75439_2486_2516_F | 172 | MUPR_X75439_2548_2574_R | 1360 | mupR |

TABLE 19B
Panel of Primer Pairs for Identification of Drug Resistance Genes and Virulence
Factors in Staphylococcus aureus

| | | Forward | | Reverse | |
|---|---|---|---|---|---|
| Primer | | Primer | | Primer | |
| Pair | | (SEQ ID | | (SEQ ID | Target |
| No. | Forward Primer Name | NO:) | Reverse Primer Name | NO:) | Gene |
| 879 | MECA_Y14051_4507_4530_F | 288 | MECA_Y14051_4555_4581_R | 1269 | mecA |
| 2056 | MECI-R_NC003923-41798- | 698 | MECI-R_NC003923-41798- | 1420 | MecI-R |
| | 41609_33_60_F | | 41609_86_113_R | | |
| 2081 | ERMA_NC002952-55890- | 217 | ERMA_NC002952-55890- | 1167 | ermA |
| | 56621_366_395_F | | 56621_438_465_R | | |
| 2086 | ERMC_NC005908-2004- | 399 | ERMC_NC005908-2004- | 1041 | ermC |
| | 2738_85_116_F | | 2738_173_206_R | | |
| 2095 | PVLUK_NC003923-1529595- | 456 | PVLUK_NC003923-1529595- | 1261 | Pv-luk |
| | 1531285_688_713_F | | 1531285_775_804_R | | |
| 2249 | TUFB_NC002758-615038- | 430 | TUFB_NC002758-615038- | 1321 | tufB |
| | 616222_696_725_F | | 616222_793_820_R | | |
| 2256 | NUC_NC002758-894288- | 174 | NUC_NC002758-894288- | 853 | Nuc |
| | 894974_316_345_F | | 894974_396_421_R | | |
| 3016 | MUPR_X75439_2482_2510_F | 205 | MUPR_X75439_2551_2573_R | 876 | mupR |

TABLE 19C
Panel of Primer Pairs for Identification of Drug Resistance Genes and Virulence
Factors in Staphylococcus aureus

| | | Forward | | Reverse | |
|---|---|---|---|---|---|
| Primer | | Primer | | Primer | |
| Pair | | (SEQ ID | | (SEQ ID | Target |
| No. | Forward Primer Name | NO:) | Reverse Primer Name | NO:) | Gene |
| 879 | MECA_Y14051_4507_4530_F | 288 | MECA_Y14051_4555_4581_R | 1269 | mecA |
| 2056 | MECI-R_NC003923-41798- | 698 | MECI-R_NC003923-41798- | 1420 | MecI-R |
| | 41609_33_60_F | | 41609_86_113_R | | |
| 2081 | ERMA_NC002952-55890- | 217 | ERMA_NC002952-55890- | 1167 | ermA |
| | 56621_366_395_F | | 56621_438_465_R | | |
| 2086 | ERMC_NC005908-2004- | 399 | ERMC_NC005908-2004- | 1041 | ermC |
| | 2738_85_116_F | | 2738_173_206_R | | |
| 2095 | PVLUK_NC003923-1529595- | 456 | PVLUK_NC003923-1529595- | 1261 | Pv-luk |
| | 1531285_688_713_F | | 1531285_775_804_R | | |
| 2249 | TUFB_NC002758-615038- | 430 | TUFB_NC002758-615038- | 1321 | tufB |
| | 616222_696_725_F | | 616222_793_820_R | | |
| 2256 | NUC_NC002758-894288- | 174 | NUC_NC002758-894288- | 853 | Nuc |
| | 894974_316_345_F | | 894974_396_421_R | | |
| 3106 | TSST1_NC002758.2- | 1465 | TSST1_NC002758.2- | 1466 | tsst1 |
| | 2137509-2138213_519_546_F | | 2137509-2138213_593-620_R | | |

Primer pair numbers 2256 and 2249 are confirmation primers configured with the aim of high-level identification of Staphylococcus aureus. The nuc gene is a Staphylococcus aureus-specific marker gene. The tufB gene is a universal housekeeping gene but the bioagent identifying amplicon defined by primer pair number 2249 provides a unique base composition (A43 G28 C19 T35) which distinguishes Staphylococcus aureus from other members of the genus Staphylococcus.

High level methicillin resistance in a given strain of Staphylococcus aureus is indicated by bioagent identifying amplicons defined by primer pair numbers 879 and 2056. Analyses have indicated that primer pair number 879 is not expected to prime S. sciuri homolog or Enterococcus faecalis/faciem ampicillin-resistant PBP5 homologs.

Macrolide and erythromycin resistance in a given strain of Staphylococcus aureus is indicated by bioagent identifying amplicons defined by primer pair numbers 2081 and 2086.

Resistance to mupriocin in a given strain of Staphylococcus aureus is indicated by bioagent identifying amplicons defined by primer pair numbers 2313 and 3016.

In the above panels, virulence in a given strain of Staphylococcus aureus can be indicated by bioagent identifying amplicons defined by primer pair numbers 2095 and 3106. Primer pair number 2095 can identify both the pvl (lukS-PV) gene and the lukD gene which encodes a homologous enterotoxin. A bioagent identifying amplicon of the lukD gene defined by primer pair number 2095 has a six nucleobase length difference relative to the lukS-PV gene. Further, primer pair number 3106 is configured to generate amplicons within the tsst-1 gene, which encodes for shock syndrome toxin, which causes toxic shock syndrome (TSS).

A total of 32 blinded samples of different strains of Staphylococcus aureus were provided by the Center for Disease Control (CDC). Each sample was analyzed by PCR amplification with the eight primer pair panel of Table 19A, followed by purification and measurement of molecular masses of the amplification products by mass spectrometry. Base compositions for the amplification products were calculated. The base compositions provide the information summarized above for each primer pair. The results are shown in Tables 20A and 20B. One result noted upon un-blinding of the samples is that each of the PVL+ identifications agreed with PVL+ identified in the same samples by standard PCR assays. These results indicate that the panel of eight primer pairs is useful for identification of drug resistance and virulence sub-species characteristics for Staphylococcus aureus. Thus, it is expected that a kit comprising one or more of the members of the panels provided in Tables 19A-C will be a useful embodiment.

TABLE 20A
Drug Resistance and Virulence Identified in Blinded Samples of
Various Strains of Staphylococcus aureus with Primer Pair Nos.
2081, 2086, 2095 and 2256

| | Primer | Primer | Primer | Primer |
|---|---|---|---|---|
| | Pair | Pair | Pair | Pair |
| Sample | No. 2081 | No. 2086 | No. 2095 | No. 2256 |
| Index No. | (ermA) | (ermC) | (pv-luk) | (nuc) |
| CDC0010 | − | − | PVL−/lukD+ | + |
| CDC0015 | − | − | PVL+/lukD+ | + |
| CDC0019 | − | + | PVL−/lukD+ | + |
| CDC0026 | + | − | PVL−/lukD+ | + |
| CDC0030 | + | − | PVL−/lukD+ | + |
| CDC004 | − | − | PVL+/lukD+ | + |
| CDC0014 | − | + | PVL+/lukD+ | + |
| CDC008 | − | − | PVL−/lukD+ | + |
| CDC001 | + | − | PVL−/lukD+ | + |
| CDC0022 | + | − | PVL−/lukD+ | + |
| CDC006 | + | − | PVL−/lukD+ | + |
| CDC007 | − | − | PVL−/lukD+ | + |
| CDCVRSA1 | + | − | PVL−/lukD+ | + |
| CDCVRSA2 | + | + | PVL−/lukD+ | + |
| CDC0011 | + | − | PVL−/lukD+ | + |
| CDC0012 | − | − | PVL+/lukD− | + |
| CDC0021 | + | − | PVL−/lukD+ | + |
| CDC0023 | + | − | PVL−/lukD+ | + |
| CDC0025 | + | − | PVL−/lukD+ | + |
| CDC005 | − | − | PVL−/lukD+ | + |
| CDC0018 | + | − | PVL+/lukD− | + |
| CDC002 | − | − | PVL−/lukD+ | + |
| CDC0028 | + | − | PVL−/lukD+ | + |
| CDC003 | − | − | PVL−/lukD+ | + |
| CDC0013 | − | − | PVL+/lukD+ | + |
| CDC0016 | − | − | PVL−/lukD+ | + |
| CDC0027 | + | − | PVL−/lukD+ | + |
| CDC0029 | − | − | PVL+/lukD+ | + |
| CDC0020 | − | + | PVL−/lukD+ | + |
| CDC0024 | − | − | PVL−/lukD+ | + |
| CDC0031 | − | − | PVL−/lukD+ | + |

TABLE 20B
Drug Resistance and Virulence Identified in Blinded Samples of Various
Strains of Staphylococcus aureus with Primer Pair Nos. 2249, 879, 2056,
and 2313

| | | Primer Pair | Primer Pair | Primer Pair |
|---|---|---|---|---|
| Sample | Primer Pair No. 2249 | No. 879 | No. 2056 | No. 2313 |
| Index No. | (tufB) | (mecA) | (mecI-R) | (mupR) |
| CDC0010 | Staphylococcus aureus | + | + | − |
| CDC0015 | Staphylococcus aureus | − | − | − |
| CDC0019 | Staphylococcus aureus | + | + | − |
| CDC0026 | Staphylococcus aureus | + | + | − |
| CDC0030 | Staphylococcus aureus | + | + | − |
| CDC004 | Staphylococcus aureus | + | + | − |
| CDC0014 | Staphylococcus aureus | + | + | − |
| CDC008 | Staphylococcus aureus | + | + | − |
| CDC001 | Staphylococcus aureus | + | + | − |
| CDC0022 | Staphylococcus aureus | + | + | − |
| CDC006 | Staphylococcus aureus | + | + | + |
| CDC007 | Staphylococcus aureus | + | + | − |
| CDCVRSA1 | Staphylococcus aureus | + | + | − |
| CDCVRSA2 | Staphylococcus aureus | + | + | − |
| CDC0011 | Staphylococcus aureus | − | − | − |
| CDC0012 | Staphylococcus aureus | + | + | − |
| CDC0021 | Staphylococcus aureus | + | + | − |
| CDC0023 | Staphylococcus aureus | + | + | − |
| CDC0025 | Staphylococcus aureus | + | + | − |
| CDC005 | Staphylococcus aureus | + | + | − |
| CDC0018 | Staphylococcus aureus | + | + | − |
| CDC002 | Staphylococcus aureus | + | + | − |
| CDC0028 | Staphylococcus aureus | + | + | − |
| CDC003 | Staphylococcus aureus | + | + | − |
| CDC0013 | Staphylococcus aureus | + | + | − |
| CDC0016 | Staphylococcus aureus | + | + | − |
| CDC0027 | Staphylococcus aureus | + | + | − |
| CDC0029 | Staphylococcus aureus | + | + | − |
| CDC0020 | Staphylococcus aureus | − | − | − |
| CDC0024 | Staphylococcus aureus | + | + | − |
| CDC0031 | Staphylococcus scleiferi | − | − | − |

### Example 15

### Selection and Use of Triangulation Genotyping Analysis Primer Pairs for Staphylococcus aureus

To combine the power of high-throughput mass spectrometric analysis of bioagent identifying amplicons with the sub-species characteristic resolving power provided by triangulation genotyping analysis, two panels, each with eight triangulation genotyping analysis primer pairs was selected and are listed in Tables 21A and 21B. The primer pairs are configured to produce bioagent identifying amplicons within six different housekeeping genes which are listed in the tables. The primer sequences are found in Table 2 and are cross-referenced by the primer pair numbers, primer pair names or SEQ ID NOs listed in Tables 21A and 21B. Further, another panel of primer pairs was developed to combining the identification/drug resistance/viulence identifying power of the primer pairs of Tables 19A-C with the triangulation genotyping analysis of Tables 21A-B. This panel comprises sixteen primer pairs and is shown in Table 21C. The panel shown in Table 21C combines primer pairs of Tables 19B and 21B. However, other combinations of primer pairs from the Staphylococcus aureus genotyping panels and the identification/virulence/drug resistant panels shown in Examples 14 and 15 are encompassed by this disclosure.

TABLE 21A
Primer Pairs for Triangulation Genotyping Analysis of Staphylococcus aureus

| | | Forward | | Reverse | |
|---|---|---|---|---|---|
| Primer | | Primer | | Primer | |
| Pair | | (SEQ ID | | (SEQ ID | Target |
| No. | Forward Primer Name | NO:) | Reverse Primer Name | NO:) | Gene |
| 2146 | ARCC_NC003923-2725050- | 437 | ARCC_NC003923-2725050- | 1137 | arcC |
| | 2724595_131_161_F | | 2724595_214_245_R | | |
| 2149 | AROE_NC003923-1674726- | 530 | AROE_NC003923-1674726- | 891 | aroE |
| | 1674277_30_62_F | | 1674277_155_181_R | | |
| 2150 | AROE_NC003923-1674726- | 474 | AROE_NC003923-1674726- | 869 | aroE |
| | 1674277_204_232_F | | 1674277_308_335_R | | |
| 2156 | GMK_NC003923-1190906- | 268 | GMK_NC003923-1190906- | 1284 | gmk |
| | 1191334_301_329_F | | 1191334_403_432_R | | |
| 2157 | PTA_NC003923-628885- | 418 | PTA_NC003923-628885- | 1301 | pta |
| | 629355_237_263_F | | 629355_314_345_R | | |
| 2161 | TPI_NC003923-830671- | 318 | TPI_NC003923-830671- | 1300 | tpi |
| | 831072_1_34_F | | 831072_97_129_R | | |
| 2163 | YQI_NC003923-378916- | 440 | YQI_NC003923-378916- | 1076 | yqi |
| | 379431_142_167_F | | 379431_259_284_R | | |
| 2166 | YQI_NC003923-378916- | 219 | YQI_NC003923-378916- | 1013 | yqi |
| | 379431_275_300_F | | 379431_364_396_R | | |

TABLE 21B
Primer Pairs for Triangulation Genotyping Analysis of Staphylococcus aureus

| | | Forward | | Reverse | |
|---|---|---|---|---|---|
| Primer | | Primer | | Primer | |
| Pair | | (SEQ ID | | (SEQ ID | Target |
| No. | Forward Primer Name | NO:) | Reverse Primer Name | NO:) | Gene |
| 3025 | ARCC_NC003923-2725050- | 437 | ARCC_NC003923-2725050- | 1232 | arcC |
| | 2724595_131_161_F | | 2724595_232_260_R | | |
| 2149 | AROE_NC003923-1674726- | 530 | AROE_NC003923-1674726- | 891 | aroE |
| | 1674277_30_62_F | | 1674277_155_181_R | | |
| 2150 | AROE_NC003923-1674726- | 474 | AROE_NC003923-1674726- | 869 | aroE |
| | 1674277_204_232_F | | 1674277_308_335_R | | |
| 2156 | GMK_NC003923-1190906- | 268 | GMK_NC003923-1190906- | 1284 | gmk |
| | 1191334_301_329_F | | 1191334_403_432_R | | |
| 2157 | PTA_NC003923-628885- | 418 | PTA_NC003923-628885- | 1301 | pta |
| | 629355_237_263_F | | 629355_314_345_R | | |
| 2161 | TPI_NC003923-830671- | 318 | TPI_NC003923-830671- | 1300 | tpi |
| | 831072_1_34_F | | 831072_97_129_R | | |
| 2163 | YQI_NC003923-378916- | 440 | YQI_NC003923-378916- | 1076 | yqi |
| | 379431_142_167_F | | 379431_259_284_R | | |
| 2166 | YQI_NC003923-378916- | 219 | YQI_NC003923-378916- | 1013 | yqi |
| | 379431_275_300_F | | 379431_364_396_R | | |

TABLE 21C
Panel of Primer Pairs for Identification/Drug Resistance/Virulence and Triangulation Genotyping
Analysis of Staphylococcus aureus

| | | Forward | | Reverse | |
|---|---|---|---|---|---|
| Primer | | Primer | | Primer | |
| Pair | | (SEQ ID | | (SEQ ID | Target |
| No. | Forward Primer Name | NO:) | Reverse Primer Name | NO:) | Gene |
| 3025 | ARCC_NC003923-2725050- | 437 | ARCC_NC003923-2725050- | 1232 | arcC |
| | 2724595_131_161_F | | 2724595_232_260_R | | |
| 2149 | AROE_NC003923-1674726- | 530 | AROE_NC003923-1674726- | 891 | aroE |
| | 1674277_30_62_F | | 1674277_155_181_R | | |
| 2150 | AROE_NC003923-1674726- | 474 | AROE_NC003923-1674726- | 869 | aroE |
| | 1674277_204_232_F | | 1674277_308_335_R | | |
| 2156 | GMK_NC003923-1190906- | 268 | GMK_NC003923-1190906- | 1284 | gmk |
| | 1191334_301_329_F | | 1191334_403_432_R | | |
| 2157 | PTA_NC003923-628885- | 418 | PTA_NC003923-628885- | 1301 | pta |
| | 629355_237_263_F | | 629355_314_345_R | | |
| 2161 | TPI_NC003923-830671- | 318 | TPI_NC003923-830671- | 1300 | tpi |
| | 831072_1_34_F | | 831072_97_129_R | | |
| 2163 | YQI_NC003923-378916- | 440 | YQI_NC003923-378916- | 1076 | yqi |
| | 379431_142_167_F | | 379431_259_284_R | | |
| 2166 | YQI_NC003923-378916- | 219 | YQI_NC003923-378916- | 1013 | yqi |
| | 379431_275_300_F | | 379431_364_396_R | | |
| 879 | MECA_Y14051_4507_4530_F | 288 | MECA_Y14051_4555_4581_R | 1269 | mecA |
| 2056 | MECI-R_NC003923-41798- | 698 | MECI-R_NC003923-41798- | 1420 | MecI-R |
| | 41609_33_60_F | | 41609_86_113_R | | |
| 2081 | ERMA_NC002952-55890- | 217 | ERMA_NC002952-55890- | 1167 | ermA |
| | 56621_366_395_F | | 56621_438_465_R | | |
| 2086 | ERMC_NC005908-2004- | 399 | ERMC_NC005908-2004- | 1041 | ermC |
| | 2738_85_116_F | | 2738_173_206_R | | |
| 2095 | PVLUK_NC003923-1529595- | 456 | PVLUK_NC003923-1529595- | 1261 | Pv-luk |
| | 1531285_688_713_F | | 1531285_775_804_R | | |
| 2249 | TUFB_NC002758-615038- | 430 | TUFB_NC002758-615038- | 1321 | tufB |
| | 616222_696_725_F | | 616222_793_820_R | | |
| 2256 | NUC_NC002758-894288- | 174 | NUC_NC002758-894288- | 853 | Nuc |
| | 894974_316_345_F | | 894974_396_421_R | | |
| 3016 | MUPR_X75439_2482_2510_F | 205 | MUPR_X75439_2551_2573_R | 876 | mupR |

The same samples analyzed for drug resistance and virulence in Example 14 were subjected to triangulation genotyping analysis. The primer pairs of Table 21A were used to produce amplification products by PCR, which were subsequently purified and measured by mass spectrometry. Base compositions were calculated from the molecular masses and are shown in Tables 22A and 22B.

TABLE 22A
Triangulation Genotyping Analysis of Blinded Samples of Various Strains of Staphylococcus aureus
with Primer Pair Nos. 2146, 2149, 2150 and 2156

| Sample | | Primer Pair | Primer Pair | Primer Pair | Primer Pair |
|---|---|---|---|---|---|
| Index | | No. 2146 | No. 2149 | No. 2150 | No. 2156 |
| No. | Strain | (arcC) | (aroE) | (aroE) | (gmk) |
| CDC0010 | COL | A44 G24 C18 T29 | A59 G24 C18 T51 | A40 G36 C13 T43 | A50 G30 C20 T32 |
| CDC0015 | COL | A44 G24 C18 T29 | A59 G24 C18 T51 | A40 G36 C13 T43 | A50 G30 C20 T32 |
| CDC0019 | COL | A44 G24 C18 T29 | A59 G24 C18 T51 | A40 G36 C13 T43 | A50 G30 C20 T32 |
| CDC0026 | COL | A44 G24 C18 T29 | A59 G24 C18 T51 | A40 G36 C13 T43 | A50 G30 C20 T32 |
| CDC0030 | COL | A44 G24 C18 T29 | A59 G24 C18 T51 | A40 G36 C13 T43 | A50 G30 C20 T32 |
| CDC004 | COL | A44 G24 C18 T29 | A59 G24 C18 T51 | A40 G36 C13 T43 | A50 G30 C20 T32 |
| CDC0014 | COL | A44 G24 C18 T29 | A59 G24 C18 T51 | A40 G36 C13 T43 | A50 G30 C20 T32 |
| CDC008 | ???? | A44 G24 C18 T29 | A59 G24 C18 T51 | A40 G36 C13 T43 | A50 G30 C20 T32 |
| CDC001 | Mu50 | A45 G23 C20 T27 | A58 G24 C18 T52 | A40 G36 C13 T43 | A51 G29 C21 T31 |
| CDC0022 | Mu50 | A45 G23 C20 T27 | A58 G24 C18 T52 | A40 G36 C13 T43 | A51 G29 C21 T31 |
| CDC006 | Mu50 | A45 G23 C20 T27 | A58 G24 C18 T52 | A40 G36 C13 T43 | A51 G29 C21 T31 |
| CDC0011 | MRSA252 | A45 G24 C18 T28 | A58 G24 C19 T51 | A41 G36 C12 T43 | A51 G29 C21 T31 |
| CDC0012 | MRSA252 | A45 G24 C18 T28 | A58 G24 C19 T51 | A41 G36 C12 T43 | A51 G29 C21 T31 |
| CDC0021 | MRSA252 | A45 G24 C18 T28 | A58 G24 C19 T51 | A41 G36 C12 T43 | A51 G29 C21 T31 |
| CDC0023 | ST: 110 | A45 G24 C18 T28 | A59 G24 C18 T51 | A40 G36 C13 T43 | A50 G30 C20 T32 |
| CDC0025 | ST: 110 | A45 G24 C18 T28 | A59 G24 C18 T51 | A40 G36 C13 T43 | A50 G30 C20 T32 |
| CDC005 | ST: 338 | A44 G24 C18 T29 | A59 G23 C19 T51 | A40 G36 C14 T42 | A51 G29 C21 T31 |
| CDC0018 | ST: 338 | A44 G24 C18 T29 | A59 G23 C19 T51 | A40 G36 C14 T42 | A51 G29 C21 T31 |
| CDC002 | ST: 108 | A46 G23 C20 T26 | A58 G24 C19 T51 | A42 G36 C12 T42 | A51 G29 C20 T32 |
| CDC0028 | ST: 108 | A46 G23 C20 T26 | A58 G24 C19 T51 | A42 G36 C12 T42 | A51 G29 C20 T32 |
| CDC003 | ST: 107 | A45 G23 C20 T27 | A58 G24 C18 T52 | A40 G36 C13 T43 | A51 G29 C21 T31 |
| CDC0013 | ST: 12 | ND | A59 G24 C18 T51 | A40 G36 C13 T43 | A51 G29 C21 T31 |
| CDC0016 | ST: 120 | A45 G23 C18 T29 | A58 G24 C19 T51 | A40 G37 C13 T42 | A51 G29 C21 T31 |
| CDC0027 | ST: 105 | A45 G23 C20 T27 | A58 G24 C18 T52 | A42 G36 C13 T43 | A51 G29 C21 T31 |
| CDC0029 | MSSA476 | A45 G23 C20 T27 | A58 G24 C19 T51 | A40 G36 C13 T43 | A50 G30 C20 T32 |
| CDC0020 | ST: 15 | A44 G23 C21 T27 | A59 G23 C18 T52 | A40 G36 C13 T43 | A50 G30 C20 T32 |
| CDC0024 | ST: 137 | A45 G23 C20 T27 | A57 G25 C19 T51 | A40 G36 C13 T43 | A51 G29 C22 T30 |
| CDC0031 | *** | No product | No product | No product | No product |

TABLE 22B
Triangulation Genotyping Analysis of Blinded Samples of Various Strains of Staphylococcus aureus
with Primer Pair Nos. 2146, 2149, 2150 and 2156

| Sample | | Primer Pair | Primer Pair | Primer Pair | Primer Pair |
|---|---|---|---|---|---|
| Index | | No. 2157 | No. 2161 | No. 2163 | No. 2166 |
| No. | Strain | (pta) | (tpi) | (yqi) | (yqi) |
| CDC0010 | COL | A32 G25 C23 T29 | A51 G28 C22 T28 | A41 G37 C22 T43 | A37 G30 C18 T37 |
| CDC0015 | COL | A32 G25 C23 T29 | A51 G28 C22 T28 | A41 G37 C22 T43 | A37 G30 C18 T37 |
| CDC0019 | COL | A32 G25 C23 T29 | A51 G28 C22 T28 | A41 G37 C22 T43 | A37 G30 C18 T37 |
| CDC0026 | COL | A32 G25 C23 T29 | A51 G28 C22 T28 | A41 G37 C22 T43 | A37 G30 C18 T37 |
| CDC0030 | COL | A32 G25 C23 T29 | A51 G28 C22 T28 | A41 G37 C22 T43 | A37 G30 C18 T37 |
| CDC004 | COL | A32 G25 C23 T29 | A51 G28 C22 T28 | A41 G37 C22 T43 | A37 G30 C18 T37 |
| CDC0014 | COL | A32 G25 C23 T29 | A51 G28 C22 T28 | A41 G37 C22 T43 | A37 G30 C18 T37 |
| CDC008 | unknown | A32 G25 C23 T29 | A51 G28 C22 T28 | A41 G37 C22 T43 | A37 G30 C18 T37 |
| CDC001 | Mu50 | A33 G25 C22 T29 | A50 G28 C22 T29 | A42 G36 C22 T43 | A36 G31 C19 T36 |
| CDC0022 | Mu50 | A33 G25 C22 T29 | A50 G28 C22 T29 | A42 G36 C22 T43 | A36 G31 C19 T36 |
| CDC006 | Mu50 | A33 G25 C22 T29 | A50 G28 C22 T29 | A42 G36 C22 T43 | A36 G31 C19 T36 |
| CDC0011 | MRSA252 | A32 G25 C23 T29 | A50 G28 C22 T29 | A42 G36 C22 T43 | A37 G30 C18 T37 |
| CDC0012 | MRSA252 | A32 G25 C23 T29 | A50 G28 C22 T29 | A42 G36 C22 T43 | A37 G30 C18 T37 |
| CDC0021 | MRSA252 | A32 G25 C23 T29 | A50 G28 C22 T29 | A42 G36 C22 T43 | A37 G30 C18 T37 |
| CDC0023 | ST: 110 | A32 G25 C23 T29 | A51 G28 C22 T28 | A41 G37 C22 T43 | A37 G30 C18 T37 |
| CDC0025 | ST: 110 | A32 G25 C23 T29 | A51 G28 C22 T28 | A41 G37 C22 T43 | A37 G30 C18 T37 |
| CDC005 | ST: 338 | A32 G25 C24 T28 | A51 G27 C21 T30 | A42 G36 C22 T43 | A37 G30 C18 T37 |
| CDC0018 | ST: 338 | A32 G25 C24 T28 | A51 G27 C21 T30 | A42 G36 C22 T43 | A37 G30 C18 T37 |
| CDC002 | ST: 108 | A33 G25 C23 T28 | A50 G28 C22 T29 | A42 G36 C22 T43 | A37 G30 C18 T37 |
| CDC0028 | ST: 108 | A33 G25 C23 T28 | A50 G28 C22 T29 | A42 G36 C22 T43 | A37 G30 C18 T37 |
| CDC003 | ST: 107 | A32 G25 C23 T29 | A51 G28 C22 T28 | A41 G37 C22 T43 | A37 G30 C18 T37 |
| CDC0013 | ST: 12 | A32 G25 C23 T29 | A51 G28 C22 T28 | A42 G36 C22 T43 | A37 G30 C18 T37 |
| CDC0016 | ST: 120 | A32 G25 C24 T28 | A50 G28 C21 T30 | A42 G36 C22 T43 | A37 G30 C18 T37 |
| CDC0027 | ST: 105 | A33 G25 C22 T29 | A50 G28 C22 T29 | A43 G36 C21 T43 | A36 G31 C19 T36 |
| CDC0029 | MSSA476 | A33 G25 C22 T29 | A50 G28 C22 T29 | A42 G36 C22 T43 | A36 G31 C19 T36 |
| CDC0020 | ST: 15 | A33 G25 C22 T29 | A50 G28 C21 T30 | A42 G36 C22 T43 | A36 G31 C18 T37 |
| CDC0024 | ST: 137 | A33 G25 C22 T29 | A51 G28 C22 T28 | A42 G36 C22 T43 | A37 G30 C18 T37 |
| CDC0031 | *** | A34 G25 C25 T25 | A51 G27 C24 T27 | No product | No product |

Note:
*** The sample CDC0031 was identified as Staphylococcus scleiferi as indicated in Example 14. Thus, the triangulation genotyping primers configured for Staphylococcus aureus would generally not be expected to prime and produce amplification products of this organism. Tables 22A and 22B indicate that amplification products are obtained for this organism only with primer pair numbers 2157 and 2161.

A total of thirteen different genotypes of Staphylococcus aureus were identified according to the unique combinations of base compositions across the eight different bioagent identifying amplicons obtained with the eight primer pairs in Table 21A. These results indicate that this eight primer pair panel is useful for analysis of unknown or newly emerging strains of Staphylococcus aureus. It is expected that a kit comprising one or more of the members of the panels in Tables 21A and 21B will be a useful embodiment provided herein. It is envisioned that a kit comprising the primer pairs of Table 21C, or another combination of primer pairs from examples 14 and 15 would be a useful embodiment provided herein that could be useful in identification of Staphylococcus aureus bioagents at multiple levels.

### Example 16

### Selection and Use of Triangulation Genotyping Analysis Primer Pairs for Members of the Bacterial Genus Vibrio

To combine the power of high-throughput mass spectrometric analysis of bioagent identifying amplicons with the sub-species characteristic resolving power provided by triangulation genotyping analysis, a panel of eight triangulation genotyping analysis primer pairs was selected. The primer pairs are configured to produce bioagent identifying amplicons within seven different housekeeping genes which are listed in Table 23. The primer sequences are found in Table 2 and are cross-referenced by the primer pair numbers, primer pair names or SEQ ID NOs listed in Table 23.

TABLE 23
Primer Pairs for Triangulation Genotyping Analysis of Members of the Bacterial Genus Vibrio

| | | Forward | | Reverse | |
|---|---|---|---|---|---|
| Primer | | Primer | | Primer | |
| Pair | | (SEQ ID | | (SEQ ID | Target |
| No. | Forward Primer Name | NO:) | Reverse Primer Name | NO:) | Gene |
| 1098 | RNASEP_VBC_331_349_F | 325 | RNASEP_VBC_388_414_R | 1163 | RNAse P |
| 2000 | CTXB_NC002505_46_70_F | 278 | CTXB_NC002505_132_162_R | 1039 | ctxB |
| 2001 | FUR_NC002505_87_113_F | 465 | FUR_NC002505_205_228_R | 1037 | fur |
| 2011 | GYRB_NC002505_1161_1190_F | 148 | GYRB_NC002505_1255_1284_R | 1172 | gyrB |
| 2012 | OMPU_NC002505__85_110_F | 190 | OMPU_NC002505_154_180_R | 1254 | ompU |
| 2014 | OMPU_NC002505_431_455_F | 266 | OMPU_NC002505_544_567_R | 1094 | ompU |
| 2323 | CTXA_NC002505-1568114- | 508 | CTXA_NC002505-1568114- | 1297 | ctxA |
| | 1567341_122_149_F | | 1567341_186_214_R | | |
| 2927 | GAPA_NC002505_694_721_F | 259 | GAPA_NC_002505_29_58_R | 1060 | gapA |

A group of 50 bacterial isolates containing multiple strains of both environmental and clinical isolates of Vibrio cholerae, 9 other Vibrio species, and 3 species of Photobacteria were tested using this panel of primer pairs. Base compositions of amplification products obtained with these 8 primer pairs were used to distinguish amongst various species tested, including sub-species differentiation within Vibrio cholerae isolates. For instance, the non-O1/non-O139 isolates were clearly resolved from the O1 and the O139 isolates, as were several of the environmental isolates of Vibrio cholerae from the clinical isolates.

It is expected that a kit comprising one or more of the members of this panel will be a useful embodiment of the present invention.

### Example 17

### Selection and Use of Triangulation Genotyping Analysis Primer Pairs for Members of the Bacterial Genus Pseudomonas

To combine the power of high-throughput mass spectrometric analysis of bioagent identifying amplicons with the sub-species characteristic resolving power provided by triangulation genotyping analysis, a panel of twelve triangulation genotyping analysis primer pairs was selected. The primer pairs are configured to produce bioagent identifying amplicons within seven different housekeeping genes which are listed in Table 24. The primer sequences are found in Table 2 and are cross-referenced by the primer pair numbers, primer pair names or SEQ ID NOs listed in Table 24.

TABLE 24
Primer Pairs for Triangulation Genotyping Analysis of Members of the Bacterial Genus
Pseudomonas

| | | Forward | | Reverse | |
|---|---|---|---|---|---|
| Primer | | Primer | | Primer | |
| Pair | | (SEQ ID | | (SEQ ID | Target |
| No. | Forward Primer Name | NO:) | Reverse Primer Name | NO:) | Gene |
| 2949 | ACS_NC002516-970624- | 376 | ACS_NC002516-970624- | 1265 | acsA |
| | 971013_299_316_F | | 971013_364_383_R | | |
| 2950 | ARO_NC002516-26883- | 267 | ARO_NC002516-26883- | 1341 | aroE |
| | 27380_4_26_F | | 27380_111_128_R | | |
| 2951 | ARO_NC002516-26883- | 705 | ARO_NC002516-26883- | 1056 | aroE |
| | 27380_356_377_F | | 27380_459_484_R | | |
| 2954 | GUA_NC002516-4226546- | 710 | GUA_NC002516-4226546- | 1259 | guaA |
| | 4226174_155_178_F | | 4226174_265_287_R | | |
| 2956 | GUA_NC002516-4226546- | 374 | GUA_NC002516-4226546- | 1111 | guaA |
| | 4226174_242_263_F | | 4226174_355_371_R | | |
| 2957 | MUT_NC002516-5551158- | 545 | MUT_NC002516-5551158- | 978 | mutL |
| | 5550717_5_26_F | | 5550717_99_116_R | | |
| 2959 | NUO_NC002516-2984589- | 249 | NUO_NC002516-2984589- | 1095 | nuoD |
| | 2984954_8_26_F | | 2984954_97_117_R | | |
| 2960 | NUO_NC002516-2984589- | 195 | NUO_NC002516-2984589- | 1376 | nuoD |
| | 2984954_218_239_F | | 2984954_301_326_R | | |
| 2961 | PPS_NC002516-1915014- | 311 | PPS_NC002516-1915014- | 1014 | pps |
| | 1915383_44_63_F | | 1915383_140_165_R | | |
| 2962 | PPS_NC002516-1915014- | 365 | PPS_NC002516-1915014- | 1052 | pps |
| | 1915383_240_258_F | | 1915383_341_360_R | | |
| 2963 | TRP_NC002516-671831- | 527 | TRP_NC002516-671831- | 1071 | trpE |
| | 672273_24_42_F | | 672273_131_150_R | | |
| 2964 | TRP_NC002516-671831- | 490 | TRP_NC002516-671831- | 1182 | trpE |
| | 672273_261_282_F | | 672273_362_383_R | | |

It is expected that a kit comprising one or more of the members of this panel will be a useful embodiment of the present invention.

### Example 18

### Analysis Involving a Staphylococcus aureus tsst1 Gene Calibrant Polynucleotide

Primer pairs 3105, 3106, and 3107 were used in respective dilution series analyses in which the amplification target was a calibrant polynucleotide comprising a segment of the Staphylococcus aureus tsst1 gene. The individual primer sequences of primer pairs 3105, 3106, and 3107 are found in Table 2. Aside from varied calibrant polynucleotide copies numbers, the amplification reaction conditions, PCR product purification protocol, and base composition analysis utilized in this example were the same as those described in Examples 2-4, above. The results of this analysis are provided in Table 25, where the average calibrant polynucleotide copy numbers utilized in the various reactions are specified and “X” denotes that the calibrant polynucleotide was detected in the particular reaction mixture.

TABLE 25

| Primer | Calibrant Copy Number | | | | | | | | | | | |
|---|---|---|---|---|---|---|---|---|---|---|---|---|
| Pair No. | 0 | 4.8828125 | 9.765625 | 19.53125 | 39.0625 | 78.125 | 156.25 | 312.5 | 625 | 1250 | 2500 | 5000 |
| 3105 | | | X | X | X | X | X | X | X | X | X | X |
| 3106 | X | | X | X | X | X | X | X | X | X | X | X |
| 3107 | | | | | | | | | | X | X | X |

### Example 19

### Analysis of Isolated Clinical Samples

Primer pair 3106, which targets the Staphylococcus aureus tsst1 gene, was used against eight isolated clinical samples received from the CDC. The sequences of primer pair 3106 (i.e., SEQ ID NOS: 1465 and 1466) are found in Table 2. Each sample was analyzed in two parallel replicates, as was a control reaction that only included a calibrant polynucleotide (i.e., the control was the same as the other replicates aside from lacking DNA from any of the clinical samples). The amplification reaction conditions, PCR product purification protocol, and base composition analysis utilized in this example were the same as those described in Examples 2-4, above. The results of this analysis are provided in Table 26. As expected, two of the clinical samples were positive for Staphylococcus aureus (i.e., CDC0011 and CDC0021).

TABLE 26
Primer Pair 3106

| Sample | Well 1 | Well 2 | Result |
|---|---|---|---|
| calibrant only | failed | negative | negative |
| CDC004 | failed | failed | failed |
| CDC007 | failed | negative | negative |
| CDC0011 | positive | positive | positive |
| CDC0012 | negative | negative | negative |
| CDC0014 | negative | failed | negative |
| CDC0015 | negative | negative | negative |
| CDC0021 | positive | positive | positive |
| CDC0027 | failed | negative | negative |

The present invention includes any combination of the various species and subgeneric groupings falling within the generic disclosure. This invention therefore includes the generic description of the invention with a proviso or negative limitation removing any subject matter from the genus, regardless of whether or not the excised material is specifically recited herein.

While in accordance with the patent statutes, description of the various embodiments and examples have been provided, the scope of the invention is not to be limited thereto or thereby. Modifications and alterations of the present invention will be apparent to those skilled in the art without departing from the scope and spirit of the present invention.

Therefore, it will be appreciated that the scope of this invention is to be defined by the appended claims, rather than by the specific examples which have been presented by way of example.

Each reference (including, but not limited to, journal articles, U.S. and non-U.S. patents, patent application publications, international patent application publications, gene bank gi or accession numbers, internet web sites, and the like) cited in the present application is incorporated herein by reference in its entirety.

What is claimed is:

1. A kit comprising an oligonucleotide primer pair comprising a forward primer and a reverse primer, each comprising between 13 and 35 linked nucleotides in length, wherein:
the forward primer comprises at least 70% sequence identity with SEQ ID NO.: 1465 and the reverse primer comprises at least 70% sequence identity with SEQ ID NO.: 1466,
the forward primer comprises at least 70% sequence identity with SEQ ID NO.: 1467 and the reverse primer comprises at least 70% sequence identity with SEQ ID NO.: 1468, or
the forward primer comprises at least 70% sequence identity with SEQ ID NO.: 1469 and the reverse primer comprises at least 70% sequence identity with SEQ ID NO.: 1470.

2. The kit of claim 1, further comprising at least one additional oligonucleotide primer pair that is configured to generate an amplicon between 45 and 200 linked nucleotides in length, and comprises a forward and a reverse primer, each comprising between 13 and 35 linked nucleotides in length and each configured to hybridize to conserved sequence regions within a Staphylococcus aureus gene, said gene selected from the group consisting of: ermA, ermC, pvluk, nuc, tufB, mecA, mec-R1, tsst1, and mupR.

3. The kit of claim 2, wherein said oligonucleotide primer pair and said at least one additional oligonucleotide primer pair comprises eight primer pairs, said eight oligonucleotide primer pairs having at least 70% sequence identity to: SEQ ID NO.: 288:SEQ ID NO.:1269, SEQ ID NO.: 698:SEQ ID NO.:1420, SEQ ID NO.: 217:SEQ ID NO.:1167, SEQ ID NO.: 399:SEQ ID NO.:1041, SEQ ID NO.: 456:SEQ ID NO.:1261, SEQ ID NO.: 430:SEQ ID NO.:1321, SEQ ID NO.: 174:SEQ ID NO.:853, and SEQ ID NO.: 1465:SEQ ID NO.:1466, SEQ ID NO.: 1467:SEQ ID NO.:1468, or SEQ ID NO.: 1469:SEQ ID NO.:1470.

4. The kit of claim 3 wherein said eight oligonucleotide primers consist of SEQ ID NO.: 288:SEQ ID NO.:1269, SEQ ID NO.: 698:SEQ ID NO.:1420, SEQ ID NO.: 217:SEQ ID NO.:1167, SEQ ID NO.: 399:SEQ ID NO.:1041, SEQ ID NO.: 456:SEQ ID NO.:1261, SEQ ID NO.: 430:SEQ ID NO.:1321, SEQ ID NO.: 174:SEQ ID NO.:853, and SEQ ID NO.: 1465:SEQ ID NO.:1466, SEQ ID NO.: 1467:SEQ ID NO.:1468, or SEQ ID NO.: 1469:SEQ ID NO.:1470.

5. The kit of claim 4 further comprising eight additional primer pairs, said eight additional primer pairs comprising at least 70% sequence identity with: SEQ ID NO.: 437:SEQ ID NO.:1232, SEQ ID NO.: 530:SEQ ID NO.:891, SEQ ID NO.: 474:SEQ ID NO.:869, SEQ ID NO.: 268:SEQ ID NO.:1284, SEQ ID NO.: 418:SEQ ID NO.:1301, SEQ ID NO.: 318:SEQ ID NO.:1300, SEQ ID NO.: 440:SEQ ID NO.:1076, and SEQ ID NO.: 219:SEQ ID NO.:1013.

6. An oligonucleotide primer pair comprising a forward primer and a reverse primer, each comprising between 13 and 35 linked nucleotides in length, wherein the forward primer comprises at least 70% sequence identity with SEQ ID NO.: 1465, SEQ ID NO.: 1467, or SEQ ID NO.: 1469.

7. The oligonucleotide primer pair of claim 6, wherein said forward primer comprises at least 80% sequence identity with SEQ ID NO.: 1465, SEQ ID NO.: 1467, or SEQ ID NO.: 1469.

8. The oligonucleotide primer pair of claim 6, wherein said forward primer comprises at least 90% sequence identity with SEQ ID NO.: 1465, SEQ ID NO.: 1467, or SEQ ID NO.: 1469.

9. The oligonucleotide primer pair of claim 6, wherein said forward primer comprises at least 95% sequence identity with SEQ ID NO.: 1465, SEQ ID NO.: 1467, or SEQ ID NO.: 1469.

10. The oligonucleotide primer pair of claim 6, wherein said forward primer comprises at least 100% sequence identity with SEQ ID NO.: 1465, SEQ ID NO.: 1467, or SEQ ID NO.: 1469.

11. The oligonucleotide primer pair of claim 6, wherein said forward primer is SEQ ID NO.: 1465, SEQ ID NO.: 1467, or SEQ ID NO.: 1469 with 0-10 nucleobase deletions, insertions and/or substitutions.

12. The oligonucleotide primer pair of claim 6, wherein said forward primer is SEQ ID NO.: 1465, SEQ ID NO.: 1467, or SEQ ID NO.: 1469.

13. A composition comprising the oligonucleotide primer of claim 6.

14. The oligonucleotide primer pair of claim 6, wherein at least one of said forward primer and said reverse primer comprises at least one modified nucleobase.

15. The oligonucleotide primer pair of claim 14, wherein at least one of said at least one modified nucleobase is a mass modified nucleobase.

16. The oligonucleotide primer pair of claim 15, wherein said mass modified nucleobase is 5-Iodo-C.

17. The oligonucleotide primer pair of claim 15, wherein said mass modified nucleobase comprises a molecular mass modifying tag.

18. The oligonucleotide primer pair of claim 14, wherein at least one of said at least one modified nucleobase is a universal nucleobase.

19. The oligonucleotide primer pair of claim 18, wherein said universal nucleobase is inosine.

20. The oligonucleotide primer pair of claim 6, wherein at least one of said forward primer and said reverse primer comprises a non-templated T residue at its 5′ end.

21. An oligonucleotide primer pair comprising a forward primer and a reverse primer, each comprising between 13 and 35 linked nucleotides in length, wherein the reverse primer comprises at least 70% sequence identity with SEQ ID NO.: 1466, SEQ ID NO.: 1468, or SEQ ID NO.: 1470.

22. The oligonucleotide primer pair of claim 13, wherein said reverse primer comprises at least 80% sequence identity with SEQ ID NO.: 1466, SEQ ID NO.: 1468, or SEQ ID NO.: 1470.

23. The oligonucleotide primer pair of claim 13, wherein said reverse primer comprises at least 90% sequence identity with SEQ ID NO.: 1466, SEQ ID NO.: 1468, or SEQ ID NO.: 1470.

24. The oligonucleotide primer pair of claim 13, wherein said reverse primer comprises at least 95% sequence identity with SEQ ID NO.: 1466, SEQ ID NO.: 1468, or SEQ ID NO.: 1470.

25. The oligonucleotide primer pair of claim 13, wherein said reverse primer comprises at least 100% sequence identity with SEQ ID NO.: 1466, SEQ ID NO.: 1468, or SEQ ID NO.: 1470.

26. The oligonucleotide primer pair of claim 13, wherein said reverse primer is SEQ ID NO.: 1466, SEQ ID NO.: 1468, or SEQ ID NO.: 1470 with 0-10 nucleobase deletions, insertions and/or substitutions.

27. The oligonucleotide primer pair of claim 13, wherein said reverse primer is SEQ ID NO.: 1466, SEQ ID NO.: 1468, or SEQ ID NO.: 1470.

28. A composition comprising the oligonucleotide primer of claim 21.

29. The oligonucleotide primer pair of claim 21, wherein at least one of said forward primer and said reverse primer comprises at least one modified nucleobase.

30. The oligonucleotide primer pair of claim 29, wherein at least one of said at least one modified nucleobase is a mass modified nucleobase.

31. The oligonucleotide primer pair of claim 30, wherein said mass modified nucleobase is 5-Iodo-C.

32. The oligonucleotide primer pair of claim 30, wherein said mass modified nucleobase comprises a molecular mass modifying tag.

33. The oligonucleotide primer pair of claim 29, wherein at least one of said at least one modified nucleobase is a universal nucleobase.

34. The oligonucleotide primer pair of claim 33, wherein said universal nucleobase is inosine.

35. The oligonucleotide primer pair of claim 21, wherein at least one of said forward primer and said reverse primer comprises a non-templated T residue at its 5′ end.

36. A method for identifying a Staphylococcus aureus bioagent in a sample comprising:
a) amplifying a nucleic acid from said sample using an oligonucleotide primer pair comprising a forward primer and a reverse primer, each comprising between 13 and 35 linked nucleotides in length, said forward primer comprising at least 70% sequence identity with SEQ ID NO.: 1465, SEQ ID NO.: 1467, or SEQ ID NO.: 1469 and said reverse primer comprising at least 70% sequence identity with SEQ ID NO.: 1466, SEQ ID NO.: 1468, or SEQ ID NO.: 1470, wherein said amplifying generates at least one amplification product that comprises between 45 and 200 linked nucleotides; and
b) determining the molecular mass of said at least one amplification product by mass spectrometry.

37. The method of claim 36, further comprising comparing said determined molecular mass to a database comprising a plurality of molecular masses of bioagent identifying amplicons, wherein a match between said determined molecular mass and a molecular mass comprised in said database identifies said Staphylococcus aureus bioagent in said sample.

38. The method of claim 36, further comprising calculating a base composition of said at least one amplification product using said molecular mass.

39. The method of claim 38, further comprising comparing said calculated base composition to a database comprising a plurality of base compositions of bioagent identifying amplicons, wherein a match between said calculated base composition and a base composition comprised in said database identifies said Staphylococcus aureus bioagent in said sample.

40. The method of claim 36, further comprising repeating said amplifying and determining steps using at least one additional oligonucleotide primer pair wherein the primers of each of said at least one additional primer pair are configured to hybridize to conserved sequence regions within a Staphylococcus aureus gene selected from the group consisting ermA, ermC, pvluk, nuc, tufB, mecA, mec-R1, tsst1, and mupR.

41. The method of claim 36, wherein said identifying comprises detecting the presence of said Staphylococcus aureus bioagent in said sample.

42. The method of claim 36, wherein said identifying comprises determining the presence or absence of virulence of said Staphylococcus aureus bioagent in said sample.
